(19) United States
(12) Patent Application Publication
(10) Pub. No.: US 2025/0122306 A1
(43) Pub. Date: Apr. 17, 2025

(54) ANTI-TISSUE FACTOR ANTIBODIES AND ANTIBODY CONJUGATES, COMPOSITIONS COMPRISING ANTI-TISSUE FACTOR ANTIBODIES OR ANTIBODY CONJUGATES, AND METHODS OF MAKING AND USING ANTI-TISSUE FACTOR ANTIBODIES AND ANTIBODY CONJUGATES

(71) Applicant: SUTRO BIOPHARMA, INC., South San Francisco, CA (US)

(72) Inventors: Grace Jungeun LEE, Pacifica, CA (US); Amandeep Kaur GAKHAL, San Mateo, CA (US); Daniel CALARESE, Millbrae, CA (US); Junhao YANG, Palo Alto, CA (US); Krishna BAJJURI, Union City, CA (US); Xiaofan LI, Belmont, CA (US); Sihong ZHOU, San Mateo, CA (US); Helena KIEFEL, South San Francisco, CA (US); Robert Tian-Xuan YUAN, San Mateo, CA (US); Andrew James MCGEEHAN, Mill Valley, CA (US); Miao WEN, Union City, CA (US); Gang YIN, South San Francisco, CA (US); Alice yAM, Belmont, CA (US)

(21) Appl. No.: 18/914,023

(22) Filed: Oct. 11, 2024

Related U.S. Application Data
(60) Provisional application No. 63/590,343, filed on Oct. 13, 2023.
(60) Provisional application No. 63/684,699, filed on Aug. 19, 2024.

(51) Int. Cl. C07K 16/36; A61K 47/68; A61P 35/00

(52) U.S. Cl. CPC C07K 16/36; A61K 47/6803; A61K 47/6851; A61P 35/00; C07K 2317/24; C07K 2317/732; C07K 2317/92

(57) ABSTRACT

The present disclosure relates to antibodies and antibody conjugates that selectively bind to tissue factor and its isoforms and homologs, and compositions comprising the antibodies. Also provided are methods of using the antibodies and antibody conjugates, such as therapeutic and diagnostic methods.

### CROSS-REFERENCE TO RELATED APPLICATIONS

This application claims the benefit of U.S. Provisional Application No. 63/590,343, filed Oct. 13, 2023, and U.S. Provisional Application No. 63/684,699, filed Aug. 19, 2024. Each of these applications are incorporated for all purposes in their entirety.

### REFERENCE TO ELECTRONIC SEQUENCE LISTING

The application contains a Sequence Listing which has been submitted electronically in .XML format and is hereby incorporated by reference in its entirety. Said. XML copy, created on Oct. 7, 2024, is named “108843.00506.xml” and is 1,460,613 bytes in size. The sequence listing contained in this .XML file is part of the specification and is hereby incorporated by reference herein in its entirety.

### FIELD OF THE INVENTION

The present disclosure generally relates to antibodies and antibody conjugates with binding specificity for tissue factor and compositions comprising the antibodies or antibody conjugates, including pharmaceutical compositions, diagnostic compositions, and kits. Also provided are methods of making anti-tissue factor antibodies and antibody conjugates, and methods of using anti-tissue factor antibodies and antibody conjugates, for example, for therapeutic purposes, diagnostic purposes, and research purposes.

### BACKGROUND

Tissue factor (TF), also known as CD142, is a cellular membrane glycoprotein. Tissue factor is sometimes referred to as Coagulation Factor III because it is the primary initiator of blood coagulation, where complexation of Factor VIIa and tissue factor (TF:FVIIa complex) activates the coagulation protease cascade. Mackman, et al., 2004, Atherosclerosis, Thrombosis, and Vascular Biology 24:1015-1022. TF is expressed in the subendothelium. When endothelium is damaged, tissue factor combines with circulating factor VII to activate factor X. Activated factor X initiates the coagulation cascade, allowing clot formation.

Aberrant expression of TF plays a role in thrombosis in disease, including sepsis, atherosclerosis, and cancer. TF has also been implicated in inflammation, angiogenesis, metastasis, and cell migration. Mackman, et al., 2004, Atherosclerosis, Thrombosis, and Vascular Biology 24:1015-1022. TF is broadly expressed across multiple solid tumor indications.

Accordingly, there is a need for improved methods of modulating the activity of TF and the downstream signaling coagulation processes activated by TF. Moreover, given the implication of TF in thrombosis, there is a need for improved therapeutics that specifically target cells and tissues that express TF. Antibody conjugates to TF could be used to deliver therapeutic or diagnostic payload moieties to target cells expressing TF for the treatment or diagnosis of such diseases.

### SUMMARY

Provided herein are antibodies that selectively bind tissue factor (TF). In some embodiments, the antibodies bind human TF. In some embodiments, the antibodies also bind homologs of human TF. In some aspects, the homologs include a cynomolgus monkey homolog.

Also provided herein are antibody conjugates that selectively bind TF. The antibody conjugates comprise an antibody that binds TF linked to one or more payload moieties. The antibody can be linked to the payload directly by a covalent bond or indirectly by way of a linker. TF antibodies are described in detail herein, as are useful payload moieties, and useful linkers.

In some embodiments, the antibodies or antibody conjugates comprise an illustrative CDR, V_{H}, V_{L}, HC, or LC sequence provided in this disclosure, or a variant thereof. In some aspects, the variant is a variant with one or more conservative amino acid substitutions. In some aspects, the variant has sequence identity to the illustrative sequence or sequences.

In another aspect, provided are compositions comprising the antibodies or antibody conjugates. In some embodiments, the compositions are pharmaceutical compositions. In some embodiments, the pharmaceutical composition is for parenteral administration. Any suitable pharmaceutical composition may be used. In some embodiments, the pharmaceutical composition is a composition for parenteral administration. In a further aspect, provided herein are kits comprising the antibodies or antibody conjugates or pharmaceutical compositions.

This disclosure also provides methods of using the anti-TF antibodies or antibody conjugates provided herein. In some embodiments, the methods are methods of delivering one or more payload moieties to a target cell or tissue expressing TF. In some embodiments, the method is a method of treatment. In some embodiments, the method is a diagnostic method. In some embodiments, the method is an analytical method. In some embodiments, the method is a method of purifying and/or quantifying TF.

In some embodiments, the antibodies, antibody conjugates, or pharmaceutical compositions thereof are used to treat a disease or condition. In some aspects, the disease or condition is a TF-expressing malignancy. In some aspects, the disease or condition is selected from a cancer, autoimmune disease, and infection.

In some embodiments, the antibodies or antibody conjugates bind human TF. In some embodiments, the antibodies or antibody conjugates also bind homologs of human TF. In some aspects, the antibodies or antibody conjugates also bind homologs of cynomolgus monkey and/or mouse TF.

These and other embodiments of the invention along with many of its features are described in more detail in conjunction with the text below and attached figures.

### BRIEF DESCRIPTION OF THE FIGURES

FIG. 1A provides a comparison of the Kabat and Chothia numbering systems for CDR-H1. Adapted from Martin A. C. R. (2010). Protein Sequence and Structure Analysis of Antibody Variable Domains. In R. Kontermann & S. Dübel (Eds.), Antibody Engineering vol. 2 (pp. 33-51). Springer-Verlag, Berlin Heidelberg.

FIG. 1B provides a general schematic design for a representative antibody conjugate of the present disclosure where 1 is TF, 2 is a non-natural amino acid (pAMF), 3 is a β-glucuronidase linker and 4 is an exatecan payload.

FIG. 2 provides thrombin production data for Conjugate 1 and Conjugate 26.

FIG. 3A provides HPLC-SEC results for formulation buffer comparison for Conjugate 7 at 4° C., 25° C. and 37° C. conditions.

FIG. 3B provides HPLC-SEC results for Conjugate 2 at 4° C., 25° C. and 37° C. conditions.

FIG. 4 provides HPLC-SEC results over 5× freeze/thaw cycles for Conjugate 2 in one formulation buffer and for Conjugate 7 in three different formulation buffers.

FIG. 5 provides the viability of CD66b+ cells as a percentage relative to PBS control for Neutrophil precursor cells treated with Conjugates 2, 3 and 6.

FIG. 6A provides measured DAR of Conjugates 2, 6 and 7 over 21 days.

FIG. 6B provides deconvoluted mass spectra of anti-TF ADC samples from in vivo linker payload stability study for Conjugates 2, 6 and 7.

FIGS. 7A-C provide HCC1954 tumor growth curves in response to treatment with a single i.v. dose of TF-targeted ADCs, with doses (FIG. 7A) 0.5 mg/kg or (FIG. 7B) 5 mg/kg. FIG. 7C provides a scatter plot of individual tumor volumes on day 35 post treatment, when control tumors reached the study endpoint. Arrows represent dosing days. Statistical analysis was performed on tumor volumes on day 35 using one-way ANOVA with Dunnett's multiple comparisons test versus the vehicle group. A probability of less than 5% (p<0.05) was considered significant. ****=p<0.0001. All graphs are presented as individual values or mean±SEM.

FIGS. 8A-B provide (FIG. 8A) H1975 tumor growth curves in response to treatment with a single i.v. dose of TF-targeted ADCs at 1 mg/kg or 2 mg/kg. (FIG. 8B) Scatter plot of individual tumor volumes on day 17 post treatment, when control tumors reached the study endpoint. Arrows represent dosing days. Statistical analysis was performed on tumor volumes on day 17 using one-way ANOVA with Dunnett's multiple comparisons test versus the vehicle group. A probability of less than 5% (p<0.05) was considered significant. *=p<0.0001. All graphs are presented as individual values or mean±SEM.

FIGS. 9A-B provide (FIG. 9A) H1975 tumor growth curves in response to treatment with a single i.v. dose of TF-targeted ADCs at doses ranging from 0.5 mg/kg to 2 mg/kg. (FIG. 9B) Scatter plot of individual tumor volumes on day 18 post treatment, when control tumors reached the study endpoint. Arrows represent dosing days. Statistical analysis was performed on tumor volumes on day 18 using one-way ANOVA with Dunnett's multiple comparisons test versus the vehicle group. A probability of less than 5% (p<0.05) was considered significant. ****=p<0.0001. All graphs are presented as individual values or mean±SEM.

FIGS. 10A-D provide (FIG. 10A) MDA-MB-231 tumor growth curves in response to treatment with two weekly i.v. doses (qwx2) of TF-targeted ADCs at doses ranging from 0.5 mg/kg to 2 mg/kg. (FIG. 10B) Scatter plot of individual tumor volumes on day 42 post treatment, when control tumors reached the study endpoint. Arrows represent dosing days. Statistical analysis was performed on tumor volumes on day 42 using one-way ANOVA with Dunnett's multiple comparisons test versus the vehicle group. A probability of less than 5% (p<0.05) was considered significant. ****=p<0.0001. All graphs are presented as individual values or mean±SEM. (FIG. 10C) MDA-MB-231 tumor growth curves in response to treatment with two weekly i.v. doses (qwx2) of a TF-targeted ADC at doses ranging from 0.25 mg/kg to 1 mg/kg. (FIG. 10D) Scatter plot of individual tumor volumes on day 41 post treatment, when control tumors reached the study endpoint. Arrows represent dosing days. Statistical analysis was performed on tumor volumes on day 41 using one-way ANOVA with Dunnett's multiple comparisons test versus the vehicle group. A probability of less than 5% (p<0.05) was considered significant. ****=p<0.0001. All graphs are presented as individual values or mean±SEM.

FIGS. 11A-D provide (FIG. 11A) H1975 tumor growth curves in response to treatment with a single i.v. dose of TF-targeted ADCs at doses ranging from 0.25 mg/kg to 2 mg/kg. (FIG. 11B) Scatter plot of individual tumor volumes on day 24 post treatment, when control tumors reached the study endpoint. Arrows represent dosing days. Statistical analysis was performed on tumor volumes on day 24 using one-way ANOVA with Dunnett's multiple comparisons test versus the vehicle group. A probability of less than 5% (p<0.05) was considered significant. ***=p<0.001; ****=p<0.0001. All graphs are presented as individual values or mean±SEM. (FIG. 11C) H1975 tumor growth curves in response to treatment with a single i.v. dose of TF-targeted ADCs at doses ranging from 0.125 mg/kg to 1 mg/kg. (FIG. 11D) Scatter plot of individual tumor volumes on day 25 post treatment, when control tumors reached the study endpoint. Statistical analysis was performed on tumor volumes on day 25 using one-way ANOVA with Dunnett's multiple comparisons test versus the vehicle group. A probability of less than 5% (p<0.05) was considered significant. **=p<0.01; ****=p<0.0001. All graphs are presented as individual values or mean±SEM.

FIGS. 12A-D provide (FIG. 12A) Detroit562 tumor growth curves in response to treatment with a single i.v. dose of Conjugate 7 at 1 mg/kg or 2 mg/kg. (FIG. 12B) Scatter plot of individual tumor volumes on day 21 post treatment, when control tumors reached the study endpoint. Arrow represents dosing day. Statistical analysis was performed on tumor volumes on day 21 using one-way ANOVA with Dunnett's multiple comparisons test versus the vehicle group. A probability of less than 5% (p<0.05) was considered significant. ***=p<0.0001. All graphs are presented as individual values or mean±SEM. (FIG. 12C) Detroit562 tumor growth curves in response to treatment with a single i.v. dose of TF-targeted ADCs at doses ranging from 0.25 mg/kg to 2 mg/kg. (FIG. 12D) Scatter plot of individual tumor volumes on day 21 post treatment, when control tumors reached the study endpoint. Statistical analysis was performed on tumor volumes on day 21 using one-way ANOVA with Dunnett's multiple comparisons test versus the vehicle group. A probability of less than 5% (p<0.05) was considered significant. ****=p<0.0001. All graphs are presented as individual values or mean±SEM.

FIGS. 13A-B provide (FIG. 13A) HCT-116 tumor growth curves in response to treatment with a single i.v. dose of TF-targeted ADCs at doses ranging from 2.5 mg/kg to 15 mg/kg. (FIG. 13B) Scatter plot of individual tumor volumes on day 21 post treatment, when control tumors reached the study endpoint. Statistical analysis was performed on tumor volumes on day 21 using one-way ANOVA with Dunnett's multiple comparisons test versus the vehicle group. A probability of less than 5% (p<0.05) was considered significant. ****=p<0.0001. All graphs are presented as individual values or mean±SEM.

FIGS. 14A-D provide (FIG. 14A) MC38-hTF tumor growth curves in response to treatment with a single i.v. dose of Conjugate 7 at 10 mg/kg, multiple i.p. doses of anti-PD-1 at 10 mg/kg, or combination treatment. Number of complete responses (CRs) are designated to the right of the tumor growth curves. (FIG. 14B) Scatter plot of individual tumor volumes on day 6 post treatment, when control tumors reached the study endpoint. Arrows represent dosing days. Statistical analysis was performed on tumor volumes on day 6 using one-way ANOVA with Dunnett's multiple comparisons test versus the vehicle group. A probability of less than 5% (p<0.05) was considered significant. **=p<0.01; ****=p<0.0001. All graphs are presented as individual values or mean±SEM. (FIG. 14C) MC38-hTF tumor growth curves in response to treatment with a single i.v. dose of Conjugate 35 at 5 mg/kg, multiple i.p. doses of anti-PD-1 at 10 mg/kg, or combination treatment. Number of complete responses (CRs) are designated to the right of the tumor growth curves. (FIG. 14D) Scatter plot of individual tumor volumes on day 8 post treatment, when control tumors reached the study endpoint. Arrows represent dosing days. Statistical analysis was performed on tumor volumes on day 8 using one-way ANOVA with Dunnett's multiple comparisons test versus the vehicle group. A probability of less than 5% (p<0.05) was considered significant. ****=p<0.0001. All graphs are presented as individual values or mean±SEM.

FIGS. 15A-B provide (FIG. 15A) Tumor growth curves of a NSCLC PDX model in response to treatment with a single i.v. dose of Conjugate 7 at doses ranging from 0.3 mg/kg to 10 mg/kg. (FIG. 15B) Scatter plot of individual tumor volumes on day 37 post treatment, when control tumors reached the study endpoint. Arrow represents dosing day. Statistical analysis was performed on tumor volumes on day 37 using one-way ANOVA with Dunnett's multiple comparisons test versus the vehicle group. A probability of less than 5% (p<0.05) was considered significant. ****=p<0.01; ****=p<0.001. All graphs are presented as individual values or mean±SEM.

### DETAILED DESCRIPTION OF THE EMBODIMENTS

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or ±1%. In certain embodiments, the term “about” indicates the designated value±one standard deviation of that value.

The term “combinations thereof” includes every possible combination of elements to which the term refers to. For example, a sentence stating “A, B, C, and combinations thereof” includes the following combinations: (1) A, (2) A and B, (3) A, B and C, (4) A and C, (5) B, (6) B and C, and (7) C.

The terms “tissue factor,” “TF,” and “TF antigen” are used interchangeably herein. Unless specified otherwise, the terms include any variants, isoforms and species homologs of human tissue factor that are naturally expressed by cells, or that are expressed by cells transfected with a tissue factor gene. Tissue factor proteins include, for example, human tissue factor (SEQ ID NO: 1). In some embodiments, tissue factor proteins include cynomolgus monkey tissue factor (SEQ ID NO: 2). In some embodiments, tissue factor proteins include murine tissue factor (SEQ ID NO: 3160). In some embodiments, tissue factor proteins include human tissue factor isoform 2 precursor (SEQ ID NO: 3161). In some embodiments, tissue factor proteins include murine tissue factor precursor (SEQ ID NO: 3162).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer. A “tissue factor antibody,” “anti-tissue factor antibody,” “tissue factor Ab,” “tissue factor-specific antibody” or “anti-tissue factor Ab” or the equivalent is an antibody, as described herein, which binds specifically to the antigen tissue factor. In some embodiments, the antibody binds the extracellular domain of tissue factor.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

TABLE 1
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

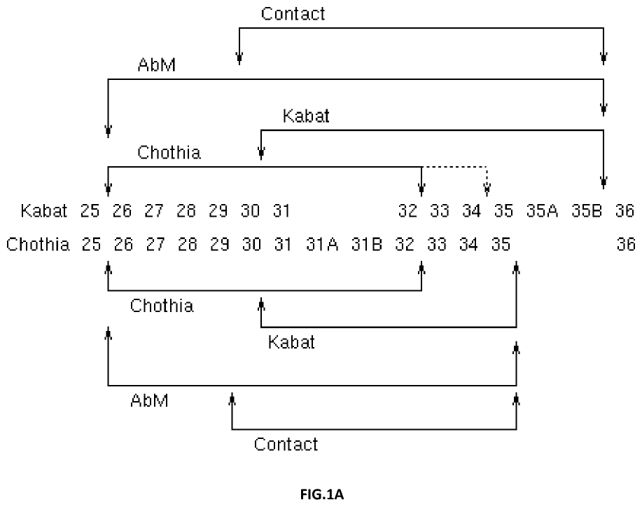

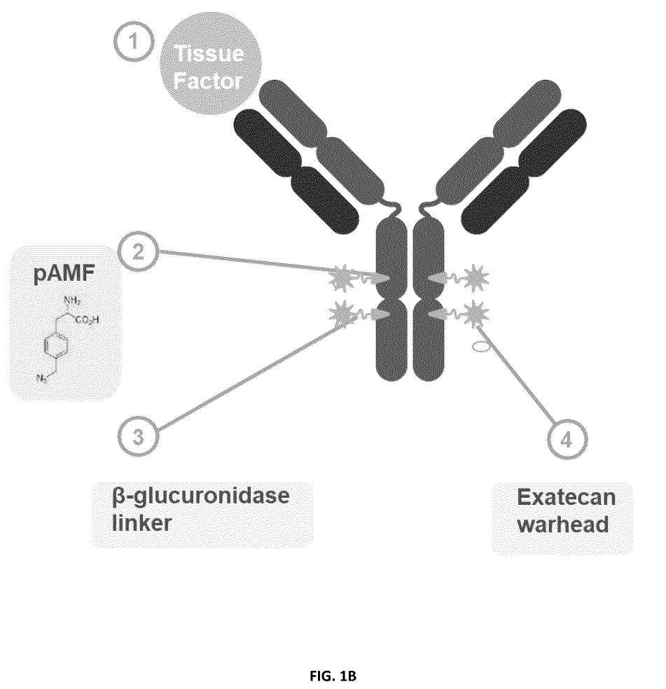

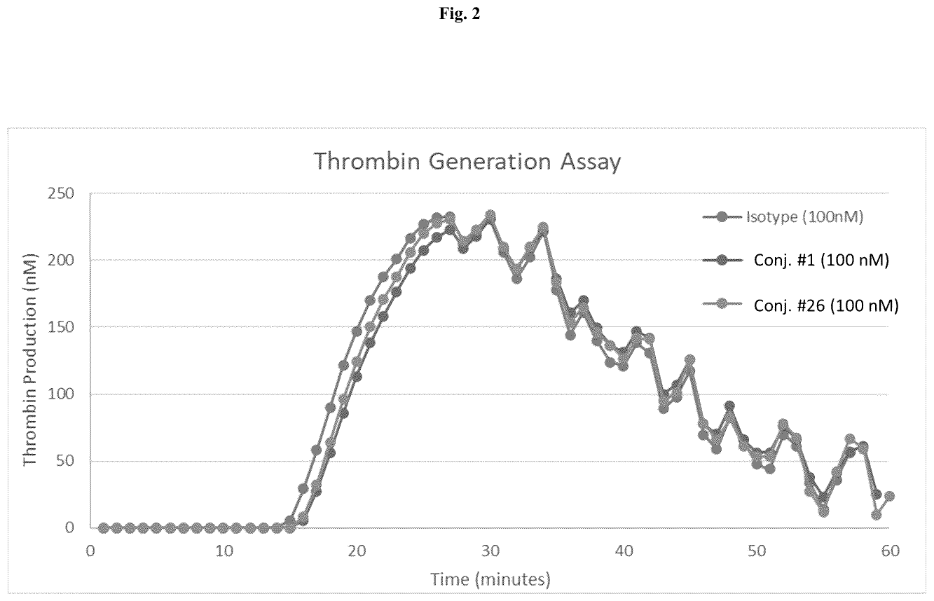

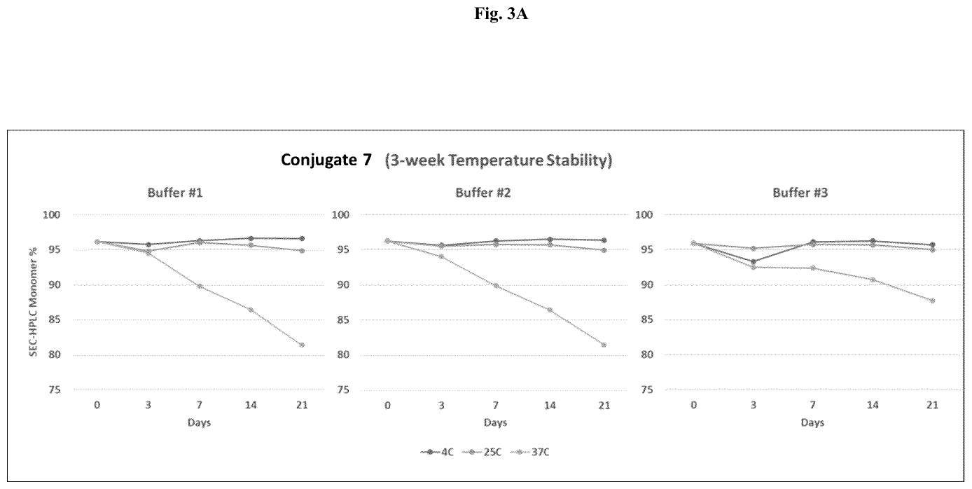

*The C-terminus of CDR-H1, when numbered using the Kabat numbering convention, varies between H32 and H34, depending on the length of the CDR, as illustrated in FIG. 1A.

Unless otherwise specified, the numbering scheme used for identification of a particular CDR herein is the Kabat/Chothia numbering scheme. Where the residues encompassed by these two numbering schemes diverge (e.g., CDR-H1 and/or CDR-H2), the numbering scheme is specified as either Kabat or Chothia. For convenience, CDR-H3 is sometimes referred to herein as either Kabat or Chothia. However, this is not intended to imply differences in sequence where they do not exist, and one of skill in the art can readily confirm whether the sequences are the same or different by examining the sequences.

CDRs may be assigned, for example, using antibody numbering software, such as Abnum, available at http://www.bioinf.org.uk/abs/abnum/, and described in Abhinandan and Martin, Immunology, 2008, 45:3832-3839, incorporated by reference in its entirety.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with ß-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). In some embodiments, the linker is SEQ ID NO: 3071. Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminus of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG1 Fc domain. In some embodiments, the IgG1 Fc domain comprises SEQ ID NO: 3062, or a portion thereof, or SEQ ID NO: 3068. SEQ ID NO: 3062 provides the sequence of C_{H1}, C_{H2}, and C_{H3} of the human IgG1 constant region. In some embodiments, the constant region can comprise sequences selected from SEQ ID NOs: 3062, 3063, 3064, and 3065. SEQ ID NO: 3068 provides the sequence of the constant region used in the illustrative scFv-Fc antibodies provided herein. In some embodiments, the sequence of the constant region can be linked to the scFv by a linker such as, by way of example, but not limitation, the linker of SEQ ID NO: 3071. In some embodiments, the linker can be N-terminal to the sequence of SEQ ID NO: 3068. In some embodiments, the constant region can further include a FlagHis Tag, such as that of SEQ ID NO: 3069 which can, by way of example but not limitation, be C-terminal to the Fc sequence.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody is prepared by at least one purification step.

In some embodiments, an isolated antibody is purified to at least 80%, 85%, 90%, 95%, or 99% by weight. In some embodiments, an isolated antibody is purified to at least 80%, 85%, 90%, 95%, or 99% by volume. In some embodiments, an isolated antibody is provided as a solution comprising at least 85%, 90%, 95%, 98%, 99% to 100% by weight. In some embodiments, an isolated antibody is provided as a solution comprising at least 85%, 90%, 95%, 98%, 99% to 100% by volume.

The term “non-natural amino acid” refers to an amino acid that is not a proteinogenic amino acid, or a post-translationally modified variant thereof. In particular, the term refers to an amino acid that is not one of the 20 common amino acids or pyrrolysine or selenocysteine, or post-translationally modified variants thereof.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can be represented by the dissociation constant (K_{D}). Affinity can be measured by common methods known in the art, including those described herein. Affinity can be determined, for example, using surface plasmon resonance (SPR) technology, such as a Biacore® instrument. In some embodiments, the affinity is determined at 25° C.

With regard to the binding of an antibody to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that mimics the antibody binding site on the target. In that case, specific binding is indicated if the binding of the antibody to the target is competitively inhibited by the control molecule.

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ.

The term “K_{A}” (M-1), as used herein, refers to the association equilibrium constant of a particular antibody-antigen interaction. K_{A}=kₐ/k_{d}.

An “affinity matured” antibody is one with one or more alterations in one or more CDRs or FRs that result in an improvement in the affinity of the antibody for its antigen, compared to a parent antibody which does not possess the alteration(s). In one embodiment, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

When used herein in the context of two or more antibodies, the term “competes with” or “cross-competes with” indicates that the two or more antibodies compete for binding to an antigen (e.g., TF). In one exemplary assay, TF is coated on a plate and allowed to bind a first antibody, after which a second, labeled antibody is added. If the presence of the first antibody reduces binding of the second antibody, then the antibodies compete. In another exemplary assay, a first antibody is coated on a plate and allowed to bind the antigen, and then the second antibody is added. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 50%, at least 60%, at least 70%, at least 80%, or at least 90%.

The term “epitope” means a portion of an antigen capable of specific binding to an antibody. Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter is lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to TF variants with different point-mutations, or to chimeric TF variants as described further in the Examples provided herein.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of an amino acid with a chemically or functionally similar amino acid. Conservative substitution tables providing similar amino acids are well known in the art. Polypeptide sequences having such substitutions are known as “conservatively modified variants.” Such conservatively modified variants are in addition to and do not exclude polymorphic variants, interspecies homologs, and alleles. By way of example, the groups of amino acids provided in Tables 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |
| Alcohol Group-Containing Residues | S and T |
| Aliphatic Residues | I, L, V, and M |
| Cycloalkenyl-associated Residues | F, H, W, and Y |
| Hydrophobic Residues | A, C, F, G, H, I, L, M, R, T, V, |
| | W, and Y |
| Negatively Charged Residues | D and E |
| Polar Residues | C, D, E, H, K, N, Q, R, S, and T |
| Positively Charged Residues | H, K, and R |
| Small Residues | A, C, D, G, N, P, S, T, and V |
| Very Small Residues | A, G, and S |
| Residues Involved in Turn Formation | A, C, D, E, G, H, K, N, Q, R, S, |
| | P, and T |
| Flexible Residues | Q, T, K, S, G, P, D, E, and R |

TABLE 3
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W.H. Freeman & Co., New York, NY. An antibody generated by making one or more conservative substitutions of amino acid residues in a parent antibody is referred to as a “conservatively modified variant.”

The term “payload” refers to a molecular moiety that can be conjugated to an antibody. In particular embodiments, payloads are selected from the group consisting of therapeutic moieties and labelling moieties.

“Triple negative breast cancer” (TNBC) refers to a breast cancer characterized as estrogen receptor-negative, progesterone receptor-negative and human epidermal growth factor receptor-2-negative (HER2-negative). The TNBC can be BRCA1/2 wildtype or BRCA1/2 mutated. The determination of negative status of the estrogen, progesterone, and Her2/neu expression is readily determined by one of skill in the art, e.g., in accordance with the current accepted guidelines. For example, guidelines set forth by the American Society of Clinical Oncology (ASCO) and the College of American Pathologists (CAP) are widely accepted. The ASCO/CAP recommends testing by immunohistochemistry (IHC) or in situ hybridization (ISH) techniques. Further, a cancer is Her2 negative if a single test (or all tests) performed on a tumor specimen show: (a) IHC negative, IHC 1+ or IHC 0, or (b) ISH negative using single-probe ISH or dual-probe ISH. One of skill in the art would recognize that the triple negative cancer described herein does not include any cancer having an apparent histopathologic discordance as observed by the pathologist. Wolff, A C et al. J Clin Oncol. 2013 Nov. 1: 31 (31): 3997-4013. Cancer is ER-negative or PR-negative if <1% of tumor cell nuclei are immunoreactive in the presence of evidence that the sample can express ER or PR (positive intrinsic controls are seen).

As used herein, the term “inhibits growth” (e.g., referring to cells, such as tumor cells) is intended to include any measurable decrease in cell growth (e.g., tumor cell growth) when contacted with a TF antibody or antibody conjugate described herein, as compared to the growth of the same cells not in contact with a TF antibody. In some embodiments, growth may be inhibited by at least about 10%, 20%, 30%, 40%, 50%, 60%, 70%, 80%, 90%, 99%, or 100%. The decrease in cell growth can occur by a variety of mechanisms, including but not limited to antibody internalization, apoptosis, necrosis, and/or effector function-mediated activity.

In some chemical structures illustrated herein, certain substituents, chemical groups, and atoms are depicted with a curvy/wavy line (e.g.,

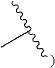

that intersects a bond or bonds to indicate the atom through which the substituents, chemical groups, and atoms are bonded. For example, in some structures, such as but not limited to

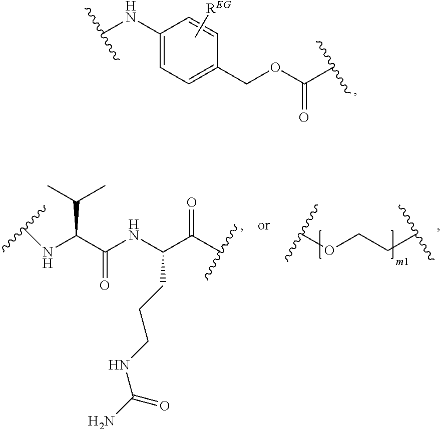

this curvy/wavy line indicates the atoms in the backbone of a conjugate or linker-payload to which the illustrated chemical entity is bonded. In some structures, such as but not limited to

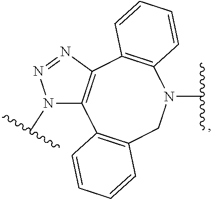 [SMILES: *N1Cc2ccccc2-c2c(nnn2*)-c2ccccc21]

this curvy/wavy line indicates the atoms in the antibody or antibody fragment as well as the atoms in the backbone of a conjugate or linker-payload to which the illustrated chemical entity is bonded.

Spiro compounds depicted with overlapping rings indicate that the rings can bond at any vertex. For instance, in the spiro group

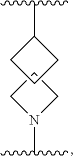

the two rings can bond at any of the three available vertex atoms in either ring.

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

“Alkoxy” and “alkoxyl,” refer to the group —OR″ where R″ is alkyl or cycloalkyl. Alkoxy groups include, in certain embodiments, methoxy, ethoxy, n-propoxy, isopropoxy, n-butoxy, tert-butoxy, sec-butoxy, n-pentoxy, n-hexoxy, 1,2-dimethylbutoxy, and the like.

The term “alkoxyamine,” as used herein, refers to the group -alkylene-O—NH₂, wherein alkylene is as defined herein. In some embodiments, alkoxyamine groups can react with aldehydes to form oxime residues. Examples of alkoxyamine groups include —CH₂CH₂—O—NH₂, —CH₂—O—NH₂, and —O—NH₂.

The term “alkyl,” as used herein, unless otherwise specified, refers to a saturated straight or branched hydrocarbon. In certain embodiments, the alkyl group is a primary, secondary, or tertiary hydrocarbon. In certain embodiments, the alkyl group includes one to ten carbon atoms (i.e., C₁ to C₁₀ alkyl). In certain embodiments, the alkyl is a lower alkyl, for example, C₁₋₆alkyl, and the like. In certain embodiments, the alkyl group is selected from the group consisting of methyl, ethyl, propyl, isopropyl, butyl, isobutyl, sec-butyl, t-butyl, pentyl, isopentyl, neopentyl, hexyl, isohexyl, 3-methylpentyl, 2,2-dimethylbutyl, and 2,3-dimethylbutyl. In certain embodiments, “substituted alkyl” refers to an alkyl substituted with, for example, one, two, or three groups independently selected from a halogen (e.g., fluoro (F), chloro (Cl), bromo (Br), or iodo (I)), alkyl, —CN, —NO₂, amido, —C(O)—, —C(S)—, ester, carbamate, alkenyl, alkynyl, cycloalkyl, heterocycloalkyl, dialkylamino, haloalkyl, hydroxyl, amino, alkylamino, and alkoxy. In some embodiments, alkyl is unsubstituted.

The term “alkylene,” as used herein, unless otherwise specified, refers to a divalent alkyl group, as defined herein. “Substituted alkylene” refers to an alkylene group substituted as described herein for alkyl. In some embodiments, alkylene is unsubstituted.

“Alkenyl” refers to an olefinically unsaturated hydrocarbon group, in certain embodiments, having up to about eleven carbon atoms or from two to six carbon atoms (e.g., “lower alkenyl”), which can be straight-chained or branched, and having at least one or from one to two sites of olefinic unsaturation. “Substituted alkenyl” refers to an alkenyl group substituted as described herein for alkyl.

“Alkenylene” refers to a divalent alkenyl as defined herein. Lower alkenylene is, for example, C₂-C₆-alkenylene.

“Alkynyl” refers to acetylenically unsaturated hydrocarbon groups, in certain embodiments, having up to about eleven carbon atoms or from two to six carbon atoms (e.g., “lower alkynyl”), which can be straight-chained or branched, and having at least one or from one to two sites of acetylenic unsaturation. Non-limiting examples of alkynyl groups include acetylene (—C≡CH), propargyl (—CH₂C≡CH), and the like. “Substituted alkynyl” refers to an alkynyl group substituted as described herein for alkyl.

“Alkynylene” refers to a divalent alkynyl as defined herein. Lower alkynylene is, for example, C₂-C₆-alkynylene.

“Amino” refers to —NH₂.

The term “alkylamino,” as used herein, and unless otherwise specified, refers to the group —NHR″ where R″ is, for example, C₁₋₁₀alkyl, C₂₋₁₀ alkenyl, C₂₋₁₀ alkynyl, C₃₋₁₂ carbocycle, 3- to 12-membered heterocycle, C₁₋₁₀ haloalkyl, and the like as defined herein. In certain embodiments, alkylamino is C₁₋₆alkylamino.

The term “dialkylamino,” as used herein, and unless otherwise specified, refers to the group —NR″R″ where each R″ is independently C₁₋₁₀alkyl, as defined herein. In certain embodiments, dialkylamino is, for example, di-C₁₋₆alkylamino, C₂₋₁₀ alkenyl, C₂₋₁₀ alkynyl, C₃₋₁₂ carbocycle, 3- to 12-membered heterocycle, C₁₋₁₀ haloalkyl, and the like.

The term “aryl,” as used herein, and unless otherwise specified, refers to phenyl, biphenyl, or naphthyl. The term includes both substituted and unsubstituted moieties. An aryl group can be substituted with any described moiety including, but not limited to, one or more moieties (e.g., in some embodiments one, two, or three moieties) selected from the group consisting of halogen (e.g., fluoro (F), chloro (Cl), bromo (Br), or iodo (I)), alkyl, haloalkyl, hydroxyl, amino, alkylamino, arylamino, alkoxy, aryloxy, nitro, cyano, sulfonic acid, sulfate, phosphonic acid, phosphate, and phosphonate, wherein each moiety is independently either unprotected, or protected as necessary, as would be appreciated by those skilled in the art (see, e.g., Greene, et al., Protective Groups in Organic Synthesis, John Wiley and Sons, Second Edition, 1991); and wherein the aryl in the arylamino and aryloxy substituents are not further substituted.

The term “arylamino,” as used herein, and unless otherwise specified, refers to an —NR′R″ group where R′ is hydrogen or C₁-C₆-alkyl; and R″ is aryl, as defined herein.

The term “arylene,” as used herein, and unless otherwise specified, refers to a divalent aryl group, as defined herein.

The term “aryloxy,” as used herein, and unless otherwise specified, refers to an —OR group where R is aryl, as defined herein.

“Alkarylene” refers to an arylene group, as defined herein, wherein the aryl ring is substituted with one or two alkyl groups. “Substituted alkarylene” refers to an alkarylene, as defined herein, where the arylene group is further substituted, as defined herein for aryl.

“Aralkylene” refers to a —CH₂-arylene-, -arylene-CH₂—, or —CH₂-arylene-CH₂— group, where arylene is as defined herein. “Substituted aralkylene” refers to an aralkylene, as defined herein, where the aralkylene group is substituted, as defined herein for aryl.

“Carboxyl” or “carboxy” refers to —C(O)OH or —COOH.

The term “cycloalkyl” as used herein, unless otherwise specified, refers to a saturated cyclic hydrocarbon. In certain embodiments, the cycloalkyl group may be saturated, and/or bridged, and/or non-bridged, and/or a fused bicyclic group and/or a spirocyclic bicyclic group. In certain embodiments, the cycloalkyl group includes three to ten carbon atoms (i.e., C₃ to C₁₀ cycloalkyl). In some embodiments, the cycloalkyl has from three to fifteen carbons (C₃₋₁₅), from three to ten carbons (C₃₋₁₀), from three to seven carbons (C₃₋₇), or from three to six carbons (C₃-C₆) (i.e., “lower cycloalkyl”). In certain embodiments, the cycloalkyl group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl. Exemplary “cycloalkyl” or “carbocycles” include cyclopentyl, cyclohexyl, cyclohexenyl, adamantyl, phenyl, indanyl, and naphthyl. “Cycloalkyl” or “carbocycle” includes 3- to 10-membered monocyclic rings, 6- to 12-membered bicyclic rings, and 6- to 12-membered bridged rings. Each ring of a bicyclic cycloalkyl or carbocycle may be selected from saturated, unsaturated, and aromatic rings. A bicyclic cycloalkyl or carbocycle includes any combination of saturated, unsaturated and aromatic bicyclic rings, as valence permits. A bicyclic cycloalkyl or carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Non-limiting examples of bridged bicyclic cycloalkyl or carbocycle groups include, but are not limited to, bicyclo[1.1.1]pentyl, bicyclo[2.1.1]hexyl, bicyclo[2.1.1]hexyl, bicyclo[3.1.1]heptyl, bicyclo[2.2.1]heptyl, bicyclo[3.2.1]octyl, bicyclo[2.2.2]octyl, bicyclo[3.3.1]nonyl, bicyclo[3.3.2]decyl, and 2-oxabicyclo[2.2.2]octyl. Non-limiting examples of spirocyclic cycloalkyl or carbocycle groups include, but are not limited to, spiro[3.3]heptyl, spiro[3.4]octyl, spiro[3.5]nonyl, spiro[3.6]decyl, spiro[4.4]nonyl, spiro[4.5]decyl, spiro[5.5]undecyl, spiro[5.6]dodecyl, and spiro[5.7]tridecyl.

The term “bicyclic ring system” includes 6-12 (e.g., 8-12 or 9-, 10-, or 11-) membered structures that form two rings, wherein the two rings have at least one atom in common (e.g., two atoms in common). Bicyclic rings can be fused, bridged, or spirocyclic. Bicyclic ring systems include bicycloaliphatics (e.g., bicycloalkyl or bicycloalkenyl), bicycloheteroaliphatics, bicyclic aryls, and bicyclic heteroaryls.

The term “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[1.1.1]pentyl, bicyclo[2.1.1]hexyl, bicyclo[2.1.1]hexyl, bicyclo[3.1.1]heptyl, bicyclo[2.2.1]heptyl, bicyclo[3.2.1]octyl, bicyclo[2.2.2]octyl, bicyclo[3.3.1]nonyl, bicyclo[3.3.2]decyl, azabicyclo[3.1.1]heptyl, 1-azabicyclo[2.2.1]heptyl, 2-azabicyclo[2.2.1]heptyl, 7-azabicyclo[2.2.1]heptyl, 1-azabicyclo[2.2.2]octyl, 3-azabicyclo[3.2.1]octyl, and 2-2-oxabicyclo[2.2.2]octyl, 6-azabicyclo[3.1.1]heptyl, 6-oxabicyclo[3.1.1]heptyl, 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl) alkyl, heterocycloalkyl, (heterocycloalkyl) alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl) carbonylamino, arylcarbonylamino, aralkylcarbonylamino, (heterocycloalkyl) carbonylamino, (heterocycloalkylalkyl) carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

The term “spiro bicyclic ring system” refers to a bicyclic heterocyclicalipahtic ring system or bicyclic cycloaliphatic ring system in which 2 or 3 rings are linked together by one common atom. Spiro compounds depicted with overlapping rings indicate that the rings can bond at any vertex. For instance, in the spiro group

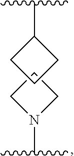

the two rings can bond at any of the three available vertex atoms in either ring.

The term “cycloalkylene,” as used herein refers to a divalent cycloalkyl group, as defined herein. In certain embodiments, the cycloalkylene group is cyclopropylene

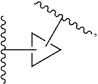

cyclobutylene

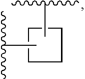

cyclopentylene

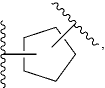

cyclohexylene

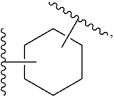

cycloheptylene

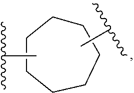

and the like. Lower cycloalkylene refers to a C₃-C₆-cycloalkylene.

The term “cycloalkylalkyl,” as used herein, unless otherwise specified, refers to an alkyl group, as defined herein, substituted with one or two cycloalkyl, as defined herein.

The term “ester,” as used herein, refers to —C(O)OR or —COOR where R is alkyl, as defined herein.

The term “fluorene” as used herein refers to

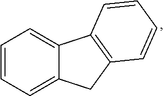 [SMILES: c1ccc2c(c1)Cc1ccccc1-2 ; ","]

wherein any one or more carbons bearing one or more hydrogens can be substituted with a chemical functional group as described herein.

The term “haloalkyl” refers to an alkyl group, as defined herein, substituted with one or more halogen atoms (e.g., in some embodiments one, two, three, four, or five) which are independently selected.

The term “heteroalkyl” refers to an alkyl, as defined herein, in which one or more carbon atoms are replaced by heteroatoms. As used herein, “heteroalkenyl” refers to an alkenyl, as defined herein, in which one or more carbon atoms are replaced by heteroatoms. As used herein, “heteroalkynyl” refers to an alkynyl, as defined herein, in which one or more carbon atoms are replaced by heteroatoms. Suitable heteroatoms include, but are not limited to, nitrogen (N), oxygen (O), and sulfur(S) atoms. Heteroalkyl, heteroalkenyl, and heteroalkynyl are optionally substituted. Examples of heteroalkyl moieties include, but are not limited to, aminoalkyl, sulfonylalkyl, and sulfinylalkyl. Examples of heteroalkyl moieties also include, but are not limited to, methylamino, methylsulfonyl, and methylsulfinyl. “Substituted heteroalkyl” refers to heteroalkyl substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a heteroalkyl group may comprise one, two, three, or four heteroatoms. Those of skill in the art will recognize that a 4-membered heteroalkyl may generally comprise one or two heteroatoms, a 5- or 6-membered heteroalkyl may generally comprise one, two, or three heteroatoms, and a 7- to 10-membered heteroalkyl may generally comprise one, two, three, or four heteroatoms.

The term “heteroalkylene,” as used herein, refers to a divalent heteroalkyl, as defined herein. “Substituted heteroalkylene” refers to a divalent heteroalkyl, as defined herein, substituted as described for heteroalkyl.

The term “heterocycloalkyl” refers to a monovalent, monocyclic, or multicyclic non-aromatic ring system, wherein one or more of the ring atoms are heteroatoms independently selected from oxygen (O), sulfur(S), and nitrogen (N) (e.g., where the nitrogen or sulfur atoms may be optionally oxidized, and the nitrogen atoms may be optionally quaternized) and the remaining ring atoms of the non-aromatic ring are carbon atoms. In certain embodiments, heterocycloalkyl is a monovalent, monocyclic, or multicyclic fully-saturated ring system. In certain embodiments, the heterocycloalkyl group has from three to twenty, from three to fifteen, from three to ten, from three to eight, from four to seven, from four to eleven, or from five to six ring atoms. The heterocycloalkyl may be attached to a core structure at any heteroatom or carbon atom which results in the creation of a stable compound. In certain embodiments, the heterocycloalkyl is a monocyclic, bicyclic, tricyclic, or tetracyclic ring system, which may include a fused, and/or bridged bicyclic group, and/or a spirocyclic bicyclic group ring system and in which the nitrogen or sulfur atoms may be optionally oxidized, and/or the nitrogen atoms may be optionally quaternized. In some embodiments, heterocycloalkyl radicals include, but are not limited to, 2,5-diazabicyclo[2.2.2]octanyl, decahydroisoquinolinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, heterocycloalkyl may also be optionally substituted as described herein. In certain embodiments, heterocycloalkyl is substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a heterocycloalkyl group may comprise one, two, three, or four heteroatoms. Those of skill in the art will recognize that a 4-membered heterocycloalkyl may generally comprise one or two heteroatoms, a 5- or 6-membered heterocycloalkyl may generally comprise one, two, or three heteroatoms, and a 7- to 10-membered heterocycloalkyl may generally comprise one, two, three, or four heteroatoms. In some embodiments, “heterocycloalkyl” or “heterocycle” radicals include, but are not limited to, 2,5-diazabicyclo[2.2.2]octanyl, decahydroisoquinolinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. Non-limiting examples of bridged heterocycloalkyl or heterocycle groups include, but are not limited to, 6-azabicyclo[3.1.1]heptyl, 6-azabicyclo[3.1.1]heptyl, 1-azabicyclo[2.2.1]heptyl, 2-azabicyclo[2.2.1]heptyl, 7-azabicyclo[2.2.1]heptyl, 1-azabicyclo[2.2.2]octyl, 3-azabicyclo[3.2.1]octyl, and 2-oxabicyclo[3.1.1]heptyl, 2,6-dioxa-tricyclo[3.3.1.0^{3,7}]nonyl. Non-limiting examples of spirocyclic heterocycloalkyl or heterocycle groups include, but are not limited to, 2,8-diazaspiro[4.5]decyl; 2,7-diazaspiro[3.5]nonyl; 3,9-diazaspiro[5.5]undecyl; 3-azaspiro[5.5]undecyl; 2-oxa-6-azaspiro[3.4]octyl; 2-oxa-9-azaspiro[5.5]undecyl; 3-oxa-9-azaspiro[5.5]undecyl; 7-azaspiro[3.5]nonyl; 2-azaspiro[3.5]nonyl; 7-oxaspiro[3.5]nonyl; and, 2-oxaspiro[3.5]nonyl.

“Heterocycloalkylene” refers to a divalent heterocycloalkyl as defined herein.

The term “heteroaryl” refers to a monovalent, monocyclic aromatic group and/or multicyclic aromatic group, wherein at least one aromatic ring contains one or more heteroatoms independently selected from oxygen, sulfur, and nitrogen within the ring. Each ring of a heteroaryl group can contain one or two oxygen atoms, one or two sulfur atoms, and/or one to four nitrogen atoms, provided that the total number of heteroatoms in each ring is four or less and each ring contains at least one carbon atom. In certain embodiments, the heteroaryl has from five to twenty, from five to fifteen, or from five to ten ring atoms. A heteroaryl may be attached to the rest of the molecule via a nitrogen or a carbon atom. In some embodiments, monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, triazolyl, thiadiazolyl, thiazolyl, thienyl, tetrazolyl, and triazinyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted as described herein. “Substituted heteroaryl” is a heteroaryl substituted as defined for aryl.

The term “heteroarylene” refers to a divalent heteroaryl group, as defined herein. “Substituted heteroarylene” is a heteroarylene substituted as defined for aryl.

The term “protecting group,” as used herein, and unless otherwise specified, refers to a group that is added to an oxygen, nitrogen, or phosphorus atom to prevent further reaction at the (protected) oxygen, nitrogen, or phosphorus, or for other purposes. A wide variety of oxygen and nitrogen protecting groups are known to those skilled in the art of organic synthesis (see, e.g., Greene, et al., Protective Groups in Organic Synthesis, John Wiley and Sons, Fourth Edition, 2006, which is incorporated herein by reference in its entirety).

“Pharmaceutically acceptable salt” refers to any salt of a compound provided herein which retains its biological properties and which is not toxic or otherwise undesirable for pharmaceutical use. Such salts may be derived from a variety of organic and inorganic counter-ions well known in the art. Such salts include, but are not limited to (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl) benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, glutamic, hydroxynaphthoic, salicylic, stearic, cyclohexylsulfamic, quinic, and muconic acids, and the like; or (2) salts formed when an acidic proton present in the parent compound either (a) is replaced by a metal ion, for example, an alkali metal ion, an alkaline earth ion, or an aluminum ion, or alkali metal or alkaline earth metal hydroxides, such as sodium, potassium, calcium, magnesium, aluminum, lithium, zinc, and barium hydroxide, or ammonia; or (b) coordinates with an organic base, such as aliphatic, alicyclic, or aromatic organic amines, including, without limitation, ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, N-procaine, N-benzylphenethylamine, methylglucamine piperazine, tris(hydroxymethyl)-aminomethane, tetramethylammonium hydroxide, and the like.

Pharmaceutically acceptable salts further include, by way of example and without limitation, sodium, potassium, calcium, magnesium, ammonium, and tetraalkylammonium salts, and the like, and when the compound contains a basic functionality, salts of non-toxic organic or inorganic acids, such as hydrohalides, for example, hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl) benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, glutamate, hydroxynaphthoate, salicylate, stearate, cyclohexylsulfamate, quinate, muconate, and the like.

The term “substantially free of” or “substantially in the absence of” with respect to a composition refers to a composition that includes at least 85% or 90% by weight, in certain embodiments 95%, 98%, 99%, or 100% by weight; or in certain embodiments, 95%, 98%, 99%, or 100% of the designated enantiomer or diastereomer of a compound. In certain embodiments, in the methods and compounds provided herein, the compounds are substantially free of one of two enantiomers. In certain embodiments, in the methods and compounds provided herein, the compounds are substantially free of one of two diastereomers. In certain embodiments, in the methods and compounds provided herein, the compounds are substantially free of enantiomers (i.e., the compounds are not a racemic or 50:50 mixture of compounds).

Similarly, the term “isolated” with respect to a composition refers to a composition that includes at least 85%, 90%, 95%, 98%, or 99% to 100% by weight, of the compound, the remainder comprising other chemical species, enantiomers, or diastereomers.

“Solvate” refers to a compound provided herein, or a salt thereof, that further includes a stoichiometric or non-stoichiometric amount of solvent bound by non-covalent intermolecular forces. Where the solvent is water, the solvate is a hydrate.

“Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as hydrogen (H), the position is understood to have hydrogen at its natural isotopic composition.

“Isotopic enrichment” refers to the percentage of incorporation of an amount of a specific isotope at a given atom in a molecule in the place of that atom's natural isotopic abundance. For example, deuterium (D) enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, “alkyl,” “alkylene,” “alkylamino,” “dialkylamino,” “cycloalkyl,” “aryl,” “arylene,” “alkoxy,” “amino,” “carboxyl,” “heterocycloalkyl,” “heteroaryl,” “heteroarylene,” “carboxyl,” and “amino acid” groups optionally comprise deuterium (D) at one or more positions where hydrogen (H) atoms are present, and wherein the deuterium composition of the atom or atoms is other than the natural isotopic composition.

Also as used herein, “alkyl,” “alkylene,” “alkylamino,” “dialkylamino,” “cycloalkyl,” “aryl,” “arylene,” “alkoxy,” “amino,” “carboxyl,” “heterocycloalkyl,” “heteroaryl,” “heteroarylene,” “carboxyl,” and “amino acid” groups optionally comprise carbon-13 (¹³C) at an amount other than the natural isotopic composition.

The term “macromolecule” or “macromolecular moiety” refers to a protein, peptide, antibody, nucleic acid, carbohydrate, or other large molecule composed of polymerized monomers. They include peptides of two or more residues, or ten or more residues. In certain embodiments, a macromolecule is at least 1000 Da in mass. In certain embodiments, a macromolecule has at least 1000 atoms. In certain embodiments, a macromolecule can be modified. For instance, a protein, peptide, or antibody can be modified with one or more carbohydrates and/or small molecule therapeutic compounds.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V), and the less common pyrrolysine and selenocysteine. Natural amino acids also include citrulline. Naturally encoded amino acids include post-translational variants of the twenty-two naturally occurring amino acids such as prenylated amino acids, isoprenylated amino acids, myrisoylated amino acids, palmitoylated amino acids, N-linked glycosylated amino acids, O-linked glycosylated amino acids, phosphorylated amino acids, and acylated amino acids. The term “amino acid” also includes non-natural (or unnatural) or synthetic α-, β-, γ-, or δ-amino acids, and includes, but is not limited to, amino acids found in proteins, i.e., glycine, alanine, valine, leucine, isoleucine, methionine, phenylalanine, tryptophan, proline, serine, threonine, cysteine, tyrosine, asparagine, glutamine, aspartate, glutamate, lysine, arginine, and histidine. In certain embodiments, the amino acid is in the L-configuration. In certain embodiments, the amino acid is in the D-configuration. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl, or β-histidinyl. Unnatural amino acids are not proteinogenic amino acids, or post-translationally modified variants thereof. In particular, the term unnatural amino acid refers to an amino acid that is not one of the twenty common amino acids or pyrrolysine or selenocysteine, or post-translationally modified variants thereof.

The term “conjugate” or “antibody conjugate” refers to a compound or drug moiety described herein linked to one or more macromolecular moieties. The macromolecular moiety is as defined herein or is any macromolecule deemed suitable to the person of skill in the art. The compound or drug moiety can be any compound or drug moiety described herein. The compound or drug moiety can be directly linked to the macromolecular moiety via a covalent bond, or the compound or drug moiety can be linked to the macromolecular moiety indirectly via a linker. Typically, the linker is covalently bonded to the macromolecular moiety and also covalently bonded to the compound or drug moiety.

“pAMF,” “pAMF residue,” or “pAMF mutation” refers to a variant phenylalanine residue (i.e., para-azidomethyl-L-phenylalanine) added or substituted into a polypeptide.

The term “linker” refers to a molecular moiety that is capable of forming at least two covalent bonds. Typically, a linker is capable of forming at least one covalent bond to a macromolecular moiety and at least another covalent bond to a compound or drug moiety. In certain embodiments, a linker can form more than one covalent bond to a macromolecular moiety. In certain embodiments, a linker can form more than one covalent bond to a compound or drug moiety or can form covalent bonds to more than one compound or drug moiety. After a linker forms a bond to a macromolecular moiety, or a compound or drug moiety, or both, the remaining structure (i.e. the residue of the linker (“linker residue”) after one or more covalent bonds are formed) may still be referred to as a “linker” herein. The term “linker precursor” refers to a linker having one or more reactive groups capable of forming a covalent bond with a macromolecule, or compound or drug moiety, or both. A person of ordinary skill in the art, given the context of how the term linker is used, would understand whether “linker” means linker precursor with one reactive group, a linker precursor with more than one reactive groups, a linker residue which is covalently bonded to the macromolecule, a linker residue which is covalently bonded to a compound or drug moiety, and/or a linker residue which is covalently bonded to the macromolecule and is covalently bonded to a compound or drug moiety. In some embodiments, the linker is a cleavable linker. For example, a cleavable linker can be one that is released by a bio-labile or enzymatic function, which may or may not be engineered. In some embodiments, the linker is a non-cleavable linker. For example, a non-cleavable linker can be one that is released upon degradation of the macromolecular moiety.

As used herein, term “EC₅₀” refers to a dosage, concentration, or amount of a particular test compound that elicits a dose-dependent response at 50% of maximal expression of a particular response that is induced, provoked, or potentiated by the particular test compound.

As used herein, and unless otherwise specified, the term “IC₅₀” refers to an amount, concentration, or dosage of a particular test compound that achieves a 50% inhibition of a maximal response in an assay that measures such response.

As used herein, the terms “subject” and “patient” are used interchangeably. The terms “subject” and “subjects” refer to an animal, such as a mammal including a non-primate (e.g., a cow, pig, horse, cat, dog, rat, mouse, camel, avian, goat, and sheep) and a primate (e.g., a monkey, such as a cynomolgous monkey, a chimpanzee, and a human), and in certain embodiments, a human. In certain embodiments, the subject is a farm animal (e.g., a horse, a cow, a pig, etc.) or a pet (e.g., a dog or a cat). In certain embodiments, the subject is a human. In some embodiments, the subject has a disease that can be treated or diagnosed with an antibody or antibody conjugate provided herein. In some embodiments, the disease is gastric carcinoma, colorectal carcinoma, renal cell carcinoma, cervical carcinoma, non-small cell lung carcinoma, ovarian cancer, breast cancer, triple-negative breast cancer, endometrial cancer, prostate cancer, and/or a cancer of epithelial origin.

As used herein, the terms “therapeutic agent” and “therapeutic agents” refer to any agent(s) which can be used in the treatment or prevention of a disorder or one or more symptoms thereof. In certain embodiments, the term “therapeutic agent” includes an antibody or antibody conjugate provided herein. In certain embodiments, a therapeutic agent is an agent which is known to be useful for, or has been or is currently being used for the treatment or prevention of a disorder or one or more symptoms thereof.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder, or delaying or preventing recurrence of the disease or disorder. In yet another embodiment, “treating” or “treatment” includes the reduction or elimination of either the disease or disorder, or retarding the progression of the disease or disorder or of one or more symptoms of the disease or disorder, or reducing the severity of the disease or disorder or of one or more symptoms of the disease or disorder.

As used herein, the terms “prophylactic agent” and “prophylactic agents” as used refer to any agent(s) which can be used in the prevention of a disorder or one or more symptoms thereof. In certain embodiments, the term “prophylactic agent” includes a compound, drug moiety, or conjugate provided herein. In certain other embodiments, the term “prophylactic agent” does not refer a compound, drug moiety, or conjugate provided herein. For example, a prophylactic agent is an agent which is known to be useful for, or has been or is currently being used to prevent or impede the onset, development, progression, and/or severity of a disorder.

As used herein, the phrase “prophylactically effective amount” refers to the amount of a therapy (e.g., prophylactic agent) which is sufficient to result in the prevention or reduction of the development, recurrence, or onset of one or more symptoms associated with a disorder or to enhance or improve the prophylactic effect(s) of another therapy (e.g., another prophylactic agent).

In some chemical structures illustrated herein, certain substituents, chemical groups, and atoms are depicted with a curvy/wavy/wiggly line (e.g.,

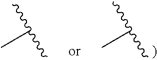

that intersects a bond or bonds to indicate the atom through which the substituents, chemical groups, and atoms are bonded. For example, in some structures, such as but not limited to,

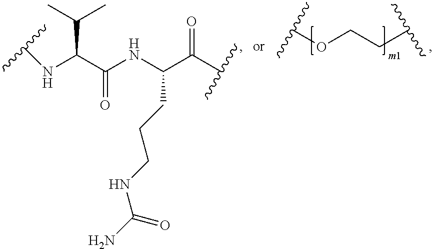

this curvy/wavy/wiggly line indicates the atoms in the backbone of a conjugate, compound, or drug moiety structure to which the illustrated chemical entity is bonded. In some structures, such as but not limited to

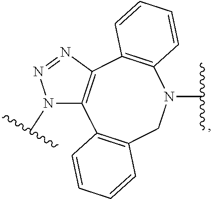 [SMILES: *N1Cc2ccccc2-c2c(nnn2*)-c2ccccc21]

this curvy/wavy/wiggly line indicates the atoms in the macromolecule as well as the atoms in the backbone of a conjugate, compound, or drug moiety structure to which the illustrated chemical entity is bonded.

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains. For example, the group,

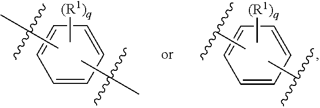

wherein subscript q is an integer from zero to four and in which the positions of substituent R¹ are described generically, i.e., not directly attached to any vertex of the bond line structure, i.e., specific ring carbon atom, includes the following, non-limiting examples of groups in which the substituent R¹ is bonded to a specific ring carbon atom:

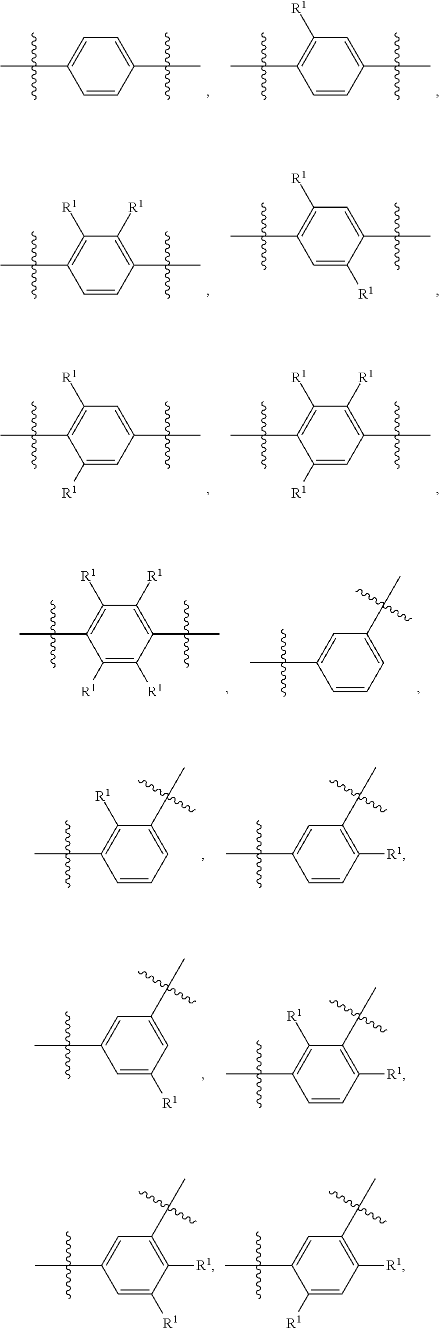

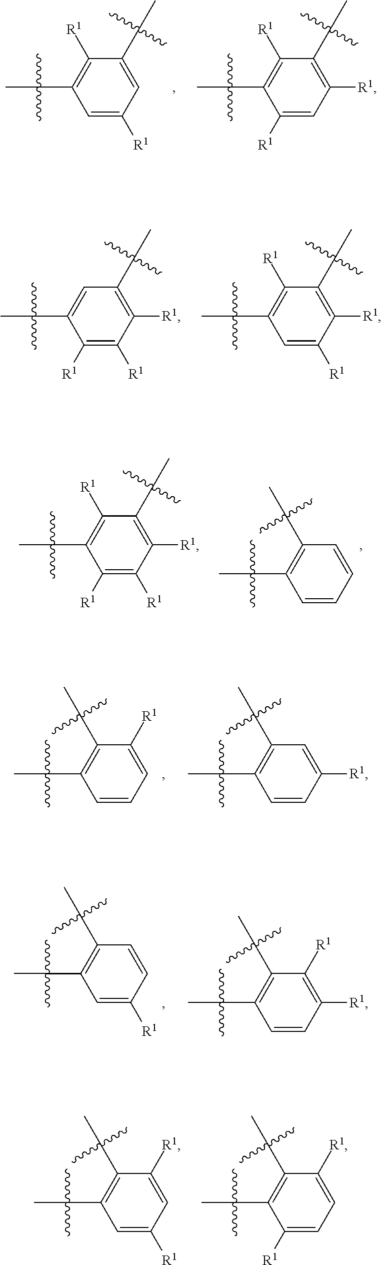

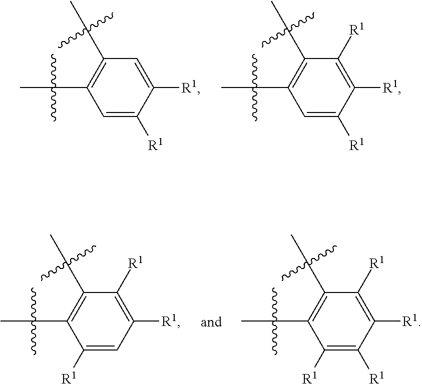

The term “carbocycle” as used herein, unless otherwise specified, refers to a saturated, unsaturated, or aromatic ring in which atom of the ring is carbon. In certain embodiments, the carbocycle group may be saturated, and/or bridged, and/or non-bridged, and/or a fused bicyclic group, and/or a spirocyclic bicyclic group. In certain embodiments, the carbocycle group includes three to ten carbon atoms (i.e., C₃ to C₁₀ carbocycle). In some embodiments, the carbocycle has from three to fifteen carbons (C₃₋₁₅), from three to ten carbons (C₃₋₁₀), from three to seven carbons (C₃₋₇), or from three to six carbons (C₃-C₆). In certain embodiments, the carbocycle group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl.

The term “heterocycle” refers to a saturated, unsaturated or aromatic ring comprising one or more heteroatoms. Exemplary heteroatoms include N, O, Si, P, B, and S atoms where the nitrogen or sulfur atoms may be optionally oxidized, and the nitrogen atoms may be optionally quaternized and the remaining ring atoms of the non-aromatic ring are carbon atoms. Heterocycles include 3- to 10-membered monocyclic rings, 6- to 12-membered bicyclic rings, and 6- to 12-membered bridged rings. In certain embodiments, heterocycle is a monovalent, monocyclic, or multicyclic fully-saturated ring system. In certain embodiments, the heterocycloalkyl or “heterocycle” group may be unsaturated, and/or bridged, and/or non-bridged, and/or a fused bicyclic group, and/or a spirocyclic bicyclic group.

The term “site-specific” refers to a modification of a polypeptide at a predetermined sequence location in the polypeptide. The modification is at a single, predictable residue of the polypeptide with little or no variation. In particular embodiments, a modified amino acid is introduced at that sequence location, for instance recombinantly or synthetically. Similarly, a moiety can be “site-specifically” linked to a residue at a particular sequence location in the polypeptide. In certain embodiments, a polypeptide can comprise more than one site-specific modification.

As used herein, the term “therapeutically effective amount” or “effective amount” refers to an amount of an antibody, antibody conjugate, or composition that when administered to a subject is effective to treat a disease or disorder. In some embodiments, a therapeutically effective amount or effective amount refers to an amount of an antibody, antibody conjugate, or composition that when administered to a subject is effective to prevent or ameliorate a disease or the progression of the disease, or result in amelioration of symptoms. A “therapeutically effective amount” can vary depending on, inter alia, the compound, the disease or disorder and its severity, and the age, weight, etc., of the subject to be treated.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has a disease that can be treated or diagnosed with an antibody provided herein. In some embodiments, the disease is gastric carcinoma, colorectal carcinoma, renal cell carcinoma, cervical carcinoma, non-small cell lung carcinoma, ovarian cancer, prostate cancer, and/or a cancer of epithelial origin.

### 2. Antibodies and Antibody Specificity

The present disclosure provides antibodies as described here that selectively bind to TF. Provided herein are antibodies that selectively bind human TF. In some aspects, the antibody selectively binds to the extracellular domain of human TF.

Also provided herein are conjugates that comprise antibodies that selectively bind human TF. In some aspects, the antibody of the conjugate selectively binds to the extracellular domain of human TF.

In some embodiments, the antibody binds to a homolog of human TF. In some aspects, the antibody binds to a homolog of human TF from a species selected from monkeys, mice, dogs, cats, rats, cows, horses, goats and sheep. In some aspects, the homolog is a cynomolgus monkey homolog.

In some embodiments, the antibody has one or more CDRs having particular lengths, in terms of the number of amino acid residues. In some embodiments, the Chothia CDR-H1 of the antibody is 6, 7, or 8 residues in length. In some embodiments, the Kabat CDR-H1 of the antibody is 4, 5, or 6 residues in length. In some embodiments, the Chothia CDR-H2 of the antibody is 5, 6, or 7 residues in length. In some embodiments, the Kabat CDR-H2 of the antibody is 16, 17, or 18 residues in length. In some embodiments, the Kabat/Chothia CDR-H3 of the antibody is 7, 8, 9, 10, 11, 12, or 13 residues in length.

In some aspects, the Kabat/Chothia CDR-L1 of the antibody is 10, 11, 12, 13, 14, 15, or 16 residues in length. In some aspects, the Kabat/Chothia CDR-L2 of the antibody is 6, 7, or 8 residues in length. In some aspects, the Kabat/Chothia CDR-L3 of the antibody is 8, 9, or 10 residues in length.

In some embodiments, the antibody comprises a light chain. In some aspects, the light chain is a kappa light chain. In some aspects, the light chain is a lambda light chain.

In some embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the scFv-Fc fragment comprises a constant region wherein the constant region comprises SEQ ID NO: 3068. The constant region in SEQ ID NO: 3068 differs from the human IgG1 constant region of SEQ ID NO: 3062 in several respects. First, the sequence in SEQ ID NO: 3068 comprises the linker AAGSDQEPKSS (SEQ ID NO: 3071). SEQ ID NO: 3068 also does not comprise the CH1 domain of the IgG1 constant region. SEQ ID NO: 3068 further comprises a C220S (EU numbering system) mutation, which removes an unpaired cysteine reside that is not needed when the light chain constant region is not present (e.g., in an scFv-Fc format). SEQ ID NO: 3068 further comprises two, optional, P to S mutations (P230S and P238S by the EU numbering system). Either or both of these serine residues can be reverted to the naturally occurring proline residues. Finally, SEQ ID NO: 3068 comprises an aspartic acid (D) residue at EU position 356 and a leucine (L) residue at EU position 358. In contrast, SEQ ID NO: 3062 comprises glutamic acid (E) in EU position 356 and methionine (M) in EU position 358. In some embodiments, the antibodies provided herein comprise constant regions comprising D356/L358, E356/M358, D356/M358, or E356/L358 (EU numbering). However, a skilled person will recognize that the antibodies provide herein may comprise any suitable constant region and that the constant region sequences provided herein are for illustrative purposes.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

In some embodiments, the antibody is an affinity matured antibody. In some aspects, the antibody is an affinity matured antibody derived from an illustrative sequence provided in this disclosure.

The antibodies provided herein may be useful for the treatment of a variety of diseases and conditions including cancers. In some embodiments, the antibodies provided herein may be useful for the treatment of cancers of solid tumors. For example, the antibodies provided herein can be useful for the treatment of colorectal cancer.

### 2.2 V_{H} Sequences Comprising Illustrative CDRs

In some embodiments, the antibody comprises a V_{H} sequence comprising one or more CDR-H sequences comprising, consisting of, or consisting essentially of one or more illustrative CDR-H sequences provided in this disclosure, and variants thereof. In some embodiments, the CDR-H sequences comprise, consist of, or consist essentially of one or more CDR-H sequences provided in a V_{H} sequence selected from SEQ ID NOs: 2625-2949.

### 2.2.1. V_{H} Sequences Comprising Illustrative Kabat CDRs

In some embodiments, the antibody comprises a V_{H} sequence comprising one or more Kabat CDR-H sequences comprising, consisting of, or consisting essentially of one or more illustrative Kabat CDR-H sequences provided in this disclosure, and variants thereof.

### 2.2.1.1. Kabat CDR-H1+Kabat CDR-H2+Kabat CDR-H3

In some embodiments, the antibody comprises a V_{H} sequence comprising a Kabat CDR-H1 sequence comprising, consisting of, or consisting essentially of a sequence selected from SEQ ID NOs: 430, 439, 442, 454-541, 543, 549, 554, 560, 562, and 563, a Kabat CDR-H2 sequence comprising, consisting of, or consisting essentially of a sequence selected from SEQ ID NOs: 1080, 1089, 1092, 1104-1191, 1193, 1199, 1204, 1200, 1212, and 1213, and a Kabat CDR-H3 sequence comprising, consisting of, or consisting essentially of a sequence selected from SEQ ID NOs: 1730, 1739, 1742, 1754-1841, 1843, 1849, 1854, 1860, 1862, and 1863. In some aspects, the Kabat CDR-H1 sequence, Kabat CDR-H2 sequence, and Kabat CDR-H3 sequence are all from a single illustrative V_{H} sequence provided in this disclosure. For example, in some aspects, the Kabat CDR-H1, Kabat CDR-H2, and Kabat CDR-H3 are all from a single illustrative V_{H} sequence selected from SEQ ID NOs: 2727, 2736, 2739, 2751-2838, 2840, 2846, 2851, 2857, 2859, and 2860.

### 2.2.1.2. Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some aspects, the Kabat CDR-H3 sequence comprises, consists of, or consists essentially of a variant of an illustrative Kabat CDR-H3 sequence provided in this disclosure. In some aspects, the Kabat CDR-H3 sequence comprises, consists of, or consists essentially of a sequence having at least 70%, 75%, 80%, 85%, 90%, 95%, 96%, 97%, 98%, or 99% identity with any of the illustrative Kabat CDR-H3 sequences provided in this disclosure. In some aspects, the Kabat CDR-H3 sequence comprises, consists of, or consists essentially of any of the illustrative Kabat CDR-H3 sequences provided in this disclosure, with 1, 2, or 3 amino acid substitutions. In some aspects, the amino acid substitutions are conservative amino acid substitutions.

In some aspects, the Kabat CDR-H2 sequence comprises, consists of, or consists essentially of a variant of an illustrative Kabat CDR-H2 sequence provided in this disclosure. In some aspects, the Kabat CDR-H2 sequence comprises, consists of, or consists essentially of a sequence having at least 70%, 75%, 80%, 85%, 90%, 95%, 96%, 97%, 98%, or 99% identity with any of the illustrative Kabat CDR-H2 sequences provided in this disclosure. In some aspects, the Kabat CDR-H2 sequence comprises, consists of, or consists essentially of any of the illustrative Kabat CDR-H2 sequences provided in this disclosure, with 1, 2, or 3 amino acid substitutions. In some aspects, the amino acid substitutions are conservative amino acid substitutions.

In some aspects, the Kabat CDR-H1 sequence comprises, consists of, or consists essentially of a variant of an illustrative Kabat CDR-H1 sequence provided in this disclosure. In some aspects, the Kabat CDR-H1 sequence comprises, consists of, or consists essentially of a sequence having at least 70%, 75%, 80%, 85%, 90%, 95%, 96%, 97%, 98%, or 99% identity with any of the illustrative Kabat CDR-H1 sequences provided in this disclosure. In some aspects, the Kabat CDR-H1 sequence comprises, consists of, or consists essentially of any of the illustrative Kabat CDR-H1 sequences provided in this disclosure, with 1, 2, or 3 amino acid substitutions. In some aspects, the amino acid substitutions are conservative amino acid substitutions.

### 2.2.2. V_{H} Sequences Comprising Illustrative Chothia CDRs

In some embodiments, the antibody comprises a V_{H} sequence comprising one or more Chothia CDR-H sequences comprising, consisting of, or consisting essentially of one or more illustrative Chothia CDR-H sequences provided in this disclosure, and variants thereof.

### 2.2.2.1. Chothia CDR-H1+Chothia CDR-H2+Chothia CDR-H3

In some embodiments, the antibody comprises a V_{H} sequence comprising a Chothia CDR-H1 sequence comprising, consisting of, or consisting essentially of a sequence selected from SEQ ID NOs: 105, 114, 117, 129-216, 218, 224, 229, 235, 237, and 238, a Chothia CDR-H2 sequence comprising, consisting of, or consisting essentially of a sequence selected from SEQ ID NOs: 755, 764, 767, 779-866, 868, 874, 879, 885, 887, and 888, and a Chothia CDR-H3 sequence comprising, consisting of, or consisting essentially of a sequence selected from SEQ ID NOs: 1405, 1414, 1417, 1429-1516, 1518, 1524, 1529, 1535, 1537, and 1538. In some aspects, the Chothia CDR-H1 sequence, Chothia CDR-H2 sequence, and Chothia CDR-H3 sequence are all from a single illustrative V_{H} sequence provided in this disclosure. For example, in some aspects, the Chothia CDR-H1, Chothia CDR-H2, and Chothia CDR-H3 are all from a single illustrative V_{H} sequence selected from SEQ ID NOs: 2727, 2736, 2739, 2751-2838, 2840, 2846, 2851, 2857, 2859, and 2860.

### 2.2.2.2. Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some aspects, the Chothia CDR-H3 sequence comprises, consists of, or consists essentially of a variant of an illustrative Chothia CDR-H3 sequence provided in this disclosure. In some aspects, the Chothia CDR-H3 sequence comprises, consists of, or consists essentially of a sequence having at least 70%, 75%, 80%, 85%, 90%, 95%, 96%, 97%, 98%, or 99% identity with any of the illustrative Chothia CDR-H3 sequences provided in this disclosure. In some aspects, the Chothia CDR-H3 sequence comprises, consists of, or consists essentially of any of the illustrative Chothia CDR-H3 sequences provided in this disclosure, with 1, 2, or 3 amino acid substitutions. In some aspects, the amino acid substitutions are conservative amino acid substitutions.

In some aspects, the Chothia CDR-H2 sequence comprises, consists of, or consists essentially of a variant of an illustrative Chothia CDR-H2 sequence provided in this disclosure. In some aspects, the Chothia CDR-H2 sequence comprises, consists of, or consists essentially of a sequence having at least 70%, 75%, 80%, 85%, 90%, 95%, 96%, 97%, 98%, or 99% identity with any of the illustrative Chothia CDR-H2 sequences provided in this disclosure. In some aspects, the Chothia CDR-H2 sequence comprises, consists of, or consists essentially of any of the illustrative Chothia CDR-H2 sequences provided in this disclosure, with 1, 2, or 3 amino acid substitutions. In some aspects, the amino acid substitutions are conservative amino acid substitutions.

In some aspects, the Chothia CDR-H1 sequence comprises, consists of, or consists essentially of a variant of an illustrative Chothia CDR-H1 sequence provided in this disclosure. In some aspects, the Chothia CDR-H1 sequence comprises, consists of, or consists essentially of a sequence having at least 70%, 75%, 80%, 85%, 90%, 95%, 96%, 97%, 98%, or 99% identity with any of the illustrative Chothia CDR-H1 sequences provided in this disclosure. In some aspects, the Chothia CDR-H1 sequence comprises, consists of, or consists essentially of any of the illustrative Chothia CDR-H1 sequences provided in this disclosure, with 1, 2, or 3 amino acid substitutions. In some aspects, the amino acid substitutions are conservative amino acid substitutions.

### 2.3. V_{H} Sequences

In some embodiments, the antibody comprises, consists of, or consists essentially of a V_{H} sequence of an scFv sequence provided in SEQ ID NOs: 3073 and 3079. In some embodiments, the antibody comprises, consists of, or consists essentially of a V_{H} sequence provided in SEQ ID NOs.: 2727, 2736, 2739, 2751-2838, 2840, 2846, 2851, 2857, 2859, and 2860.

In some embodiments, the antibody comprises a V_{H} sequence comprising, consisting of, or consisting essentially of a sequence selected from SEQ ID NOs: 2727, 2736, 2739, 2751-2838, 2840, 2846, 2851, 2857, 2859, and 2860.

In some aspects, the antibody comprises a V_{H} sequence comprising, consisting of, or consisting essentially of SEQ ID NO:2727. In some aspects, the antibody comprises a V_{H} sequence comprising, consisting of, or consisting essentially of SEQ ID NO:2736. In some aspects, the antibody comprises a V_{H} sequence comprising, consisting of, or consisting essentially of SEQ ID NO:2739.

In some aspects, the antibody comprises a V_{H} sequence comprising, consisting of, or consisting essentially of SEQ ID NO:2751. In some aspects, the antibody comprises a V_{H} sequence comprising, consisting of, or consisting essentially of SEQ ID NO:2752. In some aspects, the antibody comprises a V_{H} sequence comprising, consisting of, or consisting essentially of SEQ ID NO:2753. In some aspects, the antibody comprises a V_{H} sequence comprising, consisting of, or consisting essentially of SEQ ID NO:2754. In some aspects, the antibody comprises a V_{H} sequence comprising, consisting of, or consisting essentially of SEQ ID NO: 2755. In some aspects, the antibody comprises a V_{H} sequence comprising, consisting of, or consisting essentially of SEQ ID NO:2756. In some aspects, the antibody comprises a V_{H} sequence comprising, consisting of, or consisting essentially of SEQ ID NO:2757. In some aspects, the antibody comprises a V_{H} sequence comprising, consisting of, or consisting essentially of SEQ ID NO:2758. In some aspects, the antibody comprises a V_{H} sequence comprising, consisting of, or consisting essentially of SEQ ID NO:2759. In some aspects, the antibody comprises a V_{H} sequence comprising, consisting of, or consisting essentially of SEQ ID NO: 2760. In some aspects, the antibody comprises a V_{H} sequence comprising, consisting of, or consisting essentially of SEQ ID NO:2761. In some aspects, the antibody comprises a V_{H} sequence comprising, consisting of, or consisting essentially of SEQ ID NO:2762. In some aspects, the antibody comprises a V_{H} sequence comprising, consisting of, or consisting essentially of SEQ ID NO:2763. In some aspects, the antibody comprises a V_{H} sequence comprising, consisting of, or consisting essentially of SEQ ID NO:2764. In some aspects, the antibody comprises a V_{H} sequence comprising, consisting of, or consisting essentially of SEQ ID NO: 2765. In some aspects, the antibody comprises a V_{H} sequence comprising, consisting of, or consisting essentially of SEQ ID NO:2766. In some aspects, the antibody comprises a V_{H} sequence comprising, consisting of, or consisting essentially of SEQ ID NO:2767. In some aspects, the antibody comprises a V_{H} sequence comprising, consisting of, or consisting essentially of SEQ ID NO:2768. In some aspects, the antibody comprises a V_{H} sequence comprising, consisting of, or consisting essentially of SEQ ID NO:2769. In some aspects, the antibody comprises a V_{H} sequence comprising, consisting of, or consisting essentially of SEQ ID NO: 2770. In some aspects, the antibody comprises a V_{H} sequence comprising, consisting of, or consisting essentially of SEQ ID NO:2771. In some aspects, the antibody comprises a V_{H} sequence comprising, consisting of, or consisting essentially of SEQ ID NO:2772. In some aspects, the antibody comprises a V_{H} sequence comprising, consisting of, or consisting essentially of SEQ ID NO:2773. In some aspects, the antibody comprises a V_{H} sequence comprising, consisting of, or consisting essentially of SEQ ID NO:2774. In some aspects, the antibody comprises a V_{H} sequence comprising, consisting of, or consisting essentially of SEQ ID NO: 2775. In some aspects, the antibody comprises a V_{H} sequence comprising, consisting of, or consisting essentially of SEQ ID NO:2776. In some aspects, the antibody comprises a V_{H} sequence comprising, consisting of, or consisting essentially of SEQ ID NO:2777. In some aspects, the antibody comprises a V_{H} sequence comprising, consisting of, or consisting essentially of SEQ ID NO:2778. In some aspects, the antibody comprises a V_{H} sequence comprising, consisting of, or consisting essentially of SEQ ID NO:2779. In some aspects, the antibody comprises a V_{H} sequence comprising, consisting of, or consisting essentially of SEQ ID NO: 2780. In some aspects, the antibody comprises a V_{H} sequence comprising, consisting of, or consisting essentially of SEQ ID NO:2781. In some aspects, the antibody comprises a V_{H} sequence comprising, consisting of, or consisting essentially of SEQ ID NO:2782. In some aspects, the antibody comprises a V_{H} sequence comprising, consisting of, or consisting essentially of SEQ ID NO:2783. In some aspects, the antibody comprises a V_{H} sequence comprising, consisting of, or consisting essentially of SEQ ID NO:2784. In some aspects, the antibody comprises a V_{H} sequence comprising, consisting of, or consisting essentially of SEQ ID NO: 2785. In some aspects, the antibody comprises a V_{H} sequence comprising, consisting of, or consisting essentially of SEQ ID NO:2786. In some aspects, the antibody comprises a V_{H} sequence comprising, consisting of, or consisting essentially of SEQ ID NO:2787. In some aspects, the antibody comprises a V_{H} sequence comprising, consisting of, or consisting essentially of SEQ ID NO:2788. In some aspects, the antibody comprises a V_{H} sequence comprising, consisting of, or consisting essentially of SEQ ID NO:2789. In some aspects, the antibody comprises a V_{H} sequence comprising, consisting of, or consisting essentially of SEQ ID NO: 2790. In some aspects, the antibody comprises a V_{H} sequence comprising, consisting of, or consisting essentially of SEQ ID NO:2791. In some aspects, the antibody comprises a V_{H} sequence comprising, consisting of, or consisting essentially of SEQ ID NO:2792. In some aspects, the antibody comprises a V_{H} sequence comprising, consisting of, or consisting essentially of SEQ ID NO:2793. In some aspects, the antibody comprises a V_{H} sequence comprising, consisting of, or consisting essentially of SEQ ID NO:2794. In some aspects, the antibody comprises a V_{H} sequence comprising, consisting of, or consisting essentially of SEQ ID NO: 2795. In some aspects, the antibody comprises a V_{H} sequence comprising, consisting of, or consisting essentially of SEQ ID NO:2796. In some aspects, the antibody comprises a V_{H} sequence comprising, consisting of, or consisting essentially of SEQ ID NO:2797. In some aspects, the antibody comprises a V_{H} sequence comprising, consisting of, or consisting essentially of SEQ ID NO:2798. In some aspects, the antibody comprises a V_{H} sequence comprising, consisting of, or consisting essentially of SEQ ID NO:2799. In some aspects, the antibody comprises a V_{H} sequence comprising, consisting of, or consisting essentially of SEQ ID NO: 2800. In some aspects, the antibody comprises a V_{H} sequence comprising, consisting of, or consisting essentially of SEQ ID NO:2801. In some aspects, the antibody comprises a V_{H} sequence comprising, consisting of, or consisting essentially of SEQ ID NO:2802. In some aspects, the antibody comprises a V_{H} sequence comprising, consisting of, or consisting essentially of SEQ ID NO:2803. In some aspects, the antibody comprises a V_{H} sequence comprising, consisting of, or consisting essentially of SEQ ID NO:2804. In some aspects, the antibody comprises a V_{H} sequence comprising, consisting of, or consisting essentially of SEQ ID NO: 2805. In some aspects, the antibody comprises a V_{H} sequence comprising, consisting of, or consisting essentially of SEQ ID NO:2806. In some aspects, the antibody comprises a V_{H} sequence comprising, consisting of, or consisting essentially of SEQ ID NO:2807. In some aspects, the antibody comprises a V_{H} sequence comprising, consisting of, or consisting essentially of SEQ ID NO:2808. In some aspects, the antibody comprises a V_{H} sequence comprising, consisting of, or consisting essentially of SEQ ID NO:2809. In some aspects, the antibody comprises a V_{H} sequence comprising, consisting of, or consisting essentially of SEQ ID NO: 2810. In some aspects, the antibody comprises a V_{H} sequence comprising, consisting of, or consisting essentially of SEQ ID NO:2811. In some aspects, the antibody comprises a V_{H} sequence comprising, consisting of, or consisting essentially of SEQ ID NO:2812. In some aspects, the antibody comprises a V_{H} sequence comprising, consisting of, or consisting essentially of SEQ ID NO:2813. In some aspects, the antibody comprises a V_{H} sequence comprising, consisting of, or consisting essentially of SEQ ID NO:2814. In some aspects, the antibody comprises a V_{H} sequence comprising, consisting of, or consisting essentially of SEQ ID NO: 2815. In some aspects, the antibody comprises a V_{H} sequence comprising, consisting of, or consisting essentially of SEQ ID NO:2816. In some aspects, the antibody comprises a V_{H} sequence comprising, consisting of, or consisting essentially of SEQ ID NO:2817. In some aspects, the antibody comprises a V_{H} sequence comprising, consisting of, or consisting essentially of SEQ ID NO:2818. In some aspects, the antibody comprises a V_{H} sequence comprising, consisting of, or consisting essentially of SEQ ID NO:2819. In some aspects, the antibody comprises a V_{H} sequence comprising, consisting of, or consisting essentially of SEQ ID NO: 2820. In some aspects, the antibody comprises a V_{H} sequence comprising, consisting of, or consisting essentially of SEQ ID NO:2821. In some aspects, the antibody comprises a V_{H} sequence comprising, consisting of, or consisting essentially of SEQ ID NO:2822. In some aspects, the antibody comprises a V_{H} sequence comprising, consisting of, or consisting essentially of SEQ ID NO:2823. In some aspects, the antibody comprises a V_{H} sequence comprising, consisting of, or consisting essentially of SEQ ID NO:2824.

In some aspects, the antibody comprises a V_{H} sequence comprising, consisting of, or consisting essentially of SEQ ID NO:2825. In some aspects, the antibody comprises a V_{H} sequence comprising, consisting of, or consisting essentially of SEQ ID NO:2826. In some aspects, the antibody comprises a V_{H} sequence comprising, consisting of, or consisting essentially of SEQ ID NO:2827. In some aspects, the antibody comprises a V_{H} sequence comprising, consisting of, or consisting essentially of SEQ ID NO:2828. In some aspects, the antibody comprises a V_{H} sequence comprising, consisting of, or consisting essentially of SEQ ID NO: 2829. In some aspects, the antibody comprises a V_{H} sequence comprising, consisting of, or consisting essentially of SEQ ID NO:2830. In some aspects, the antibody comprises a V_{H} sequence comprising, consisting of, or consisting essentially of SEQ ID NO:2831. In some aspects, the antibody comprises a V_{H} sequence comprising, consisting of, or consisting essentially of SEQ ID NO:2832. In some aspects, the antibody comprises a V_{H} sequence comprising, consisting of, or consisting essentially of SEQ ID NO:2833. In some aspects, the antibody comprises a V_{H} sequence comprising, consisting of, or consisting essentially of SEQ ID NO: 2834. In some aspects, the antibody comprises a V_{H} sequence comprising, consisting of, or consisting essentially of SEQ ID NO:2835. In some aspects, the antibody comprises a V_{H} sequence comprising, consisting of, or consisting essentially of SEQ ID NO:2836. In some aspects, the antibody comprises a V_{H} sequence comprising, consisting of, or consisting essentially of SEQ ID NO:2837. In some aspects, the antibody comprises a V_{H} sequence comprising, consisting of, or consisting essentially of SEQ ID NO:2838.

In some aspects, the antibody comprises a V_{H} sequence comprising, consisting of, or consisting essentially of SEQ ID NO:2840. In some aspects, the antibody comprises a V_{H} sequence comprising, consisting of, or consisting essentially of SEQ ID NO:2846. In some aspects, the antibody comprises a V_{H} sequence comprising, consisting of, or consisting essentially of SEQ ID NO:2851. In some aspects, the antibody comprises a V_{H} sequence comprising, consisting of, or consisting essentially of SEQ ID NO:2857. In some aspects, the antibody comprises a V_{H} sequence comprising, consisting of, or consisting essentially of SEQ ID NO: 2859. In some aspects, the antibody comprises a V_{H} sequence comprising, consisting of, or consisting essentially of SEQ ID NO:2860.

### 2.3.1. Variants of V_{H} Sequences

In some embodiments, the V_{H} sequences provided herein comprise, consist of, or consist essentially of a variant of an illustrative V_{H} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 2.4 V_{L} Sequences Comprising Illustrative CDRs

In some embodiments, the antibody comprises a V_{L} sequence comprising one or more CDR-L sequences comprising, consisting of, or consisting essentially of one or more illustrative CDR-L sequences provided in this disclosure, and variants thereof.

### 2.4.1. V_{L} Sequences Comprising Illustrative Kabat CDRs

In some embodiments, the antibody comprises a V_{L} sequence comprising one or more Kabat CDR-L sequences comprising, consisting of, or consisting essentially of one or more illustrative Kabat CDR-L sequences provided in this disclosure, and variants thereof.

### 2.4.1.1. Kabat CDR-L1+Kabat CDR-L2+Kabat CDR-L3

In some embodiments, the antibody comprises a V_{L} sequence comprising a Kabat CDR-L1 sequence comprising, consisting of, or consisting essentially of a sequence selected from SEQ ID NOs: 2066, 2072, 2077, 2083, 2085, 2086, and 2176, a Kabat CDR-L2 sequence comprising, consisting of, or consisting essentially of a sequence selected from SEQ ID NOs: 2290, 2296, 2301, 2307, 2309, 2310, and 2400, and a Kabat CDR-L3 sequence comprising, consisting of, or consisting essentially of a sequence selected from SEQ ID NOs: 2514, 2520, 2525, 2531, 2533, 2534, and 2624. In some aspects, the Kabat CDR-L1 sequence, Kabat CDR-L2 sequence, and Kabat CDR-L3 sequence are all from a single illustrative V_{L} sequence provided in this disclosure. For example, in some aspects, the Kabat CDR-L1, Kabat CDR-L2, and Kabat CDR-L3 are all from a single illustrative V_{L} sequence selected from SEQ ID NOs: 2951, 2957, 2962, 2968, 2970, 2971, and 3061.

### 2.4.1.2. Variants of V_{L} Sequences Comprising Illustrative Kabat CDRs

In some embodiments, the V_{L} sequences provided herein comprise a variant of an illustrative Kabat CDR-L3, CDR-L2, and/or CDR-L1 sequence provided in this disclosure.

In some aspects, the Kabat CDR-L3 sequence comprises, consists of, or consists essentially of a variant of an illustrative Kabat CDR-L3 sequence provided in this disclosure. In some aspects, the Kabat CDR-L3 sequence comprises, consists of, or consists essentially of a sequence having at least 70%, 75%, 80%, 85%, 90%, 95%, 96%, 97%, 98%, or 99% identity with any of the illustrative Kabat CDR-L3 sequences provided in this disclosure. In some aspects, the Kabat CDR-L3 sequence comprises, consists of, or consists essentially of any of the illustrative Kabat CDR-L3 sequences provided in this disclosure, with 1, 2, or 3 amino acid substitutions. In some aspects, the amino acid substitutions are conservative amino acid substitutions.

In some aspects, the Kabat CDR-L2 sequence comprises, consists of, or consists essentially of a variant of an illustrative Kabat CDR-L2 sequence provided in this disclosure. In some aspects, the Kabat CDR-L2 sequence comprises, consists of, or consists essentially of a sequence having at least 70%, 75%, 80%, 85%, 90%, 95%, 96%, 97%, 98%, or 99% identity with any of the illustrative Kabat CDR-L2 sequences provided in this disclosure. In some aspects, the Kabat CDR-L2 sequence comprises, consists of, or consists essentially of any of the illustrative Kabat CDR-L2 sequences provided in this disclosure, with 1, 2, or 3 amino acid substitutions. In some aspects, the amino acid substitutions are conservative amino acid substitutions.

In some aspects, the Kabat CDR-L1 sequence comprises, consists of, or consists essentially of a variant of an illustrative Kabat CDR-L1 sequence provided in this disclosure. In some aspects, the Kabat CDR-L1 sequence comprises, consists of, or consists essentially of a sequence having at least 70%, 75%, 80%, 85%, 90%, 95%, 96%, 97%, 98%, or 99% identity with any of the illustrative Kabat CDR-L1 sequences provided in this disclosure. In some aspects, the Kabat CDR-L1 sequence comprises, consists of, or consists essentially of any of the illustrative Kabat CDR-L1 sequences provided in this disclosure, with 1, 2, or 3 amino acid substitutions. In some aspects, the amino acid substitutions are conservative amino acid substitutions.

### 2.4.2. V_{L} Sequences Comprising Illustrative Chothia CDRs

In some embodiments, the antibody comprises a V_{L} sequence comprising one or more Chothia CDR-L sequences comprising, consisting of, or consisting essentially of one or more illustrative Chothia CDR-L sequences provided in this disclosure, and variants thereof.

### 2.4.2.1. Chothia CDR-L1+Chothia CDR-L2+Chothia CDR-L3

In some embodiments, the antibody comprises a V_{L} sequence comprising a Chothia CDR-L1 sequence comprising, consisting of, or consisting essentially of a sequence selected from SEQ ID NOs: 1954, 1960, 1965, 1971, 1973, 1974 and 2064, a Chothia CDR-L2 sequence comprising, consisting of, or consisting essentially of a sequence selected from SEQ ID NOs: 2178, 2184, 2189, 2195, 2197, 2198, and 2288, and a Chothia CDR-L3 sequence comprising, consisting of, or consisting essentially of a sequence selected from SEQ ID NOs: 2402, 2408, 2413, 2419, 2421, 2422, and 2512. In some aspects, the Chothia CDR-L1 sequence, Chothia CDR-L2 sequence, and Chothia CDR-L3 sequence are all from a single illustrative V_{L} sequence provided in this disclosure. For example, in some aspects, the Chothia CDR-L1, Chothia CDR-L2, and Chothia CDR-L3 are all from a single illustrative V_{L} sequence selected from SEQ ID NOs: 2951, 2957, 2962, 2968, 2970, 2971, and 3061.

### 2.4.2.2. Variants of V_{L} Sequences Comprising Illustrative Chothia CDRs

In some embodiments, the V_{H} sequences provided herein comprise a variant of an illustrative Chothia CDR-L3, CDR-L2, and/or CDR-L1 sequence provided in this disclosure.

In some aspects, the Chothia CDR-L3 sequence comprises, consists of, or consists essentially of a variant of an illustrative Chothia CDR-L3 sequence provided in this disclosure. In some aspects, the Chothia CDR-L3 sequence comprises, consists of, or consists essentially of a sequence having at least 70%, 75%, 80%, 85%, 90%, 95%, 96%, 97%, 98%, or 99% identity with any of the illustrative Chothia CDR-L3 sequences provided in this disclosure. In some aspects, the Chothia CDR-L3 sequence comprises, consists of, or consists essentially of any of the illustrative Chothia CDR-L3 sequences provided in this disclosure, with 1, 2, or 3 amino acid substitutions. In some aspects, the amino acid substitutions are conservative amino acid substitutions.

In some aspects, the Chothia CDR-L2 sequence comprises, consists of, or consists essentially of a variant of an illustrative Chothia CDR-L2 sequence provided in this disclosure. In some aspects, the Chothia CDR-L2 sequence comprises, consists of, or consists essentially of a sequence having at least 70%, 75%, 80%, 85%, 90%, 95%, 96%, 97%, 98%, or 99% identity with any of the illustrative Chothia CDR-L2 sequences provided in this disclosure. In some aspects, the Chothia CDR-L2 sequence comprises, consists of, or consists essentially of any of the illustrative Chothia CDR-L2 sequences provided in this disclosure, with 1, 2, or 3 amino acid substitutions. In some aspects, the amino acid substitutions are conservative amino acid substitutions.

In some aspects, the Chothia CDR-L1 sequence comprises, consists of, or consists essentially of a variant of an illustrative Chothia CDR-L1 sequence provided in this disclosure. In some aspects, the Chothia CDR-L1 sequence comprises, consists of, or consists essentially of a sequence having at least 70%, 75%, 80%, 85%, 90%, 95%, 96%, 97%, 98%, or 99% identity with any of the illustrative Chothia CDR-L1 sequences provided in this disclosure. In some aspects, the Chothia CDR-L1 sequence comprises, consists of, or consists essentially of any of the illustrative Chothia CDR-L1 sequences provided in this disclosure, with 1, 2, or 3 amino acid substitutions. In some aspects, the amino acid substitutions are conservative amino acid substitutions.

### 2.5. V_{L} Sequences

In some embodiments, the antibody comprises, consists of, or consists essentially of a V_{L} sequence of an scFv sequence provided in SEQ ID NOs.: 3073 and 3079. In some embodiments, the antibody comprises, consists of, or consists essentially of a V_{L} sequence provided in SEQ ID NOs.: 2951, 2957, 2962, 2968, 2970, 2971, and 3061.

In some embodiments, the antibody comprises a V_{L} sequence comprising, consisting of, or consisting essentially of a sequence selected from SEQ ID NOs: 2951, 2957, 2962, 2968, 2970, 2971, and 3061.

In some aspects, the antibody comprises a V_{L} sequence comprising, consisting of, or consisting essentially of SEQ ID NO:2951. In some aspects, the antibody comprises a V_{L} sequence comprising, consisting of, or consisting essentially of SEQ ID NO:2957. In some aspects, the antibody comprises a V_{L} sequence comprising, consisting of, or consisting essentially of SEQ ID NO:2962. In some aspects, the antibody comprises a V_{L} sequence comprising, consisting of, or consisting essentially of SEQ ID NO:2968. In some aspects, the antibody comprises a V_{L} sequence comprising, consisting of, or consisting essentially of SEQ ID NO: 2970. In some aspects, the antibody comprises a V_{L} sequence comprising, consisting of, or consisting essentially of SEQ ID NO:2971. In some aspects, the antibody comprises a V_{L} sequence comprising, consisting of, or consisting essentially of SEQ ID NO:3061.

### 2.5.1. Variants of V_{L} Sequences

In some embodiments, the V_{L} sequences provided herein comprise, consist of, or consist essentially of a variant of an illustrative V_{L} sequence provided in this disclosure.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 2.6. Pairs

2.6.1. V_{H}-V_{L} Pairs

In some embodiments, the antibody comprises a V_{H} sequence and a V_{L} sequence.

In some aspects, the V_{H} sequence is a V_{H} sequence comprising, consisting of, or consisting essentially of SEQ ID NOs: 2727, 2736, 2739, 2751-2838, 2840, 2846, 2851, 2857, 2859, and 2860, and the V_{L} sequence is a V_{L} sequence comprising, consisting of, or consisting essentially of SEQ ID NOs: 2951, 2957, 2962, 2968, 2970, 2971, and 3061.

In some embodiments, the antibody comprises a V_{H}/V_{L} pair together comprising, consisting of, or consisting essentially of the sequences of any pair selected from the group consisting of SEQ ID NOs: 2727/3061, 2736/3061, 2739/3061, 2751/3061, 2752/3061, 2753/3061, 2754/3061, 2755/3061, 2756/3061, 2757/3061, 2758/3061, 2759/3061, 2760/3061, 2761/3061, 2762/3061, 2763/3061, 2764/3061, 2765/3061, 2766/3061, 2767/3061, 2768/3061, 2769/3061, 2770/3061, 2771/3061, 2772/3061, 2773/3061, 2774/3061, 2775/3061, 2776/3061, 2777/3061, 2778/3061, 2779/3061, 2780/3061, 2781/3061, 2782/3061, 2783/3061, 2784/3061, 2785/3061, 2786/3061, 2787/3061, 2788/3061, 2789/3061, 2790/3061, 2791/3061, 2792/3061, 2793/3061, 2794/3061, 2795/3061, 2796/3061, 2797/3061, 2798/3061, 2799/3061, 2800/3061, 2801/3061, 2802/3061, 2803/3061, 2804/3061, 2805/3061, 2806/3061, 2807/3061, 2808/3061, 2809/3061, 2810/3061, 2811/3061, 2812/3061, 2813/3061, 2814/3061, 2815/3061, 2816/3061, 2817/3061, 2818/3061, 2819/3061, 2820/3061, 2821/3061, 2822/3061, 2823/3061, 2824/3061, 2825/3061, 2826/3061, 2827/3061, 2828/3061, 2829/3061, 2830/3061, 2831/3061, 2832/3061, 2833/3061, 2834/3061, 2835/3061, 2836/3061, 2837/3061, 2838/3061, 2840/2951, 2846/2957, 2851/2962, 2857/2968, 2859/2970, and 2860/2971.

In some aspects, the antibody comprises a V_{H}/V_{L} pair together comprising, consisting of, or consisting essentially of the sequences of any pair as shown in the Table 5 below:

TABLE 5
V_{H}/V_{L} pairs.

| | V_{H} Sequence | VL Sequence |
|---|---|---|
| Antibody | (SEQ ID NO) | (SEQ ID NO) |
| SRP2799-A05 | 2727 | 3061 |
| SRP2799-B03 | 2736 | 3061 |
| SRP2799-B06 | 2739 | 3061 |
| SRP2900-A01 | 2751 | 3061 |
| SRP2900-A02 | 2752 | 3061 |
| SRP2900-A03 | 2753 | 3061 |
| SRP2900-A04 | 2754 | 3061 |
| SRP2900-A05 | 2755 | 3061 |
| SRP2900-A06 | 2756 | 3061 |
| SRP2900-A07 | 2757 | 3061 |
| SRP2900-A08 | 2758 | 3061 |
| SRP2900-A09 | 2759 | 3061 |
| SRP2900-A10 | 2760 | 3061 |
| SRP2900-A11 | 2761 | 3061 |
| SRP2900-B01 | 2762 | 3061 |
| SRP2900-B02 | 2763 | 3061 |
| SRP2900-B03 | 2764 | 3061 |
| SRP2900-B04 | 2765 | 3061 |
| SRP2900-B05 | 2766 | 3061 |
| SRP2900-B06 | 2767 | 3061 |
| SRP2900-B07 | 2768 | 3061 |
| SRP2900-B08 | 2769 | 3061 |
| SRP2900-B09 | 2770 | 3061 |
| SRP2900-B10 | 2771 | 3061 |
| SRP2900-B11 | 2772 | 3061 |
| SRP2900-C01 | 2773 | 3061 |
| SRP2900-C02 | 2774 | 3061 |
| SRP2900-C03 | 2775 | 3061 |
| SRP2900-C04 | 2776 | 3061 |
| SRP2900-C05 | 2777 | 3061 |
| SRP2900-C06 | 2778 | 3061 |
| SRP2900-C07 | 2779 | 3061 |
| SRP2900-C08 | 2780 | 3061 |
| SRP2900-C09 | 2781 | 3061 |
| SRP2900-C10 | 2782 | 3061 |
| SRP2900-C11 | 2783 | 3061 |
| SRP2900-D01 | 2784 | 3061 |
| SRP2900-D02 | 2785 | 3061 |
| SRP2900-D03 | 2786 | 3061 |
| SRP2900-D04 | 2787 | 3061 |
| SRP2900-D05 | 2788 | 3061 |
| SRP2900-D06 | 2789 | 3061 |
| SRP2900-D07 | 2790 | 3061 |
| SRP2900-D08 | 2791 | 3061 |
| SRP2900-D09 | 2792 | 3061 |
| SRP2900-D10 | 2793 | 3061 |
| SRP2900-D11 | 2794 | 3061 |
| SRP2900-E01 | 2795 | 3061 |
| SRP2900-E02 | 2796 | 3061 |
| SRP2900-E03 | 2797 | 3061 |
| SRP2900-E04 | 2798 | 3061 |
| SRP2900-E05 | 2799 | 3061 |
| SRP2900-E06 | 2800 | 3061 |
| SRP2900-E07 | 2801 | 3061 |
| SRP2900-E08 | 2802 | 3061 |
| SRP2900-E09 | 2803 | 3061 |
| SRP2900-E10 | 2804 | 3061 |
| SRP2900-E11 | 2805 | 3061 |
| SRP2900-F01 | 2806 | 3061 |
| SRP2900-F02 | 2807 | 3061 |
| SRP2900-F03 | 2808 | 3061 |
| SRP2900-F04 | 2809 | 3061 |
| SRP2900-F05 | 2810 | 3061 |
| SRP2900-F06 | 2811 | 3061 |
| SRP2900-F07 | 2812 | 3061 |
| SRP2900-F08 | 2813 | 3061 |
| SRP2900-F09 | 2814 | 3061 |
| SRP2900-F10 | 2815 | 3061 |
| SRP2900-F11 | 2816 | 3061 |
| SRP2900-G01 | 2817 | 3061 |
| SRP2900-G02 | 2818 | 3061 |
| SRP2900-G03 | 2819 | 3061 |
| SRP2900-G04 | 2820 | 3061 |
| SRP2900-G05 | 2821 | 3061 |
| SRP2900-G06 | 2822 | 3061 |
| SRP2900-G07 | 2823 | 3061 |
| SRP2900-G08 | 2824 | 3061 |
| SRP2900-G09 | 2825 | 3061 |
| SRP2900-G10 | 2826 | 3061 |
| SRP2900-G11 | 2827 | 3061 |
| SRP2900-H01 | 2828 | 3061 |
| SRP2900-H02 | 2829 | 3061 |
| SRP2900-H03 | 2830 | 3061 |
| SRP2900-H04 | 2831 | 3061 |
| SRP2900-H05 | 2832 | 3061 |
| SRP2900-H06 | 2833 | 3061 |
| SRP2900-H07 | 2834 | 3061 |
| SRP2900-H08 | 2835 | 3061 |
| SRP2900-H09 | 2836 | 3061 |
| SRP2900-H10 | 2837 | 3061 |
| SRP2900-H11 | 2838 | 3061 |
| SRP2842-B01 | 2840 | 2951 |
| SRP2842-G04 | 2846 | 2957 |
| SRP2901-B05 | 2851 | 2962 |
| SRP2901-D03 | 2857 | 2968 |
| SRP2901-E03 | 2859 | 2970 |
| SRP2901-F01 | 2860 | 2971 |

2.6.1.1. Variants of V_{H}-V_{L} Pairs

In some embodiments, the V_{H}-V_{L} pairs provided herein comprise a variant of an illustrative V_{H} and/or V_{L} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 2.7. Antibodies Comprising all Six CDRs

In some embodiments, the antibody comprises a CDR-H1 sequence, a CDR-H2 sequence, a CDR-H3 sequence, a CDR-L1 sequence, and a CDR-L3 sequence. In some aspects, the CDR sequences are part of a V_{H} (for CDR-H) or V_{L} (for CDR-L).

In some aspects, the CDR-H1 sequence is a Chothia CDR-H1 sequence comprising, consisting of, or consisting essentially of SEQ ID NOs: 105, 114, 117, 129-216, 218, 224, 229, 235, 237, and 238; the CDR-H2 sequence is a Chothia CDR-H2 sequence comprising, consisting of, or consisting essentially of SEQ ID NOs: 755, 764, 767, 779-866, 868, 874, 879, 885, 887, and 888; the CDR-H3 sequence is a Chothia CDR-H3 sequence comprising, consisting of, or consisting essentially of SEQ ID NOs: 1405, 1414, 1417, 1429-1516, 1518, 1524, 1529, 1535, 1537, and 1538; the CDR-L1 sequence is a Chothia CDR-L1 sequence comprising, consisting of, or consisting essentially of SEQ ID NOs: 1954, 1960, 1965, 1971, 1973, 1974 and 2064; the CDR-L2 sequence is a Chothia CDR-L2 sequence comprising, consisting of, or consisting essentially of SEQ ID NOs: 2178, 2184, 2189, 2195, 2197, 2198, and 2288; and the CDR-L3 sequence is a Chothia CDR-L3 sequence comprising, consisting of, or consisting essentially of SEQ ID NOs: 2402, 2408, 2413, 2419, 2421, 2422, and 2512.

In some aspects, the CDR-H1 sequence is a Kabat CDR-H1 sequence comprising, consisting of, or consisting essentially of SEQ ID NOs: 430, 439, 442, 454-541, 543, 549, 554, 560, 562, and 563; the CDR-H2 sequence is a Kabat CDR-H2 sequence comprising, consisting of, or consisting essentially of SEQ ID NOs: 1080, 1089, 1092, 1104-1191, 1193, 1199, 1204, 1200, 1212, and 1213; the CDR-H3 sequence is a Kabat CDR-H3 sequence comprising, consisting of, or consisting essentially of SEQ ID NOs: 1730, 1739, 1742, 1754-1841, 1843, 1849, 1854, 1860, 1862, and 1863; the CDR-L1 sequence is a Kabat CDR-L1 sequence comprising, consisting of, or consisting essentially of SEQ ID NOs: 2066, 2072, 2077, 2083, 2085, 2086, and 2176; the CDR-L2 sequence is a Kabat CDR-L2 sequence comprising, consisting of, or consisting essentially of SEQ ID NOs: 2290, 2296, 2301, 2307, 2309, 2310, and 2400; and the CDR-L3 sequence is a Kabat CDR-L3 sequence comprising, consisting of, or consisting essentially of SEQ ID NOs: 2514, 2520, 2525, 2531, 2533, 2534, and 2624.

In some aspects, the antibody comprises three heavy chain CDRs from a V_{H} sequence selected from SEQ ID NOs: 2727, 2736, 2739, 2751-2838, 2840, 2846, 2851, 2857, 2859, and 2860, or variants thereof, and three light chain CDRs from a V_{L} sequence selected from SEQ ID NOs: 2951, 2957, 2962, 2968, 2970, 2971, and 3061, or variants thereof. The CDRs can be according to any CDR scheme known to the person of skill. In certain embodiments, the CDRs are Kabat CDRs. In certain embodiments, the CDRs are Chothia CDRs.

In some embodiments, the antibody comprises three heavy chain CDRs, or variants thereof, and three light chain CDRs, or variants thereof, from a V_{H}/V_{L} pair selected from the group consisting of the group consisting of SEQ ID NOs: 2727/3061, 2736/3061, 2739/3061, 2751/3061, 2752/3061, 2753/3061, 2754/3061, 2755/3061, 2756/3061, 2757/3061, 2758/3061, 2759/3061, 2760/3061, 2761/3061, 2762/3061, 2763/3061, 2764/3061, 2765/3061, 2766/3061, 2767/3061, 2768/3061, 2769/3061, 2770/3061, 2771/3061, 2772/3061, 2773/3061, 2774/3061, 2775/3061, 2776/3061, 2777/3061, 2778/3061, 2779/3061, 2780/3061, 2781/3061, 2782/3061, 2783/3061, 2784/3061, 2785/3061, 2786/3061, 2787/3061, 2788/3061, 2789/3061, 2790/3061, 2791/3061, 2792/3061, 2793/3061, 2794/3061, 2795/3061, 2796/3061, 2797/3061, 2798/3061, 2799/3061, 2800/3061, 2801/3061, 2802/3061, 2803/3061, 2804/3061, 2805/3061, 2806/3061, 2807/3061, 2808/3061, 2809/3061, 2810/3061, 2811/3061, 2812/3061, 2813/3061, 2814/3061, 2815/3061, 2816/3061, 2817/3061, 2818/3061, 2819/3061, 2820/3061, 2821/3061, 2822/3061, 2823/3061, 2824/3061, 2825/3061, 2826/3061, 2827/3061, 2828/3061, 2829/3061, 2830/3061, 2831/3061, 2832/3061, 2833/3061, 2834/3061, 2835/3061, 2836/3061, 2837/3061, 2838/3061, 2840/2951, 2846/2957, 2851/2962, 2857/2968, 2859/2970, and 2860/2971.

In some embodiments, the antibody comprises three heavy chain CDRs, or variants thereof, and three light chain CDRs, or variants thereof, from a V_{H}/V_{L} pair selected from the group consisting of the group consisting of SEQ ID NOs: 2727/3061, 2736/3061, and 2739/3061. In some embodiments, the antibody comprises three heavy chain CDRs, or variants thereof, and three light chain CDRs, or variants thereof, from the V_{H}/V_{L} pair of SEQ ID NOS: 2752/3061.

The CDRs can be according to any CDR scheme known to the person of skill. In certain embodiments, the CDRs are Kabat CDRs. In certain embodiments, the CDRs are Chothia CDRs. In certain embodiments, the antibody further comprises the framework regions of the V_{H}/V_{L} pair.

### 2.7.1. Variants of Antibodies Comprising all Six CDRs

In some embodiments, the CDR-H1, CDR-H2, CDR-H3, CDR-L1, CDR-L2, and CDR-L3 provided herein comprise a variant of an illustrative CDR-H1, CDR-H2, CDR-H3, CDR-L1, CDR-L2, and/or CDR-L3 sequence provided in this disclosure.

In some aspects, the CDR-H1 sequence comprises, consists of, or consists essentially of a variant of an illustrative Chothia or Kabat CDR-H1 sequence provided in this disclosure. In some aspects, the CDR-H1 sequence comprises, consists of, or consists essentially of a sequence having at least 70%, 75%, 80%, 85%, 90%, 95%, 96%, 97%, 98%, or 99% identity with any of the illustrative Chothia or Kabat CDR-H1 sequences provided in this disclosure. In some aspects, the CDR-H1 sequence comprises, consists of, or consists essentially of any of the illustrative Chothia or Kabat CDR-H1 sequences provided in this disclosure, with 1, 2, or 3 amino acid substitutions. In some aspects, the amino acid substitutions are conservative amino acid substitutions.

In some aspects, the CDR-H2 sequence comprises, consists of, or consists essentially of a variant of an illustrative Chothia or Kabat CDR-H2 sequence provided in this disclosure. In some aspects, the CDR-H2 sequence comprises, consists of, or consists essentially of a sequence having at least 70%, 75%, 80%, 85%, 90%, 95%, 96%, 97%, 98%, or 99% identity with any of the illustrative Chothia or Kabat CDR-H2 sequences provided in this disclosure. In some aspects, the CDR-H2 sequence comprises, consists of, or consists essentially of any of the illustrative Chothia or Kabat CDR-H2 sequences provided in this disclosure, with 1, 2, or 3 amino acid substitutions. In some aspects, the amino acid substitutions are conservative amino acid substitutions.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, 95%, 96%, 97%, 98%, or 99% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, 95%, 96%, 97%, 98%, or 99% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, 95%, 96%, 97%, 98%, or 99% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, 95%, 96%, 97%, 98%, or 99% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 3. Conjugates

Provided herein are conjugates of antibodies to TF. The conjugates comprise an antibody to TF covalently linked directly or indirectly, via a linker, to a payload. In some embodiments, the conjugate comprises an antibody that specifically binds to TF linked site-specifically to at least one payload moiety, and the antibody comprises one or more non-natural amino acids. In certain embodiments, the antibody is linked to one payload. In further embodiments, the antibody is linked to more than one payload. In certain embodiments, the antibody is linked to two, three, four, five, six, seven, eight, nine, ten, or more payloads.

The payload can be any payload deemed useful by the practitioner of skill. In certain embodiments, the payload is a therapeutic moiety. In certain embodiments, the payload is a diagnostic moiety, e.g., a label. Useful payloads are described in the sections and examples below.

The linker can be any linker capable of forming at least one bond to the antibody and at least one bond to a payload. Useful linkers are described the sections and examples below.

In the conjugates provided herein, the antibody can be any antibody with binding specificity for TF. The TF can be from any species. In certain embodiments, the TF is a vertebrate TF. In certain embodiments, the TF is a mammalian TF. In certain embodiments, the TF is human TF. In certain embodiments, the TF is mouse TF. In certain embodiments, the TF is cynomolgus TF.

In certain embodiments, the antibody to TF competes with an antibody described herein for binding. In certain embodiments, the antibody to TF binds to the same epitope as an antibody described herein.

The antibody is typically a protein comprising multiple polypeptide chains. In certain embodiments, the antibody is a heterotetramer comprising two identical light (L) chains and two identical heavy (H) chains. Each light chain can be linked to a heavy chain by one covalent disulfide bond. Each heavy chain can be linked to the other heavy chain by one or more covalent disulfide bonds. Each heavy chain and each light chain can also have one or more intrachain disulfide bonds. As is known to those of skill in the art, each heavy chain typically comprises a variable domain (V_{H}) followed by a number of constant domains. Each light chain typically comprises a variable domain at one end (V_{L}) and a constant domain. As is known to those of skill in the art, antibodies typically have selective affinity for their target molecules, i.e., antigens.

The antibodies provided herein can have any antibody form known to those of skill in the art. They can be full-length, or fragments. Exemplary full-length antibodies include IgA, IgA1, IgA2, IgD, IgE, IgG, IgG1, IgG2, IgG3, IgG4, IgM, etc. Exemplary fragments include Fv, Fab, Fc, scFv, scFv-Fc, and etc.

In certain embodiments, the antibody of the conjugate comprises one, two, three, four, five, or six of the CDR sequences described herein. In certain embodiments, the antibody of the conjugate comprises a heavy chain variable domain (V_{H}) described herein. In certain embodiments, the antibody of the conjugate comprises a light chain variable domain (V_{L}) described herein. In certain embodiments, the antibody of the conjugate comprises a heavy chain variable domain (V_{H}) described herein and a light chain variable domain (V_{L}) described herein. In certain embodiments, the antibody of the conjugate comprises a paired heavy chain variable domain and a light chain variable domain described herein (V_{H}-V_{L} pair). In certain embodiments, the antibody of the conjugate comprises a heavy chain (HC) described herein. In certain embodiments, the antibody of the conjugate comprises a light chain (LC) described herein. In certain embodiments, the antibody of the conjugate comprises a heavy chain (HC) described herein and a light chain (LC) described herein. In certain embodiments, the antibody of the conjugate comprises a paired heavy chain and a light chain described herein (HC-LC pair). In certain embodiments, the antibody of the conjugate comprises a heavy chain (HC) described herein. In certain embodiments, the antibody of the conjugate comprises a light chain (LC) described herein. In certain embodiments, the antibody of the conjugate comprises a heavy chain (HC) described herein and a light chain (LC) described herein. In certain embodiments, the antibody of the conjugate comprises a paired heavy chain and a light chain described herein (HC-LC pair).

In certain embodiments, the antibody of the conjugate comprises any of the amino acid sequences of the antibodies described above. In certain embodiments, the antibody comprises any of the amino acid sequences above with up to 10 amino acid substitutions. In certain embodiments, the antibody comprises any of the amino acid sequences above with up to 9 amino acid substitutions. In certain embodiments, the antibody comprises any of the amino acid sequences above with up to 8 amino acid substitutions. In certain embodiments, the antibody comprises any of the amino acid sequences above with up to 7 amino acid substitutions. In certain embodiments, the antibody comprises any of the amino acid sequences above with up to 6 amino acid substitutions. In certain embodiments, the antibody comprises any of the amino acid sequences above with up to 5 amino acid substitutions. In certain embodiments, the antibody comprises any of the amino acid sequences above with up to 4 amino acid substitutions. In certain embodiments, the antibody comprises any of the amino acid sequences above with up to 3 amino acid substitutions. In certain embodiments, the antibody comprises any of the amino acid sequences above with up to 2 amino acid substitutions. In certain embodiments, the antibody comprises any of the amino acid sequences above with up to 1 conservative amino acid substitution. In some embodiments, the amino acid substitutions are conservative amino acid substitutions. For example, in certain embodiments, the antibody comprises any of the amino acid sequences above with up to 10 conservative amino acid substitutions. In certain embodiments, the antibody comprises any of the amino acid sequences above with up to 9 conservative amino acid substitutions. In certain embodiments, the antibody comprises any of the amino acid sequences above with up to 8 conservative amino acid substitutions. In certain embodiments, the antibody comprises any of the amino acid sequences above with up to 7 conservative amino acid substitutions. In certain embodiments, the antibody comprises any of the amino acid sequences above with up to 6 conservative amino acid substitutions. In certain embodiments, the antibody comprises any of the amino acid sequences above with up to 5 conservative amino acid substitutions. In certain embodiments, the antibody comprises any of the amino acid sequences above with up to 4 conservative amino acid substitutions. In certain embodiments, the antibody comprises any of the amino acid sequences above with up to 3 conservative amino acid substitutions. In certain embodiments, the antibody comprises any of the amino acid sequences above with up to 2 conservative amino acid substitutions. In certain embodiments, the antibody comprises any of the amino acid sequences above with up to 1 conservative amino acid substitution.

In certain embodiments, the antibody conjugate can be formed from an antibody that comprises one or more reactive groups. In certain embodiments, the antibody conjugate can be formed from an antibody comprising all naturally encoded amino acids. Those of skill in the art will recognize that several naturally encoded amino acids include reactive groups capable of conjugation to a payload or to a linker. These reactive groups include cysteine side chains, lysine side chains, and amino-terminal groups. In these embodiments, the antibody conjugate can comprise a payload or linker linked to the residue of an antibody reactive group. In these embodiments, the payload precursor or linker precursor comprises a reactive group capable of forming a bond with an antibody reactive group. Typical reactive groups include maleimide groups, activated carbonates (including but not limited to, p-nitrophenyl ester), activated esters (including but not limited to, N-hydroxysuccinimide, p-nitrophenyl ester, and aldehydes). Particularly useful reactive groups include maleimide and succinimide, for instance N-hydroxysuccinimide, for forming bonds to cysteine and lysine side chains. Additional reactive groups include alkynes, for example strained alkynes, and azides, for forming bonds to non-natural amino acids incorporated in antibody polypeptide chains. Further reactive groups are described in the sections and examples below.

In certain embodiments, the antibody comprises one or more modified amino acids having a reactive group, as described herein. Typically, the modified amino acid is not a naturally encoded amino acid. These modified amino acids can comprise a reactive group useful for forming a covalent bond to a linker precursor or to a payload precursor. One of skill in the art can use the reactive group to link the polypeptide to any molecular entity capable of forming a covalent bond to the modified amino acid. Thus, provided herein are conjugates comprising an antibody comprising a modified amino acid residue linked to a payload directly or indirectly via a linker. Exemplary modified amino acids are described in the sections below. Generally, the modified amino acids have reactive groups capable of forming bonds to linkers or payloads with complementary reactive groups.

The non-natural amino acids are positioned at select locations in a polypeptide chain of the antibody. These locations were identified as providing optimum sites for substitution with the non-natural amino acids. Each site is capable of bearing a non-natural amino acid with optimum structure, function and/or methods for producing the antibody.

In certain embodiments, a site-specific position for substitution provides an antibody that is stable. Stability can be measured by any technique apparent to those of skill in the art.

In certain embodiments, a site-specific position for substitution provides an antibody that has optimal functional properties. For instance, the antibody can show little or no loss of binding affinity for its target antigen compared to an antibody without the site-specific non-natural amino acid. In certain embodiments, the antibody can show enhanced binding compared to an antibody without the site-specific non-natural amino acid.

In certain embodiments, a site-specific position for substitution provides an antibody that can be made advantageously. For instance, in certain embodiments, the antibody shows advantageous properties in its methods of synthesis, discussed below. In certain embodiments, the antibody can show little or no loss in yield in production compared to an antibody without the site-specific non-natural amino acid. In certain embodiments, the antibody can show enhanced yield in production compared to an antibody without the site-specific non-natural amino acid. In certain embodiments, the antibody can show little or no loss of tRNA suppression compared to an antibody without the site-specific non-natural amino acid. In certain embodiments, the antibody can show enhanced tRNA suppression in production compared to an antibody without the site-specific non-natural amino acid.

In certain embodiments, a site-specific position for substitution provides an antibody that has advantageous solubility. In certain embodiments, the antibody can show little or no loss in solubility compared to an antibody without the site-specific non-natural amino acid. In certain embodiments, the antibody can show enhanced solubility compared to an antibody without the site-specific non-natural amino acid.

In certain embodiments, a site-specific position for substitution provides an antibody that has advantageous expression. In certain embodiments, the antibody can show little or no loss in expression compared to an antibody without the site-specific non-natural amino acid. In certain embodiments, the antibody can show enhanced expression compared to an antibody without the site-specific non-natural amino acid.

In certain embodiments, a site-specific position for substitution provides an antibody that has advantageous folding. In certain embodiments, the antibody can show little or no loss in proper folding compared to an antibody without the site-specific non-natural amino acid. In certain embodiments, the antibody can show enhanced folding compared to an antibody without the site-specific non-natural amino acid.

In certain embodiments, a site-specific position for substitution provides an antibody that is capable of advantageous conjugation. As described below, several non-natural amino acids have side chains or functional groups that facilitate conjugation of the antibody to a second agent, either directly or via a linker. In certain embodiments, the antibody can show enhanced conjugation efficiency compared to an antibody without the same or other non-natural amino acids at other positions. In certain embodiments, the antibody can show enhanced conjugation yield compared to an antibody without the same or other non-natural amino acids at other positions. In certain embodiments, the antibody can show enhanced conjugation specificity compared to an antibody without the same or other non-natural amino acids at other positions.

The one or more non-natural amino acids are located at selected site-specific positions in at least one polypeptide chain of the antibody. The polypeptide chain can be any polypeptide chain of the antibody without limitation, including either light chain or either heavy chain. The site-specific position can be in any domain of the antibody, including any variable domain and any constant domain.

In certain embodiments, the antibodies provided herein comprise one non-natural amino acid at a site-specific position. In certain embodiments, the antibodies provided herein comprise two non-natural amino acids at site-specific positions. In certain embodiments, the antibodies provided herein comprise three non-natural amino acids at site-specific positions. In certain embodiments, the antibodies provided herein comprise more than three non-natural amino acids at site-specific positions. In certain embodiments, the antibodies provided herein comprise four non-natural amino acids at site-specific positions.

In certain embodiments, the antibodies provided herein comprise one or more non-natural amino acids each at a position selected from the group consisting of heavy chain or light chain residues HC—F404, HC—K121, HC—Y180, HC—F241, HC-221, HC—Y391, LC-T22, LC-S7, LC-N152, LC-K42, LC-E161, LC-D170, HC—S136, HC—S25, HC-A40, HC—S119, HC—S190, HC—K222, HC—R19, HC—Y52, or HC—S70 according to the Kabat or Chothia or EU numbering scheme, or a post-translationally modified variant thereof. In certain embodiments, the antibodies provided herein comprise one or more non-natural amino acids each at a position selected from the group consisting of heavy chain or light chain residues HC—F404, HC—Y180, HC—F241, HC—Y391, LC-K42, and LC-E161, according to the Kabat or Chothia or EU numbering scheme, or a post-translationally modified variant thereof. In certain embodiments, the antibodies provided herein comprise one or more non-natural amino acids each at a position selected from the group consisting of heavy chain or light chain residues HC—F404, HC—Y180, LC-K42, and LC-E161, according to the Kabat or Chothia or EU numbering scheme, or a post-translationally modified variant thereof. In certain embodiments, the antibodies provided herein comprise one or more non-natural amino acids each at a position selected from the group consisting of heavy chain or light chain residues HC—F404, HC—Y180, HC—Y391, HC—F241, and LC-K42, according to the Kabat or Chothia or EU numbering scheme, or a post-translationally modified variant thereof. In certain embodiments, the antibodies provided herein comprise a non-natural amino acid at position HC—F404 according to the Kabat or Chothia or EU numbering scheme, or a post-translationally modified variant thereof. In certain embodiments, the antibodies provided herein comprise a non-natural amino acid at position HC—Y180, according to the Kabat or Chothia or EU numbering scheme, or a post-translationally modified variant thereof. In certain embodiments, the antibodies provided herein comprise non-natural amino acids at positions HC—F404 and HC—Y180, according to the Kabat or Chothia or EU numbering scheme, or a post-translationally modified variant thereof. In certain embodiments, the antibodies provided herein comprise a non-natural amino acid at position HC—F241 according to the Kabat or Chothia or EU numbering scheme, or a post-translationally modified variant thereof. In certain embodiments, the antibodies provided herein comprise a non-natural amino acid at position LC-K42 according to the Kabat or Chothia or EU numbering scheme, or a post-translationally modified variant thereof. In certain embodiments, the antibodies provided herein comprise a non-natural amino acid at position LC-E161 according to the Kabat or Chothia or EU numbering scheme, or a post-translationally modified variant thereof. In certain embodiments, the antibodies provided herein comprise non-natural amino acids at positions HC—F404, HC—Y180, and LC-K42, according to the Kabat or Chothia or EU numbering scheme, or a post-translationally modified variant thereof. In certain embodiments, the antibodies provided herein comprise non-natural amino acids at positions HC—F404, HC—Y180, LC-K42, and LC-E161, according to the Kabat or Chothia or EU numbering scheme, or a post-translationally modified variant thereof. In certain embodiments, the antibodies provided herein comprise non-natural amino acids at positions HC—F404, HC—Y180, and HC—F241, according to the Kabat or Chothia or EU numbering scheme, or a post-translationally modified variant thereof. In certain embodiments, the antibodies provided herein comprise non-natural amino acids at positions HC—F404, HC—Y180, HC—F241, and LC-K42, according to the Kabat or Chothia or EU numbering scheme, or a post-translationally modified variant thereof. In certain embodiments, the antibodies provided herein comprise non-natural amino acids at positions HC—F404, HC—Y180, HC—F241, and LC-K42, according to the Kabat or Chothia or EU numbering scheme, or a post-translationally modified variant thereof. In certain embodiments, the antibodies provided herein comprise non-natural amino acids at positions HC—F241 and HC—F404, according to the Kabat or Chothia or EU numbering scheme, or a post-translationally modified variant thereof. In certain embodiments, the antibodies provided herein comprise non-natural amino acids at positions HC—Y180, HC—F404, LC-K42 and LC-E161, according to the Kabat or Chothia or EU numbering scheme, or a post-translationally modified variant thereof. In certain embodiments, the antibodies provided herein comprise non-natural amino acids at positions HC—Y180, HC—F241, HC—F404 and HC—Y391, according to the Kabat or Chothia or EU numbering scheme, or a post-translationally modified variant thereof. In these designations, HC indicates a heavy chain residue, and LC indicates a light chain residue.

In some aspects, the present disclosure provides antibody conjugates according to the following formula:

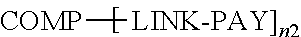

or a pharmaceutically acceptable salt, solvate, stereoisomer, regioisomer, or tautomer thereof, wherein:
COMP is a residue of an anti-Tissue Factor antibody;
   i. PAY is a payload moiety;
   ii. LINK is a linker; and
   iii. n2 is an integer from 1 to 10,
   iv, wherein COMP comprises one or more non-natural amino acids.

In certain embodiments, provided herein are conjugates according to Formula (C1) or (C2):

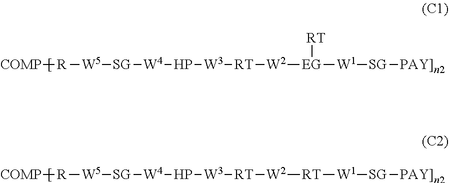

or a pharmaceutically acceptable salt, solvate, stereoisomer, regioisomer, or tautomer thereof, wherein:
   COMP is a residue of an anti-Tissue Factor antibody;
   PAY is a payload moiety;
   W¹, W², W³, W⁴, and W⁵ are each independently a single bond, absent, or a divalent attaching group;
   EG is absent, or an eliminator group;
   each RT is a release trigger group, in the backbone of Formula (C1) or (C2) or bonded to EG, wherein each RT is optional;
   HP is a single bond, absent, or a divalent hydrophilic group;
   each SG is a single bond, absent, or a divalent spacer group;
   R is hydrogen, a terminal conjugating group, or a divalent residue of a terminal conjugating group; and
   n2 is an integer from 1 to 10.

In some embodiments, a conjugate according to Formula (C1) or (C2) comprises n2 number of linked PAY moieties, wherein n2 is an integer from 1 to 10. In some embodiments, n2 is 2. In some embodiments, n2 is 3. In some embodiments, n2 is 4. In some embodiments, n2 is 5. In some embodiments, n2 is 6. In some embodiments, n2 is 7. In some embodiments, n2 is 8. In some embodiments, n2 is 10.

### 1. Attaching Groups

Attaching groups facilitate incorporation of eliminator groups, release trigger groups, hydrophobic groups, spacer groups, and/or conjugating groups into a compound. Useful attaching groups are known to, and are apparent to, those of skill in the art. Examples of useful attaching groups are provided herein. In certain embodiments, attaching groups are designated W¹, W², W³, W⁴, or W⁵. In certain embodiments, an attaching group can comprise a divalent ketone, divalent ester, divalent ether, divalent amide, divalent amine, alkylene, arylene, sulfide, disulfide, carbonylene, or a combination thereof. In certain embodiments an attaching group can comprise —C(O)—, —O—, —C(O)NH—, —C(O)NH-alkyl-, —OC(O)NH—, —SC(O)NH—, —NH—, —NH-alkyl-, —N(CH₃)CH₂CH₂N(CH₃)—, —S—, —S—S—, —OCH₂CH₂O—, or the reverse (e.g. —NHC(O)—) thereof, or a combination thereof.

### 2. Eliminator Groups

Eliminator groups facilitate separation of a biologically active portion of a compound or conjugate described herein from the remainder of the compound or conjugate in vivo and/or in vitro. Eliminator groups can also facilitate separation of a biologically active portion of a compound or conjugate described herein in conjunction with a release trigger group. For example, the eliminator group and the release trigger group can react in a Releasing Reaction to release a biologically active portion of a compound or conjugate described herein from the compound or conjugate in vivo and/or in vitro. Upon initiation of the Releasing Reaction by the release trigger, the eliminator group cleaves the biologically active moiety, or a prodrug form of the biologically active moiety, and forms a stable, non-toxic entity that has no further effect on the activity of the biologically active moiety.

In certain embodiments, the eliminator group is designated EG herein. Useful eliminator groups include those described herein. In certain embodiments, the eliminator group is:

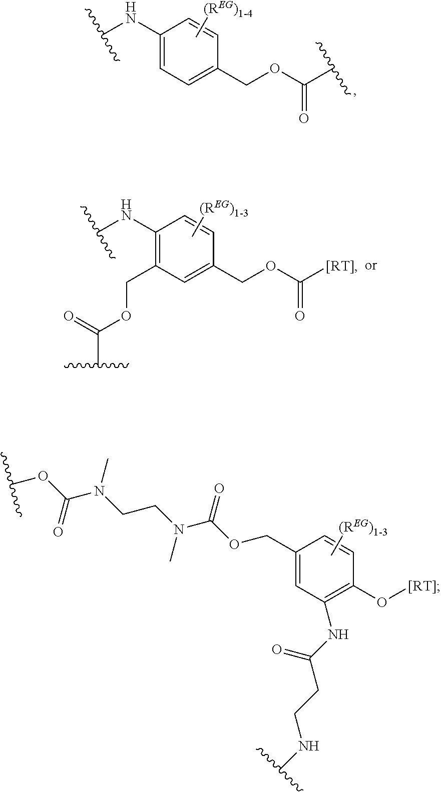

wherein R^{EG} is selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—. In each structure, the phenyl ring can be bound to one, two, three, or in some cases, four R^{EG} groups. In the second and third structures, those of skill will recognize that EG is bonded to an RT that is not within the backbone of formula (C1) as indicated in the above description of formula (C1). In some embodiments, R^{EG} is selected from the group consisting of hydrogen, alkyl, biphenyl, —CF₃, alkoxyl, alkylamino, dialkylamino, alkyl-C(O)O—, alkylamino-C(O)— and dialkylaminoC(O)—. In further embodiments, R^{EG} is selected from the group consisting of hydrogen, —NO₂, —CN, fluoro, bromo, and chloro. In certain embodiments, the eliminator group is

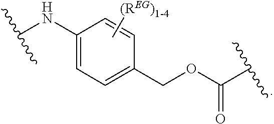

In certain embodiments, the eliminator group is

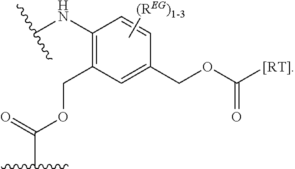

In certain embodiments, the eliminator group is

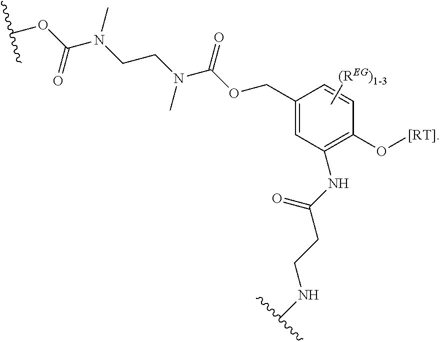

In some embodiments, provided herein is a conjugate according to Formula (C1) or (C2) or a pharmaceutically acceptable salt, solvate, stereoisomer, or tautomer thereof; wherein EG comprises phenylene, carboxylene, amine, or a combination thereof. In some embodiments, the eliminator group is:

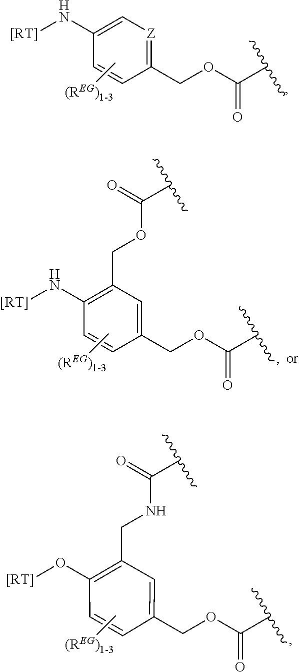

wherein Z may be CH or N, R^{EG} is selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—. In each structure, the phenyl ring can be bound to one, two, three, or in some cases, four R^{EG} groups. In the first and second structures, those of skill will recognize that EG is bonded to an RT that is not within the backbone of formula (C1) as indicated in the above description of formula (C1). In some embodiments, R^{EG} is selected from the group consisting of hydrogen, alkyl, biphenyl, —CF₃, alkoxyl, alkylamino, dialkylamino, alkyl-C(O)O—, alkylamino-C(O)— and dialkylaminoC(O)—. In further embodiments, R^{EG} is selected from the group consisting of hydrogen, —NO₂, —CN, fluoro, bromo, and chloro. In some embodiments, each R^{EG} in the EG is hydrogen. In certain embodiments, the eliminator group is

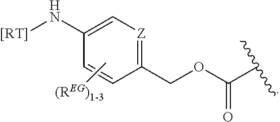

In certain embodiments, the eliminator group is

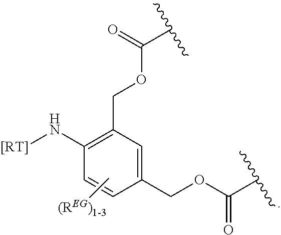

In certain embodiments, the eliminator group is

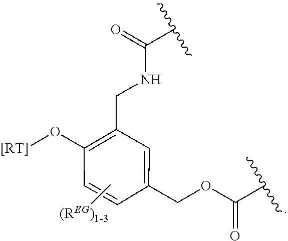

### 3. Release Trigger Groups

Release trigger groups facilitate separation of a biologically active portion of a compound or conjugate described herein from the remainder of the compound or conjugate in vivo and/or in vitro. Release trigger groups can also facilitate separation of a biologically active portion of a compound or conjugate described herein in conjunction with an eliminator group. For example, the eliminator group and the release trigger group can react in a Releasing Reaction to release a biologically active portion of a compound or conjugate described herein from the compound or conjugate in vivo and/or in vitro. In certain embodiments, the release trigger can act through a biologically-driven reaction with high tumor: nontumor specificity, such as the proteolytic action of an enzyme overexpressed in a tumor environment.

In certain embodiments, the release trigger group is designated RT herein. In certain embodiments, RT is divalent and bonded within the backbone of formula (C1). In other embodiments, RT is monovalent and bonded to EG as depicted above. Useful release trigger groups include those described herein. In certain embodiments, the release trigger group comprises a residue of a natural or non-natural amino acid or residue of a sugar ring. In certain embodiments, the release trigger group is:

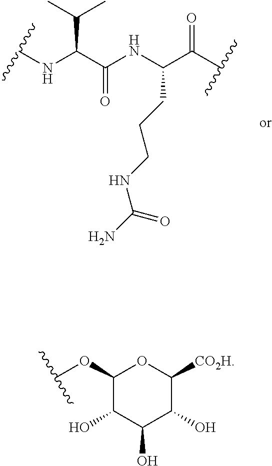 [SMILES: *N[C@H](C(=O)N[C@@H](CCCNC(N)=O)C(*)=O)C(C)C ; *O[C@H]1O[C@@H](C(=O)O)[C@H](O)[C@@H](O)[C@@H]1O]

Those skilled in the art will recognize that the first structure is divalent and can be bonded within the backbone of Formula (C1) or as depicted in Formula (C2), and that the second structure is monovalent and can be bonded to EG as depicted in formula (C1) above. In certain embodiments, the release trigger group is

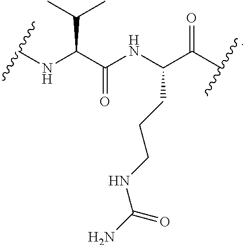 [SMILES: *N[C@H](C(=O)N[C@@H](CCCNC(N)=O)C(*)=O)C(C)C]

In certain embodiments, the release trigger group is

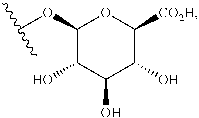 [SMILES: *O[C@H]1O[C@@H](C(=O)O)[C@H](O)[C@@H](O)[C@@H]1O]

In some embodiments, the release trigger group is a protease-cleavable R₁-Val-X peptide having the structure of:

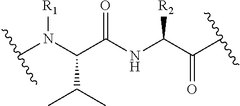

wherein R₁ is H or

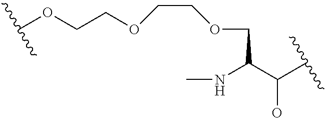

and R₂ is CH₃, CH₂CH₂CO₂H, or (CH₂)₃NHCONH₂; a legumain-cleavable Ala-Ala-Asn or Ala-Ala-Asp peptide having the structure of:

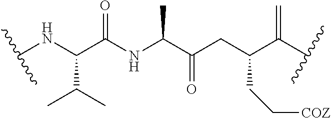

where Z is OH or NH₂; or a β-glucuronidase-cleavable β-glucuronide having the structure of:

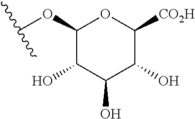 [SMILES: *O[C@H]1O[C@@H](C(=O)O)[C@H](O)[C@@H](O)[C@@H]1O]

or a Val-Lys-Gly peptide having the structure of:

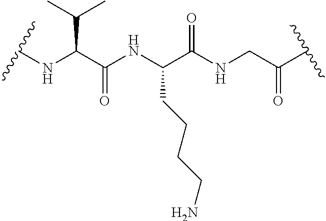 [SMILES: *N[C@H](C(=O)N[C@@H](CCCCN)C(=O)NCC(*)=O)C(C)C]

Those of skill will recognize that

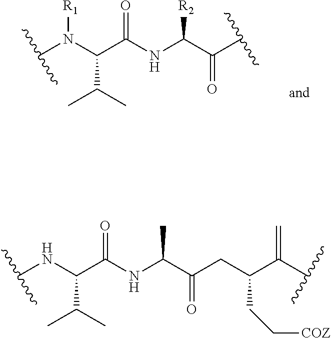

are divalent structures and can be bonded within the backbone of Formula (C1) or as depicted in Formula (C2). The structure

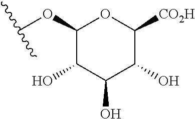 [SMILES: *O[C@H]1O[C@@H](C(=O)O)[C@H](O)[C@@H](O)[C@@H]1O]

is monovalent and can be bonded to EG as depicted in formula (C1) above.

In certain embodiments, the release trigger is selected from the group consisting of Val-Lys-Gly, Val-Ala-Asp, Ala-Ala-Ala, Val-Lys, Gly-Gly-Gly, Val-Ala, Gly-Gly-Phe-Gly, Val-Cit, Val-Cit, Val-Glu, Ala-Ala-Asn, and Gly. In certain embodiments, the release trigger further comprises a non-natural amino acid. In certain embodiments, the non-natural amino acid is according to

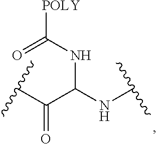

where POLY is a polymer, for instance a hydrophilic polymer. In certain embodiments, the non-natural amino acid is according to

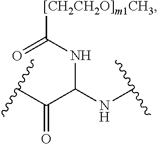

where m1 is an integer from 1 to 25, for instance 8, 9, 10, 11, 12, 13, 14, 15, 20, or 25.

### 4. Hydrophilic Groups

Hydrophilic groups facilitate increasing the hydrophilicity of the compounds described herein. It is believed that increased hydrophilicity allows for greater solubility in aqueous solutions, such as aqueous solutions found in biological systems. Hydrophilic groups can also function as spacer groups, which are described in further detail herein.

In certain embodiments, the hydrophilic group is designated HP herein. Useful hydrophilic groups include those described herein. In certain embodiments, the hydrophilic group is a divalent poly (ethylene glycol). In certain embodiments, the hydrophilic group is a divalent poly (ethylene glycol) according to the formula:

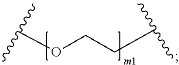

wherein m1 is an integer from 1 to 13, optionally 1 to 4, optionally 2 to 4, or optionally 4 to 8. In certain embodiments, m1 is 4. In certain embodiments, m1 is 12. In certain embodiments, m1 is 13.

In some embodiments, the hydrophilic group is a divalent poly (ethylene glycol) having the following formula:

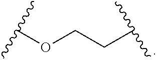 [SMILES: *CCO*]

In some other embodiments, the hydrophilic group is a divalent poly (ethylene glycol) having the following formula:

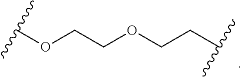 [SMILES: *CCOCCO*]

In other embodiments, the hydrophilic group is a divalent poly (ethylene glycol) having the following formula:

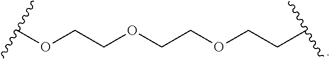 [SMILES: *CCOCCOCCO*]

In other embodiments, the hydrophilic group is a divalent poly (ethylene glycol) having the following formula:

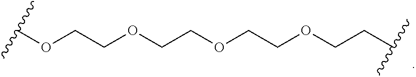 [SMILES: *CCOCCOCCOCCO*]

In some embodiments, the hydrophilic group is sulfonic acid. In some embodiments, the hydrophilic group is the side chain of cysteic acid. In some embodiments, the hydrophilic group can bear a chain-presented sulfonic acid having the formula:

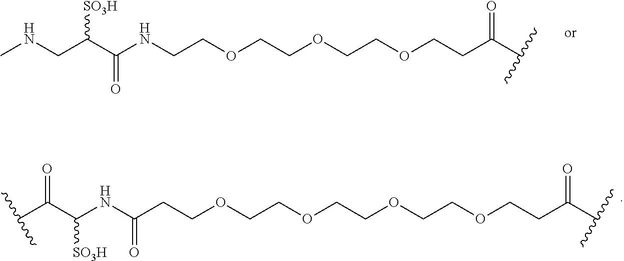 [SMILES: *C(=O)CCOCCOCCOCCNC(=O)C(CNC)S(=O)(=O)O ; *C(=O)CCOCCOCCOCCOCCC(=O)NC(C(*)=O)S(=O)(=O)O]

(a) In certain embodiments, the hydrophilic group is the side chain of a non-natural amino acid according to

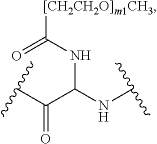

where m1 is an integer from 1 to 25, for instance 8, 9, 10, 11, 12, 13, 14, 15, 20, or 25.

### 5. Spacer Groups

Spacer groups facilitate spacing of the conjugating group from the other groups of the compounds described herein. This spacing can lead to more efficient conjugation of the compounds described herein to an antibody as well as more efficient cleavage of the active catabolite. The spacer group can also stabilize the conjugating group and lead to improved overall antibody-drug conjugate properties.

In certain embodiments, the spacer group is designated SP herein. Useful spacer groups include those described herein. In certain embodiments, the spacer group is:

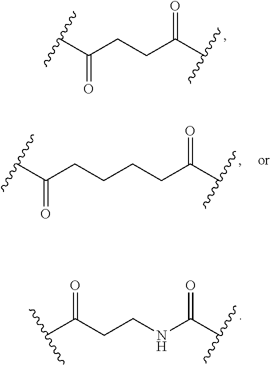 [SMILES: *C(=O)CCC(*)=O ; *C(=O)CCCCC(*)=O ; *C(=O)CCNC(*)=O]

In certain embodiments, the spacer group, W⁴, and the hydrophilic group combine to form a divalent poly (ethylene glycol) according to the formula:

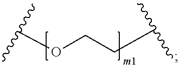

wherein m1 is an integer from 1 to 13, optionally 1 to 4, optionally 2 to 4, or optionally 4 to 8.

In some embodiments, the SP is

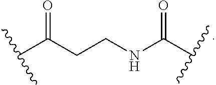 [SMILES: *C(=O)CCNC(*)=O]

In some embodiments, the divalent poly (ethylene glycol) has the following formula:

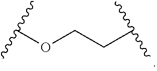 [SMILES: *CCO*]

In some other embodiments, the divalent poly (ethylene glycol) has the following formula:

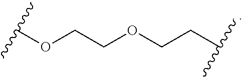 [SMILES: *CCOCCO*]

In other embodiments, the divalent poly (ethylene glycol) has the following formula:

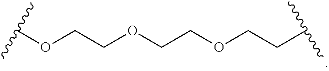 [SMILES: *CCOCCOCCO*]

In other embodiments, the divalent poly (ethylene glycol) has the following formula:

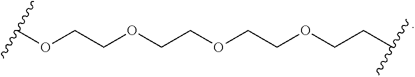 [SMILES: *CCOCCOCCOCCO*]

In some embodiments, the spacer group can bear a chain-presented sulfonic acid having the formula:

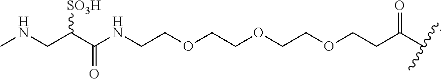 [SMILES: *C(=O)CCOCCOCCOCCNC(=O)C(CNC)S(=O)(=O)O]

In some embodiments, the spacer group is a diamine. In some embodiments, the spacer group is according to

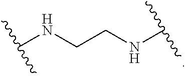 [SMILES: *NCCN*]

In some embodiments, the spacer group comprises fused rings or spiro rings. In some embodiments, the spacer group is according to

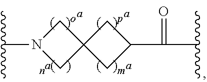

where each n^{a}, m^{a}, o^{a}, and p^{a} is an integer independently selected from 1, 2, 3, 4, and 5. In some embodiments, the spacer group is according to

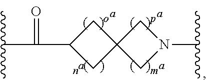

where each n^{a}, m^{a}, o^{a}, and p^{a} is an integer independently selected from 1, 2, 3, 4, and 5. In some embodiments, the spacer group is selected from the group consisting of:

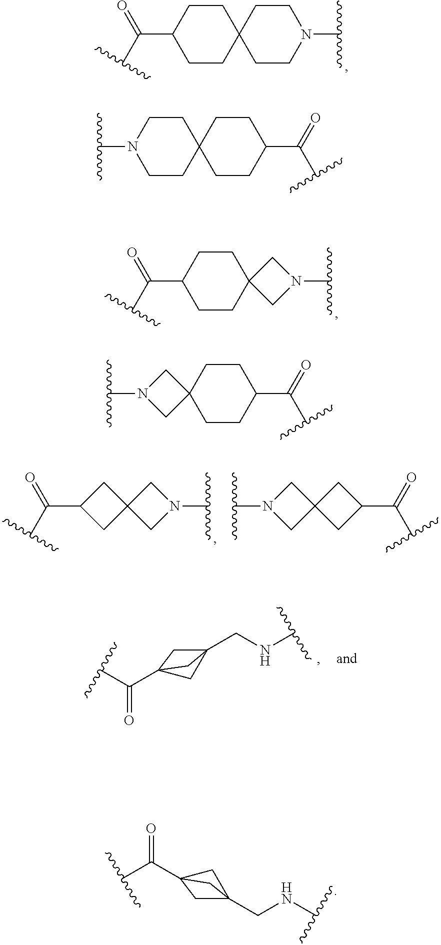

### 6. Conjugating Groups and Residues Thereof

Conjugating groups facilitate conjugation of the payloads described herein to a second compound, such as an antibody described herein. In certain embodiments, the conjugating group is designated R herein. Conjugating groups can react via any suitable reaction mechanism known to those of skill in the art. In certain embodiments, a conjugating group reacts through a [3+2] alkyne-azide cycloaddition reaction, inverse-electron demand Diels-Alder ligation reaction, thiol-electrophile reaction, or carbonyl-oxyamine reaction, as described in detail herein. In certain embodiments, the conjugating group comprises an alkyne, strained alkyne, tetrazine, thiol, para-acetyl-phenylalanine residue, oxyamine, maleimide, or azide. In certain embodiments, the conjugating group is:

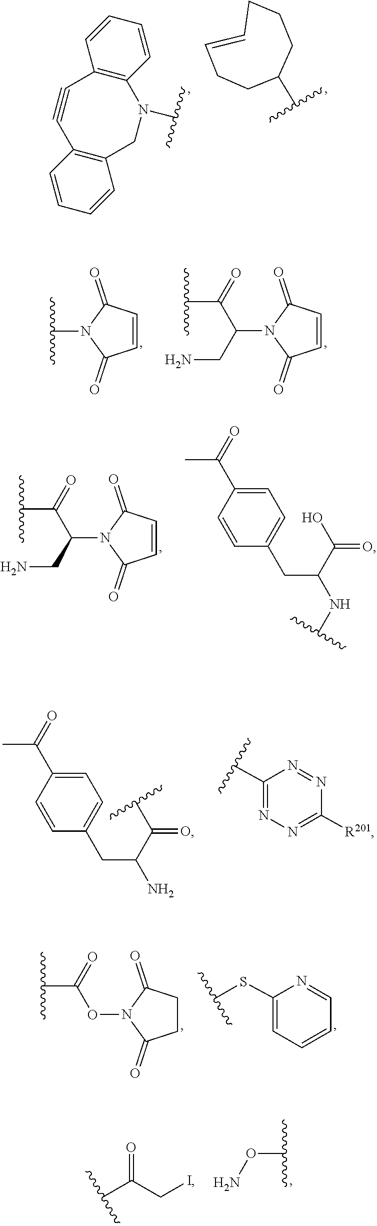

—N₃, or —SH; wherein R²⁰¹ is lower alkyl. In an embodiment, R²⁰¹ is methyl, ethyl, or propyl. In an embodiment, R²⁰¹ is methyl. Additional conjugating groups are described in, for example, U.S. Patent Publication No. 2014/0356385, U.S. Patent Publication No. 2013/0189287, U.S. Patent Publication No. 2013/0251783, U.S. Pat. Nos. 8,703,936, 9,145,361, 9,222,940, and 8,431,558.

After conjugation, a divalent residue of the conjugating group is formed and is bonded to the residue of an antibody. The structure of the divalent residue is determined by the type of conjugation reaction employed to form the conjugate.

In certain embodiments when a conjugate is formed through a [3+2] alkyne-azide cycloaddition reaction, the divalent residue of the conjugating group comprises a triazole ring or fused cyclic group comprising a triazole ring. In certain embodiment when a conjugate is formed through a strain-promoted [3+2] alkyne-azide cycloaddition (SPAAC) reaction, the divalent residue of the conjugating group is:

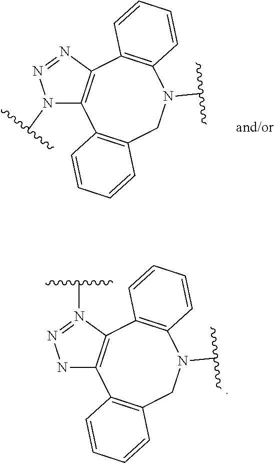

In certain embodiments when a conjugate is formed through a tetrazine inverse electron demand Diels-Alder ligation reaction, the divalent residue of the conjugating group comprises a fused bicyclic ring having at least two adjacent nitrogen atoms in the ring. In certain embodiments when a conjugate is formed through a tetrazine inverse electron demand Diels-Alder ligation reaction, the divalent residue of the conjugating group is:

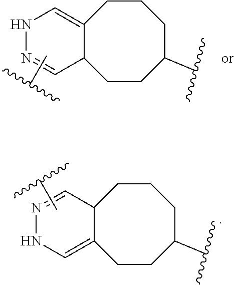

In certain embodiments when a conjugate is formed through a thiol-maleimide reaction, the divalent residue of the conjugating group comprises succinimidylene and a sulfur linkage. In certain embodiments when a conjugate is formed through a thiol-maleimide reaction, the divalent residue of the conjugating group is:

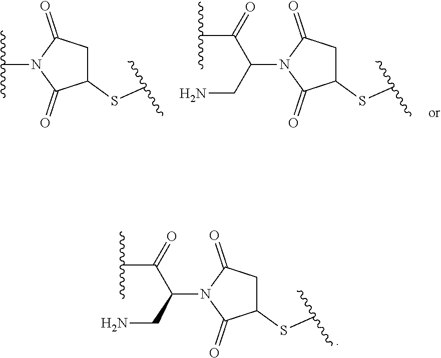 [SMILES: *SC1CC(=O)N(*)C1=O ; *SC1CC(=O)N(C(CN)C(*)=O)C1=O ; *SC1CC(=O)N([C@@H](CN)C(*)=O)C1=O]

In certain embodiments, a conjugate is formed through a thiol-N-hydroxysuccinimide reaction using the following group:

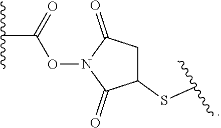 [SMILES: *SC1CC(=O)N(OC(*)=O)C1=O]

The reaction involved for formation of the conjugate comprises the following step:

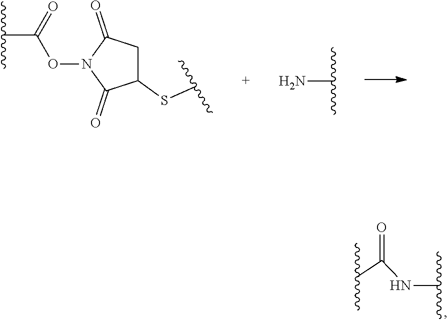

and the resulting divalent residue of the conjugating group is:

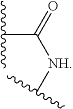 [SMILES: *NC(*)=O]

In certain embodiments when a conjugate is formed through a carbonyl-oxyamine reaction, the divalent residue of the conjugating group comprises a divalent residue of a non-natural amino acid. In certain embodiments when a conjugate is formed through a carbonyl-oxyamine reaction, the divalent residue of the conjugating group is:

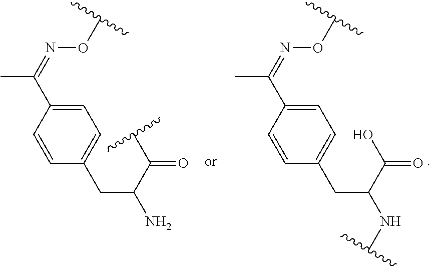 [SMILES: *O/N=C(/C)c1ccc(CC(N)C(*)=O)cc1 ; *NC(Cc1ccc(/C(C)=N\O*)cc1)C(=O)O]

In certain embodiments when a conjugate is formed through a carbonyl-oxyamine reaction, the divalent residue of the conjugating group comprises an oxime linkage. In certain embodiments when a conjugate is formed through a carbonyl-oxyamine reaction, the divalent residue of the conjugating group is:

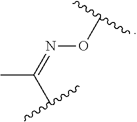 [SMILES: *O/N=C(\*)C]

In an embodiment, provided herein is a conjugate according to Formula (C1) or (C2), or a pharmaceutically acceptable salt, solvate, stereoisomer, or tautomer thereof; wherein R comprises a triazole ring. In an embodiment, provided herein is a conjugate according to Formula (C1) or (C2), or a pharmaceutically acceptable salt, solvate, stereoisomer, or tautomer thereof; wherein R is a triazole ring or fused cyclic group comprising a triazole ring. In an embodiment, provided herein is a conjugate according to Formula (C1) or (C2), or a pharmaceutically acceptable salt, solvate, stereoisomer, or tautomer thereof; wherein R is:

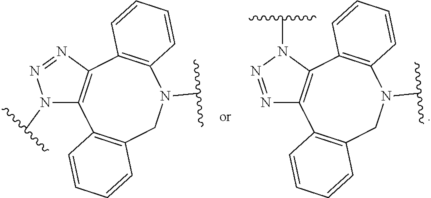 [SMILES: *N1Cc2ccccc2-c2c(nnn2*)-c2ccccc21 ; *N1Cc2ccccc2-c2nnn(*)c2-c2ccccc21]

In an embodiment, provided herein is a conjugate according to Formula (C1) or (C2), or a pharmaceutically acceptable salt, solvate, stereoisomer, or tautomer thereof; wherein R comprises a fused bicyclic ring having at least two adjacent nitrogen atoms in the ring. In an embodiment, provided herein is a conjugate according to Formula (C1) or (C2), or a pharmaceutically acceptable salt, solvate, stereoisomer, or tautomer thereof; wherein R is:

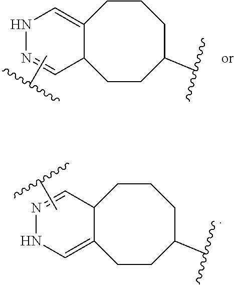

In an embodiment, provided herein is a conjugate according to Formula (C1) or (C2), or a pharmaceutically acceptable salt, solvate, stereoisomer, or tautomer thereof; wherein R comprises a sulfur linkage. In an embodiment, provided herein is a conjugate according to Formula (C1) or (C2), or a pharmaceutically acceptable salt, solvate, stereoisomer, or tautomer thereof; wherein R is:

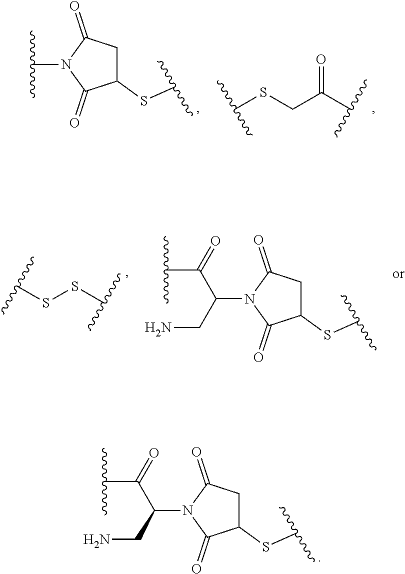

In an embodiment, provided herein is a conjugate according to Formula (C1) or (C2), or a pharmaceutically acceptable salt, solvate, stereoisomer, or tautomer thereof; wherein R comprises a divalent residue of a non-natural amino acid. In an embodiment, provided herein is a conjugate according to Formula (C1) or (C2), or a pharmaceutically acceptable salt, solvate, stereoisomer, or tautomer thereof; wherein R is:

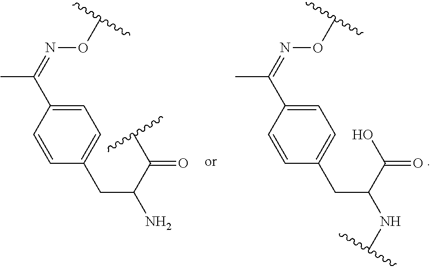 [SMILES: *O/N=C(/C)c1ccc(CC(N)C(*)=O)cc1 ; *NC(Cc1ccc(/C(C)=N\O*)cc1)C(=O)O]

In an embodiment, provided herein is a conjugate according to Formula (C1) or (C2), or a pharmaceutically acceptable salt, solvate, stereoisomer, or tautomer thereof; wherein comprises an amide linkage. In an embodiment, provided herein is a conjugate according to Formula (C1) or (C2), or a pharmaceutically acceptable salt, solvate, stereoisomer, or tautomer thereof; wherein R is:

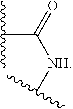 [SMILES: *NC(*)=O]

In an embodiment, provided herein is a conjugate according to Formula (C1) or (C2), or a pharmaceutically acceptable salt, solvate, stereoisomer, or tautomer thereof; wherein comprises an oxime linkage. In an embodiment, provided herein is a conjugate according to Formula (C1) or (C2), or a pharmaceutically acceptable salt, solvate, stereoisomer, or tautomer thereof; wherein R is:

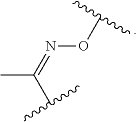 [SMILES: *O/N=C(\*)C]

In an embodiment, provided herein is a conjugate according to Formula (C1) or (C2), or a pharmaceutically acceptable salt, solvate, stereoisomer, or tautomer thereof; wherein R is:

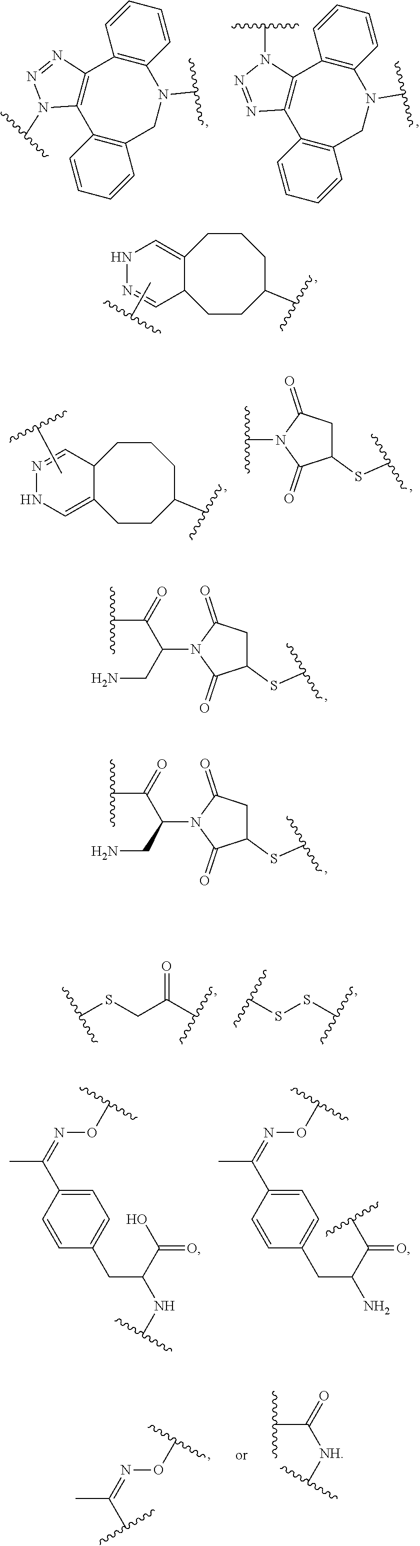

In an embodiment, provided herein is a compound according to Formula (C1) or (C2), or a pharmaceutically acceptable salt, solvate, stereoisomer, or tautomer thereof; wherein COMP is a residue of any compound known to be useful for conjugation to a payload, described herein, and an optional linker, described herein. In an embodiment, provided herein is a compound according to Formula (C1) or (C2), or a pharmaceutically acceptable salt, solvate, stereoisomer, or tautomer thereof; wherein COMP is a residue of an antibody chain.

In an aspect, provided herein is an antibody conjugate comprising payload, described herein, and an optional linker, described herein, linked to an anti-Tissue Factor antibody, wherein COMP is a residue of the antibody. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises a triazole ring or fused cyclic group comprising a triazole ring. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

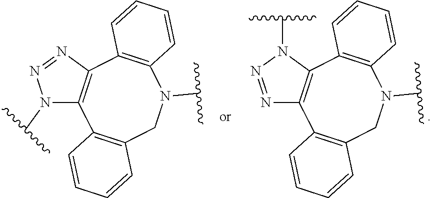 [SMILES: *N1Cc2ccccc2-c2c(nnn2*)-c2ccccc21 ; *N1Cc2ccccc2-c2nnn(*)c2-c2ccccc21]

In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises a fused bicyclic ring, wherein the fused bicyclic ring has at least two adjacent nitrogen atoms in the ring. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

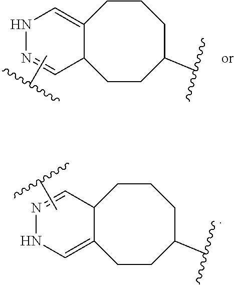

In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R comprises a sulfur linkage. In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R is:

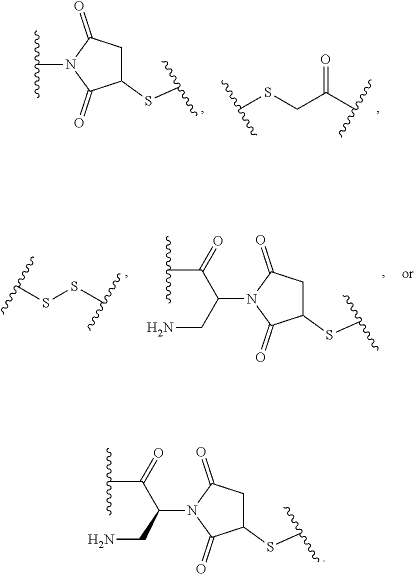

In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R comprises a divalent residue of a non-natural amino acid. In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R is:

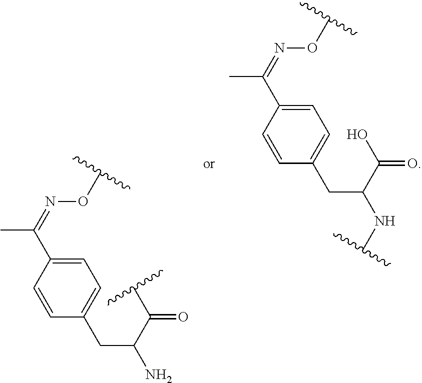 [SMILES: *O/N=C(/C)c1ccc(CC(N)C(*)=O)cc1 ; *NC(Cc1ccc(/C(C)=N\O*)cc1)C(=O)O]

In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R comprises an amide linkage. In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R is:

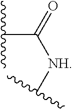 [SMILES: *NC(*)=O]

In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R comprises an amide linkage. In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R is:

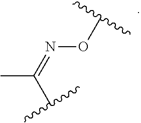 [SMILES: *O/N=C(\*)C]

In an aspect, provided herein is an antibody conjugate comprising a payload, described herein, and an optional linker, described herein, linked to an antibody according to Formula (C1) or (C2), or a pharmaceutically acceptable salt, solvate, stereoisomer, or tautomer thereof, wherein COMP is a residue of the antibody. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises a triazole ring or fused cyclic group comprising a triazole ring. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

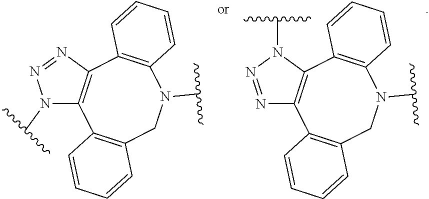 [SMILES: *N1Cc2ccccc2-c2c(nnn2*)-c2ccccc21 ; *N1Cc2ccccc2-c2nnn(*)c2-c2ccccc21]

In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises a fused bicyclic ring, wherein the fused bicyclic ring has at least two adjacent nitrogen atoms in the ring. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

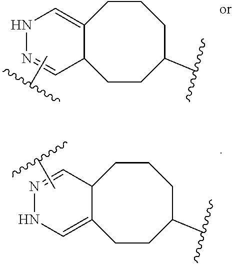

In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises a sulfur linkage. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

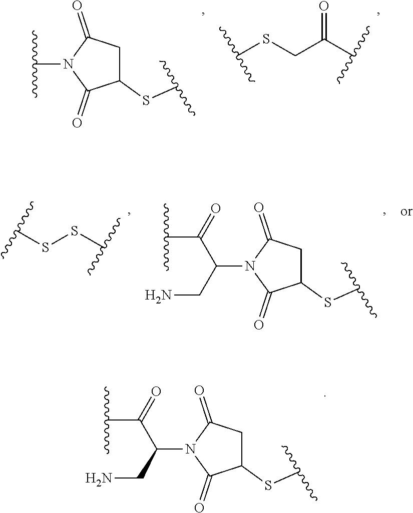

In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises a divalent residue of a non-natural amino acid. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

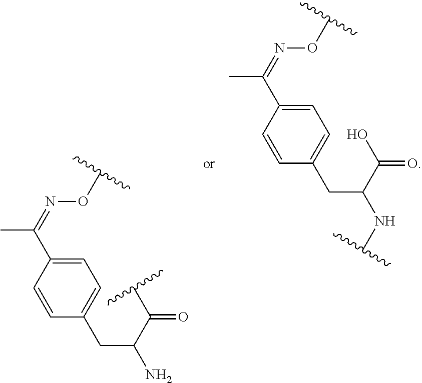 [SMILES: *O/N=C(/C)c1ccc(CC(N)C(*)=O)cc1 ; *NC(Cc1ccc(/C(C)=N\O*)cc1)C(=O)O]

In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises an amide linkage. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

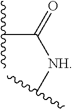 [SMILES: *NC(*)=O]

In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises an oxime linkage. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

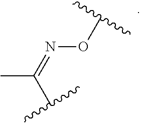 [SMILES: *O/N=C(\*)C]

In an aspect, provided herein is an antibody conjugate comprising a payload, described herein, and an optional linker, described herein, linked to an antibody chain according to Formula (C1) or (C2), or a pharmaceutically acceptable salt, solvate, stereoisomer, or tautomer thereof, wherein COMP is a residue of the antibody chain. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R comprises a triazole ring or fused cyclic group comprising a triazole ring. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R is:

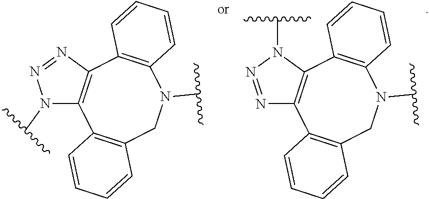 [SMILES: *N1Cc2ccccc2-c2c(nnn2*)-c2ccccc21 ; *N1Cc2ccccc2-c2nnn(*)c2-c2ccccc21]

In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R comprises a fused bicyclic ring, wherein the fused bicyclic ring has at least two adjacent nitrogen atoms in the ring. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R is:

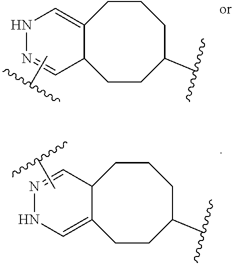

In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R comprises a sulfur linkage. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R is:

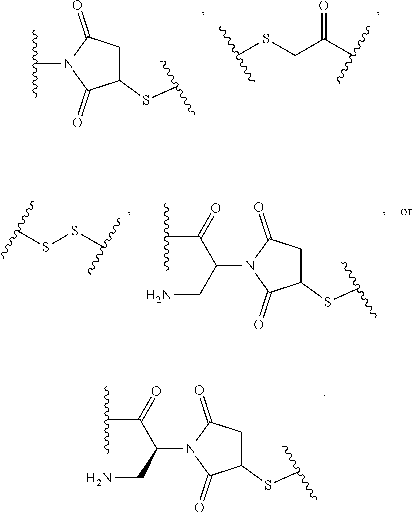

In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R comprises a divalent residue of a non-natural amino acid. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R is:

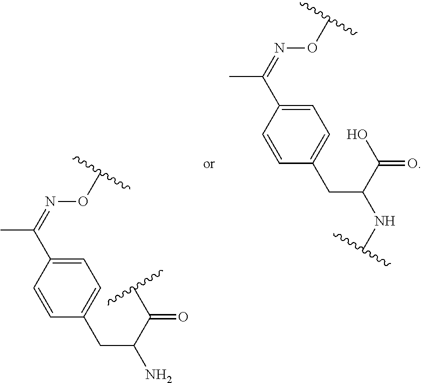 [SMILES: *O/N=C(/C)c1ccc(CC(N)C(*)=O)cc1 ; *NC(Cc1ccc(/C(C)=N\O*)cc1)C(=O)O]

In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R comprises an amide linkage. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R is:

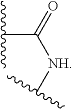 [SMILES: *NC(*)=O]

In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R comprises an amide linkage. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R is:

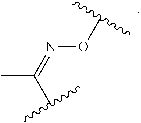 [SMILES: *O/N=C(\*)C]

### (b) Conjugates

In an embodiment, provided herein is a conjugate according to any of the following formulas, where COMP indicates a residue of the anti-Tissue Factor antibody and PAY indicates a payload moiety:

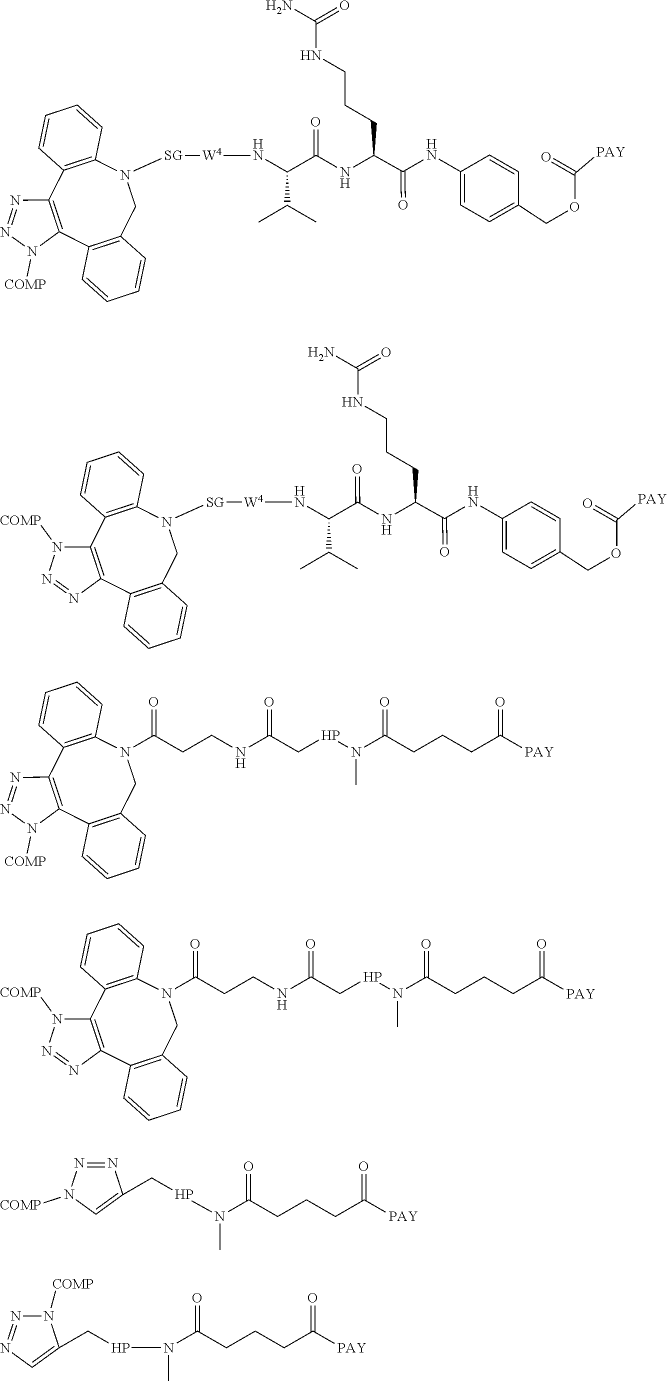

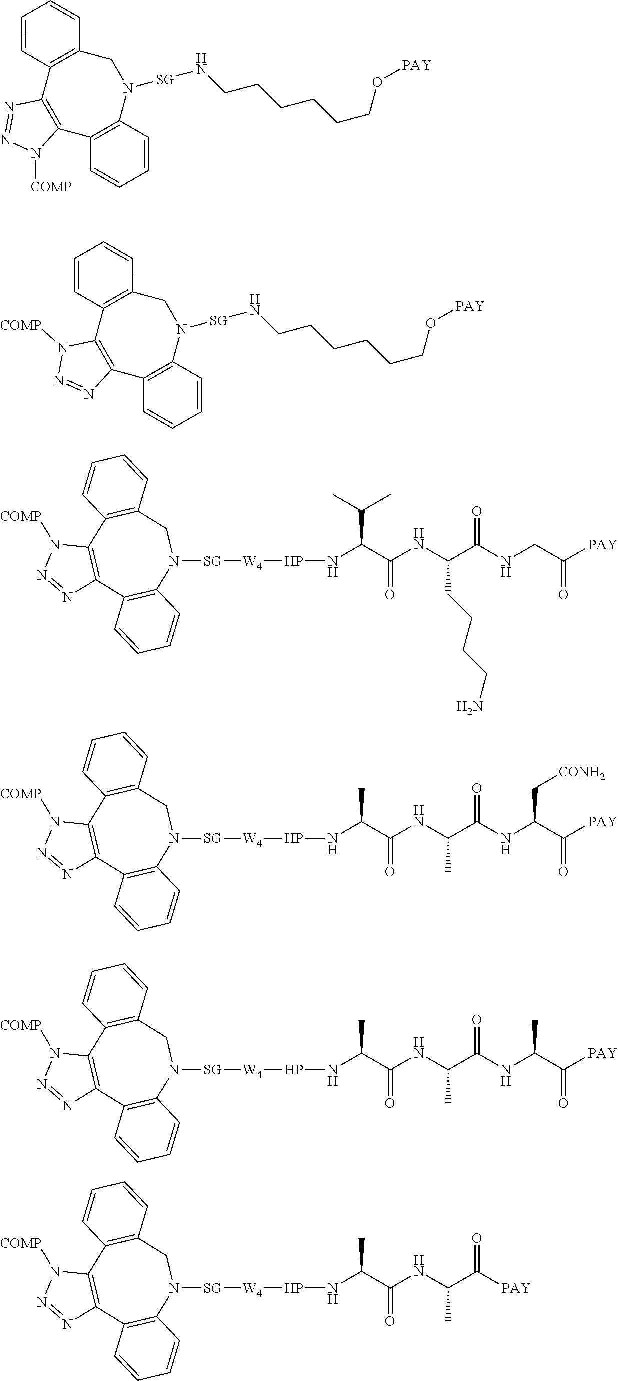

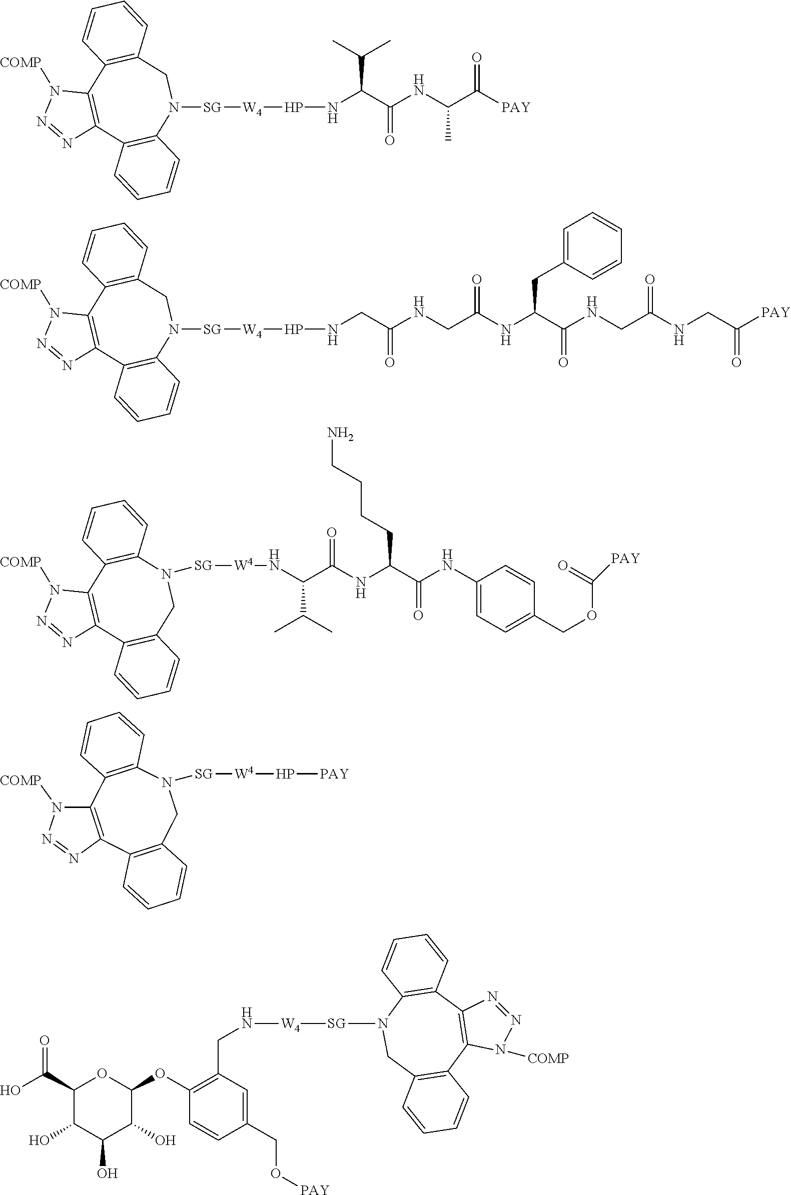

In an embodiment, provided herein is a conjugate according to any of the following formulas, where COMP indicates a residue of the anti-Tissue Factor antibody and PAY indicates a payload moiety:

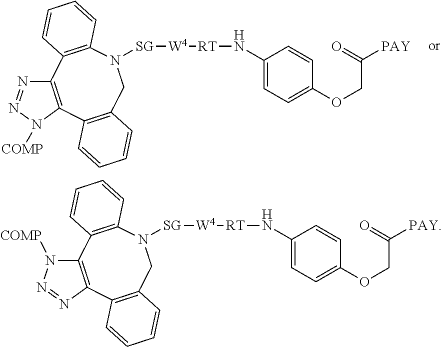

In an embodiment, provided herein is a conjugate according to any of the following formulas, where COMP indicates a residue of the anti-Tissue Factor antibody and PAY indicates a payload moiety:

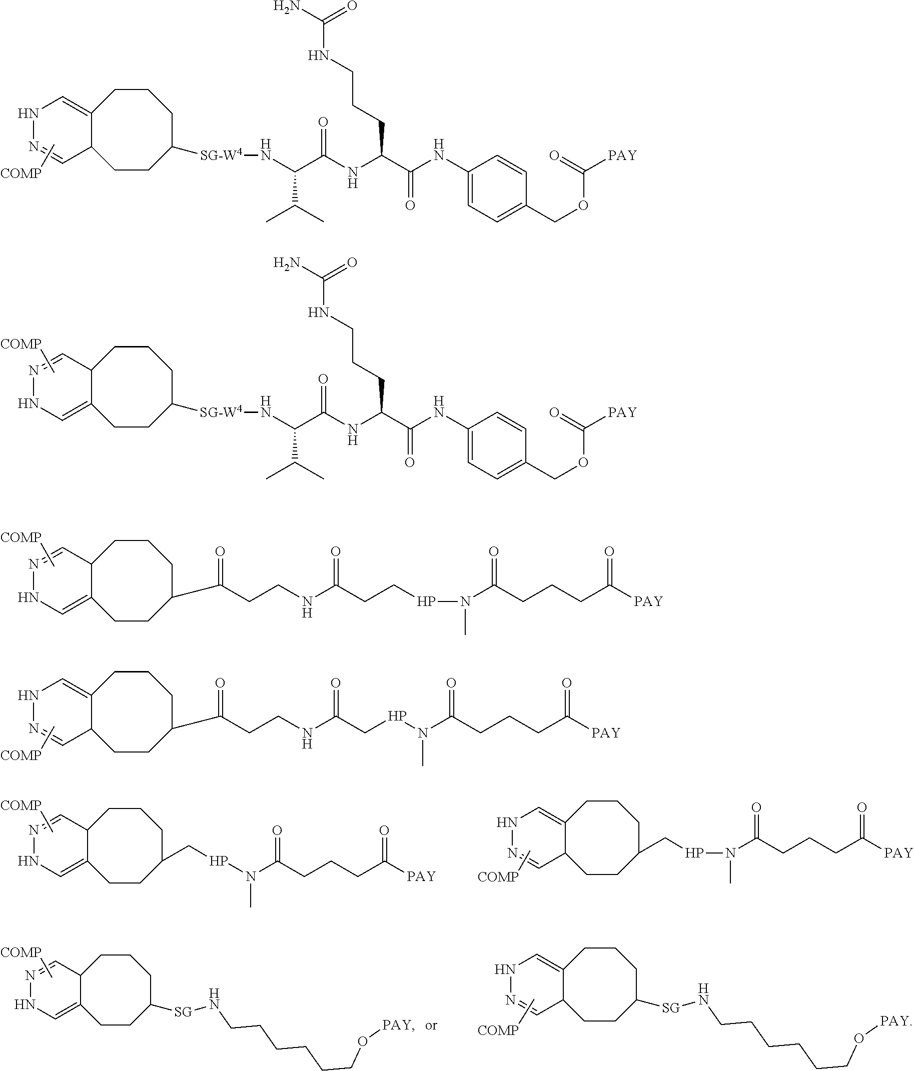

In an embodiment, provided herein is a conjugate according to any of Formulas 101a-104b, where COMP indicates a residue of the anti-Tissue Factor antibody and PAY indicates a payload moiety:

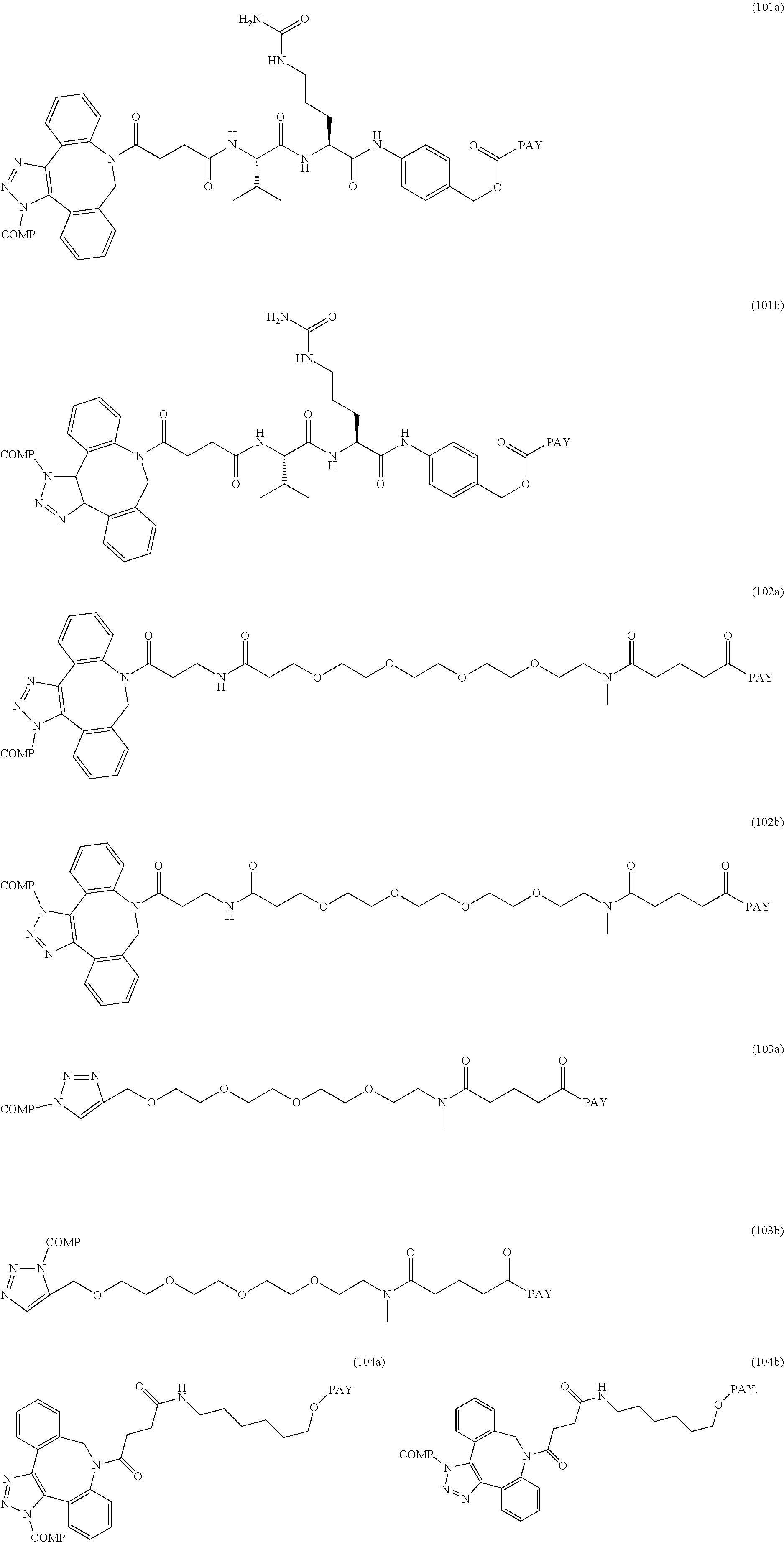

In any of the foregoing embodiments, the conjugate comprises n2 number of PAY moieties, wherein n2 is an integer from 1 to 10. In some embodiments, n2 is 2. In some embodiments, n2 is 3. In some embodiments, n2 is 4. In some embodiments, n2 is 5. In some embodiments, n2 is 6. In some embodiments, n2 is 7. In some embodiments, n2 is 8. In some embodiments, n2 is 9. In some embodiments, n2 is 10.

In some embodiments, provided herein are anti-Tissue Factor conjugates comprising a modified hemiasterlin and linker as described, for example, in PCT Publication No. WO 2016/123582. For example, the conjugate can have a structure comprising any of Formulas 1000-1000b, 1001-1001b, 1002-1002b, and I-XIXb-2, 101-111b, or 1-8b as described in PCT Publication No. WO 2016/123582. Examples of conjugates comprising a modified hemiasterlin and linker are provided below.

In some embodiments, provided is a conjugate of Formula (I)

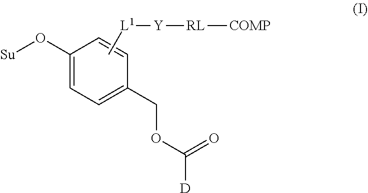

or a pharmaceutically acceptable salt thereof, wherein
COMP is a residue of an anti-Tissue Factor antibody provided herein;
L¹ is —C₁₋₆ alkylene-;
Y is —X¹—C₁₋₆ alkylene-[X¹—C₁₋₆ alkylene]ₙ-[X¹]ₚ—,
—X¹—C₂₋₆ alkenylene-[X¹—C₂₋₆ alkenylene]ₙ-[X¹]ₚ—, —X¹—C₂₋₆ alkynylene-[X¹—C₂₋₆ alkynylene]ₙ—[X¹]ₚ—, wherein at least one alkylene, alkenylene or alkynylene in Y is substituted with one or more substituents selected from R⁵⁰; and
wherein the alkylene, alkenylene, or alkynylene in Y is optionally substituted with one or more substituents selected from R⁵¹;
R⁵⁰ is —C₁₋₆ alkylene-X²—[C₁₋₆ alkylene]ₘ-POLY,
—C₂₋₆ alkenylene-X²—[C₂₋₆ alkenylene]ₘ-POLY, or —C₂₋₆ alkynylene-X²—[C₂₋₆ alkynylene]ₘ-POLY, wherein each alkylene, alkenylene or alkynylene of R⁵⁰ is optionally substituted with one or more substituents selected from halogen, —CN, —NO₂, —OH, —N(R¹⁰)₂, —C(O)N(R¹⁰)₂, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
R⁵¹ is independently selected from halogen, —CN, —NO₂, —OH, —N(R¹⁰)₂, —C(O)N(R¹⁰)₂, —C(O)—, —C(S)—, —C(O)OCH₂C₆H₅,
—NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
X¹ and X² are independently selected from —C(O)— and —N(R¹⁰)C(O)—;
R¹⁰ is independently selected at each occurrence from hydrogen, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
POLY is a water-soluble polymer;
n is an integer selected from zero, one, two, and three;
m is an integer selected from zero and one;
p is an integer selected from zero and one;
Su is a hexose form of a monosaccharide;
D is a drug moiety; and
RL is a reactive group residue.

In some embodiments, provided is a conjugate of Formula (II)

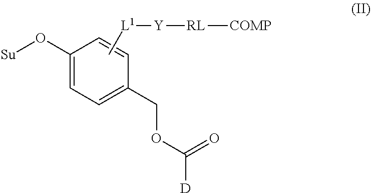

or a pharmaceutically acceptable salt thereof, wherein
COMP is a residue of an anti-Tissue Factor antibody provided herein;
L¹ is —C₁₋₆ alkylene-;
Y is —X¹—C₁₋₆ alkylene-[X¹—C₁₋₆ alkylene]ₙ-X¹—,
—X¹—C₂₋₆ alkenylene-[X¹—C₂₋₆ alkenylene]ₙ—X¹—, —X¹—C₂₋₆ alkynylene-[X¹—C₂₋₆ alkynylene]ₙ—X¹—, wherein at least one alkylene, alkenylene or alkynylene in Y is substituted with one or more substituents selected from R⁵⁰;
R⁵⁰ is —C₁₋₆ alkylene-X²—[C₁₋₆ alkylene]ₘ-POLY,
—C₂₋₆ alkenylene-X²—[C₂₋₆ alkenylene]ₘ-POLY, or —C₂₋₆ alkynylene-X²—[C₂₋₆ alkynylene]ₘ-POLY, wherein each alkylene, alkenylene or alkynylene of R⁵⁰ is optionally substituted with one or more substituents selected from halogen, —CN, —NO₂, —OH, —N(R¹⁰)₂, —C(O)N(R¹⁰)₂,
—C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
X¹ and X² are independently selected from —C(O)— and —N(R¹⁰)C(O)—;
R¹⁰ is independently selected at each occurrence from hydrogen, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl; POLY is a water-soluble polymer;
n is an integer selected from zero, one, two, and three;
m is an integer selected from zero and one;
Su is a hexose form of a monosaccharide;
D is a drug moiety; and
RL is a reactive group residue.

In some embodiments, provided is a conjugate of Formula (IIA)

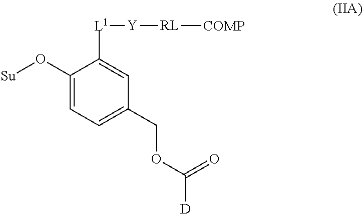

or a pharmaceutically acceptable salt thereof

In some embodiments, the compound of Formula (II) is according to Formula (IIB)

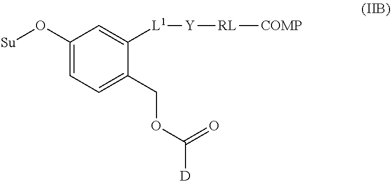

or a pharmaceutically acceptable salt thereof.

In some embodiments of Formula (I), (II), (IIA), or (IIB), L¹ is —C₁₋₃ alkylene-. In some embodiments, L¹ is —CH₂—. In some embodiments of Formula (I), (II), (IIA), or (IIB), L¹ is —CH₂CH₂—. In some embodiments, L¹ is —CH₂CH₂CH₂—.

In some embodiments of Formula (I), including any of the foregoing, p is 0. In some embodiments of Formula (I), including any of the foregoing, p is 1.

In some embodiments of Formula (I), (II), (IIA), or (IIB), including any of the foregoing, Y is —X¹—C₁₋₆ alkylene-[X¹—C₁₋₆ alkylene]ₙ—X¹—, wherein at least one alkylene in Y is substituted with one or more substituents selected from R⁵⁰. In some embodiments of Formula (I), (II), (IIA), or (IIB), including any of the foregoing, Y is —X¹—C₁₋₆ alkylene-[X¹—C₁₋₆ alkylene]ₙ-, wherein at least one alkylene in Y is substituted with one or more substituents selected from R⁵⁰. In some embodiments of Formula (I), (II), (IIA), or (IIB), including any of the foregoing, Y is —X¹—C₂₋₆ alkenylene-[X¹—C₂₋₆ alkenylene]ₙ-X¹— wherein at least one alkenylene in Y is substituted with one or more substituents selected from R⁵⁰. In some embodiments of Formula (I), (II), (IIA), or (IIB), including any of the foregoing, Y is —X¹—C₂₋₆ alkenylene-[X¹—C₂₋₆ alkenylene]ₙ- wherein at least one alkenylene in Y is substituted with one or more substituents selected from R⁵⁰. In some embodiments of Formula (I), (II), (IIA), or (IIB), including any of the foregoing, Y is —X¹—C₂₋₆ alkynylene-[X¹—C₂₋₆ alkynylene]ₙ—X¹— wherein at least one alkynylene in Y is substituted with one or more substituents selected from R⁵⁰. In some embodiments of Formula (I), (II), (IIA), or (IIB), including any of the foregoing, Y is —X¹—C₂₋₆ alkynylene-[X¹—C₂₋₆ alkynylene]ₙ- wherein at least one alkynylene in Y is substituted with one or more substituents selected from R⁵⁰. In some embodiments of Formula (I), (II), (IIA), or (IIB), including any of the foregoing, n is zero. In some embodiments of Formula (I), (II), (IIA), or (IIB), n is one. In some embodiments of Formula (I), (II), (IIA), or (IIB), including any of the foregoing, n is two. In some embodiments, including any of the foregoing, n is three.

In certain embodiments of Formula (I), (II), (IIA), or (IIB), including any of the foregoing, Y is —X¹—C₁₋₄ alkylene-[X¹—C₁₋₄ alkylene]ₙ-X¹—, wherein at least one alkylene in Y is substituted with one or more substituents selected from R⁵⁰. In some embodiments of Formula (I), (II), (IIA), or (IIB), including any of the foregoing, n is zero. In some embodiments of Formula (I), (II), (IIA), or (IIB), including any of the foregoing, n is one. In some embodiments of Formula (I), (II), (IIA), or (IIB), including any of the foregoing, n is two. In some embodiments of Formula (I), (II), (IIA), or (IIB), including any of the foregoing, n is three.

In certain embodiments of Formula (I), (II), (IIA), or (IIB), including any of the foregoing, Y is —X¹—C₁₋₄ alkylene-X¹—C₁₋₄ alkylene-X¹—C₁₋₄ alkylene-X¹—C₁₋₄ alkylene-X¹—, wherein at least one alkylene in Y is substituted with one or more substituents selected from R⁵⁰. In certain embodiments of Formula (I), (II), (IIA), or (IIB), including any of the foregoing, Y is —X¹—C₁₋₄ alkylene-X¹—C₁₋₄ alkylene-X¹—C₁₋₄ alkylene-X¹—, wherein at least one alkylene in Y is substituted with one or more substituents selected from R⁵⁰. In certain embodiments of Formula (I), (II), (IIA), or (IIB), including any of the foregoing, Y is —X¹—C₁₋₄ alkylene-X¹—C₁₋₄ alkylene-X¹—, wherein at least one alkylene in Y is substituted with one or more substituents selected from R⁵⁰.

In certain embodiments of Formula (I), (II), (IIA), or (IIB), including any of the foregoing, R⁵⁰ is —C₁₋₆ alkylene-X²—[C₁₋₆ alkylene]ₘ-POLY, wherein each alkylene of R⁵⁰ is optionally substituted with one or more substituents selected from halogen, —CN, —NO₂, —OH, —N(R¹⁰)₂, —C(O)N(R¹⁰)₂, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl. In some embodiments of Formula (I), (II), (IIA), or (IIB), including any of the foregoing, R⁵⁰ is —C₁₋₄ alkylene-X²—[C₁₋₄ alkylene]ₘ-POLY, wherein each alkylene of R⁵⁰ is optionally substituted with one or more substituents selected from halogen, —CN, —NO₂, —OH, —N(R¹⁰)₂, —C(O)N(R¹⁰)₂, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl. In some embodiments of Formula (I), (II), (IIA), or (IIB), including any of the foregoing, each alkylene of R⁵⁰ is optionally substituted with one or more substituents selected from halogen, —OH, —N(R¹⁰)₂, —C(O)N(R¹⁰)₂, —C(O)—, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl. In some embodiments of Formula (I), (II), (IIA), or (IIB), including any of the foregoing, m is zero. In some embodiments of Formula (I), (II), (IIA), or (IIB), including any of the foregoing, m is one.

In certain embodiments of Formula (I), (II), (IIA), or (IIB), including any of the foregoing, R⁵⁰ is —C₂₋₆ alkenylene-X²—[C₂₋₆ alkenylene]ₘ-POLY, wherein each alkenylene of R⁵⁰ is optionally substituted with one or more substituents selected from halogen, —CN, —NO₂, —OH, —N(R¹⁰)₂, —C(O)N(R¹⁰)₂, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl. In some embodiments of Formula (I), (II), (IIA), or (IIB), including any of the foregoing, m is zero. In some embodiments of Formula (I), (II), (IIA), or (IIB), including any of the foregoing, m is one.

In certain embodiments of Formula (I), (II), (IIA), or (IIB), including any of the foregoing, R⁵⁰ is —C₂₋₆ alkynylene-X²—[C₂₋₆ alkynylene]ₘ-POLY, wherein each alkynylene of R⁵⁰ is optionally substituted with one or more substituents selected from halogen, —CN, —NO₂, —OH, —N(R¹⁰)₂, —C(O)N(R¹⁰)₂, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl. In come embodiments of Formula (I), (II), (IIA), or (IIB), including any of the foregoing, m is zero. In some embodiments of Formula (I), (II), (IIA), or (IIB), including any of the foregoing, m is one.

In certain embodiments of Formula (I), (II), (IIA), or (IIB), including any of the foregoing, POLY is polyethylene glycol (PEG), methoxypolyethylene glycol (mPEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazolines (POZ), poly(N-acryloylmorpholine), polysarcosine, or a combination thereof. In some embodiments of Formula (I), (II), (IIA), or (IIB), including any of the foregoing, POLY is polyethylene glycol (PEG). In some embodiments of Formula (I), (II), (IIA), or (IIB), including any of the foregoing, POLY is methoxypolyethylene glycol (mPEG). In some embodiments of Formula (I), (II), (IIA), or (IIB), including any of the foregoing, POLY is poly(propylene glycol) (PPG). In some embodiments, POLY is copolymers of ethylene glycol and propylene glycol. In some embodiments of Formula (I), (II), (IIA), or (IIB), including any of the foregoing, POLY is poly(oxyethylated polyol). In some embodiments of Formula (I), (II), (IIA), or (IIB), including any of the foregoing, POLY is poly(olefinic alcohol). In some embodiments, POLY is poly(vinylpyrrolidone). In some embodiments of Formula (I), (II), (IIA), or (IIB), including any of the foregoing, POLY is poly(hydroxyalkylmethacrylamide). In some embodiments of Formula (I), (II), (IIA), or (IIB), including any of the foregoing, POLY is poly(hydroxyalkylmethacrylate). In some embodiments of Formula (I), (II), (IIA), or (IIB), including any of the foregoing, POLY is poly(saccharides). In some embodiments of Formula (I), (II), (IIA), or (IIB), including any of the foregoing, POLY is poly(α-hydroxy acid). In some embodiments of Formula (I), (II), (IIA), or (IIB), including any of the foregoing, POLY is poly(vinyl alcohol). In some embodiments of Formula (I), (II), (IIA), or (IIB), including any of the foregoing, POLY is polyphosphazene. In some embodiments of Formula (I), (II), (IIA), or (IIB), including any of the foregoing, POLY is polyoxazolines (POZ). In some embodiments of Formula (I), (II), (IIA), or (IIB), including any of the foregoing, POLY is poly(N-acryloylmorpholine). In some embodiments of Formula (I), (II), (IIA), or (IIB), including any of the foregoing, POLY is polysarcosine. In some embodiments of Formula (I), (II), (IIA), or (IIB), including any of the foregoing, POLY is a nonpeptidic, water-soluble polymer. In certain embodiments of Formula (I), (II), (IIA), or (IIB), including any of the foregoing, POLY includes a polyethylene glycol (PEG) or methoxypolyethylene glycol (mPEG). In certain embodiments of Formula (I), (II), (IIA), or (IIB), including any of the foregoing, POLY is

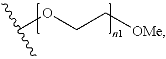

wherein [symbol] represents attachment to the remainder of the compound, and wherein n1 is an integer from one to twenty. In certain embodiments of Formula (I), (II), (IIA), or (IIB), including any of the foregoing, n1 is an integer between five to fifteen. In some embodiments of Formula (I), (II), (IIA), or (IIB), including any of the foregoing, n1 is one. In some embodiments of Formula (I), (II), (IIA), or (IIB), including any of the foregoing, n1 is two. In some embodiments of Formula (I), (II), (IIA), or (IIB), including any of the foregoing, n1 is three. In some embodiments of Formula (I), (II), (IIA), or (IIB), including any of the foregoing, n1 is four. In some embodiments of Formula (II), (IIA), or (IIB), including any of the foregoing, n1 is five. In some embodiments of Formula (I), (II), (IIA), or (IIB), including any of the foregoing, n1 is six. In some embodiment of Formula (I), (II), (IIA), or (IIB)s, n1 is seven. In some embodiments of Formula (I), (II), (IIA), or (IIB), including any of the foregoing, n1 is eight. In some embodiments of Formula (I), (II), (IIA), or (IIB), including any of the foregoing, n1 is nine. In some embodiments of Formula (I), (II), (IIA), or (IIB), including any of the foregoing, n1 is ten. In some embodiments of Formula (I), (II), (IIA), or (IIB), including any of the foregoing, n1 is eleven. In some embodiments of Formula (I), (II), (IIA), or (IIB), including any of the foregoing, n1 is twelve. In some embodiments of Formula (I), (II), (IIA), or (IIB), including any of the foregoing, n1 is thirteen. In some embodiments of Formula (I), (II), (IIA), or (IIB), including any of the foregoing, n1 is fourteen. In some embodiments of Formula (I), (II), (IIA), or (IIB), including any of the foregoing, n1 is fifteen. In some embodiments of Formula (I), (II), (IIA), or (IIB), including any of the foregoing, n1 is sixteen. In some embodiments of Formula (I), (II), (IIA), or (IIB), including any of the foregoing, n1 is seventeen. In some embodiments of Formula (I), (II), (IIA), or (IIB), n1 is eighteen. In some embodiments of Formula (I), (II), (IIA), or (IIB), including any of the foregoing, n1 is nineteen. In some embodiments of Formula (II), (IIA), or (IIB), including any of the foregoing, n1 is twenty. In some embodiments of Formula (I), (II), (IIA), or (IIB), including any of the foregoing, n1 is twenty-one. In some embodiments of Formula (I), (II), (IIA), or (IIB), including any of the foregoing, n1 is twenty-two. In some embodiments of Formula (I), (II), (IIA), or (IIB), including any of the foregoing, n1 is twenty-three. In some embodiments of Formula (I), (II), (IIA), or (IIB), including any of the foregoing, n1 is twenty-four. In some embodiments of Formula (I), (II), (IIA), or (IIB), including any of the foregoing, n1 is twenty-five. In some embodiments of Formula (I), (II), (IIA), or (IIB), including any of the foregoing, n1 is twenty-six. In some embodiments of Formula (I), (II), (IIA), or (IIB), including any of the foregoing, n1 is twenty-seven. In some embodiments of Formula (I), (II), (IIA), or (IIB), including any of the foregoing, n1 is twenty-eight. In some embodiments of Formula (I), (II), (IIA), or (IIB), including any of the foregoing, n1 is twenty-nine. In some embodiments of Formula (I), (II), (IIA), or (IIB), including any of the foregoing, n1 is thirty.

In certain embodiments, RL includes an alkyne, cyclooctyne, a strained alkene, a tetrazine, an amine, methylcyclopropene, a thiol, a para-acetyl-phenylalanine residue, an oxyamine, a maleimide, or an azide. In some embodiments, RL includes an alkyne. In some embodiments, RL includes an cyclooctyne. In some embodiments, RL includes a strained alkene. In some embodiments, RL includes a tetrazine. In some embodiments, RL includes an amine. In some embodiments, RL includes an methylcyclopropene. In some embodiments, RL includes a thiol. In some embodiments, RL includes a para-acetyl-phenylalanine residue. In some embodiments, RL includes an oxyamine. In some embodiments, RL includes a maleimide. In some embodiments, RL includes an azide. In certain embodiments, RL is selected from the group consisting of

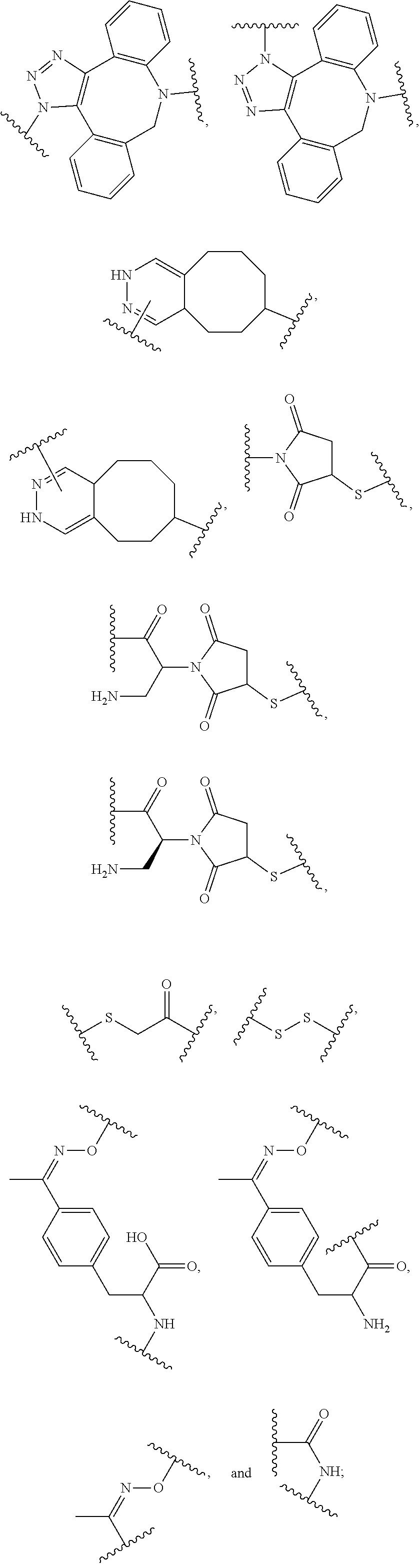

(c) and [symbol] represents attachment to the remainder of the compound. In some embodiments, RL is

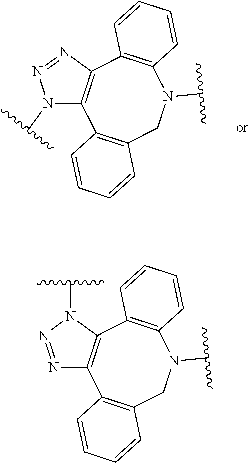 [SMILES: *N1Cc2ccccc2-c2c(nnn2*)-c2ccccc21 ; *N1Cc2ccccc2-c2nnn(*)c2-c2ccccc21]

and [symbol] represents attachment to the remainder of the compound. In one some embodiments, RL is

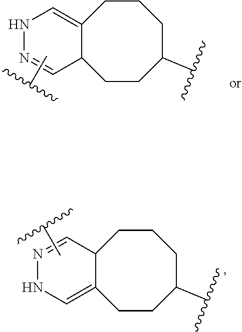

and [symbol] represents attachment to the remainder of the compound. In some embodiments, RL is

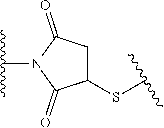 [SMILES: *SC1CC(=O)N(*)C1=O]

and [symbol] represents attachment to the remainder of the compound. In some embodiments, RL is

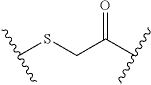 [SMILES: *SCC(*)=O]

and [symbol] represents attachment to the remainder of the compound. In some embodiments, RL is

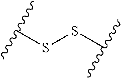 [SMILES: *SS*]

and [symbol] represents attachment to the remainder of the compound. In some embodiments, RL is

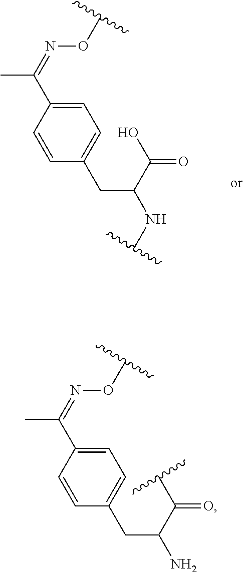

wherein [symbol] represents attachment to the remainder of the compound. In some embodiments, RL is

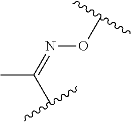 [SMILES: *O/N=C(\*)C]

and [symbol] represents attachment to the remainder of the compound. In some embodiments, RL is

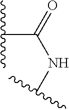 [SMILES: *NC(*)=O]

and [symbol] represents attachment to the remainder of the compound. In some embodiments, RL is

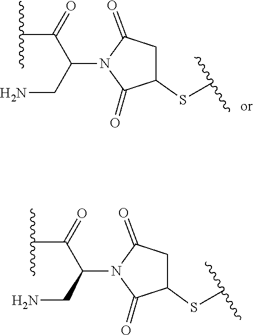 [SMILES: *SC1CC(=O)N(C(CN)C(*)=O)C1=O ; *SC1CC(=O)N([C@@H](CN)C(*)=O)C1=O]

and [symbol] represents attachment to the remainder of the compound. In some embodiments, Su is a sugar moiety. In some embodiments, Su is a hexose form of a monosaccharide. Su may be a glucuronic acid or mannose residue. In certain embodiments, Su is

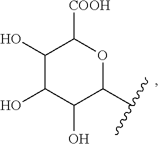

wherein [symbol] represents attachment to the remainder of the compound. In certain embodiments, Su

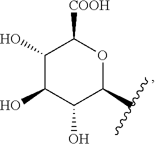

wherein [symbol] represents attachment to the remainder of the compound.

In one aspect, provided herein is a conjugate of Formula (III):

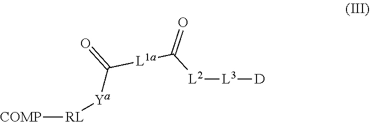

or a pharmaceutically acceptable salt and/or regioisomer thereof;
wherein
L^{1a} is selected from

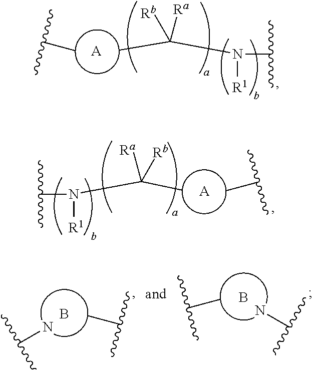

Ring A is an optionally substituted bridged, fused, or spirocyclic bicyclic carbocycle, or an optionally substituted bridged, fused, or spirocyclic bicyclic heterocycle, wherein the carbocycle or the heterocycle of Ring A are optionally substituted with one or more substituents selected from alkyl, alkenyl, alkynyl, cycloalkyl, halogen, alkoxy, —CN, —NO₂, —OH, —N(R²R³)₂, —C(O)—,
—C(O)N(R²R³)₂, —C(O)OR², aminoalkyl, hydroxyalkyl, haloalkyl, aryl, arylalkyl, heteroaryl, and heteroarylalkyl;
Ring B is an optionally substituted N-linked bridged, fused, or spirocyclic bicyclic heterocycle, wherein Ring B is optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
R^{a} and R^{b} are independently selected from hydrogen, alkyl, alkenyl, alkynyl, cycloalkyl, halogen, alkoxy, —CN, —NO₂, —OH, —N(R²R³)₂, —C(O)N(R₂R₃)₂, —C(O)OR², aminoalkyl, hydroxyalkyl, haloalkyl, aryl, arylalkyl, heteroaryl, and heteroarylalkyl;
a is an integer independently selected from 0, 1, 2, 3, 4, 5, and 6;
R¹ is hydrogen or alkyl optionally substituted with one or more substituents selected from cycloalkyl, halogen, alkoxy, —CN, —NO₂, —OH, —N(R²R³)₂, —C(O)N(R₂R₃)₂, —C(O)OR², aryl, and heteroaryl;
R² and R³ are independently selected from hydrogen, alkyl, cycloalkyl, aryl, arylalkyl, heteroaryl, and heteroarylalkyl;
Y^{a} is *—C(O)—(CR^{a}R^{b})_{c}—NH— or *—C(O)—(CR^{a}R^{b})_{c}—, wherein * represents where Y^{a} is bound to RL;
c is an integer selected from 1, 2, 3, 4, 5, or 6;
RL is a reactive group residue;
L² is absent or a linker comprising a hydrophilic polymer residue;
L³ is absent, —C(O)-AA-, —C(O)-AA-Z—(CR^{a}R^{b})ₐ—Z—(CR^{a}R^{b})ₐ—C(O)—, —C(O)—Z—(CR^{a}R^{b})ₐ—C(O)—Z-L⁴-OC(O)—, —Z-AA-, -AA-, —C(O)—, —C(O)-AA-Z—(CRₐR_{b})ₐ—, -AA-C(O)—, —C(O)—(CRₐR_{b})ₐ—Z—(CRₐR_{b})—Z-AA-C(O)—, —C(O)O-L⁴-Z—C(O)—(CRₐR_{b})ₐ—Z—C(O)—, -AA-Z—, or —(CR^{a}R^{b})ₐ—Z-AA-C(O)—;
Z is selected from —NR²— and —O—;
AA is an amino acid residue or a peptide residue;
L⁴ is

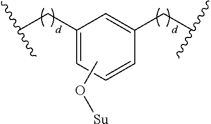

wherein Su is a hexose form of a monosaccharide;
d is an integer independently selected from 1, 2, and 3;
D is a drug moiety;
COMP is a residue of a Tissue Factor antibody; and
[symbol] represents attachment to the remainder of the compound.

In certain embodiments, the compound of Formula (III) is a compound of Formula (IIIA):

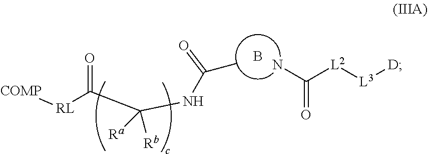

or a pharmaceutically acceptable salt and/or regioisomer thereof;
wherein integer c, RL, R^{a}, R^{b}, Ring B, L², L³, D, and COMP are as defined herein.

In certain embodiments, the compound of Formula (IIIA) is selected from the following:

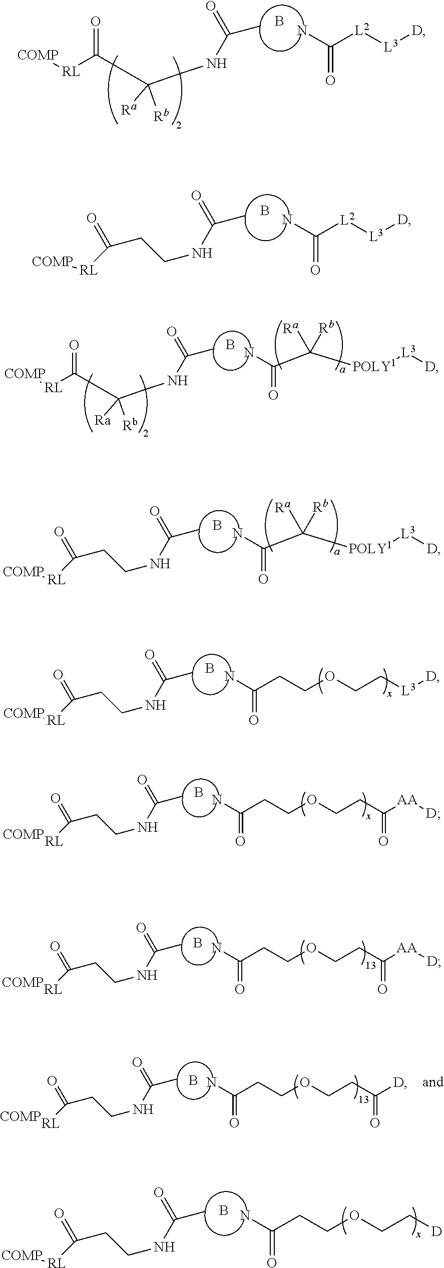

or a pharmaceutically acceptable salt and/or regioisomer thereof.

In certain embodiments, the compound of Formula (III) is a compound of Formula (IIIB):

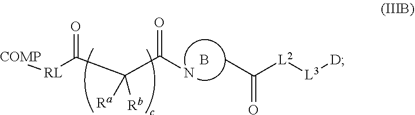

or a pharmaceutically acceptable salt and/or regioisomer thereof;
wherein integer c, RL, R^{a}, R^{b}, Ring B, L², L³, D, and COMP are as defined herein.

In certain embodiments, the compound of Formula (IIIB) is selected from the following:

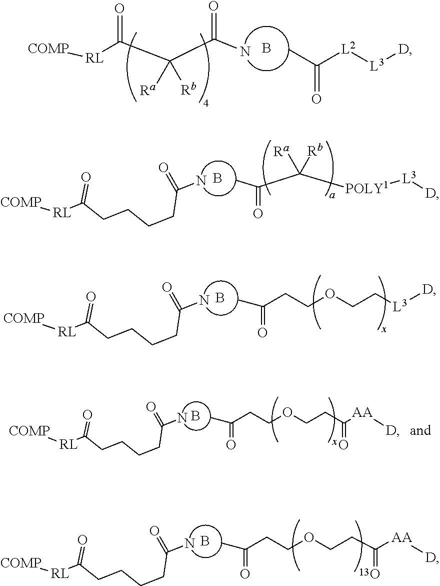

or a pharmaceutically acceptable salt and/or regioisomer thereof.

In certain embodiments, the compound of Formula (III) is a compound of Formula (IIIC):

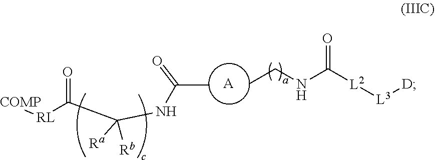

or a pharmaceutically acceptable salt and/or regioisomer thereof;
wherein integer a, integer c, RL, R^{a}, R^{b}, Ring A, L², L³, D, and COMP are as defined herein.

In certain embodiments, the compound of Formula (IIIC) is selected from the

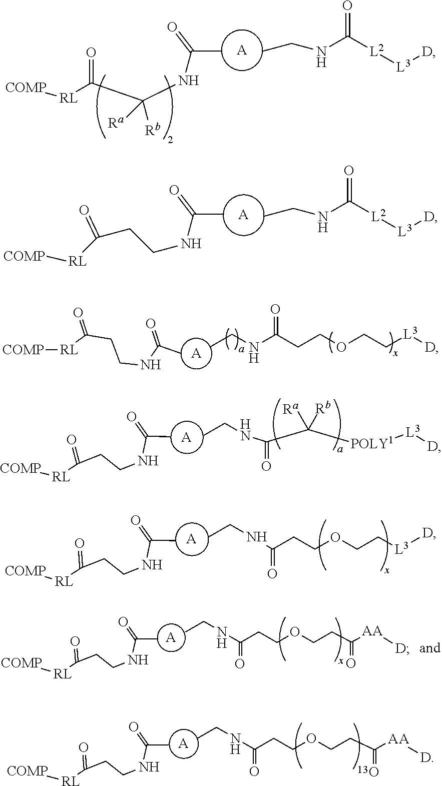

Or a pharmaceutically acceptable salt and/or regioisomer thereof.

In certain embodiments, the compound of Formula (III) is a compound of Formula (IIID):

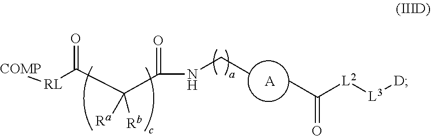

or a pharmaceutically acceptable salt and/or regioisomer thereof;
wherein integer a, integer c, RL, R^{a}, R^{b}, Ring A, L², L³, D, and COMP are as defined herein.

In certain embodiments, the compound of Formula (IIID) is selected from the

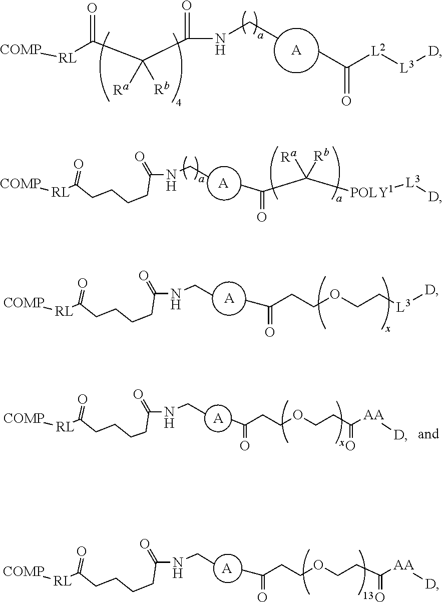

or a pharmaceutically acceptable salt and/or regioisomer thereof.

In certain embodiments, the compound of Formula (III) is a compound of Formula (IIIE):

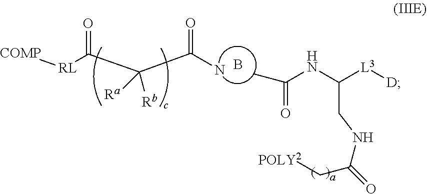

or a pharmaceutically acceptable salt and/or regioisomer thereof;
wherein integer a, integer c, RL, R^{a}, R^{b}, Ring B, L³, POLY², D, and COMP are as defined herein.

In certain embodiments, the compound of Formula (IIIE) is selected from the following:

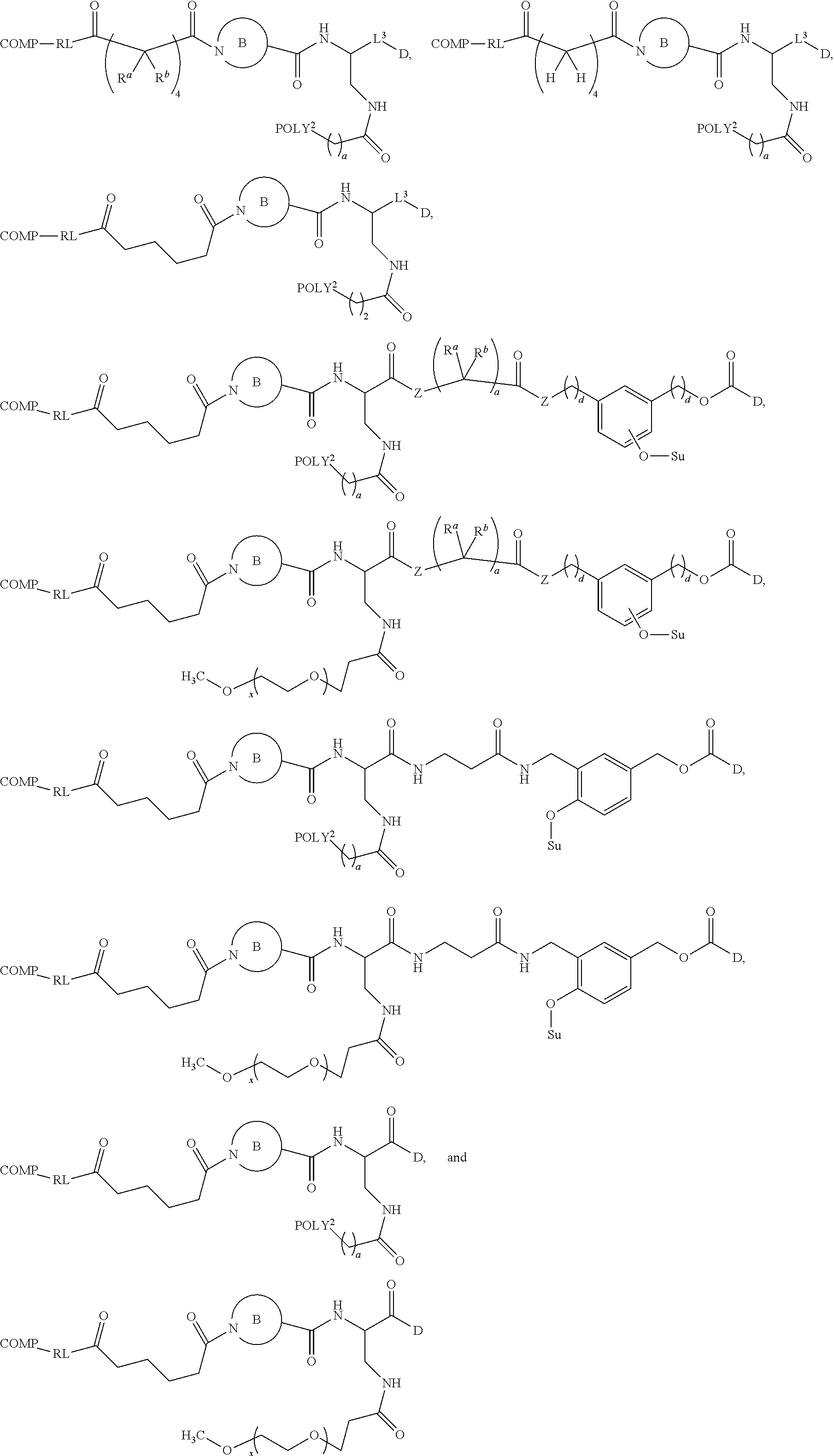

or a pharmaceutically acceptable salt and/or regioisomer thereof.

In certain embodiments, the compound of Formula (III) is a compound of Formula (IIIF):

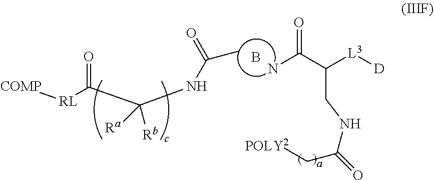

or a pharmaceutically acceptable salt and/or regioisomer thereof;
wherein integer a, integer c, RL, R^{a}, R^{b}, Ring B, L³, POLY², D, and COMP are as defined herein.

In certain embodiments, the compound of Formula (IIIF) is selected from the following:

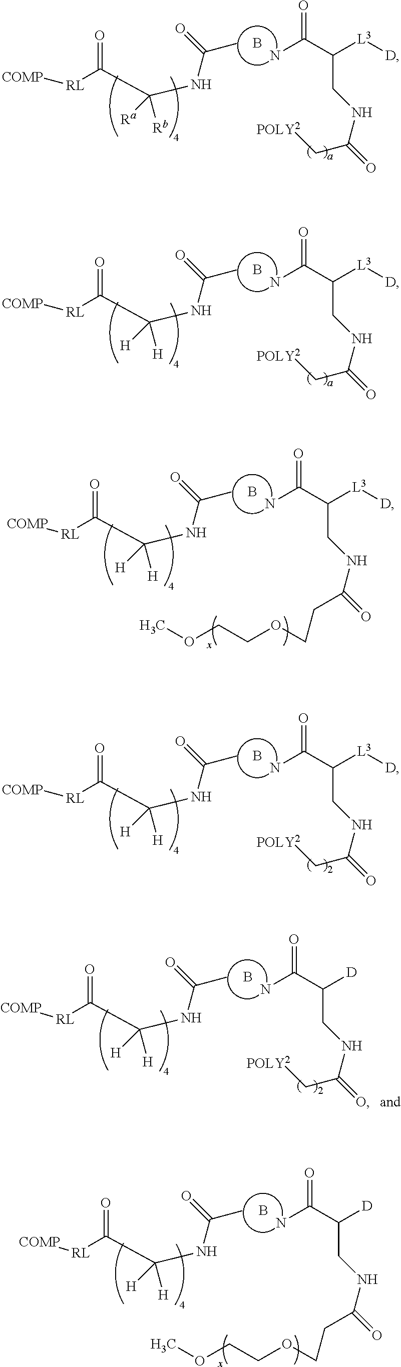

or a pharmaceutically acceptable salt and/or regioisomer thereof.

In certain embodiments, the compound of Formula (III) is a compound of Formula (IIIG):

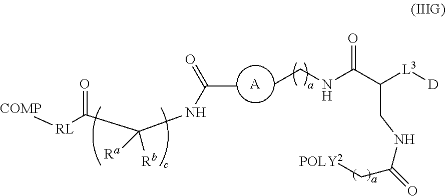

or a pharmaceutically acceptable salt and/or regioisomer thereof;
wherein integer a, integer c, RL, R^{a}, R^{b}, Ring A, L³, POLY², D, and COMP are as defined herein.

In certain embodiments, the compound of Formula (IIIG) is selected from the following:

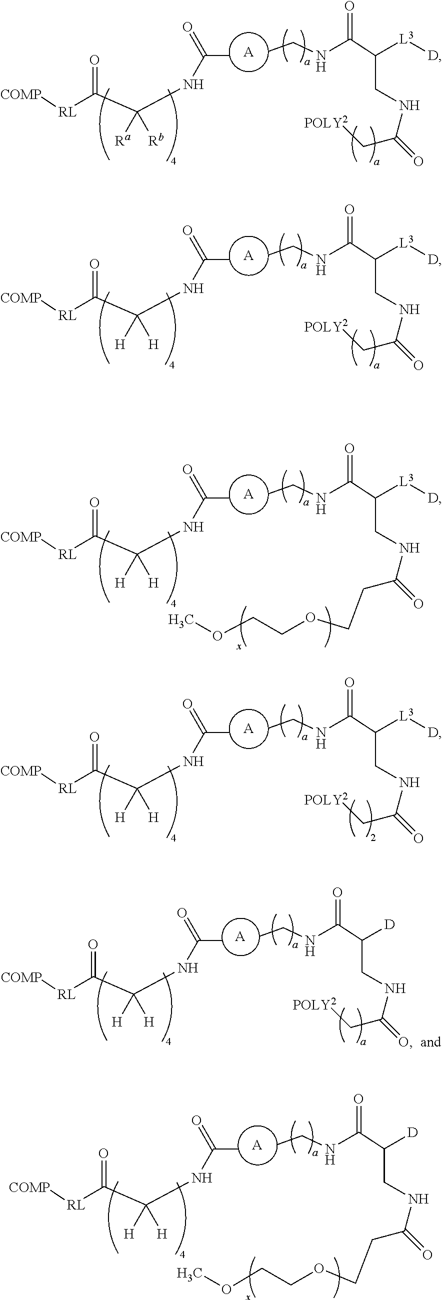

or a pharmaceutically acceptable salt and/or regioisomer thereof.

In certain embodiments, the compound of Formula (III) is a compound of Formula (IIIH):

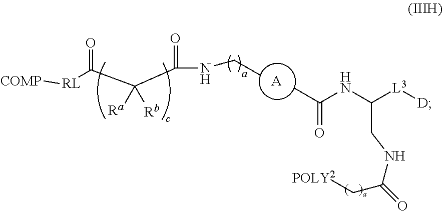

or a pharmaceutically acceptable salt and/or regioisomer thereof;
wherein integer a, integer c, RL, R^{a}, R^{b}, Ring A, L³, POLY², D, and COMP are as defined herein.

In certain embodiments, the compound of Formula (IIIH) is selected from the

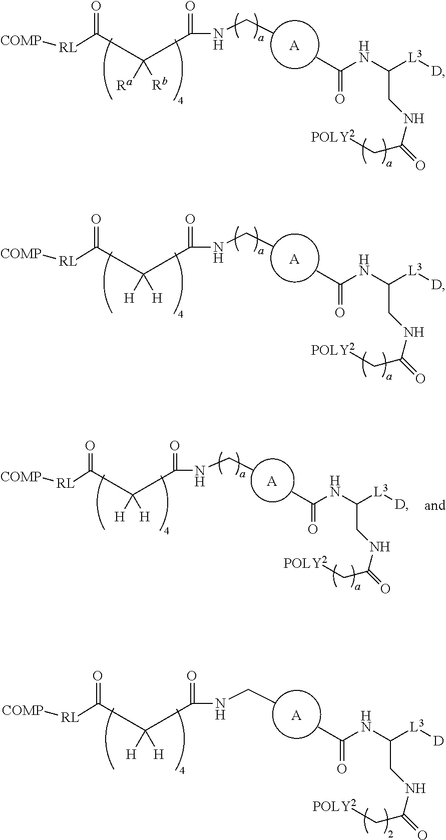

or a pharmaceutically acceptable salt and/or regioisomer thereof.

In one aspect, provided herein is a conjugate of Formula (V):

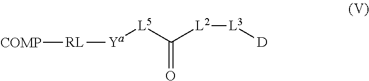

or a pharmaceutically acceptable salt and/or regioisomer thereof;
wherein
L⁵ is a linker comprising an unnatural amino acid; and
RL, COMP, Y^{a}, L², L³, and D are as defined herein.

In certain embodiments, the compound of Formula (V) is a compound of Formula (VA):

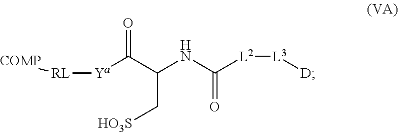

or a pharmaceutically acceptable salt and/or regioisomer thereof;
wherein RL, COMP, Y^{a}, L², L³, and D are as defined herein.

In certain embodiments, the compound of Formula (V) is a compound of Formula (VB):

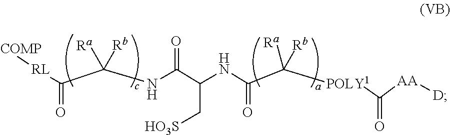

or a pharmaceutically acceptable salt and/or regioisomer thereof;
wherein integer a, integer c, RL, COMP, R^{a}, R^{b}, POLY¹, AA, and D are as defined herein.

In certain embodiments, the compound of Formula (VB) is a compound of the formula:

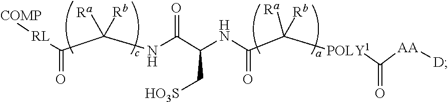

or a pharmaceutically acceptable salt and/or regioisomer thereof.

In certain embodiments of Formula (III)—(VB), including any of the foregoing, L^{1a} is

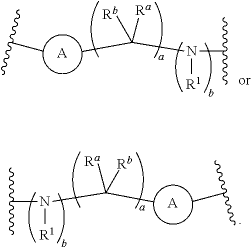

In certain embodiments of Formula (III)—(VB), including any of the foregoing, L^{1a} is

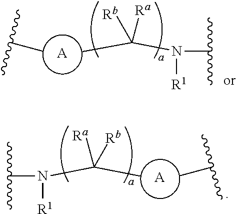

In certain embodiments of Formula (III)—(VB), including any of the foregoing, L^{1a} is

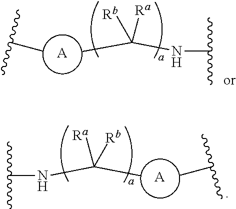

In certain embodiments of Formula (III)—(VB), including any of the foregoing, L^{1a} is

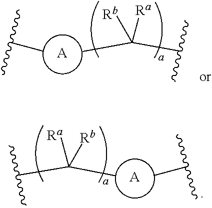

In certain embodiments of Formula (III)—(VB), including any of the foregoing, L^{1a} is

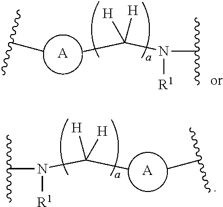

In certain embodiments of Formula (III)—(VB), including any of the foregoing, L^{1a} is

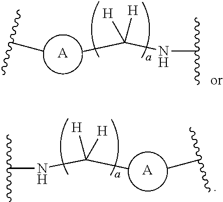

In certain embodiments of Formula (III)—(VB), including any of the foregoing, L^{1a} is

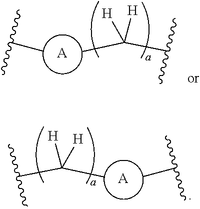

In certain embodiments of Formula (III)—(VB), including any of the foregoing, L^{1a} is

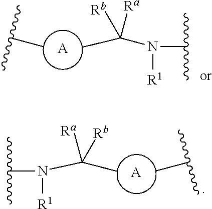

In certain embodiments of Formula (III)—(VB), including any of the foregoing, L^{1a} is

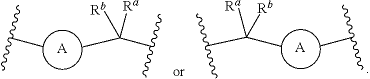

In certain embodiments of Formula (III)—(VB), including any of the foregoing, L^{1a} is

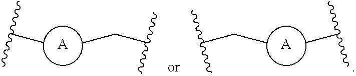

In certain embodiments of Formula (III)—(VB), including any of the foregoing, L^{1a} is

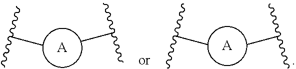

In certain embodiments of Formula (III)—(VB), including any of the foregoing, L^{1a} is

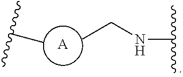

In certain embodiments of Formula (III)—(VB), including any of the foregoing, L^{1a} is

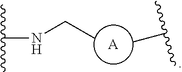

In certain embodiments of Formula (III)—(VB), including any of the foregoing, L^{1a} is

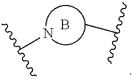

In certain embodiments of Formula (III)—(VB), including any of the foregoing, L^{1a} is

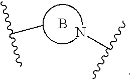

In certain embodiments of Formula (III)—(VB), including any of the foregoing, Ring B of L^{1a} is an optionally substituted 5- to 12-membered N-linked bridged, fused, or spirocyclic bicyclic heterocycle containing 1, 2, or 3 heteroatoms independently selected from N, O, and S including the N to which the ring is attached, wherein Ring B is optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl. In certain embodiments of Formula (III)—(VB), including any of the foregoing, Ring B of L^{1a} is an optionally substituted 5- to 12-membered N-linked spirocyclic bicyclic heterocycle containing 1, 2, or 3 heteroatoms independently selected from N, O, and S including the N to which the ring is attached.

In certain embodiments of Formula (III)—(VB), including any of the foregoing, Ring B of L^{1a} is selected from

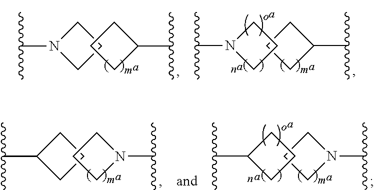

wherein m^{a} is an integer selected from 1, 2, 3, 4, and 5; and
each of n^{a} and o^{a} is an integer independently selected from 1, 2, and 3.

In certain embodiments of Formula (III)—(VB), including any of the foregoing, Ring B of L^{1a} is

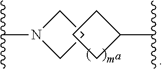

In certain embodiments of Formula (III)—(VB),

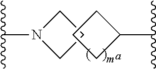

is selected from

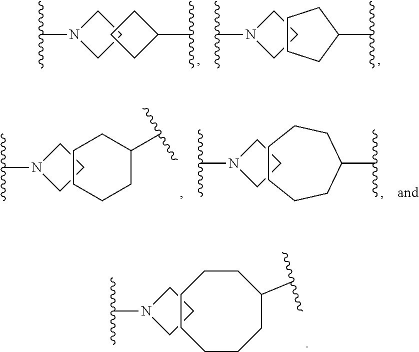

In certain embodiments of Formula (III)—(VB),

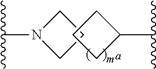

is selected from

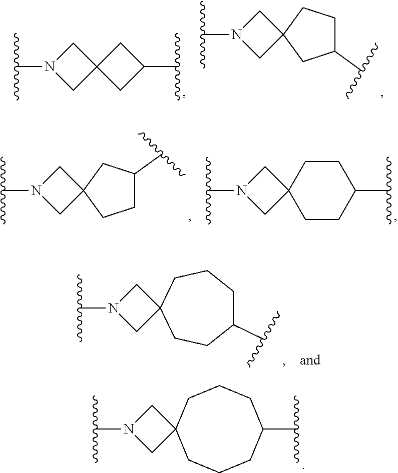

In certain embodiments of Formula (III)—(VB), Ring B of L^{1a} is

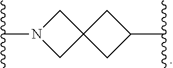 [SMILES: *C1CC2(C1)CN(*)C2]

In certain embodiments, Ring B is

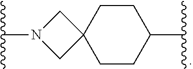 [SMILES: *C1CCC2(CC1)CN(*)C2]

In certain embodiments of Formula (III)—(VB), including any of the foregoing, Ring B of L^{1a} is

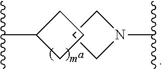

In certain embodiments of Formula (III)—(VB),

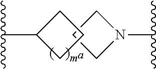

is selected from

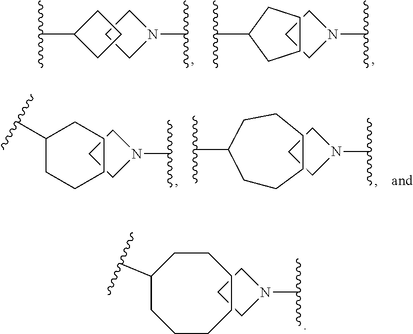

In certain embodiments of Formula (III)—(VB),

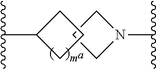

is selected from

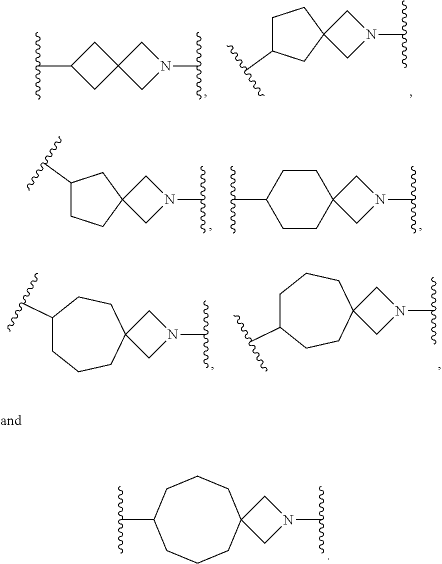

In certain embodiments of Formula (III)—(VB), Ring B of L^{1a} is

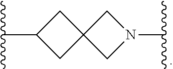 [SMILES: *C1CC2(C1)CN(*)C2]

In certain embodiments of Formula (III)—(VB), Ring B of L^{1a} is

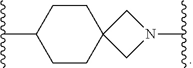 [SMILES: *C1CCC2(CC1)CN(*)C2]

In certain embodiments of Formula (III)—(VB), including any of the foregoing, Ring B of L^{1a} is

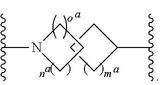

In certain embodiments of Formula (III)—(VB),

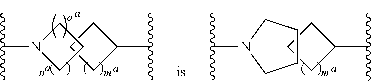

wherein m^{a} is 1, 2, or 3. In certain embodiments of Formula (III)—(VB),

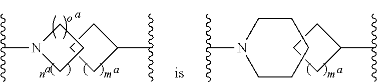

wherein m^{a} is 1, 2, or 3. In certain embodiments of Formula (III)—(VB),

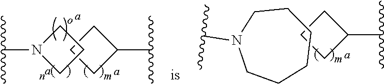

wherein m^{a} is 1, 2, or 3. In certain embodiments of Formula (III)—(VB),

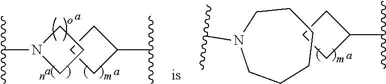

wherein m^{a} is 1, 2, or 3.

In certain embodiments of Formula (III)—(VB),

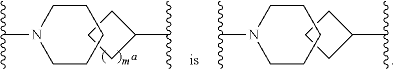

In certain embodiments of Formula (III)—(VB),

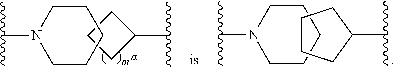

In certain embodiments of Formula (III)—(VB),

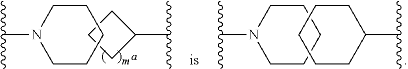

In certain embodiments of Formula (III)—(VB),

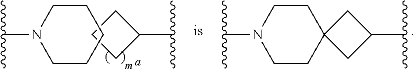

In certain embodiments of Formula (III)—(VB),

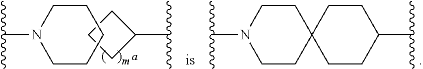

In certain embodiments of Formula (III)—(VB), including any of the foregoing, Ring B of L^{1a} is

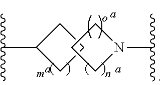

In certain embodiments of Formula (III)—(VB),

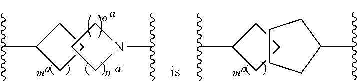

wherein m^{a} is 1, 2, or 3. In certain embodiments of Formula (III)—(VB),

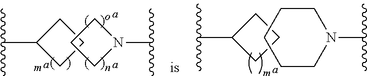

wherein m^{a} is 1, 2, or 3. In certain embodiments of Formula (III)—(VB),

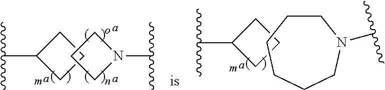

wherein m^{a} is 1, 2, or 3. In certain embodiments of Formula (III)—(VB),

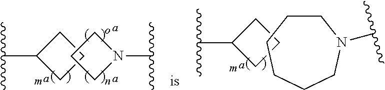

wherein m^{a} is 1, 2, or 3.

In certain embodiments of Formula (III)—(VB),

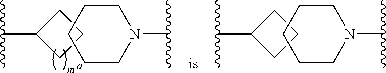

In certain embodiments of Formula (III)—(VB),

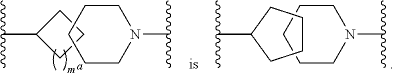

In certain embodiments of Formula (III)—(VB),

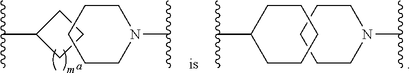

In certain embodiments,

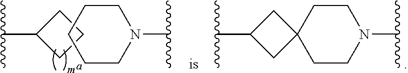

In certain embodiments of Formula (III)—(VB),

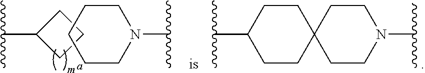

In certain embodiments of Formula (III)—(VB), including any of the foregoing, Ring B of L^{1a} is

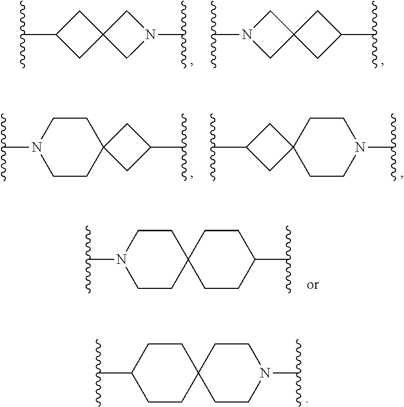

In certain embodiments of Formula (III)—(VB), including any of the foregoing, Ring B of L^{1a} is selected from

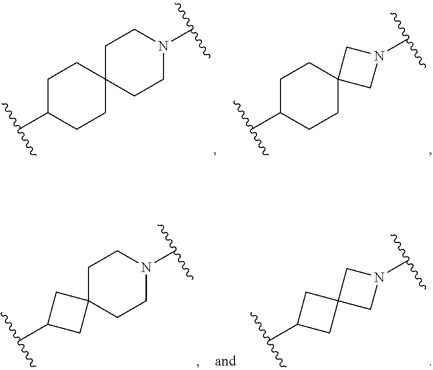 [SMILES: *C1CCC2(CC1)CCN(*)CC2 ; *C1CCC2(CC1)CN(*)C2 ; *C1CC2(CCN(*)CC2)C1 ; *C1CC2(C1)CN(*)C2]

In certain embodiments of Formula (III)—(VB), including any of the foregoing, Ring B of L^{1a} is selected from

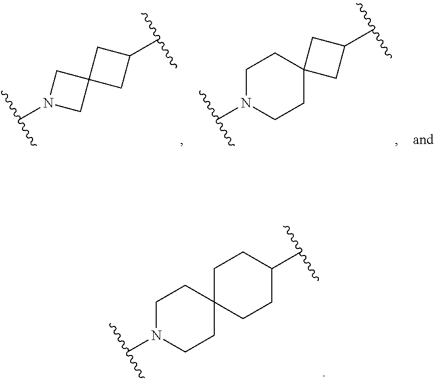 [SMILES: *C1CC2(C1)CN(*)C2 ; *C1CC2(CCN(*)CC2)C1 ; *C1CCC2(CC1)CCN(*)CC2]

In certain embodiments of Formula (III)—(VB), including any of the foregoing, L^{1a} is selected from

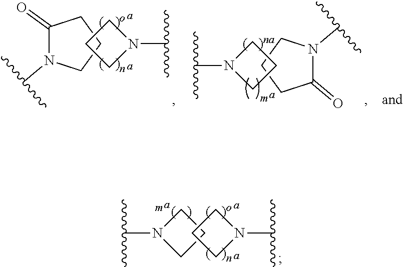

wherein m^{a} is an integer selected from 1, 2, 3, 4, and 5; and
each of n^{a} and o^{a} is an integer independently selected from 1, 2, and 3.

In certain embodiments of Formula (III)—(VB), including any of the foregoing, L^{1a} is selected from

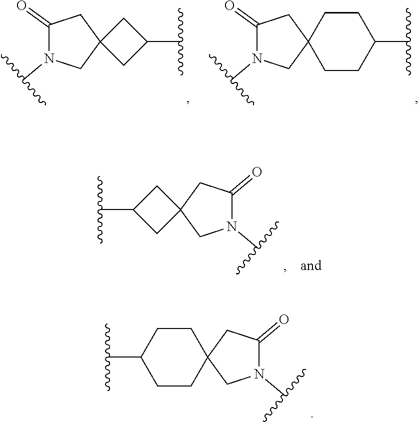 [SMILES: *C1CC2(CC(=O)N(*)C2)C1 ; *C1CCC2(CC1)CC(=O)N(*)C2 ; *C1CC2(CC(=O)N(*)C2)C1 ; *C1CCC2(CC1)CC(=O)N(*)C2]

In certain embodiments of Formula (III)—(VB), including any of the foregoing, Ring B of L^{1a} is selected from

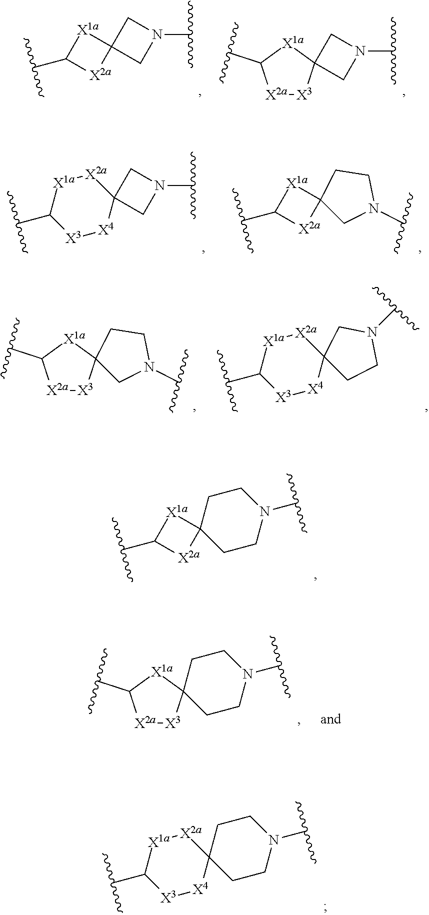

wherein X^{1a}, X^{2a}, X³, and X⁴ are independently selected from —C(R⁴)₂—, —NH—, —O—, and —S— wherein when X^{1a}, X^{2a}, and X³ are present, at least one of X^{1a}—X³ is —C(R⁴)₂— and when X^{1a}, X^{2a}, X³, and X⁴ are present, at least two of X^{1a}—X⁴ are —C(R⁴)₂—; and R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group

In certain embodiments of Formula (III)—(VB), including any of the foregoing, Ring B of L^{1a} is selected from

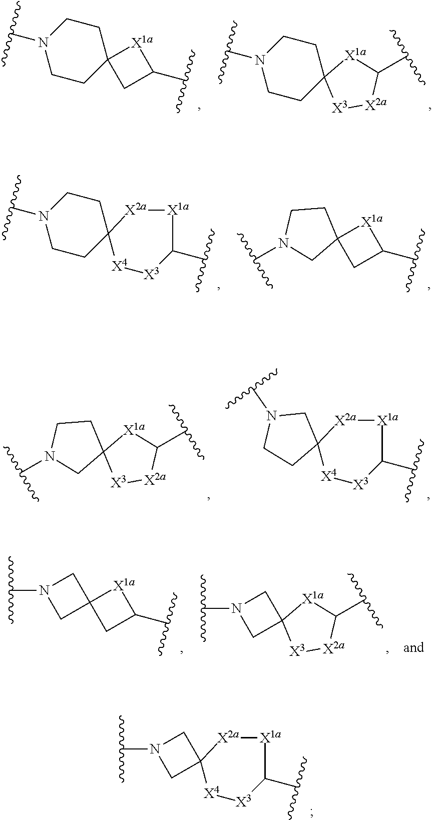

wherein X^{1a}, X^{2a}, X³, and X⁴ are independently selected from —C(R⁴)₂—, —NH—, —O—, and —S— wherein when X^{1a}, X^{2a}, and X³ are present, at least one of X^{1a}—X³ is —C(R⁴)₂— and when X^{1a}, X^{2a}, X³, and X⁴ are present, at least two of X^{1a}—X⁴ are —C(R⁴)₂—; and
R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

In certain embodiments of Formula (III)—(VB), including any of the foregoing, Ring A of L^{1a} is an optionally substituted bridged, fused, or spirocyclic bicyclic carbocycle, wherein the carbocycle or the heterocycle of Ring A are optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl. In certain embodiments, including any of the foregoing, Ring A of L^{1a} is an optionally substituted C₄₋₁₂ bridged, fused, or spirocyclic bicyclic carbocycle. In certain embodiments of Formula (III)—(VB), including any of the foregoing, Ring A of L^{1a} is an optionally substituted C₄₋₁₂ bridged bicyclic carbocycle. In certain embodiments of Formula (III)—(VB), including any of the foregoing, Ring A of L^{1a} is an optionally substituted C₄₋₈ bridged bicyclic carbocycle.

In certain embodiments of Formula (III)—(VB), including any of the foregoing, Ring A of L^{1a} is selected from

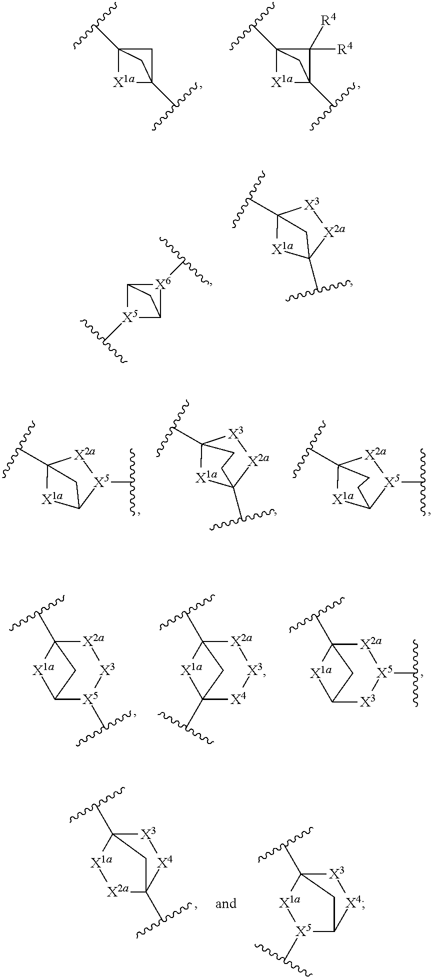

wherein X^{1a}, X^{2a}, X³, and X⁴ are independently selected from —C(R⁴)₂—, —NH—, —O—, and —S— wherein when X^{1a}, X^{2a}, and X³ are present, at least one of X^{1a}—X³ is —C(R⁴)₂— and when X^{1a}, X^{2a}, X³, and X⁴ are present, at least two of X^{1a}—X⁴ are —C(R⁴)₂—;
X⁵ is CR⁴ or N; and
R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

In certain embodiments of Formula (III)—(VB), including any of the foregoing, Ring A of L^{1a} is selected from

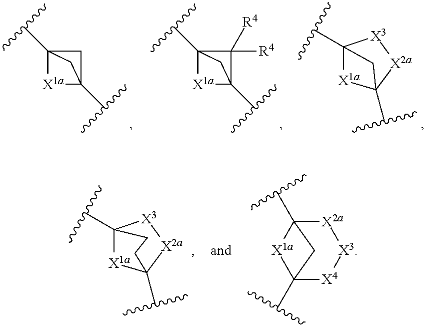

In certain embodiments of Formula (III)—(VB), including any of the foregoing, Ring A of L^{1a} is selected from

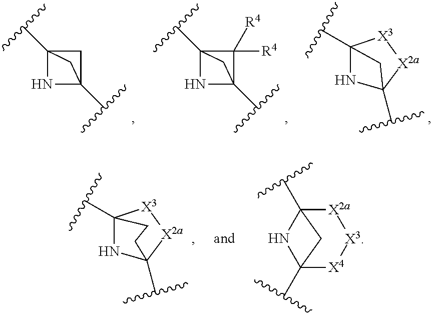

In certain embodiments of Formula (III)—(VB), including any of the foregoing, Ring A of L^{1a} is selected from

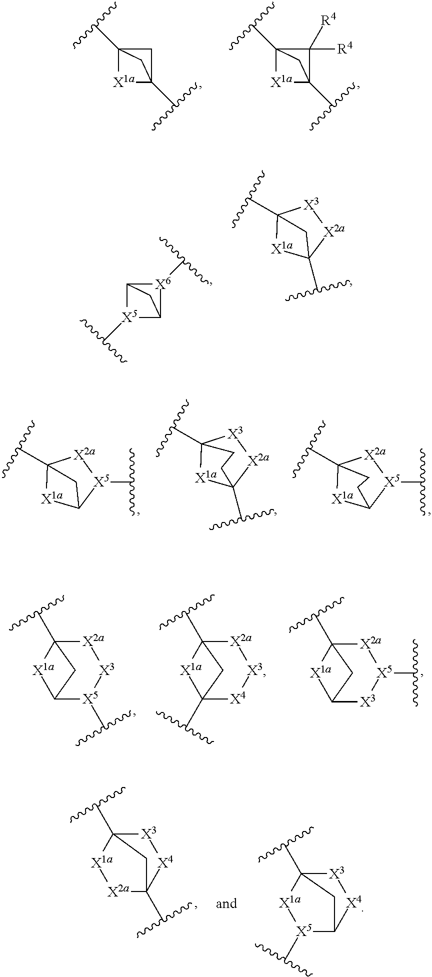

In certain embodiments of Formula (III)—(VB), including any of the foregoing,

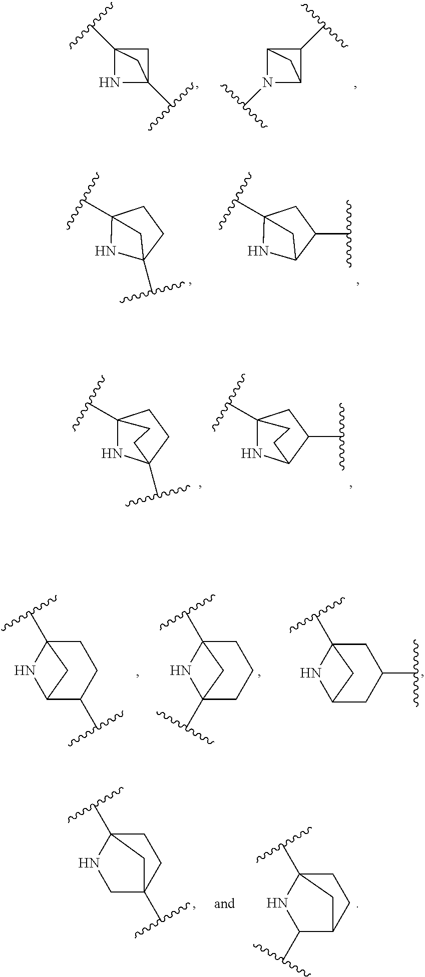

Ring A of L^{1a} is selected from

In certain embodiments of Formula (III)—(VB), including any of the foregoing, Ring A of L^{1a} is

In certain embodiments of Formula (III)—(VB), including any of the foregoing, Ring A of L^{1a} is

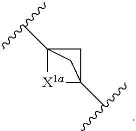

In certain embodiments of Formula (III)—(VB), including any of the foregoing, X^{1a}, X^{2a}, X³, and/or X⁴ is —C(R⁴)₂—.

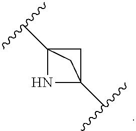 [SMILES: *C12CC(*)(C1)N2]

In certain embodiments of Formula (III)—(VB), including any of the foregoing, X^{1a} and X^{2a} are —C(R⁴)₂—. In certain embodiments of Formula (III)—(VB), including any of the foregoing, X^{1a}, X^{2a}, and X³ are —C(R⁴)₂—. In certain embodiments of Formula (III)—(VB), including any of the foregoing, X^{1a}, X^{2a}, X³, and X⁴ are —C(R⁴)₂—. In certain embodiments of Formula (III)—(VB), including any of the foregoing, X^{1a} is —NH—. In certain embodiments of Formula (III)—(VB), including any of the foregoing, X^{2a} is NH—. In certain embodiments of Formula (III)—(VB), including any of the foregoing, X³ is —NH—. In certain embodiments of Formula (III)—(VB), including any of the foregoing, X⁴ is NH—. In certain embodiments, including any of the foregoing, X^{1a} is —O—. In certain embodiments of Formula (III)—(VB), including any of the foregoing, X^{2a} is —O—. In certain embodiments of Formula (III)—(VB), including any of the foregoing, X³ is —O—. In certain embodiments of Formula (III)—(VB), including any of the foregoing, X⁴ is —O—.

In certain embodiments of Formula (III)—(VB), including any of the foregoing, L^{1a} is

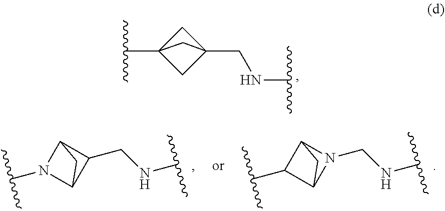 [SMILES: *NCC12CC(*)(C1)C2 ; *NCC1C2CC1N2* ; *NCN1C2CC1C2*]

In certain embodiments, L^{1a} is

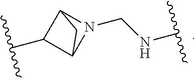 [SMILES: *NCN1C2CC1C2*]

In certain embodiments, including any of the foregoing, a is 0. In certain embodiments, including any of the foregoing, a is 1. In certain embodiments, including any of the foregoing, a is 2. In certain embodiments, including any of the foregoing, a is 3. In certain embodiments, including any of the foregoing, a is 4. In certain embodiments, including any of the foregoing, a is 5. In certain embodiments, including any of the foregoing, a is 6.

In certain embodiments of Formula (III)—(VB), b is 0. In certain embodiments, b is 1.

In certain embodiments of Formula (III)—(VB), b is 0 and a is 0. In certain embodiments of Formula (III)—(VB), b is 0 and a is 1. In certain embodiments of Formula (III)—(VB), b is 0 and a is 2. In certain embodiments of Formula (III)—(VB), b is 0 and a is 3. In certain embodiments of Formula (III)—(VB), b is 0 and a is 4. In certain embodiments of Formula (III)—(VB), b is 0 and a is 5. In certain embodiments of Formula (III)—(VB), b is 0 and a is 6. In certain embodiments of Formula (III)—(VB), b is 1 and a is 1. In certain embodiments of Formula (III)—(VB), b is 1 and a is 2. In certain embodiments of Formula (III)—(VB), b is 1 and a is 3. In certain embodiments of Formula (III)—(VB), b is 1 and a is 4. In certain embodiments of Formula (III)—(VB), b is 1 and a is 5. In certain embodiments of Formula (III)—(VB), b is 1 and a is 6.

In certain embodiments of Formula (III)—(VB), including any of the foregoing, R¹ is hydrogen. In certain embodiments of Formula (III)—(VB), including any of the foregoing, R¹ is unsubstituted alkyl. In certain embodiments of Formula (III)—(VB), including any of the foregoing, R¹ is methyl. In certain embodiments of Formula (III)—(VB), including any of the foregoing, R¹ is alkyl optionally substituted with one or more substituent selected from cycloalkyl, halogen, alkoxy, —CN, —NO₂, and —OH.

In certain embodiments of Formula (III)—(IVB), L⁵ is a linker that comprises at least one amino acid selected from sulfoalanine, hydroxyproline (Hyp), beta-alanine, citrulline (Cit), ornithine (Orn), norleucine (Nle), 3-nitrotyrosine, nitroarginine, pyroglutamic acid (Pyr), naphtylalanine (Nal), 2,4-diaminobutyric acid (DAB), methionine sulfoxide, and methionine sulfone. In certain embodiments of Formula (III)—(IVB), L⁵ is a linker that comprises

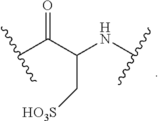 [SMILES: *NC(CS(=O)(=O)O)C(*)=O]

In certain embodiments of Formula (III)—(IVB), L⁵ is a linker that comprises

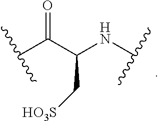 [SMILES: *N[C@@H](CS(=O)(=O)O)C(*)=O]

In certain embodiments of Formula (III)—(IVB), L⁵ is

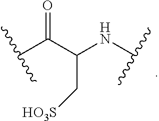 [SMILES: *NC(CS(=O)(=O)O)C(*)=O]

In certain embodiments of Formula (III)—(IVB), L⁵ is

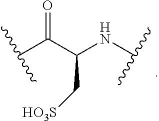 [SMILES: *N[C@@H](CS(=O)(=O)O)C(*)=O]

In certain embodiments, including any of the foregoing, R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl. In certain embodiments, including any of the foregoing, R^{a} is hydrogen and R^{b} is selected from hydrogen, alkyl, alkenyl, alkynyl, cycloalkyl, halogen, alkoxy, —CN, —NO₂, —OH, —NH₂, —C(O)NH₂, —C(O)OH, aminoalkyl, hydroxyalkyl, haloalkyl, aryl, arylalkyl, heteroaryl, and heteroarylalkyl. In certain embodiments, including any of the foregoing, R^{a} is hydrogen and R^{b} is selected from hydrogen, alkyl, halogen, alkoxy, —CN, —NO₂, —OH, —NH₂, —C(O)NH₂, and —C(O)OH. In certain embodiments, including any of the foregoing, R^{a} and R^{b} are both hydrogen.

In certain embodiments of Formula (III)—(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is hydrogen; a is 1; and b is 1. In certain embodiments of Formula (III)—(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is hydrogen; a is 2; and b is 1. In certain embodiments of Formula (III)—(VB), including any of the foregoing, R^{a} is hydrogen; R is selected from hydrogen, alkyl, halogen, alkoxy, —CN, —NO₂, —OH, —NH₂, —C(O)NH₂, and —C(O)OH; R¹ is hydrogen; a is 3; and b is 1.

In certain embodiments of Formula (III)—(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 1; and b is 0. In certain embodiments of Formula (III)—(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 2, and b is 0. In certain embodiments of Formula (III)—(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 3, and b is 0.

In certain embodiments of Formula (III)—(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is methyl; a is 1; and b is 1. In certain embodiments of Formula (III)—(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is methyl; a is 2; and b is 1. In certain embodiments of Formula (III)—(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is methyl; a is 3, and b is 1.

In certain embodiments, including any of the foregoing of Formula (III)—(VB), R^{a} and R^{b} are both hydrogen; R¹ is hydrogen; a is 1, and b is 1. In certain embodiments of Formula (III)—(VB), including any of the foregoing, R^{a} and R^{b} are both hydrogen; R¹ is hydrogen; a is 2, and b is 1. In certain embodiments of Formula (III)—(VB), including any of the foregoing, R^{a} and R^{b} are both hydrogen; R¹ is hydrogen; a is 3, and b is 1. In certain embodiments of Formula (III)—(VB), including any of the foregoing, R^{a} and R^{b} are both hydrogen; R¹ is hydrogen; a is 4, and b is 1. In certain embodiments of Formula (III)—(VB), including any of the foregoing, R^{a} and R^{b} are both hydrogen; R¹ is hydrogen; a is 5, and b is 1. In certain embodiments of Formula (III)—(VB), including any of the foregoing, R^{a} and R^{b} are both hydrogen; R¹ is hydrogen; a is 6, and b is 1.

In certain embodiments of Formula (III)—(VB), including any of the foregoing, R^{a} and R^{b} are both hydrogen; a is 1; and b is 0. In certain embodiments of Formula (III)—(VB), including any of the foregoing, R^{a} and R^{b} are both hydrogen; a is 2; and b is 0. In certain embodiments of Formula (III)—(VB), including any of the foregoing, R^{a} and R^{b} are both hydrogen; a is 3; and b is 0. In certain embodiments of Formula (III)—(VB), including any of the foregoing, R^{a} and R^{b} are both hydrogen; a is 4; and b is 0. In certain embodiments of Formula (III)—(VB), including any of the foregoing, R^{a} and R^{b} are both hydrogen; a is 5; and b is 0. In certain embodiments of Formula (III)—(VB), including any of the foregoing, R^{a} and R^{b} are both hydrogen; a is 6; and b is 0.

In certain embodiments of Formula (III)—(VB), including any of the foregoing, R^{a} and R^{b} are both hydrogen; R¹ is methyl; a is 1; and b is 0. In certain embodiments of Formula (III)—(VB), including any of the foregoing, R^{a} and R^{b} are both hydrogen; R¹ is methyl; a is 2; and b is 0. In certain embodiments of Formula (III)—(VB), including any of the foregoing, R^{a} and R^{b} are both hydrogen; R¹ is methyl; a is 3; and b is 0. In certain embodiments of Formula (III)—(VB), including any of the foregoing, R^{a} and R^{b} are both hydrogen; R¹ is methyl; a is 4; and b is 0. In certain embodiments of Formula (III)—(VB), including any of the foregoing, R^{a} and R^{b} are both hydrogen; R¹ is methyl; a is 5; and b is 0. In certain embodiments of Formula (III)—(VB), including any of the foregoing, R^{a} and R^{b} are both hydrogen; R¹ is methyl; a is 6; and b is 0.

In certain embodiments of Formula (III)—(IVB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 1; and c is 1. In certain embodiments of Formula (III)—(IVB), including any of the foregoing, R^{a} is hydrogen; R is selected from hydrogen, alkyl, halogen, alkoxy, —CN, —NO₂, —OH, —NH₂, —C(O)NH₂, and —C(O)OH; a is 2; and c is 1. In certain embodiments of Formula (III)—(IVB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 3; and c is 1.

In certain embodiments of Formula (III)—(IVB), including any of the foregoing, Y^{a} is *—C(O)—(CR^{a}R^{b})_{c}—NH— wherein * represents where Y^{a} is bound to RL. In certain embodiments of Formula (III)—(IVB), including any of the foregoing, Y^{a} is *—C(O)—(CH₂)_{c}—NH—. In certain embodiments of Formula (III)—(IVB), including any of the foregoing, Y^{a} is *—C(O)—(CH₂)—NH—. In certain embodiments, including any of the foregoing, Y^{a} is *—C(O)—(CH₂)₂—NH—. In certain embodiments of Formula (III)—(IVB), including any of the foregoing, Y^{a} is *—C(O)—(CH₂)₃—NH—. In certain embodiments of Formula (III)—(IVB), including any of the foregoing, Y^{a} is *—C(O)—(CH₂)₄—NH—. In certain embodiments of Formula (III)—(IVB), including any of the foregoing, Y^{a} is *—C(O)—(CH₂)₅—NH—. In certain embodiments of Formula (III)—(IVB), including any of the foregoing, Y^{a} is *—C(O)—(CH₂)₆—NH—.

In certain embodiments of Formula (III)—(IVB), including any of the foregoing, Y^{a} is *—C(O)—(CR^{a}R^{b})—NH—. In certain embodiments of Formula (III)—(IVB), including any of the foregoing, Y^{a} is *—C(O)—(CR^{a}R^{b})₂—NH—. In certain embodiments of Formula (III)—(IVB), including any of the foregoing, Y^{a} is *—C(O)—(CR^{a}R^{b})₃—NH—. In certain embodiments of Formula (III)—(IVB), including any of the foregoing, Y^{a} is *—C(O)—(CR^{a}R^{b})₄—NH—. In certain embodiments of Formula (III)—(IVB), including any of the foregoing, Y^{a} is *—C(O)—(CR^{a}R^{b})₅—NH—. In certain embodiments of Formula (III)—(IVB), including any of the foregoing, Y^{a} is *—C(O)—(CR^{a}R^{b})₆—NH—.

In certain embodiments of Formula (III)—(IVB), including any of the foregoing, Y^{a} is *—C(O)—(CR^{a}R^{b})_{c}—NH— wherein R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl. In certain embodiments of Formula (III)—(IVB), including any of the foregoing, Y^{a} is *—C(O)—(CR^{a}R^{b})₂—NH—, *—C(O)—(CR^{a}R^{b})₃—NH—, or *—C(O)—(CR^{a}R^{b})₄—NH— wherein R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl.

In certain embodiments of Formula (III)—(IVB), including any of the foregoing, Y^{a} is *—C(O)—(CR^{a}R^{b})_{c}— wherein * represents where Y^{a} is bound to RL. In certain embodiments of Formula (III)—(IVB), including any of the foregoing, Y^{a} is *—C(O)—(CH₂)_{c}—. In certain embodiments of Formula (III)—(IVB), including any of the foregoing, Y^{a} is *—C(O)—(CH₂)—. In certain embodiments of Formula (III)—(IVB), including any of the foregoing, Y^{a} is *—C(O)—(CH₂)₂—. In certain embodiments of Formula (III)—(IVB), including any of the foregoing, Y^{a} is *—C(O)—(CH₂)₃—. In certain embodiments of Formula (III)—(IVB), including any of the foregoing, Y^{a} is *—C(O)—(CH₂)₄—. In certain embodiments of Formula (III)—(IVB), including any of the foregoing of Formula (III)—(IVB), Y^{a} is *—C(O)—(CH₂)₅—. In certain embodiments, including any of the foregoing, Y^{a} is *—C(O)—(CH₂)₆—.

In certain embodiments, including any of the foregoing of Formula (III)—(IVB), Y^{a} is *—C(O)—(CR^{a}R^{b})—. In certain embodiments of Formula (III)—(IVB), including any of the foregoing, Y^{a} is *—C(O)—(CR^{a}R^{b})₂—. In certain embodiments of Formula (III)—(IVB), including any of the foregoing, Y^{a} is *—C(O)—(CR^{a}R^{b})₃—. In certain embodiments of Formula (III)—(IVB), including any of the foregoing, Y^{a} is *—C(O)—(CR^{a}R^{b})₄—. In certain embodiments of Formula (III)—(IVB), including any of the foregoing, Y^{a} is *—C(O)—(CR^{a}R^{b})₅—. In certain embodiments of Formula (III)—(IVB), including any of the foregoing, Y^{a} is *—C(O)—(CR^{a}R^{b})₆—.

In certain embodiments of Formula (III)—(IVB), including any of the foregoing, Y^{a} is *—C(O)—(CR^{a}R^{b})_{c}— wherein R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl. In certain embodiments of Formula (III)—(IVB), including any of the foregoing, Y^{a} is *—C(O)—(CR^{a}R^{b})₂—, *—C(O)—(CR^{a}R^{b})₃—, or *—C(O)—(CR^{a}R^{b})₄— wherein R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR₂, aminoalkyl, hydroxyalkyl, haloalkyl, aryl, arylalkyl, heteroaryl, and heteroarylalkyl.

In certain embodiments of Formula (III)—(IVB), including any of the foregoing, Y^{a} is *—C(O)—(CH₂)₂—NH— or *—C(O)—(CH₂)₄—.

In certain embodiments of Formula (III)—(IVB), including any of the foregoing, L² is absent. In certain embodiments of Formula (III)—(IVB), including any of the foregoing, L² is a linker comprising a hydrophilic polymer residue.

In certain embodiments of Formula (III)—(IVB), including any of the foregoing, L² is —(CR^{a}R^{b})ₐ-POLY¹-. In certain embodiments of Formula (III)—(IVB), including any of the foregoing, L² is —CH₂-POLY¹-. In certain embodiments of Formula (III)—(IVB), including any of the foregoing, L² is —(CH₂)₂-POLY¹-. In certain embodiments of Formula (III)—(IVB), including any of the foregoing, L² is —(CH₂)₃-POLY¹-. In certain embodiments of Formula (III)—(IVB), including any of the foregoing, L² is —(CH₂)₄-POLY¹-. In certain embodiments of Formula (III)—(IVB), including any of the foregoing, L² is —(CH₂)₅-POLY¹-. In certain embodiments of Formula (III)—(IVB), including any of the foregoing, L² is —(CH₂)₆-POLY¹-. In certain embodiments of Formula (III)—(IVB), including any of the foregoing, L² is —CR^{a}R^{b}-POLY¹-. In certain embodiments of Formula (III)—(IVB), including any of the foregoing, L² is —(CR^{a}R^{b})₂-POLY¹-. In certain embodiments, including any of the foregoing, L² is —(CR^{a}R^{b})₃-POLY¹-. In certain embodiments of Formula (III)—(IVB), including any of the foregoing, L² is —(CR^{a}R^{b})₄-POLY¹-.

In certain embodiments of Formula (III)—(IVB), including any of the foregoing, L² is -POLY¹-.

In certain embodiments of Formula (III)—(IVB), including any of the foregoing, L² is —(CR^{a}R^{b})ₐ-POLY¹-(CR^{a}R^{b})ₐ—. In certain embodiments of Formula (III)—(IVB), including any of the foregoing, L² is —(CR^{a}R^{b})ₐ-POLY¹-(CR^{a}R^{b})ₐ— wherein a is independently selected from 0, 1, 2, 3, 4, 5, or 6. In certain embodiments of Formula (III)—(IVB), including any of the foregoing, L² is —(CH₂)ₐ-POLY¹-(CH₂)ₐ— wherein a is independently selected from 0, 1, 2, 3, 4, 5, or 6. In certain embodiments of Formula (III)—(IVB), including any of the foregoing, L2 is —(CH₂)ₐ-POLY¹—(CR^{a}R^{b})ₐ— wherein a is selected from 1, 2, 3, 4, 5, or 6. In certain embodiments of Formula (III)—(IVB), including any of the foregoing, L² is —(CR^{a}R^{b})ₐ-POLY¹-(CH₂)ₐ— wherein a is independently selected from 0, 1, 2, 3, 4, 5, or 6.

In certain embodiments of Formula (III)—(IVB), including any of the foregoing, POLY¹ is a diavalent residue of a nonpeptidic, hydrophilic polymer. In certain embodiments of Formula (III)—(IVB), POLY¹ is a diavalent residue of polyethylene glycol (PEG), poly (propylene glycol) (PPG), copolymers of ethylene glycol and propylene glycol, poly (oxyethylated polyol), poly (olefinic alcohol), poly (vinylpyrrolidone), poly (hydroxyalkylmethacrylamide), poly (hydroxyalkylmethacrylate), poly (saccharides), poly (α-hydroxy acid), poly (vinyl alcohol), polyphosphazene, polyoxazolines (POZ), poly (N-acryloylmorpholine), polysarcosine, or a combination thereof. In certain embodiments of Formula (III)—(IVB), including any of the foregoing, POLY¹ is a divalent residue of polyethylene glycol (PEG), poly (propylene glycol) (PPG), or a copolymer of ethylene glycol and propylene glycol.

In certain embodiments of Formula (III)—(IVB), including any of the foregoing, POLY¹ is a diavalent residue of polyethylene glycol (PEG). In certain embodiments of Formula (III)—(IVB), including any of the foregoing, POLY¹ is a diavalent residue of poly (propylene glycol) (PPG). In certain embodiments of Formula (III)—(IVB), including any of the foregoing, POLY¹ is a diavalent residue of copolymers of ethylene glycol and propylene glycol. In certain embodiments of Formula (III)—(IVB), including any of the foregoing, POLY¹ is a diavalent residue of poly (oxyethylated polyol). In certain embodiments of Formula (III)—(IVB), including any of the foregoing, POLY¹ is a diavalent residue of poly (olefinic alcohol). In certain embodiments of Formula (III)—(IVB), including any of the foregoing, POLY¹ is a diavalent residue of poly (vinylpyrrolidone). In certain embodiments of Formula (III)—(IVB), including any of the foregoing, POLY¹ is a diavalent residue of poly (hydroxyalkylmethacrylamide). In certain embodiments of Formula (III)—(IVB), including any of the foregoing, POLY¹ is a diavalent residue of poly (hydroxyalkylmethacrylate). In certain embodiments of Formula (III)—(IVB), including any of the foregoing, POLY¹ is a diavalent residue of poly (saccharides). In certain embodiments of Formula (III)—(IVB), including any of the foregoing, POLY¹ is a diavalent residue of poly (α-hydroxy acid). In certain embodiments of Formula (III)—(IVB), including any of the foregoing, POLY¹ is a diavalent residue of poly (vinyl alcohol). In certain embodiments of Formula (III)—(IVB), including any of the foregoing, POLY¹ is a diavalent residue of polyphosphazene. In certain embodiments of Formula (III)—(IVB), including any of the foregoing, POLY¹ is a diavalent residue of polyoxazolines (POZ). In certain embodiments of Formula (III)—(IVB), including any of the foregoing, POLY¹ is a diavalent residue of poly (N-acryloylmorpholine). In certain embodiments of Formula (III)—(IVB), including any of the foregoing, POLY¹ is a diavalent residue of polysarcosine.

In certain embodiments of Formula (III)—(IVB), including any of the foregoing, POLY¹ is

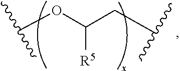

wherein R⁵ is hydrogen or methyl, x is an integer from 1 to 100, inclusive, and [symbol] N represents attachment to the remainder of the compound or conjugate. In certain embodiments of Formula (III)—(IVB), including any of the foregoing, x is an integer between 1 to 25. In certain embodiments of Formula (III)—(IVB), including any of the foregoing, x is an integer between 5 to 15. In some embodiments of Formula (III)—(IVB), including any of the foregoing, x is 1. In some embodiments of Formula (III)—(IVB), including any of the foregoing, x is 2. In some embodiments of Formula (III)—(IVB), including any of the foregoing, x is 3. In some embodiments of Formula (III)—(IVB), including any of the foregoing, x is 4. In some embodiments of Formula (III)—(IVB), including any of the foregoing, x is 5. In some embodiments of Formula (III)—(IVB), including any of the foregoing, x is 6. In some embodiments of Formula (III)—(IVB), including any of the foregoing, x is 7. In some embodiments of Formula (III)—(IVB), including any of the foregoing, x is 8. In some embodiments of Formula (III)—(IVB), including any of the foregoing, x is 9. In some embodiments of Formula (III)—(IVB), including any of the foregoing, x is 10. In some embodiments of Formula (III)—(IVB), including any of the foregoing, x is 11. In some embodiments of Formula (III)—(IVB), including any of the foregoing, x is 12. In some embodiments of Formula (III)—(IVB), including any of the foregoing, x is 13. In some embodiments of Formula (III)—(IVB), including any of the foregoing, x is 14. In some embodiments of Formula (III)—(IVB), including any of the foregoing, x is 15. In some embodiments of Formula (III)—(IVB), including any of the foregoing, x is 16. In some embodiments of Formula (III)—(IVB), including any of the foregoing, x is 17. In some embodiments, including any of the foregoing, x is 18. In some embodiments of Formula (III)—(IVB), including any of the foregoing, x is 19. In some embodiments of Formula (III)—(IVB), including any of the foregoing, x is 20. In certain embodiments of Formula (III)—(IVB), including any of the foregoing, x is an integer between 25 and 50. In certain embodiments of Formula (III)—(IVB), including any of the foregoing, x is an integer between 35 and 45. In certain embodiments of Formula (III)—(IVB), including any of the foregoing, x is an integer between 50 and 75. In certain embodiments of Formula (III)—(IVB), including any of the foregoing, x is an integer between 55 and 65. In certain embodiments of Formula (III)—(IVB), including any of the foregoing, x is an integer between 75 and 100. In certain embodiments of Formula (III)—(IVB), including any of the foregoing, x is an integer between 85 and 95. In certain embodiments of Formula (III)—(IVB), including any of the foregoing, x is an integer in the range of 1 and 25, 20 and 45, 40 and 65, 60 and 85, 70 and 95, or 75 and 100.

In some embodiments of Formula (III)—(IVB), including any of the foregoing, R⁵ is hydrogen. In some embodiments, including any of the foregoing, R⁵ is methyl.

In certain embodiments of Formula (III)—(IVB), including any of the foregoing, L² is —(CR^{a}R^{b})ₐ-POLY¹- wherein POLY¹ is

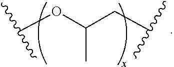

In certain embodiments of Formula (III)—(IVB), including any of the foregoing, L² is —(CR^{a}R^{b})ₐ-POLY¹- wherein POLY¹ is

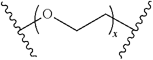

In certain embodiments of Formula (III)—(IVB), including any of the foregoing, L² is —(CH₂)ₐ-POLY¹- wherein POLY¹ is

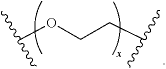

In certain embodiments of Formula (III)—(IVB), including any of the foregoing, L² is —(CH₂)₂-POLY¹- wherein POLY¹ is

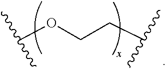

In certain embodiments of Formula (III)—(IVB), including any of the foregoing, L² is —(CR^{a}R^{b})ₐ-POLY¹- wherein POLY¹ is

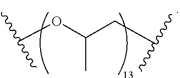

In certain embodiments of Formula (III)—(IVB), including any of the foregoing, L² is —(CR^{a}R^{b})ₐ-POLY¹- wherein POLY¹ is

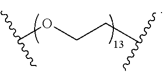

In certain embodiments of Formula (III)—(IVB), including any of the foregoing, L² is —(CH₂)₂-POLY¹- wherein POLY¹ is

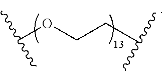

In certain embodiments of Formula (III)—(IVB), including any of the foregoing, L² is —(CH₂)₂-POLY¹- wherein POLY¹ is

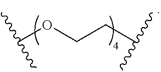

In certain embodiments of Formula (III)—(IVB), including any of the foregoing, L² is —(CH₂)₂-POLY¹- wherein POLY¹ is

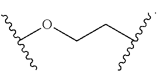 [SMILES: *CCO*]

In certain embodiments of Formula (III)—(IVB), including any of the foregoing, L² is

In certain embodiments, including any of the foregoing, L² is

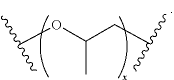

In certain embodiments of Formula (III)—(IVB), including any of the foregoing, L² is

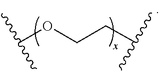

In certain embodiments of Formula (III)—(IVB), including any of the foregoing, L² is

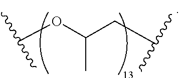

In certain embodiments of Formula (III)—(IVB), including any of the foregoing, L² is

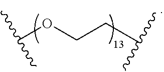

In certain embodiments of Formula (III)—(IVB), including any of the foregoing, L² is —(CR^{a}R^{b})ₐ-POLY¹-(CR^{a}R^{b})ₐ— wherein POLY¹ is

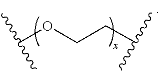

In certain embodiments of Formula (III)—(IVB), including any of the foregoing, L² is —(CR^{a}R^{b})ₐ-POLY¹-(CR^{a}R^{b})ₐ— wherein POLY¹ is

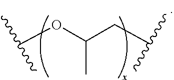

In certain embodiments of Formula (III)—(IVB), including any of the foregoing, L² is —(CR^{a}R^{b})ₐ-POLY¹-(CR^{a}R^{b})ₐ— wherein POLY¹ is

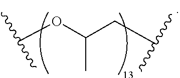

In certain embodiments of Formula (III)—(IVB), including any of the foregoing, L² is —(CR^{a}R^{b})ₐ-POLY¹-(CR^{a}R^{b})ₐ— wherein POLY¹ is

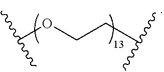

In certain embodiments of Formula (III)—(IVB), including any of the foregoing, L² is selected from

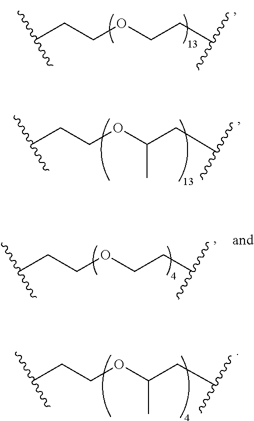

In certain embodiments of Formula (III)—(IVB), including any of the foregoing, L² is

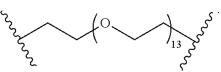

In certain embodiments of Formula (III)—(IVB), including any of the foregoing, L² is

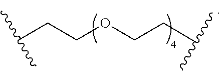

In certain embodiments of Formula (III)—(IVB), including any of the foregoing, L² is

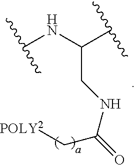

In certain embodiments of Formula (III)—(IVB), including any of the foregoing, L² is

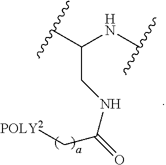

In certain embodiments of Formula (III)—(IVB), including any of the foregoing, L² is

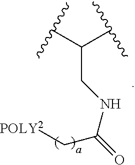

In certain embodiments of Formula (III)—(IVB), including any of the foregoing, L² is

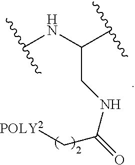

In certain embodiments of Formula (III)—(IVB), including any of the foregoing, L² is

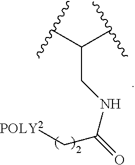

In certain embodiments of Formula (III)—(IVB), including any of the foregoing, POLY² is a residue of a nonpeptidic, hydrophilic polymer. In certain embodiments of Formula (III)—(IVB), POLY² is a residue of polyethylene glycol (PEG), methoxypolyethylene glycol (mPEG), poly (propylene glycol) (PPG), copolymers of ethylene glycol and propylene glycol, poly (oxyethylated polyol), poly (olefinic alcohol), poly (vinylpyrrolidone), poly (hydroxyalkylmethacrylamide), poly (hydroxyalkylmethacrylate), poly (saccharides), poly (α-hydroxy acid), poly (vinyl alcohol), polyphosphazene, polyoxazolines (POZ), poly (N-acryloylmorpholine), polysarcosine, or a combination thereof. In certain embodiments of Formula (III)—(IVB), including any of the foregoing, POLY² is a residue of polyethylene glycol (PEG), methoxypolyethylene glycol (mPEG), poly (propylene glycol) (PPG), or a copolymer of ethylene glycol and propylene glycol. In certain embodiments of Formula (III)—(IVB), including any of the foregoing, POLY² is a residue of methoxypolyethylene glycol (mPEG).

In certain embodiments of Formula (III)—(IVB), including any of the foregoing, POLY² is a residue of polyethylene glycol (PEG). In certain embodiments of Formula (III)—(IVB), including any of the foregoing, POLY² is a residue of poly (propylene glycol) (PPG). In certain embodiments, including any of the foregoing, POLY² is a residue of copolymers of ethylene glycol and propylene glycol. In certain embodiments of Formula (III)—(IVB), including any of the foregoing, POLY² is a residue of poly (oxyethylated polyol). In certain embodiments of Formula (III)—(IVB), including any of the foregoing, POLY² is a residue of poly (olefinic alcohol). In certain embodiments of Formula (III)—(IVB), including any of the foregoing, POLY² is a residue of poly (vinylpyrrolidone). In certain embodiments of Formula (III)—(IVB), including any of the foregoing, POLY² is a residue of poly (hydroxyalkylmethacrylamide). In certain embodiments of Formula (III)—(IVB), including any of the foregoing, POLY² is a residue of poly (hydroxyalkylmethacrylate). In certain embodiments of Formula (III)—(IVB), including any of the foregoing, POLY² is a residue of poly (saccharides). In certain embodiments of Formula (III)—(IVB), including any of the foregoing, POLY² is a residue of poly (α-hydroxy acid). In certain embodiments of Formula (III)—(IVB), including any of the foregoing, POLY² is a residue of poly (vinyl alcohol). In certain embodiments of Formula (III)—(IVB), including any of the foregoing, POLY² is a residue of polyphosphazene. In certain embodiments of Formula (III)—(IVB), including any of the foregoing, POLY² is a residue of polyoxazolines (POZ). In certain embodiments of Formula (III)—(IVB), including any of the foregoing, POLY² is a residue of poly (N-acryloylmorpholine). In certain embodiments of Formula (III)—(IVB), including any of the foregoing, POLY² is a residue of polysarcosine.

In certain embodiments of Formula (III)—(IVB), including any of the foregoing, POLY² is

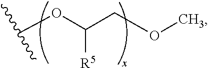

wherein R⁵ is hydrogen or methyl, x is an integer from 1 to 100, inclusive, and [symbol] represents attachment to the remainder of the compound or conjugate. In certain embodiments of Formula (III)—(IVB), including any of the foregoing, x is an integer between 1 to 25. In certain embodiments of Formula (III)—(IVB), including any of the foregoing, x is an integer between 5 to 15. In some embodiments of Formula (III)—(IVB), including any of the foregoing, x is 1. In some embodiments of Formula (III)—(IVB), including any of the foregoing, x is 2. In some embodiments of Formula (III)—(IVB), including any of the foregoing, x is 3. In some embodiments of Formula (III)—(IVB), including any of the foregoing, x is 4. In some embodiments of Formula (III)—(IVB), including any of the foregoing, x is 5. In some embodiments of Formula (III)—(IVB), including any of the foregoing, x is 6. In some embodiments, including any of the foregoing, x is 7. In some embodiments of Formula (III)—(IVB), including any of the foregoing, x is 8. In some embodiments of Formula (III)—(IVB), including any of the foregoing, x is 9. In some embodiments of Formula (III)—(IVB), including any of the foregoing, x is 10. In some embodiments of Formula (III)—(IVB), including any of the foregoing, x is 11. In some embodiments of Formula (III)—(IVB), including any of the foregoing, x is 12. In some embodiments of Formula (III)—(IVB), including any of the foregoing, x is 13. In some embodiments of Formula (III)—(IVB), including any of the foregoing, x is 14. In some embodiments of Formula (III)—(IVB), including any of the foregoing, x is 15. In some embodiments of Formula (III)—(IVB), including any of the foregoing, x is 16. In some embodiments of Formula (III)—(IVB), including any of the foregoing of Formula (III)—(IVB), x is 17. In some embodiments, including any of the foregoing of Formula (III)—(IVB), x is 18. In some embodiments of Formula (III)—(IVB), including any of the foregoing, x is 19. In some embodiments of Formula (III)—(IVB), including any of the foregoing, x is 20. In certain embodiments of Formula (III)—(IVB), including any of the foregoing, x is an integer between 25 and 50. In certain embodiments of Formula (III)—(IVB), including any of the foregoing, x is an integer between 35 and 45. In certain embodiments of Formula (III)—(IVB), including any of the foregoing, x is an integer between 50 and 75. In certain embodiments of Formula (III)—(IVB), including any of the foregoing, x is an integer between 55 and 65. In certain embodiments of Formula (III)—(IVB), including any of the foregoing, x is an integer between 75 and 100. In certain embodiments of Formula (III)—(IVB), including any of the foregoing, x is an integer between 85 and 95. In certain embodiments of Formula (III)—(IVB), including any of the foregoing, x is an integer in the range of 1 and 25, 20 and 45, 40 and 65, 60 and 85, 70 and 95, or 75 and 100.

In some embodiments of Formula (III)—(IVB), including any of the foregoing, R⁵ is hydrogen. In some embodiments of Formula (III)—(IVB), including any of the foregoing, R⁵ is methyl.

In certain embodiments of Formula (III)—(IVB), including any of the foregoing, L² is selected from the group consisting of

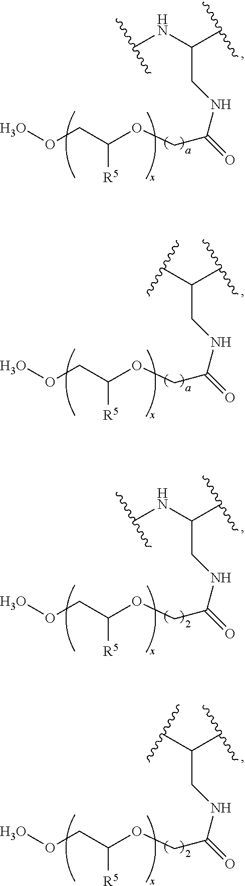

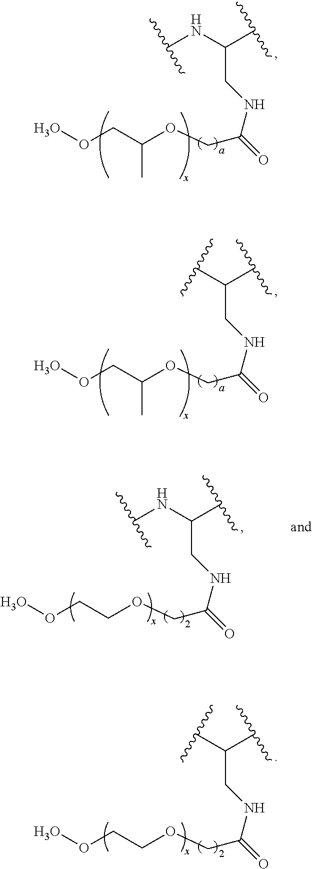

In certain embodiments of Formula (III)—(IVB), including any of the foregoing, L² is selected from the group consisting of

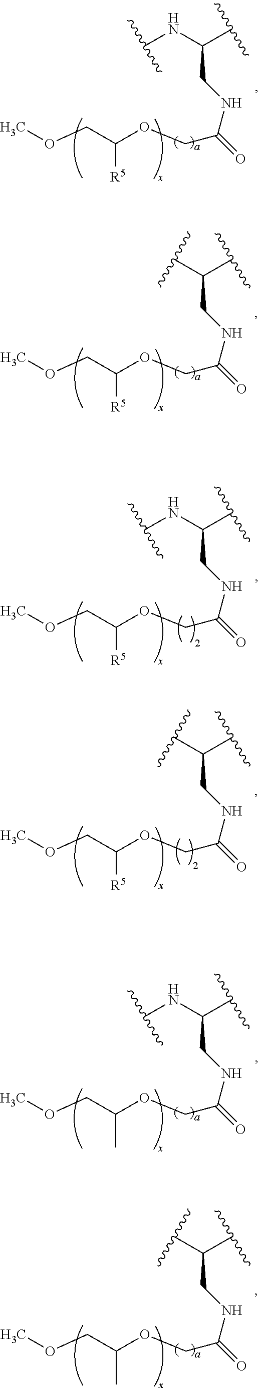

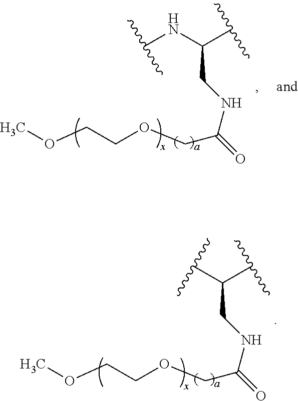

In certain embodiments of Formula (III)—(IVB), including any of the foregoing, L² is

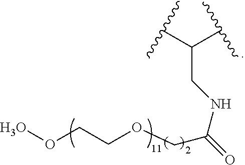

In certain embodiments of Formula (III)—(IVB), including any of the foregoing, L² is

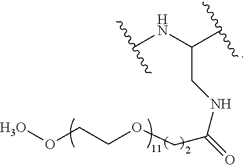

In certain embodiments of Formula (III)—(IVB), including any of the foregoing, L² is

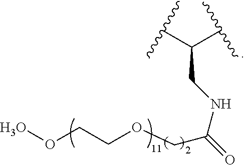

In certain embodiments of Formula (III)—(IVB), including any of the foregoing, L² is

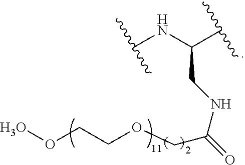

In certain embodiments of Formula (III)—(IVB), including any of the foregoing, L³ is —C(O)-AA-.

In certain embodiments of Formula (III)—(IVB), including any of the foregoing, L³ is —C(O)-AA-Z—(CR^{a}R^{b})ₐ—Z—(CR^{a}R^{b})ₐ—C(O)—. In certain embodiments of Formula (III)—(IVB), including any of the foregoing, L³ is —C(O)-AA-NR²—(CR^{a}R^{b})ₐ—NR²—(CR^{a}R^{b})ₐ—C(O)—. In certain embodiments of Formula (III)—(IVB), including any of the foregoing, L³ is —C(O)-AA-NH—(CR^{a}R^{b})ₐ—NH—(CR^{a}R^{b})ₐ—C(O)—. In certain embodiments of Formula (III)—(IVB), including any of the foregoing, L³ is —C(O)-AA-NH—(CH₂)ₐ—NH—(CH₂)ₐ—C(O)—. In certain embodiments of Formula (III)—(IVB), including any of the foregoing, L³ is —C(O)-AA-NH—(CH₂)ₐ—NH—(CH₂)ₐ—C(O)— wherein a is selected from 1, 2, and 3. In certain embodiments, including any of the foregoing, L³ is —C(O)-AA-NH—CH₂—NH—CH₂—C(O)—

In certain embodiments of Formula (III)—(IVB), including any of the foregoing, L³ is —C(O)-AA-Z—(CR^{a}R^{b})ₐ. In certain embodiments, including any of the foregoing, L³ is —C(O)-AA-NR²—(CH₂)ₐ. In certain embodiments of Formula (III)—(IVB), including any of the foregoing, L³ is —C(O)-AA-NH—(CH₂)₂.

In certain embodiments of Formula (III)—(IVB), including any of the foregoing, L³ is -AA-. In certain embodiments, including any of the foregoing, L³ is

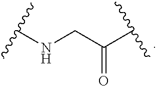 [SMILES: *NCC(*)=O]

In certain embodiments of Formula (III)—(IVB), including any of the foregoing, -AA- is an amino acid residue. In certain embodiments of Formula (III)—(IVB), including any of the foregoing, -AA- is a peptide residue. In certain embodiments of Formula (III)—(IVB), including any of the foregoing, -AA- is a dipeptide residue, a tripeptide residue, a tetrapeptide residue, or a pentapeptide residue. In certain embodiments of Formula (III)—(IVB), including any of the foregoing, -AA- comprises at least one amino acid residue selected from alanine, glycine, valine, and asparagine. In certain embodiments of Formula (III)—(IVB), including any of the foregoing, -AA- comprises at least one amino acid residue selected from alanine and glycine. In certain embodiments of Formula (III)—(IVB), including any of the foregoing, -AA- is selected from the group consisting of

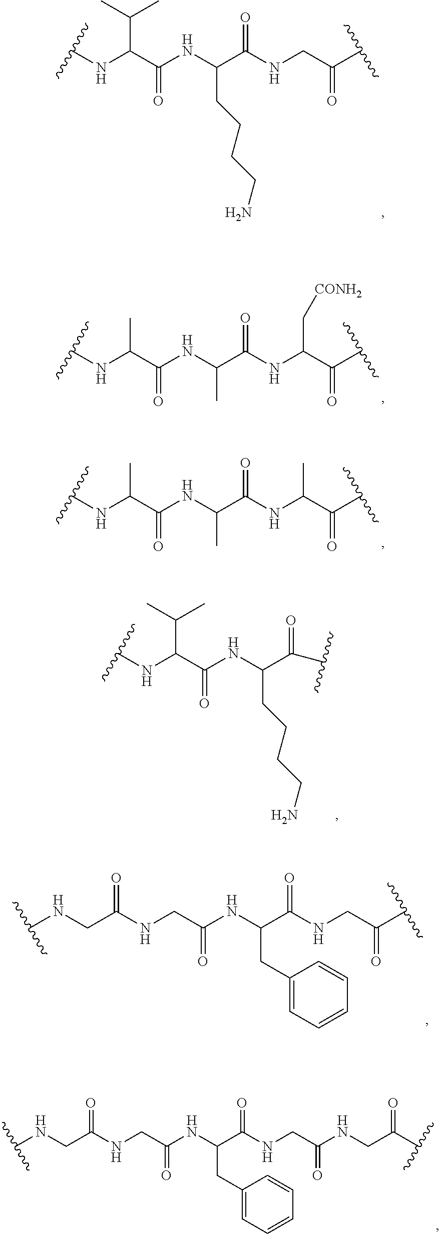 [SMILES: *NC(C(=O)NC(CCCCN)C(=O)NCC(*)=O)C(C)C ; *NC(C)C(=O)NC(C)C(=O)NC(CC(N)=O)C(*)=O ; *NC(C)C(=O)NC(C)C(=O)NC(C)C(*)=O ; *NC(C(=O)NC(CCCCN)C(*)=O)C(C)C ; *NCC(=O)NCC(=O)NC(Cc1ccccc1)C(=O)NCC(*)=O ; *NCC(=O)NCC(=O)NC(Cc1ccccc1)C(=O)NCC(=O)NCC(*)=O]

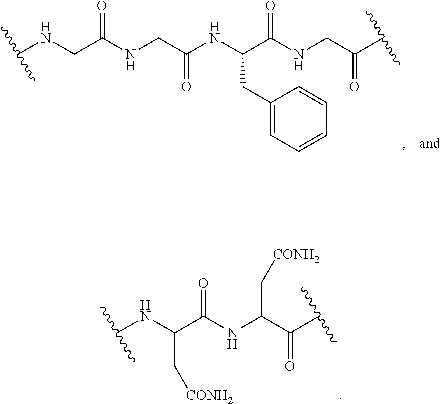 [SMILES: *NCC(=O)NCC(=O)N[C@@H](Cc1ccccc1)C(=O)NCC(*)=O ; *NC(CC(N)=O)C(=O)NC(CC(N)=O)C(*)=O]

In certain embodiments of Formula (III)—(IVB), including any of the foregoing, -AA- is selected from the group consisting of

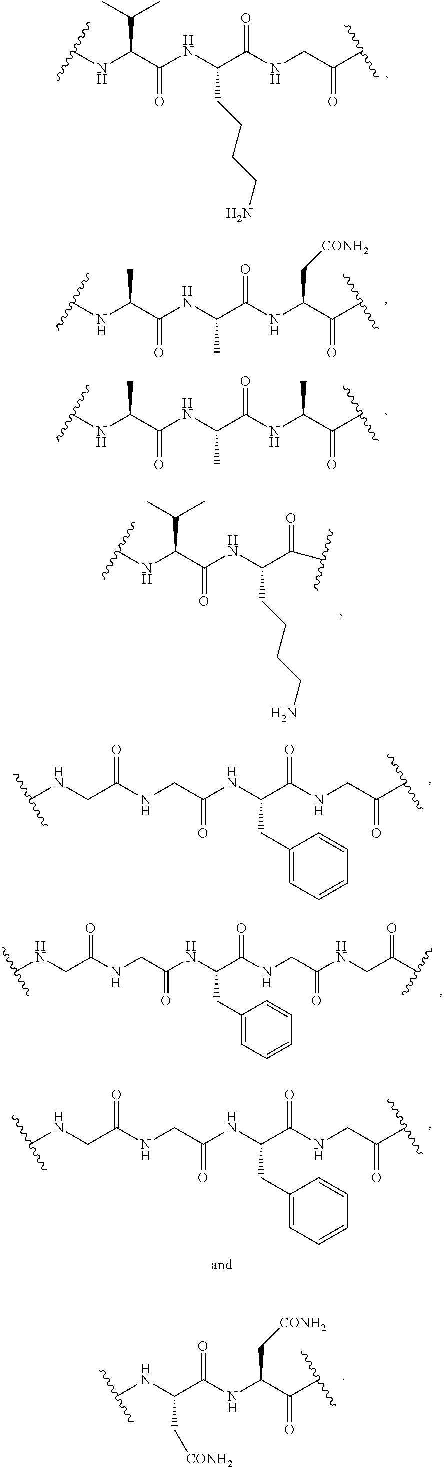

In certain embodiments of Formula (III)—(IVB), including any of the foregoing, L³ is —C(O)—.

In certain embodiments of Formula (III)—(IVB), including any of the foregoing, L³ is —C(O)—Z—(CR^{a}R^{b})ₐ—C(O)—Z-L⁴-OC(O)— wherein L⁴ is

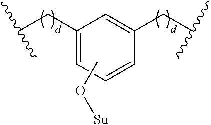

and Su is a hexose form of a monosaccharide and d is an integer independently selected from 1, 2, and 3. In certain embodiments of Formula (III)—(IVB), including any of the foregoing, L³ is —C(O)—NR²—(CR^{a}R^{b})ₐ—C(O)—NR²-L⁴-OC(O)—. In certain embodiments of Formula (III)—(IVB), including any of the foregoing, L³ is —C(O)—NR²—(CH₂)₂—C(O)—NR²-L⁴-OC(O)—. In certain embodiments of Formula (III)—(IVB), including any of the foregoing, L³ is —C(O)—NH—(CR^{a}R^{b})ₐ—C(O)—NH-L⁴-OC(O)—. In certain embodiments of Formula (III)—(IVB), including any of the foregoing, L³ is —C(O)—NH—(CH₂)₂—C(O)—NH-L⁴-OC(O)—.

In certain embodiments of Formula (III)—(IVB), including any of the foregoing, L⁴ is

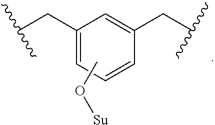

In certain embodiments of Formula (III)—(IVB), including any of the foregoing, L⁴ is

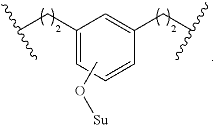

In certain embodiments of Formula (III)—(IVB), including any of the foregoing, L⁴ is

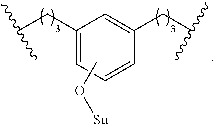

In some embodiments of Formula (III)—(IVB), including any of the foregoing, Su is a sugar moiety. In some embodiments, Su is a hexose form of a monosaccharide. Su may be a glucuronic acid or mannose residue. In certain embodiments of Formula (III)—(IVB), including any of the foregoing, Su is

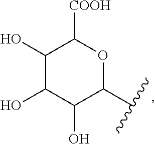

wherein [symbol] represents attachment to the remainder of the compound. In certain embodiments of Formula (III)—(IVB), including any of the foregoing, Su is

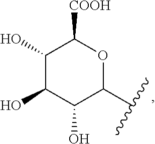

wherein [symbol] represents attachment to the remainder of the compound.

In certain embodiments of Formula (III)—(IVB), including any of the foregoing, L⁴ is

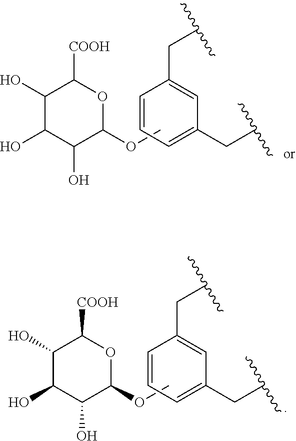

In certain embodiments of Formula (III)—(IVB), including any of the foregoing, L³ is —C(O)—NH—(CH₂)₂—C(O)—NH-L⁴-OC(O)— wherein L⁴ is

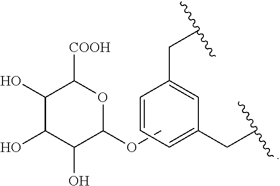

In certain embodiments of Formula (III)—(IVB), including any of the foregoing, L³ is —C(O)—NH—(CH₂)₂—C(O)—NH-L⁴-OC(O)— wherein L⁴ is

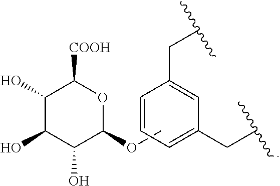

In certain embodiments of Formula (III)—(IVB), including any of the foregoing, L² is —(CR^{a}R^{b})ₐ-POLY¹- and L³ is —C(O)-AA-. In certain embodiments of Formula (III)—(IVB), including any of the foregoing, L² is —(CR^{a}R^{b})ₐ-POLY¹-; L³ is is —C(O)-AA-; and, POLY¹ is

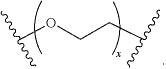

In certain embodiments of Formula (III)—(IVB), including any of the foregoing, L² is —(CH₂)ₐ-POLY¹-; L³ is —C(O)-AA-; POLY¹ is

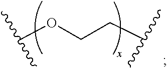

and x is an integer between 10 and 15. In certain embodiments of Formula (III)—(IVB), including any of the foregoing, L² is —(CH₂)₂-POLY¹-; L³ is —C(O)-AA-; and, POLY¹ is

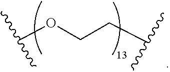

In certain embodiments of Formula (III)—(IVB), including any of the foregoing, L² is —(CH₂)₂-POLY¹-; L³ is —C(O)-AA-; POLY¹ is

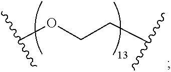

and AA is a a dipeptide residue, a tripeptide residue, a tetrapeptide residue, or a pentapeptide residue.

In certain embodiments of Formula (III)—(IVB), including any of the foregoing, L² is —(CR^{a}R^{b})ₐ-POLY¹- and L³ is —C(O)-AA-Z—(CR^{a}R^{b})ₐ—Z—(CR^{a}R^{b})ₐ—C(O)—. In certain embodiments of Formula (III)—(IVB), including any of the foregoing, L² is —(CR^{a}R^{b})ₐ-POLY¹-; L³ is —C(O)-AA-Z—(CR^{a}R^{b})ₐ—Z—(CR^{a}R^{b})ₐ—C(O)—; POLY¹ is

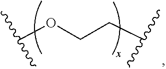

and Z is —NH—. In certain embodiments of Formula (III)—(IVB), including any of the foregoing, L² is —(CH₂)ₐ-POLY¹-; L³ is —C(O)-AA-NH—(CH₂)ₐ—NH—(CH₂)ₐ—C(O)—; POLY¹ is

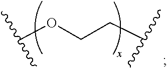

and x is an integer between 10 and 15. In certain embodiments of Formula (III)—(IVB), including any of the foregoing, L² is —(CH₂)₂-POLY¹-; L₃ is —C(O)-AA-NH—CH₂—NH—CH₂—C(O)—; and, POLY¹ is

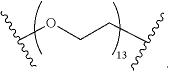

In certain embodiments of Formula (III)—(IVB), including any of the foregoing, L² is —(CH₂)₂-POLY¹-; L³ is —C(O)-AA-NH—CH₂—NH—CH₂—C(O)—; POLY¹ is

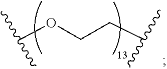

and AA is a a dipeptide residue, a tripeptide residue, a tetrapeptide residue, or a pentapeptide residue.

In certain embodiments of Formula (III)—(IVB), including any of the foregoing, L² is —(CR^{a}R^{b})ₐ-POLY¹- and L³ is —C(O). In certain embodiments of Formula (III)—(IVB), including any of the foregoing, L² is —(CR^{a}R^{b})ₐ-POLY¹-; is —C(O); and, POLY¹ is

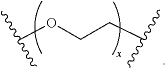

In certain embodiments of Formula (III)—(IVB), including any of the foregoing, L² is —(CH₂)ₐ-POLY¹-; L³ is —C(O); POLY¹ is

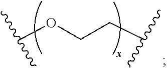

and x is an integer between 10 and 15. In certain embodiments of Formula (III)—(IVB), including any of the foregoing, L² is —(CH₂)₂-POLY¹-; L³ is —C(O); and, POLY¹ is

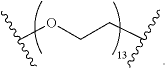

In certain embodiments of Formula (III)—(IVB), including any of the foregoing, L² is —(CH₂)₂-POLY¹-; L₃ is —C(O); POLY¹ is

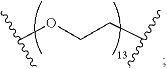

and AA is a dipeptide residue, a tripeptide residue, a tetrapeptide residue, or a pentapeptide residue.

In certain embodiments of Formula (III)—(IVB), including any of the foregoing, L² is —(CR^{a}R^{b})ₐ-POLY¹- and L³ is absent. In certain embodiments of Formula (III)—(IVB), including any of the foregoing, L² is —(CR^{a}R^{b})ₐ-POLY¹-; L³ is absent; and, POLY¹ is

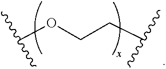

In certain embodiments of Formula (III)—(IVB), including any of the foregoing, L² is —(CH₂)ₐ-POLY¹-; L³ is absent; POLY¹ is

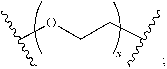

and x is an integer between 10 and 15. In certain embodiments of Formula (III)—(IVB), including any of the foregoing, L² is —(CH₂)₂-POLY¹-; L³ is absent; and, POLY¹ is

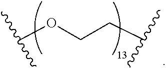

In certain embodiments of Formula (III)—(IVB), including any of the foregoing, L² is —(CH₂)₂-POLY¹-; L³ is absent; POLY¹ is

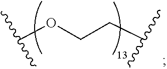

and AA is a dipeptide residue, a tripeptide residue, a tetrapeptide residue, or a pentapeptide residue.

In certain embodiments of Formula (III)—(IVB), including any of the foregoing, L² is —(CR^{a}R^{b})ₐ-POLY¹- and L³ is —C(O)-AA-Z—(CR^{a}R^{b})ₐ—. In certain embodiments of Formula (III)—(IVB), including any of the foregoing, L² is —(CR^{a}R^{b})ₐ-POLY¹-; L³ is is —C(O)-AA-Z—(CR^{a}R^{b})ₐ—; and, POLY¹ is

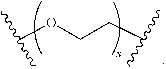

In certain embodiments of Formula (III)—(IVB), including any of the foregoing, L² is —(CH₂)ₐ-POLY¹-; —C(O)-AA-NR²—(CR^{a}R^{b})ₐ—; POLY¹ is

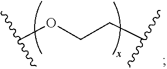

and x is an integer between 10 and 15. In certain embodiments of Formula (III)—(IVB), including any of the foregoing, L² is —(CH₂)₂-POLY¹-; L³ is —C(O)-AA-NH—(CR^{a}R^{b})ₐ—; and, POLY¹ is

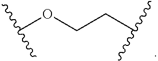 [SMILES: *CCO*]

In certain embodiments of Formula (III)—(IVB), including any of the foregoing, L² is —(CH₂)₂-POLY¹-; L³ is —C(O)-AA-NH—(CR^{a}R^{b})ₐ—; POLY¹ is

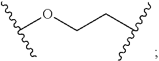 [SMILES: *CCO*]

and AA is a dipeptide residue, a tripeptide residue, a tetrapeptide residue, or a pentapeptide residue.

Non-limiting examples of -L²-L³- include:

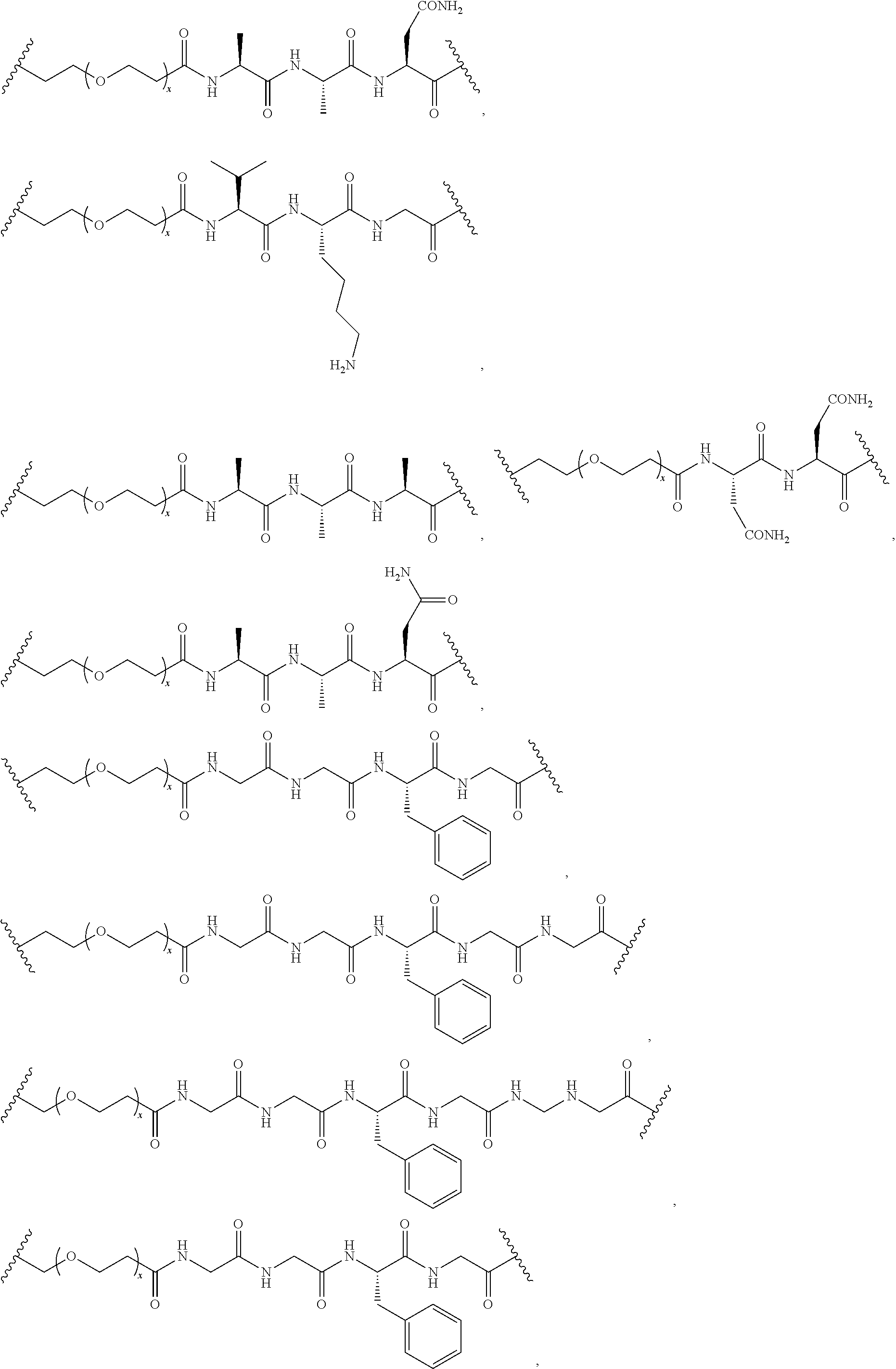

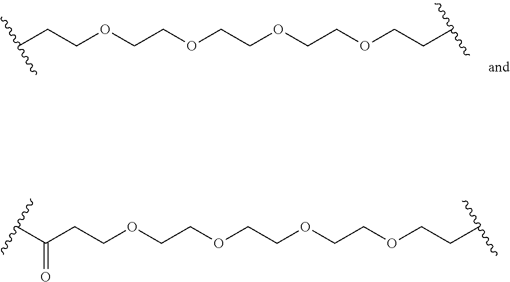 [SMILES: *CCOCCOCCOCCOCC* ; *CCOCCOCCOCCOCCC(*)=O]

Additional non-limiting examples of -L²-L³- include:

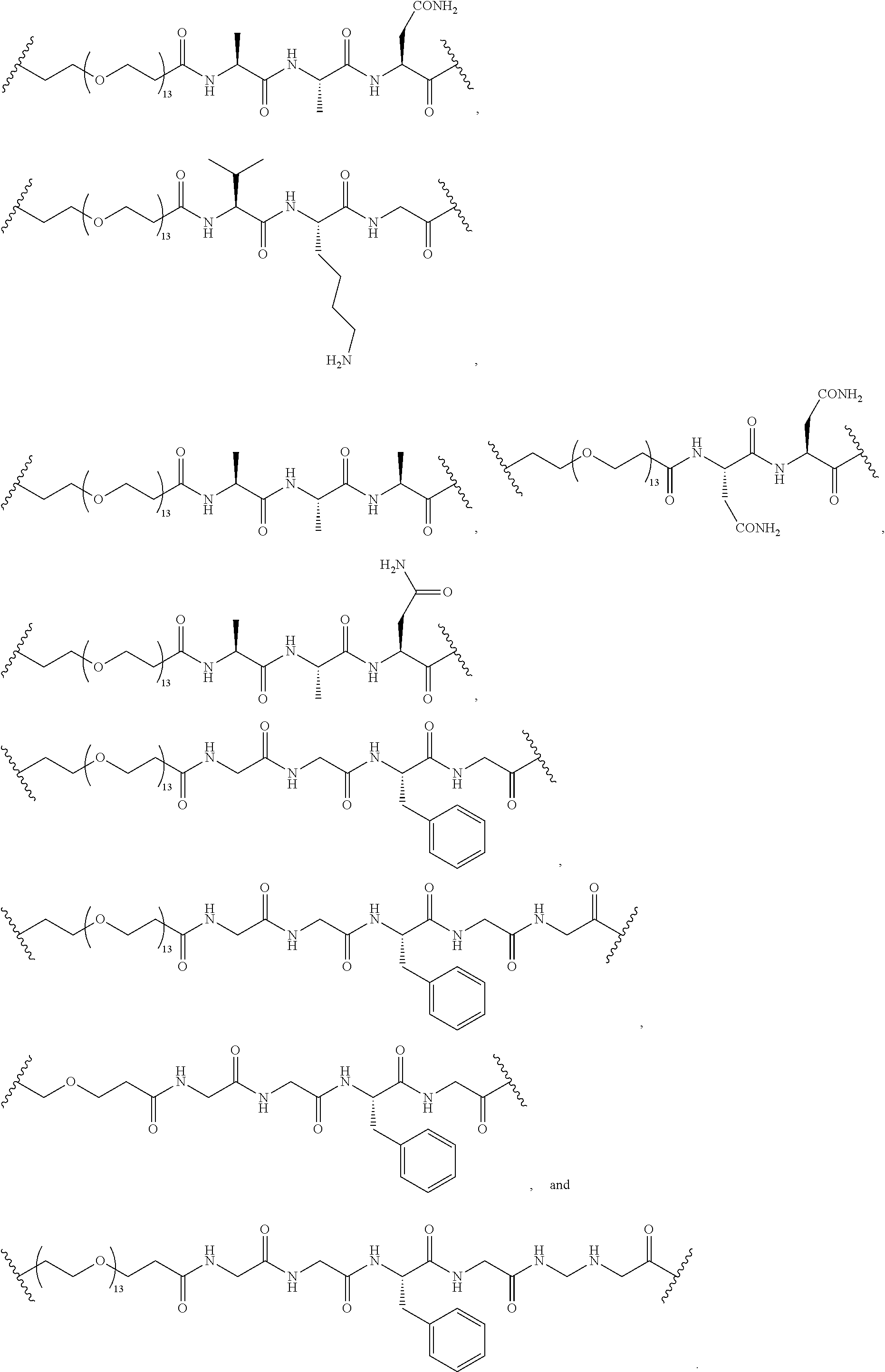

In certain embodiments of Formula (III)—(IVB), including any of the foregoing, L² is

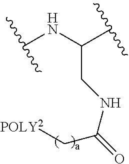

and L³ is —C(O)—Z—(CR^{a}R^{b})ₐ—C(O)—Z-L⁴-OC(O)— wherein L⁴ is

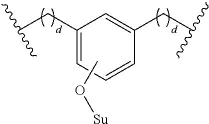

and Su is a hexose form of a monosaccharide and d is an integer independently selected from 1, 2, and 3. In certain embodiments of Formula (III)—(IVB), including any of the foregoing, L² is

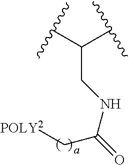

and L³ is —C(O)—Z—(CR^{a}R^{b})ₐ—C(O)—Z-L⁴-OC(O)—.

In certain embodiments of Formula (III)—(IVB), including any of the foregoing, L² is

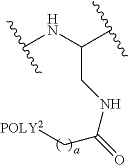

and L³ is —C(O). In certain embodiments, including any of the foregoing, L² is

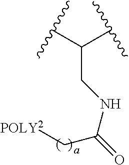

and L³ is absent.

In certain embodiments of Formula (III)—(IVB), including any of the foregoing, L² is selected from the group consisting of

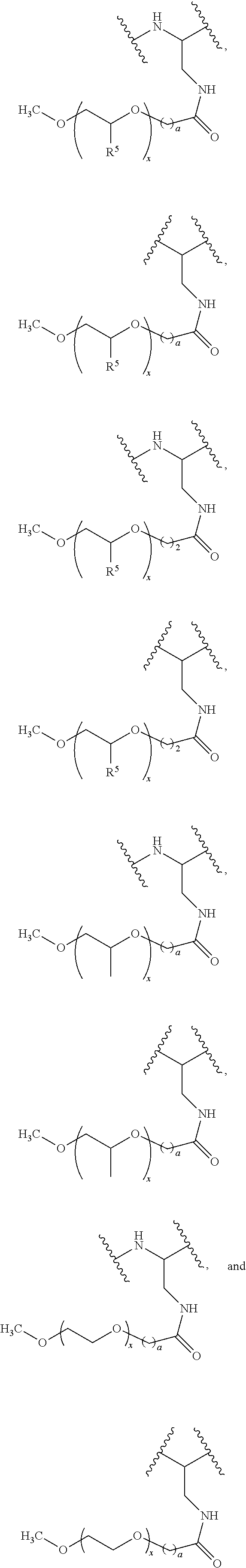

and L³ is —C(O)—NH—(CR^{a}R^{b})ₐ—C(O)—NH-L⁴-OC(O)—.

In certain embodiments of Formula (III)—(IVB), including any of the foregoing, L² is selected from the group consisting of

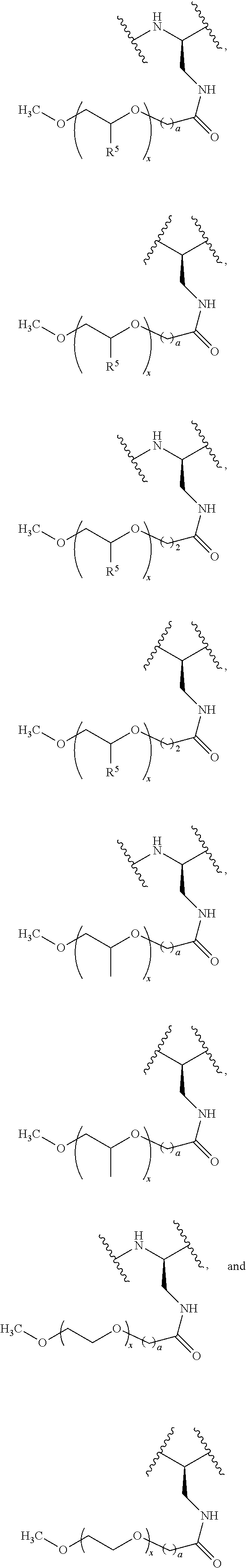

and L³ is —C(O)—NH—(CR^{a}R^{b})ₐ—C(O)—NH-L⁴-OC(O)—.

In certain embodiments of Formula (III)—(IVB), including any of the foregoing, L² is selected from the group consisting of

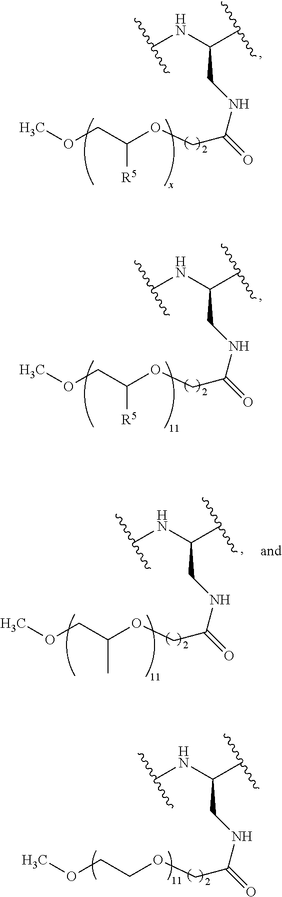

and L³ is —C(O)—NH—(CH₂)₂—C(O)—NH-L⁴-OC(O)— wherein L⁴ is

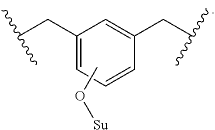

In certain embodiments of Formula (III)—(IVB), including any of the foregoing, L² is selected from the group consisting of

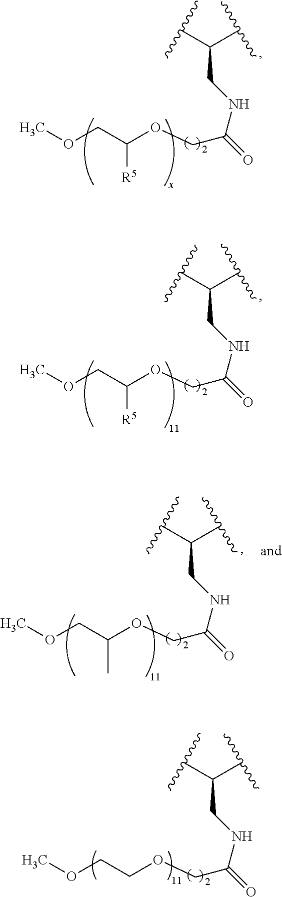

### and L³ is —C(O).

In certain embodiments, including any of the foregoing, D is a cytotoxic payload selected from a tubulin inhibitor, a DNA topoisomerase I inhibitor, and a DNA topoisomerase II inhibitor. In some embodiments, including any of the foregoing, D is a tubulin inhibitor. In some embodiments, including any of the foregoing, D is a DNA topoisomerase I inhibitor. In some embodiments, including any of the foregoing, D is a DNA topoisomerase I inhibitor selected from the group consisting of irinotecan, SN-38, topotecan, exatecan. In some embodiments, including any of the foregoing, D is irinotecan. In some embodiments, including any of the foregoing, D is SN-38. In some embodiments, including any of the foregoing, D is topotecan. In some embodiments, including any of the foregoing, D is exatecan. In some embodiments, including any of the foregoing, D is a DNA topoisomerase II inhibitor. In some embodiments, including any of the foregoing, D is a DNA topoisomerase II inhibitor selected from the group consisting of etoposide, teniposide, and tafluposide. In some embodiments, including any of the foregoing, D is etoposide. In some embodiments, including any of the foregoing, D is teniposide. In some embodiments, including any of the foregoing, D is tafluposide. In some embodiments, including any of the foregoing, D is selected from the group consisting of hemiasterlins, camptothecins, and anthracyclines. Anthracyclines may include PNU-159682 and EDA PNU-159682 derivatives. In some embodiments, including any of the foregoing, D is an anthracycline selected from the group consisting of daunorubicin, doxorubicin, epirubicin, idarubicin, mitoxantrone, and valrubicin. In some embodiments, including any of the foregoing, D is daunorubicin. In some embodiments, including any of the foregoing, D is doxorubicin. In some embodiments, including any of the foregoing, D is epirubicin. In some embodiments, including any of the foregoing, D is idarubicin. In some embodiments, including any of the foregoing, D is mitoxantrone. In some embodiments, including any of the foregoing, D is valrubicin. In some embodiments, including any of the foregoing, D is a hemiasterlin. In some embodiments, including any of the foregoing, D is a camptothecin. In some embodiments, including any of the foregoing, D is an anthracycline. In some embodiments, including any of the foregoing, D is PNU-159682. In some embodiments, including any of the foregoing, D is an EDA PNU compound. In some embodiments, including any of the foregoing, D is an EDA PNU-159682 derivative. In some embodiments, including any of the foregoing, D is hemiasterlin, exatecan, PNU-159682, or an EDA PNU-159682 derivative. In some embodiments, including any of the foregoing, D is hemiasterlin. In some embodiments, including any of the foregoing, D is exatecan. In some embodiments, including any of the foregoing, D is of PNU-159682. In some embodiments, including any of the foregoing, D is EDA PNU-159682 compound or derivative.

In some embodiments, including any of the foregoing, D is an alkylating agent. In some embodiments, including any of the foregoing, D is a bifunctional alkylator. In some embodiments, including any of the foregoing, D is a bifunctional alkylator selected from the group consisting of cyclophosphamide, mechlorethamine, chlorambucil, and melphalan. In some embodiments, including any of the foregoing, D is cyclophosphamide. In some embodiments, including any of the foregoing, D is mechlorethamine. In some embodiments, including any of the foregoing, D is chlorambucil. In some embodiments, including any of the foregoing, D is melphalan. In some embodiments, including any of the foregoing, D is a monofunctional alkylator. In some embodiments, including any of the foregoing, D is a monofunctional alkylator selected from the group consisting of dacarbazine, nitrosourea, and temozolomide. In some embodiments, including any of the foregoing, D is dacarbazine. In some embodiments, including any of the foregoing, D is nitrosourea. In some embodiments, including any of the foregoing, D is temozolomide. In some embodiments, including any of the foregoing, D is a cytoskeletal disruptor (e.g., a taxane). In some embodiments, including any of the foregoing, D is a cytoskeletal disruptor selected from the group consisting of paclitaxel, docetaxel, abraxane, and taxotere. In some embodiments, including any of the foregoing, D is paclitaxel. In some embodiments, including any of the foregoing, D is docetaxel. In some embodiments, including any of the foregoing, D is abraxane. In some embodiments, including any of the foregoing, D is taxotere. In some embodiments, including any of the foregoing, D is an epothilone. In some embodiments, including any of the foregoing, D is an epothilone selected from the group consisting of epothilone A, epothilone B, epothilone C, epothilone D, and ixabepilone. In some embodiments, including any of the foregoing, D is epothilone A. In some embodiments, including any of the foregoing, D is epothilone B. In some embodiments, including any of the foregoing, D is epothilone C. In some embodiments, including any of the foregoing, D is epothilone D. In some embodiments, including any of the foregoing, D is ixabepilone. In some embodiments, including any of the foregoing, D is a histone deacetylase inhibitor. In some embodiments, including any of the foregoing, D is a histone deacetylase inhibitor selected from the group consisting of vorinostat and romidepsin. In some embodiments, including any of the foregoing, D is vorinostat. In some embodiments, including any of the foregoing, D is romidepsin. In some embodiments, including any of the foregoing, D is a kinase inhibitor. In some embodiments, including any of the foregoing, D is a kinase inhibitor selected from the group consisting of bortezomib, erlotinib, gefitinib, imatinib, vemurafenib, and vismodegib. In some embodiments, including any of the foregoing, D is bortezomib. In some embodiments, including any of the foregoing, D is erlotinib. In some embodiments, including any of the foregoing, D is gefitinib. In some embodiments, including any of the foregoing, D is imatinib. In some embodiments, including any of the foregoing, D is vemurafenib. In some embodiments, including any of the foregoing, D is vismodegib. In some embodiments, including any of the foregoing, D is a nucleotide analog and/or precursor analog. In some embodiments, including any of the foregoing, D is a nucleotide analog and/or precursor analog selected from the group consisting of azacitidine, azathioprine, capecitabine, cytarabine, doxifluridine, fluorouracil, gemcitabine, hydroxyurea, mercaptopurine, methotrexate, and tioguanine (formerly thioguanine). In some embodiments, including any of the foregoing, D is azacitidine. In some embodiments, including any of the foregoing, D is azathioprine. In some embodiments, including any of the foregoing, D is capecitabine. In some embodiments, including any of the foregoing, D is cytarabine. In some embodiments, including any of the foregoing, D is doxifluridine. In some embodiments, including any of the foregoing, D is fluorouracil. In some embodiments, including any of the foregoing, D is gemcitabine. In some embodiments, including any of the foregoing, D is hydroxyurea. In some embodiments, including any of the foregoing, D is mercaptopurine. In some embodiments, including any of the foregoing, D is methotrexate. In some embodiments, including any of the foregoing, D is tioguanine (formerly thioguanine). In some embodiments, including any of the foregoing, D is a peptide antibiotic. In some embodiments, including any of the foregoing, D is a peptide antibiotic selected from the group consisting of bleomycin and actinomycin. In some embodiments, including any of the foregoing, D is bleomycin. In some embodiments, including any of the foregoing, D is actinomycin. In some embodiments, including any of the foregoing, D is a platinum-based agent. In some embodiments, including any of the foregoing, D is a platinum-based agent selected from the group consisting of carboplatin, cisplatin, and oxaliplatin. In some embodiments, including any of the foregoing, D is carboplatin. In some embodiments, including any of the foregoing, D is cisplatin. In some embodiments, including any of the foregoing, D is oxaliplatin. In some embodiments, including any of the foregoing, D is a retinoid. In some embodiments, including any of the foregoing, D is a retinoid selected from the group consisting of tretinoin, alitretinoin, and bexarotene. In some embodiments, including any of the foregoing, D is tretinoin. In some embodiments, including any of the foregoing, D is alitretinoin. In some embodiments, including any of the foregoing, D is bexarotene. In some embodiments, including any of the foregoing, D is a vinca alkaloid and derivatives thereof. In some embodiments, including any of the foregoing, D is a vinca alkaloid and derivatives thereof selected from the group consisting of vinblastine, vincristine, vindesine, vinorelbine. In some embodiments, including any of the foregoing, D is a residue of vinblastine. In some embodiments, including any of the foregoing, D is vincristine. In some embodiments, including any of the foregoing, D is vindesine.

In any of the foregoing embodiments, the conjugate comprises n2 number of linker-payloads, wherein n2 is an integer from 1 to 10. In some embodiments, n2 is 2. In some embodiments, n2 is 3. In some embodiments, n2 is 4. In some embodiments, n2 is 5. In some embodiments, n2 is 6. In some embodiments, n2 is 7. In some embodiments, n2 is 8. In some embodiments, n2 is 9. In some embodiments, n2 is 10.

In some embodiments, provided herein are anti-Tissue Factor conjugates having the structure of any of Conjugates A-E in the table below. In some embodiments, n2 is an integer from 1 to 8. In some embodiments, n2 is 2. In some embodiments, n2 is 4. In some embodiments, n2 is 6. In some embodiments, n2 is 8. The present disclosure encompasses each and every regioisomer of the conjugate structures depicted below.

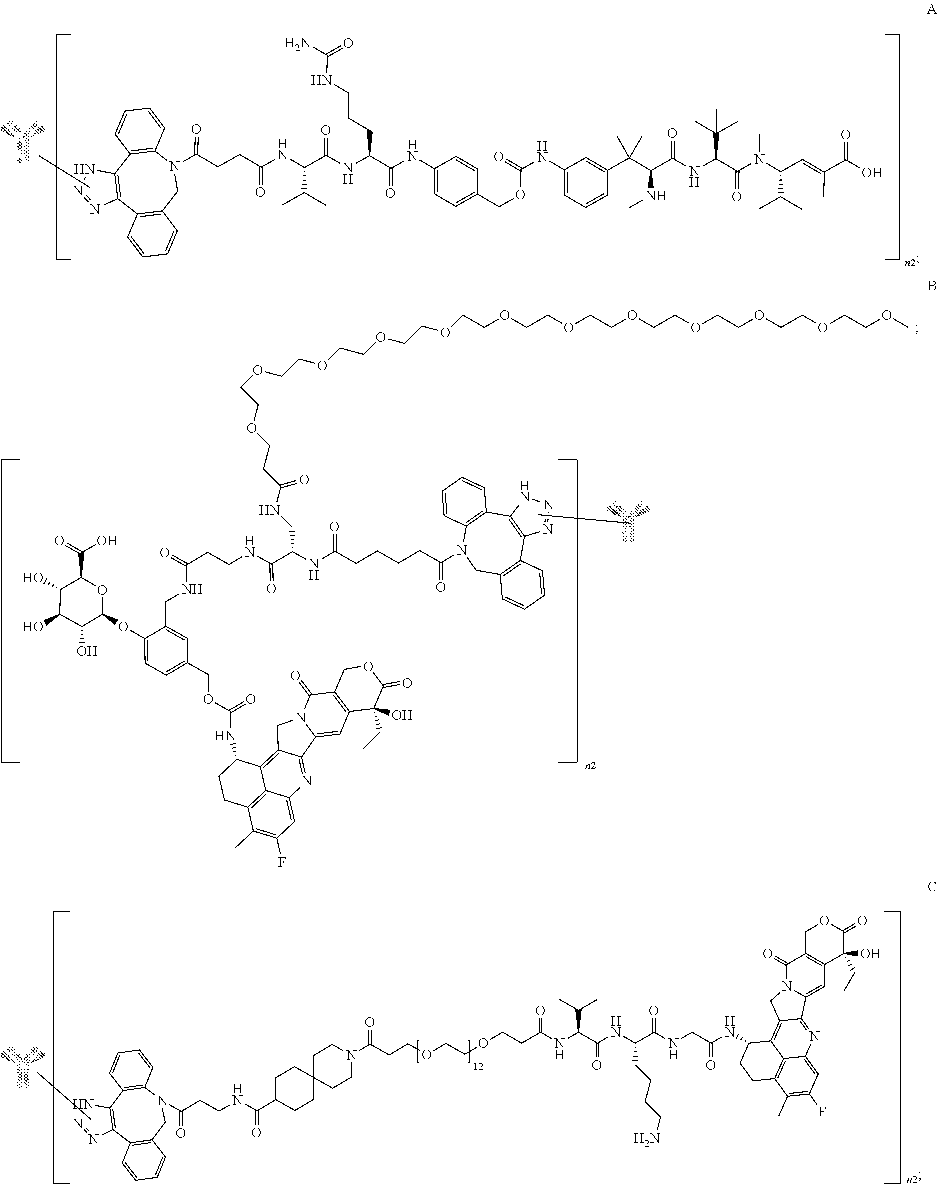

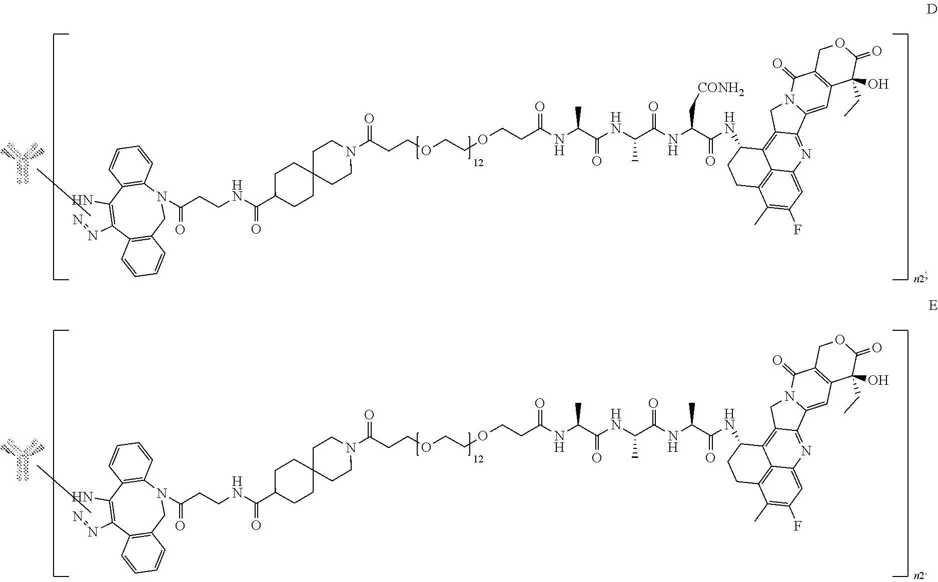

In some embodiments, provided herein are anti-Tissue Factor conjugates having the structure of any of Conjugates A1-E1 in the table below. In some embodiments, n2 is an integer from 1 to 8. In some embodiments, n2 is 2. In some embodiments, n2 is 4. In some embodiments, n2 is 6. In some embodiments, n2 is 8. The present disclosure encompasses each and every regioisomer of the conjugate structures depicted below.

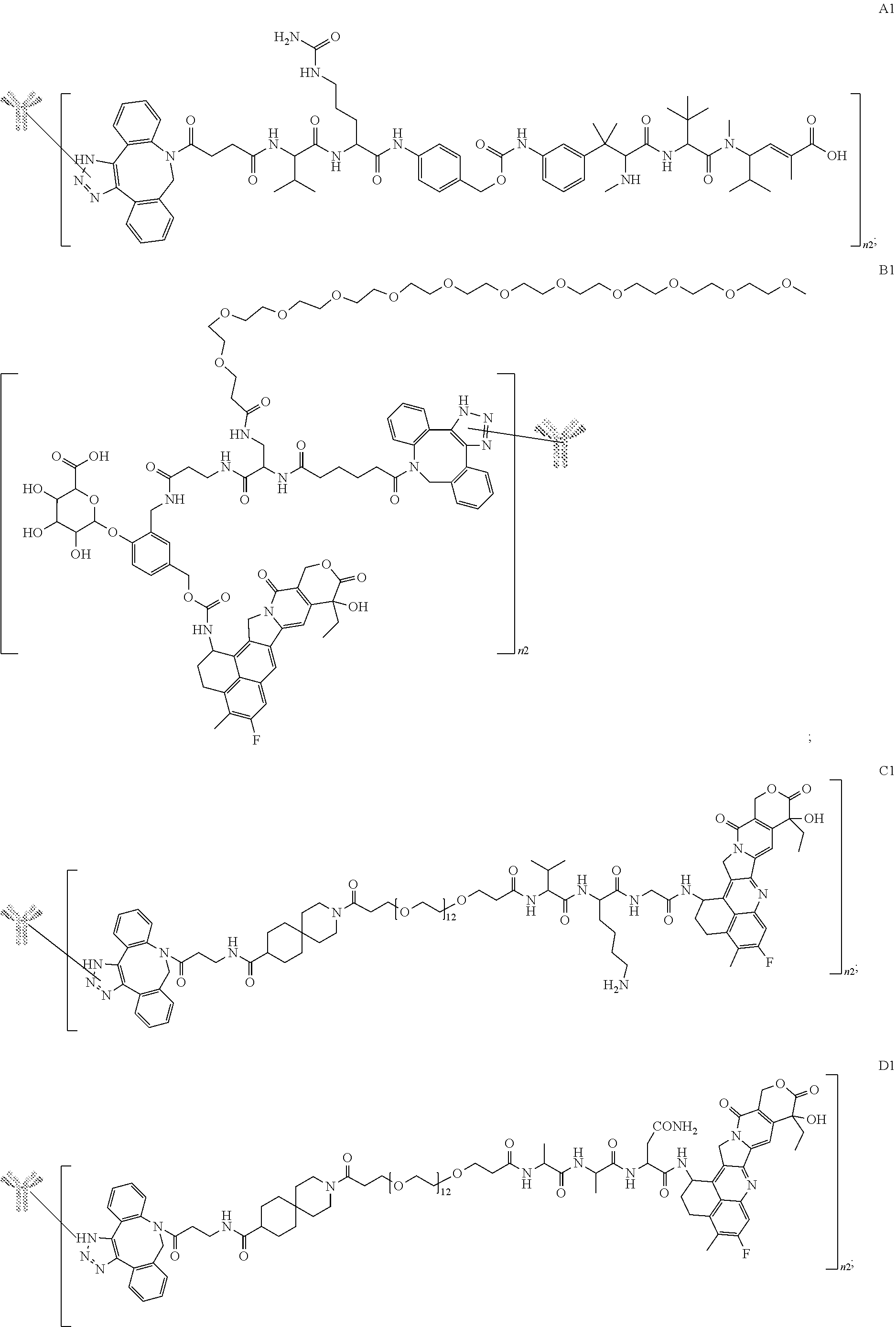

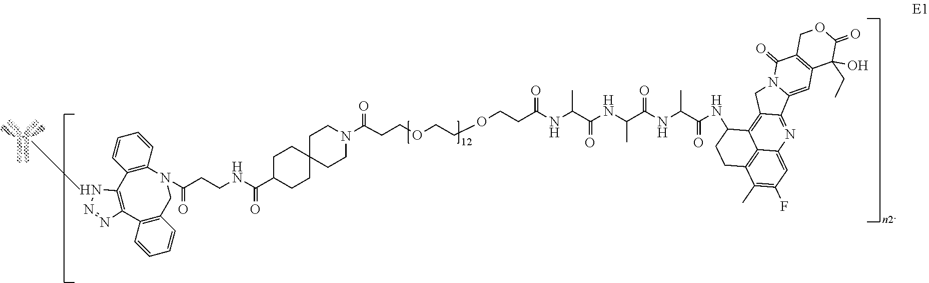

It should be understood that in certain embodiments, other linker payloads and conjugation structures are provided such as those discussed in International Patent Application No. PCT/US2023/023688, filed Jun. 27, 2023 entitled β-Glucuronide Linker-Payloads, Protein Conjugates Thereof, And Methods Thereof as well as U.S. Provisional Application No. 61/516,579, filed Jul. 31, 2023, entitled STING Agonist Compounds and Conjugates, each of which is incorporated herein by reference in its entirety. In some embodiments provided herein are anti-Tissue Factor conjugates comprising an antibody described herein linked to one or more linker-payloads as described in PCT Application PCT/US2023/26338, PCT/US/2020/031052, PCT/US/2018/051364, PCT/US2018/051322, PCT/US2017/015501, PCT/US2017/015503, and PCT/US2016/015844, each of which are incorporated by reference in its entirety.

In any of the foregoing embodiments wherein the anti-Tissue Factor conjugate has a structure according to any one of Conjugates A-E, the bracketed structure can be covalently bonded to one or more non-natural amino acids of the antibody, wherein the one or more non-natural amino acids are located at sites selected from the group consisting of: HC—F404, HC—Y180, HC—F241, LC-K42, and LC-E161, according to the Kabat or EU numbering scheme of Kabat. In some embodiments, the bracketed structure is covalently bonded to one or more non-natural amino acids at site HC—F404 of the antibody. In some embodiments, the bracketed structure is covalently bonded to one or more non-natural amino acids at site HC—Y180 of the antibody. In some embodiments, the bracketed structure is covalently bonded to one or more non-natural amino acids at site HC—F241 of the antibody. In some embodiments, the bracketed structure is covalently bonded to one or more non-natural amino acids at site LC-K42 of the antibody. In some embodiments, the bracketed structure is covalently bonded to one or more non-natural amino acids at site LC-E161 of the antibody. In some embodiments, the bracketed structures are covalently bonded to non-natural amino acids at sites HC—F404 and HC—Y180 of the antibody. In some embodiments, the bracketed structures are covalently bonded to non-natural amino acids at sites HC—F404, HC—Y180, and LC-K42 of the antibody. In some embodiments, the bracketed structures are covalently bonded to non-natural amino acids at sites HC—F404, HC—Y180, LC-K42, and LC-E161 of the antibody. In some embodiments, the bracketed structures are covalently bonded to non-natural amino acids at sites HC—F404, HC—Y180, and HC—F241 of the antibody. In some embodiments, the bracketed structures are covalently bonded to non-natural amino acids at sites HC—F404, HC—Y180, HC—F241, and LC-K42 of the antibody. In some embodiments, the bracketed structures are covalently bonded to non-natural amino acids at sites HC—F404 and HC—F241.

In particular embodiments, provided herein are anti-Tissue Factor conjugates according to any of the conjugates described herein wherein COMP indicates a residue of the non-natural amino acid according to Formula (30), below. In particular embodiments, provided herein are anti-Tissue Factor conjugates according to any of the conjugates described herein wherein COMP indicates a residue of the non-natural amino acid according to Formula (30), below, at heavy chain position 404 according to the EU numbering system. In particular embodiments, provided herein are anti-Tissue Factor conjugates according to any of the conjugates described herein wherein COMP indicates a residue of the non-natural amino acid according to Formula (30), below, at heavy chain position 180 according to the EU numbering system. In particular embodiments, provided herein are anti-Tissue Factor conjugates according to any of the conjugates described herein wherein COMP indicates a residue of the non-natural amino acid according to Formula (30), below, at heavy chain position 241 according to the EU numbering system. In particular embodiments, provided herein are anti-Tissue Factor conjugates according to any of the conjugates described herein wherein COMP indicates a residue of the non-natural amino acid according to Formula (30), below, at heavy chain position 222 according to the EU numbering system. In particular embodiments, provided herein are anti-Tissue Factor conjugates according to any of the conjugates described herein wherein COMP indicates a residue of the non-natural amino acid according to Formula (30), below, at light chain position 7 according to the Kabat or Chothia numbering system. In particular embodiments, provided herein are anti-Tissue Factor conjugates according to any of the conjugates described herein wherein COMP indicates a residue of the non-natural amino acid according to Formula (30), below, at light chain position 42 according to the Kabat or Chothia numbering system. In certain embodiments, PAY is selected from the group consisting of maytansine, hemiasterlin, amanitin, camptothecin, exatecan, exatecan derivative (DXd), SN-38, anthracycline, PNU-159682, PNU derivative (PNU-EDA), pyrrolobenzodiazepine (PBD), MMAF, and MMAE. In certain embodiments, PAY is maytansine. In certain embodiments, PAY is hemiasterlin. In certain embodiments, PAY is amanitin. In certain embodiments, PAY is exatecan. In certain embodiments, PAY is exatecan derivative Dxd. In certain embodiments, PAY is anthracycline. In certain embodiments, PAY is PNU-159682. In certain embodiments, PAY is PNU derivative (PNU-EDA). In certain embodiments, PAY is pyrrolobenzodiazepine. In certain embodiments, PAY is MMAF. In certain embodiments, PAY is MMAE.

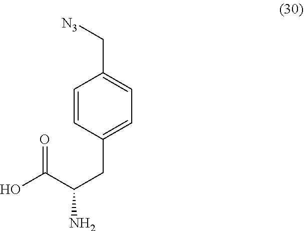 [SMILES: [N-]=[N+]=NCc1ccc(C[C@H](N)C(=O)O)cc1]

In particular embodiments, provided herein are anti-Tissue Factor conjugates according to any of the conjugates described herein wherein COMP indicates a residue of the non-natural amino acid according to Formula (56), below. In particular embodiments, provided herein are anti-Tissue Factor conjugates according to any of the conjugates described herein wherein COMP indicates a residue of the non-natural amino acid according to Formula (56), below, at heavy chain position 404 according to the EU numbering system. In particular embodiments, provided herein are anti-Tissue Factor conjugates according to any of the conjugates described herein wherein COMP indicates a residue of the non-natural amino acid according to Formula (56), below, at heavy chain position 180 according to the EU numbering system. In particular embodiments, provided herein are anti-Tissue Factor conjugates according to any of the conjugates described herein wherein COMP indicates a residue of the non-natural amino acid according to Formula (56), below, at heavy chain position 241 according to the EU numbering system. In particular embodiments, provided herein are anti-Tissue Factor conjugates according to any of the conjugates described herein wherein COMP indicates a residue of the non-natural amino acid according to Formula (56), below, at heavy chain position 222 according to the EU numbering system. In particular embodiments, provided herein are anti-Tissue Factor conjugates according to any of the conjugates described herein wherein COMP indicates a residue of the non-natural amino acid according to Formula (56), below, at light chain position 7 according to the Kabat or Chothia numbering system. In particular embodiments, provided herein are anti-Tissue Factor conjugates according to any of the conjugates described herein wherein COMP indicates a residue of the non-natural amino acid according to Formula (56), below, at light chain position 42 according to the Kabat or Chothia numbering system. In certain embodiments, PAY is selected from the group consisting of maytansine, hemiasterlin, amanitin, camptothecin, exatecan, exatecan derivative (DXd), SN-38, anthracycline, PNU-159682, PNU derivative (PNU-EDA), pyrrolobenzodiazepine (PBD), MMAF, and MMAE. In certain embodiments, PAY is maytansine. In certain embodiments, PAY is hemiasterlin. In certain embodiments, PAY is amanitin. In certain embodiments, PAY is exatecan. In certain embodiments, PAY is exatecan derivative (Dxd). In certain embodiments, PAY is deruxtecan. In certain embodiments, PAY is anthracycline. In certain embodiments, PAY is PNU-159682. In certain embodiments, PAY is PNU derivative (PNU-EDA). In certain embodiments, PAY is pyrrolobenzodiazepine. In certain embodiments, PAY is MMAF. In certain embodiments, PAY is MMAE.

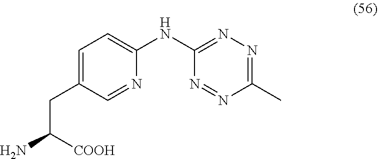 [SMILES: Cc1nnc(Nc2ccc(C[C@H](N)C(=O)O)cn2)nn1]

In particular embodiments, provided herein are anti-Tissue Factor conjugates according to any of the conjugates described herein wherein COMP indicates a non-natural amino acid residue of para-azido-L-phenylalanine. In particular embodiments, provided herein are anti-Tissue Factor conjugates according to any of the conjugates described herein wherein COMP indicates the non-natural amino acid residue para-azido-phenylalanine at heavy chain position 404 according to the EU numbering system. In particular embodiments, provided herein are anti-Tissue Factor conjugates according to any of the conjugates described herein wherein COMP indicates a non-natural amino acid residue of para-azido-L-phenylalanine at heavy chain position 180 according to the EU numbering system. In particular embodiments, provided herein anti-Tissue Factor conjugates according to any of the conjugates described herein wherein COMP indicates a non-natural amino acid residue para-azido-L-phenylalanine at heavy chain position 241 according to the EU numbering system. In particular embodiments, provided herein are anti-Tissue Factor conjugates according to any of the conjugates described herein wherein COMP indicates a non-natural amino acid residue para-azido-L-phenylalanine at heavy chain position 222 according to the EU numbering system. In particular embodiments, provided herein are anti-Tissue Factor conjugates according to any of the conjugates described herein wherein COMP indicates a non-natural amino acid residue para-azido-L-phenylalanine at light chain position 7 according to the Kabat or Chothia numbering system. In particular embodiments, provided herein are anti-Tissue Factor conjugates according to any of the conjugates described herein wherein COMP indicates a non-natural amino acid residue para-azido-L-phenylalanine at light chain position 42 according to the Kabat or Chothia numbering system. In certain embodiments, PAY is selected from the group consisting of maytansine, hemiasterlin, amanitin, camptothecin, exatecan, exatecan derivative (DXd), SN-38, anthracycline, PNU-159682, pyrrolobenzodiazepine (PBD), MMAF, and MMAE. In certain embodiments, PAY is maytansine. In certain embodiments, PAY is hemiasterlin. In certain embodiments, PAY is amanitin. In certain embodiments, PAY is exatecan. In certain embodiments, PAY is exatecan derivative (Dxd). In certain embodiments, PAY is anthracycline. In certain embodiments, PAY is PNU-159682. In certain embodiments, PAY is PNU derivative (PNU-EDA). In certain embodiments, PAY is pyrrolobenzodiazepine. In certain embodiments, PAY is MMAF. In certain embodiments, PAY is MMAE.

It is expected without being bound to theory that the antibody conjugates of the present disclosure can avoid Factor X inhibition and bleeding issues by selection of an antibody that does not interfere with Tissue Factor binding to coagulation factors. The exatecan payload is believed to inhibit TOPO-1 causing DNA disruption which elicits potent tumor cell killing, bystander activity and immunogenic cell death.

In some embodiments, an antibody conjugate of the present disclosure can include (a) an antibody comprising: (1) a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO; 2512; (2) a V_{H} comprising: a CDR-H1 comprising at least one of SEQ ID NOs: 161 and 486; a CDR-H2 comprising at least one of SEQ ID NOs: 811 and 1136; and a CDR-H3 comprising at least one of SEQ ID NOs: 1461 and 1786; and a V_{L} comprising: a CDR-L1 comprising SEQ ID NO: 2064; a CDR-L2 comprising SEQ ID NO: 2288; and a CDR-L3 comprising SEQ ID NO; 2512; (3) a V_{H} comprising: a CDR-H1 comprising at least one of SEQ ID NOs: 189 and 514; a CDR-H2 comprising at least one of SEQ ID NOs: 839 and 1164; and a CDR-H3 comprising at least one of SEQ ID NOs: 1489 and 1814; and a V_{L} comprising: a CDR-L1 comprising SEQ ID NO: 2064; a CDR-L2 comprising SEQ ID NO: 2288; and a CDR-L3 comprising SEQ ID NO; 2512; (4) a V_{H} comprising: a CDR-H1 comprising at least one of SEQ ID NOs: 192 and 517; a CDR-H2 comprising at least one of SEQ ID NOs: 842 and 1167; and a CDR-H3 comprising at least one of SEQ ID NOs: 1492 and 1817; and a V_{L} comprising: a CDR-L1 comprising SEQ ID NO: 2064; a CDR-L2 comprising SEQ ID NO: 2288; and a CDR-L3 comprising SEQ ID NO; 2512; (5) a V_{H} comprising: a CDR-H1 comprising at least one of SEQ ID NOs: 211 and 536; a CDR-H2 comprising at least one of SEQ ID NOs: 861 and 1186; and a CDR-H3 comprising at least one of SEQ ID NOs: 1511 and 1836; and a V_{L} comprising: a CDR-L1 comprising SEQ ID NO: 2064; a CDR-L2 comprising SEQ ID NO: 2288; and a CDR-L3 comprising SEQ ID NO; 2512; (6) a V_{H} comprising: a CDR-H1 comprising at least one of SEQ ID NOs: 214 and 539; a CDR-H2 comprising at least one of SEQ ID NOs: 864 and 1189; and a CDR-H3 comprising at least one of SEQ ID NOs: 1514 and 1839; and a V_{L} comprising: a CDR-L1 comprising SEQ ID NO: 2064; a CDR-L2 comprising SEQ ID NO: 2288; and a CDR-L3 comprising SEQ ID NO; 2512; (7) a V_{H} comprising: a CDR-H1 comprising at least one of SEQ ID NOs: 218 and 543; a CDR-H2 comprising at least one of SEQ ID NOs: 868 and 1210; and a CDR-H3 comprising at least one of SEQ ID NOs: 1518 and 1843; and a V_{L} comprising: a CDR-L1 comprising at least one of SEQ ID NOs: 1974 and 2086; a CDR-L2 comprising at least one of SEQ ID NOs: 2198 and 2310; and a CDR-L3 comprising at least one of SEQ ID NOs: 2422 and 2534; (8) a V_{H} comprising: a CDR-H1 comprising at least one of SEQ ID NOs: 105 and 430; a CDR-H2 comprising at least one of SEQ ID NOs: 755 and 1080; and a CDR-H3 comprising at least one of SEQ ID NOs: 1405 and 1730; and a V_{L} comprising: a CDR-L1 comprising SEQ ID NO: 2064; a CDR-L2 comprising SEQ ID NO: 2288; and a CDR-L3 comprising SEQ ID NO; 2512; (9) a V_{H} comprising: a CDR-H1 comprising at least one of SEQ ID NOs: 114 and 439; a CDR-H2 comprising at least one of SEQ ID NOs: 764 and 1089; and a CDR-H3 comprising at least one of SEQ ID NOs: 1414 and 1739; and a V_{L} comprising: a CDR-L1 comprising SEQ ID NO: 2064; a CDR-L2 comprising SEQ ID NO: 2288; and a CDR-L3 comprising SEQ ID NO; 2512; (10) a V_{H} comprising: a CDR-H1 comprising at least one of SEQ ID NOs: 117 and 442; a CDR-H2 comprising at least one of SEQ ID NOs: 767 and 1092; and a CDR-H3 comprising at least one of SEQ ID NOs: 1417 and 1742; and a V_{L} comprising: a CDR-L1 comprising SEQ ID NO: 2064; a CDR-L2 comprising SEQ ID NO: 2288; and a CDR-L3 comprising SEQ ID NO; 2512; (b) para-azidomethylphenylalanine residues at antibody sites selected from the group consisting of HC180, HC241, HC404, LC42, LC161 and combinations thereof; and (c) a linker-payload selected from the group consisting of:

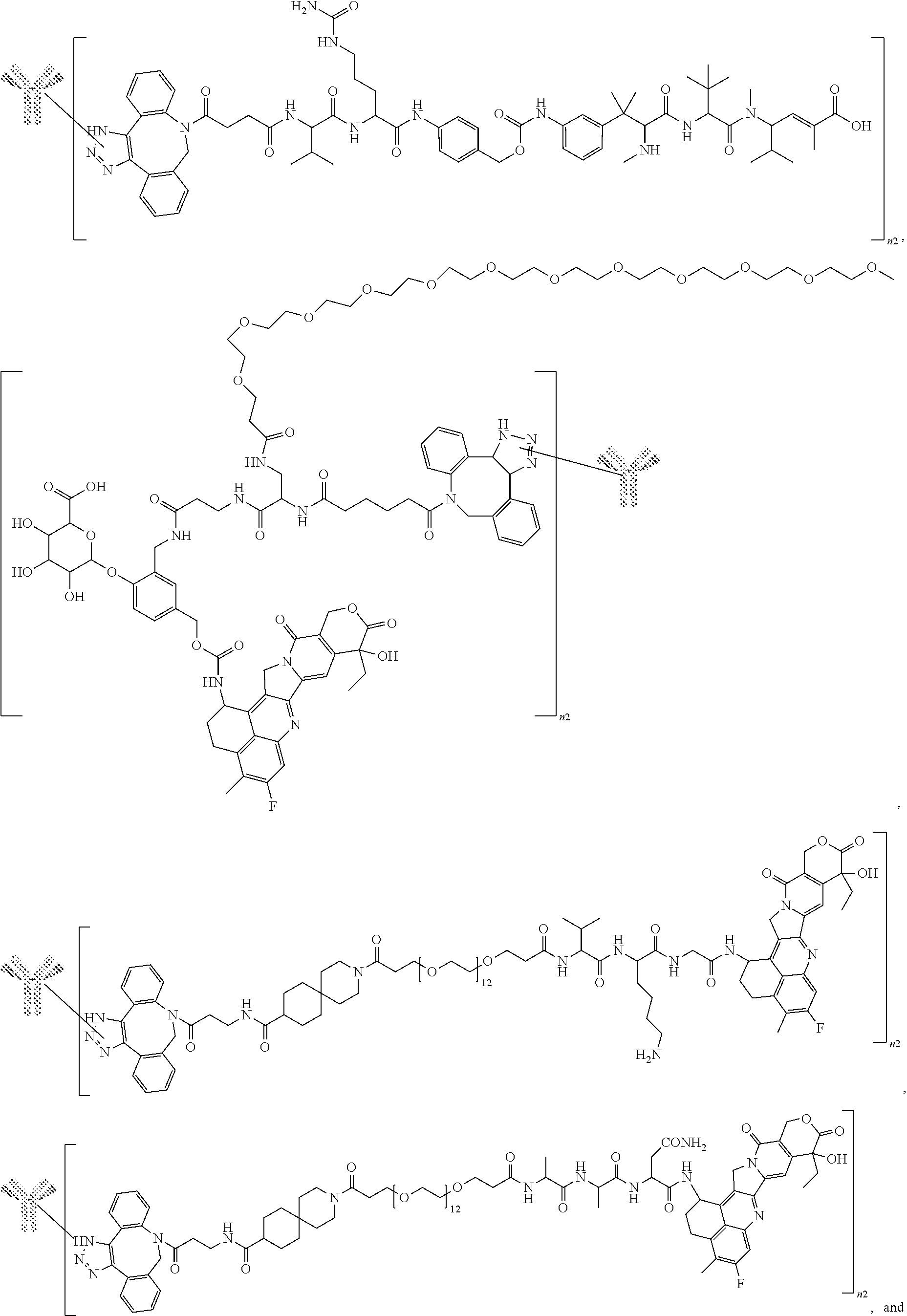

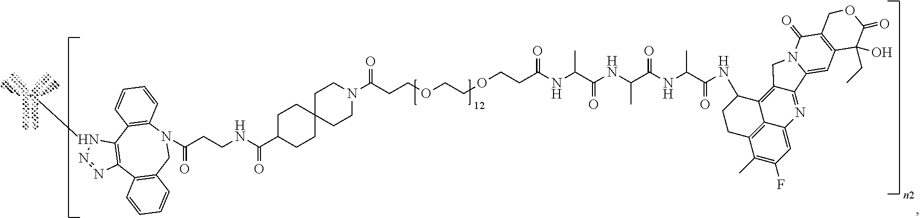

wherein n2 is 2, 4, 6, 8 or 10 and each linker-payload is bonded to one of the para-azidomethylphenylalanine residue side chains.

In some embodiments, an antibody conjugate of the present disclosure can include (a) an antibody comprising: (1) a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO; 2512; (2) a V_{H} comprising: a CDR-H1 comprising at least one of SEQ ID NOs: 161 and 486; a CDR-H2 comprising at least one of SEQ ID NOs: 811 and 1136; and a CDR-H3 comprising at least one of SEQ ID NOs: 1461 and 1786; and a V_{L} comprising: a CDR-L1 comprising SEQ ID NO: 2064; a CDR-L2 comprising SEQ ID NO: 2288; and a CDR-L3 comprising SEQ ID NO; 2512; (3) a V_{H} comprising: a CDR-H1 comprising at least one of SEQ ID NOs: 189 and 514; a CDR-H2 comprising at least one of SEQ ID NOs: 839 and 1164; and a CDR-H3 comprising at least one of SEQ ID NOs: 1489 and 1814; and a V_{L} comprising: a CDR-L1 comprising SEQ ID NO: 2064; a CDR-L2 comprising SEQ ID NO: 2288; and a CDR-L3 comprising SEQ ID NO; 2512; (4) a V_{H} comprising: a CDR-H1 comprising at least one of SEQ ID NOs: 192 and 517; a CDR-H2 comprising at least one of SEQ ID NOs: 842 and 1167; and a CDR-H3 comprising at least one of SEQ ID NOs: 1492 and 1817; and a V_{L} comprising: a CDR-L1 comprising SEQ ID NO: 2064; a CDR-L2 comprising SEQ ID NO: 2288; and a CDR-L3 comprising SEQ ID NO; 2512; (5) a V_{H} comprising: a CDR-H1 comprising at least one of SEQ ID NOs: 211 and 536; a CDR-H2 comprising at least one of SEQ ID NOs: 861 and 1186; and a CDR-H3 comprising at least one of SEQ ID NOs: 1511 and 1836; and a V_{L} comprising: a CDR-L1 comprising SEQ ID NO: 2064; a CDR-L2 comprising SEQ ID NO: 2288; and a CDR-L3 comprising SEQ ID NO; 2512; (6) a V_{H} comprising: a CDR-H1 comprising at least one of SEQ ID NOs: 214 and 539; a CDR-H2 comprising at least one of SEQ ID NOs: 864 and 1189; and a CDR-H3 comprising at least one of SEQ ID NOs: 1514 and 1839; and a V_{L} comprising: a CDR-L1 comprising SEQ ID NO: 2064; a CDR-L2 comprising SEQ ID NO: 2288; and a CDR-L3 comprising SEQ ID NO; 2512; (7) a V_{H} comprising: a CDR-H1 comprising at least one of SEQ ID NOs: 218 and 543; a CDR-H2 comprising at least one of SEQ ID NOs: 868 and 1210; and a CDR-H3 comprising at least one of SEQ ID NOs: 1518 and 1843; and a V_{L} comprising: a CDR-L1 comprising at least one of SEQ ID NOs: 1974 and 2086; a CDR-L2 comprising at least one of SEQ ID NOs: 2198 and 2310; and a CDR-L3 comprising at least one of SEQ ID NOs: 2422 and 2534; (8) a V_{H} comprising: a CDR-H1 comprising at least one of SEQ ID NOs: 105 and 430; a CDR-H2 comprising at least one of SEQ ID NOs: 755 and 1080; and a CDR-H3 comprising at least one of SEQ ID NOs: 1405 and 1730; and a V_{L} comprising: a CDR-L1 comprising SEQ ID NO: 2064; a CDR-L2 comprising SEQ ID NO: 2288; and a CDR-L3 comprising SEQ ID NO; 2512; (9) a V_{H} comprising: a CDR-H1 comprising at least one of SEQ ID NOs: 114 and 439; a CDR-H2 comprising at least one of SEQ ID NOs: 764 and 1089; and a CDR-H3 comprising at least one of SEQ ID NOs: 1414 and 1739; and a V_{L} comprising: a CDR-L1 comprising SEQ ID NO: 2064; a CDR-L2 comprising SEQ ID NO: 2288; and a CDR-L3 comprising SEQ ID NO; 2512; (10) a V_{H} comprising: a CDR-H1 comprising at least one of SEQ ID NOs: 117 and 442; a CDR-H2 comprising at least one of SEQ ID NOs: 767 and 1092; and a CDR-H3 comprising at least one of SEQ ID NOs: 1417 and 1742; and a V_{L} comprising: a CDR-L1 comprising SEQ ID NO: 2064; a CDR-L2 comprising SEQ ID NO: 2288; and a CDR-L3 comprising SEQ ID NO; 2512; (b) para-azidomethylphenylalanine residues at antibody sites selected from the group consisting of HC180, HC241, HC404, LC42, LC161 and combinations thereof; and (c) a linker-payload selected from the group consisting of:

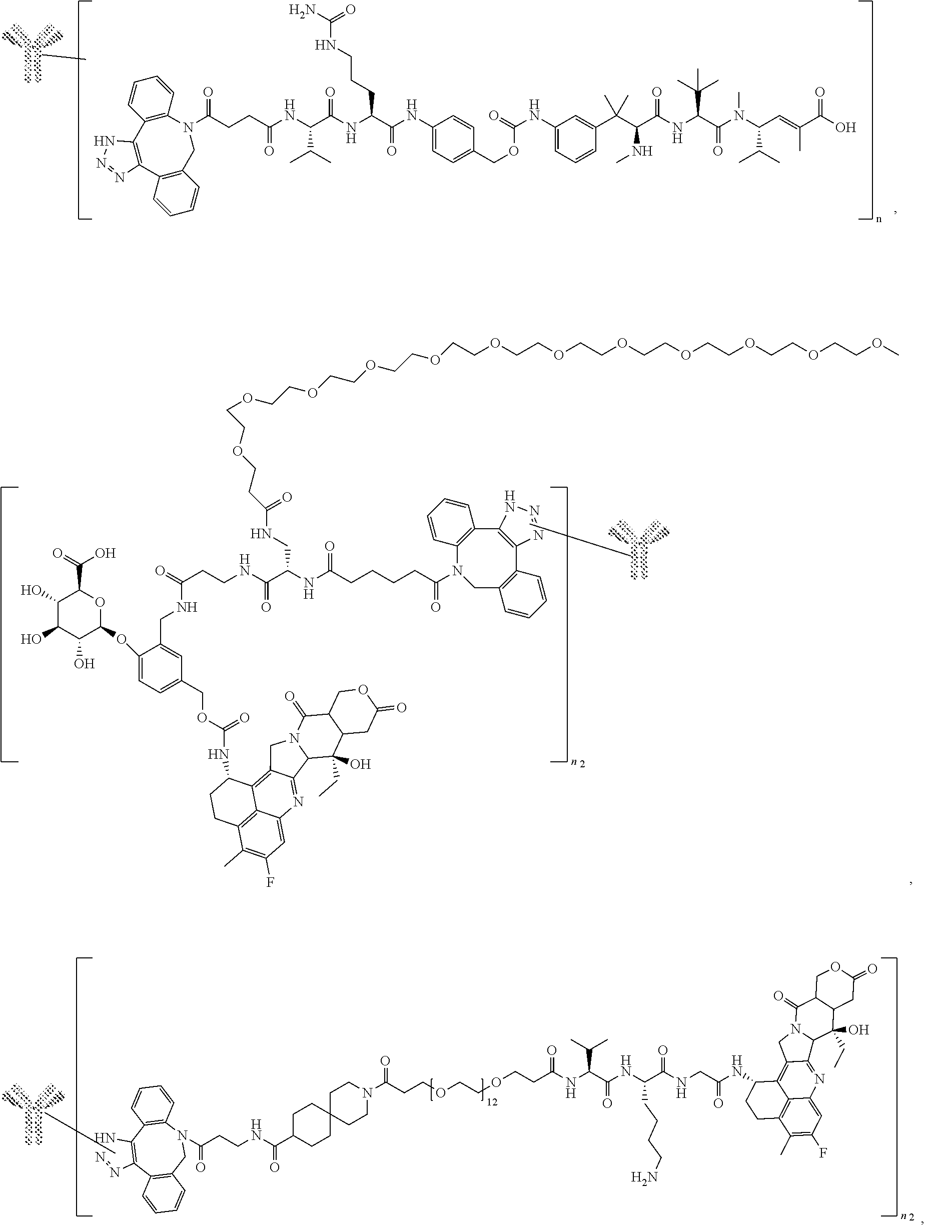

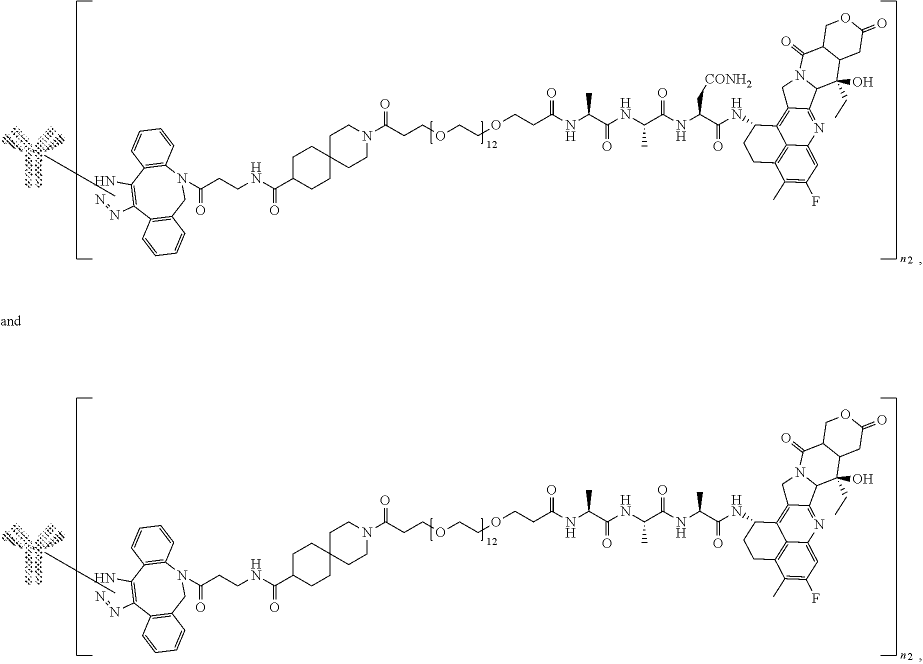

wherein n2 is 2, 4, 6, 8 or 10 and each linker-payload is bonded to one of the para-azidomethylphenylalanine residue side chains.

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites selected from the group consisting of HC180, HC241, HC404, LC42, LC161 and combinations thereof; and (c) a linker-payload selected from the group consisting of:

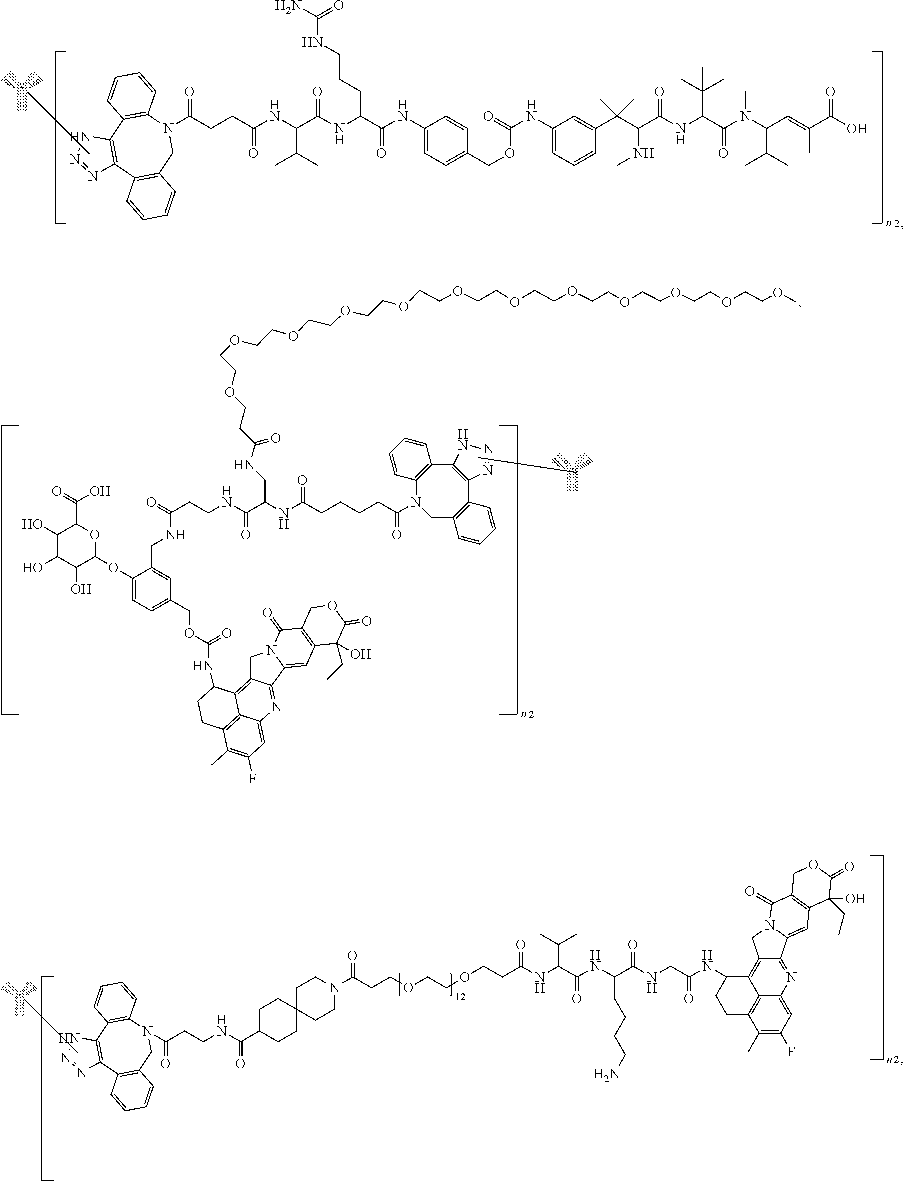

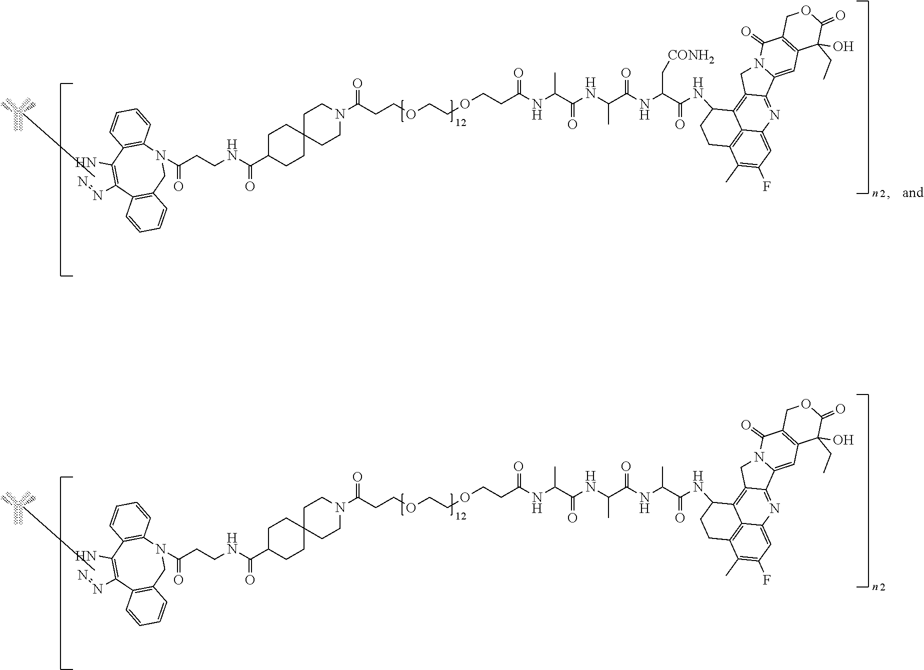

wherein n2 is 2, 4, 6, 8 or 10 and each linker-payload is bonded to one of the para-azidomethylphenylalanine residue side chains.

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites selected from the group consisting of HC180, HC241, HC404, LC42, LC161 and combinations thereof; and (c) a linker-payload selected from the group consisting of:

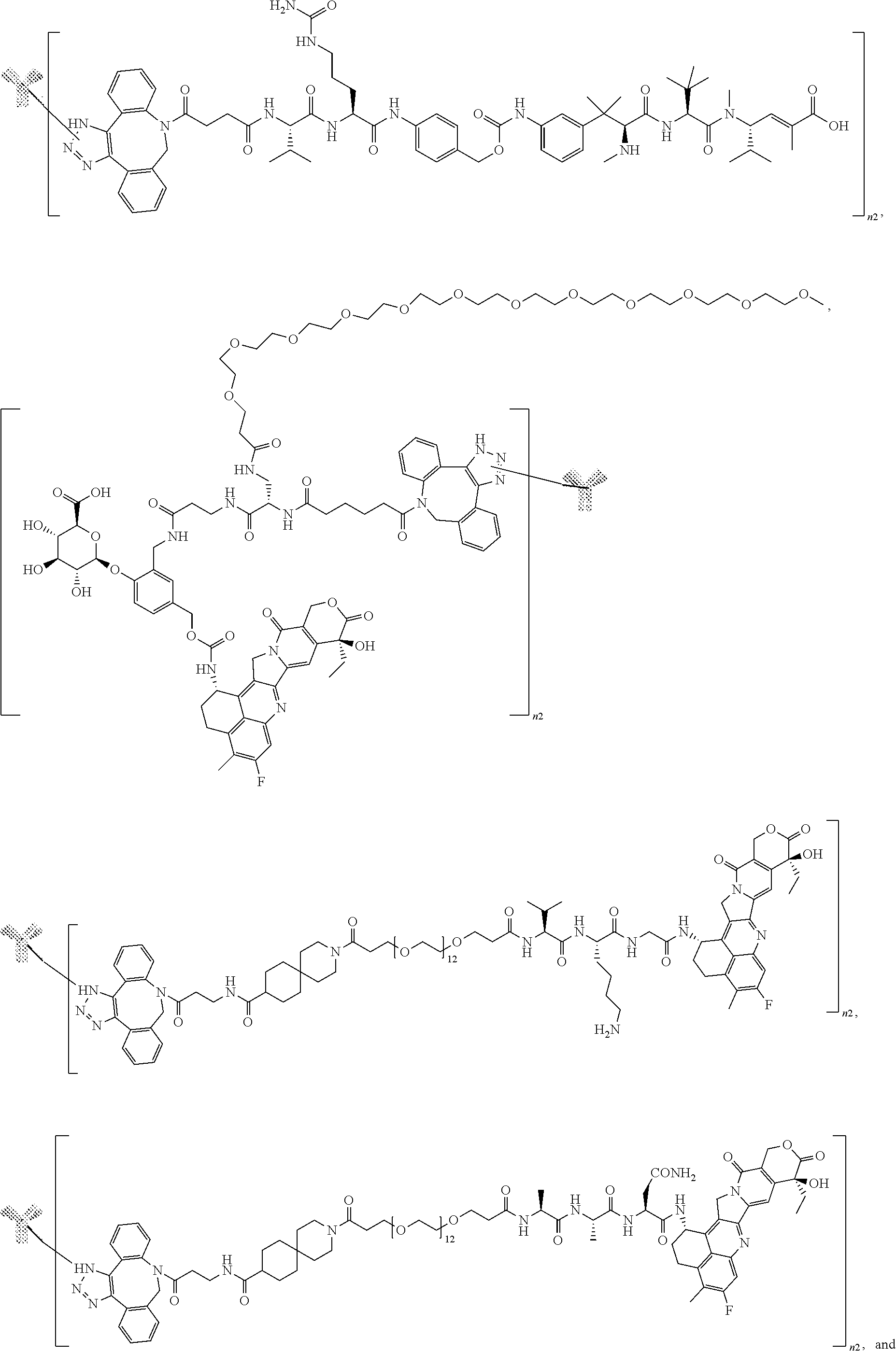

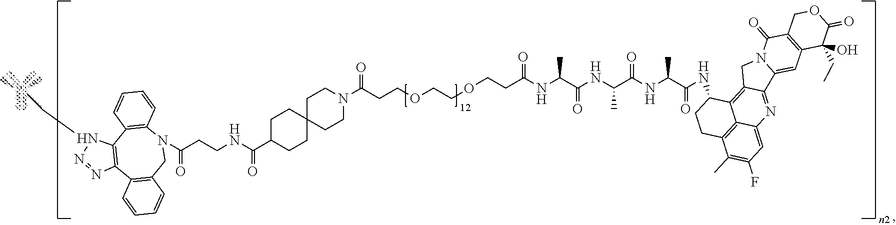

wherein n2 is 2, 4, 6, 8 or 10 and each linker-payload is bonded to one of the para-azidomethylphenylalanine residue side chains.

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC241 and HC404; and (c) a linker-payload, wherein n2 is 4 and each linker-payload is bonded to one of the para-azidomethylphenylalanine residue side chains:

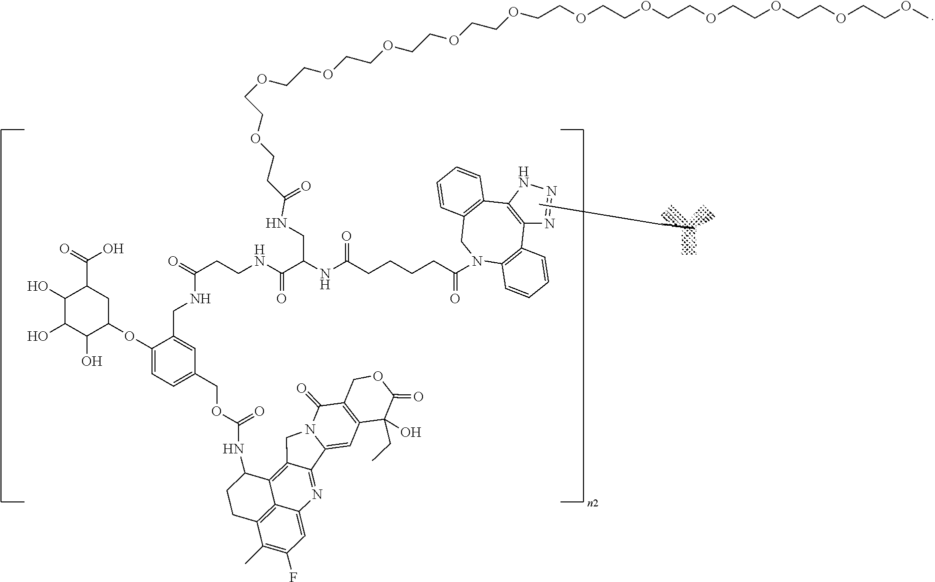

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC241 and HC404; and (c) a linker-payload, wherein n2 is 4 and each linker-payload is bonded to one of the para-azidomethylphenylalanine residue side chains:

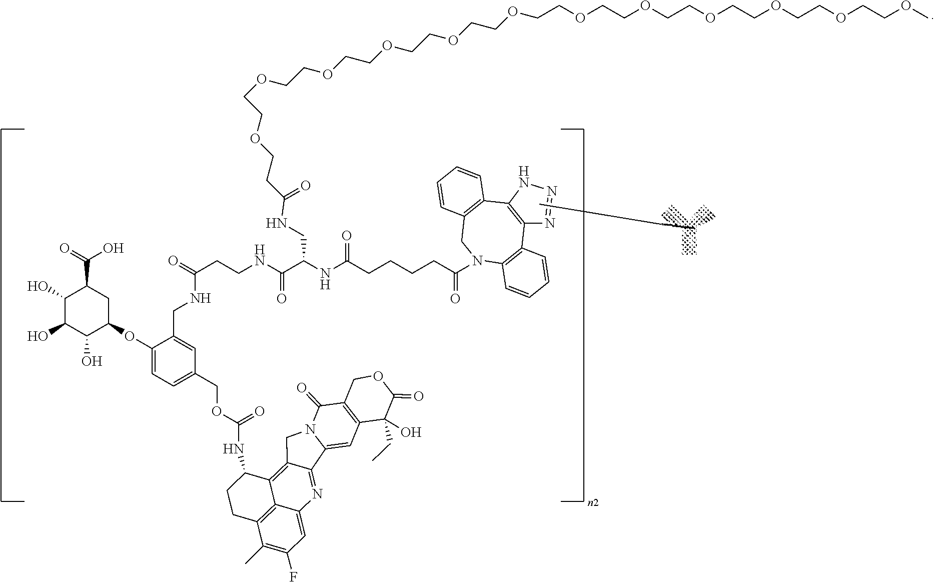

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC241, HC404, HC180, and HC391; and (c) a linker-payload, wherein n2 is 8 and each linker-payload is bonded to one of the para-azidomethylphenylalanine residue side chains:

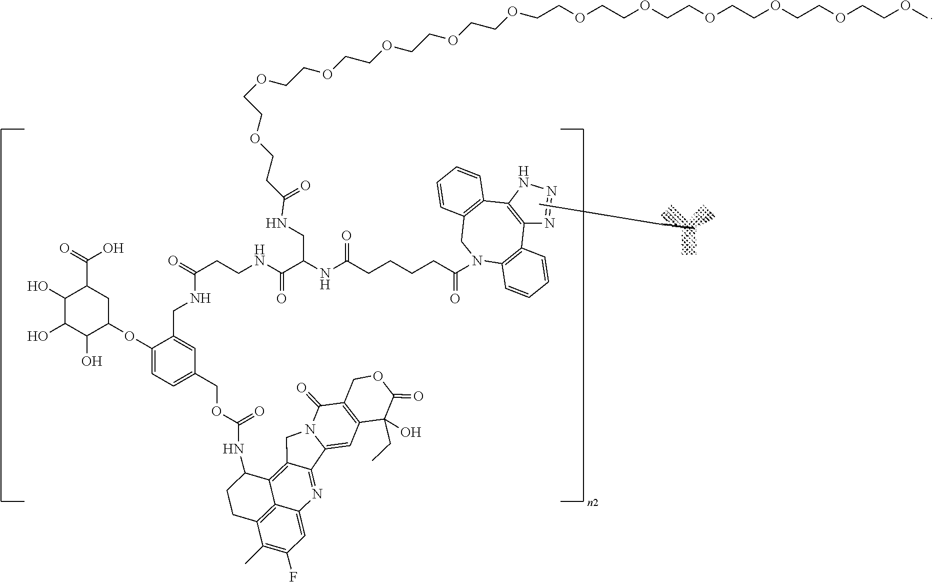

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the

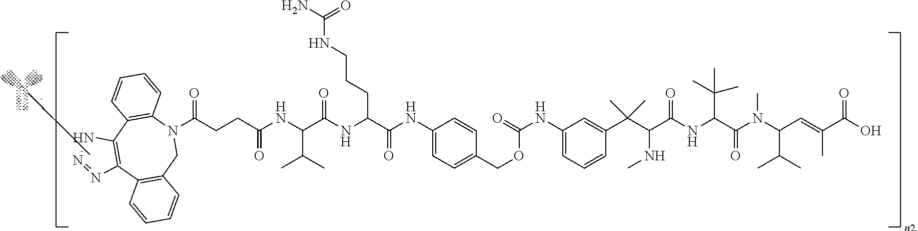

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

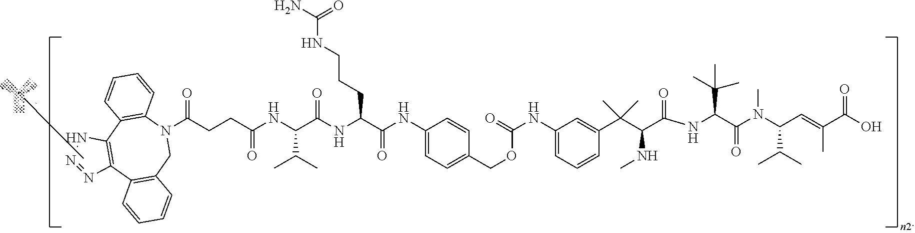

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC241 and HC404; and (c) a linker-payload, wherein n2 is 4 and each linker-payload is bonded to one of the para-azidomethylphenylalanine residue side chains:

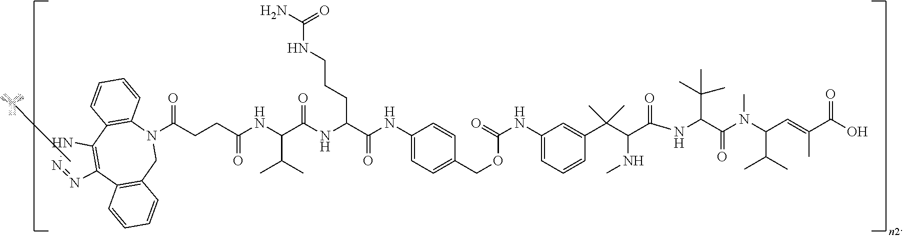

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC241 and HC404; and (c) a linker-payload, wherein n2 is 4 and each linker-payload is bonded to one of the para-azidomethylphenylalanine residue side chains:

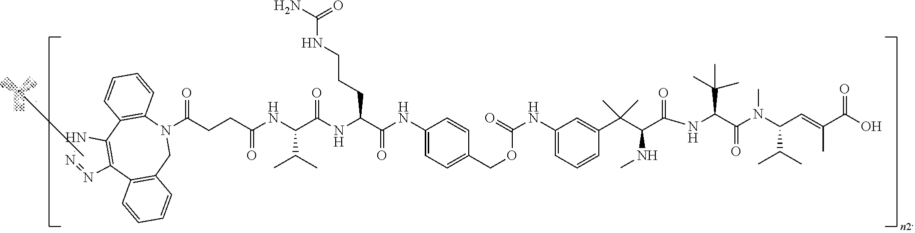

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180, HC404 and LC42; and (c) a linker-payload, wherein n2 is 6 and each linker-payload is bonded to one of the para-azidomethylphenylalanine residue side chains:

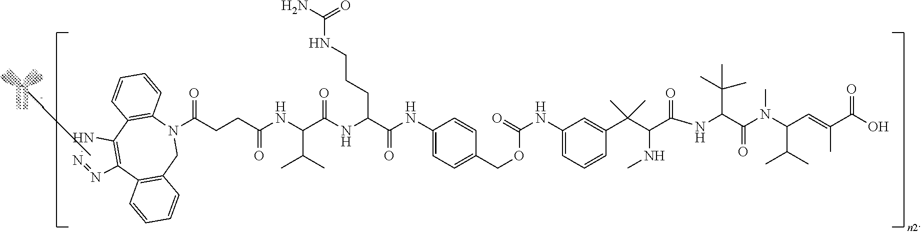

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180, HC404 and LC42; and (c) a linker-payload, wherein n2 is 6 and each linker-payload is bonded to one of the para-azidomethylphenylalanine residue side chains:

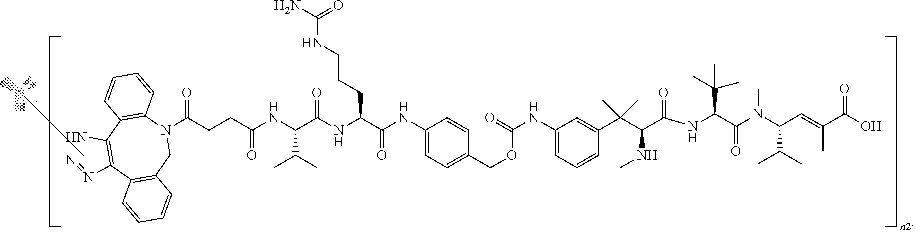

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180, HC241 and HC404; and (c) a linker-payload, wherein n2 is 6 and each linker-payload is bonded to one of the para-azidomethylphenylalanine residue chains:

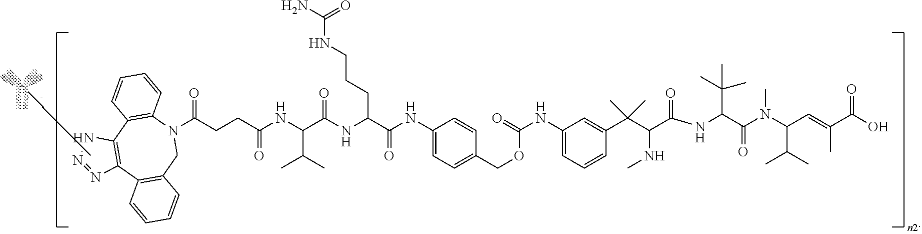

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180, HC241 and HC404; and (c) a linker-payload, wherein n2 is 6 and each linker-payload is bonded to one of the para-azidomethylphenylalanine residue side chains:

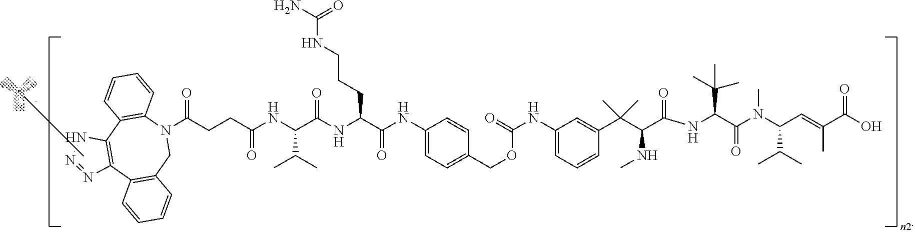

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180, HC404, LC42 and LC161; and (c) a linker-payload, wherein n2 is 8 and each linker-payload is bonded to one of the para-azidomethylphenylalanine residue side chains:

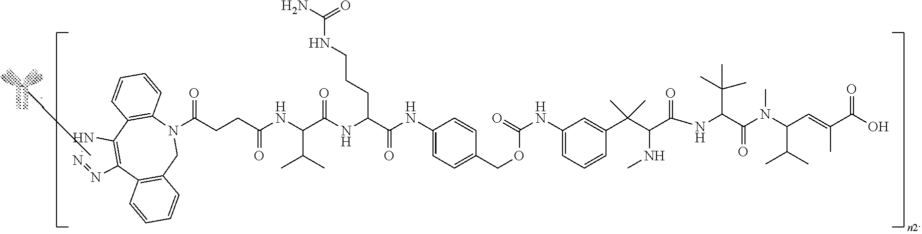

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180, HC404, LC42 and LC161; and (c) a linker-payload, wherein n2 is 8 and each linker-payload is bonded to one of the para-azidomethylphenylalanine residue side chains:

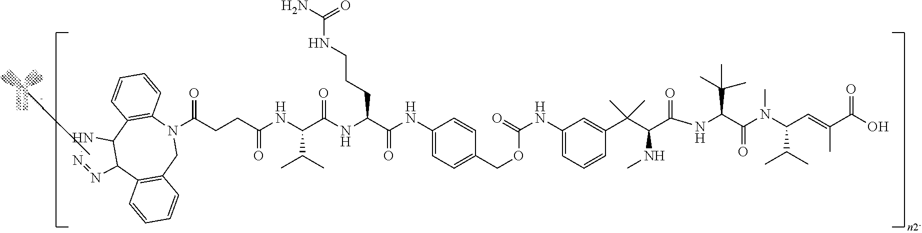

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180, HC241, HC404 and LC42; and (c) a linker-payload, wherein n2 is 8 and each linker-payload is bonded to one of the para-azidomethylphenylalanine residue side chains:

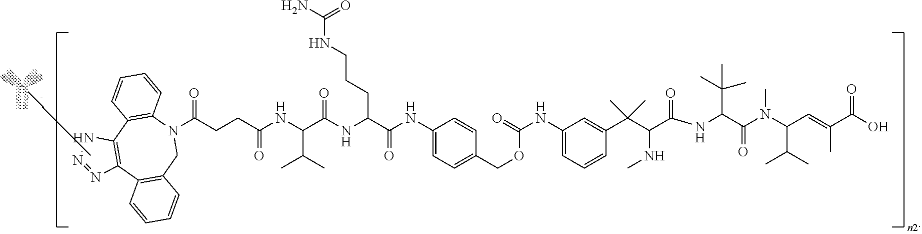

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180, HC241, HC404 and LC42; and (c) a linker-payload, wherein n2 is 8 and each linker-payload is bonded to one of the para-azidomethylphenylalanine residue side chains:

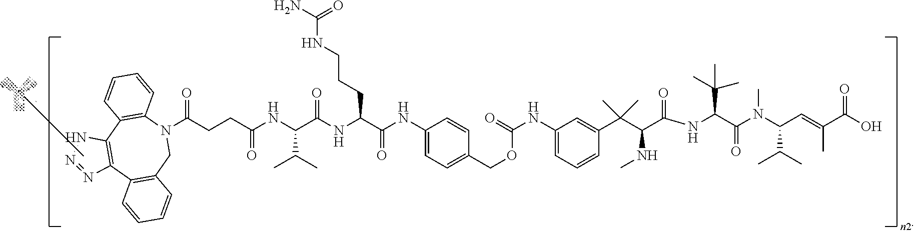

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180, HC241, HC404 and LC42; and (c) a linker-payload, wherein n2 is 8 and each linker-payload is bonded to one of the para-azidomethylphenylalanine residue side chains:

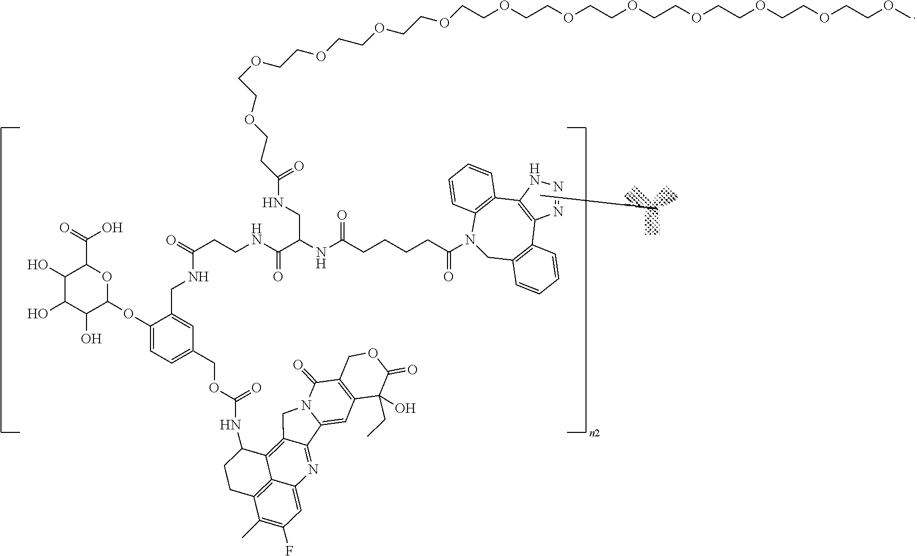

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180, HC241, HC404 and LC42; and (c) a linker-payload, wherein n2 is 8 and each linker-payload is bonded to one of the para-azidomethylphenylalanine residue side chains:

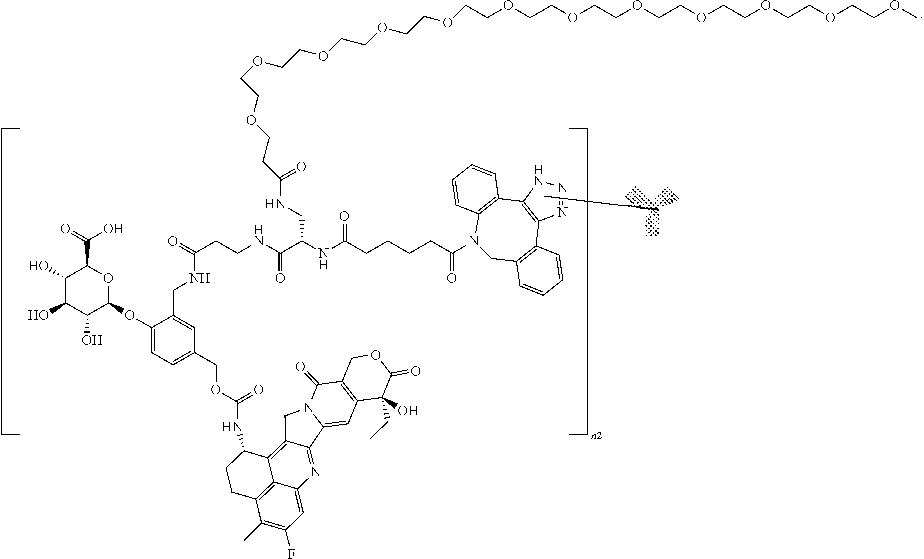

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

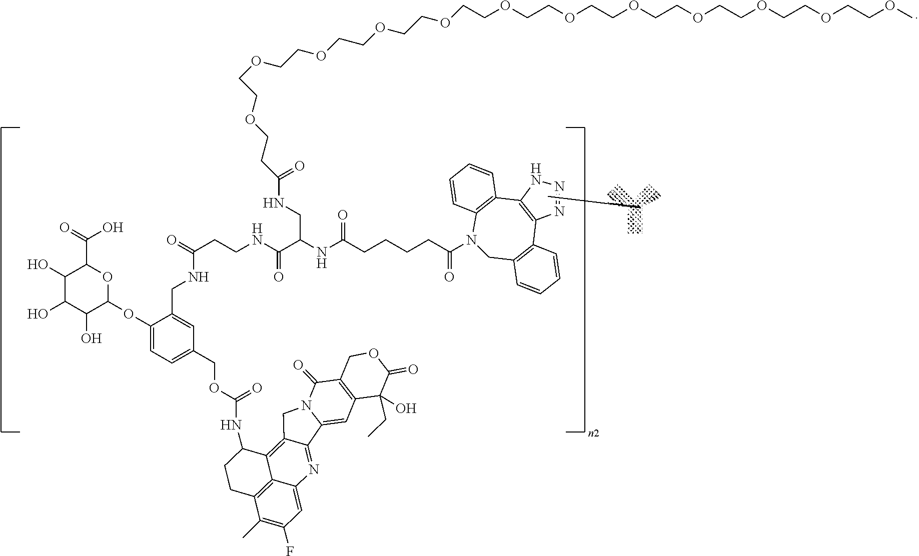

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

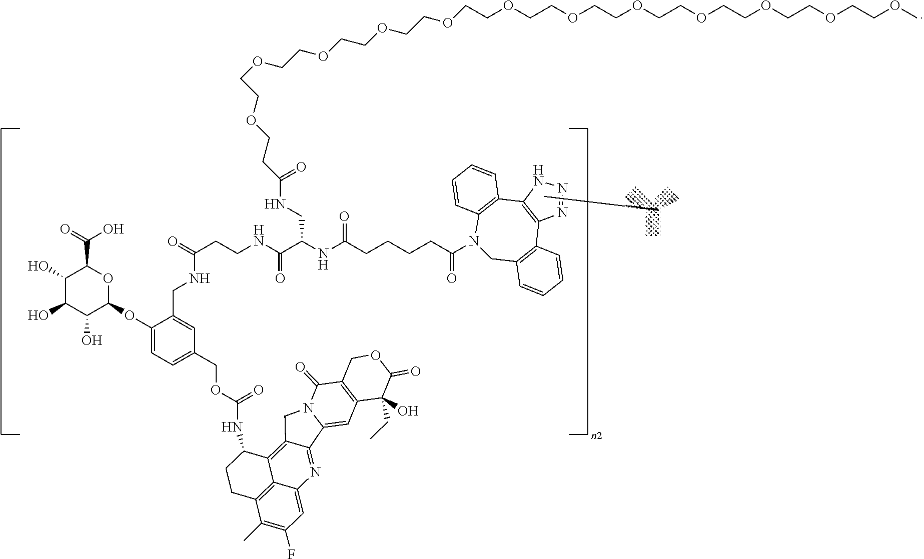

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

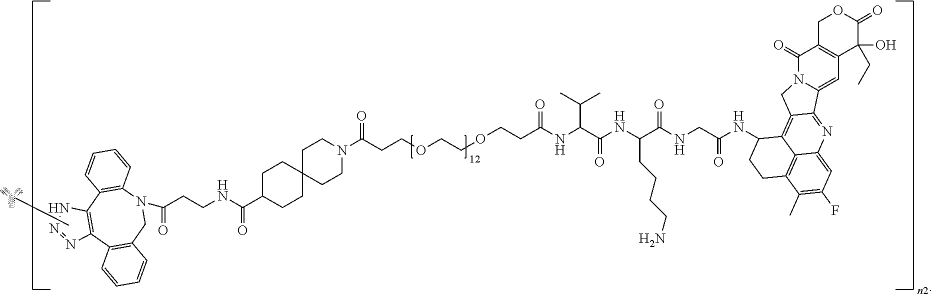

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

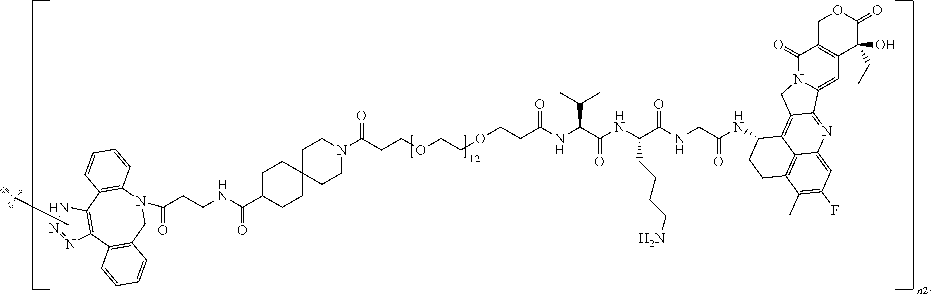

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

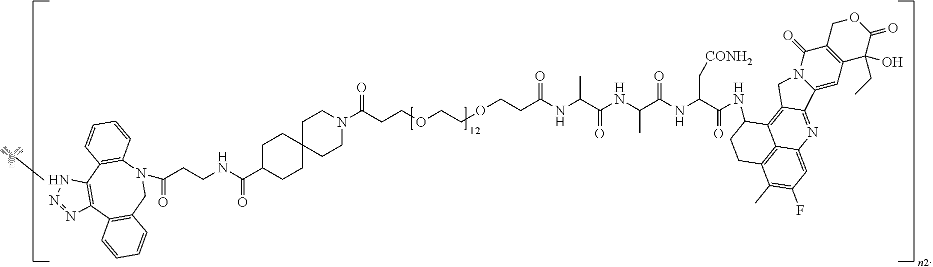

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

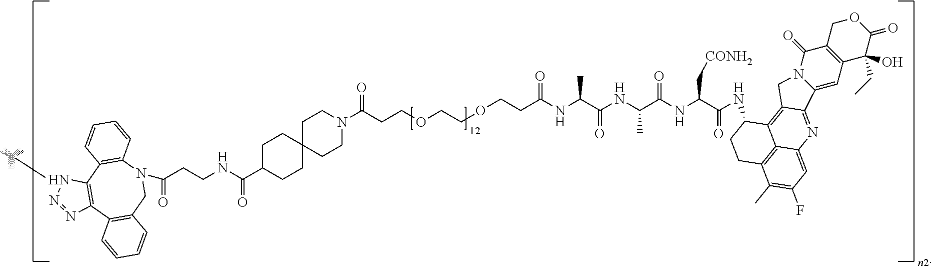

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

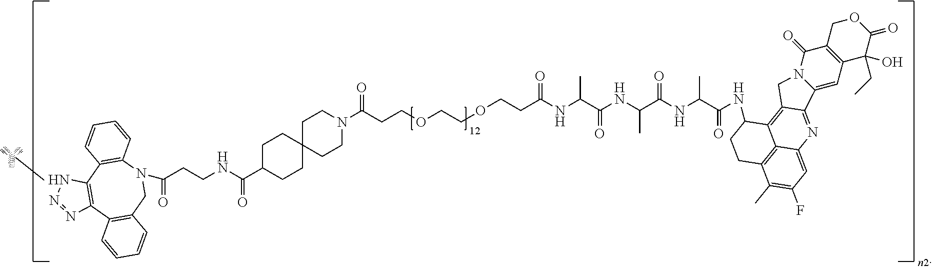

In some embodiments, an antibody conjugate is provided that comprises: (a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

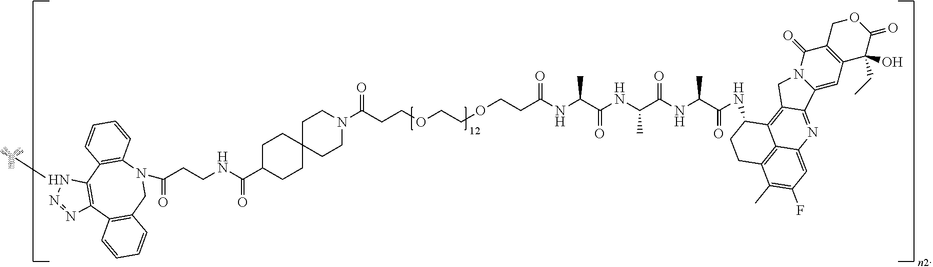

In some embodiments, the antibody of the antibody drug conjugate comprises the V_{H}/V_{L} pair SEQ ID NOs: 2752/3061.

In some embodiments, the antibody of the antibody drug conjugate comprises the complementarity determining regions (CDRs) of a V_{H} region selected from SEQ ID NOs: 2727, 2736, 2739, 2751-2838, 2840, 2846, 2851, 2857, and 2859-2860 and the CDRs of a V_{L} region selected from SEQ ID NOs: 2951, 2957, 2962, 2968, 2970-2971, and 3061. In some embodiments, the antibody of the antibody conjugate comprises a V_{H} region selected from SEQ ID NOs: 2727, 2736, 2739, 2751-2838, 2840, 2846, 2851, 2857, and 2859-2860 and a V_{L} region selected from SEQ ID NOs: 2951, 2957, 2962, 2968, 2970-2971, and 3061. In some embodiments, the antibody of the antibody conjugate binds to the same epitope as a second antibody with a V_{H} region selected from SEQ ID NOs: 2727, 2736, 2739, 2751-2838, 2840, 2846, 2851, 2857, and 2859-2860 and a V_{L} region selected from SEQ ID NOs: 2951, 2957, 2962, 2968, 2970-2971, and 3061.

In some embodiments, an antibody conjugate is provided that comprises: (a) an antibody comprising a V_{H} comprising: a CDR-H1 comprising at least one of SEQ ID NOs: 161 and 486; a CDR-H2 comprising at least one of SEQ ID NOs: 811 and 1136; and a CDR-H3 comprising at least one of SEQ ID NOs: 1461 and 1786; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

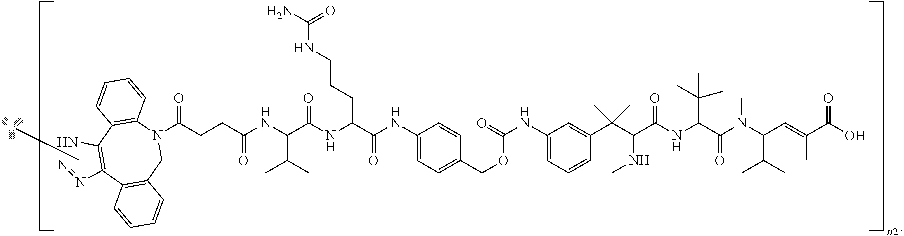

In some embodiments, the antibody of the antibody conjugate comprises the V_{H}/V_{L} pair SEQ ID NOs: 2783/3061.

In some embodiments, an antibody conjugate is provided that comprises: (a) an antibody comprising a V_{H} comprising: a CDR-H1 comprising at least one of SEQ ID NOs: 161 and 486; a CDR-H2 comprising at least one of SEQ ID NOs: 811 and 1136; and a CDR-H3 comprising at least one of SEQ ID NOs: 1461 and 1786; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of chains: the para-azidomethylphenylalanine residue side

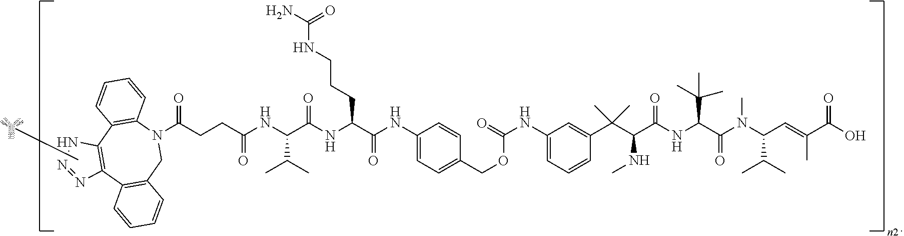

In some embodiments, the antibody of the antibody conjugate comprises the V_{H}/V_{L} pair SEQ ID NOs: 2783/3061.

In some embodiments, an antibody conjugate is provided that comprises: (a) an antibody comprising a V_{H} comprising: a CDR-H1 comprising at least one of SEQ ID NOs: 189 and 514; a CDR-H2 comprising at least one of SEQ ID NOs: 839 and 1164; and a CDR-H3 comprising at least one of SEQ ID NOs: 1489 and 1814; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

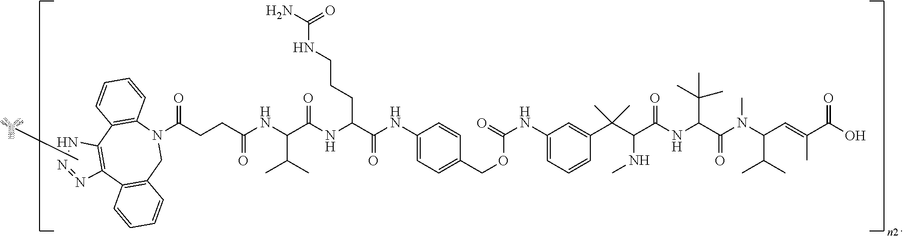

In some embodiments, the antibody of the antibody conjugate comprises the V_{H}/V_{L} pair SEQ ID NOs: 2811/3061.

In some embodiments, an antibody conjugate is provided that comprises: (a) an antibody comprising a V_{H} comprising: a CDR-H1 comprising at least one of SEQ ID NOs: 189 and 514; a CDR-H2 comprising at least one of SEQ ID NOs: 839 and 1164; and a CDR-H3 comprising at least one of SEQ ID NOs: 1489 and 1814; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

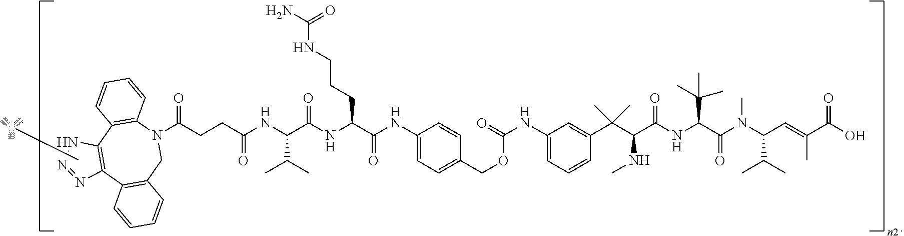

In some embodiments, the antibody of the antibody conjugate comprises the V_{H}/V_{L} pair SEQ ID NOs: 2811/3061.

In some embodiments, an antibody conjugate is provided that comprises: (a) an antibody comprising a V_{H} comprising: a CDR-H1 comprising at least one of SEQ ID NOs: 192 and 517; a CDR-H2 comprising at least one of SEQ ID NOs: 842 and 1167; and a CDR-H3 comprising at least one of SEQ ID NOs: 1492 and 1817; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

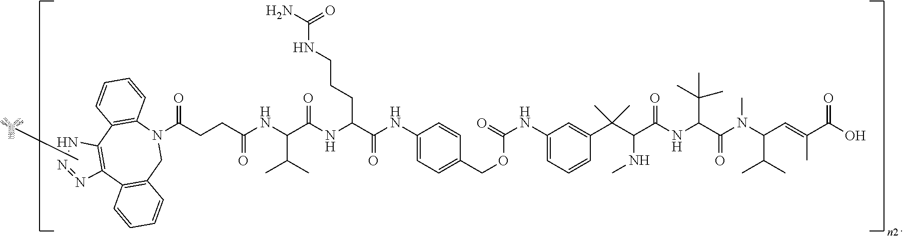

In some embodiments, the antibody of the antibody conjugate comprises the V_{H}/V_{L} pair SEQ ID NOs: 2814/3061.

In some embodiments, an antibody conjugate is provided that comprises: (a) an antibody comprising a V_{H} comprising: a CDR-H1 comprising at least one of SEQ ID NOs: 192 and 517; a CDR-H2 comprising at least one of SEQ ID NOs: 842 and 1167; and a CDR-H3 comprising at least one of SEQ ID NOs: 1492 and 1817; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

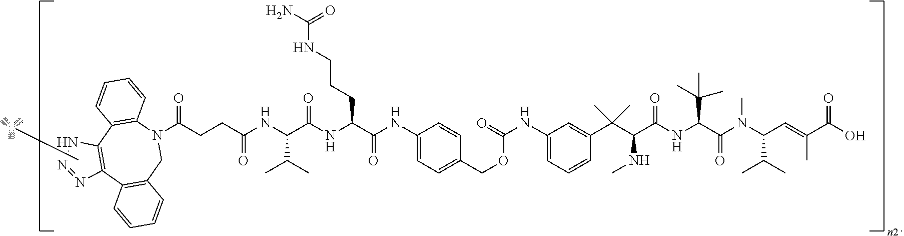

In some embodiments, the antibody of the antibody conjugate comprises the V_{H}/V_{L} pair SEQ ID NOs: 2814/3061.

In some embodiments, an antibody conjugate is provided that comprises: (a) an antibody comprising a V_{H} comprising: a CDR-H1 comprising at least one of SEQ ID NOs: 211 and 536; a CDR-H2 comprising at least one of SEQ ID NOs: 861 and 1186; and a CDR-H3 comprising at least one of SEQ ID NOs: 1511 and 1836; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

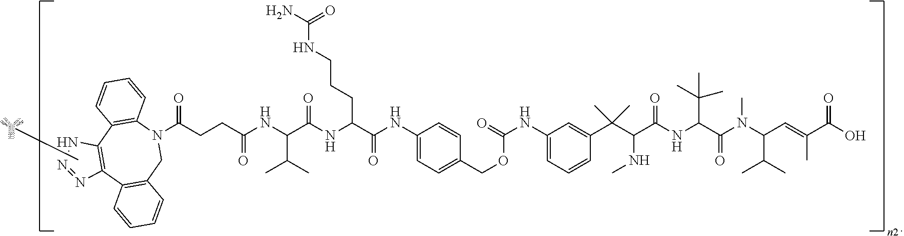

In some embodiments, the antibody of the antibody conjugate comprises the VH/V_{L} pair SEQ ID NOs: 2833/3061.

In some embodiments, an antibody conjugate is provided that comprises: (a) an antibody comprising a V_{H} comprising: a CDR-H1 comprising at least one of SEQ ID NOs: 211 and 536; a CDR-H2 comprising at least one of SEQ ID NOs: 861 and 1186; and a CDR-H3 comprising at least one of SEQ ID NOs: 1511 and 1836; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

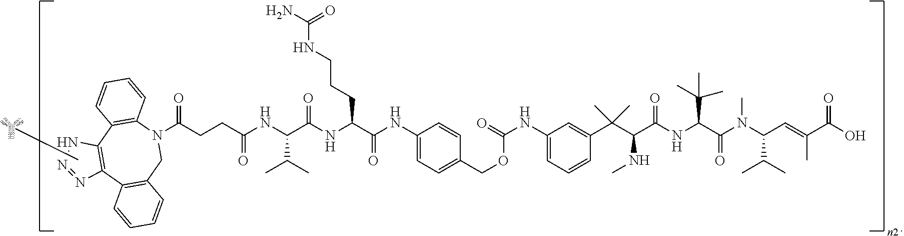

In some embodiments, the antibody of the antibody conjugate comprises the V_{H}/V_{L} pair SEQ ID NOS: 2833/3061.

In some embodiments, an antibody conjugate is provided that comprises: (a) an antibody comprising a V_{H} comprising: a CDR-H1 comprising at least one of SEQ ID NOs: 214 and 539; a CDR-H2 comprising at least one of SEQ ID NOs: 864 and 1189; and a CDR-H3 comprising at least one of SEQ ID NOs: 1514 and 1839; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

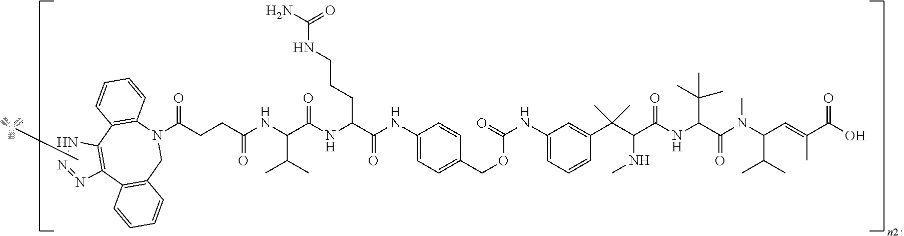

In some embodiments, the antibody of the antibody conjugate comprises the V_{H}/V_{L} pair SEQ ID NOs: 2836/3061.

In some embodiments, an antibody conjugate is provided that comprises (a) an antibody comprising a V_{H} comprising: a CDR-H1 comprising at least one of SEQ ID NOs: 214 and 539; a CDR-H2 comprising at least one of SEQ ID NOs: 864 and 1189; and a CDR-H3 comprising at least one of SEQ ID NOs: 1514 and 1839; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

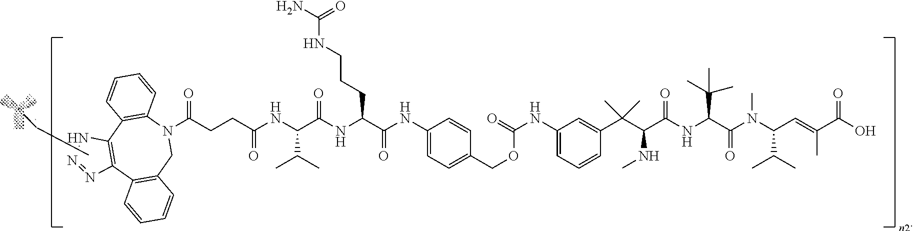

In some embodiments, the antibody of the antibody conjugate comprises the V_{H}/V_{L} pair SEQ ID NOS: 2836/3061.

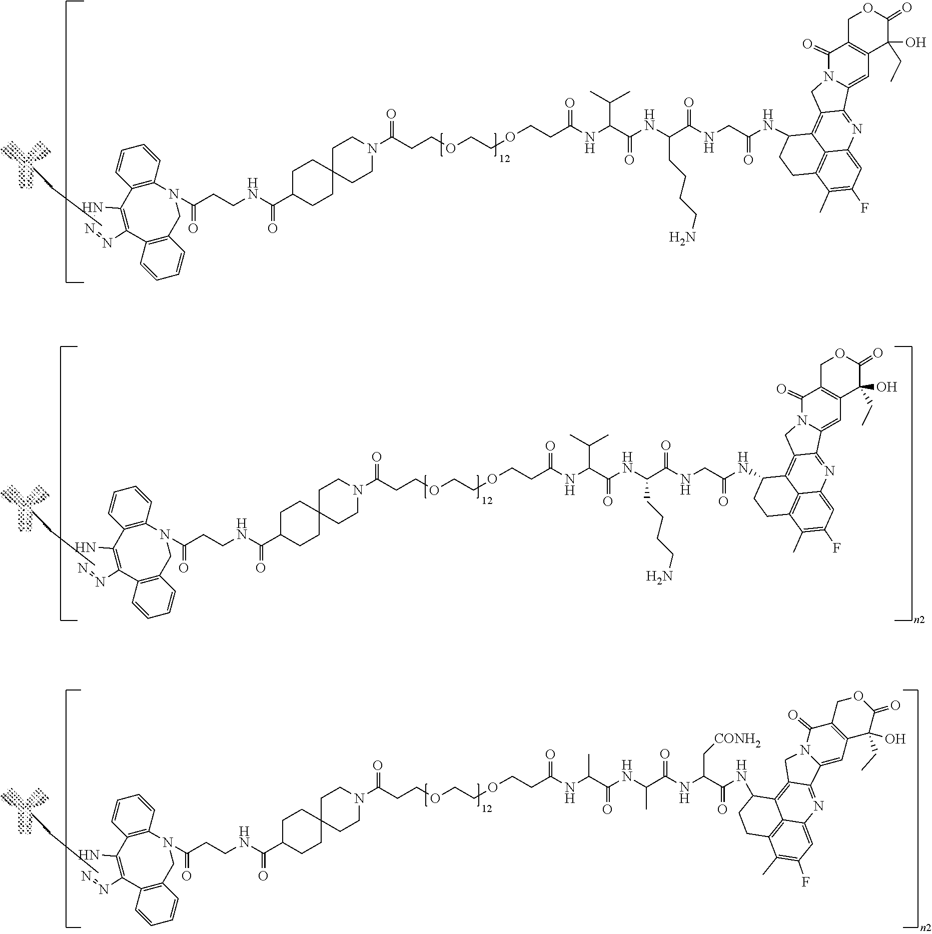

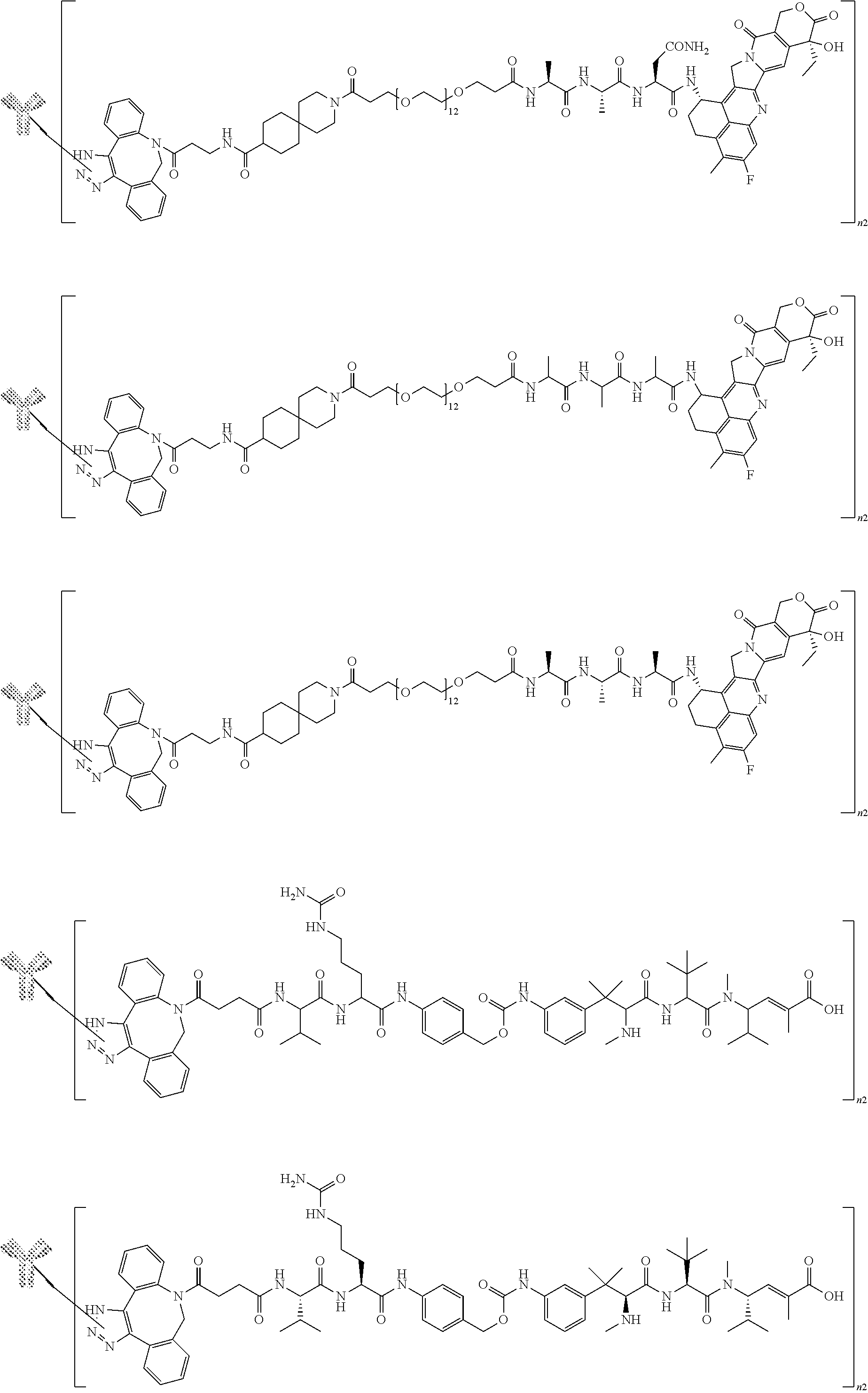

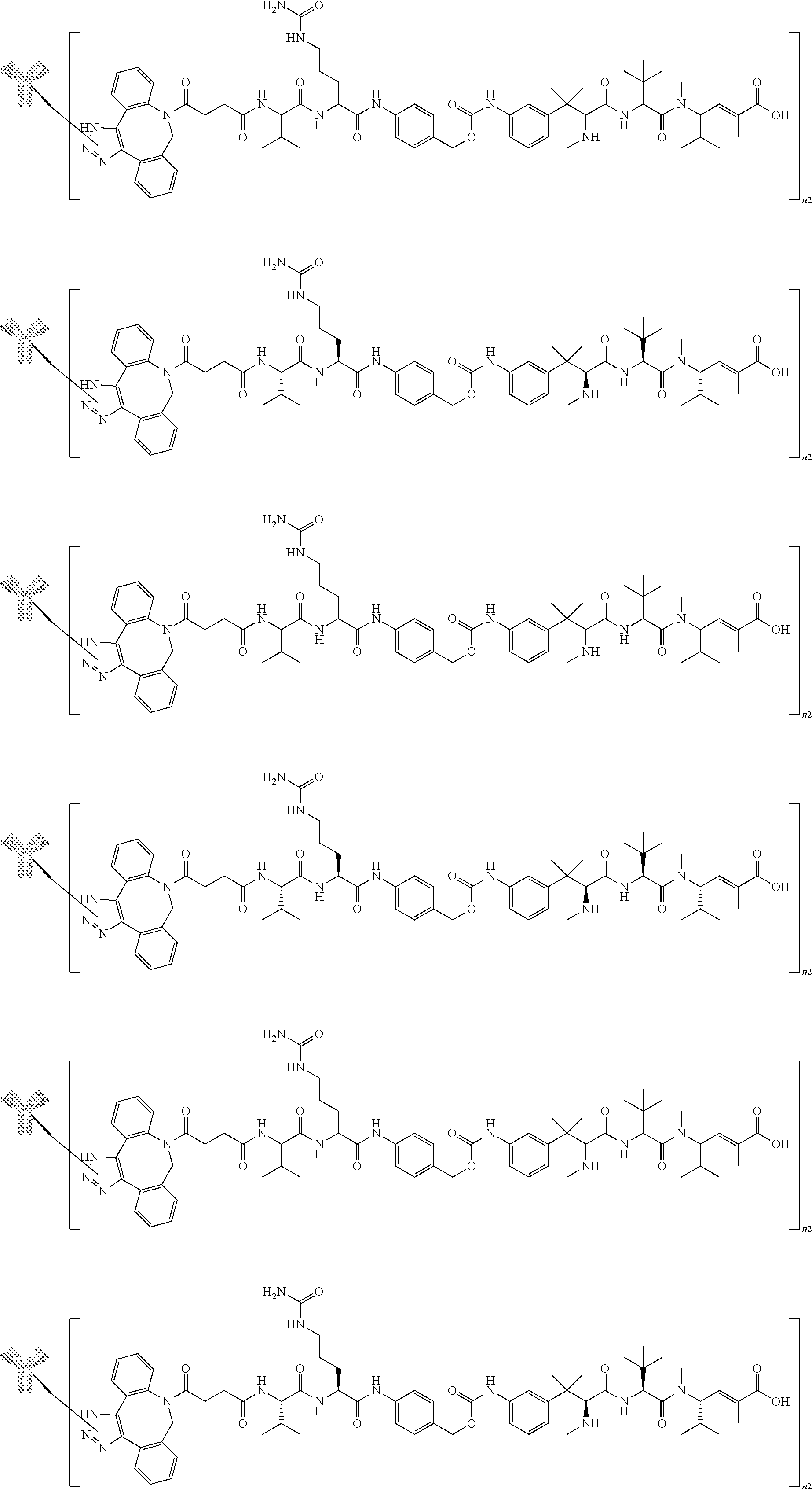

In some embodiments, an antibody conjugate is provided that comprises: (a) an antibody comprising a V_{H} comprising: a CDR-H1 comprising at least one of SEQ ID NOs: 105 and 430; a CDR-H2 comprising at least one of SEQ ID NOs: 755 and 1080; and a CDR-H3 comprising at least one of SEQ ID NOs: 1405 and 1730; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

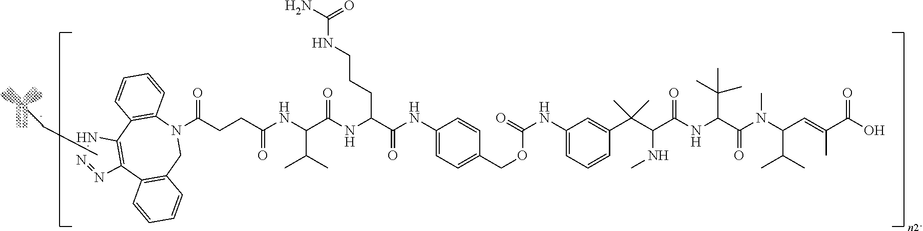

In some embodiments, the antibody of the antibody conjugate comprises the V_{H}/V_{L} pair SEQ ID NOs: 2727/3061.

In some embodiments, an antibody conjugate is provided that comprises: (a) an antibody comprising a V_{H} comprising: a CDR-H1 comprising at least one of SEQ ID NOs: 105 and 430; a CDR-H2 comprising at least one of SEQ ID NOs: 755 and 1080; and a CDR-H3 comprising at least one of SEQ ID NOs: 1405 and 1730; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

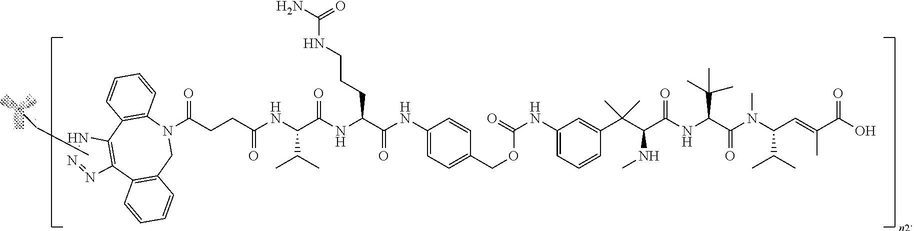

In some embodiments, the antibody of the antibody conjugate comprises the V_{H}/V_{L} pair SEQ ID NOs: 2727/3061.

In some embodiments, an antibody conjugate is provided that comprises: (a) an antibody comprising a V_{H} comprising: a CDR-H1 comprising at least one of SEQ ID NOs: 114 and 439; a CDR-H2 comprising at least one of SEQ ID NOs: 764 and 1089; and a CDR-H3 comprising at least one of SEQ ID NOs: 1414 and 1739; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

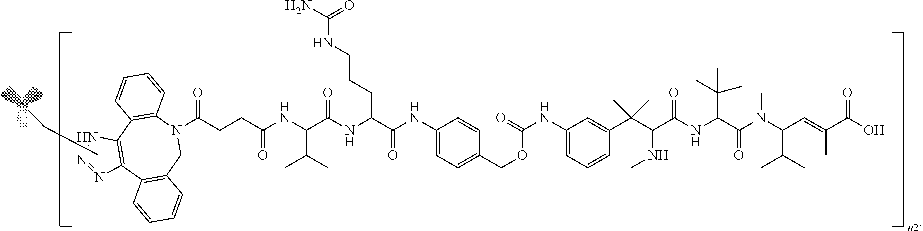

In some embodiments, the antibody of the antibody conjugate comprises the V_{H}/V_{L} pair SEQ ID NOs: 2736/3061.

In some embodiments, an antibody conjugate is provided that comprises: (a) an antibody comprising a V_{H} comprising: a CDR-H1 comprising at least one of SEQ ID NOs: 114 and 439; a CDR-H2 comprising at least one of SEQ ID NOs: 764 and 1089; and a CDR-H3 comprising at least one of SEQ ID NOs: 1414 and 1739; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

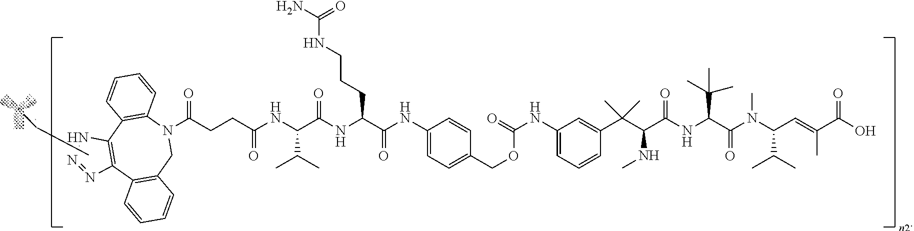

In some embodiments, the antibody of the antibody conjugate comprises the V_{H}/V_{L} pair SEQ ID NOs: 2736/3061.

In some embodiments, an antibody conjugate is provided that comprises: (a) an antibody comprising a V_{H} comprising: a CDR-H1 comprising at least one of SEQ ID NOs: 117 and 442; a CDR-H2 comprising at least one of SEQ ID NOs: 767 and 1092; and a CDR-H3 comprising at least one of SEQ ID NOs: 1417 and 1742; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

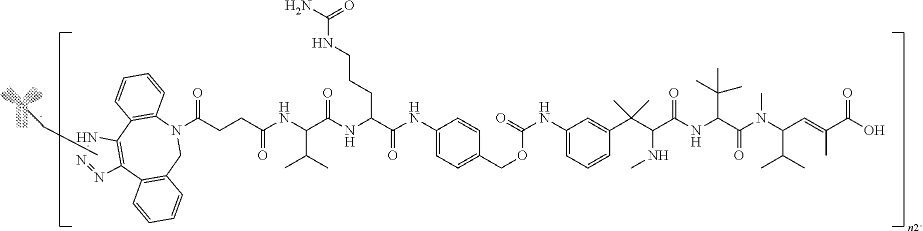

In some embodiments, the antibody of the antibody conjugate comprises the V_{H}/V_{L} pair SEQ ID NOs: 2739/3061.

In some embodiments, an antibody conjugate is provided that comprises: (a) an antibody comprising a V_{H} comprising: a CDR-H1 comprising at least one of SEQ ID NOs: 117 and 442; a CDR-H2 comprising at least one of SEQ ID NOs: 767 and 1092; and a CDR-H3 comprising at least one of SEQ ID NOs: 1417 and 1742; and a V_{L} comprising: a CDR-L1 comprising SEQ ID NO: 2064; a CDR-L2 comprising SEQ ID NO: 2288; and a CDR-L3 comprising SEQ ID NO: 2512; (b) para-azidomethylphenylalanine residues at antibody sites HC180 and HC404; and (c) a linker-payload, wherein n2 is 4 and each linker-payload is bonded to one of the para-azidomethylphenylalanine residue side chains:

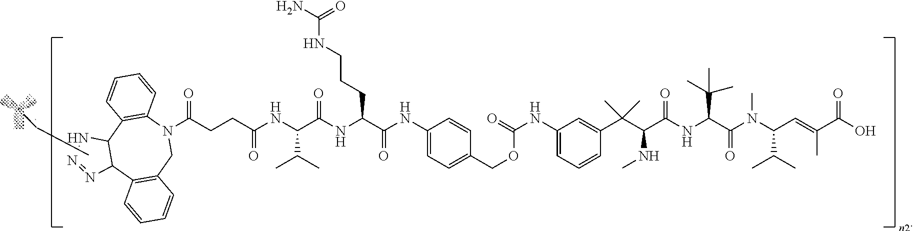

In some embodiments, the antibody of the antibody conjugate comprises the V_{H}/V_{L} pair SEQ ID NOs: 2739/3061.

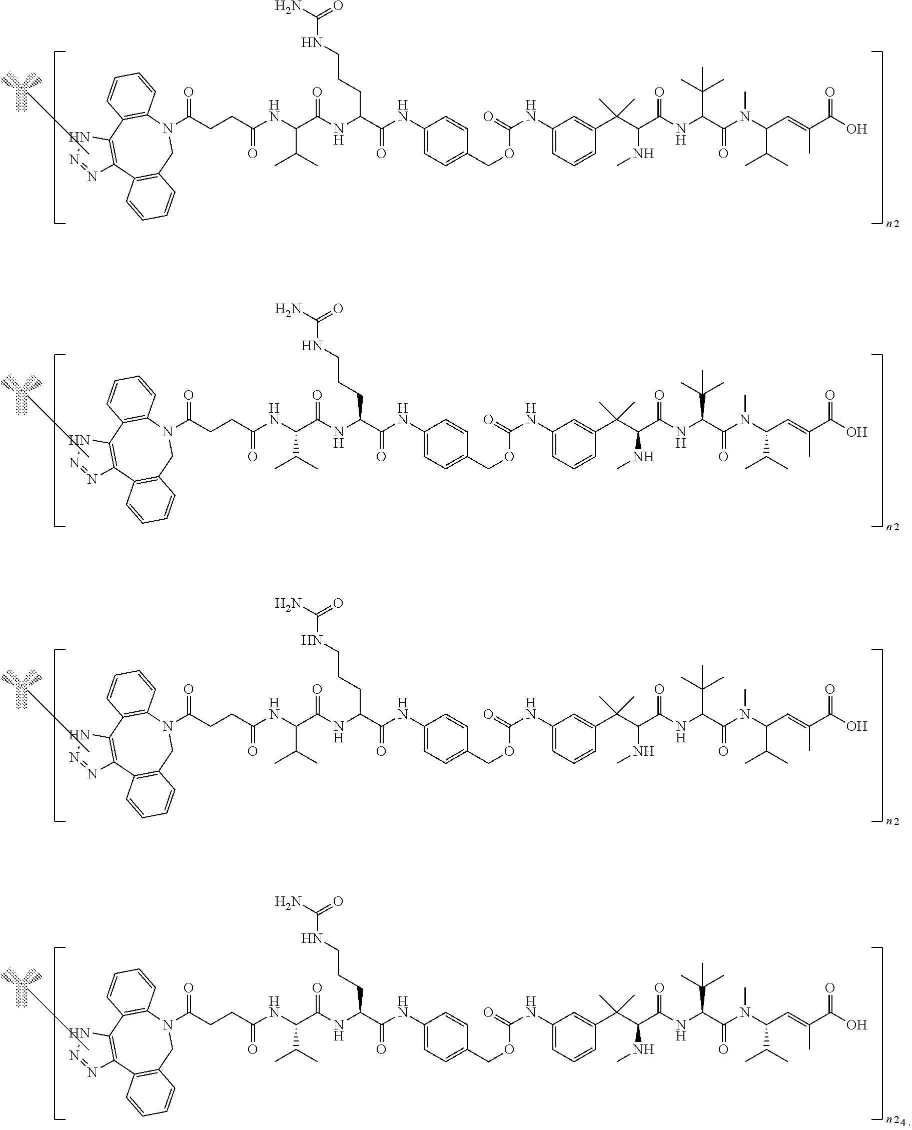

### Payloads

In addition to the payloads described above, the molecular payload can be any molecular entity that one of skill in the art might desire to conjugate to the polypeptide. In certain embodiments, the payload is a therapeutic moiety. In such embodiment, the antibody conjugate can be used to target the therapeutic moiety to its molecular target. In certain embodiments, the payload is a labeling moiety. In such embodiments, the antibody conjugate can be used to detect binding of the polypeptide to its target. In certain embodiments, the payload is a cytotoxic moiety. In such embodiments, the antibody conjugate can be used target the cytotoxic moiety to a diseased cell, for example a cancer cell, to initiate destruction or elimination of the cell. Conjugates comprising other molecular payloads apparent to those of skill in the art are within the scope of the conjugates described herein.

In certain embodiments, an antibody conjugate can have a payload selected from the group consisting of a label, a dye, a polymer, a water-soluble polymer, polyethylene glycol, a derivative of polyethylene glycol, a photocrosslinker, a cytotoxic compound, a radionuclide, a drug, an affinity label, a photoaffinity label, a reactive compound, a resin, a second protein or polypeptide or polypeptide analog, an antibody or antibody fragment, a metal chelator, a cofactor, a fatty acid, a carbohydrate, a polynucleotide, a DNA, a RNA, an antisense polynucleotide, a peptide, a water-soluble dendrimer, a cyclodextrin, an inhibitory ribonucleic acid, a biomaterial, a nanoparticle, a spin label, a fluorophore, a metal-containing moiety, a radioactive moiety, a novel functional group, a group that covalently or noncovalently interacts with other molecules, a photocaged moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, a small molecule, or any combination thereof. In an embodiment, the payload is a label, a dye, a polymer, a cytotoxic compound, a radionuclide, a drug, an affinity label, a resin, a protein, a polypeptide, a polypeptide analog, an antibody, antibody fragment, a metal chelator, a cofactor, a fatty acid, a carbohydrate, a polynucleotide, a DNA, a RNA, a peptide, a fluorophore, or a carbon-linked sugar. In another embodiment, the payload is a label, a dye, a polymer, a drug, an antibody, antibody fragment, a DNA, an RNA, or a peptide.

Useful drug payloads include any cytotoxic or cytostatic agent. Useful classes of cytotoxic agents include, for example, antitubulin agents, auristatins, DNA minor groove binders, DNA replication inhibitors, alkylating agents (e.g., platinum complexes such as cisplatin, mono (platinum), bis (platinum) and tri-nuclear platinum complexes and carboplatin), anthracyclines, antibiotics, antifolates, antimetabolites, calmodulin inhibitors, chemotherapy sensitizers, duocarmycins, etoposides, fluorinated pyrimidines, ionophores, lexitropsins, maytansinoids, nitrosoureas, platinols, pore-forming compounds, purine antimetabolites, puromycins, radiation sensitizers, rapamycins, steroids, taxanes, topoisomerase inhibitors, vinca alkaloids, or the like.

Individual cytotoxic agents include, for example, an androgen, anthramycin (AMC), asparaginase, 5-azacytidine, azathioprine, bleomycin, busulfan, buthionine sulfoximine, calicheamicin, calicheamicin derivatives, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, DM1, DM4, docetaxel, doxorubicin, etoposide, an estrogen, 5-fluordeoxyuridine, 5-fluorouracil, gemcitabine, gramicidin D, hydroxyurea, idarubicin, ifosfamide, irinotecan, lomustine (CCNU), maytansine, mechlorethamine, melphalan, 6-mercaptopurine, methotrexate, mithramycin, mitomycin C, mitoxantrone, nitroimidazole, paclitaxel, palytoxin, plicamycin, procarbizine, rhizoxin, streptozotocin, tenoposide, 6-thioguanine, thioTEPA, topotecan, vinblastine, vincristine, vinorelbine, VP-16 and VM-26.

In some embodiments, suitable cytotoxic agents include, for example, DNA minor groove binders (e.g., enediynes and lexitropsins, a CBI compound; see also U.S. Pat. No. 6,130,237), duocarmycins, taxanes (e.g., paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epothilone A and B, estramustine, cryptophycins, cemadotin, maytansinoids, discodermolide, eleutherobin, and mitoxantrone.

In some embodiments, the payload is an anti-tubulin agent. Examples of anti-tubulin agents include, but are not limited to, taxanes (e.g., Taxol® (paclitaxel), Taxotere® (docetaxel)), T67 (Tularik) and vinca alkyloids (e.g., vincristine, vinblastine, vindesine, and vinorelbine). Other antitubulin agents include, for example, baccatin derivatives, taxane analogs, epothilones (e.g., epothilone A and B), nocodazole, colchicine and colcimid, estramustine, cryptophycins, cemadotin, maytansinoids, combretastatins, discodermolide, and eleutherobin.

In certain embodiments, the cytotoxic agent is a maytansinoid, another group of anti-tubulin agents. For example, in specific embodiments, the maytansinoid can be maytansine or DM1 (ImmunoGen, Inc.; see also Chari et al., 1992, Cancer Res. 52:127-131).

In some embodiments, the payload is an auristatin, such as auristatin E or a derivative thereof. For example, the auristatin E derivative can be an ester formed between auristatin E and a keto acid. For example, auristatin E can be reacted with paraacetyl benzoic acid or benzoylvaleric acid to produce AEB and AEVB, respectively. Other typical auristatin derivatives include AFP (auristatin phenylalanine phenylenediamine), MMAF (monomethyl auristatin F), and MMAE (monomethyl auristatin E). The synthesis and structure of auristatin derivatives are described in U.S. Patent Application Publication Nos. 2003-0083263, 2005-0238649 and 2005-0009751; International Patent Publication No. WO 04/010957, International Patent Publication No. WO 02/088172, and U.S. Pat. Nos. 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414.

In some embodiments, the payload is a hemiasterlin. Hemiasterlins suitable for use in the antibody-drug conjugates described herein are described, for example, in International Patent Publication No. WO 2016/123582, which is incorporated herein by reference in its entirety.

In some embodiments, the payload is not a radioisotope. In some embodiments, the payload is not radioactive.

In some embodiments, the payload is an antimetabolite. The antimetabolite can be, for example, a purine antagonist (e.g., azothioprine or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscarnet, or trifluridine.

In other embodiments, the payload is tacrolimus, cyclosporine, FU506 or rapamycin. In further embodiments, the Drug is aldesleukin, alemtuzumab, alitretinoin, allopurinol, altretamine, amifostine, anastrozole, arsenic trioxide, bexarotene, bexarotene, calusterone, capecitabine, celecoxib, cladribine, Darbepoetin alfa, Denileukin diftitox, dexrazoxane, dromostanolone propionate, epirubicin, Epoetin alfa, estramustine, exemestane, Filgrastim, floxuridine, fludarabine, fulvestrant, gemcitabine, gemtuzumab ozogamicin (MYLOTARG), goserelin, idarubicin, ifosfamide, imatinib mesylate, Interferon alfa-2a, irinotecan, letrozole, leucovorin, levamisole, meclorethamine or nitrogen mustard, megestrol, mesna, methotrexate, methoxsalen, mitomycin C, mitotane, nandrolone phenpropionate, oprelvekin, oxaliplatin, pamidronate, pegademase, pegaspargase, pegfilgrastim, pentostatin, pipobroman, a pladienolide, plicamycin, porfimer sodium, procarbazine, quinacrine, rasburicase, Rituximab, Sargramostim, streptozocin, tamoxifen, temozolomide, teniposide, testolactone, thioguanine, toremifene, Tositumomab, Trastuzumab (HERCEPTIN), tretinoin, uracil mustard, valrubicin, vinblastine, vincristine, vinorelbine or zoledronate.

Other useful drug payloads include chemical compounds useful in the treatment of cancer. Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially uncialamycin, calicheamicin gammall, and calicheamicin omegall (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pladienolide B, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR®(gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Other useful payloads include: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4 (5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); (x) agents that act to regulate or inhibit activity of members of the poly (ADP-ribose) polymerase (PARP) family in tumors (e.g., Talazoparib (BMN-673), Iniparib (BSI 201), Veliparib (ABT-888), Olaparib (AZD-2281, trade name LYNPARZA™), Rucaparib (AG 014699), BGB-290, E7016, E7449, and CEP-9722); (xi) agents that act to regulate or inhibit activity of members of the histone deacetylase (HDAC) family in tumors (e.g., abexinostat, entinostat, gavinostat, 4SC-202, ACY-241, AR-42, CG200745, CHR-2845, CHR-3996, CXD101, MPTOE028, OBP-801, SHP-141, CUDC-101, KA2507, panobinostat, pracinostat, quisinostat, resminostat, ricolinostat); (xii) agents that act to regulate or inhibit activity of mitochondrial enzyme isocitrate dehydrogenase type 2 (IDH2) in tumors (e.g., enasidenib mesylate (CC-90007, AG-221 mesylate); and (xiii) pharmaceutically acceptable salts, acids and derivatives of any of the above. Other useful payloads include anti-angiogenic agents, including, e.g., MMP-2 (matrix-metalloproteinase 2) inhibitors, MMP-9 (matrix-metalloproteinase 9) inhibitors, COX-II (cyclooxygenase II) inhibitors, and VEGF receptor tyrosine kinase inhibitors. Examples of such useful matrix metalloproteinase inhibitors that can be used in combination with the present compounds/compositions are described in WO 96/33172, WO 96/27583, EP 818442, EP 1004578, WO 98/07697, WO 98/03516, WO 98/34918, WO 98/34915, WO 98/33768, WO 98/30566, EP 606,046, EP 931,788, WO 90/05719, WO 99/52910, WO 99/52889, WO 99/29667, WO 99/07675, EP 945864, U.S. Pat. Nos. 5,863,949, 5,861,510, and EP 780,386, all of which are incorporated herein in their entireties by reference. Examples of VEGF receptor tyrosine kinase inhibitors include 4-(4-bromo-2-fluoroanilino)-6-methoxy-7-(1-methylpiperidin-4-ylmethoxy) quinazoline (ZD6474; Example 2 within WO 01/32651), 4-(4-fluoro-2-methylindol-5-yloxy)-6-methoxy-7-(3-pyrrolidin-1-ylpropoxy) quinazoline (AZD2171; Example 240 within WO 00/47212), vatalanib (PTK787; WO 98/35985) and SU11248 (sunitinib; WO 01/60814), and compounds such as those disclosed in PCT Publication Nos. WO 97/22596, WO 97/30035, WO 97/32856, and WO 98/13354).

In certain embodiments, the payload is selected from the group consisting of maytansine, hemiasterlin, amanitin, exatecan, deruxtecan (DXd), anthracycline, PNU-159682, pyrrolobenzodiazepine (PBD), MMAF, and MMAE. In certain embodiments, the payload is maytansine. In certain embodiments, the payload is hemiasterlin. In certain embodiments, the payload is amanitin. In certain embodiments, the payload is hemiasterlin. In certain embodiments, the payload is amanitin. In certain embodiments, the payload is hemiasterlin. In certain embodiments, the payload is deruxtecan. In certain embodiments, the payload is hemiasterlin. In certain embodiments, the payload is anthracycline. In certain embodiments, the payload is hemiasterlin. In certain embodiments, the payload is PNU-159682. In certain embodiments, the payload is hemiasterlin. In certain embodiments, the payload is pyrrolobenzodiazepine. In certain embodiments, the payload is MMAF. In certain embodiments, the payload is MMAE.

In certain embodiments, the payload is an antibody or an antibody fragment. In certain embodiments, the payload antibody or fragment can be encoded by any of the immunoglobulin genes recognized by those of skill in the art. The immunoglobulin genes include, but are not limited to, the κ, λ, α, γ (IgG1, IgG2, IgG3, and IgG4), δ, ε and μ constant region genes, as well as the immunoglobulin variable region genes. The term includes full-length antibody and antibody fragments recognized by those of skill in the art, and variants thereof. Exemplary fragments include but are not limited to Fv, Fc, Fab, and (Fab′)₂, single chain Fv (scFv), diabodies, triabodies, tetrabodies, bifunctional hybrid polypeptides, CDR1, CDR2, CDR3, combinations of CDR's, variable regions, framework regions, constant regions, and the like.

In certain embodiments, the payload is one or more water-soluble polymers. A wide variety of macromolecular polymers and other molecules can be linked to the polypeptides described herein to modulate biological properties of the polypeptide, and/or provide new biological properties to the polypeptide. These macromolecular polymers can be linked to the polypeptide via a naturally encoded amino acid, via a non-naturally encoded amino acid, or any functional substituent of a natural or modified amino acid, or any substituent or functional group added to a natural or modified amino acid. The molecular weight of the polymer may be of a wide range, including but not limited to, between about 100 Da and about 100,000 Da or more.

The polymer selected may be water soluble so that a protein to which it is attached does not precipitate in an aqueous environment, such as a physiological environment. The polymer may be branched or unbranched. Preferably, for therapeutic use of the end-product preparation, the polymer will be pharmaceutically acceptable.

In certain embodiments, the proportion of polyethylene glycol molecules to polypeptide molecules will vary, as will their concentrations in the reaction mixture. In general, the optimum ratio (in terms of efficiency of reaction in that there is minimal excess unreacted protein or polymer) may be determined by the molecular weight of the polyethylene glycol selected and on the number of available reactive groups available. As relates to molecular weight, typically the higher the molecular weight of the polymer, the fewer number of polymer molecules which may be attached to the protein. Similarly, branching of the polymer should be taken into account when optimizing these parameters. Generally, the higher the molecular weight (or the more branches) the higher the polymer: protein ratio.

The water-soluble polymer may be any structural form including but not limited to linear, forked or branched. Typically, the water soluble polymer is a poly (alkylene glycol), such as poly(ethylene glycol) (PEG), but other water soluble polymers can also be employed. By way of example, PEG is used to describe certain embodiments.

PEG is a well-known, water-soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented as linked to a polypeptide by the formula: XO—(CH₂CH₂O)ₙ—CH₂CH₂—Y where n is 2 to 10,000, X is H or a terminal modification, including but not limited to, a C₁₋₄ alkyl, and Y is the attachment point to the polypeptide.

In some cases, a PEG terminates on one end with hydroxy or methoxy, i.e., X is H or CH₃ (“methoxy PEG”). Alternatively, the PEG can terminate with a reactive group, thereby forming a bifunctional polymer. Typical reactive groups can include those reactive groups that are commonly used to react with the functional groups found in the 20 common amino acids (including but not limited to, maleimide groups, activated carbonates (including but not limited to, p-nitrophenyl ester), activated esters (including but not limited to, N-hydroxysuccinimide, p-nitrophenyl ester, and aldehydes) as well as functional groups that are inert to the 20 common amino acids but that react specifically with complementary functional groups present in non-naturally encoded amino acids (including but not limited to, azide groups, alkyne groups). It is noted that the other end of the PEG, which is shown in the above formula by Y, will attach either directly or indirectly to a polypeptide via a naturally-occurring or non-naturally encoded amino acid. For instance, Y may be an amide, carbamate, or urea linkage to an amine group (including but not limited to, the epsilon amine of lysine or the N-terminus) of the polypeptide. Alternatively, Y may be a maleimide linkage to a thiol group (including but not limited to, the thiol group of cysteine). Alternatively, Y may be a linkage to a residue not commonly accessible via the 20 common amino acids. For example, an azide group on the PEG can be reacted with an alkyne group on the polypeptide to form a Huisgen [3+2] cycloaddition product. Alternatively, an alkyne group on the PEG can be reacted with an azide group present in a non-naturally encoded amino acid, such as the modified amino acids described herein, to form a similar product. In some embodiments, a strong nucleophile (including but not limited to, hydrazine, hydrazide, hydroxylamine, semicarbazide) can be reacted with an aldehyde or ketone group present in a non-naturally encoded amino acid to form a hydrazone, oxime or semicarbazone, as applicable, which in some cases can be further reduced by treatment with an appropriate reducing agent. Alternatively, the strong nucleophile can be incorporated into the polypeptide via a non-naturally encoded amino acid and used to react preferentially with a ketone or aldehyde group present in the water-soluble polymer.

Any molecular mass for a PEG can be used as practically desired, including but not limited to, from about 100 Daltons (Da) to 100,000 Da or more as desired (including but not limited to, sometimes 0.1-50 kDa or 10-40 kDa). Branched chain PEGs, including but not limited to, PEG molecules with each chain having a MW ranging from 1-100 kDa (including but not limited to, 1-50 kDa or 5-20 kDa) can also be used. A wide range of PEG molecules are described in, including but not limited to, the Shearwater Polymers, Inc. catalog, and the Nektar Therapeutics catalog, incorporated herein by reference.

Generally, at least one terminus of the PEG molecule is available for reaction with the antibody. For example, PEG derivatives bearing alkyne and azide moieties for reaction with amino acid side chains can be used to attach PEG to non-naturally encoded amino acids as described herein. If the non-naturally encoded amino acid comprises an azide, then the PEG will typically contain either an alkyne moiety to effect formation of the [3+2] cycloaddition product or an activated PEG species (i.e., ester, carbonate) containing a phosphine group to effect formation of the amide linkage. Alternatively, if the non-naturally encoded amino acid comprises an alkyne, then the PEG will typically contain an azide moiety to effect formation of the [3+2] Huisgen cycloaddition product. If the non-naturally encoded amino acid comprises a carbonyl group, the PEG will typically comprise a potent nucleophile (including but not limited to, a hydrazide, hydrazine, hydroxylamine, or semicarbazide functionality) in order to effect formation of corresponding hydrazone, oxime, and semicarbazone linkages, respectively. In other alternatives, a reverse of the orientation of the reactive groups described herein can be used, i.e., an azide moiety in the non-naturally encoded amino acid can be reacted with a PEG derivative containing an alkyne.

In some embodiments, the polypeptide variant with a PEG derivative contains a chemical functionality that is reactive with the chemical functionality present on the side chain of the non-naturally encoded amino acid.

In certain embodiments, the payload is an azide- or acetylene-containing polymer comprising a water-soluble polymer backbone having an average molecular weight from about 800 Da to about 100,000 Da. The polymer backbone of the water-soluble polymer can be poly (ethylene glycol). However, it should be understood that a wide variety of water soluble polymers including but not limited to poly (ethylene) glycol and other related polymers, including poly (dextran) and poly (propylene glycol), are also suitable for use and that the use of the term PEG or poly (ethylene glycol) is intended to encompass and include all such molecules. The term PEG includes, but is not limited to, poly (ethylene glycol) in any of its forms, including bifunctional PEG, multiarmed PEG, derivatized PEG, forked PEG, branched PEG, pendent PEG (i.e. PEG or related polymers having one or more functional groups pendent to the polymer backbone), or PEG with degradable linkages therein.

The polymer backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, glycerol oligomers, pentaerythritol and sorbitol. The central branch moiety can also be derived from several amino acids, such as lysine. The branched poly (ethylene glycol) can be represented in general form as R(-PEG-OH)ₘ in which R is derived from a core moiety, such as glycerol, glycerol oligomers, or pentaerythritol, and m represents the number of arms. Multi-armed PEG molecules, such as those described in U.S. Pat. Nos. 5,932,462 5,643,575; 5,229,490; 4,289,872; U.S. Pat. Appl. 2003/0143596; WO 96/21469; and WO 93/21259, each of which is incorporated by reference herein in its entirety, can also be used as the polymer backbone.

Branched PEG can also be in the form of a forked PEG represented by PEG(-YCHZ₂)ₙ, where Y is a linking group and Z is an activated terminal group linked to CH by a chain of atoms of defined length.

Yet another branched form, the pendant PEG, has reactive groups, such as carboxyl, along the PEG backbone rather than at the end of PEG chains.

In addition to these forms of PEG, the polymer can also be prepared with weak or degradable linkages in the backbone. For example, PEG can be prepared with ester linkages in the polymer backbone that are subject to hydrolysis. As shown herein, this hydrolysis results in cleavage of the polymer into fragments of lower molecular weight: -PEG-CO₂-PEG-+H₂O→PEG-CO₂H+HO-PEG- It is understood by those skilled in the art that the term poly (ethylene glycol) or PEG represents or includes all the forms known in the art including but not limited to those disclosed herein.

Many other polymers are also suitable for use. In some embodiments, polymer backbones that are water-soluble, with from 2 to about 300 termini, are particularly suitable. Examples of suitable polymers include, but are not limited to, other poly (alkylene glycols), such as poly (propylene glycol) (“PPG”), copolymers thereof (including but not limited to copolymers of ethylene glycol and propylene glycol), terpolymers thereof, mixtures thereof, and the like. Although the molecular weight of each chain of the polymer backbone can vary, it is typically in the range of from about 800 Da to about 100,000 Da, often from about 6,000 Da to about 80,000 Da.

Those of ordinary skill in the art will recognize that the foregoing list for substantially water-soluble backbones is by no means exhaustive and is merely illustrative, and that all polymeric materials having the qualities described herein are contemplated as being suitable for use.

In some embodiments the polymer derivatives are “multi-functional”, meaning that the polymer backbone has at least two termini, and possibly as many as about 300 termini, functionalized or activated with a functional group. Multifunctional polymer derivatives include, but are not limited to, linear polymers having two termini, each terminus being bonded to a functional group which may be the same or different.

In certain embodiments, the antibody conjugate can comprise one, two, three, four, five, six, seven, eight, nine, ten or more payloads per antibody. In certain embodiments, the antibody conjugate can comprise one payload per antibody. In certain embodiments, the antibody conjugate can comprise two payloads per antibody. In certain embodiments, the antibody conjugate can comprise four payloads per antibody. In certain embodiments, the antibody conjugate can comprise six payloads per antibody. In certain embodiments, the antibody conjugate can comprise eight payloads per antibody.

### 5. Linkers

In certain embodiments, the antibodies can be linked to the payloads with one or more linkers capable of reacting with an antibody amino acid and with a payload group. The one or more linkers can be any linkers apparent to those of skill in the art.

The term “linker” is used herein to refer to groups or bonds that normally are formed as the result of a chemical reaction and typically are covalent linkages.

Useful linkers include those described herein. In certain embodiments, the linker is any divalent or multivalent linker known to those of skill in the art. Useful divalent linkers include alkylene, substituted alkylene, heteroalkylene, substituted heteroalkylene, arylene, substituted arylene, heteroarlyene, and substituted heteroarylene. In certain embodiments, the linker is C₁₋₁₀ alkylene or C₁₋₁₀ heteroalkylene. In some embodiments, the C₁₋₁₀heteoalkylene is PEG.

In certain embodiments, the linker is hydrolytically stable. Hydrolytically stable linkages means that the linkages are substantially stable in water and do not react with water at useful pH values, including but not limited to, under physiological conditions for an extended period of time, perhaps even indefinitely. In certain embodiments, the linker is hydrolytically unstable. Hydrolytically unstable or degradable linkages mean that the linkages are degradable in water or in aqueous solutions, including for example, blood. Enzymatically unstable or degradable linkages mean that the linkage can be degraded by one or more enzymes.

As understood in the art, PEG and related polymers may include degradable linkages in the polymer backbone or in the linker group between the polymer backbone and one or more of the terminal functional groups of the polymer molecule. For example, ester linkages formed by the reaction of PEG carboxylic acids or activated PEG carboxylic acids with alcohol groups on a biologically active agent generally hydrolyze under physiological conditions to release the agent.

Other hydrolytically degradable linkages include, but are not limited to, carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

A number of different cleavable linkers are known to those of skill in the art. See U.S. Pat. Nos. 4,618,492; 4,542,225, and 4,625,014. The mechanisms for release of an agent from these linker groups include, for example, irradiation of a photolabile bond and acid-catalyzed hydrolysis. U.S. Pat. No. 4,671,958, for example, includes a description of immunoconjugates comprising linkers which are cleaved at the target site in vivo by the proteolytic enzymes of the patient's complement system. The length of the linker may be predetermined or selected depending upon a desired spatial relationship between the polypeptide and the molecule linked to it. In view of the large number of methods that have been reported for attaching a variety of radiodiagnostic compounds, radiotherapeutic compounds, drugs, toxins, and other agents to polypeptides one skilled in the art will be able to determine a suitable method for attaching a given agent to a polypeptide.

The linker may have a wide range of molecular weight or molecular length. Larger or smaller molecular weight linkers may be used to provide a desired spatial relationship or conformation between the polypeptide and the linked entity. Linkers having longer or shorter molecular length may also be used to provide a desired space or flexibility between the polypeptide and the linked entity. Similarly, a linker having a particular shape or conformation may be utilized to impart a particular shape or conformation to the polypeptide or the linked entity, either before or after the polypeptide reaches its target. The functional groups present on each end of the linker may be selected to modulate the release of a polypeptide or a payload under desired conditions. This optimization of the spatial relationship between the polypeptide and the linked entity may provide new, modulated, or desired properties to the molecule.

In some embodiments, provided herein are water-soluble bifunctional linkers that have a dumbbell structure that includes: a) an azide, an alkyne, a hydrazine, a hydrazide, a hydroxylamine, or a carbonyl-containing moiety on at least a first end of a polymer backbone; and b) at least a second functional group on a second end of the polymer backbone. The second functional group can be the same or different as the first functional group. The second functional group, in some embodiments, is not reactive with the first functional group. In some embodiments, water-soluble compounds that comprise at least one arm of a branched molecular structure are provided. For example, the branched molecular structure can be a dendritic structure.

In some embodiments, the linker is derived from a linker precursor selected from the group consisting of: N-succinimidyl-3-(2-pyridyldithio) propionate (SPDP), N-succinimidyl 4-(2-pyridyldithio) pentanoate (SPP), N-succinimidyl 4-(2-pyridyldithio) butanoate (SPDB), N-succinimidyl-4-(2-pyridyldithio)-2-sulfo-butanoate (sulfo-SPDB), N-succinimidyl iodoacetate (SIA), N-succinimidyl (4-iodoacetyl) aminobenzoate (SIAB), maleimide PEG NHS, N-succinimidyl 4-(maleimidomethyl) cyclohexanecarboxylate (SMCC), N-sulfosuccinimidyl 4-(maleimidomethyl) cyclohexanecarboxylate (sulfo-SMCC) or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1). In a specific embodiment, the linker is derived from the linker precursor N-succinimidyl 4-(maleimidomethyl) cyclohexanecarboxylate (SMCC).

In some embodiments, the linker is derived from a linker precursor selected from the group consisting of dipeptides, tripeptides, tetrapeptides, and pentapeptides. In such embodiments, the linker can be cleaved by a protease. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit), glycine-glycine-glycine (gly-gly-gly), and glycine-methoxyethoxyethyl) serine-valine (gly-val-citalanine OMESerValAla).

In some embodiments, a linker comprises a self-immolative spacer. In certain embodiments, the self-immolative spacer comprises p-aminobenzyl. In some embodiments, a p-aminobenzyl alcohol is attached to an amino acid unit via an amide bond, and a carbamate, methylcarbamate, or carbonate is made between the benzyl alcohol and the payload (Hamann et al. (2005) Expert Opin. Ther. Patents (2005) 15:1087-1103). In some embodiments, the linker comprises p-aminobenzyloxycarbonyl (PAB). Other examples of self-immolative spacers include, but are not limited to, aromatic compounds that are electronically similar to the PAB group, such as 2-aminoimidazol-5-methanol derivatives (U.S. Pat. No. 7,375,078; Hay et al. (1999) Bioorg. Med. Chem. Lett. 9:2237) and ortho- or para-aminobenzylacetals. In some embodiments, spacers can be used that undergo cyclization upon amide bond hydrolysis, such as substituted and unsubstituted 4-aminobutyric acid amides (Rodrigues et al. (1995) Chemistry Biology 2:223), appropriately substituted bicyclo[2.2.1] and bicyclo[2.2.2]ring systems (Storm et al. (1972) J. Amer. Chem. Soc. 94:5815) and 2-aminophenylpropionic acid amides (Amsberry, et al. (1990) J. Org. Chem. 55:5867). Linkage of a drug to the α-carbon of a glycine residue is another example of a self-immolative spacer that may be useful in conjugates (Kingsbury et al. (1984) J. Med. Chem. 27:1447).

In certain embodiments, linker precursors can be combined to form larger linkers. For instance, in certain embodiments, linkers comprise the dipeptide valine-citrulline and p-aminobenzyloxycarbonyl. These are also referenced as citValCit--PAB linkers.

In certain embodiments, the payloads can be linked to the linkers, referred to herein as a linker-payload, with one or more linker groups capable of reacting with an antibody amino acid group. The one or more linkers can be any linkers apparent to those of skill in the art or those set forth herein.

Additional linkers are disclosed herein, such as, for example, the linker precursors (A)-(L) described below.

### 6. Germline

In some embodiments, the antibody or antibody of the conjugate that specifically binds Tissue Factor is an antibody comprising a variable region that is encoded by a particular germline gene, or a variant thereof. The illustrative antibodies provided herein comprise variable regions that are encoded by the heavy chain variable region germline genes V_{H}1-18, V_{H}3-33, V_{H}2-5, V_{H}2-70, and V_{H}4-30-4. or variants thereof; and the light chain variable region germline genes Vκ1-5, Vκ3-11, Vκ2-20, Vκ1-33, and Vκ1-16, or variants thereof.

One of skill in the art would recognize that the CDR sequences provided herein may also be useful when combined with variable regions encoded by other variable region germline genes, or variants thereof. In particular, the CDR sequences provided herein may be useful when combined with variable regions encoded by variable region germline genes, or variants thereof, that are structurally similar to the variable region germline genes recited above. For example, in some embodiments, a CDR-H sequence provided herein may be combined with a variable region encoded by a variable region germline gene selected from the V_{H} 1, V_{H} 2, V_{H} 3, or V_{H} 4 families, or a variant thereof. In some embodiments, a CDR-L sequence provided herein may be combined with a variable region encoded by a variable region germline gene selected from the Vκ1, Vκ2, or Vκ3, or a variant thereof.

### 7. Affinity

In some embodiments, the affinity of the antibody for tissue factor as indicated by K_{D}, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. In some embodiments, the affinity of the antibody is between about 10⁻⁷ M and 10⁻¹¹ M. In some embodiments, the affinity of the antibody is between about 10⁻⁷ M and 10⁻¹⁰ M. In some embodiments, the affinity of the antibody is between about 10⁻⁷ M and 10⁻⁹ M. In some embodiments, the affinity of the antibody is between about 10⁻⁷ M and 10⁻⁸ M. In some embodiments, the affinity of the antibody is between about 10⁻⁸ M and 10⁻¹¹ M. In some embodiments, the affinity of the antibody is between about 10⁻⁸ M and 10⁻¹⁰ M. In some embodiments, the affinity of the antibody is between about 10⁻⁹ M and 10⁻¹¹ M. In some embodiments, the affinity of the antibody is between about 10⁻¹⁰ M and 10⁻¹¹ M.

In some embodiments, the affinity of the antibody for human tissue factor, as determined by surface plasmon resonance at 25° C., and as indicated by K_{D}, is from about 3.59×10⁻⁹ M to about 1.91×10⁻⁸ M. In some embodiments, the affinity of the antibody for human tissue factor is about 3.59×10⁻⁹ M, about 3.71×10⁻⁹ M, about 3.73×10⁻⁹ M, about 4.52×10⁻⁹ M, about 4.77×10⁻⁹ M, about 5.42×10⁻⁹ M, about 6.24×10⁻⁹ M, about 7.30×10⁻⁹ M, about 7.78×10⁻⁹ M, about 7.81×10⁻⁹ M, about 1.38×10⁻⁸ M, about 1.74×10⁻⁸ M, or about 1.91×10⁻⁸ M.

In some embodiments, the affinity of the antibody for cynomolgus tissue factor, as determined by surface plasmon resonance at 25° C., and as indicated by K_{D}, is from about 2.55×10⁻¹⁰ M to about 6.07×10⁻⁸ M. In some embodiments, the affinity of the antibody for human tissue factor is about 2.55×10⁻¹⁰ M, about 1.12×10⁻⁹ M, about 1.19×10⁻⁹ M, about 1.84×10⁻⁹ M, about 2.61×10⁻⁹ M, about 7.33×10⁻⁹ M, about 1.56×10⁻⁸ M, about 1.89×10⁻⁸ M, about 2.01×10⁻⁸ M, about 2.42×10⁻⁸ M, about 2.99×10⁻⁸ M, about 4.16×10⁻⁸ M, or about 6.07×10⁻⁸ M.

In some embodiments the antibody has a kₐ of at least about 10⁴ M⁻¹×sec⁻¹. In some embodiments the antibody has a kₐ of at least about 10⁵ M⁻¹×sec⁻¹. In some embodiments the antibody has a kₐ of at least about 10⁶ M⁻¹×sec⁻¹. In some embodiments the antibody has a kₐ of between about 10⁴ M⁻¹×sec⁻¹ and about 105 M⁻¹×sec⁻¹. In some embodiments the antibody has a kₐ of between about 105 M⁻¹×sec⁻¹ and about 106 M⁻¹×sec⁻¹.

In some embodiments the antibody has a kₐ when associating with human tissue factor, as determined by surface plasmon resonance at 25° C., of from about 5.4×10⁴ M⁻¹×sec⁻¹ to about 5.47×10⁵ M⁻¹×sec⁻¹. In some embodiments the antibody has a kₐ when associating with human tissue factor of about 5.4×10⁴ M⁻¹×sec⁻¹, about 5.72×10⁴ M⁻¹×sec⁻¹, about 6.25×10⁴ M⁻¹×sec⁻¹, about 7.12×10⁴ M⁻¹×sec⁻¹, about 1.88×10⁵ M⁻¹×sec⁻¹, about 3.03×10⁵ M⁻¹×sec⁻¹, about 3.11×10⁵ M⁻¹×sec⁻¹, about 3.62×10⁵ M⁻¹×sec⁻¹, about 4.03×10⁵ M⁻¹×sec⁻¹, about 4.87×10⁵ M⁻¹×sec⁻¹, about 5.4×10⁵ M⁻¹×sec⁻¹, or about 5.47×10⁵ M⁻¹×sec⁻¹.

In some embodiments the antibody has a kₐ when associating with cynomolgus tissue factor, as determined by surface plasmon resonance at 25° C., of from about 7.24×10⁴ M⁻¹×sec⁻¹ to about 1.21×10⁶ M⁻¹×sec⁻¹. In some embodiments the antibody has a kₐ when associating with cynomolgus tissue factor of about 7.24×10⁴ M⁻¹×sec⁻¹, about 8.08×10⁴ M⁻¹×sec⁻¹, about 8.27×10⁴ M⁻¹×sec⁻¹, about 1.28×10⁵ M⁻¹×sec⁻¹, about 1.64×10⁵ M⁻¹×sec⁻¹, about 2.85×10⁵ M⁻¹×sec⁻¹, about 3.16×10⁵ M⁻¹×sec⁻¹, about 4.57×10⁵ M⁻¹×sec⁻¹, about 7.47×10⁵ M⁻¹×sec⁻¹, about 7.52×10⁵ M⁻¹×sec⁻¹, or about 1.21×10⁶ M⁻¹×sec⁻¹.

In some embodiments the antibody has a k_{d} of about 10⁻⁵ sec⁻¹ or less. In some embodiments the antibody has a k_{d} of about 10⁻⁴ sec⁻¹ or less. In some embodiments the antibody has a k_{d} of about 10⁻³ sec⁻¹ or less. In some embodiments the antibody has a k_{d} of between about 10⁻² sec⁻¹ and about 10⁻⁵ sec⁻¹. In some embodiments the antibody has a k_{d} of between about 10⁻² sec⁻¹ and about 104 sec⁻¹. In some embodiments the antibody has a k_{d} of between about 10⁻³ sec⁻¹ and about 10⁻⁵ sec⁻¹.

In some embodiments the antibody has a k_{d} when dissociating from human tissue factor, as determined by surface plasmon resonance at 25° C., of from about 2.75×10⁻⁴ sec⁻¹ to about 2.01×10⁻² sec⁻¹. In some embodiments the antibody has a k_{d} when dissociating from human tissue factor of about 2.75×10⁻⁴ sec⁻¹, about 2.99×10⁻⁴ sec⁻¹, about 3.03×10⁻⁴ sec⁻¹, about 3.61×10⁻⁴ sec⁻¹, about 4.16×10⁻⁴ sec⁻¹, about 4.18×10⁻⁴ sec⁻¹, about 4.22×10⁻⁴ sec⁻¹, about 4.39×10⁻⁴ sec⁻¹, about 6.99×10⁻⁴ sec⁻¹, about 5.11×10⁻¹ sec⁻¹, about 5.23×10⁻⁴ sec⁻¹, about 5.28×10⁻⁴ sec⁻¹, about 5.40×10⁻⁴ sec⁻¹, about 6.04×10⁻⁴ sec⁻¹, about 6.74×10⁻⁴ sec⁻¹, about 6.78×10⁻⁴ sec⁻¹, about 6.91×10⁻⁴ sec⁻¹, about 6.95×10⁻⁴ sec⁻¹, about 7.26×10⁻⁴ sec⁻¹, about 7.27×10⁻⁴ sec⁻¹, about 7.36×10⁻⁴ sec⁻¹, about 7.85×10⁻⁴ sec⁻¹, about 8.42×10⁻⁴ sec⁻¹, about 8.65×10⁻⁴ sec⁻¹, about 9.13×10⁻⁴ sec⁻¹, about 9.78×10⁻⁴ sec⁻¹, about 1.15×10⁻³ sec⁻¹, about 1.16×10⁻³ sec⁻¹, about 1.24×10⁻³ sec⁻¹, about 1.29×10⁻³ sec⁻¹, about 1.31×10⁻³ sec⁻¹, about 1.39×10⁻³ sec⁻¹, about 1.41×10⁻³ sec⁻¹, about 1.44×10⁻³ sec⁻¹, about 1.69×10⁻³ sec⁻¹, about 1.82×10⁻³ sec⁻¹, about 1.86×10⁻³ sec⁻¹, about 1.96×10⁻³ sec⁻¹, about 2.04×10⁻³ sec⁻¹, about 2.05×10⁻³ sec⁻¹, about 2.26×10⁻³ sec⁻¹, about 2.27×10⁻³ sec⁻¹, about 2.50×10⁻³ sec⁻¹, about 2.74×10⁻³ sec⁻¹, about 3.04×10⁻³ sec⁻¹, about 3.13×10⁻³ sec⁻¹, about 3.47×10⁻³ sec⁻¹, about 4.15×10⁻³ sec⁻¹, about 4.23×10⁻³ sec⁻¹, about 4.26×10⁻³ sec⁻¹, about 5.94×10⁻³ sec⁻¹, about 7.74×10⁻³ sec⁻¹, about 8.27×10⁻³ sec⁻¹, about 1.22×10⁻² sec⁻¹, or about 2.01×10⁻² sec⁻¹.

In some embodiments, the antibody has a k_{d} when dissociating from cynomolgus tissue factor, as determined by surface plasmon resonance at 25° C., of from about 3.09×10⁻⁴ sec⁻¹ to 3.93×10⁻² sec⁻¹. In some embodiments, the antibody has a k_{d} when dissociating from cynomolgus tissue factor of about 3.09×10⁻⁴ sec⁻¹, about 4.32×10⁻⁴ sec⁻¹, about 4.59×10⁻⁴ sec⁻¹, about 5.14×10⁻⁴ sec⁻¹, about 5.87×10⁻⁴ sec⁻¹, about 6.05×10⁻⁴ sec⁻¹, about 6.31×10⁻⁴ sec⁻¹, about 6.46×10⁻⁴ sec⁻¹, about 6.48×10⁻⁴ sec⁻¹, about 6.65×10⁻⁴ sec⁻¹, about 6.67×10⁻⁴ sec⁻¹, about 6.89×10⁻⁴ sec⁻¹, about 6.96×10⁻⁴ sec⁻¹, about 7.43×10⁻⁴ sec⁻¹, about 7.61×10⁻⁴ sec⁻¹, about 7.82×10⁻⁴ sec⁻¹, about 8.40×10⁻⁴ sec⁻¹, about 8.43×10⁻⁴ sec⁻¹, about 8.61×10⁻⁴ sec⁻¹, about 8.72×10⁻⁴ sec⁻¹, about 9.46×10⁻⁴ sec⁻¹, about 9.91×10⁻⁴ sec⁻¹, about 9.92×10⁻⁴ sec⁻¹, about 1.07×10⁻³ sec⁻¹, about 1.13×10⁻³ sec⁻¹, about 1.15×10⁻³ sec⁻¹, about 1.17×10⁻³ sec⁻¹, about 1.18×10⁻³ sec⁻¹, about 1.22×10⁻³ sec⁻¹, about 1.25×10⁻³ sec⁻¹, about 1.27×10⁻³ sec⁻¹, about 1.30×10⁻³ sec⁻¹, about 1.41×10⁻³ sec⁻¹, about 1.42×10⁻³ sec⁻¹, about 1.66×10⁻³ sec⁻¹, about 1.69×10⁻³ sec⁻¹, about 1.70×10−3 sec⁻¹, about 1.82×10⁻³ sec⁻¹, about 1.84×10⁻³ sec⁻¹, about 1.87×10⁻³ sec⁻¹, about 1.88×10⁻³ sec⁻¹, about 1.95×10⁻³ sec⁻¹, about 2.00×10⁻³ sec⁻¹, about 2.15×10⁻³ sec⁻¹, about 2.31×10⁻³ sec⁻¹, about 2.41×10⁻³ sec⁻¹, about 2.57×10⁻³ sec⁻¹, about 3.03×10⁻³ sec⁻¹, about 3.09×10⁻³ sec⁻¹, about 3.14×10⁻³ sec⁻¹, about 3.52×10⁻³ sec⁻¹, about 4.20×10⁻³ sec⁻¹, about 5.84×10⁻³ sec⁻¹, about 5.96×10⁻³ sec⁻¹, about 6.81×10⁻³ sec⁻¹, about 7.29×10⁻³ sec⁻¹, about 7.35×10⁻³ sec⁻¹, about 7.82×10⁻³ sec⁻¹, about 1.28×10⁻² sec⁻¹, about 1.30×10⁻² sec⁻¹, about 2.88×10⁻² sec⁻¹, about 3.37×10⁻² sec⁻¹, 6.98×10⁰ sec⁻¹, about 1.43×10² sec⁻¹, about 1.62×10² sec⁻¹, about 2.41×10² sec⁻¹, about 2.63×10² sec⁻¹, about 3.55×10² sec⁻¹, or about 3.93×10² sec⁻¹.

In some embodiments, the antibody conjugate has a kₐ when associating with human tissue factor of about 5.08×10⁵ M⁻¹×sec⁻¹ at a temperature of 25° C.

In some embodiments, the antibody conjugate has a kₐ when associating with cynomolgus tissue factor of about 5.82×10⁵ M⁻¹×sec⁻¹ to about 7.47×10⁵ M⁻¹×sec⁻¹ at a temperature of 25° C. In some embodiments, the antibody conjugate has a kₐ when associating with cynomolgus tissue factor of about 5.82×10⁵ M⁻¹×sec⁻¹ or about 7.47×10⁵ M⁻¹×sec⁻¹ at a temperature of 25° C.

In some embodiments, the antibody conjugate has a k_{d} when dissociating from human tissue factor of about 1.89×10⁻³ sec⁻¹ to about 2.09×10⁻³ sec⁻¹ when dissociating from human TF at a temperature of 25° C. In some embodiments, the antibody conjugate has a k_{d} when dissociating from human tissue factor of about 1.89×10⁻³ sec⁻¹ or about 2.09×10⁻³ sec⁻¹ when dissociating from human TF at a temperature of 25° C.

In some embodiments, the antibody conjugate has a k_{d} when dissociating from cynomolgus tissue factor of about 8.92×10⁻⁴ sec⁻¹ to about 1.03×10⁻³ sec⁻¹ when dissociating from cynomolgus TF at a temperature of 25° C. In some embodiments, the antibody conjugate has a k_{d} when dissociating from cynomolgus tissue factor of about 8.92×10⁻⁴ sec⁻¹ or about 1.03×10⁻³ sec⁻¹ when dissociating from cynomolgus TF at a temperature of 25° C.

In some embodiments, the antibody conjugate has an affinity to human tissue factor (K_{D}) of about 3.71×10⁻⁹ M to about 4.11×10⁻⁹ M when bound to human TF at a temperature of 25° C. In some embodiments, the antibody conjugate has a affinity to human tissue factor (K_{D}) of about 3.71×10⁻⁹ M or about 4.11×10⁻⁹ M when bound to human TF at a temperature of 25° C.

In some embodiments, the antibody conjugate has an affinity to cynomolgus tissue factor (K_{D}) of about 1.12×10⁻⁹ M to about 1.77×10⁻⁹ M when bound to human TF at a temperature of 25° C. In some embodiments, the antibody conjugate has an affinity to cynomolgus tissue factor (K_{D}) of about 1.12×10⁻⁹ M, about 1.19×10⁻⁹ M, or about 1.77×10⁻⁹ M when bound to cynomolgus TF at a temperature of 25° C.

In some aspects, the K_{D}, kₐ, and k_{d} are determined at 25° C. In some embodiments, the K_{D}, kₐ, and k_{d} are determined by surface plasmon resonance. In some embodiments, the K_{D}, kₐ, and k_{d} are determined according to the methods described in the Examples provided herein.

### 8. Epitope Bins

In some embodiments, the antibody binds the same epitope as the scFv antibody provided in SEQ ID NOs: 3073 and 3079. In some embodiments, the antibody binds to a different epitope from the scFv antibody provided in SEQ ID NOs: 3073 and 3079. In some embodiments, the antibody binds the same epitope as an antibody encompassing any of SEQ ID NOs: 2727, 2736, 2739, 2751-2838, 2840, 2846, 2851, 2857, and 2859-2860. In some embodiments, the antibody binds the same epitope as an antibody comprising any of the V_{H}-V_{L} pairs, above. In some embodiments, the antibody binds to part of the epitope bound by the scFv antibody provided in SEQ ID NOs: 3073 and 3079. In some embodiments, the antibody competes for epitope binding with the scFv antibody provided in SEQ ID NOs: 3073 and 3079. In some embodiments, the antibody does not compete for epitope binding with scFv antibody provided in SEQ ID NOs: 3073 and 3079. In some embodiments, the antibody competes for epitope binding with an antibody encompassing any of SEQ ID NOs: 2727, 2736, 2739, 2751-2838, 2840, 2846, 2851, 2857, and 2859-2860. In some embodiments, the antibody competes for epitope binding with an antibody comprising any of the V_{H}-V_{L} pairs, above.

### 9. Glycosylation Variants

In certain embodiments, an antibody may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

### 10. Fc Variants

In certain embodiments, amino acid modifications may be introduced into the Fc region of an antibody provided herein to generate an Fc region variant. In certain embodiments, the Fc region variant possesses some, but not all, effector functions. Such antibodies may be useful, for example, in applications in which the half-life of the antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

In some embodiments, the Fc comprises one or more modifications in at least one of the CH3 sequences. In some embodiments, the Fc comprises one or more modifications in at least one of the CH2 sequences. For example, the Fc can include one or modifications selected from the group consisting of: V262E, V262D, V262K, V262R, V262S, V264S, V303R, and V305R. In some embodiments, an Fc is a single polypeptide. In some embodiments, an Fc is multiple peptides, e.g., two polypeptides. Exemplary modifications in the Fc region are described, for example, in International Patent Application No. PCT/US2017/037545, filed Jun. 14, 2017.

An alteration in in CDC and/or ADCC activity can be confirmed using in vitro and/or in vivo assays. For example, Fc receptor (FcR) binding assays can be conducted to measure FcγR binding. The primary cells for mediating ADCC, NK cells, express FcγRIII only, whereas monocytes express FcγRI, FcγRII and FcγRIII. FcR expression on hematopoietic cells is summarized in Ravetch and Kinet, Ann. Rev. Immunol., 1991, 9:457-492, incorporated by reference in its entirety.

Non-limiting examples of in vitro assays to assess ADCC activity of a molecule of interest are provided in U.S. Pat. Nos. 5,500,362 and 5,821,337; Hellstrom et al., Proc. Natl. Acad. Sci. U.S.A., 1986, 83:7059-7063; Hellstrom et al., Proc. Natl. Acad. Sci. U.S.A., 1985, 82:1499-1502; and Bruggemann et al., J. Exp. Med., 1987, 166:1351-1361; each of which is incorporated by reference in its entirety. Useful effector cells for such assays include peripheral blood mononuclear cells (PBMC) and Natural Killer (NK) cells. Alternatively, or additionally, ADCC activity of the molecule of interest may be assessed in vivo, using an animal model such as that disclosed in Clynes et al. Proc. Natl. Acad. Sci. U.S.A., 1998, 95:652-656, incorporated by reference in its entirety.

C1q binding assays may also be carried out to confirm that the antibody is unable to bind C1q and hence lacks CDC activity. Examples of C1q binding assays include those described in WO 2006/029879 and WO 2005/100402, each of which is incorporated by reference in its entirety.

Complement activation assays include those described, for example, in Gazzano Santoro et al., J. Immunol. Methods, 1996, 202:163-171; Cragg et al., Blood, 2003, 101:1045-1052; and Cragg and Glennie, Blood, 2004, 103:2738-2743; each of which is incorporated by reference in its entirety.

### 11. Modified Amino Acids

When the antibody conjugate comprises a modified amino acid, the modified amino acid can be any modified amino acid deemed suitable by the practitioner. In particular embodiments, the modified amino acid comprises a reactive group useful for forming a covalent bond to a linker precursor or to a payload precursor. In certain embodiments, the modified amino acid is a non-natural amino acid. In certain embodiments, the reactive group is selected from the group consisting of amino, carboxy, acetyl, hydrazino, hydrazido, semicarbazido, sulfanyl, azido and alkynyl. Modified amino acids are also described in, for example, WO 2013/185115 and WO 2015/006555, each of which is incorporated herein by reference in its entirety.

In certain embodiments, the amino acid residue is according to any of the following formulas:

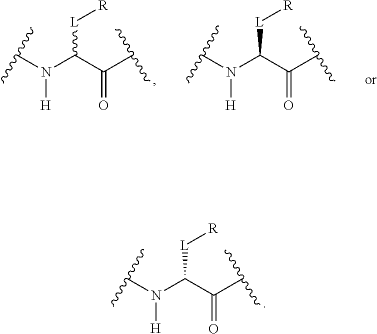

Those of skill in the art will recognize that antibodies are generally comprised of L-amino acids However, with non-natural amino acids, the present methods and compositions provide the practitioner with the ability to use L-, D- or racemic non-natural amino acids at the site-specific positions. In certain embodiments, the non-natural amino acids described herein include D-versions of the natural amino acids and racemic versions of the natural amino acids.

In the above formulas, the wavy lines indicate bonds that connect to the remainder of the polypeptide chains of the antibodies. These non-natural amino acids can be incorporated into polypeptide chains just as natural amino acids are incorporated into the same polypeptide chains. In certain embodiments, the non-natural amino acids are incorporated into the polypeptide chain via amide bonds as indicated in the formulas.

In the above formulas, R designates any functional group without limitation, so long as the amino acid residue is not identical to a natural amino acid residue. In certain embodiments, R can be a hydrophobic group, a hydrophilic group, a polar group, an acidic group, a basic group, a chelating group, a reactive group, a therapeutic moiety or a labeling moiety. In certain embodiments, R is selected from the group consisting of R^{1a}NR^{2a}R^{3a}, R^{1a}C(═O)R^{2a}, R^{1a}C(═O)OR^{2a}, R^{1a}N₃, R^{1a}C(≡CH). In these embodiments, R^{1a} is selected from the group consisting of a bond, alkylene, heteroalkylene, arylene, heteroarylene. R^{2a} and R^{3a} are each independently selected from the group consisting of hydrogen, alkyl and heteroalkyl.

In some embodiments, the non-naturally encoded amino acids include side chain functional groups that react efficiently and selectively with functional groups not found in the 20 common amino acids (including but not limited to, azido, ketone, aldehyde and aminooxy groups) to form stable conjugates. For example, antigen-binding polypeptide that includes a non-naturally encoded amino acid containing an azido functional group can be reacted with a polymer (including but not limited to, poly (ethylene glycol) or, alternatively, a second polypeptide containing an alkyne moiety to form a stable conjugate resulting for the selective reaction of the azide and the alkyne functional groups to form a Huisgen [3+2] cycloaddition product.

Exemplary non-naturally encoded amino acids that may be suitable for use in the present invention and that are useful for reactions with water soluble polymers include, but are not limited to, those with carbonyl, aminooxy, hydrazine, hydrazide, semicarbazide, azide and alkyne reactive groups. In some embodiments, non-naturally encoded amino acids comprise a saccharide moiety. Examples of such amino acids include N-acetyl-L-glucosaminyl-L-serine, N-acetyl-L-galactosaminyl-L-serine, N-acetyl-L-glucosaminyl-L-threonine, N-acetyl-L-glucosaminyl-L-asparagine and O-mannosaminyl-L-serine. Examples of such amino acids also include examples where the naturally-occurring N- or O-linkage between the amino acid and the saccharide is replaced by a covalent linkage not commonly found in nature-including but not limited to, an alkene, an oxime, a thioether, an amide and the like. Examples of such amino acids also include saccharides that are not commonly found in naturally-occurring proteins such as 2-deoxy-glucose, 2-deoxygalactose and the like.

Many of the non-naturally encoded amino acids provided herein are commercially available, e.g., from Sigma-Aldrich (St. Louis, Mo., USA), Novabiochem (a division of EMD Biosciences, Darmstadt, Germany), or Peptech (Burlington, Mass., USA). Those that are not commercially available are optionally synthesized as provided herein or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). See, also, U.S. Patent Application Publications 2003/0082575 and 2003/0108885, which is incorporated by reference herein. In addition to unnatural amino acids that contain unnatural side chains, unnatural amino acids that may be suitable for use in the present invention also optionally comprise modified backbone structures, including but not limited to, as illustrated by the structures of Formula II-1 and III-1:

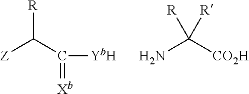

wherein Z typically comprises OH, NH₂, SH, NH—R′, or S—R′; X^{b} and Y^{b}, which can be the same or different, typically comprise S or O, and R and R′, which are optionally the same or different, are typically selected from the same list of constituents for the R group described herein for the unnatural amino acids having Formula I-I as well as hydrogen. For example, unnatural amino acids of the invention optionally comprise substitutions in the amino or carboxyl group as illustrated by Formulas II-I and III-I. Unnatural amino acids of this type include, but are not limited to, α-hydroxy acids, α-thioacids, α-aminothiocarboxylates, including but not limited to, with side chains corresponding to the common twenty natural amino acids or unnatural side chains. In addition, substitutions at the α-carbon optionally include, but are not limited to, L, D, or α-α-disubstituted amino acids such as D-glutamate, D-alanine, D-methyl-O-tyrosine, aminobutyric acid, and the like. Other structural alternatives include cyclic amino acids, such as proline analogues as well as 3, 4, 6, 7, 8, and 9 membered ring proline analogues, P and y amino acids such as substituted β-alanine and γ-amino butyric acid.

Many unnatural amino acids are based on natural amino acids, such as tyrosine, glutamine, phenylalanine, and the like, and are suitable for use in the present invention. Tyrosine analogs include, but are not limited to, para-substituted tyrosines, ortho-substituted tyrosines, and meta substituted tyrosines, where the substituted tyrosine comprises, including but not limited to, a keto group (including but not limited to, an acetyl group), a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, an alkynyl group or the like. In addition, multiply substituted aryl rings are also contemplated. Glutamine analogs that may be suitable for use in the present invention include, but are not limited to, α-hydroxy derivatives, γ-substituted derivatives, cyclic derivatives, and amide substituted glutamine derivatives. Example phenylalanine analogs that may be suitable for use in the present invention include, but are not limited to, para-substituted phenylalanines, ortho-substituted phenyalanines, and meta-substituted phenylalanines, where the substituent comprises, including but not limited to, a hydroxy group, a methoxy group, a methyl group, an allyl group, an aldehyde, an azido, an iodo, a bromo, a keto group (including but not limited to, an acetyl group), a benzoyl, an alkynyl group, or the like. Specific examples of unnatural amino acids that may be suitable for use in the present invention include, but are not limited to, a p-acetyl-L-phenylalanine, an O-methyl-L-tyrosine, an L-3-(2-naphthyl) alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-azido-methyl-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, and a p-propargyloxy-phenylalanine, and the like. Examples of structures of a variety of unnatural amino acids that may be suitable for use in the present invention are provided in, for example, WO 2002/085923 entitled “In vivo incorporation of unnatural amino acids.” See also Kiick et al., (2002) Incorporation of azides into recombinant proteins for chemoselective modification by the Staudinger ligation, PNAS 99:19-24, for additional methionine analogs.

Many of the unnatural amino acids suitable for use in the present invention are commercially available, e.g., from Sigma (USA) or Aldrich (Milwaukee, Wis., USA). Those that are not commercially available are optionally synthesized as provided herein or as provided in various publications or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional publications describing the synthesis of unnatural amino acids include, e.g., WO 2002/085923 entitled “In vivo incorporation of Unnatural Amino Acids;” Matsoukas et al., (1995) J. Med. Chem., 38, 4660-4669; King, F. E. & Kidd, D. A. A. (1949) A New Synthesis of Glutamine and of γ-Dipeptides of Glutamic Acid from Phthylated Intermediates. J. Chem. Soc., 3315-3319; Friedman, O. M. & Chatterrji, R. (1959) Synthesis of Derivatives of Glutamine as Model Substrates for Anti-Tumor Agents. J. Am. Chem. Soc. 81, 3750-3752; Craig, J. C. et al. (1988) Absolute Configuration of the Enantiomers of 7-Chloro-4 [[4-(diethylamino)-1-methylbutyl]amino]quinoline (Chloroquine). J. Org. Chem. 53, 1167-1170; Azoulay, M., Vilmont, M. & Frappier, F. (1991) Glutamine analogues as Potential Antimalarials, Eur. J. Med. Chem. 26, 201-5; Koskinen, A. M. P. & Rapoport, H. (1989) Synthesis of 4-Substituted Prolines as Conformationally Constrained Amino Acid Analogues. J. Org. Chem. 54, 1859-1866; Christie, B. D. & Rapoport, H. (1985) Synthesis of Optically Pure Pipecolates from L-Asparagine. Application to the Total Synthesis of (+)-Apovincamine through Amino Acid Decarbonylation and Iminium Ion Cyclization. J. Org. Chem. 1989:1859-1866; Barton et al., (1987) Synthesis of Novel a-Amino-Acids and Derivatives Using Radical Chemistry: Synthesis of L- and D-a-Amino-Adipic Acids, L-a-aminopimelic Acid and Appropriate Unsaturated Derivatives. Tetrahedron Lett. 43:4297-4308; and, Subasinghe et al., (1992) Quisqualic acid analogues: synthesis of beta-heterocyclic 2-aminopropanoic acid derivatives and their activity at a novel quisqualate-sensitized site. J. Med. Chem. 35:4602-7. See also, patent applications entitled “Protein Arrays,” filed Dec. 22, 2003, Ser. No. 10/744,899 and Ser. No. 60/435,821 filed on Dec. 22, 2002.

Amino acids with a carbonyl reactive group allow for a variety of reactions to link molecules (including but not limited to, PEG or other water soluble molecules) via nucleophilic addition or aldol condensation reactions among others.

Exemplary carbonyl-containing amino acids can be represented as follows:

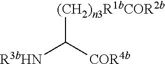

wherein n3 is 0-10; R^{1b} is an alkyl, aryl, substituted alkyl, or substituted aryl; R^{2b} is H, alkyl, aryl, substituted alkyl, and substituted aryl; and R^{3b} is H, an amino acid, a polypeptide, or an amino terminus modification group, and R^{4b} is H, an amino acid, a polypeptide, or a carboxy terminus modification group. In some embodiments, n3 is 1, R^{1b} is phenyl and R^{2b} is a simple alkyl (i.e., methyl, ethyl, or propyl) and the ketone moiety is positioned in the para position relative to the alkyl side chain. In some embodiments, n3 is 1, R^{1b} is phenyl and R^{2b} is a simple alkyl (i.e., methyl, ethyl, or propyl) and the ketone moiety is positioned in the meta position relative to the alkyl side chain.

In some examples, a non-naturally encoded amino acid bearing adjacent hydroxyl and amino groups can be incorporated into the polypeptide as a “masked” aldehyde functionality. For example, 5-hydroxylysine bears a hydroxyl group adjacent to the epsilon amine. Reaction conditions for generating the aldehyde typically involve addition of molar excess of sodium metaperiodate under mild conditions to avoid oxidation at other sites within the polypeptide. The pH of the oxidation reaction is typically about 7.0. A typical reaction involves the addition of about 1.5 molar excess of sodium meta periodate to a buffered solution of the polypeptide, followed by incubation for about 10 minutes in the dark. See, e.g. U.S. Pat. No. 6,423,685, which is incorporated by reference herein.

The carbonyl functionality can be reacted selectively with a hydrazine-, hydrazide-, hydroxylamine-, or semicarbazide-containing reagent under mild conditions in aqueous solution to form the corresponding hydrazone, oxime, or semicarbazone linkages, respectively, that are stable under physiological conditions. See, e.g., Jencks, W. P., J. Am. Chem. Soc. 81, 475-481 (1959); Shao, J. and Tam, J. P., J. Am. Chem. Soc. 117:3893-3899 (1995). Moreover, the unique reactivity of the carbonyl group allows for selective modification in the presence of the other amino acid side chains. See, e.g., Cornish, V. W., et al., J. Am. Chem. Soc. 118:8150-8151 (1996); Geoghegan, K. F. & Stroh, J. G., Bioconjug. Chem. 3:138-146 (1992); Mahal, L. K., et al., Science 276:1125-1128 (1997).

Non-naturally encoded amino acids containing a nucleophilic group, such as a hydrazine, hydrazide or semicarbazide, allow for reaction with a variety of electrophilic groups to form conjugates (including but not limited to, with PEG or other water soluble polymers).

Exemplary hydrazine, hydrazide or semicarbazide-containing amino acids can be represented as follows:

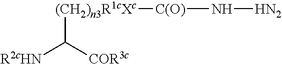

wherein n3 is 0-10; R^{1c} is an alkyl, aryl, substituted alkyl, or substituted aryl or not present; X^{c}, is O, N, or S or not present; R^{2c} is H, an amino acid, a polypeptide, or an amino terminus modification group, and R^{3c} is H, an amino acid, a polypeptide, or a carboxy terminus modification group.

In some embodiments, n3 is 4, R^{1c} is not present, and X is N. In some embodiments, n3 is 2, R^{1c} is not present, and X is not present. In some embodiments, n3 is 1, R^{1c} is phenyl, X is O, and the oxygen atom is positioned para to the aliphatic group on the aryl ring.

Hydrazide-, hydrazine-, and semicarbazide-containing amino acids are available from commercial sources. For instance, L-glutamate-γ-hydrazide is available from Sigma Chemical (St. Louis, Mo.). Other amino acids not available commercially can be prepared by one skilled in the art. See, e.g., U.S. Pat. No. 6,281,211, which is incorporated by reference herein.

Polypeptides containing non-naturally encoded amino acids that bear hydrazide, hydrazine or semicarbazide functionalities can be reacted efficiently and selectively with a variety of molecules that contain aldehydes or other functional groups with similar chemical reactivity. See, e.g., Shao, J. and Tam, J., J. Am. Chem. Soc. 117:3893-3899 (1995). The unique reactivity of hydrazide, hydrazine and semicarbazide functional groups makes them significantly more reactive toward aldehydes, ketones and other electrophilic groups as compared to the nucleophilic groups present on the 20 common amino acids (including but not limited to, the hydroxyl group of serine or threonine or the amino groups of lysine and the N-terminus).

Non-naturally encoded amino acids containing an aminooxy (also called a hydroxylamine) group allow for reaction with a variety of electrophilic groups to form conjugates (including but not limited to, with PEG or other water soluble polymers). Like hydrazines, hydrazides and semicarbazides, the enhanced nucleophilicity of the aminooxy group permits it to react efficiently and selectively with a variety of molecules that contain aldehydes or other functional groups with similar chemical reactivity. See, e.g., Shao, J. and Tam, J., J. Am. Chem. Soc. 117:3893-3899 (1995); H. Hang and C. Bertozzi, Acc. Chem. Res. 34:727-736 (2001). Whereas the result of reaction with a hydrazine group is the corresponding hydrazone, however, an oxime results generally from the reaction of an aminooxy group with a carbonyl-containing group such as a ketone.

Exemplary amino acids containing aminooxy groups can be represented as follows:

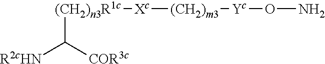

wherein n3 is 0-10; R^{1c} is an alkyl, aryl, substituted alkyl, or substituted aryl or not present; X^{c} is O, N, S or not present; m3 is 0-10; Y^{c} is ═C(O) or not present; R^{2c} is H, an amino acid, a polypeptide, or an amino terminus modification group, and R^{3c} is H, an amino acid, a polypeptide, or a carboxy terminus modification group. In some embodiments, n3 is 1, R^{1a} is phenyl, X^{c} is O, m is 1, and Y^{c} is present. In some embodiments, n3 is 2, R^{1c} and X^{c} are not present, m3 is 0, and Y^{c} is not present.

Aminooxy-containing amino acids can be prepared from readily available amino acid precursors (homoserine, serine and threonine). See, e.g., M. Carrasco and R. Brown, J. Org. Chem. 68:8853-8858 (2003). Certain aminooxy-containing amino acids, such as L-2-amino-4-(aminooxy) butyric acid), have been isolated from natural sources (Rosenthal, G. et al., Life Sci. 60:1635-1641 (1997). Other aminooxy-containing amino acids can be prepared by one skilled in the art.

The unique reactivity of azide and alkyne functional groups makes them extremely useful for the selective modification of polypeptides and other biological molecules. Organic azides, particularly aliphatic azides, and alkynes are generally stable toward common reactive chemical conditions. In particular, both the azide and the alkyne functional groups are inert toward the side chains (i.e., R groups) of the 20 common amino acids found in naturally-occurring polypeptides. When brought into close proximity, however, the “spring-loaded” nature of the azide and alkyne groups is revealed and they react selectively and efficiently via Huisgen [3+2] cycloaddition reaction to generate the corresponding triazole. See, e.g., Chin J., et al., Science 301:964-7 (2003); Wang, Q., et al., J. Am. Chem. Soc. 125, 3192-3193 (2003); Chin, J. W., et al., J. Am. Chem. Soc. 124:9026-9027 (2002).

Because the Huisgen cycloaddition reaction involves a selective cycloaddition reaction (see, e.g., Padwa, A., in COMPREHENSIVE ORGANIC SYNTHESIS, Vol. 4, (ed. Trost, B. M., 1991), p. 1069-1109; Huisgen, R. in 1,3-DIPOLAR CYCLOADDITION CHEMISTRY, (ed. Padwa, A., 1984), p. 1-176) rather than a nucleophilic substitution, the incorporation of non-naturally encoded amino acids bearing azide and alkyne-containing side chains permits the resultant polypeptides to be modified selectively at the position of the non-naturally encoded amino acid. Cycloaddition reaction involving azide or alkyne-containing antibody can be carried out at room temperature under aqueous conditions by the addition of Cu (II) (including but not limited to, in the form of a catalytic amount of CuSO₄) in the presence of a reducing agent for reducing Cu (II) to Cu (I), in situ, in catalytic amount. See, e.g., Wang, Q., et al., J. Am. Chem. Soc. 125, 3192-3193 (2003); Tornoe, C. W., et al., J. Org. Chem. 67:3057-3064 (2002); Rostovtsev, et al., Angew. Chem. Int. Ed. 41:2596-2599 (2002). Exemplary reducing agents include, including but not limited to, ascorbate, metallic copper, quinine, hydroquinone, vitamin K, glutathione, cysteine, Fe²⁺, Co²⁺, and an applied electric potential.

In some cases, where a Huisgen [3+2] cycloaddition reaction between an azide and an alkyne is desired, the antigen-binding polypeptide comprises a non-naturally encoded amino acid comprising an alkyne moiety and the water soluble polymer to be attached to the amino acid comprises an azide moiety. Alternatively, the converse reaction (i.e., with the azide moiety on the amino acid and the alkyne moiety present on the water soluble polymer) can also be performed.

The azide functional group can also be reacted selectively with a water soluble polymer containing an aryl ester and appropriately functionalized with an aryl phosphine moiety to generate an amide linkage. The aryl phosphine group reduces the azide in situ and the resulting amine then reacts efficiently with a proximal ester linkage to generate the corresponding amide. See, e.g., E. Saxon and C. Bertozzi, Science 287, 2007-2010 (2000). The azide-containing amino acid can be either an alkyl azide (including but not limited to, 2-amino-6-azido-1-hexanoic acid) or an aryl azide (p-azido-phenylalanine).

Exemplary water soluble polymers containing an aryl ester and a phosphine moiety can be represented as follows:

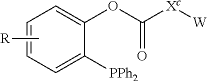

wherein X^{c} can be O, N, S or not present, Ph is phenyl, W is a water soluble polymer and R can be H, alkyl, aryl, substituted alkyl and substituted aryl groups. Exemplary R groups include but are not limited to —CH₂, —C(CH₃)₃, —OR′, —NR′R″, —SR′, -halogen, —C(O)R′, —CONR′R″, —S(O)₂R′, —S(O)₂NR′R″, —CN and —NO₂. R′, R″, R″ and R″″ each independently refer to hydrogen, substituted or unsubstituted heteroalkyl, substituted or unsubstituted aryl, including but not limited to, aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (including but not limited to, —CF₃ and —CH₂CF₃) and acyl (including but not limited to, —C(O)CH₃, —C(O) CF₃, —C(O)CH₂OCH₃, and the like).

The azide functional group can also be reacted selectively with a water soluble polymer containing a thioester and appropriately functionalized with an aryl phosphine moiety to generate an amide linkage. The aryl phosphine group reduces the azide in situ and the resulting amine then reacts efficiently with the thioester linkage to generate the corresponding amide. Exemplary water soluble polymers containing a thioester and a phosphine moiety can be represented as follows:

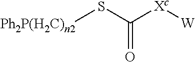

wherein n2 is 1-10; Xc can be O, N, S or not present, Ph is phenyl, and W is a water soluble polymer.

Exemplary alkyne-containing amino acids can be represented as follows:

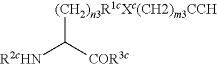

wherein n3 is 0-10; R^{1c} is an alkyl, aryl, substituted alkyl, or substituted aryl or not present; X^{c} is O, N, S or not present; m3 is 0-10, R^{2c} is H, an amino acid, a polypeptide, or an amino terminus modification group, and R^{3c} is H, an amino acid, a polypeptide, or a carboxy terminus modification group. In some embodiments, n3 is 1, R^{1c} is phenyl, X^{c} is not present, m3 is 0 and the acetylene moiety is positioned in the para position relative to the alkyl side chain. In some embodiments, n3 is 1, R^{1c} is phenyl, X^{c} is O, m3 is 1 and the propargyloxy group is positioned in the para position relative to the alkyl side chain (i.e., O-propargyl-tyrosine). In some embodiments, n3 is 1, R^{1c} and X^{c} are not present and m3 is 0 (i.e., proparylglycine).

Alkyne-containing amino acids are commercially available. For example, propargylglycine is commercially available from Peptech (Burlington, Mass.). Alternatively, alkyne-containing amino acids can be prepared according to standard methods. For instance, p-propargyloxyphenylalanine can be synthesized, for example, as described in Deiters, A., et al., J. Am. Chem. Soc. 125:11782-11783 (2003), and 4-alkynyl-L-phenylalanine can be synthesized as described in Kayser, B., et al., Tetrahedron 53 (7): 2475-2484 (1997). Other alkyne-containing amino acids can be prepared by one skilled in the art.

Exemplary azide-containing amino acids can be represented as follows:

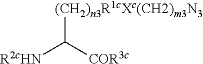

wherein n3 is 0-10; R^{1c} is an alkyl, aryl, substituted alkyl, substituted aryl or not present; X^{c} is O, N, S or not present; m3 is 0-10; R^{2c} is H, an amino acid, a polypeptide, or an amino terminus modification group, and R^{3c} is H, an amino acid, a polypeptide, or a carboxy terminus modification group. In some embodiments, n3 is 1, R^{1c} is phenyl, X^{c} is not present, m3 is 0 and the azide moiety is positioned para to the alkyl side chain. In some embodiments, n3 is 0-4 and R^{1c} and X^{c} are not present, and m3=0. In some embodiments, n3 is 1, R^{1c} is phenyl, X^{c} is O, m3 is 2 and the P-azidoethoxy moiety is positioned in the para position relative to the alkyl side chain.

Azide-containing amino acids are available from commercial sources. For instance, 4-azidophenylalanine can be obtained from Chem-Impex International, Inc. (Wood Dale, Ill.). For those azide-containing amino acids that are not commercially available, the azide group can be prepared relatively readily using standard methods known to those of skill in the art, including but not limited to, via displacement of a suitable leaving group (including but not limited to, halide, mesylate, tosylate) or via opening of a suitably protected lactone. See, e.g., Advanced Organic Chemistry by March (Third Edition, 1985, Wiley and Sons, New York).

The unique reactivity of beta-substituted aminothiol functional groups makes them extremely useful for the selective modification of polypeptides and other biological molecules that contain aldehyde groups via formation of the thiazolidine. See, e.g., J. Shao and J. Tam, J. Am. Chem. Soc. 1995, 117 (14) 3893-3899. In some embodiments, beta-substituted aminothiol amino acids can be incorporated into antibodies and then reacted with water soluble polymers comprising an aldehyde functionality. In some embodiments, a water soluble polymer, drug conjugate or other payload can be coupled to an antibody polypeptide comprising a beta-substituted aminothiol amino acid via formation of the thiazolidine.

Particular examples of useful non-natural amino acids include, but are not limited to, p-acetyl-L-phenylalanine, O-methyl-L-tyrosine, L-3-(2-naphthyl) alanine, 3-methyl-phenylalanine, O-4-allyl-L-tyrosine, 4-propyl-L-tyrosine, tri-O-acetyl-GlcNAc b-serine, L-Dopa, fluorinated phenylalanine, isopropyl-L-phenylalanine, p-azido-methyl-L-phenylalanine, p-azido-L-phenylalanine, p-acyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, and p-propargyloxy-phenylalanine. Further useful examples include N-acetyl-L-glucosaminyl-L-serine, N-acetyl-L-galactosaminyl-L-serine, N-acetyl-L-glucosaminyl-L-threonine, N-acetyl-L-glucosaminyl-L-asparagine and O-mannosaminyl-L-serine.

In particular embodiments, the non-natural amino acids are selected from p-acetyl-phenylalanine, p-ethynyl-phenylalanine, p-propargyloxyphenylalanine, p-azido-methyl-phenylalanine, and p-azido-phenylalanine. One particularly useful non-natural amino acid is p-azido phenylalanine. This amino acid residue is known to those of skill in the art to facilitate Huisgen [3+2] cyloaddition reactions (so-called “click” chemistry reactions) with, for example, compounds bearing alkynyl groups. This reaction enables one of skill in the art to readily and rapidly conjugate to the antibody at the site-specific location of the non-natural amino acid.

In certain embodiments, the first reactive group is an alkynyl moiety (including but not limited to, in the unnatural amino acid p-propargyloxyphenylalanine, where the propargyl group is also sometimes referred to as an acetylene moiety) and the second reactive group is an azido moiety, and [3+2] cycloaddition chemistry can be used. In certain embodiments, the first reactive group is the azido moiety (including but not limited to, in the unnatural amino acid p-azido-L-phenylalanine) and the second reactive group is the alkynyl moiety.

In the above formulas, each L represents a divalent linker. The divalent linker can be any divalent linker known to those of skill in the art. Generally, the divalent linker is capable of forming covalent bonds to the functional moiety R and the cognate reactive group (e.g., alpha carbon) of the non-natural amino acid. Useful divalent linkers a bond, alkylene, substituted alkylene, heteroalkylene, substituted heteroalkylene, arylene, substituted arylene, heteroarlyene and substituted heteroarylene. In certain embodiments, L is C₁₋₁₀ alkylene or C₁₋₁₀ heteroalkylene.

The non-natural amino acids used in the methods and compositions described herein have at least one of the following four properties: (1) at least one functional group on the sidechain of the non-natural amino acid has at least one characteristics and/or activity and/or reactivity orthogonal to the chemical reactivity of the 20 common, genetically-encoded amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), or at least orthogonal to the chemical reactivity of the naturally occurring amino acids present in the polypeptide that includes the non-natural amino acid; (2) the introduced non-natural amino acids are substantially chemically inert toward the 20 common, genetically-encoded amino acids; (3) the non-natural amino acid can be stably incorporated into a polypeptide, preferably with the stability commensurate with the naturally-occurring amino acids or under typical physiological conditions, and further preferably such incorporation can occur via an in vivo system; and (4) the non-natural amino acid includes an oxime functional group or a functional group that can be transformed into an oxime group by reacting with a reagent, preferably under conditions that do not destroy the biological properties of the polypeptide that includes the non-natural amino acid (unless of course such a destruction of biological properties is the purpose of the modification/transformation), or where the transformation can occur under aqueous conditions at a pH between about 4 and about 8, or where the reactive site on the non-natural amino acid is an electrophilic site. Any number of non-natural amino acids can be introduced into the polypeptide. Non-natural amino acids may also include protected or masked oximes or protected or masked groups that can be transformed into an oxime group after deprotection of the protected group or unmasking of the masked group. Non-natural amino acids may also include protected or masked carbonyl or dicarbonyl groups, which can be transformed into a carbonyl or dicarbonyl group after deprotection of the protected group or unmasking of the masked group and thereby are available to react with hydroxylamines or oximes to form oxime groups.

In further embodiments, non-natural amino acids that may be used in the methods and compositions described herein include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or non-covalently interact with other molecules, photocaged and/or photoisomerizable amino acids, amino acids comprising biotin or a biotin analogue, glycosylated amino acids such as a sugar substituted serine, other carbohydrate modified amino acids, keto-containing amino acids, aldehyde-containing amino acids, amino acids comprising polyethylene glycol or other polyethers, heavy atom substituted amino acids, chemically cleavable and/or photocleavable amino acids, amino acids with an elongated side chains as compared to natural amino acids, including but not limited to, polyethers or long chain hydrocarbons, including but not limited to, greater than about 5 or greater than about 10 carbons, carbon-linked sugar-containing amino acids, redox-active amino acids, amino thioacid containing amino acids, and amino acids comprising one or more toxic moiety.

In some embodiments, non-natural amino acids comprise a saccharide moiety. Examples of such amino acids include N-acetyl-L-glucosaminyl-L-serine, N-acetyl-L-galactosaminyl-L-serine, N-acetyl-L-glucosaminyl-L-threonine, N-acetyl-L-glucosaminyl-L-asparagine and O-mannosaminyl-L-serine. Examples of such amino acids also include examples where the naturally-occurring N- or O-linkage between the amino acid and the saccharide is replaced by a covalent linkage not commonly found in nature-including but not limited to, an alkene, an oxime, a thioether, an amide and the like. Examples of such amino acids also include saccharides that are not commonly found in naturally-occurring proteins such as 2-deoxy-glucose, 2-deoxygalactose and the like.

The chemical moieties incorporated into antibodies via incorporation of non-natural amino acids offer a variety of advantages and manipulations of polypeptides. For example, the unique reactivity of a carbonyl or dicarbonyl functional group (including a keto- or aldehyde-functional group) allows selective modification of antibodies with any of a number of hydrazine- or hydroxylamine-containing reagents in vivo and in vitro. A heavy atom non-natural amino acid, for example, can be useful for phasing x-ray structure data. The site-specific introduction of heavy atoms using non-natural amino acids also provides selectivity and flexibility in choosing positions for heavy atoms. Photoreactive non-natural amino acids (including but not limited to, amino acids with benzophenone and arylazides (including but not limited to, phenylazide) side chains), for example, allow for efficient in vivo and in vitro photocrosslinking of polypeptides. Examples of photoreactive non-natural amino acids include, but are not limited to, p-azido-phenylalanine and p-benzoyl-phenylalanine. The antibodies with the photoreactive non-natural amino acids may then be crosslinked at will by excitation of the photoreactive group-providing temporal control. In a non-limiting example, the methyl group of a non-natural amino can be substituted with an isotopically labeled, including but not limited to, with a methyl group, as a probe of local structure and dynamics, including but not limited to, with the use of nuclear magnetic resonance and vibrational spectroscopy.

Amino acids with an electrophilic reactive group allow for a variety of reactions to link molecules via various chemical reactions, including, but not limited to, nucleophilic addition reactions. Such electrophilic reactive groups include a carbonyl- or dicarbonyl-group (including a keto- or aldehyde group), a carbonyl-like- or dicarbonyl-like-group (which has reactivity similar to a carbonyl- or dicarbonyl-group and is structurally similar to a carbonyl- or dicarbonyl-group), a masked carbonyl- or masked dicarbonyl-group (which can be readily converted into a carbonyl- or dicarbonyl-group), or a protected carbonyl- or protected dicarbonyl-group (which has reactivity similar to a carbonyl- or dicarbonyl-group upon deprotection). Such amino acids include amino acids having the structure of Formula (I-1):

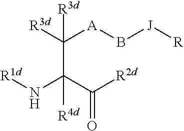

wherein: A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene; B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-,
   —S—, —S-(alkylene or substituted alkylene)-, —S(O)ₖ— where k is 1, 2, or 3, —S(O) (alkylene or substituted alkylene)-, —C(O)—, —NS(O)₂—, —OS(O)₂—, —C(O)-(alkylene or substituted alkylene)-,
   —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl; J is

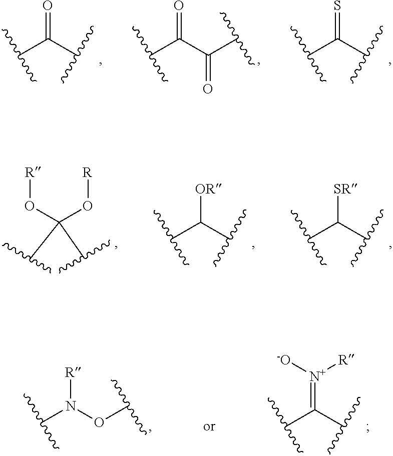

R is H, alkyl, substituted alkyl, cycloalkyl, or substituted cycloalkyl; each R″ is independently H, alkyl, substituted alkyl, or a protecting group, or when more than one R″ group is present, two R″ optionally form a heterocycloalkyl; R^{1d} is H, an amino protecting group, resin, amino acid, polypeptide, or polynucleotide; and R^{2d} is OH, an ester protecting group, resin, amino acid, polypeptide, or polynucleotide; each of R^{3d} and R^{4d} is independently H, halogen, lower alkyl, or substituted lower alkyl, or R^{3d} and R^{4d} or two R^{3d} groups optionally form a cycloalkyl or a heterocycloalkyl; or the -A-B-J-R groups together form a bicyclic or tricyclic cycloalkyl or heterocycloalkyl comprising at least one carbonyl group, including a dicarbonyl group, protected carbonyl group, including a protected dicarbonyl group, or masked carbonyl group, including a masked dicarbonyl group; or the -J-R group together forms a monocyclic or bicyclic cycloalkyl or heterocycloalkyl comprising at least one carbonyl group, including a dicarbonyl group, protected carbonyl group, including a protected dicarbonyl group, or masked carbonyl group, including a masked dicarbonyl group; with a proviso that when A is phenylene and each R^{3d} is H, B is present; and that when A is —(CH₂)₄— and each R^{3d} is H, B is not —NHC(O)(CH₂CH₂)—; and that when A and B are absent and each R^{3d} is H, R is not methyl. Such non-natural amino acids may be in the form of a salt, or may be incorporated into a non-natural amino acid polypeptide, polymer, polysaccharide, or a polynucleotide and optionally post translationally modified.

In certain embodiments, compounds of Formula (I-1) are stable in aqueous solution for at least 1 month under mildly acidic conditions. In certain embodiments, compounds of Formula (I-1) are stable for at least 2 weeks under mildly acidic conditions. In certain embodiments, compounds of Formula (I-1) are stable for at least 5 days under mildly acidic conditions. In certain embodiments, such acidic conditions are pH 2 to 8.

In certain embodiments of compounds of Formula (I-1), B is lower alkylene, substituted lower alkylene, —O-(alkylene or substituted alkylene)-, —C(R′)═N—N(R′)—, —N(R′)CO—, —C(O)—, —C(R′)═N—, —C(O)-(alkylene or substituted alkylene)-, —CON(R′)-(alkylene or substituted alkylene)-, —S(alkylene or substituted alkylene)-, —S(O) (alkylene or substituted alkylene)-, or —S(O)₂ (alkylene or substituted alkylene)-. In certain embodiments of compounds of Formula (I-1), B is —O(CH₂)—, —CH═N—, —CH═N—NH—, —NHCH₂—, —NHCO—, —C(O)—, —C(O)—(CH₂)—, —CONH—(CH₂)—, —SCH₂—, —S(═O)CH₂—, or —S(O)₂CH₂—. In certain embodiments of compounds of Formula (I-1), R is C₁₋₆ alkyl or cycloalkyl. In certain embodiments of compounds of Formula (I-1) R is —CH₃, —CH(CH₃)₂, or cyclopropyl. In certain embodiments of compounds of Formula (I-1), R^{1d} is H, tert-butyloxycarbonyl (Boc), 9-Fluorenylmethoxycarbonyl (Fmoc), N-acetyl, tetrafluoroacetyl (TFA), or benzyloxycarbonyl (Cbz). In certain embodiments of compounds of Formula (I-1), R^{1d} is a resin, amino acid, polypeptide, or polynucleotide. In certain embodiments of compounds of Formula (I-1), R^{2d} is OH, O-methyl, O-ethyl, or O-t-butyl. In certain embodiments of compounds of Formula (I-1), R^{2d} is a resin, amino acid, polypeptide, or polynucleotide. In certain embodiments of compounds of Formula (I-1), R^{2d} is a polynucleotide. In certain embodiments of compounds of Formula (I-1), R^{2d} is ribonucleic acid (RNA). In certain embodiments of compounds of Formula (I-1), R^{2d} is tRNA. In certain embodiments of compounds of Formula (I-1), the tRNA specifically recognizes a selector codon. In certain embodiments of compounds of Formula (I-1) the selector codon is selected from the group consisting of an amber codon, ochre codon, opal codon, a unique codon, a rare codon, an unnatural codon, a five-base codon, and a four-base codon. In certain embodiments of compounds of Formula (I-1), R^{2d} is a suppressor tRNA.

In certain embodiments of compounds of Formula (I-2),

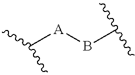

is selected from the group consisting of: (i) A is substituted lower alkylene, C₄-arylene, substituted arylene, heteroarylene, substituted heteroarylene, alkarylene, substituted alkarylene, aralkylene, or substituted aralkylene; B is optional, and when present is a divalent linker selected from the group consisting of lower alkylene, substituted lower alkylene, lower alkenylene, substituted lower alkenylene, —O—, —O-(alkylene or substituted alkylene)-, —S—, —S(O)—, —S(O)₂—, —NS (O)₂—, —OS(O)₂—, —C(O)—, —C(O)-(alkylene or substituted alkylene)-, —C(S)—, —N(R′)—, —C(O)N(R′)—, —CON(R′)-(alkylene or substituted alkylene)-, —CSN(R′)—, —N(R′)CO-(alkylene or substituted alkylene)-, —N(R′)C(O)O—, —N(R′)C(S)—, —S(O)N(R′), —S(O)₂N(R′), —N(R′) C(O)N(R′)—, —N(R′)C(S)N(R′)—, —N(R′)S(O)N(R′)—, —N(R′)S(O)₂N(R′)—, —N(R′)—N═, —C(R′)—N—N(R′)—, —C(R′)═N—N═, —C(R′)₂—N═N—, and —C(R′)₂—N(R′)—N(R′)—; (ii) A is optional, and when present is substituted lower alkylene, C₄-arylene, substituted arylene, heteroarylene, substituted heteroarylene, alkarylene, substituted alkarylene, aralkylene, or substituted aralkylene; B is a divalent linker selected from the group consisting of lower alkylene, substituted lower alkylene, lower alkenylene, substituted lower alkenylene, —O—, —O-(alkylene or substituted alkylene)-, —S—, —S(O)—, —S(O)₂—, —NS(O)₂—, —OS(O)₂—, —C(O)—, —C(O)-(alkylene or substituted alkylene)-, —C(S)—, —N(R′)—, —C(O)N(R′)—, —CON(R′)-(alkylene or substituted alkylene)-, —CSN(R′)—, —N(R′)CO-(alkylene or substituted alkylene)-, —N(R′)C(O)O—, —N(R′)C(S)—, —S(O)N(R′), —S(O)₂N(R′), —N(R′)C(O)N(R′)—, —N(R′)C(S)N(R′)—, —N(R′)S(O)N(R′)—, —N(R′)S(O)₂N(R′)—, —N(R′)—N═, —C(R′)═N—N(R′)—, —C(R′)═N—N═, —C(R′)₂—N═N—, and —C(R′)₂—N(R′)—N(R′)—; (iii) A is lower alkylene; B is optional, and when present is a divalent linker selected from the group consisting of lower alkylene, substituted lower alkylene, lower alkenylene, substituted lower alkenylene, —O—, —O-(alkylene or substituted alkylene)-, —S—, —S(O)—, —S(O)₂—, —NS(O)₂—, —OS(O)₂—, —C(O)—, —C(O)-(alkylene or substituted alkylene)-, —C(S)—, —N(R′)—, —C(O)N(R′)—, —CSN(R′)—, —CON(R′)-(alkylene or substituted alkylene)-, —N(R′)C(O)O—, —N(R′) C(S)—, —S(O)N(R′), —S(O)₂N(R′), —N(R′)C(O)N(R′)—, —N(R′)C(S)N(R′)—, —N(R′)S(O)N(R′)—, —N(R′)S(O)₂N(R′)—, —N(R′)—N═, —C(R′)═N—N(R′)—, —C(R′)═N—N═, —C(R′)₂—N═N—, and —C(R′)₂—N(R′)—N(R′)—; and (iv) A is phenylene; B is a divalent linker selected from the group consisting of lower alkylene, substituted lower alkylene, lower alkenylene, substituted lower alkenylene, —O—, —O-(alkylene or substituted alkylene)-, —S—, —S(O)—, —S(O)₂—, —NS(O)₂—, —OS(O)₂—, —C(O)—, —C(O)-(alkylene or substituted alkylene)-, —C(S)—, —N(R′)—, —C(O)N(R′)—, —CON(R′)-(alkylene or substituted alkylene)-, —CSN(R′)—, —N(R′)CO-(alkylene or substituted alkylene)-, —N(R′)C(O)O—, —N(R′)C(S)—, —S(O)N(R′), —S(O)₂N(R′), —N(R′)C(O)N(R′)—, —N(R′)C(S)N(R′)—, —N(R′)S(O)N(R′)—, —N(R′)S(O)₂N(R′)—, —N(R′)—N═, —C(R′)′N—N(R′)—, —C(R′)═N—N═, —C(R′)₂—N═N—, and —C(R′)₂—N(R′)—N(R′)—; J is

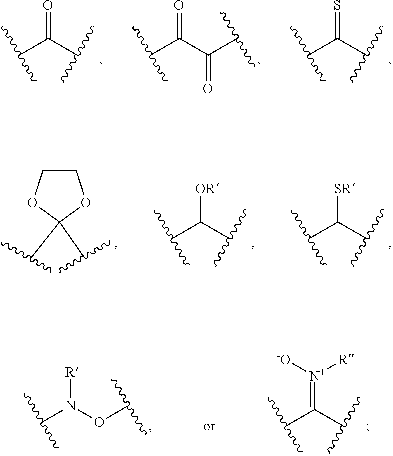

each R′ is independently H, alkyl, or substituted alkyl; R^{1d} is optional, and when present, is H, an amino protecting group, resin, amino acid, polypeptide, or polynucleotide; and R^{2d} is optional, and when present, is OH, an ester protecting group, resin, amino acid, polypeptide, or polynucleotide; and each R^{3d} and R^{4d} is independently H, halogen, lower alkyl, or substituted lower alkyl; and R is H, alkyl, substituted alkyl, cycloalkyl, or substituted cycloalkyl.

In certain embodiments, the non-natural amino acid can be according to formula XIX:

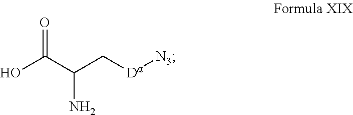

or a salt thereof, wherein: D^{a} is —Ar—W^{3a}— or —W^{1a}—Y^{1a}—C(O)—Y^{2a}—W^{2a}—; Ar is

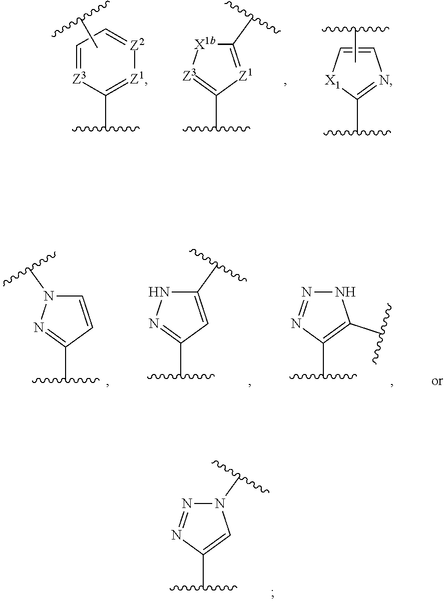

each of W^{1a}, W^{2a}, and W^{3a} is independently a single bond or lower alkylene; each X^{1b} is independently —NH—, —O—, or —S—; each Y^{1a} is independently a single bond, —NH—, or —O—; each Y^{2a} is independently a single bond, —NH—, —O—, or an N-linked or C-linked pyrrolidinylene; and one of Z¹, Z², and Z³ is —N— and the others of Z¹, Z², and Z³ are independently —CH—. In certain embodiments, the non-natural amino acid is according to formula XIXa:

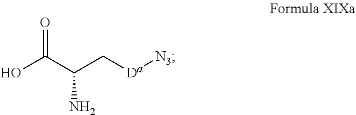

where D^{a} is a defined in the context of formula XIX. In certain embodiments, the non-natural amino acid is according formula XIXb:

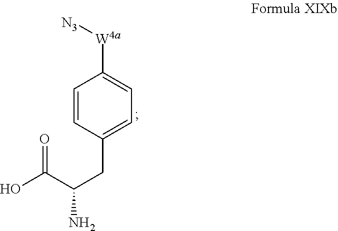

or a salt thereof, wherein W^{4a} is C₁-C₁₀ alkylene. In a further embodiment, W^{4a} is C₁-C₅ alkylene. In an embodiment, W^{4a} is C₁-C₃ alkylene. In an embodiment, W^{4a} is C₁ alkylene. In particular embodiments, the non-natural amino acid is selected from the group consisting of:

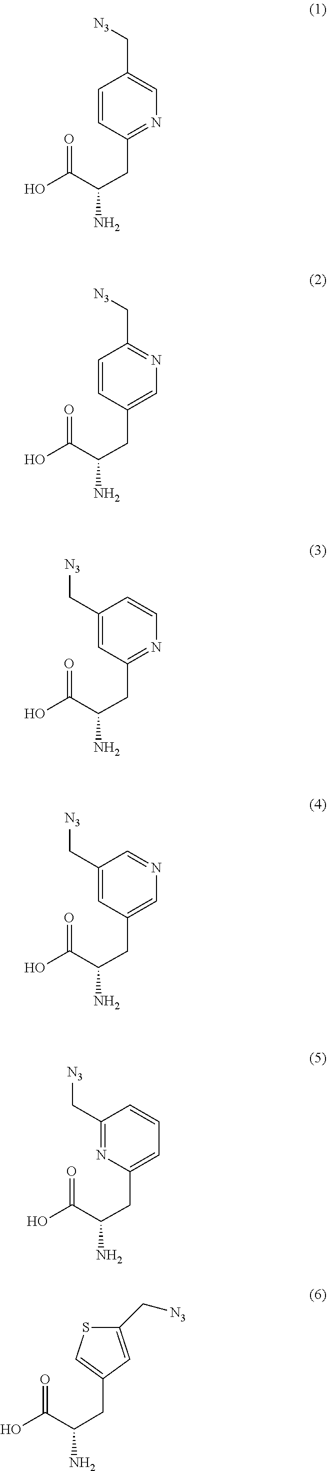 [SMILES: [N-]=[N+]=NCc1ccc(C[C@H](N)C(=O)O)nc1 ; [N-]=[N+]=NCc1ccc(C[C@H](N)C(=O)O)cn1 ; [N-]=[N+]=NCc1ccnc(C[C@H](N)C(=O)O)c1 ; [N-]=[N+]=NCc1cncc(C[C@H](N)C(=O)O)c1 ; [N-]=[N+]=NCc1cccc(C[C@H](N)C(=O)O)n1 ; [N-]=[N+]=NCc1cc(C[C@H](N)C(=O)O)cs1]

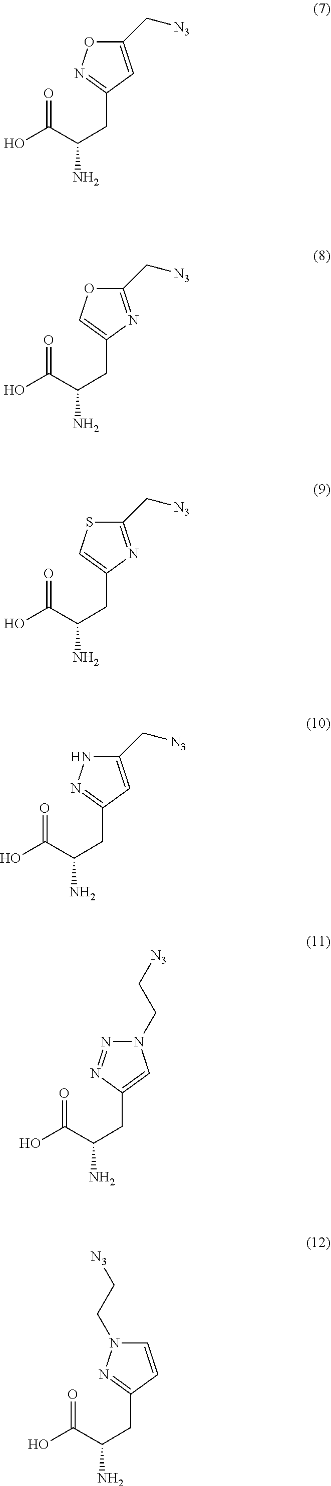 [SMILES: [N-]=[N+]=NCc1cc(C[C@H](N)C(=O)O)no1 ; [N-]=[N+]=NCc1nc(C[C@H](N)C(=O)O)co1 ; [N-]=[N+]=NCc1nc(C[C@H](N)C(=O)O)cs1 ; [N-]=[N+]=NCc1cc(C[C@H](N)C(=O)O)n[nH]1 ; [N-]=[N+]=NCCn1cc(C[C@H](N)C(=O)O)nn1 ; [N-]=[N+]=NCCn1ccc(C[C@H](N)C(=O)O)n1]

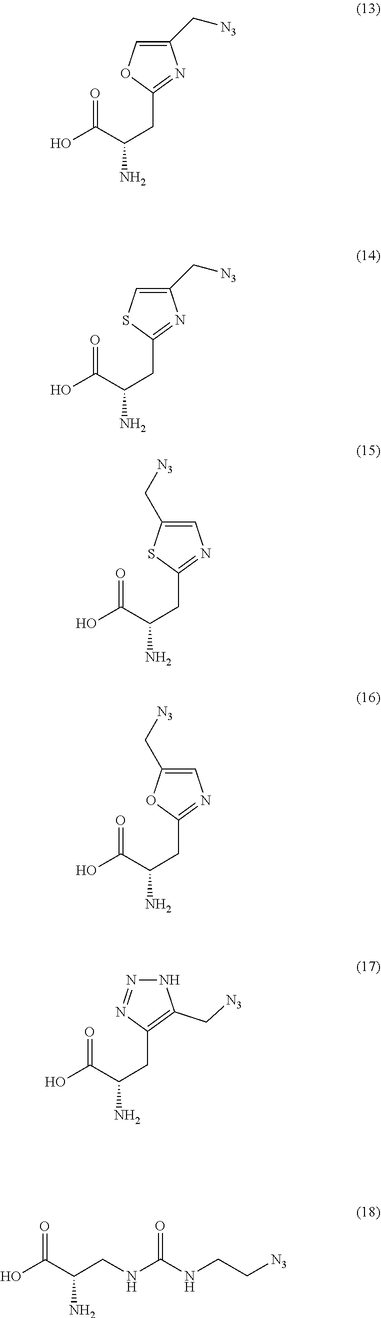 [SMILES: [N-]=[N+]=NCc1coc(C[C@H](N)C(=O)O)n1 ; [N-]=[N+]=NCc1csc(C[C@H](N)C(=O)O)n1 ; [N-]=[N+]=NCc1cnc(C[C@H](N)C(=O)O)s1 ; [N-]=[N+]=NCc1cnc(C[C@H](N)C(=O)O)o1 ; [N-]=[N+]=NCc1[nH]nnc1C[C@H](N)C(=O)O ; [N-]=[N+]=NCCNC(=O)NC[C@H](N)C(=O)O]

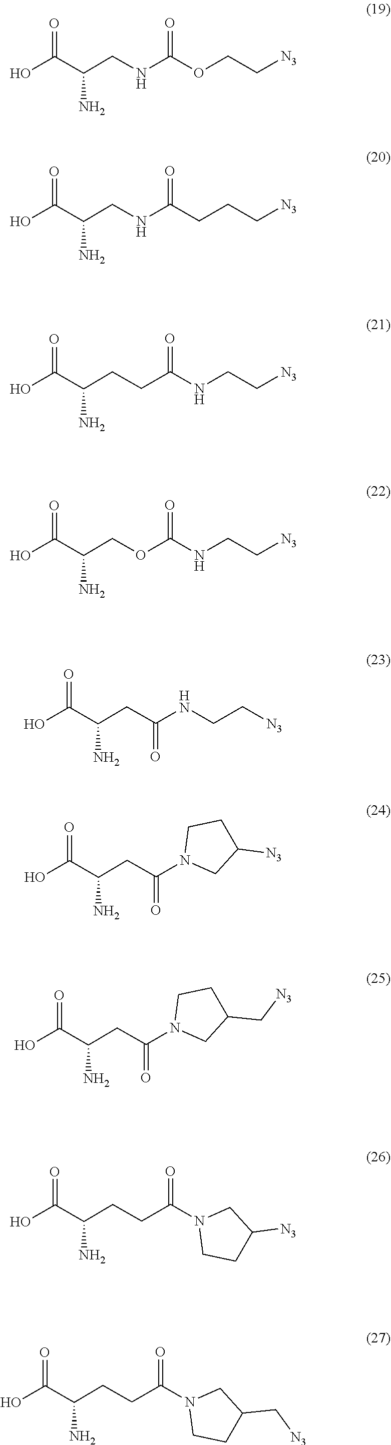

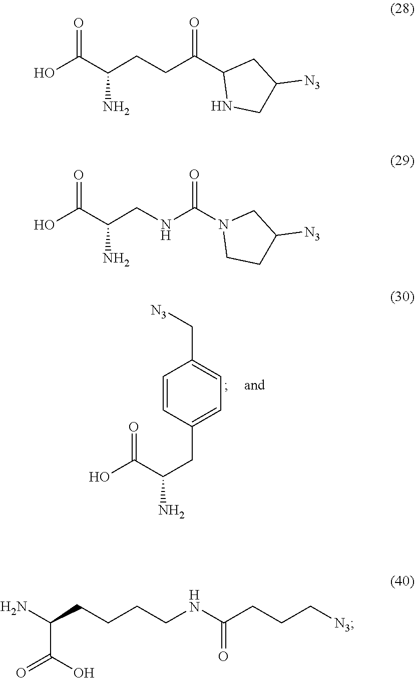 [SMILES: [N-]=[N+]=NC1CNC(C(=O)CC[C@H](N)C(=O)O)C1 ; [N-]=[N+]=NC1CCN(C(=O)NC[C@H](N)C(=O)O)C1 ; [N-]=[N+]=NCc1ccc(C[C@H](N)C(=O)O)cc1 ; [N-]=[N+]=NCCCC(=O)NCCCC[C@H](N)C(=O)O]

or a salt thereof. Such non-natural amino acids may be in the form of a salt, or may be incorporated into a non-natural amino acid polypeptide, polymer, polysaccharide, or a polynucleotide and optionally post translationally modified.

In certain embodiments, the modified amino acid is according to formula I:

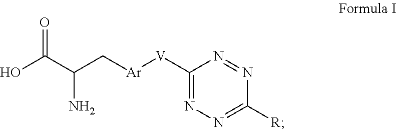

or a salt thereof, wherein Ar is:

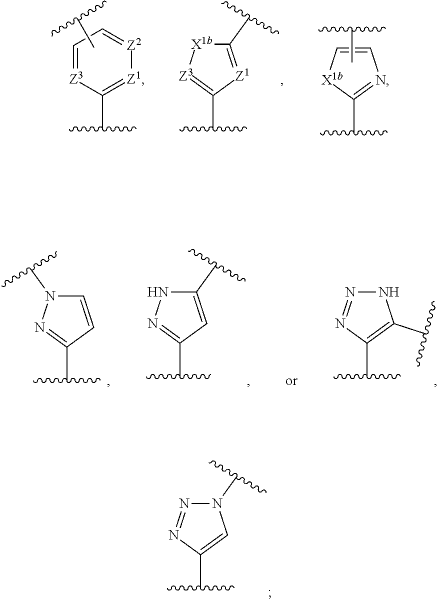

V is a single bond, lower alkylene, or —W^{1a}-W^{2a}-; one of W^{1a} and W^{2a} is absent or lower alkylene, and the other is —NH—, —O—, or —S—; each X^{1b} is independently —NH—, —O—, or —S—; one of Z¹, Z², and Z³ is —CH— or —N— and the others of Z¹, Z², and Z³ are each independently —CH—; and R is lower alkyl. In certain embodiments, when Ar is

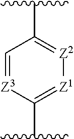

and V is —NH—, then one of Z¹, Z², and Z³ is —N—. In certain embodiments, V is a single bond, —NH—, or —CH₂NH—

In certain embodiments, Ar is

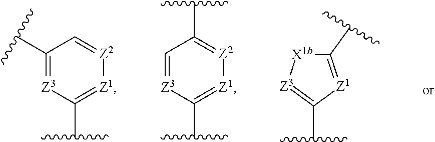

and Z¹, Z², Z³ and X^{1b} are as defined in the context of formula I. In certain embodiments according to this paragraph, V is —W^{1a}-W^{2a}-; one of W^{1a} and W^{2a} is absent or —CH₂—, and the other is —NH—, —O—, or —S—. In certain embodiments according to this paragraph, V is a single bond, —NH—, or —CH₂NH—. In certain embodiments according to this paragraph, Z¹ is N. In certain embodiments according to this paragraph, Z² is N. In certain embodiments according to this paragraph, Z³ is N. In certain embodiments according to this paragraph, Z¹ is CH, Z³ is CH and X^{1b} is S.

In certain embodiments, Ar is

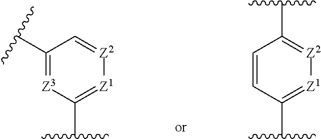

and Z¹, Z², and Z³ are as defined in the context of formula I. In certain embodiments according to this paragraph, V is —W^{1a}-W^{2a}-; one of W^{1a} and W^{2a} is absent or —CH₂—, and the other is —NH—, —O—, or —S—. In certain embodiments according to this paragraph, V is a single bond, —NH—, or —CH₂NH—. In certain embodiments according to this paragraph, Z¹ is N. In certain embodiments according to this paragraph, Z² is N. In certain embodiments according to this paragraph, Z³ is N.

In certain embodiments, Ar is

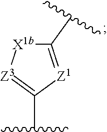

and Z¹, Z³ and X¹ are as defined in the context of Formula I-1. In certain embodiments according to this paragraph, V is —W^{1a}-W^{2a}-; one of W^{1a} and W^{2a} is absent or —CH₂—, and the other is —NH—, —O—, or —S—. In certain embodiments according to this paragraph, V is a single bond, —NH—, or —CH₂NH—. In certain embodiments according to this paragraph, Z¹ is N. In certain embodiments according to this paragraph, Z³ is N. In certain embodiments according to this paragraph, Z¹ is CH, Z³ is CH and X^{1b} is S.

In certain embodiments, the modified amino acid is according to Formula Ia:

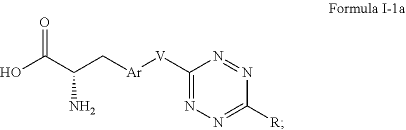

where Ar, V, and R are defined in the context of Formula I-1.

In an embodiment, compounds of either of Formulas I-1 and I-1a are provided wherein V is a single bond. In another embodiment, compounds of either of Formulas I-1 and I-1a are provided wherein V is —NH—. In another embodiment, compounds of either of Formulas I-1 and I-1a are provided wherein V is —CH₂NH—.

In certain embodiments, the modified amino acid is according to Formula II-1:

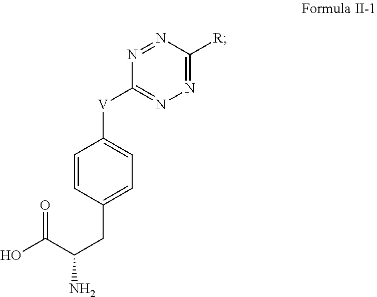

or a salt thereof, wherein V and R are as defined in Formula I-1. In certain embodiments according to this paragraph, V is —W^{1a}-W^{2a}-; one of W^{1a} and W^{2a} is absent or —CH₂—, and the other is —NH—, —O—, or —S—. In certain embodiments, V is a single bond, —NH—, or —CH₂NH—. In certain embodiments, V is a single bond or —CH₂NH—; and R is methyl.

In certain embodiments, the modified amino acid is according to Formula III-1:

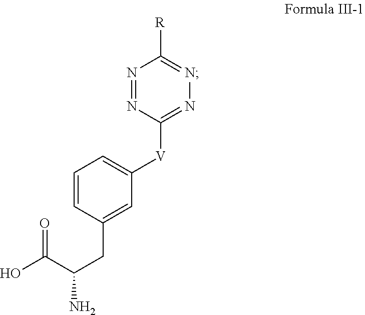

or a salt thereof, wherein V and R are as defined in Formula I-1. In certain embodiments according to this paragraph, V is —W^{1a}-W^{2a}-; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to Formula IV-1:

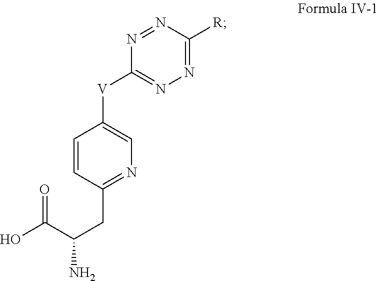

or a salt thereof, wherein V and R are as defined in Formula I-1. In certain embodiments according to this paragraph, V is —W^{1a}-W^{2a}-; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to Formula V-1:

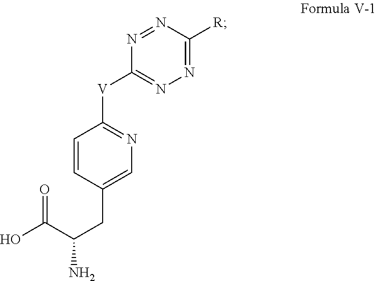

or a salt thereof, wherein V and R are as defined in Formula I. In certain embodiments according to this paragraph, V is —W^{1a}-W^{2a}-; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to Formula VI-1:

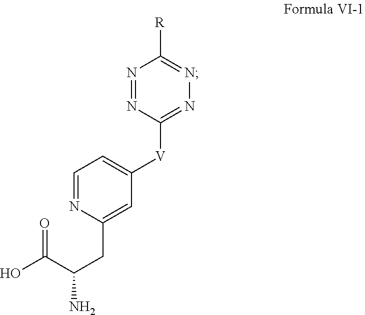

or a salt thereof, wherein V and R are as defined in Formula I. In certain embodiments according to this paragraph, V is —W^{1a}-W^{2a}-; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to Formula VII-1:

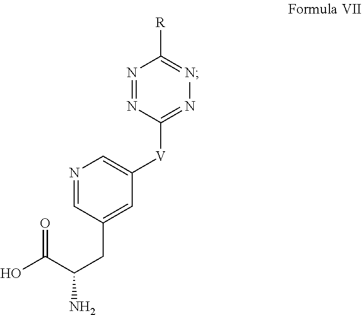

or a salt thereof, wherein V and R are as defined in Formula I. In certain embodiments according to this paragraph, V is —W^{1a}-W^{2a}-; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to Formula VIII-1:

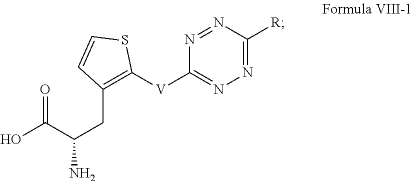

Or a salt thereof, wherein V and R are as defined in Formula I. In certain embodiments according to this paragraph, V is —W^{1a}-W^{2a}; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to Formula IX-1:

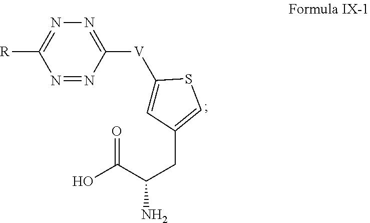

or a salt thereof, wherein V and R are as defined in Formula I-1. In certain embodiments according to this paragraph, V is —W^{1a}-W^{2a}-; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to any of formulas 51-62:

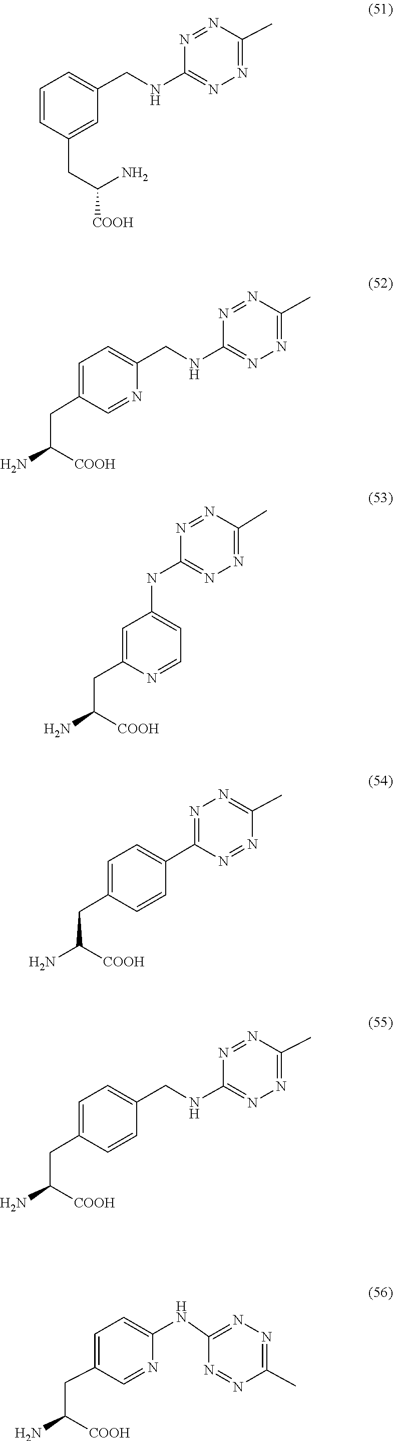

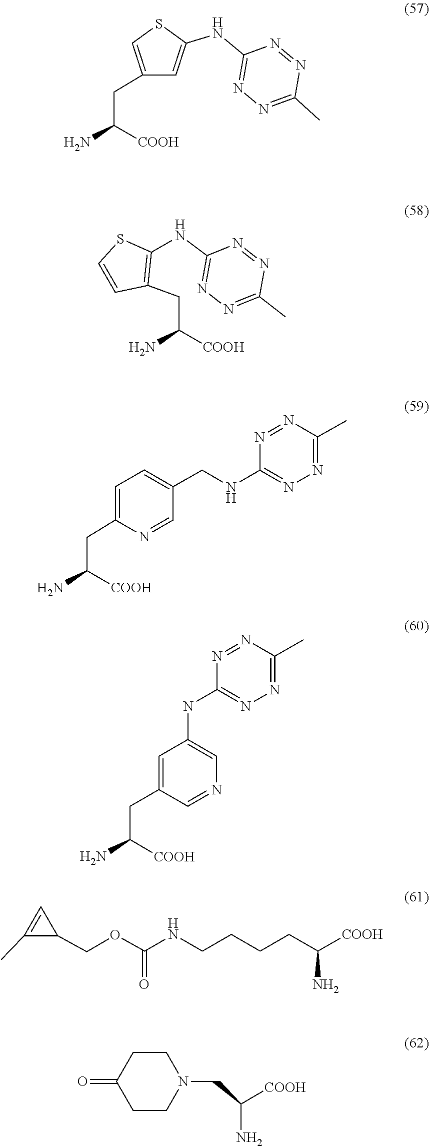

or a salt thereof.

In certain embodiments, the non-natural amino acid is selected from the group consisting of compounds 30, 53, 56, 59, 60, 61, and 62 above. In certain embodiments, the non-natural amino acid is compound 30. In certain embodiments, the non-natural amino acid is compound 56. In some embodiments, the non-natural amino acid is compound 61. In some embodiments, the non-natural amino acid is compound 62.

### 12. Preparation of Antibodies and Antibody Conjugates

### 12.1. Antigen Preparation

The tissue factor antigen to be used for isolation of the antibodies may be intact tissue factor or a fragment of tissue factor. The intact tissue factor, or fragment of tissue factor, may be in the form of an isolated protein or protein expressed by a cell. Other forms of tissue factor useful for generating antibodies will be apparent to those skilled in the art.

### 12.2. Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, CA, incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

### 12.3. Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370; each of which is incorporated by reference in its entirety.

### 12.4. Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety).

### 12.5 Conjugation

The antibody conjugates can be prepared by standard techniques. In certain embodiments, an antibody is contacted with a payload precursor under conditions suitable for forming a bond from the antibody to the payload to form an antibody-payload conjugate. In certain embodiments, an antibody is contacted with a linker precursor under conditions suitable for forming a bond from the antibody to the linker. The resulting antibody-linker is contacted with a payload precursor under conditions suitable for forming a bond from the antibody-linker to the payload to form an antibody-linker-payload conjugate. In certain embodiments, a payload precursor is contacted with a linker precursor under conditions suitable for forming a bond from the payload to the linker. The resulting payload-linker is contacted with an antibody under conditions suitable for forming a bond from the payload-linker to the antibody to form an antibody-linker-payload conjugate. Suitable linkers for preparing the antibody conjugates are disclosed herein, and exemplary conditions for conjugation are described in the Examples below.

In some embodiments, an anti-Tissue Factor conjugate is prepared by contacting an anti-Tissue Factor antibody as disclosed herein with a linker precursor having a structure of any of LP1-LP5:

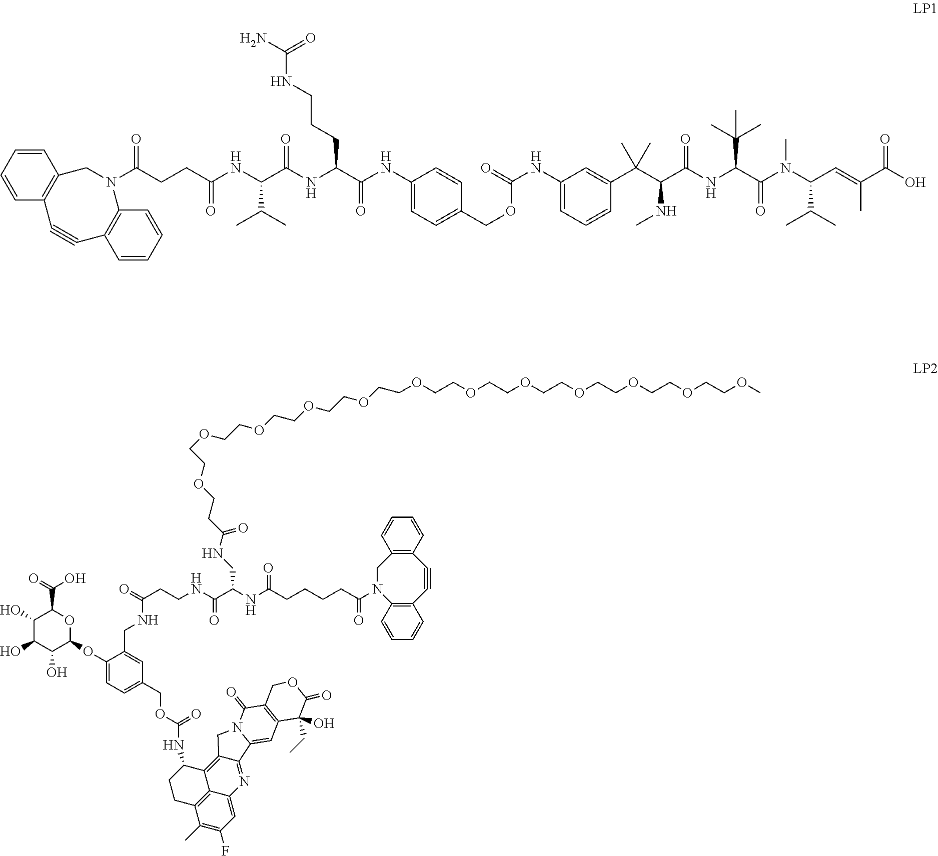 [SMILES: CN[C@H](C(=O)N[C@H](C(=O)N(C)[C@H](/C=C(\C)C(=O)O)C(C)C)C(C)(C)C)C(C)(C)c1cccc(NC(=O)OCc2ccc(NC(=O)[C@H](CCCNC(N)=O)NC(=O)[C@@H](NC(=O)CCC(=O)N3Cc4ccccc4C#Cc4ccccc43)C(C)C)cc2)c1 ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CNC(=O)CCNC(=O)[C@H](CNC(=O)CCOCCOCCOCCOCCOCCOCCOCCOCCOCCOCCOCCOC)NC(=O)CCCCC(=O)N2Cc4ccccc4C#Cc4ccccc42)c1)CC3]

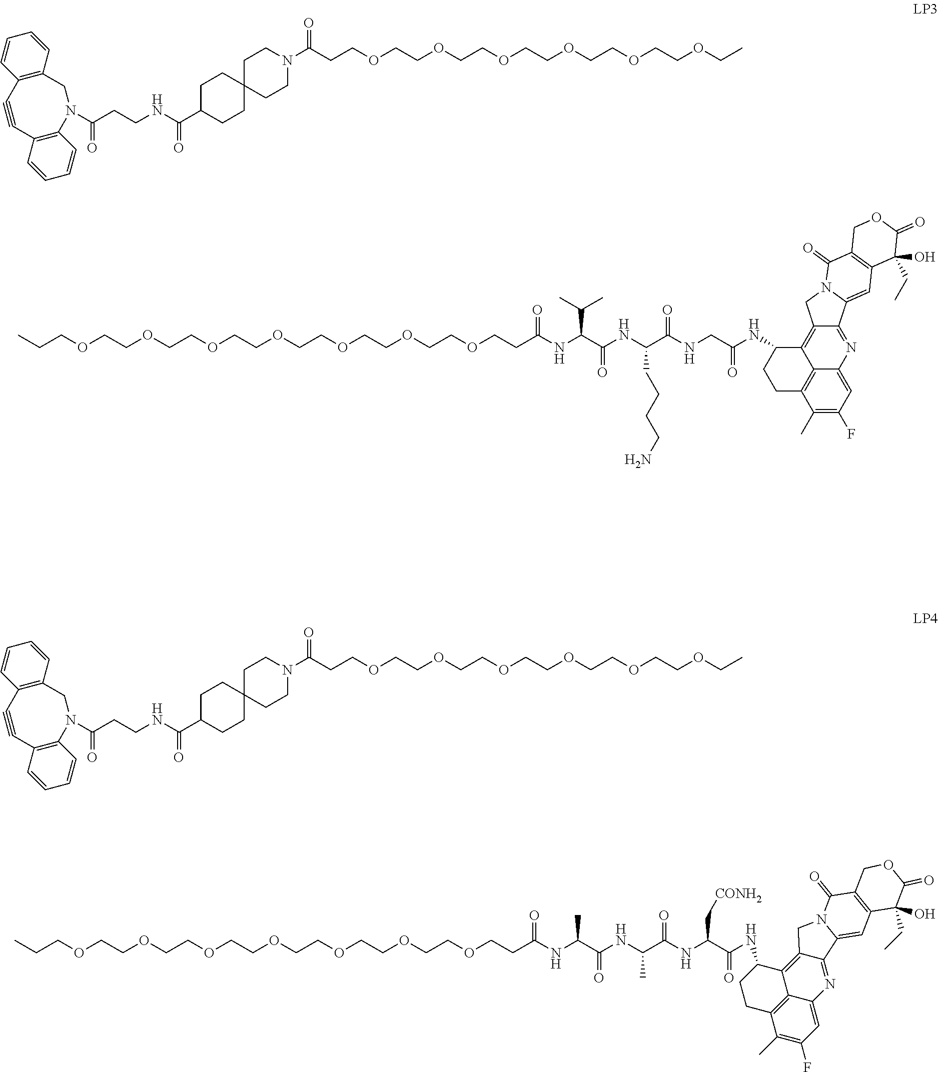

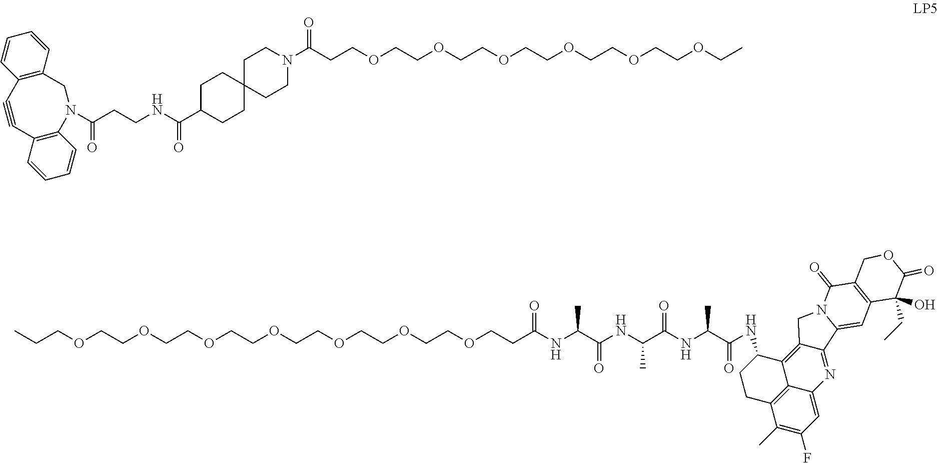

i.

In some embodiments, an anti-Tissue Factor conjugate is prepared by contacting an anti-Tissue Factor antibody as disclosed herein with a linker precursor having a structure of any of LP1A-LP5A:

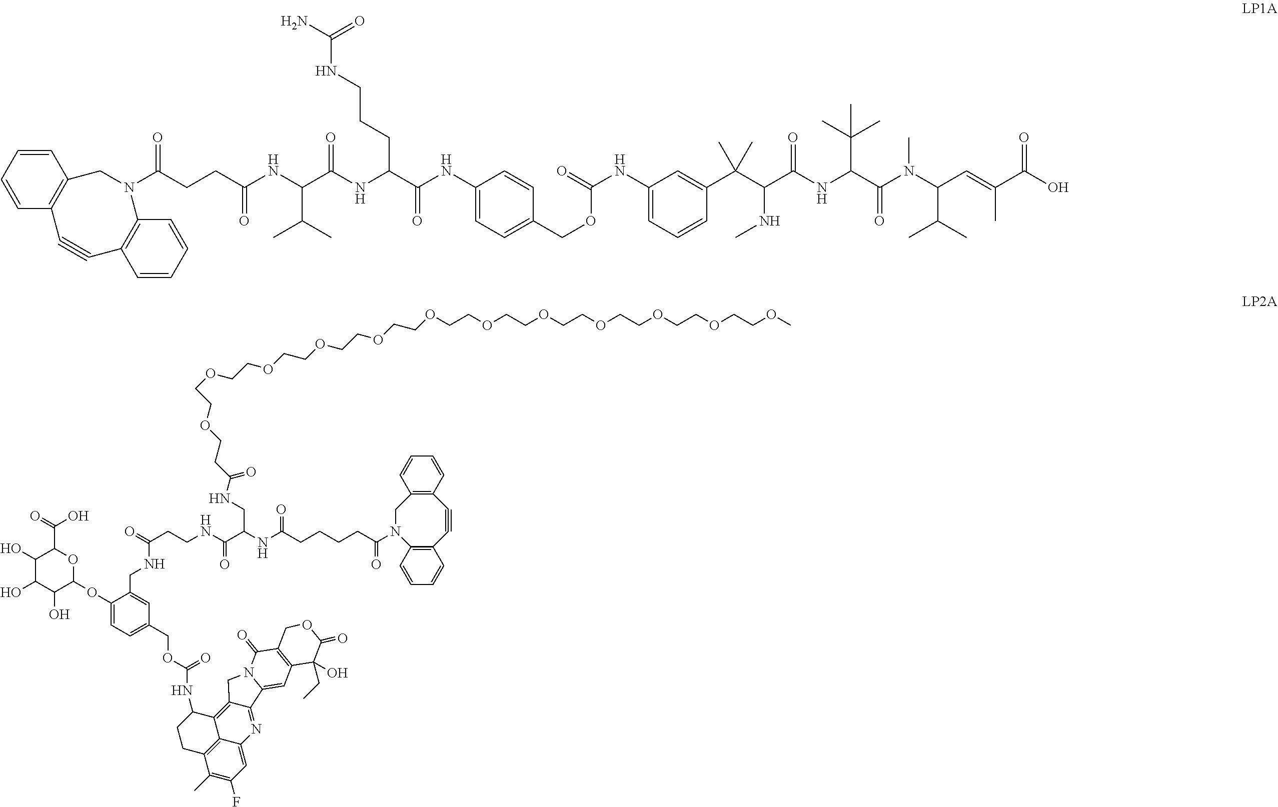 [SMILES: CNC(C(=O)NC(C(=O)N(C)C(/C=C(\C)C(=O)O)C(C)C)C(C)(C)C)C(C)(C)c1cccc(NC(=O)OCc2ccc(NC(=O)C(CCCNC(N)=O)NC(=O)C(NC(=O)CCC(=O)N3Cc4ccccc4C#Cc4ccccc43)C(C)C)cc2)c1 ; CCC1(O)C(=O)OCc2c1cc1n(c2=O)Cc2c-1nc1cc(F)c(C)c3c1c2C(NC(=O)OCc1ccc(OC2OC(C(=O)O)C(O)C(O)C2O)c(CNC(=O)CCNC(=O)C(CNC(=O)CCOCCOCCOCCOCCOCCOCCOCCOCCOCCOCCOCCOC)NC(=O)CCCCC(=O)N2Cc4ccccc4C#Cc4ccccc42)c1)CC3]

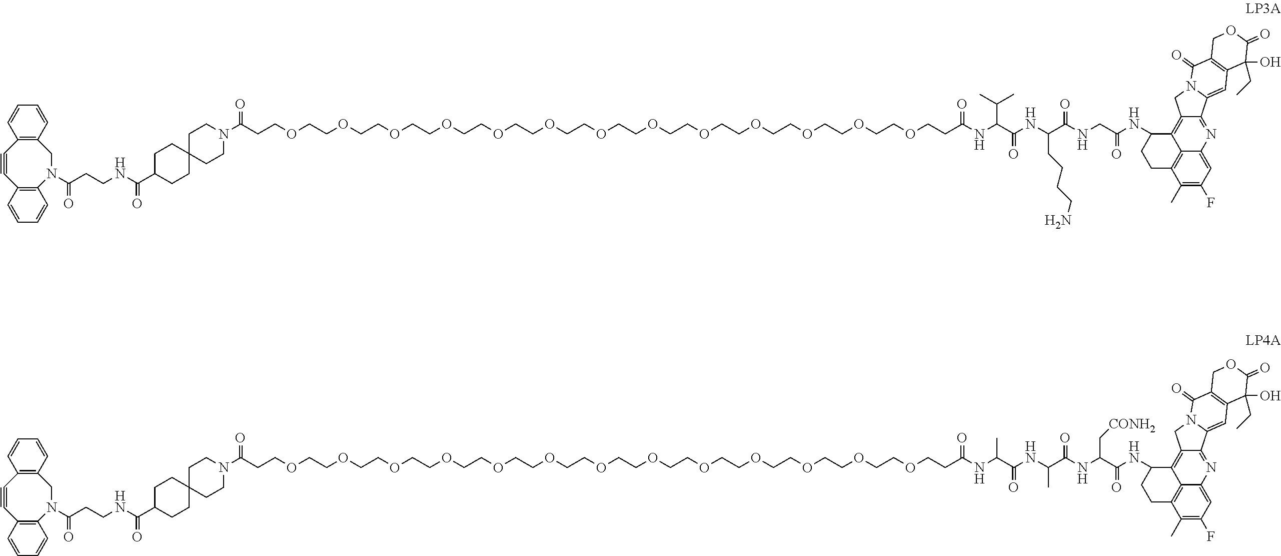 [SMILES: CCC1(O)C(=O)OCc2c1cc1n(c2=O)Cc2c-1nc1cc(F)c(C)c3c1c2C(NC(=O)CNC(=O)C(CCCCN)NC(=O)C(NC(=O)CCOCCOCCOCCOCCOCCOCCOCCOCCOCCOCCOCCOCCOCCC(=O)N1CCC2(CCC(C(=O)NCCC(=O)N4Cc5ccccc5C#Cc5ccccc54)CC2)CC1)C(C)C)CC3 ; CCC1(O)C(=O)OCc2c1cc1n(c2=O)Cc2c-1nc1cc(F)c(C)c3c1c2C(NC(=O)C(CC(N)=O)NC(=O)C(C)NC(=O)C(C)NC(=O)CCOCCOCCOCCOCCOCCOCCOCCOCCOCCOCCOCCOCCOCCC(=O)N1CCC2(CCC(C(=O)NCCC(=O)N4Cc5ccccc5C#Cc5ccccc54)CC2)CC1)CC3]

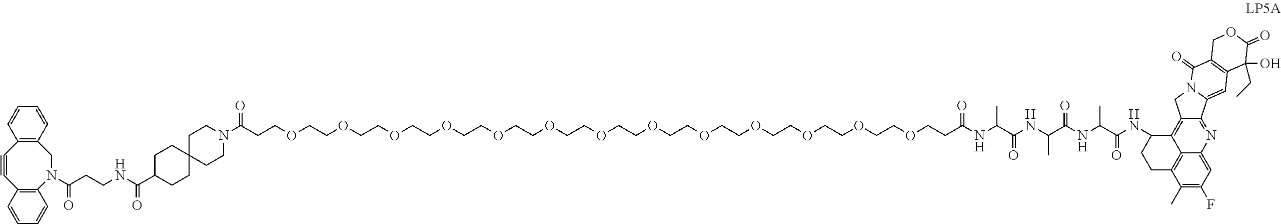 [SMILES: CCC1(O)C(=O)OCc2c1cc1n(c2=O)Cc2c-1nc1cc(F)c(C)c3c1c2C(NC(=O)C(C)NC(=O)C(C)NC(=O)C(C)NC(=O)CCOCCOCCOCCOCCOCCOCCOCCOCCOCCOCCOCCOCCOCCC(=O)N1CCC2(CCC(C(=O)NCCC(=O)N4Cc5ccccc5C#Cc5ccccc54)CC2)CC1)CC3]

### 13. Vectors, Host Cells, and Recombinant Methods

The invention also provides isolated nucleic acids encoding anti-tissue factor antibodies, vectors and host cells comprising the nucleic acids, and recombinant techniques for the production of the antibodies.

For recombinant production of the antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W311LP0 are suitable.

In addition to prokaryotes, eukaryotic microbes such as filamentous fungi or yeast are also suitable cloning or expression hosts for anti-tissue factor antibody-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the anti-tissue factor antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio/Technology, 1992, 10:163-167) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly (styrenedivinyl) benzene allow for faster flow rates and shorter processing times than can be achieved with agarose. Where the antibody comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### 14. Pharmaceutical Compositions and Methods of Administration

The antibodies or the antibody conjugates provided herein can be formulated into pharmaceutical compositions using methods available in the art and those disclosed herein. Any of the antibodies or the antibody conjugates provided herein can be provided in any appropriate pharmaceutical composition and be administered by any suitable route of administration. Suitable routes of administration include, but are not limited to, the inhalation, intraarterial, intradermal, intramuscular, intraperitoneal, intravenous, nasal, parenteral, pulmonary, and subcutaneous routes.

The methods provided herein encompass administering pharmaceutical compositions comprising at least one antibody or antibody conjugate provided herein and one or more compatible and pharmaceutically acceptable carriers. In this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Martin, E.W., Remington's Pharmaceutical Sciences.

In clinical practice the pharmaceutical compositions, antibodies, or antibody conjugates provided herein may be administered by any route known in the art. Exemplary routes of administration include, but are not limited to, the inhalation, intraarterial, intradermal, intramuscular, intraperitoneal, intravenous, nasal, parenteral, pulmonary, and subcutaneous routes. In some embodiments, a pharmaceutical composition, antibody, or antibody conjugate provided herein is administered parenterally.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing and stabilizing agents. Sterilization can be carried out in several ways, for example using a bacteriological filter, by radiation or by heating. Parenteral compositions can also be prepared in the form of sterile solid compositions which can be dissolved at the time of use in sterile water or any other injectable sterile medium.

In some embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibody conjugates.

The pharmaceutical composition may comprise one or more pharmaceutical excipients. Any suitable pharmaceutical excipient may be used, and one of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific antibody in the dosage form. The composition or single unit dosage form, if desired, can also contain minor amounts of wetting or emulsifying agents, or pH buffering agents. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6^{th} Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a co-solvent. Illustrative examples of co-solvents include ethanol, poly (ethylene) glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene (glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6^{th} Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an antibody or antibody conjugate, since, in some embodiments, water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Lactose-free compositions provided herein can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre gelatinized starch, and magnesium stearate.

Also provided are pharmaceutical compositions and dosage forms that comprise one or more excipients that reduce the rate by which an antibody or antibody-conjugate will decompose. Such excipients, which are referred to herein as “stabilizers,” include, but are not limited to, antioxidants such as ascorbic acid, pH buffers, or salt buffers.

### 14.1. Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

### 14.2. Dosage and Unit Dosage Forms

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies or antibody conjugates.

The amount of the antibody or antibody conjugate or composition which will be effective in the prevention or treatment of a disorder or one or more symptoms thereof will vary with the nature and severity of the disease or condition, and the route by which the antibody or antibody conjugate is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the antibody or antibody conjugate per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiment, the dosage of the antibody or antibody conjugate provided herein, based on weight of the antibody or antibody conjugate, administered to prevent, treat, manage, or ameliorate a disorder, or one or more symptoms thereof in a subject is between 0.1 mg/kg and 15 mg/kg of a subject's body weight. In another embodiment, the dosage of the composition or a composition provided herein administered to prevent, treat, manage, or ameliorate a disorder, or one or more symptoms thereof in a subject is between 0.1 mg and 200 mg.

The dose can be administered according to a suitable schedule. It may be necessary to use dosages of the antibody outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an antibody or composition provided herein followed by one or more maintenance doses.

In certain embodiments, a dose of an antibody or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

### 14.3 Combination Therapies and Formulations

In certain embodiments, provided are methods for treatment and/or compositions and therapeutic formulations comprising any of the antibodies or antibody conjugates provided herein in combination with one or more chemotherapeutic agents disclosed herein, and methods of treatment comprising administering such combinations to subjects in need thereof. Examples of chemotherapeutic agents include, but are not limited to, Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially uncialamycin, calicheamicin gammall, and calicheamicin omegall (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pladienolide B, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

In certain embodiments, provided are methods for treatment and/or compositions and therapeutic formulations comprising any of the antibodies or antibody conjugates provided herein in combination with a checkpoint inhibitor, for example, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or other inhibitor.

In certain embodiments, provided are methods for treatment and/or compositions and therapeutic formulations comprising any of the antibodies or antibody conjugates provided herein in combination with one or more PD-1 or PD-L1 inhibitors, and methods of treatment comprising administering such combinations to subjects in need thereof. In some embodiments, the one or more PD-1 or PD-L1 inhibitors comprise a small molecule blocker of the PD-1 or PD-L1 pathway. In some embodiments, the one or more PD-1 or PD-L1 inhibitors comprise an antibody that inhibits PD-1 or PD-L1 activity. In some embodiments, the one or more PD-1 or PD-L1 inhibitors are selected from the group consisting of: CA-170, BMS-8, BMS-202, BMS-936558, CK-301, and AUNP12. In some embodiments, the one or more PD-1 or PD-L1 inhibitors are selected from the group consisting of: avelumab, nivolumab, pembrolizumab, atezolizumab, durvalumab, AMP-224 (GlaxoSmithKline), MEDI0680/AMP-514 (AstraZeneca), PDR001 (Novartis), cemiplimab, TSR-042 (Tesaro), Tizlelizumab/BGB-A317 (Beigene), CK-301 (Checkpoint Therapeutics), BMS-936559 (Bristol-Meyers Squibb), camrelizumab, sintilimab, toripalimab, genolimzumab, and A167 (Sichuan Kelun-Biotech Biopharmaceutical). In some embodiments, the one or more PD-1 or PD-L1 inhibitors are selected from the group consisting of: MGA012 (Incyte/MacroGenics), PF-06801591 (Pfizer/Merck KGaA), LY3300054 (Eli Lilly), FAZ053 (Novartis), PD-11 (Novartis), CX-072 (CytomX), BGB-A333 (Beigene), BI 754091 (Boehringer Ingelheim), JNJ-63723283 (Johnson and Johnson/Jannsen), AGEN2034 (Agenus), CA-327 (Curis), CX-188 (CytomX), STI-A1110 (Servier), JTX-4014 (Jounce), (LLY) AM0001 (Armo Biosciences), CBT-502 (CBT Pharmaceuticals), FS118 (F-Star/Merck KGaA), XmAb20717 (Xencor), XmAb23104 (Xencor), AB122 (Arcus Biosciences), KY1003 (Kymab), RXI-762 (RXi). In some embodiments, the one or more PD-1 or PD-L1 inhibitors are selected from the group consisting of: PRS-332 (Pieris Pharmaceuticals), ALPN-202 (Alpine Immune Science), TSR-075 (Tesaro/Anaptys Bio), MCLA-145 (Merus), MGD013 (Macrogenics), MGD019 (Macrogenics). An additional PD-1 inhibitor is Pidilizumab (CT-011). In some embodiments, the one or more PD-1 or PD-L1 inhibitors are selected from an anti-PD1 mono-specific or bi-specific antibody described in, for example, WO 2016/077397, WO 2018/156777, and International Application No. PCT/US2013/034213, filed May 23, 2018.

In some embodiments, the one or more PD-1 or PD-L1 inhibitors are selected from the group consisting of CA-170, BMS-8, BMS-202, BMS-936558, CK-301, AUNP12, avelumab, nivolumab, pembrolizumab, atezolizumab, durvalumab, AMP-224, MEDI0680/AMP-514, PDR001, cemiplimab, TSR-042, Tizlelizumab/BGB-A317, CK-301, BMS-936559, camrelizumab, sintilimab, toripalimab, genolimzumab, A167, MGA012, PF-06801591, LY3300054, FAZ053, PD-11, CX-072, BGB-A333, BI 754091, JNJ-63723283, AGEN2034, CA-327, CX-188, STI-A1110, JTX-4014, (LLY) AM0001, CBT-502, FS118, XmAb20717, XmAb23104, AB122, KY1003, RXI-762, PRS-33, ALPN-202, TSR-075, MCLA-145, MGD013, and MGD019.

In certain embodiments, provided are methods for treatment and/or compositions and therapeutic formulations comprising any of the antibodies or antibody conjugates provided herein in combination with one or more CTLA-4 inhibitors, and methods of treatment comprising administering such combinations to subjects in need thereof. CTLA-4 inhibitors include, but are not limited to, ipilimumab (Yervoy®), tremelimumab (AstraZeneca and MedImmune), AGEN1884 and AGEN2041 (Agenus).

In certain embodiments, provided are methods for treatment and/or compositions and therapeutic formulations comprising any of the antibodies or antibody conjugates provided herein in combination with one or more LAG-3 inhibitors, and methods of treatment comprising administering such combinations to subjects in need thereof. Examples of LAG-3 immune checkpoint inhibitors include, but are not limited to, BMS-986016 (Bristol-Myers Squibb), GSK2831781 (GaxoSmithKline), IMP321 (Prima BioMed), LAG525 (Novartis), and the dual PD-1 and LAG-3 inhibitor MGD013 (MacroGenics).

In certain embodiments, provided are methods for treatment and/or compositions and therapeutic formulations comprising any of the antibodies or antibody conjugates provided herein in combination with one or more TIM-3 inhibitors, and methods of treatment comprising administering such combinations to subjects in need thereof. A specific TIM-3 inhibitor includes, but is not limited to, TSR-022 (Tesaro).

Other immune checkpoint inhibitors for use in the invention described herein include, but are not limited to, B7-H3/CD276 immune checkpoint inhibitors such as MGA217; indoleamine 2,3-dioxygenase (IDO) immune checkpoint inhibitors such as Indoximod and INCB024360; killer immunoglobulin-like receptors (KIRs) immune checkpoint inhibitors such as Lirilumab (BMS-986015); and, carcinoembryonic antigen cell adhesion molecule (CEACAM) inhibitors (e.g., CEACAM-1, -3 and/or -5).

In certain embodiments, provided are methods for treatment and/or compositions or therapeutic formulations comprising any of the antibody conjugates provided herein in combination with a PARP inhibitor, including, but not limited to olaparib (AZD-2281, Lynparza®), rucaparib (AG 014699, Rubraca®), niraparib (Zejula®), talazoparib (BMN-673, Talzenna®), veliparib (ABT-888), fluzoparib (HS10160), Iniparib (BSI 201), BGB-290, E7016, E7449, and CEP-9722. In one embodiment, an antibody conjugate provided herein is administered in combination with olaparib, rucaparib, niraparib, or talazoparib. In one embodiment, an antibody conjugate provided herein is administered in combination with olaparib.

In certain embodiments, provided are methods for treatment and/or composition or therapeutic formulations comprising any of the antibody conjugates provided herein in combination with an ATR inhibitor, ATM inhibitor, Wee1 inhibitor, CHK1 inhibitor, NAMPT inhibitor, or a Topo2 inhibitor.

The agents administered in combination with the antibodies or antibody conjugates disclosed herein can be administered just prior to, sequentially, concurrent with, or shortly after the administration of the antibodies or antibody conjugates. In one embodiment, the agents administered in combination with the antibodies or antibody conjugates disclosed herein are administered sequentially after the administration of the antibody conjugates. In one embodiment, the agents administered in combination with the antibodies or antibody conjugates disclosed herein are administered concurrently after the administration of the antibody conjugates. For purposes of the present disclosure, such administration regimens are considered the administration of an antibody conjugate “in combination with” an additional therapeutically active component. Embodiments include pharmaceutical compositions in which an antibody conjugate disclosed herein is co-formulated with one or more of the chemotherapeutic agents, PD-1 inhibitors, PD-L1 inhibitors, or PARP inhibitors disclosed herein.

### 15. Therapeutic Applications

For therapeutic applications, the antibodies or antibody conjugates of the invention are administered to a mammal, generally a human, in a pharmaceutically acceptable dosage form such as those known in the art and those discussed above. For example, the antibodies or antibody conjugates of the invention may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The antibodies or antibody conjugates also are suitably administered by peritumoral, intralesional, or perilesional routes, to exert local as well as systemic therapeutic effects. The intraperitoneal route may be particularly useful, for example, in the treatment of ovarian tumors.

In some aspects, a method for reducing cell proliferation in a subject in need thereof can comprise administering to the subject an effective amount of an isolated antibody, antibody conjugate or pharmaceutical composition thereof of the present disclosure. In some aspects, an isolated antibody, antibody conjugate or pharmaceutical composition thereof of the present disclosure can be used for reducing cell proliferation in a subject in need thereof. In some aspects, an isolated antibody, antibody conjugate or pharmaceutical composition thereof of the present disclosure can be used for the preparation of a medicament for reducing cell proliferation in a subject in need thereof.

In some aspects, a method for treating or preventing a disease or condition in a subject in need thereof can comprise administering to the subject an effective amount of an isolated antibody, antibody conjugate or pharmaceutical composition thereof of the present disclosure. In some aspects, an isolated antibody, antibody conjugate or pharmaceutical composition thereof of the present disclosure can be used for treating or preventing a disease or condition in a subject in need thereof. In some aspects, an isolated antibody, antibody conjugate or pharmaceutical composition thereof of the present disclosure can be used for the preparation of a medicament for treating or preventing a disease or condition in a subject in need thereof.

The antibodies or antibody conjugates provided herein may be useful for the treatment of any disease or condition involving tissue factor. In some embodiments, the disease or condition is a disease or condition that can be diagnosed by overexpression of tissue factor. In some embodiments, the disease or condition is a disease or condition that can benefit from treatment with an anti-tissue factor antibody or antibody conjugate. In some embodiments, the disease or condition is cancer. In some embodiments, the disease or condition is a Tissue Factor-expressing cancer. In certain embodiments, the anti-TF antibody conjugates treat the disease or condition, for example cancer, by inducing DNA damage and/or cell death, or by activating anti-tumor immunity or protective immunity.

Any suitable cancer may be treated with the antibodies or antibody conjugates provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, head and neck squamous cell carcinoma, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In some embodiments, the disease to be treated with the antibodies or antibody conjugates provided herein is gastric cancer, colorectal cancer, renal cell carcinoma, cervical carcinoma, endometrial cancer, non-small cell lung carcinoma, ovarian cancer, prostate cancer, breast cancer, head and neck cancer, pancreatic cancer, mesothelioma, and/or a cancer of epithelial origin. In some embodiments, the disease is cervical cancer, head and neck squamous cell carcinoma, endometrial cancer, esophageal cancer, non-small cell lung cancer, or prostate cancer. In some embodiments, the disease or condition is a solid tumor cancer. In particular embodiments, the disease is colorectal cancer. In some embodiments, the disease is gastric cancer. In some embodiments, the disease is ovarian cancer. In some embodiments, the disease is breast cancer. In some embodiments, the disease is lung cancer. In some embodiments, the disease is non-small cell lung cancer. In some embodiments, the disease is head and neck cancer. In some embodiments, the disease is head and neck squamous cell carcinoma. In some embodiments, the disease is cervical cancer. In some embodiments, the disease is endometrial cancer. In some embodiments, the disease is prostate cancer. In some embodiments, the disease is esophageal cancer. In some embodiments, the disease is colon cancer. In some embodiments, the disease is pancreatic cancer. In some embodiments, the disease is mesothelioma.

### 16. Diagnostic Applications

In some embodiments, the antibodies or antibody conjugates provided herein are used in diagnostic applications. For example, an anti-tissue factor antibody or antibody conjugate may be useful in assays for tissue factor protein. In some aspects the antibody can be used to detect the expression of tissue factor in various cells and tissues. These assays may be useful, for example, in making a diagnosis and/or prognosis for a disease, such as a cancer.

In some aspects, a method of diagnosing a disease or condition in a subject in need thereof can comprise administering to the subject an effective amount of an isolated antibody, antibody conjugate or pharmaceutical composition thereof of the present disclosure.

In some diagnostic and prognostic applications, the antibody or antibody conjugate may be labeled with a detectable moiety. Suitable detectable moieties include, but are not limited to radioisotopes, fluorescent labels, and enzyme-substrate labels. In another embodiment, the anti-tissue factor antibody or antibody conjugate need not be labeled, and the presence of the antibody can be detected using a labeled antibody which specifically binds to the anti-tissue factor antibody.

### 17. Affinity Purification Reagents

The antibodies or antibody conjugates provided herein of the invention may be used as affinity purification agents. In this process, the antibodies or antibody conjugates may be immobilized on a solid phase such a resin or filter paper, using methods well known in the art. The immobilized antibody is contacted with a sample containing the tissue factor protein (or fragment thereof) to be purified, and thereafter the support is washed with a suitable solvent that will remove substantially all the material in the sample except the tissue factor protein, which is bound to the immobilized antibody. Finally, the support is washed with another suitable solvent, such as glycine buffer, pH 5.0 that will release the tissue factor protein from the antibody.

### 18. Kits

In some embodiments, an anti-tissue factor antibody or antibody conjugate provided herein is provided in the form of a kit, i.e., a packaged combination of reagents in predetermined amounts with instructions for performing a procedure. In some embodiments, the procedure is a diagnostic assay. In other embodiments, the procedure is a therapeutic procedure.

In some embodiments, the kit further comprises a solvent for the reconstitution of the anti-tissue factor antibody or antibody conjugate. In some embodiments, the anti-tissue factor antibody is provided in the form of a pharmaceutical composition.

### EXAMPLES

### Example 1: Generation and Primary Screening of Anti-TF Antibodies

Antibody Fab and scFv libraries were constructed using a standard overlap extension PCR protocol with mutagenic primers targeting complementary determining regions (CDRs). See Heckman and Pease, Nat. Protoc., 2007, 2:924-932; Stafford et al., 2014, Protein Eng. Des. Sel. 27:97-109, both incorporated by reference in their entireties. After multiple rounds of selection, the DNA from RT-PCR output was cloned into an optimized vector for cell-free expression using standard molecular biology techniques. All constructs were HIS- and FLAG-tagged to streamline purification and testing during screening.

Selections for novel antibodies were performed using standard ribosome display protocols. See Dreier and Pluckthun, Methods Mol. Biol., 2003, 687:283-306, Clifton, NJ; Hanes and Plückthun, Proc. Natl. Acad. Sci. U.S.A., 1997, 94:4937-4942, both incorporated by reference in their entireties. Fab and scFv ribosome display selections were performed according to published protocols. See Stafford et al., 2014, Protein Eng. Des. Sel. 27:97-109; Hanes and Plückthun, Proc. Natl. Acad. Sci. U.S.A., 1997, 94:4937-4942; both incorporated by reference in their entireties. After multiple rounds of selection, the DNA from RT-PCR output was cloned into an optimized vector for cell-free expression using standard molecular biology techniques. See Yin et al., mAbs, 2012, 4:217-225, incorporated by reference in its entirety. All constructs were HIS- and FLAG-tagged to streamline purification and testing during screening.

Antibody Fab libraries were constructed using a trastuzumab Fab sequence codon optimized for XpressCF® in a modified, commercially available p3 phagemid vector (Antibody Design Labs). Briefly, the phagemid vector was modified to express Fab heavy chains as a C-terminal p3 fusion proteins and regulatory regions (start codons, restriction enzyme sites, periplasmic leader sequences) were optimized for Fab display levels. Libraries were constructed using standard overlap expression PCR protocols with mutagenic primers targeting heavy chain complementary determining regions (CDRs) and rescued through electroporation in M13-K07 infected SS320 E. coli cells. See, Heckman and Pease, Nat. Protoc., 2007, 2:924-932; Rajan, S. & Sidhu, S. S., Methods Enzymol., 2012, 502:3-23, both incorporated by reference in their entireties. Immune libraries were similarly constructed using standard overlap PCR protocols, with heavy and light variable genes amplified from total RNA extracted from splenocytes. See Barbas II, CF & Burton, DR, Phage Display: A Laboratory Manual (2001), Cold Spring Harbor Laboratory Press, incorporated by reference in its entirety. Library selections were performed using standard phage display protocols. See Marks, J. D. & Bradbury, A., Methods Mol. Biol., 2004, 248:161-76; Rajan, S. & Sidhu, S. S., Methods Enzymol., 2012, 502:3-23; Barbas II, CF & Burton, DR, Phage Display: A Laboratory Manual (2001), Cold Spring Harbor Laboratory Press, each of which is incorporated herein by reference in its entirety. Following multiple selection rounds, Fab heavy chain pools were transferred into cell-free expression vectors for expression as His6 and FLAG-tagged IgG1. Immune scFvs were transferred into cell-free expression vectors with a 4 residue SKNK peptide leader found to facilitate expression. Immune scFvs were expressed as scFv-Fc with Flag and His6 C-terminal tags.

Libraries of antibody variants generated by selection workflow were transformed into E. coli and grown on agar plates with antibiotic (kanamycin). Individual colonies were grown in liquid broth (TB+kanamycin), and used as a template for DNA amplification via rolling circle amplification (RCA). The variants were then expressed in cell-free protein synthesis reactions as described in Zawada et al., Biotechnol. Bioeng., 2011, 108:1570-1578 and Cai, et al., Biotechnol. Prg., 2015, 823-831, each of which is incorporated by reference in its entirety.

Briefly, cell-free extracts were treated with 50 μM iodoacetamide for 30 min at room temperature (20° C.) and added to a premix containing cell-free components (see Groff et al., mAbs, 2014, 6:671-678 and Cai et al., Biotechnol. Prg., 2015, 823-831, each of which is incorporated by reference in its entirety) and 10% (v/v) RCA DNA template (approximately 10 μg/mL DNA) for HC variants of interest, in addition to 6 μg/mL Trastuzumab LC which is present for antibody assembly but is not varied in the library. Trastuzumab LC DNA was not added to CF reactions for scFvFc libraries. Sixty microliters of cell-free reactions were incubated at 30° C. for 12 hr on a shaker at 650 rpm in 96-well plates. Four hundred to one-thousand-two-hundred colonies were screened, depending on the predicted diversity of different selection campaigns.

Following synthesis, each reaction was diluted 1:50 into PBS (pH 7.4) with 0.1% Tween-20 and 0.2% bovine serum albumin. Diluted CF material was tested for TF binding via ELISA. Briefly, 1 μg/mL of human TF (Lake Pharma) or cynomolgus TF (Sino Bio #90885-C08H) or Neutravidin (NA) (ThermoFisher #31050) were coated to 384-well polystyrene plates overnight at 4° C. in 0.1M bicarbonate (pH 8.9) buffer before they were washed and blocked with 2% BSA in PBST (PBS, 0.1% Tween-20) for 1 hour at room temperature. The diluted CF material was applied to the ELISA plates for 1 hour at 30° C. Antibody binding was detected via HRP-conjugated anti-Flag antibody (Sigma) and detection with SuperSignal ELISA Pico Chemiluminescent Substrate (ThermoFisher #37069). Binding was reported as a ratio of TF ELISA signal over NA ELISA signal. The resulting antibodies are reported in Table 6, below.

The top leads from ELISA-based screening were cultured, and plasmid minipreps were performed using a QIAprep® 96 Turbo miniprep kit (Qiagen) according to the manufacturer's instructions. 7.5 μg/mL miniprepped HC DNA and 2.5 μg/mL of the trastuzumab LC was added to 4 mL cell-free reactions and incubated overnight for 12 hr at 30° C., at 650 rpm. In the case of IgG variants with a common Trastuzumab LC, 7.5 μg/mL of the HC variant DNA and 2.5 μg/mL of the common Trastuzumab LC were added to the reaction.

Antibodies from programs 2901 and 2842 did not have the common trastuzumab LC.

Expressed variants from clarified cell-free reactions were purified via Protein A purification using a semi-automated high throughput batch purification method. Briefly, purifications were performed in a 96-well plate format where 100 μL/well of Protein-A-Sepharose resin slurry (Invitrogen #101090) was equilibrated in phosphate buffered saline (PBS), incubated with 1 mL cell-free reaction for 30 minutes with mixing followed by two washes in PBS. Antibody variants were then eluted using 180 μL/well 0.1M glycine pH 2.0-2.5 (Santa Cruz Biotechnology), neutralized with 20 μL/well 1M Tris HCl pH 8.1, and buffer exchanged into PBS+10% sucrose using a 96-well Zeba plate (7 kD MWCO, ThermoFisher #89808). Purified antibodies were quantified via high throughput capillary electrophoresis using the LabChip GXII (Perkin Elmer) against a Herceptin standard curve, according to the manufacturer's instructions.

TABLE 6
Affinity Matured (2799) Antibodies

| | | SEQ ID | | SEQ ID |
|---|---|---|---|---|
| Antibody | V_{H} | NO. | V_{L} | NO. |
| SRP2799-A05 | SRP2799-A05 | 2727 | Trastuzumab | 3061 |
| (parent) | (parent) | | LC | |
| SRP2799-B03 | SRP2799-B03 | 2736 | Trastuzumab | 3061 |
| (parent) | (parent) | | LC | |
| SRP2799-B06 | SRP2799-B06 | 2739 | Trastuzumab | 3061 |
| (parent) | (parent) | | LC | |

Certain antibodies were generated by phage display selection in scFv format. The scFv candidates were reformatted to scFvFc format after selection and screened as scFvFcs. Selection and screening were carried out as described above.

Rabbit antibody leads SRP-2842-G04 and SRP-2842-B01 were selected for humanization.

Human germline framework sequences from preferred gene families (e.g., IGHV3, IGHV4 and IGHJ4) were used for “CDR-grafting” the heavy chain variable region of the selected rabbit lead antibodies. Some mutations were designed in the frameworks of “CDR-grafted” heavy chain variable region sequences. Similarly, preferred human germline framework sequences from kappa light chain families (e.g., IGKV1, IGKV3, IGKJ1 and IGKJ4) were used for “CDR-grafting” the light chain variable region of the selected rabbit lead antibodies. Some mutations were designed in the frameworks of “CDR-grafted” light chain variable region sequences.

Humanized heavy chain variable region and light chain variable region sequences were fused to human IGHG1 and IGKC constant region sequences respectively to construct full-length humanized antibody heavy chain and light chain sequences.

Rabbit scFv sequences correspond to clones with IDs starting with 2842 and humanized sequences are from clone IDs 2901.

Clones with IDs starting with 2900 are HC from phage display with common trastuzumab LC.

### Example 2: Preparation of SCFVS

A single-chain antibody is made in either the V_{H}V_{L} or V_{L}V_{H} orientation with a linker sequence between the V_{H} and V_{L} domains. Typically, scFv linkers are composed of (GGGGS)ₙ repeats where n=3, 4, 5, or 6 for linkers of 15, 20, 25, or 30 residues respectively, such as (GGGGS)₃ (SEQ ID NO: 3070). For cell-free expression, an N-terminal Met is added, but for mammalian expression a leader peptide is added. On the C-terminal end of the scFv, an Fc sequence can be added to extend in vivo half-life or the scFv can be used directly. An optional linker sequence can be incorporated between the scFv and the Fc. An exemplary scFv-Fc linker sequence is AAGSDQEPKSS (SEQ ID NO: 3071). C-terminal affinity tags can optionally be added to facilitate purification and assay development. An exemplary affinity tag is a C-terminal FlagHis tag GSGDYKDDDDKGSGHHHHHH (SEQ ID NO: 3069). A stop codon is typically inserted at the end of the sequence. Exemplary scFv's of the present disclosure include SEQ ID NOs: 3073 and 3079, with an N-terminal Met residue, a SKNK peptide leader, a V_{H} domain, a GGGGSGGGGSGGGGS (SEQ ID NO: 3070) linker, and a V_{L} domain. Exemplary scFvFc's of the present disclosure include these scFv's further including an AAGSDQEPKSS (SEQ ID NO: 3071) linker, an Fc domain, a FlagHis tag, and a stop codon.

Exemplary scFv's embodiment are illustrated in SEQ ID NOs: 3073 and 3079.

### Example 3: Melting Temperature, Affinity, and Kinetic Binding Analyses

A protein thermal shift assay was carried out by mixing the protein to be assayed with an environmentally sensitive dye (SYPRO Orange, Life Technologies #S-6650) and monitoring the fluorescence of the mixture in real time as it underwent controlled thermal denaturation. Protein solutions between 0.2-2 mg/mL were mixed at a 1:1 volumetric ratio with a solution of SYPRO Orange solution diluted 250-fold in PBS+10% sucrose. 10 μL aliquots of the protein-dye mixture were dispensed in quadruplicate in a 384-well microplate (Bio-Rad #MSP-3852). The plate was sealed with an optically clear sealing film (Bio-Rad #MSB-1001) and placed in a 384-well plate real-time thermocycler (Bio-Rad CFX384 Real Time System). The protein-dye mixture was heated from 25° C. to 95° C., at increments of 0.2° C. per cycle, allowing 3 seconds of equilibration at each temperature before taking a fluorescence measurement. At the end of the experiment, the transition melting temperatures (TM1 and TM2) were determined using the Bio-Rad CFX manager software. TM1 represents the melting temperature of the Fc domain. TM2 represents the melting temperature of the Fab domain. For scFvFc variants, typically only a single melting temperature was observed. Results are shown in Tables 7-11 below.

Anti-Fc polyclonal antibodies were immobilized onto a CM4 chip (Cytiva #BR100534) using amine coupling chemistry (Human Antibody Capture Kit, Cytiva #BR100839). The immobilization steps were carried out at a flow rate of 25 μL/min in 1×HBS-EP+buffer (Cytiva #BR100669). The sensor surfaces were activated for 7 min with a mixture of N-hydroxysuccinimide (NHS, 0.05 M) and 1-ethyl-3-(3-dimethylaminopropyl)-carbodiimide (EDC, 0.2 M). The anti-Fc polyclonal antibodies were injected over all 4 flow cells at a concentration of 25 μg/mL in 10 mM sodium acetate, pH 4.5, for 7 min. Ethanolamine (1 M, pH 8.5) was injected for 7 min to block any remaining activated groups. Approximately 6,000 response units (RU) of capture antibody was immobilized on each flow cell.

Off-rate and kinetic binding experiments were performed at 25° C. using 1×HBS-EP+buffer. Antibodies were injected over the anti-Fc surface at a concentration of 3 μg/mL for 12 seconds at a flow rate of 10 μL/min on flow cells 2, 3 and 4, followed by a buffer wash for 30 seconds at the same flow rate. Single point off-rate analysis was carried out using an antigen concentration of 100 nM. Detailed kinetic characterization of antibody samples was carried out with a range of antigen concentrations from about 1 to 100 nM and 1 injection of 0 nM antigen (for example, 100, 25, 12.5, 3.125, 0.781 and 0 nM). After capturing ligand (antibody) on the anti-Fc surface, the analyte (human TF, Lake Pharma; cynomolgus TF, Sino Bio #90885-C08H; mouse TF, R&D Systems #3178-PA) was bound for 180 seconds, followed by a 420 second dissociation phase at a flow rate of 50 L/min. Between each ligand capture and analyte binding cycle, regeneration was carried out using 2 injections of 10 mM glycine pH 2.0 for 30 seconds at 30 μL/min, followed by a 30 second buffer wash step.

The data were fit with the Biacore T200 Evaluation software, using a 1:1 Langmuir binding model. K_{D} (affinity, nM) was determined as a ratio of the kinetic rate constants calculated from the fits of the association and dissociation phases.

### Example 4: Flow Cytometry-Based Cell Binding Assay

HEK293T, BxPC3 cells (TF positive) and NCI-H1703 (TF negative) cells were obtained from ATCC (American Type Culture Collection) and maintained in DMEM: F12 (1:1), high glucose (Corning) supplemented with 10% heat-inactivated fetal bovine serum (ThermoScientific), 1× Penicillin/Streptomycin (Corning) and 2 mmol glutamax (Thermo Fisher Scientific). To generate cells that stably expressing human tissue factor (HEK293T-humanTF) or cynomolgus tissue factor (HEK293T-cynoTF), HEK293T cells were transfected with plasmid vectors that contain the corresponding protein sequence and stable clones were selected with puromycin.

For a fluorescence-activated cell sorting (FACS) cell-binding assay, cells were collected by Accutase and counted by the Vi-CELL Cell Viability Analyzersharvested and, then re-suspended in FACS buffer (DPBS buffer supplemented with 1% bovine serum albumin). A total of 200,000 cells in 100 μL were incubated on ice with 100 μL of anti-TF antibodies in each well of a 96-well plate. Cells were washed twice with ice-cold FACS buffer and then incubated on ice for 60 mins with 5 μg/mL Alexa 647-labeled donkey anti-human Fc antibody (Jackson ImmunoResearch). Unstained cells and cells stained with secondary antibody alone were used as controls. Cells were washed twice with FACS buffer and analyzed using a BD FACS Canto system. Geometric mean fluorescence intensities (MFI) were fitted using non-linear regression analysis with one site specific binding equation on GraphPad Prism (GraphPad Software; La Jolla, CA).

### Example 5: Thrombin Generation Assay

Antibodies and antibody drug conjugates (ADC) as described below were assessed for inhibition of thrombin production using a Thrombin Generation Assay Kit (Technoclone #5006010). All kit reagents were prepared as described in the manufacturer's protocol. A thrombin calibration curve was generated using the kit's thrombin standard and thrombin generation in test samples was calculated using this standard curve. Antibodies were tested at 100 nM and dilutions were made using the kit's assay buffer. In each well of a black Maxisorp plate (NUNC #475515), 10 μL of the kit's RC reagent was incubated with 5 μL of the antibody dilution for 10 minutes at room temperature. 35 μL of the kit's human plasma was added to each well along with 50 μL of the thrombin fluorogenic substrate. The fluorescence at 360 nm/360 nm was read immediately, every minute for 60 minutes at 37° C. Relative fluorescence units were converted to thrombin concentration based on the calibration curve.

### Example 6: Cell-Killing Analysis

The internalization ability of the lead antibodies was evaluated by an ADC cell killing assay on Tissue Factor positive BxPC3 cells. A total of 625 cells in a volume of 25 μL were seeded in each well of a 384-well flat bottom white polystyrene plate. Lead antibodies were formulated at 4-fold starting concentration in the cell culture medium and filtered through MultiScreenHTS 96-Well Filter Plates (Millipore; Billerica, Massachusetts). 12.5 μL of antibody was added into each treatment well, and 12.5 μL of anti-human Fc nanobody conjugated to SC236 was then added into each well at a fixed final concentration of 20 nM. Assay plates were cultured at 37° C. in a CO₂ incubator for 120 hrs before assay. For cell viability measurement, 30 μL of Cell Titer-Glo® reagent (Promega Corp. Madison, WI) was added into each well, and plates were processed as per product instructions. Relative luminescence was measured on an ENVISION® plate reader (Perkin-Elmer; Waltham, MA). Relative luminescence readings were converted to percent viability using untreated cells as controls. Data was fitted with non-linear regression analysis, using a log (inhibitor) vs. response-variable slope, 4 parameter fit with GraphPad Prism (GraphPad v 5.0, Software; San Diego, California).

### Example 7: FX Conversion Assay

Antibodies were assessed for inhibition of FX activation using a Tissue Factor Activity Assay Kit (Abcam #ab 108906). The kit's reagents were prepared as described in the manufacturer's protocol. Antibodies were tested at 10 nM, 100 nM, and 1000 nM. All dilutions were made in the sample dilution buffer provided in the kit. 10 μL of the tissue factor reagent was incubated with 10 μL of antibody at room temperature for 20 minutes. 10 μL each of the FVII reagent and the FX reagent were diluted with 40 μL of the kit's assay diluent and added to each well of the provided 96-well microplate. The pre-incubated TF+antibody was added to the FVII+FX micture and incubated at 37° C. for 30 minutes. 20 μL of FXa substrate was added to each well. The absorbance at OD₄₀₅ was read immediately, every 5 minutes or 25 minutes while incubating at 37° C. The percent inhibition of FX conversion for 1000 nM antibody at the final timepoint was calculated relative to a no-antibody control.

### Example 8: Characteristics of Illustrative Anti-TF Antibodies

Tables 7 through 20 show results obtained using the illustrative antibodies described herein.

Tables 7 through 8 show results for melting temperatures from affinity-matured antibodies from initial primary screen leads, from mouse immune library scFvFc candidates from the initial primary screen and for humanized rabbit antibodies.

Tables 9 through 13 show results obtained for single point off-rates for affinity-matured antibodies form initial primary screen leads, for scFvFc candidates for humanization as well as melting temperatures for rabbit immune library scFvFc candidates for humanization, for humanized rabbit antibodies, for affinity-matured antibodies and humanized rabbit antibodies, for mouse scFvFc variants, and for lead antibody/ADC candidates.

Tables 14 through 18 show results for cell binding and/or cell killing for illustrative antibodies of the disclosure.

Tables 19 through 20 show results for FX inhibition assays for illustrative antibodies of the disclosure.

TABLE 7
Melting Temperatures for Affinity-Matured Antibodies
from Initial Primary Screen Leads

| | DSF |
|---|---|
| Variant ID | TM2 (° C.) |
| SRP2799-A05 (parent) | 81.4 |
| SRP2799-B03 (parent) | 82.1 |
| SRP2799-B06 (parent) | 85.7 |
| SRP2900-A01 | 78.1 |
| SRP2900-A02 | 83.6 |
| SRP2900-A03 | 77.2 |
| SRP2900-A04 | 78.8 |
| SRP2900-A06 | 81.5 |
| SRP2900-A07 | 81.9 |
| SRP2900-A08 | 80.3 |
| SRP2900-A09 | 82.2 |
| SRP2900-A10 | 79.4 |
| SRP2900-A11 | 82.3 |
| SRP2900-B01 | 79.8 |
| SRP2900-B02 | 80.3 |
| SRP2900-B03 | 81.6 |
| SRP2900-B04 | 82.5 |
| SRP2900-B05 | 81.9 |
| SRP2900-B06 | 80.3 |
| SRP2900-B07 | 85.5 |
| SRP2900-B08 | 75.4 |
| SRP2900-B09 | 82.8 |
| SRP2900-B10 | 76.2 |
| SRP2900-B11 | 78.2 |
| SRP2900-C01 | 82.1 |
| SRP2900-C02 | 80.4 |
| SRP2900-C03 | 80.8 |
| SRP2900-C04 | 81.3 |
| SRP2900-C05 | 82.7 |
| SRP2900-C06 | 80.7 |
| SRP2900-C07 | 79.3 |
| SRP2900-C09 | 81.6 |
| SRP2900-C10 | 81.7 |
| SRP2900-C11 | 82.4 |
| SRP2900-D01 | 84.2 |
| SRP2900-D02 | 82 |
| SRP2900-D03 | 81.4 |
| SRP2900-D04 | 79.3 |
| SRP2900-D05 | 84 |
| SRP2900-D06 | 81 |
| SRP2900-D07 | 84.6 |
| SRP2900-D08 | 83.7 |
| SRP2900-D09 | 82.5 |
| SRP2900-D10 | 78.7 |
| SRP2900-D11 | 78.6 |
| SRP2900-E01 | 82.7 |
| SRP2900-E02 | 83.4 |
| SRP2900-E03 | 84.1 |
| SRP2900-E04 | 76.8 |
| SRP2900-E05 | 83 |
| SRP2900-E06 | 81.7 |
| SRP2900-E07 | 84.9 |
| SRP2900-E08 | 82 |
| SRP2900-E09 | 79.5 |
| SRP2900-E10 | 81.9 |
| SRP2900-E11 | 80.3 |
| SRP2900-F02 | 81.8 |
| SRP2900-F03 | 76.3 |
| SRP2900-F04 | 81.7 |
| SRP2900-F05 | 83.6 |
| SRP2900-F06 | 79.7 |
| SRP2900-F07 | 83.1 |
| SRP2900-F08 | 83.2 |
| SRP2900-F09 | 83.5 |
| SRP2900-F10 | 82.1 |
| SRP2900-F11 | 82.3 |
| SRP2900-G01 | 80.8 |
| SRP2900-G02 | 81.4 |
| SRP2900-G03 | 82.5 |
| SRP2900-G04 | 79.3 |
| SRP2900-G05 | 81.3 |
| SRP2900-G06 | 83.1 |
| SRP2900-G07 | 83 |
| SRP2900-G08 | 78.3 |
| SRP2900-G09 | 82.5 |
| SRP2900-G10 | 79 |
| SRP2900-G11 | 80.6 |
| SRP2900-H01 | 79.5 |
| SRP2900-H02 | 80.4 |
| SRP2900-H03 | 81.6 |
| SRP2900-H04 | 80.7 |
| SRP2900-H05 | 81.8 |
| SRP2900-H06 | 82 |
| SRP2900-H07 | 83.3 |
| SRP2900-H08 | 79.8 |
| SRP2900-H09 | 83.9 |
| SRP2900-H10 | 78.8 |
| SRP2900-H11 | 80.4 |

TABLE 8
Melting Temperatures for Humanized Rabbit Antibodies.

| | DSF |
|---|---|
| Variant ID | TM2 (° C.) |
| 2842-B01 HC1/LC1 | 87.3 |
| 2842-B01 HC1/LC2 | 82.4 |
| 2842-B01 HC1/LC3 | 87.6 |
| 2842-B01 HC2/LC1 | 86.3 |
| 2842-B01 HC2/LC3 | 87.6 |
| 2842-B01 HC3/LC1 | 86.5 |
| 2842-B01 HC3/LC2 | 81.5 |
| 2842-G04 HC1/LC2 | 80 |
| 2842-G04 HC2/LC1 | 80.4 |
| 2842-G04 HC2/LC2 | 80.2 |
| 2842-G04 HC3/LC2 | 86.7 |
| 2842-G04 HC3/LC3 | 82.3 |

TABLE 9
Single point off-rates for affinity-matured
antibodies from initial primary screen leads.

| | Biacore human | Biacore |
|---|---|---|
| | TF single point | cynomolgus TF |
| | off-rate | single point |
| | kinetics | off-rate kinetics |
| Variant ID | kd (1/s) | kd (1/s) |
| SRP2799-A05 | 3.04E−03 | 3.37E−02 |
| (parent) | | |
| SRP2799-B03 | 4.15E−03 | 1.69E−03 |
| (parent) | | |
| SRP2799-B06 | 8.27E−03 | 1.66E−03 |
| (parent) | | |
| SRP2900-A02 | 1.16E−03 | 7.61E−04 |
| SRP2900-C01 | 1.83E−03 | 1.18E−03 |
| SRP2900-C11 | 8.42E−04 | 2.57E−03 |
| SRP2900-E02 | 5.28E−04 | 1.82E−03 |
| SRP2900-E06 | 9.78E−04 | 6.89E−04 |
| SRP2900-E07 | 5.11E−04 | 1.13E−03 |
| SRP2900-F02 | 2.74E−03 | 6.96E−04 |
| SRP2900-F06 | 4.23E−03 | 1.84E−03 |
| SRP2900-F08 | 1.31E−03 | 6.65E−04 |
| SRP2900-F09 | 1.39E−03 | 2.15E−03 |
| SRP2900-F11 | 2.04E−03 | 1.15E−03 |
| SRP2900-G02 | 4.15E−03 | 1.41E−03 |
| SRP2900-G03 | 6.99E−04 | 1.95E−03 |
| SRP2900-G05 | 1.15E−03 | 6.67E−04 |
| SRP2900-H06 | 3.47E−03 | 6.48E−04 |
| SRP2900-H07 | 2.05E−03 | 1.30E−03 |
| SRP2900-H09 | 1.69E−03 | 5.84E−03 |
| SRP2900-H11 | 2.50E−03 | 9.46E−04 |

TABLE 10
Rabbit immune library scFvFc candidates for humanization.

| | Biacore | Biacore | |
|---|---|---|---|
| | human TF | cynomolgus TF | |
| | single point | single point | |
| | off-rate | off-rate | |
| | kinetics | kinetics | DSF |
| Variant ID | kd (1/s) | kd (1/s) | TM (° C.) |
| 2842-B01 | 3.03E−04 | 1.27E−03 | 58.3 |
| (scFvFc parent) | | | |
| 2842-G04 | 6.04E−04 | 1.41E−03 | 54.3 |
| (scFvFc parent) | | | |

TABLE 11
Single point off-rates for humanized rabbit antibodies.

| | Biacore | Biacore |
|---|---|---|
| | human TF | cynomolgus TF |
| | single point | single point |
| | off-rate | off-rate |
| | kinetics | kinetics |
| Variant ID | kd (1/s) | kd (1/s) |
| 2842-B01 HC1/LC1 | 7.27E−04 | 3.52E−03 |
| 2842-B01 HC1/LC2 | 6.91E−04 | 1.42E−03 |
| 2842-B01 HC1/LC3 | 5.40E−04 | 1.17E−03 |
| 2842-B01 HC2/LC1 | 5.23E−04 | 9.91E−04 |
| 2842-B01 HC2/LC3 | 7.26E−04 | 3.14E−03 |
| 2842-B01 HC3/LC1 | 4.16E−04 | 1.87E−03 |
| 2842-B01 HC3/LC2 | 3.61E−04 | 7.82E−04 |
| 2842-G04 HC2/LC1 | 6.78E−04 | 3.03E−03 |
| 2842-G04 HC2/LC2 | 7.36E−04 | 1.70E−03 |

TABLE 12
Biacore kinetics for affinity-matured antibodies and humanized rabbit antibodies.

| | | Biacore human TF kinetics | | | Biacore cynomolgus TF kinetics | | |
|---|---|---|---|---|---|---|---|
| | | ka | | | ka | | |
| mAb # | Variant ID | (1/Ms) | kd (1/s) | KD (M) | (1/Ms) | kd (1/s) | KD (M) |
| 36 | 2900-A02 | 3.03E+05 | 1.44E−03 | 4.77E−09 | 2.85E+05 | 7.43E−04 | 2.61E−09 |
| 42 | 2900-C11 | 1.88E+05 | 6.74E−04 | 3.59E−09 | 1.28E+05 | 3.09E−03 | 2.42E−08 |
| 43 | 2900-F06 | 3.11E+05 | 5.94E−03 | 1.91E−08 | 3.16E+05 | 2.31E−03 | 7.33E−09 |
| 44 | 2900-F09 | 3.62E+05 | 1.96E−03 | 5.42E−09 | 1.64E+05 | 3.09E−03 | 1.89E−08 |
| 45 | 2900-H06 | 5.47E+05 | 4.26E−03 | 7.78E−09 | 1.21E+06 | 3.09E−04 | 2.55E−10 |
| 46 | 2900-H09 | 3.62E+05 | 2.26E−03 | 6.24E−09 | 1.64E+05 | 6.81E−03 | 4.16E−08 |
| 37 | 2842-B01 | 5.72E+04 | 4.18E−04 | 7.30E−09 | 7.24E+04 | 1.13E−03 | 1.56E−08 |
| | HC3/LC2 | | | | | | |
| 47 | 2842-B01 | 5.40E+04 | 4.22E−04 | 7.81E−09 | 8.27E+04 | 1.66E−03 | 2.01E−08 |
| | HC3/LC1 | | | | | | |
| 48 | 2842-B01 | 7.12E+04 | 1.24E−03 | 1.74E−08 | 1.21E+05 | 7.35E−03 | 6.07E−08 |
| | HC1/LC3 | | | | | | |
| 49 | 2842-G04 | 6.25E+04 | 8.65E−04 | 1.38E−08 | 8.08E+04 | 2.41E−03 | 2.99E−08 |
| | HC2/LC2 | | | | | | |

TABLE 13
Biacore kinetics for lead antibody/ADC.

| | | | | | Biacore cynomolgus TF | | |
|---|---|---|---|---|---|---|---|
| | | | | | kinetics | | |
| Conjugate | | Biacore human TF kinetics | | | ka | kd | |
| or mAb # | Description | ka (1/Ms) | kd (1/s) | KD (M) | (1/Ms) | (1/s) | KD (M) |
| mAb #41 | 2900-A02 | 4.87E+05 | 1.82E−03 | 3.73E−09 | 7.52E+05 | 8.43E−04 | 1.12E−09 |
| | F241/F404TAG | | | | | | |
| Conjugate | 2900-A02 | 5.08E+05 | 1.89E−03 | 3.71E−09 | 7.47E+05 | 8.92E−04 | 1.19E−09 |
| 2 | F241/F404 LP1 | | | | | | |

TABLE 14
Cell Killing and Cell Binding Activity
of Antibodies from SRP2799.

| | | | BxPC3 Cell | | BxPC3 Cell | |
|---|---|---|---|---|---|---|
| | | | Killing | | Binding | |
| | Expression, | % | EC50 | Span | Kd | Bmax |
| Sample | IgG (ug/mL) | monomer | (nM) | (%) | (nM) | (MFI) |
| SRP2799-A05 | 2622 | 89 | 0.092 | 95 | 26.1 | 52703 |
| SRP2799-B03 | 1758 | 81 | 0.072 | 97 | 4.7 | 45417 |
| SRP2799-B06 | 1453 | 78 | 0.06 | 96 | 4.1 | 46326 |

TABLE 15
Cell Killing Activity of Antibodies from SRP2842.

| | |
|---|---|
| [scFv-Fc] | BxPC3 Cell Killing |

| Sample | ug/mL | EC50 (nM) | Span (%) |
|---|---|---|---|
| SRP2842-B01 | 761 | 0.057 | 91.1 |
| SRP2842-G04 | 998 | 0.059 | 94.2 |
| Tisotumab | 4100 | 0.023 | 94.7 |

TABLE 16
Cell Binding Activity of Antibodies from SRP2900 and SRP2842.

| | | HEK293T-human | | | | | |
|---|---|---|---|---|---|---|---|
| | | TF | | HEK293T-cyno TF | | HEK293T | |
| | | Bmax | Kd | Bmax | Kd | Bmax | Kd |
| mAb # | Sample Name | (MFI) | (nM) | (MFI) | (nM) | (MFI) | (nM) |
| 36 | 2900-A02 | 306977 | 1.2 | 443667 | 14 | 1503 | NC |
| 42 | 2900-C11 | 274969 | 0.8 | 426333 | 16 | 1525 | NC |
| 43 | 2900-F06 | 253290 | 1.7 | 419173 | 21 | 1401 | NC |
| 44 | 2900-F09 | 295588 | 0.9 | 428667 | 29 | 2046 | NC |
| 45 | 2900-H06 | 302074 | 1.3 | 441333 | 7.5 | 1732 | NC |
| 46 | 2900-H09 | 299871 | 0.93 | 482667 | 38 | 1253 | NC |
| 37 | 2842- | 334235 | 3.9 | 493000 | 3.9 | 1062 | NC |
| | B01_HC3/LC2 | | | | | | |
| 47 | 2842- | 351065 | 3.3 | 500667 | 3.2 | 1285 | NC |
| | B01_HC3/LC1 | | | | | | |
| 48 | 2842- | 317623 | 2.3 | 485667 | 3.4 | 1232 | NC |
| | B01_HC1/LC3 | | | | | | |
| 49 | 2842- | 243765 | 1.4 | 464667 | 5.1 | 1366 | NC |
| | G04_HC2/LC2 | | | | | | |

TABLE 17
Cell Killing and Cell Binding Activity
of Antibodies from SRP2901.

| | | BxPC3 Cell Killing | | BxPC3 Cell Binding | |
|---|---|---|---|---|---|
| | [IgG] | EC50 | Span | Kd | Bmax |
| Sample | ug/mL | (nM) | (%) | (nM) | (MFI) |
| SRP2901-B05 | 395 | 0.059 | 84 | 0.389 | 42810 |
| SRP2901-D03 | 456 | 0.184 | 77.4 | 0.344 | 29147 |
| SRP2901-E03 | 252 | 0.166 | 92.4 | 0.386 | 35248 |
| SRP2901-F01 | 593 | 0.364 | 87 | 0.61 | 25351 |
| SRP2842-B01 | 761 | 0.057 | 91.1 | 0.089 | 39002 |
| SRP2842-G04 | 998 | 0.059 | 94.2 | 0.058 | 34919 |

TABLE 18
Cell Killing Activity of Antibodies from SRP2799, 2900 and 2916.

| | |
|---|---|
| [IgG] | BxPC3 Cell Killing |

| Sample | ug/mL | EC50 (nM) | Span (%) |
|---|---|---|---|
| SRP2799-A05 | 1243 | 0.054 | 98.92 |
| SRP2799-B03 | 1088 | 0.059 | 96.26 |
| SRP2799-B06 | 1248 | 0.06 | 88.79 |
| SRP2900-A02 | 1286 | 0.08 | 84.59 |
| SRP2900-C11 | 775 | 0.057 | 85.14 |
| SRP2900-E07 | 787 | 0.174 | 81.95 |
| SRP2900-F02 | 1197 | 0.056 | 95.85 |
| SRP2900-F06 | 708 | 0.108 | 87.57 |
| SRP2900-F08 | 1157 | 0.057 | 93.67 |
| SRP2900-F09 | 1194 | 0.059 | 90.6 |
| SRP2900-F11 | 1293 | 0.069 | 90.07 |
| SRP2900-G02 | 1106 | 0.104 | 93.11 |
| SRP2900-G05 | 1179 | 0.059 | 95.94 |
| SRP2900-H06 | 1240 | 0.061 | 94.04 |
| SRP2900-H09 | 1199 | 0.035 | 88.18 |
| SRP2900-H11 | 991 | 0.067 | 90.92 |

TABLE 19
Inhibition of FX Conversion by Affinity-Matured
Antibodies from Initial Primary Screen Leads.

| | FX conversion |
|---|---|
| Variant ID | (% inhibition) |
| SRP2799-A05 (parent) | 1.12% |
| SRP2799-B03 (parent) | 2.41% |
| SRP2799-B06 (parent) | −1.01% |
| SRP2900-A01 | 36.97% |
| SRP2900-A02 | 0.21% |
| SRP2900-A03 | 26.28% |
| SRP2900-A04 | 38.58% |
| SRP2900-A06 | 36.84% |
| SRP2900-A07 | 28.44% |
| SRP2900-A08 | 41.86% |
| SRP2900-A09 | 46.87% |
| SRP2900-A11 | 31.74% |
| SRP2900-B01 | 38.70% |
| SRP2900-B02 | 26.97% |
| SRP2900-B03 | 44.14% |
| SRP2900-B04 | 46.27% |
| SRP2900-B05 | 35.51% |
| SRP2900-B06 | 28.16% |
| SRP2900-B07 | 42.25% |
| SRP2900-B08 | 43.33% |
| SRP2900-B09 | 35.02% |
| SRP2900-B10 | 33.33% |
| SRP2900-B11 | 22.38% |
| SRP2900-C01 | 16.43% |
| SRP2900-C02 | 29.56% |
| SRP2900-C04 | 36.70% |
| SRP2900-C05 | 35.67% |
| SRP2900-C07 | 41.57% |
| SRP2900-C10 | 42.07% |
| SRP2900-C11 | 12.44% |
| SRP2900-D01 | 32.68% |
| SRP2900-D02 | 32.11% |
| SRP2900-D03 | 37.37% |
| SRP2900-D04 | 45.95% |
| SRP2900-D05 | 33.58% |
| SRP2900-D06 | 46.18% |
| SRP2900-D07 | 47.42% |
| SRP2900-D08 | 44.87% |
| SRP2900-D09 | 30.02% |
| SRP2900-D10 | 36.95% |
| SRP2900-D11 | 51.41% |
| SRP2900-E01 | 40.44% |
| SRP2900-E02 | 17.72% |
| SRP2900-E03 | 48.68% |
| SRP2900-E04 | 25.91% |
| SRP2900-E05 | 23.50% |
| SRP2900-E06 | 18.71% |
| SRP2900-E07 | 15.61% |
| SRP2900-E08 | 45.79% |
| SRP2900-E09 | 32.16% |
| SRP2900-E10 | 50.72% |
| SRP2900-E11 | 39.94% |
| SRP2900-F02 | 0.23% |
| SRP2900-F04 | 47.26% |
| SRP2900-F05 | 25.11% |
| SRP2900-F06 | 1.72% |
| SRP2900-F07 | 30.27% |
| SRP2900-F08 | 9.78% |
| SRP2900-F09 | 5.23% |
| SRP2900-F10 | 32.52% |
| SRP2900-F11 | 6.38% |
| SRP2900-G01 | 25.06% |
| SRP2900-G02 | −0.11% |
| SRP2900-G03 | 20.01% |
| SRP2900-G04 | 31.24% |
| SRP2900-G05 | 12.05% |
| SRP2900-G06 | 21.37% |
| SRP2900-G07 | 38.31% |
| SRP2900-G08 | 38.93% |
| SRP2900-G09 | 30.43% |
| SRP2900-G10 | 48.66% |
| SRP2900-G11 | 40.85% |
| SRP2900-H01 | 30.48% |
| SRP2900-H02 | 40.42% |
| SRP2900-H03 | 40.81% |
| SRP2900-H04 | 36.26% |
| SRP2900-H05 | 36.24% |
| SRP2900-H06 | 4.04% |
| SRP2900-H07 | 13.54% |
| SRP2900-H08 | 32.22% |
| SRP2900-H09 | 4.48% |
| SRP2900-H10 | 45.40% |
| SRP2900-H11 | −3.63% |

TABLE 20
Inhibition of FX Conversion by scFvFc Parents
and Humanized Rabbit Antibodies.

| | FX conversion |
|---|---|
| Variant ID | (% inhibition) |
| 2842-B01 (scFvFc parent) | 2.54% |
| 2842-G04 (scFvFc parent) | 2.29% |
| 2842-B01 HC1/LC1 | −0.45% |
| 2842-B01 HC1/LC2 | −0.99% |
| 2842-B01 HC1/LC3 | 0.77% |
| 2842-B01 HC2/LC1 | −3.76% |
| 2842-B01 HC2/LC3 | −0.42% |
| 2842-B01 HC3/LC1 | −1.60% |
| 2842-B01 HC3/LC2 | −0.84% |
| 2842-G04 HC2/LC1 | −22.15% |
| 2842-G04 HC2/LC2 | 0.62% |

### SYNTHETIC EXAMPLES

The present examples below describe the preparation and testing of the following linker-payloads, antibodies and conjugates.

### Linker Payloads.

| | Linker payload | Linker release |
|---|---|---|
| LP No. | description | mechanism |
| 1 | DBCO-Valcit-pAB- | Cathepsin cleavable |
| | hemiasterlin | |
| 2 | DBCO-β-Glucuronide- | β-Glucuronidase cleavable |
| | PEG12-Exatecan | |
| 3 | DBCO-nnAA-PEG13- | Cathepsin cleavable |
| | VKG-Exatecan | |
| 4 | DBCO-nnAA-PEG13- | Legumain (LGMN) cleavable |
| | AAN-Exatecan | |
| 5 | DBCO-nnAA-PEG13- | Cathepsin cleavable |
| | AAA-Exatecan | |

### Antibodies.

| | | | LC and/or | |
|---|---|---|---|---|
| | | Conjugation | Conjugation | |
| mAb | Name | site on HC | site | mAb |
| 36 | aTF_2900- | Y180/F404 | (SEQID | 2900-A02 |
| | A02_HC_Y180/ | (SEQ ID | NO: 3179) | HC/trastuzumab |
| | F404TAG_SerOpt | NO:3168) | | LC |
| | pAzMeF; | | | |
| | Trastuzumab | | | |
| | LC SerOpt | | | |
| 37 | aTF_2842- | Y180/F404 | (SEQ ID | 2842-B01- |
| | B01_Hc3_Y180/ | (SEQ ID | NO: 3180) | Hc3/Lc2 |
| | F404TAG | NO: 3169) | | |
| | pAzMeF; | | | |
| | aTF_2842- | | | |
| | B01_Lc2 | | | |
| 38 | aTF_2900- | Y180/F404 | K42/E161 | 2900-A02 |
| | A02_HC_Y180/ | (SEQ ID | (SEQ ID | HC/trastuzumab |
| | F404TAG_SerOpt | NO: 3168) | NO: 3184) | LC |
| | pAzMeF; | | | |
| | trastuzumab | | | |
| | LC SerOpt | | | |
| | K42TAG/ | | | |
| | E161TAG/ | | | |
| | TCT162AGC | | | |
| | pAzMeF | | | |
| 39 | aTF_2900- | Y180/F404 | K42 | 2900-A02 |
| | A02_HC_Y180/ | (SEQ ID | (SEQ ID | HC/trastuzumab |
| | F404TAG_SerOpt | NO: 3168) | NO: 3185) | LC |
| | pAzMeF; | | | |
| | trastuzumab | | | |
| | LC SerOpt | | | |
| | K42TAG | | | |
| | pAzMeF | | | |
| 40 | aTF_2900- | Y180/F241/F404 | K42 | 2900-A02 |
| | A02_HC_Y180/F241/ | (SEQ ID | (SEQ ID | HC/trastuzumab |
| | F404TAG_SerOpt | NO: 3177) | NO: 3185) | LC |
| | pAzMeF; | | | |
| | trastuzumab | | | |
| | LC SerOpt | | | |
| | K42TAG | | | |
| | pAzMeF | | | |
| 41 | aTF_2900- | F241/F404 | (SEQ ID | 2900-A02 |
| | A02_HC_F241/ | (SEQ ID | NO: 3179) | HC/trastuzumab |
| | F404TAG_SerOpt | NO: 3178) | | LC |
| | pAzMeF; | | | |
| | Trastuzumab | | | |
| | LC SerOpt | | | |
| 42 | aTF_2900- | Y180/F404 | (SEQ ID | 2900-A02 |
| | C11_HC_Y180/ | (SEQ ID | NO: 3179) | HC/trastuzumab |
| | F404TAG_SerOpt; | NO: 3186) | | LC |
| | Trastuzumab | | | |
| | LC SerOpt | | | |
| 43 | aTF_2900- | Y180/F404 | (SEQ ID | 2900-F06 |
| | F06_HC_Y180/ | (SEQ ID | NO: 3179) | HC/trastuzumab |
| | F404TAG_SerOpt; | NO: 3187) | | LC |
| | Trastuzumab | | | |
| | LC SerOpt | | | |
| 44 | aTF_2900- | Y180/F404 | (SEQ ID | 2900-F09 |
| | F09_HC_Y180/ | (SEQ ID | NO: 3179) | HC/trastuzumab |
| | F404TAG_SerOpt; | NO: 3188) | | LC |
| | Trastuzumab | | | |
| | LC SerOpt | | | |
| 45 | aTF_2900- | Y180/F404 | (SEQ ID | 2900-H06 |
| | H06_HC_Y180/ | (SEQ ID | NO: 3179) | HC/trastuzumab |
| | F404TAG_SerOpt; | NO: 3189) | | LC |
| | Trastuzumab | | | |
| | LC SerOpt | | | |
| 46 | aTF_2900- | Y180/F404 | (SEQ ID | 2900-H09 |
| | H09_HC_Y180/ | (SEQ ID | NO: 3179) | HC/trastuzumab |
| | F404TAG_SerOpt; | NO: 3190) | | LC |
| | Trastuzumab | | | |
| | LC SerOpt | | | |
| 47 | aTF_2842- | Y180/F404 | (SEQ ID | |
| | B01_HC3_Y180/ | (SEQ ID | NO: 3181) | |
| | F404TAG; | NO: 3169) | | |
| | aTF_2842- | | | |
| | B01_LC1 | | | |
| 48 | aTF_2842- | Y180/F404 | (SEQ ID | |
| | B01_HC1_Y180/ | (SEQ ID | NO: 3182) | |
| | F404TAG; | NO: 3175) | | |
| | aTF_2842- | | | |
| | B01_LC3 | | | |
| 49 | aTF_2842- | Y180/F404 | (SEQ ID | |
| | G04_HC2_Y180/ | (SEQ ID | NO: 3183) | |
| | F404TAG; | NO: 3176) | | |
| | aTF_2842- | | | |
| | G04_LC2 | | | |
| 50 | | Y180/F241/ | (SEQ ID | |
| | | Y391/F404 | NO: 3179) | |
| | | (SEQ ID | | |
| | | NO: 3191) | | |

### Antibody-Drug Conjugates.

| | Conjugation | Conjugation | | |
|---|---|---|---|---|
| Conjugate | site on HC | site on LC | mAb | LP |
| 1 | Y180/F404 | (SEQ ID | 2900-A02 | LP1 |
| | (SEQ ID | NO: 3179) | | |
| | NO: 3168) | | | |
| 2 | F241/F404 | (SEQ ID | 2900-A02 | LP1 |
| | (SEQ ID | NO: 3179) | | |
| | NO: 3178) | | | |
| 3 | Y180/F404 | K42 | 2900-A02 | LP1 |
| | (SEQ ID | (SEQ ID | | |
| | NO: 3168) | NO: 3185) | | |
| 4 | Y180/F241/ | (SEQ ID | 2900-A02 | LP1 |
| | F404 | NO: 3179) | | |
| | (SEQ ID | | | |
| | NO: 3177) | | | |
| 5 | Y180/F404 | K42/E161 | 2900-A02 | LP1 |
| | (SEQ ID | (SEQ ID | | |
| | NO: 3168) | NO: 3184) | | |
| 6 | Y180/F241/ | K42 | 2900-A02 | LP1 |
| | F404 | (SEQ ID | | |
| | (SEQ ID | NO: 3185) | | |
| | NO: 3177) | | | |
| 7 | F241/F404 | (SEQ ID | 2900-A02 | LP2 |
| | (SEQ ID | NO: 3179) | | |
| | NO: 3178) | | | |
| 8 | Y180/F241/ | K42 | 2900-A02 | LP2 |
| | F404 | (SEQ ID | | |
| | (SEQ ID | NO: 3185) | | |
| | NO: 3177) | | | |
| 9 | Y180/F404 | (SEQ ID | 2900-A02 | LP2 |
| | (SEQ ID | NO: 3179) | | |
| | NO: 3168) | | | |
| 10 | Y180/F404 | (SEQ ID | 2900-A02 | LP3 |
| | (SEQ ID | NO: 3179) | | |
| | NO: 3168) | | | |
| 11 | Y180/F404 | (SEQ ID | 2900-A02 | LP4 |
| | (SEQ ID | NO: 3179) | | |
| | NO: 3168) | | | |
| 12 | Y180/F404 | (SEQ ID | 2900-A02 | LP5 |
| | (SEQ ID | NO: 3179) | | |
| | NO: 3168) | | | |
| 18 | Y180/F404 | (SEQ ID | 2842-B01 | LP3 |
| | (SEQ ID | NO: 3180) | HC3/LC2 | |
| | NO: 3169) | | | |
| 19 | Y180/F404 | (SEQ ID | 2842-B01 | LP4 |
| | (SEQ ID | NO: 3180) | HC3/LC2 | |
| | NO: 3169) | | | |
| 20 | Y180/F404 | (SEQ ID | 2842-B01 | LP5 |
| | (SEQ ID | NO: 3180) | HC3/LC2 | |
| | NO: 3169) | | | |
| 21 | Y180/F404 | (SEQ ID | 2900-C11 | LP1 |
| | (SEQ ID | NO: 3179) | | |
| | NO: 3170) | | | |
| 22 | Y180/F404 | (SEQ ID | 2900-F06 | LP1 |
| | (SEQ ID | NO: 3179) | | |
| | NO: 3171) | | | |
| 23 | Y180/F404 | (SEQ ID | 2900-F09 | LP1 |
| | (SEQ ID | NO: 3179) | | |
| | NO: 3172) | | | |
| 24 | Y180/F404 | (SEQ ID | 2900-H06 | LP1 |
| | (SEQ ID | NO: 3179) | | |
| | NO: 3173) | | | |
| 25 | Y180/F404 | (SEQ ID | 2900-H09 | LP1 |
| | (SEQ ID | NO: 3179) | | |
| | NO: 3174) | | | |
| 26 | Y180/F404 | (SEQ ID | 2842-B01 | LP1 |
| | (SEQ ID | NO: 3180) | HC3/LC2 | |
| | NO: 3169) | | | |
| 27 | Y180/F404 | (SEQ ID | 2842-B01 | LP1 |
| | (SEQ ID | NO: 3181) | HC3/LC1 | |
| | NO: 3169) | | | |
| 28 | Y180/F404 | (SEQ ID | 2842-B01 | LP1 |
| | (SEQ ID | NO: 3182) | HC1/LC3 | |
| | NO: 3175) | | | |
| 29 | Y180/F404 | (SEQ ID | 2842-G04 | LP1 |
| | (SEQ ID | NO: 3183) | HC2/LC2 | |
| | NO: 3176) | | | |
| 30 | Y180/F404 | (SEQ ID | 2799-A05 | LP1 |
| | (SEQ ID | NO: 3179) | | |
| | No: 3163) | | | |
| 31 | Y180/F404 | (SEQ ID | 2799-B03 | LP1 |
| | (SEQ ID | NO: 3179) | | |
| | NO: 3164) | | | |
| 32 | Y180/F404 | (SEQ ID | 2799-B06 | LP1 |
| | (SEQ ID | NO: 3179) | | |
| | NO: 3165) | | | |
| 35 | Y180/F241/ | (SEQ ID | 2900-A02 | LP2 |
| | Y391/F404 | NO: 3179) | | |
| | (SEQ ID | | | |
| | NO: 3191) | | | |

### Example 9: Synethic Example LP1 (DBCO-VALCIT-PAB-HEMIASTERLIN)

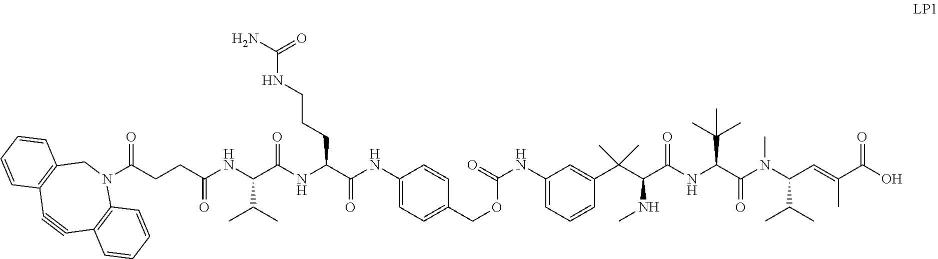 [SMILES: CN[C@H](C(=O)N[C@H](C(=O)N(C)[C@H](/C=C(\C)C(=O)O)C(C)C)C(C)(C)C)C(C)(C)c1cccc(NC(=O)OCc2ccc(NC(=O)[C@H](CCCNC(N)=O)NC(=O)[C@@H](NC(=O)CCC(=O)N3Cc4ccccc4C#Cc4ccccc43)C(C)C)cc2)c1]

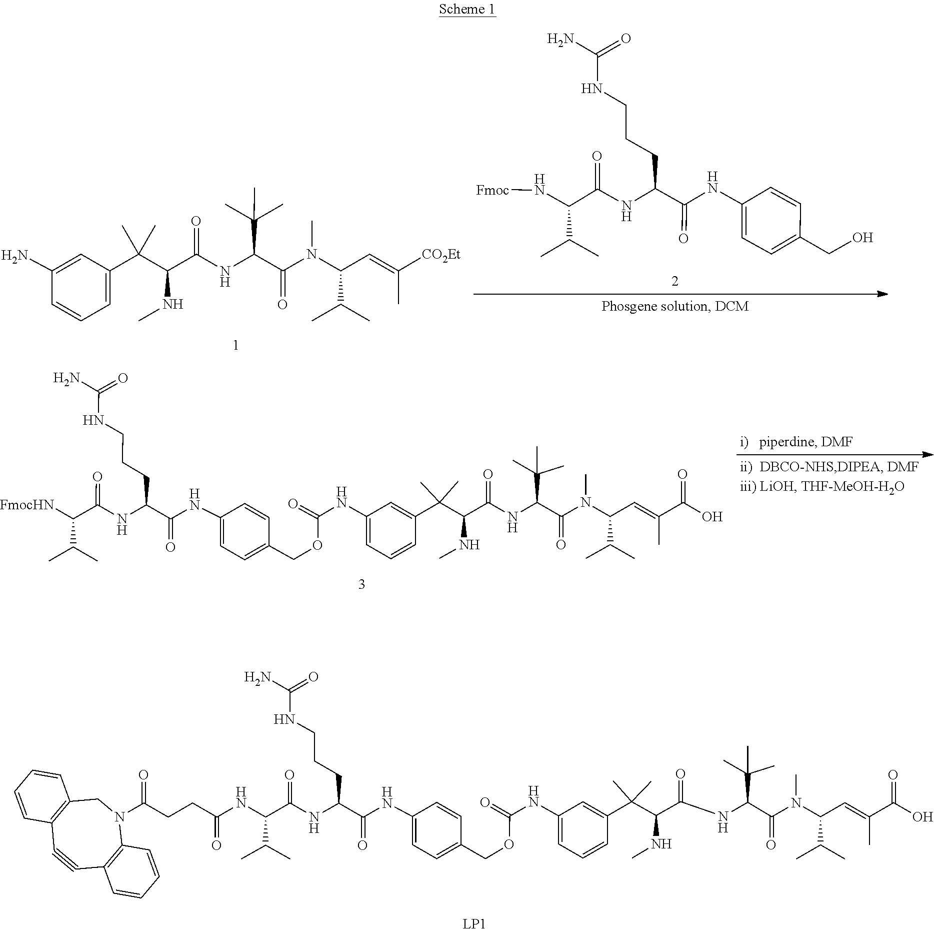

LP1 DBCO-Valcit-pAB-hemiasterlin Linker payload is synthesized as described in PCT/US2016/15844, WO 2020/252015 A1, the contents of which are hereby incorporated by reference in their entirety.

### Example 10: Synthetic Example LP2 (DBCO-BETA-GLUCURONIDE-PEG12-Exatecan) (PEGYLATED-DBCO-BETA-GLUCURONIDASE Cleavable Exatecan Linker Payload)

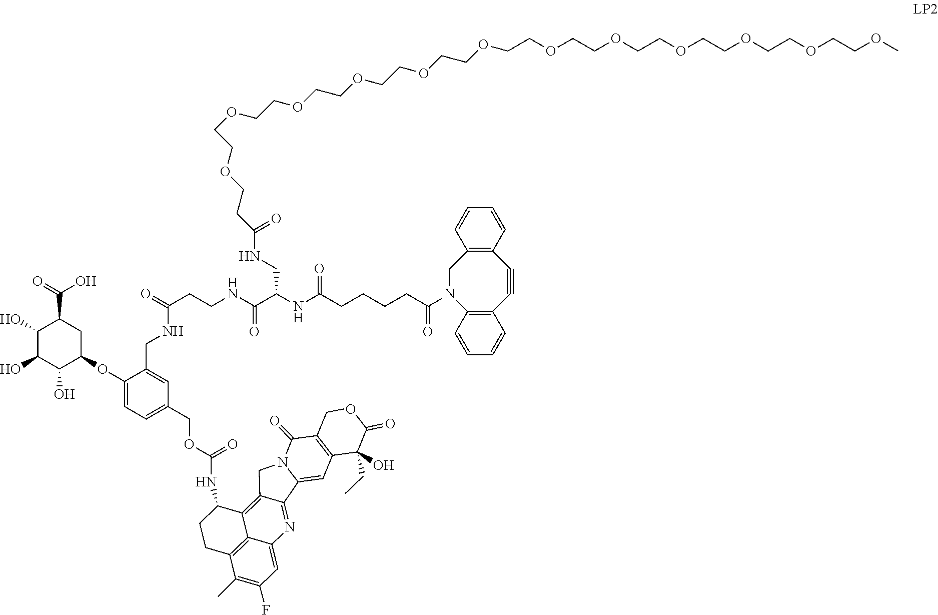 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2C[C@H](C(=O)O)[C@@H](O)[C@H](O)[C@H]2O)c(CNC(=O)CCNC(=O)[C@H](CNC(=O)CCOCCOCCOCCOCCOCCOCCOCCOCCOCCOCCOCCOC)NC(=O)CCCCC(=O)N2Cc4ccccc4C#Cc4ccccc42)c1)CC3]

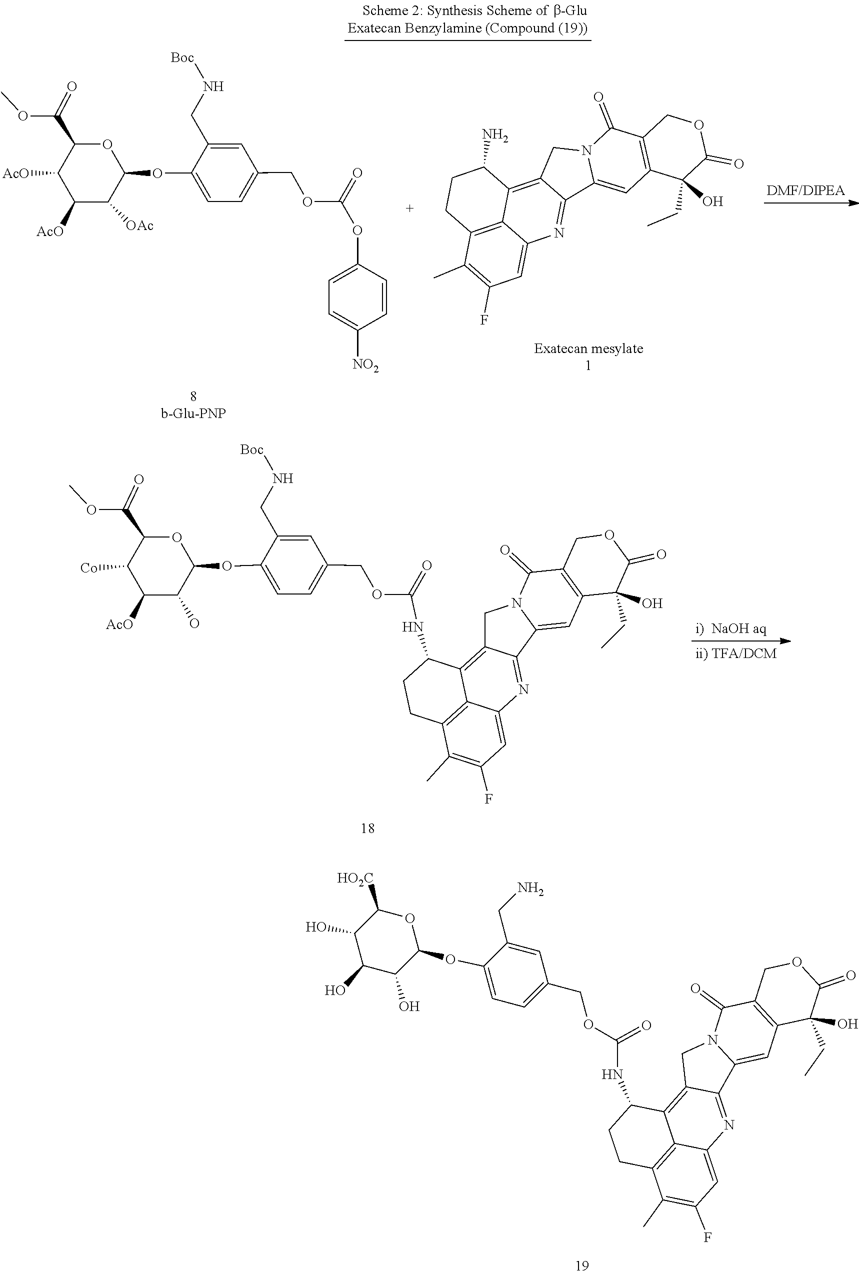

### Synthetic Scheme for Compound DBCO-B-Glu-PEG12-Exatecan (LP2)

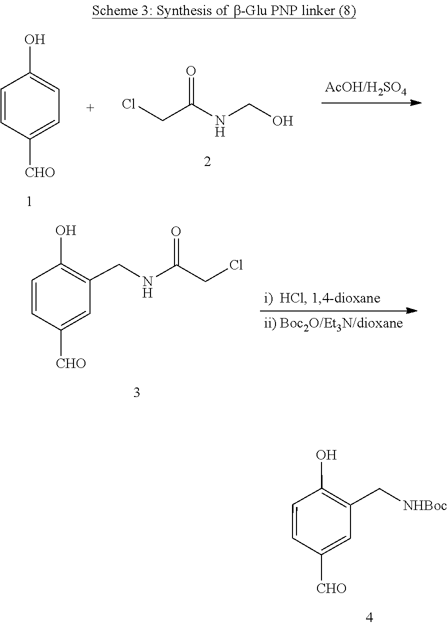 [SMILES: O=Cc1ccc(O)cc1 ; O=C(CCl)NCO ; O=Cc1ccc(O)c(CNC(=O)CCl)c1 ; CC(C)(C)OC(=O)NCc1cc(C=O)ccc1O]

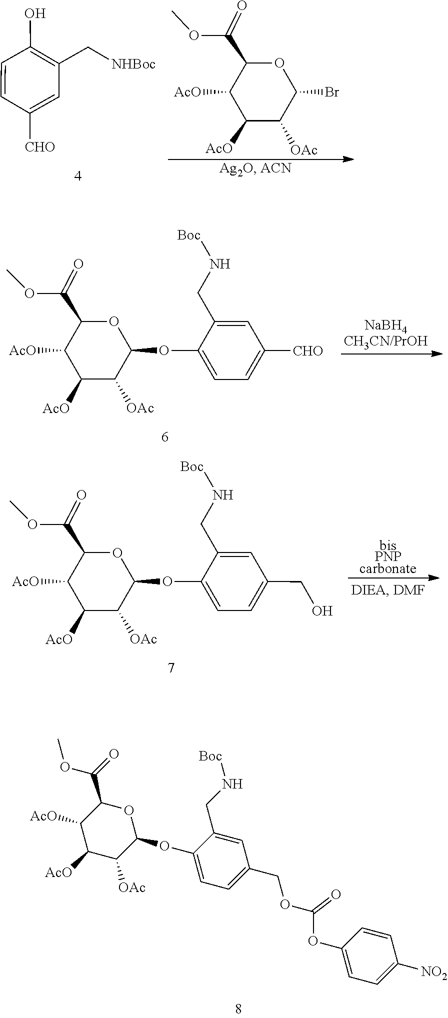

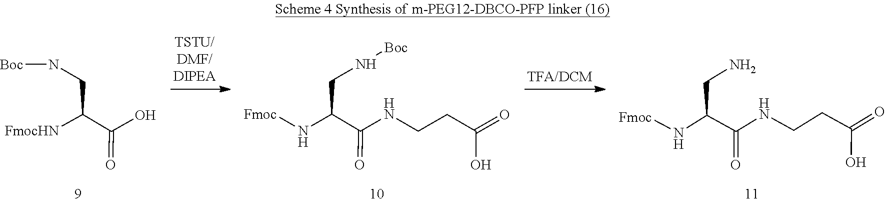

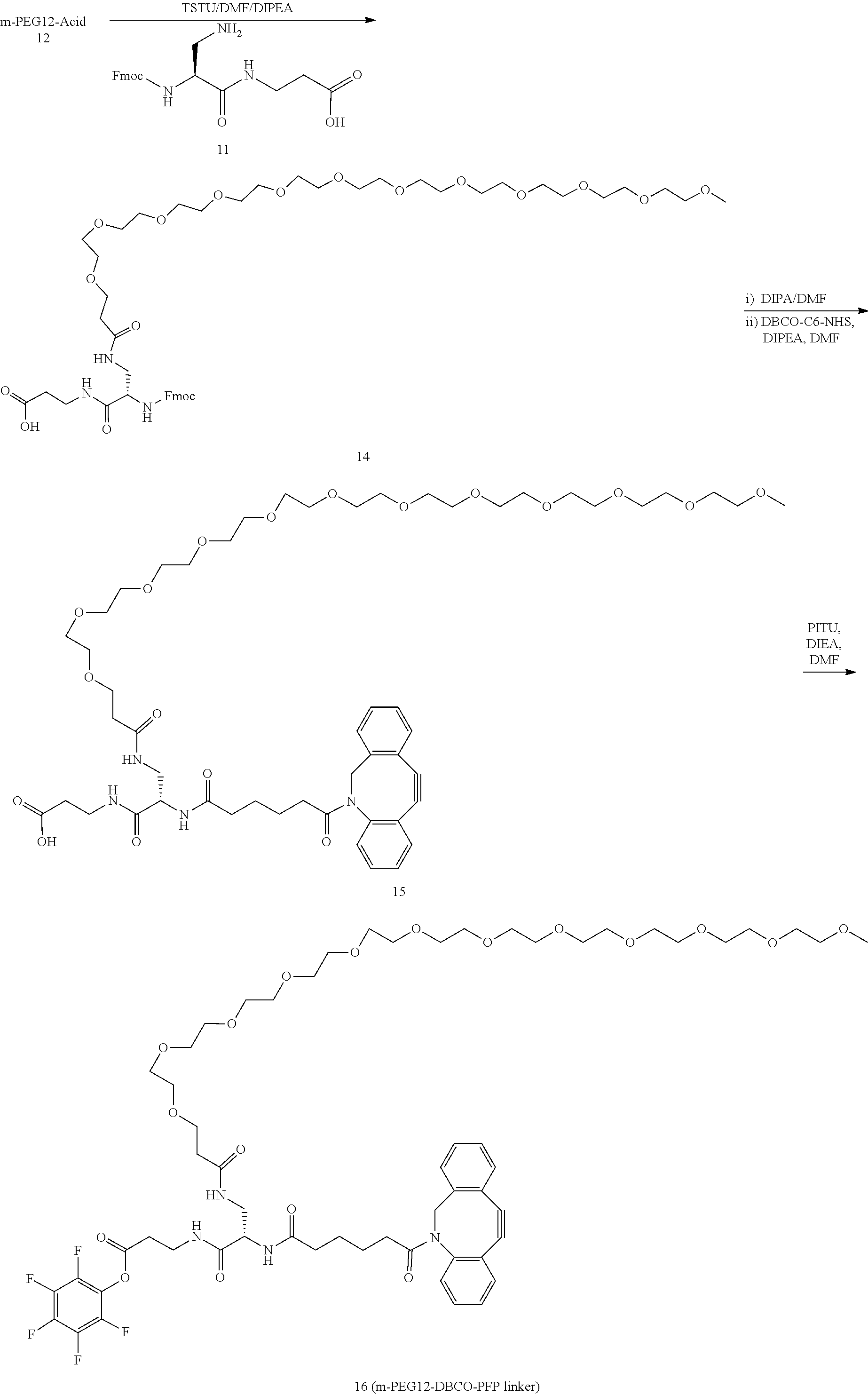

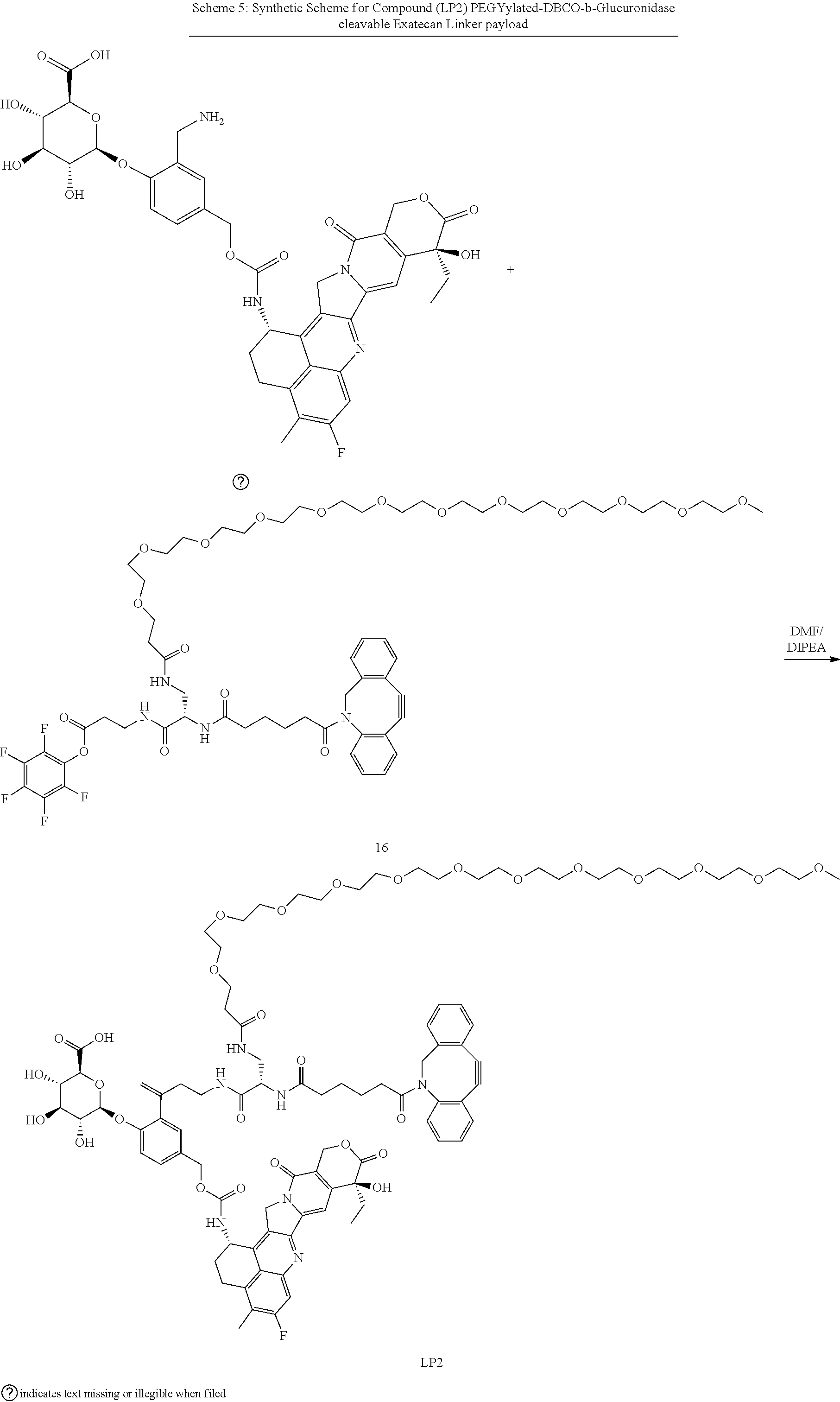 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CN)c1)CC3 ; COCCOCCOCCOCCOCCOCCOCCOCCOCCOCCOCCOCCC(=O)NC[C@H](NC(=O)CCCCC(=O)N1Cc2ccccc2C#Cc2ccccc21)C(=O)NCCC(=O)Oc1c(F)c(F)c(F)c(F)c1F ; C=C(CCNC(=O)[C@H](CNC(=O)CCOCCOCCOCCOCCOCCOCCOCCOCCOCCOCCOCCOC)NC(=O)CCCCC(=O)N1Cc2ccccc2C#Cc2ccccc21)c1cc(COC(=O)N[C@H]2CCc3c(C)c(F)cc4nc5c(c2c34)Cn2c-5cc3c(c2=O)COC(=O)[C@]3(O)CC)ccc1O[C@@H]1O[C@H](C(=O)O)[C@@H](O)[C@H](O)[C@H]1O]

### Synthesis of β-Glu PNP Linker Fragment (8)

### Synthesis of Compound (4)

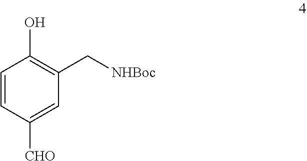 [SMILES: CC(C)(C)OC(=O)NCc1cc(C=O)ccc1O]

To a suspension of 4-hydroxybenzaldehyde (1) (5.3 g, 43.4 mmol) and 2-Chloro-N-(hydroxymethyl) acetamide (2) (5 g, 40.6 mmol) in AcOH (20 mL) was slowly added concentrated H₂SO₄ (32 mL). The mixture was stirred at room temperature (22° C.) for 16 h. The resulting viscous liquid was poured into ice water (300 mL) and extracted with EtOAc (100 mL×5). The organic layer was dried over Na₂SO₄ and concentrated under reduced pressure to give crude compound (3), which was dissolved in 1,4-dioxane (40 mL). To this solution was added concentrated hydrochloric acid (40 mL). The mixture was heated under reflux for one hour and then concentrated in vacuo to give a residue. The residue was dissolved in dioxane: H₂O (1:1, 50 mL). To this mixture was added Et₃N (9 mL), followed by Boc₂O (10 g, 46 mmol). The reaction mixture was stirred at room temperature for 16 h and partitioned between EtOAc (300 mL) and water (100 mL). The organic layer was dried over Na₂SO₄ and concentrated to dryness under reduced pressure. The residue was purified by silica gel chromatography (220 g column, 20-30% EtOAc: hexane in 30 mins, flow rate: 40 mL/min) to furnish compound (4) as an off-white solid (5.3 g). MS calculated for C₁₃H₁₇NO₄, 251.3; found 252.2 [M+H]⁺; ¹H NMR (500 MHZ, DMSO-d₆) δ 10.75 (s, 1H), 9.77 (s, 1H), 7.69-7.62 (m, 2H), 7.31 (t, J=6.2 Hz, 1H), 6.96 (d, J=8.1 Hz, 1H), 4.11 (d, J=6.1 Hz, 2H), 1.41 (s, 9H).

### Synthesis of Compound 7

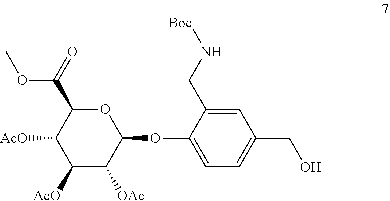 [SMILES: COC(=O)[C@H]1O[C@@H](Oc2ccc(CO)cc2CNC(=O)OC(C)(C)C)[C@H](OC(C)=O)[C@@H](OC(C)=O)[C@@H]1OC(C)=O]

To a solution of compound (4) (2.2 g, 8.8 mmol) and (2R,3R,4S,5S,6S)-2-bromo-6-(methoxycarbonyl) tetrahydro-2H-pyran-3,4,5-triyl triacetate (5) (3.2 g, 8.1 mmol) in anhydrous acetonitrile (50 mL) was added Ag₂O (3.7 g, 16 mmol). The suspension was stirred under argon atmosphere for 16 h. The solids were filtered off and washed with acetonitrile (10 mL). To the combined acetonitrile solution was added i-PrOH (10 mL) and NaBH₄ (300 mg, 8.1 mmol), and the mixture was stirred at room temperature. After 30 min, the reaction was quenched with water (100 mL) and extracted with EtOAc (3×100 mL). The organic layer was dried over Na₂SO₄ and concentrated to dryness under reduced pressure. The residue was purified by silica gel chromatography (220 g column, 40-70% EtOAc: hexane in 30 min, flow rate: 40 mL/min) to furnish compound (7) as a white foam (3.0 g, 5.2 mmol). MS calculated for C₂₆H₃₅NO₁₃, 569.2; found 570.4 [M+H]⁺; ¹H NMR (500 MHZ, DMSO-d₆) δ 7.15 (q, J=6.0 Hz, 3H), 6.98 (d, J=8.7 Hz, 1H), 5.61-5.41 (m, 2H), 5.25-5.03 (m, 3H), 4.73 (d, J=9.9 Hz, 1H), 4.42 (d, J=5.3 Hz, 2H), 4.03 (tt, J=16.4, 8.3 Hz, 2H), 3.65 (s, 3H), 2.14-1.92 (m, 10H), 1.41 (s, 9H), 1.33-1.14 (m, 2H).

### Synthesis of Compound (8)

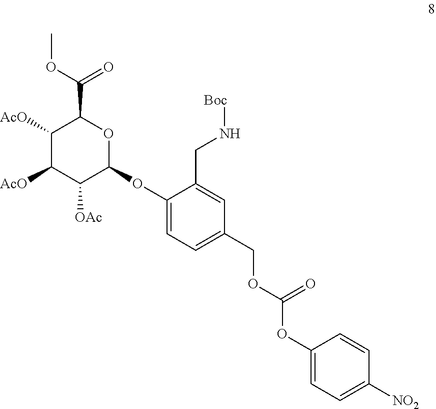 [SMILES: COC(=O)[C@H]1O[C@@H](Oc2ccc(COC(=O)Oc3ccc([N+](=O)[O-])cc3)cc2CNC(=O)OC(C)(C)C)[C@H](OC(C)=O)[C@@H](OC(C)=O)[C@@H]1OC(C)=O]

To a solution of compound (7) (2.6 g, 4.56 mmol) in anhydrous DMF (15 mL) was added DIEA (1.1 mL) and bis-PNP carbonate (2 g, 6.57 mmol). The mixture was stirred at room temperature for 16 h and purified directly by reverse phase HPLC (Phenomenex Gemini NX 5μ, C18, 110 Å, 150×50 mm, Mobile phase: A: 0.1% TFA in water, B: acetonitrile, Gradient: 20-90% B over 20 min, flow 50 mL/min) to give compound (8) (1.8 g) as a white solid after lyophilization. MS calculated for C₃₃H₃₈N₂O₁₇, 734.2; found 735.5 [M+H]⁺; ¹H NMR (500 MHz, DMSO-d₆) δ 8.35-8.29 (m, 2H), 7.60-7.53 (m, 2H), 7.36 (dd, J=8.4, 2.2 Hz, 1H), 7.29 (d, J=2.3 Hz, 1H), 7.22 (t, J=6.2 Hz, 1H), 7.09 (d, J=8.4 Hz, 1H), 5.65 (d, J=7.9 Hz, 1H), 5.52 (t, J=9.6 Hz, 1H), 5.26 (s, 2H), 5.19 (dd, J=9.8, 7.9 Hz, 1H), 5.10 (t, J=9.7 Hz, 1H), 4.76 (d, J=9.9 Hz, 1H), 4.03 (qd, J=16.7, 6.2 Hz, 3H), 3.66 (s, 3H), 2.06 (s, 3H), 2.02 (d, J=2.6 Hz, 6H), 1.39 (s, 8H), 1.30 (s, 1H).

Synthesis of m-PEG12-DBCO-PFP

### Synthesis of Compound 11

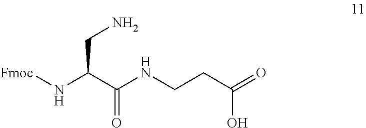

To a solution of Fmoc-Dap (Boc)-COOH (9) (853 mg, 2 mmol) in anhydrous DMF (10 mL) was added N,N,N′,N′-Tetramethyl-O—(N-succinimidyl) uronium tetrafluoroborate (TSTU) (608 mg, 2 mmol), followed by DIPEA (0.7 mL). The mixture was stirred at room temperature for 10 min. A solution of beta-alanine (0.2 g) in acetonitrile:water (1:1, 3 mL) was added, followed by DIPEA (0.4 mL). The reaction mixture was stirred at room temperature for 30 min, and then acidified with 0.5 N hydrochloric acid (50 mL). The mixture was extracted with EtOAc (200 mL) and the organic layer was dried over Na₂SO₄ and concentrated to dryness under reduced pressure to give crude compound (10) as white powder. Crude compound (10) was treated with TFA: DCM (1:4, v/v, 20 mL) at room temperature for one hour. The mixture was evaporated to dryness under reduced pressure to give crude compound (11) which was used directly in the next step. MS calculated for C₂₁H₂₃N₃O₅, 397.16; found 398.2 [M+H]⁺.

### Synthesis of Compound (14)

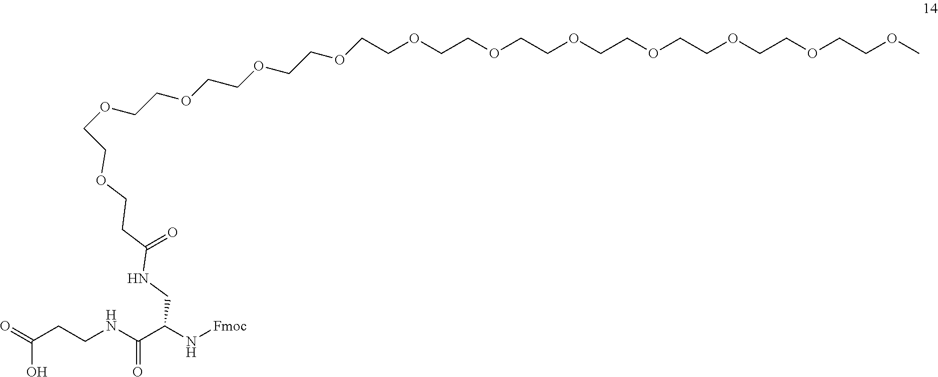

m-PEG12-acid (12) (1.18 g, 2 mmol) was dissolved in DMF (10 mL) and TSTU (610 mg, 2 mmol) was added, followed by DIPEA (700 μL). After 5 min, a solution of compound (11) in DMF (10 mL) with DIPEA (0.7 mL) was added. The reaction mixture was stirred at room temperature for 30 mins and purified directly by reverse phase HPLC to give compound (14) as a viscous foam (1.27 g). MS calculated for C₄₇H₇₃N₃O₁₈, 967.5; found 968.8 [M+H]⁺; ¹H NMR (500 MHz, DMSO-d₆) δ 8.35-8.29 (m, 2H), 7.60-7.53 (m, 2H), 7.36 (dd, J=8.4, 2.2 Hz, 1H), 7.29 (d, J=2.3 Hz, 1H), 7.22 (t, J=6.2 Hz, 1H), 7.09 (d, J=8.4 Hz, 1H), 5.65 (d, J=7.9 Hz, 1H), 5.52 (t, J=9.6 Hz, 1H), 5.26 (s, 2H), 5.19 (dd, J=9.8, 7.9 Hz, 1H), 5.10 (t, J=9.7 Hz, 1H), 4.76 (d, J=9.9 Hz, 1H), 4.03 (qd, J=16.7, 6.2 Hz, 3H), 3.66 (s, 3H), 2.06 (s, 3H), 2.02 (d, J=2.6 Hz, 6H), 1.39 (s, 8H), 1.30 (s, 1H).

### Synthesis of Compound (15)

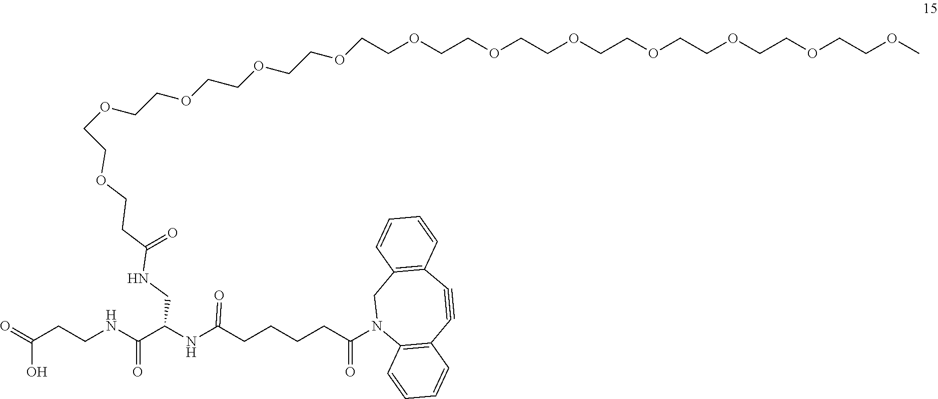 [SMILES: COCCOCCOCCOCCOCCOCCOCCOCCOCCOCCOCCOCCC(=O)NC[C@H](NC(=O)CCCCC(=O)N1Cc2ccccc2C#Cc2ccccc21)C(=O)NCCC(=O)O]

To a solution of compound (14) (1.24 g) in DMF (10 mL) was added i-Pr₂NH (DIPEA) (10 mL) and the reaction mixture was stirred at room temperature for 2 h. The mixture was then concentrated under reduced pressure to about 9 mL. To this solution, DBCO-C6-NHS (0.55 g) was added followed by DIPEA (0.23 mL). The mixture was stirred at room temperature for 2 h and then purified directly by reverse phase HPLC to give compound (15) as a colorless syrup (1.17 g). MS calculated for C53H80N4018, 1060.6; found 1061.9 [M+H]⁺.

### Synthesis of Compound (16)

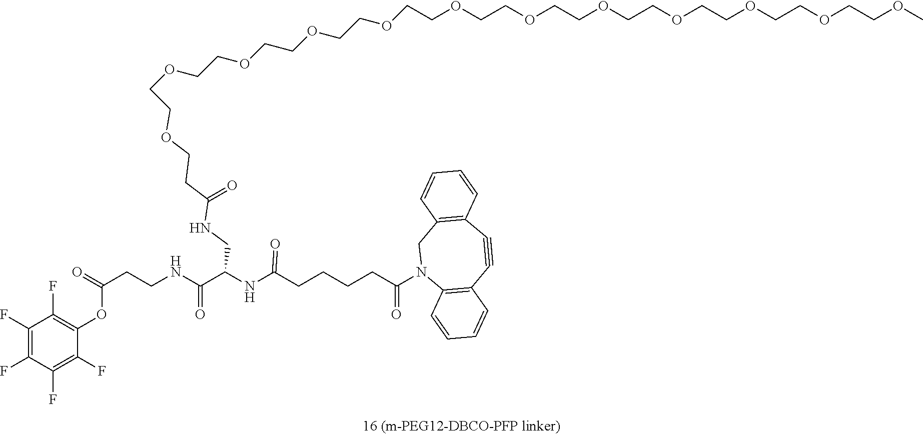 [SMILES: COCCOCCOCCOCCOCCOCCOCCOCCOCCOCCOCCOCCC(=O)NC[C@H](NC(=O)CCCCC(=O)N1Cc2ccccc2C#Cc2ccccc21)C(=O)NCCC(=O)Oc1c(F)c(F)c(F)c(F)c1F]

To a solution of compound (15) (1.1 g) in DMF (8 mL) was added Pentafluorophenol-tetramethyluronium hexafluorophosphate (PfTU) (0.5 g) followed by DIEA (0.4 mL) and the reaction mixture was stirred at room temperature for 10 min. The mixture was purified directly by RP-HPLC to give compound (16) as a colorless syrup (1.0 g). MS calculated for C₅₉H₇₉F₅N₄O₁₈, 1226.5; found 1227.8 [M+H]⁺. Compound (16) was used in the next reaction.

### Synthesis of Compound (19)

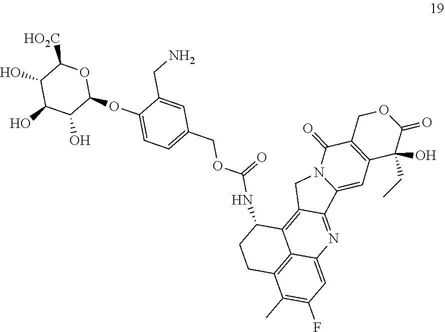 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CN)c1)CC3]

To a solution of compound (8) (735 mg, 1 mmol) and Exatecan mesylate 1, 531 mg, 1 mmol) in DMF (10 mL) was added DIPEA (350 μL). The reaction mixture was stirred at room temperature (22° C.) for 5 hours and then diluted with EtOAc (200 mL). The mixture was washed with 0.5 N hydrochloric acid (100 mL), water (100 mL), and brine (50 mL). The organic layer was dried over Na₂SO₄ and concentrated to dryness under reduced pressure to give crude compound (18) which was suspended in acetonitrile:water (2:1, 50 mL). To this mixture, 1 N aq. NaOH (7 mL) was added and the reaction was stirred at room temperature. After 3 h, 1 N hydrochloric acid (7 mL) was added and the mixture was evaporated to dryness under reduced pressure. The resulting residue was treated with TFA: DCM (1:4, v/v, 20 mL) at room temperature for one hour. The mixture was diluted with toluene (30 mL) and then evaporated to dryness under reduced pressure. The residue was purified by reverse phase HPLC to give compound (19) as a yellow solid (745 mg). MS calculated for C₃₉H₃₉FN₄O₁₃, 790.3; found 791.6 [M+H]⁺; ¹H NMR (500 MHZ, DMSO-d₆) δ 8.09 (d, J=8.0 Hz, 4H), 7.73 (dd, J=10.7, 3.2 Hz, 1H), 7.54 (d, J=2.1 Hz, 1H), 7.49 (dd, J=8.5, 2.2 Hz, 1H), 7.32 (s, 1H), 7.22 (d, J=8.5 Hz, 1H), 6.55 (s, 1H), 5.75 (s, 1H), 5.48 (d, J=16.3 Hz, 1H), 5.46-5.37 (m, 3H), 5.31-5.13 (m, 4H), 5.08-5.02 (m, 2H), 4.19-4.07 (m, 2H), 3.95 (d, J=9.5 Hz, 1H), 3.44 (dd, J=8.8, 2.6 Hz, 1H), 3.28-3.19 (m, 2H), 3.14-3.04 (m, 1H), 2.34 (s, 3H), 2.26 (dd, J=12.5, 6.4 Hz, 1H), 2.12 (qd, J=9.0, 4.6 Hz, 1H), 1.87 (dh, J=21.5, 7.2 Hz, 2H), 0.89 (t, J=7.2 Hz, 3H).

### Final Coupling of Synthesis LP2:

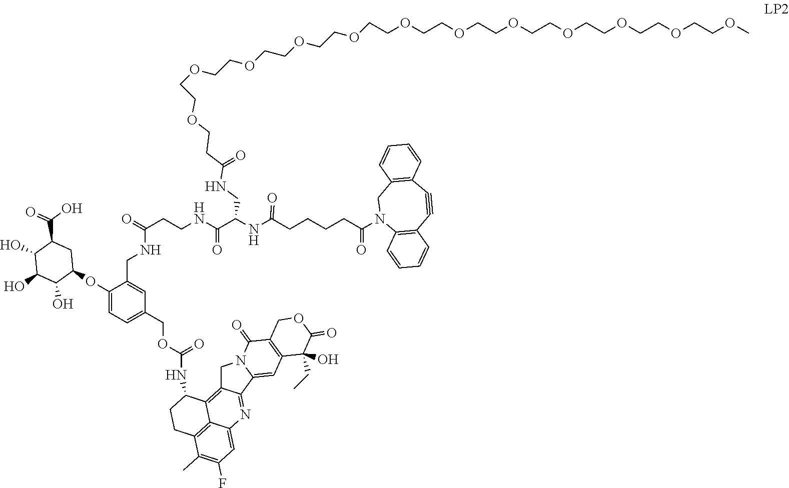 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2C[C@H](C(=O)O)[C@@H](O)[C@H](O)[C@H]2O)c(CNC(=O)CCNC(=O)[C@H](CNC(=O)CCOCCOCCOCCOCCOCCOCCOCCOCCOCCOCCOCCOC)NC(=O)CCCCC(=O)N2Cc4ccccc4C#Cc4ccccc42)c1)CC3]

The compound b-Glu-Exatecan benzyl amine (19) (540 mg, 0.68 mmol) was dissolved in DMF (4 mL) and compound m-PEG12-DBCO-PFP (16) (750 mg, 0.6 mmol) was added, followed by DIPEA (0.32 mL). The reaction mixture was stirred at room temperature for 30 min and purified directly by reverse phase HPLC (Phenomenex Gemini NX 5μ, C18, 110 Å, 150×50 mm, Mobile phase: A: 0.1% TFA in water, B: acetonitrile, Gradient: 20-90% B over 20 min, flow 50 mL/min) to give compound LP2 as a pale-yellow solid (704 mg). HRMS m/z (ESI⁺): calculated for C₉₂H₁₁₇FN₈O₃₀, 1832.78; found 1833.79 [M+H]⁺; ¹H NMR (500 MHZ, DMSO-d₆) § 12.84 (s, 1H), 8.20 (t, J=6.2 Hz, 1H), 8.02 (d, J=8.8 Hz, 1H), 7.75 (td, J=6.1, 3.4 Hz, 2H), 7.62-7.52 (m, 1H), 7.50-7.39 (m, 2H), 7.32-7.25 (m, 2H), 6.52 (s, 1H), 5.50-5.39 (m, 2H), 5.27 (d, J=13.6 Hz, 3H), 5.01-4.94 (m, 1H), 4.29 (t, J=6.3 Hz, 2H), 3.57 (d, J=14.0 Hz, 1H), 3.56-3.46 (m, 35H), 3.48-3.40 (m, 5H), 3.35 (td, J=12.8, 6.5 Hz, 5H), 3.24 (s, 2H), 3.22 (s, 3H), 2.36 (d, J=1.8 Hz, 3H), 2.27 (dq, J=13.4, 6.3 Hz, 3H), 2.16 (ddd, J=21.5, 11.0, 6.0 Hz, 2H), 1.87 (dp, J=21.0, 7.2 Hz, 4H), 1.17 (s, 3H), 0.88 (t, J=7.3 Hz, 3H).

### Example 11: Synthetic Example LP3 (DBCO-NNAA-PEG13-VKG-EXATECAN)

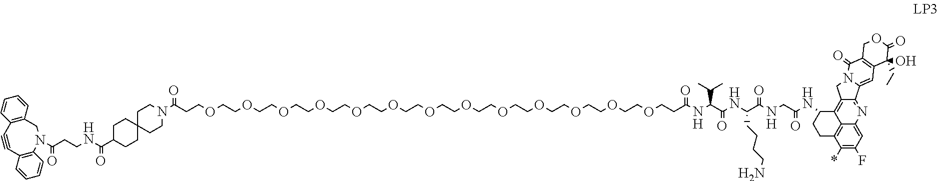 [SMILES: *c1c(F)cc2nc3c(c4c2c1CC[C@@H]4NC(=O)CNC(=O)[C@H](CCCCN)NC(=O)[C@@H](NC(=O)CCOCCOCCOCCOCCOCCOCCOCCOCCOCCOCCOCCOCCOCCC(=O)N1CCC2(CCC(C(=O)NCCC(=O)N4Cc5ccccc5C#Cc5ccccc54)CC2)CC1)C(C)C)Cn1c-3cc2c(c1=O)COC(=O)[C@]2(O)CC]

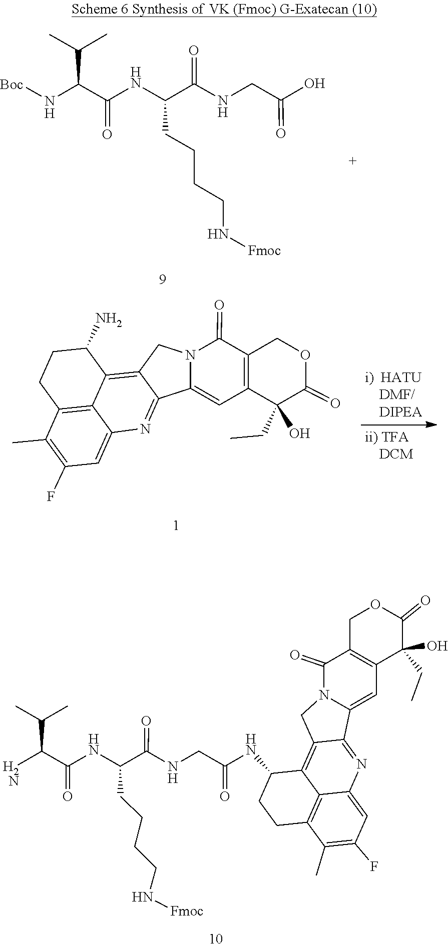

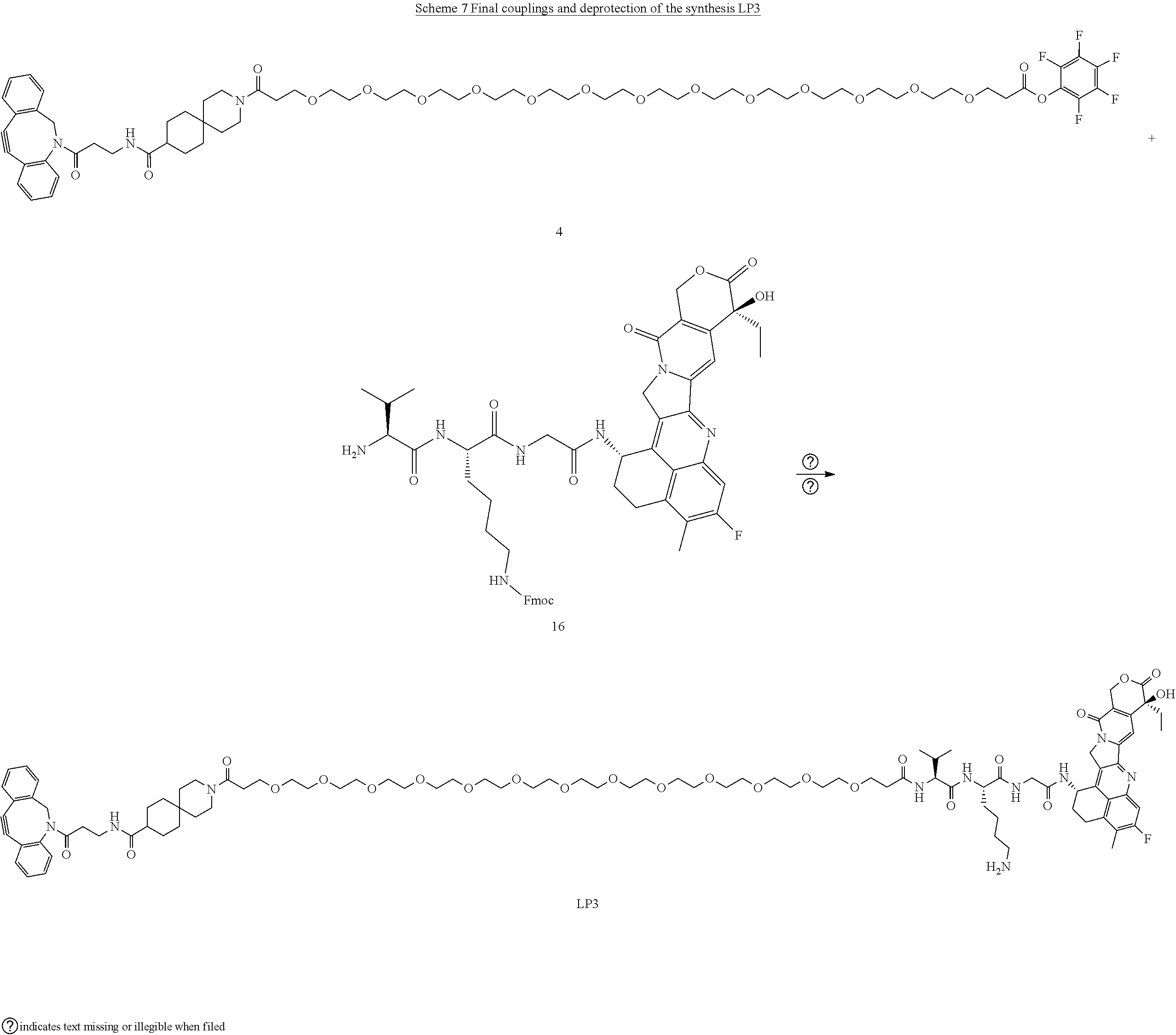

### Synthesis of Compound 10

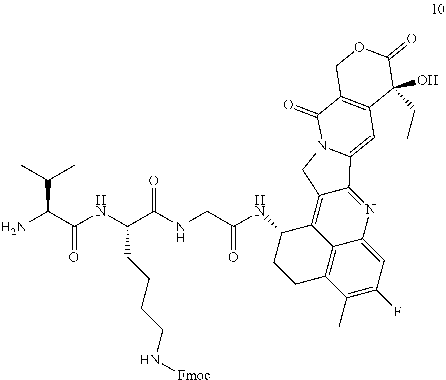

To a solution of compound Boc-VK (Fmoc)-G-OH 9 (562 mg, 0.9 mmol) in anhydrous DMF (10 mL) was added Exatecan mesylate (1) (478 mg, 0.9 mmol) and DIPEA (470 μL). After all components dissolved, HATU (342 mg, 0.9 mmol) was added, and the mixture was stirred at room temperature for 10 min. The reaction was then diluted with water (80 mL) and extracted with EtOAc (150 mL). The organic layer was washed with hydrochloric acid (0.2 M, 50 mL) and brine (50 mL), dried over Na₂SO₄ and evaporated to dryness under reduced pressure. The residue obtained was treated with TFA/DCM (¼, 20 mL) at room temperature for 30 min and the reaction was evaporated to dryness under reduced pressure. The crude mixture was dissolved in 5 mL of DMF and purified by reverse phase HPLC to give compound 10 as a yellowish solid (771 mg). MS calculated for C₅₂H₅₆FN₇O₉, 941.4; found 942.9 [M+H]⁺.

### Synthesis of LP3

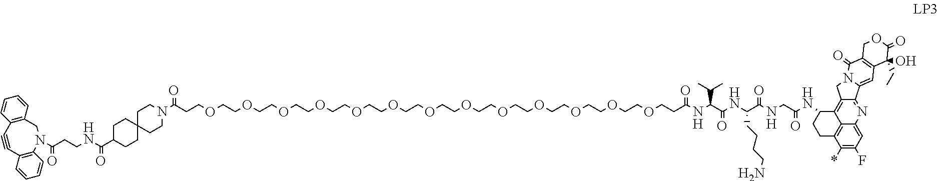 [SMILES: *c1c(F)cc2nc3c(c4c2c1CC[C@@H]4NC(=O)CNC(=O)[C@H](CCCCN)NC(=O)[C@@H](NC(=O)CCOCCOCCOCCOCCOCCOCCOCCOCCOCCOCCOCCOCCOCCC(=O)N1CCC2(CCC(C(=O)NCCC(=O)N4Cc5ccccc5C#Cc5ccccc54)CC2)CC1)C(C)C)Cn1c-3cc2c(c1=O)COC(=O)[C@]2(O)CC]

To a solution of compound 4 (426 mg, 0.33 mmol) in anhydrous DMF (3 mL) was added compound 10 (TFA salt, 347 mg, 0.33 mmol) and DIPEA (130 μL). The mixture was stirred at room temperature for 15 min, the LCMS showed the desired product formation. Then DBU (366 μL) was added dropwise, and the mixture was stirred at room temperature for additional 10 min. LCMS showed completion of the reaction. Then the mixture was purified directly by reverse phase HPLC to give LP3 as a yellowish solid (350 mg); HRMS m/z (ESI⁺): calculated for C₉₆H₁₃₃FN₁₀O₂₄, 1828.94; found 1829.95 [M+H]⁺; ¹H NMR (500 MHZ, DMSO) δ 8.43 (d, J=8.5 Hz, 1H), 8.14 (t, J=5.6 Hz, 1H), 7.98 (d, J=7.4 Hz, 1H), 7.81 (d, J=10.9 Hz, 1H), 7.77-7.53 (m, 7H), 7.54-7.23 (m, 10H), 5.57 (dt, J=8.7, 4.4 Hz, 1H), 5.47-5.38 (m, 2H), 5.25 (d, J=3.3 Hz, 2H), 5.04 (d, J=14.1 Hz, 2H), 4.16 (td, J=8.1, 5.9 Hz, 3H), 4.08 (dd, J=8.5, 6.8 Hz, 3H), 3.88-3.68 (m, 13H), 3.50 (d, J=3.6 Hz, 71H), 3.25-3.13 (m, 4H), 3.13-3.03 (m, 2H), 2.92 (dq, J=13.4, 6.8 Hz, 2H), 2.78 (q, J=6.8 Hz, 3H), 2.60-2.26 (m, 18H), 2.26-2.03 (m, 3H), 1.98-1.74 (m, 6H), 1.74-1.46 (m, 8H), 1.46-1.09 (m, 12H), 0.97 (s, 2H), 0.88 (t, J=7.3 Hz, 3H), 0.78 (dd, J=14.1, 6.7 Hz, 7H).

### Example 12: Synthetic Example LP4 (DBCO-NNAA-PEG13-AAN-EXATECAN) Legumain Cleavable Exatecan Linker Payload

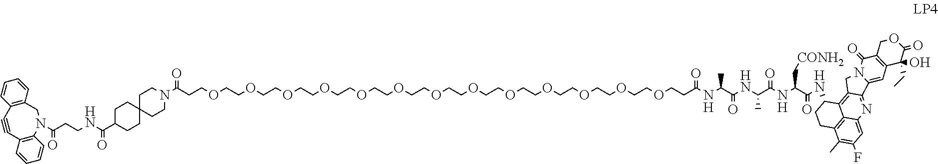 [SMILES: CC[C@@]1(O)C(=O)OCc2c1cc1n(c2=O)Cc2c-1nc1cc(F)c(C)c3c1c2[C@@H](NC(=O)[C@H](CC(N)=O)NC(=O)[C@H](C)NC(=O)[C@H](C)NC(=O)CCOCCOCCOCCOCCOCCOCCOCCOCCOCCOCCOCCOCCOCCC(=O)N1CCC2(CCC(C(=O)NCCC(=O)N4Cc5ccccc5C#Cc5ccccc54)CC2)CC1)CC3]

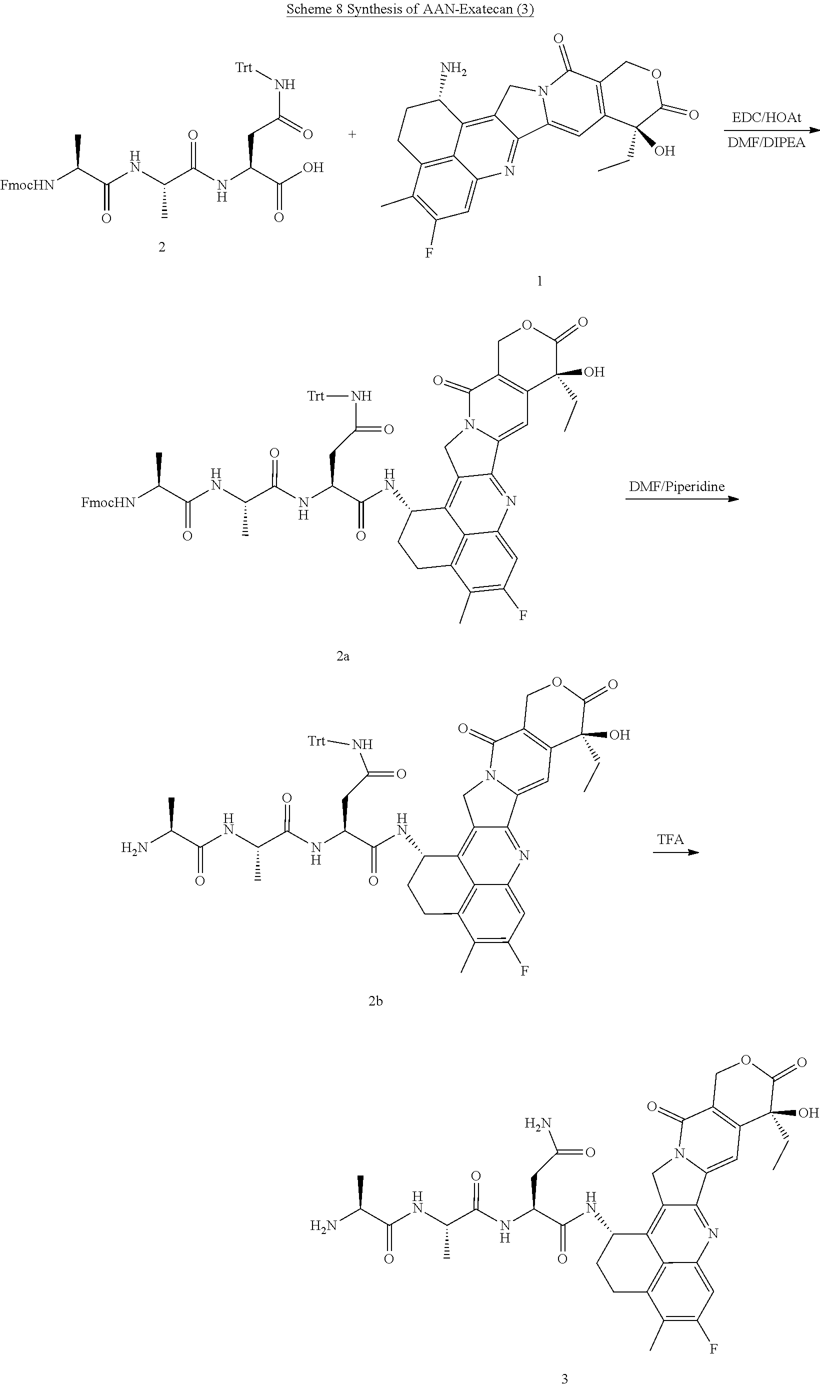

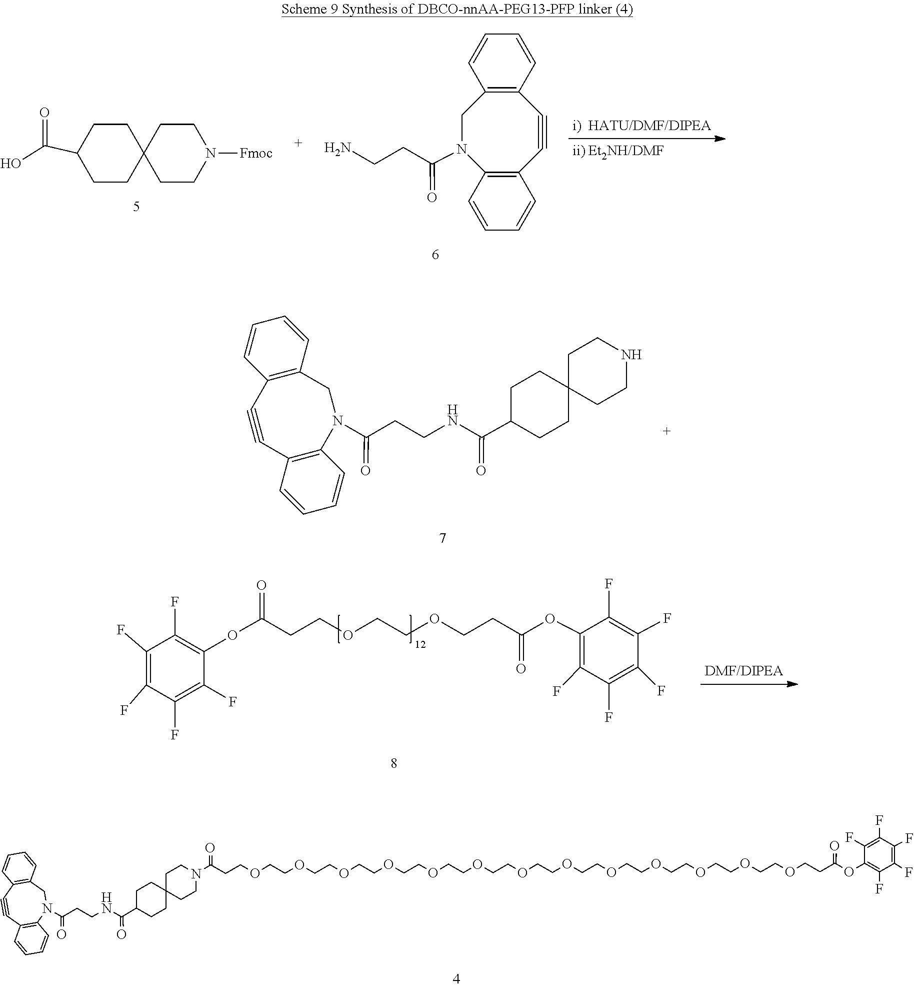

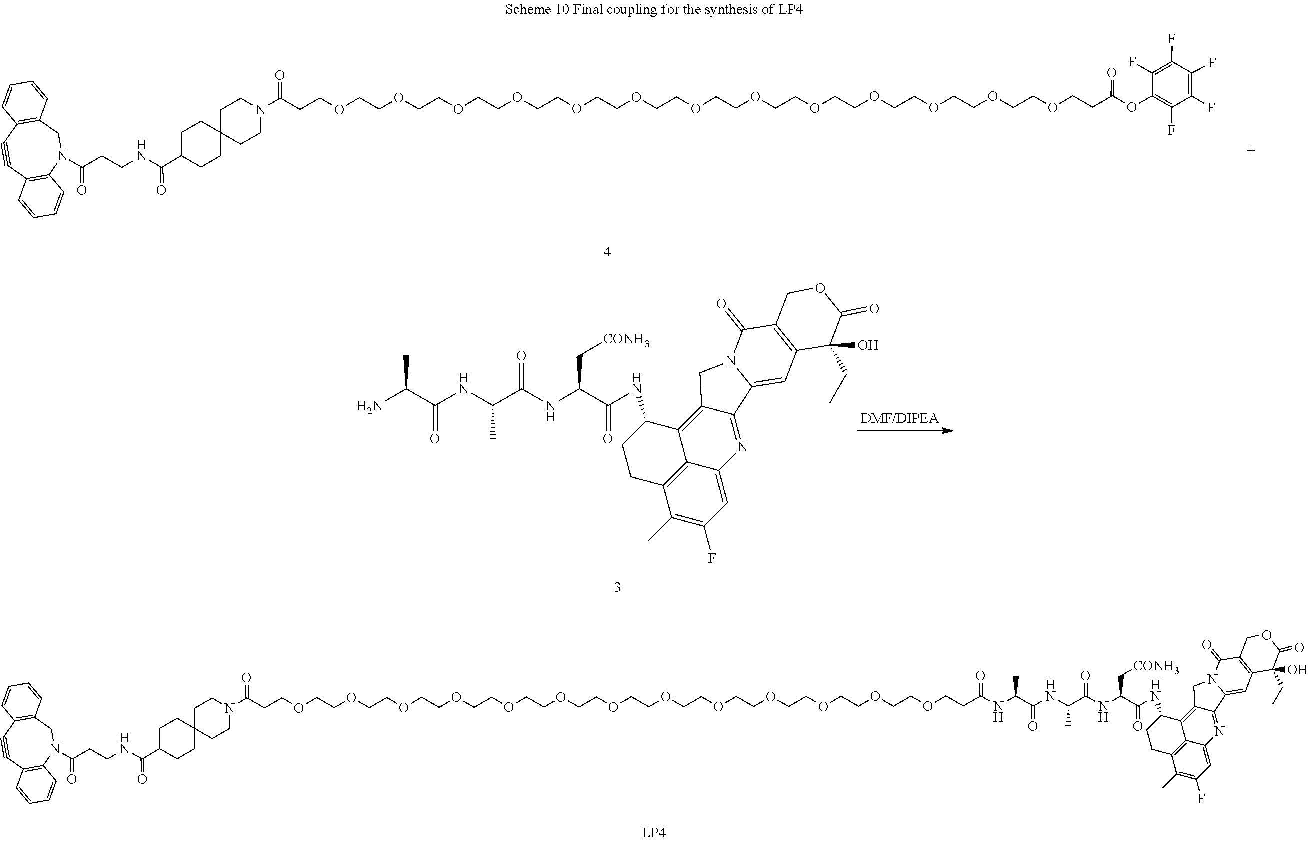

### Synthesis of Compound 3:

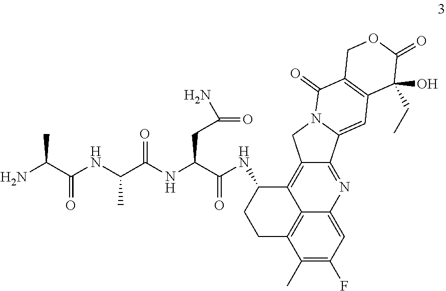 [SMILES: CC[C@@]1(O)C(=O)OCc2c1cc1n(c2=O)Cc2c-1nc1cc(F)c(C)c3c1c2[C@@H](NC(=O)[C@H](CC(N)=O)NC(=O)[C@H](C)NC(=O)[C@H](C)N)CC3]

To a suspension of exatecan mesylate 1 (MsOH salt, 150 mg, 0.28 mmol) in anhydrous DMF (3 mL) at room temperature was added Fmoc-AAN (Trt)-OH 2 (250 mg, 0.33 mmol), EDC (65 mg, 0.34 mmol), HOAt (46 mg, 0.34 mmol), and DIPEA (53 μL). The reaction was stirred at RT for 1 h, LCMS showed the desired product, then 0.3 mL of piperidine was added. The mixture was stirred for 5 min, and then added to a 1/1 mixture of hexane/diethyl ether (45 mL). The precipitate was collected by centrifugation and the solvent was removed by decantation to give the compound 2b. The residue 2b was dissolved in 3 mL of TFA and the mixture was stirred for 10 min at RT. Then the TFA was removed under reduced pressure, the crude mixture was purified by reverse phase HPLC. Pure Fractions were lyophilized to give compound 3 as a TFA salt (63 mg); LCMS m/z (ESI⁺): calculated for C₃₄H₃₈FN₇O₈, 691.28; found 692.4 (M+H).

Synthesis of compound 7:

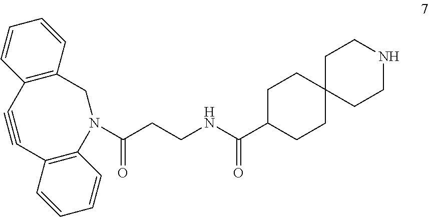 [SMILES: O=C(NCCC(=O)N1Cc2ccccc2C#Cc2ccccc21)C1CCC2(CCNCC2)CC1]

Compound 5 (1.4 g, 3.34 mmol) was dissolved in DMF (10 mL). To the clear solution was added HATU (1.2 g, 3.34 mmol) and DIPEA (861 mg, 6.68 mmol). The solution was stirred at room temperature for about 30 sec. followed by the addition of DBCO-amine 7 (922 mg, 3.34 mmol) in DMF (2 mL). After the mixture was stirred at room temperature for 20 minutes, diethyl amine (2 mL) was added into and allowed to stir for another 30 minutes, LCMS showed completion of the reaction. The reaction solution was concentrated under reduced pressure and purified by reverse phase HPLC to afford compound 7 (790 mg); LCMS m/z (ESI⁺): calculated for C₂₉H₃₃N₃O₂, 455.26; found 456.4 (M+H).

Synthesis of compound 4:

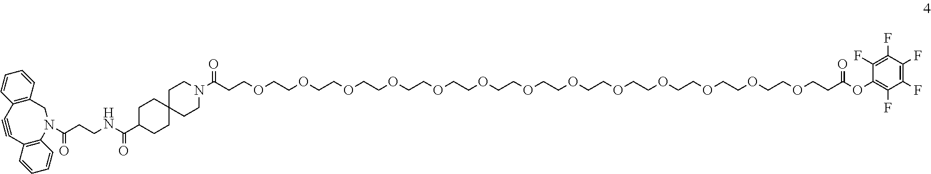 [SMILES: O=C(CCOCCOCCOCCOCCOCCOCCOCCOCCOCCOCCOCCOCCOCCC(=O)N1CCC2(CCC(C(=O)NCCC(=O)N3Cc4ccccc4C#Cc4ccccc43)CC2)CC1)Oc1c(F)c(F)c(F)c(F)c1F]

A solution of compound 8 (3 g, 2.9 mmol), compound 7 (780 mg, 1.37 mmol) and DIPEA (353 mg, 2.74 mmol) in DMF (20 mL) was stirred for 20 minutes, LCMS showed completion of the reaction. The reaction mixture was directly purified by reverse phase HPLC to give compound 4 (1.52 g); LCMS m/z (ESI⁺): calculated for C₆₅H₈₈F₅N₃O₁₈, 1293.60; found 1293.7 (M+H).

### Synthesis of LP4:

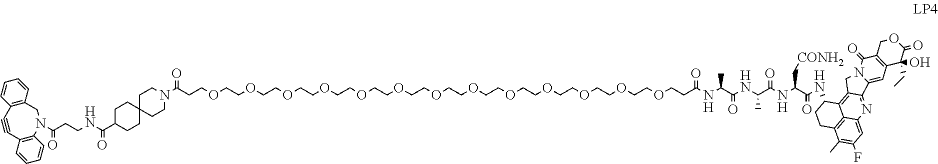 [SMILES: CC[C@@]1(O)C(=O)OCc2c1cc1n(c2=O)Cc2c-1nc1cc(F)c(C)c3c1c2[C@@H](NC(=O)[C@H](CC(N)=O)NC(=O)[C@H](C)NC(=O)[C@H](C)NC(=O)CCOCCOCCOCCOCCOCCOCCOCCOCCOCCOCCOCCOCCOCCC(=O)N1CCC2(CCC(C(=O)NCCC(=O)N4Cc5ccccc5C#Cc5ccccc54)CC2)CC1)CC3]

To a solution of compound 4 (770 mg, 0.6 mmol) in anhydrous DMF (3 mL) was added compound 3 (TFA salt, 478 mg, 0.6 mmol) and DIPEA (206 μL). The mixture was stirred at room temperature for 10 min, LCMS showed completion of the reaction. Then the mixture was purified directly by reverse phase HPLC to give LP4 as a light yellowish solid (710 mg); ¹H NMR (500 MHZ, DMSO-d₆) δ 11.41 (s, 1H), 8.25 (d, J=8.5 Hz, 1H), 8.07-7.91 (m, 3H), 7.77 (d, J=10.9 Hz, 1H), 7.68-7.54 (m, 2H), 7.47 (dddd, J=13.4, 7.6, 4.9, 2.8 Hz, 4H), 7.43-7.22 (m, 5H), 6.98-6.83 (m, 1H), 5.51 (dt, J=8.8, 4.5 Hz, 1H), 5.43 (s, 2H), 5.22 (s, 2H), 5.04 (d, J=14.0 Hz, 1H), 4.47 (q, J=6.8 Hz, 1H), 4.09 (dp, J=18.0, 7.1 Hz, 3H), 3.73 (s, 10H), 3.67-3.27 (m, 61H), 3.22-3.01 (m, 3H), 2.92 (dq, J=13.3, 6.7 Hz, 1H), 2.68-2.45 (m, 8H), 2.45-2.28 (m, 6H), 2.19 (dq, J=9.3, 4.8 Hz, 1H), 1.87 (qd, J=13.8, 7.1 Hz, 4H), 1.61 (d, J=13.1 Hz, 2H), 1.49-1.21 (m, 7H), 1.13 (dd, J=26.8, 7.1 Hz, 7H), 0.97 (ddd, J=16.1, 11.6, 6.4 Hz, 2H), 0.88 (t, J=7.3 Hz, 3H); LCMS m/z (ESI⁺): calculated for C₉₃H₁₂₅FN₁₀O₂₅, 1801.9; found 1802.9 [M+H]⁺.

### Example 13: Synthetic Example LP5 (DBCO-NNAA-PEG13-AAA-EXATECAN)

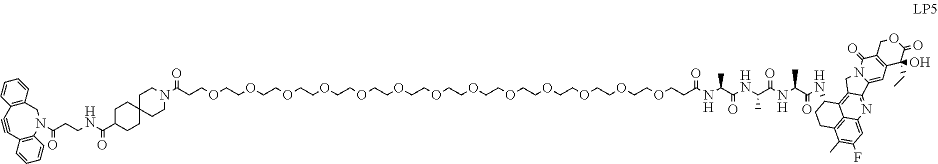 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@H](C)NC(=O)[C@H](C)NC(=O)CCOCCOCCOCCOCCOCCOCCOCCOCCOCCOCCOCCOCCOCCC(=O)N1CCC2(CCC(C(=O)NCCC(=O)N4Cc5ccccc5C#Cc5ccccc54)CC2)CC1)CC3]

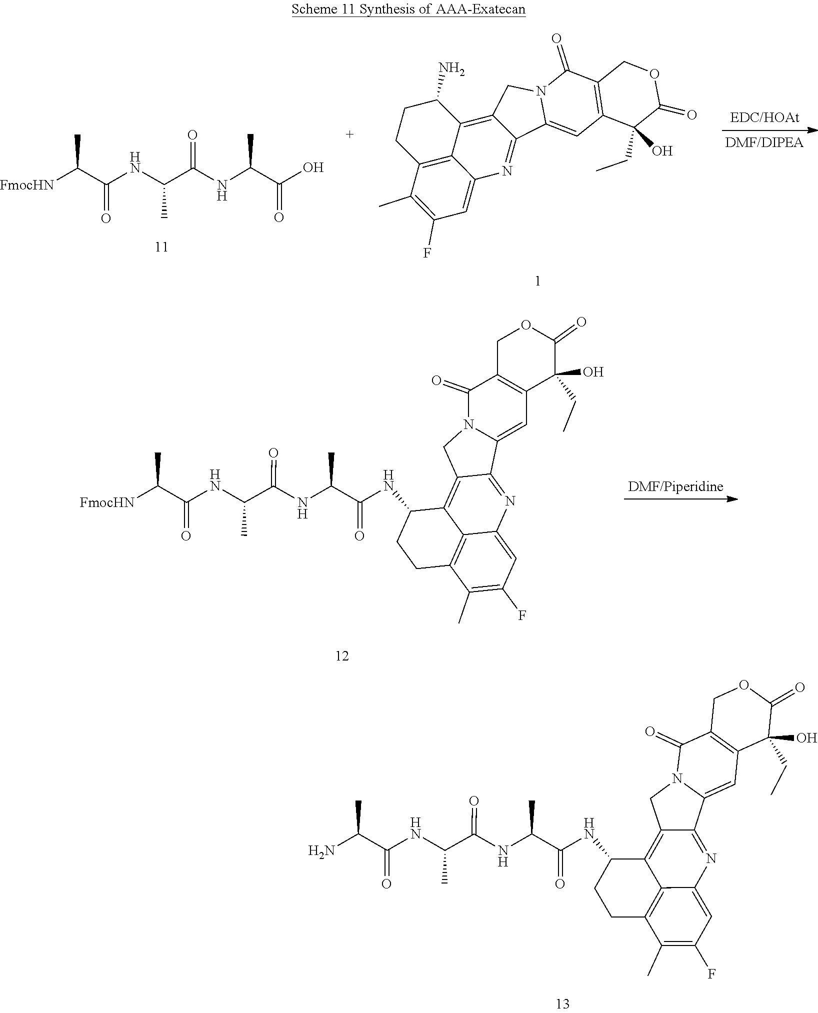

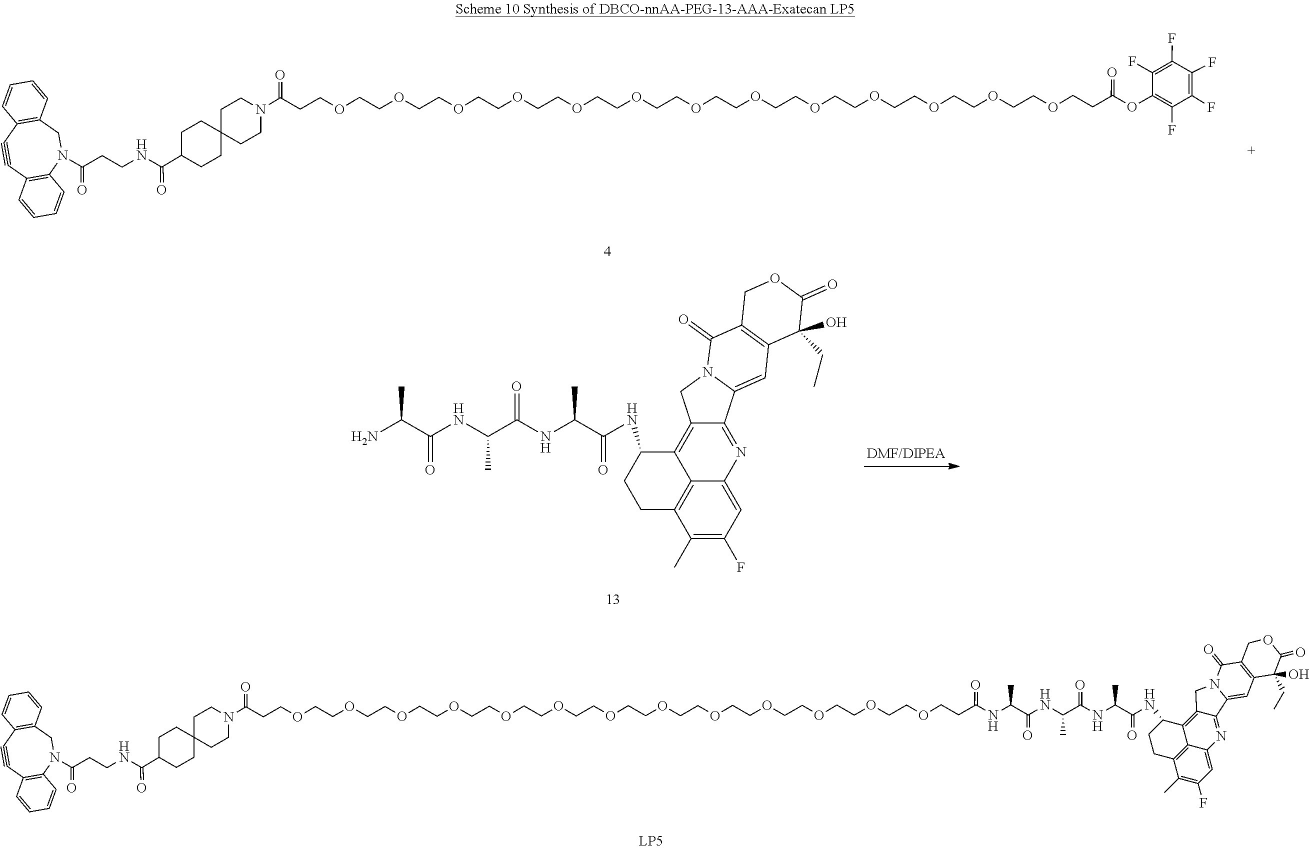 [SMILES: O=C(CCOCCOCCOCCOCCOCCOCCOCCOCCOCCOCCOCCOCCOCCC(=O)N1CCC2(CCC(C(=O)NCCC(=O)N3Cc4ccccc4C#Cc4ccccc43)CC2)CC1)Oc1c(F)c(F)c(F)c(F)c1F ; CC[C@@]1(O)C(=O)OCc2c1cc1n(c2=O)Cc2c-1nc1cc(F)c(C)c3c1c2[C@@H](NC(=O)[C@H](C)NC(=O)[C@H](C)NC(=O)[C@H](C)N)CC3 ; CC[C@@]1(O)C(=O)OCc2c1cc1n(c2=O)Cc2c-1nc1cc(F)c(C)c3c1c2[C@@H](NC(=O)[C@H](C)NC(=O)[C@H](C)NC(=O)[C@H](C)NC(=O)CCOCCOCCOCCOCCOCCOCCOCCOCCOCCOCCOCCOCCOCCC(=O)N1CCC2(CCC(C(=O)NCCC(=O)N4Cc5ccccc5C#Cc5ccccc54)CC2)CC1)CC3]

LP5 was synthesized in an analogous fashion using the same methods as described above. LCMS m/z (ESI⁺): calculated for C₉₂H₁₂₄FN₉O₂₄, 1757.87; found 1759.1 [M+H]⁺.

### Example 14: Para-Azidomethylphenylalanine (PAMF) Incorporation

Non-natural para-azidomethylphenylalanine (pAMF) residues were incorporated in monoclonal antibodies (mAbs) in place of specific residues using XpressCF+® cell-free expression platform (Yin et al., 2017, Sci Rep 7 (1): 3026). Sites were chosen for pAMF incorporation to enable the conjugation of cytotoxin moieties via copper-catalyzed azide-alkyne cycloaddition (CuAAC) or a copper-free conjugation method, e.g., strain-promoted azide-alkyne cycloaddition (SPAAC) through dibenzocyclooctyne (DBCO or DIBO). mAbs were purified through affinity chromatography column followed by ion exchange chromatography columns.

The CFPS reaction mix included 37.5% (v/v) S30 E. coli cell extract, 3 mg/L plasmid DNA, and 2 mM pAMF as well as the 20 natural amino acids, NMPs, polyamines, and small molecules for energy generation (Cai et al. Biotechnol. Prog. 2015, 31, 823-831). M. jannaschii pAMF tRNA synthetase, M. jannaschii amber suppressor tRNA, and T7 RNA polymerase were individually over-expressed in intact E. coli cells and added to the CF reaction as a crude lysate at a final concentration of 1-2% each. Reactions were conducted in a stirred tank bioreactor for 14 hrs at 25° C. at pH 7.0 and 20% DO followed by 3 hrs at pH 8.0 80% DO. mAbs were captured by Protein A affinity chromatography eluted with eluted with 100 mM Glycine at pH 3.2. The neutralized Protein A eluate was concentrated and further polished by preparative SEC preparative SEC (Superdex 200) in phosphate-buffered saline (1×PBS).

### Example 15: General Methods for Conjugation of Compounds to Antibodies

Method #1: Dibenzocylcooctyne (DBCO) compound was dissolved in dimethylsulfoxide (DMSO) to a final concentration of 5 mM. The conjugation was carried out in 1×PBS at antibody concentration of 1-30 mg/mL, DBCO compound to pAMF ratio of 1.5-3, and with 25% DMSO. The reaction mixture was incubated at room temperature for overnight. The conjugation efficiency was measured by LC/MS. Unconjugated DBCO compound was removed by cation exchange. The conjugate was formulated in formulation buffer supplemented with 9% sucrose. The drug to antibody ratio (DAR) was measured by LC/MS.

Method #2: Linker payloads were dissolved in DMSO to a final concentration of 5 mM. The conjugation was carried out in 1×PBS at antibody concentration of 1 mg/mL, drug linker to pAMF ratio of 3, and with 15% of DMSO. The reaction mixture was incubated at room temperature for overnight. The conjugation efficiency was measured by MALDI. Unconjugated drug linker was removed by desalting and formulated in 1×PBS. Purity of the conjugate was measured by Sepax SEC-300.

### Example 16: Differential Scanning Fluorimetry

A protein thermal shift assay was carried out by mixing the protein to be assayed with an environmentally sensitive dye (SYPRO Orange, Life Technologies #S-6650) and monitoring the fluorescence of the mixture in real time as it underwent controlled thermal denaturation. Protein solutions at 1 mg/mL were mixed at a 1-1 volumetric ratio with a solution of SYPRO Orange solution diluted 250-fold in PBS+10% sucrose. 5 μL aliquots of the protein-dye mixture were dispensed in quadruplicate in a 384-well microplate (Bio-Rad #MSP-3852). The plate was sealed with an optically clear sealing film (Bio-Rad #MSB-1001) and placed in a 384-well plate real-time thermocycler (Bio-Rad CFX384 Real Time System). The protein-dye mixture was heated from 25° C. to 95° C., at increments of 0.2° C. per cycle, allowing 3 seconds of equilibration at each temperature before taking a fluorescence measurement. At the end of the experiment, the transition melting temperatures (TM1 and TM2) were determined using the Bio-Rad CFX manager software. TM1 represents the melting temperature of the Fc domain. TM2 represents the melting temperature of the Fab domain.

TABLE 21
DSF Melting temperatures for parent antibody and ADC.

DSF

| | | TM1 | TM2 |
|---|---|---|---|
| Conjugate/mAB # | Description | (° C.) | (° C.) |
| mAb 41 | 2900-A02 F241/F404TAG | 61.6 | 82.6 |
| Conjugate 7 | 2900-A02 F241/F404 LP2 | 72.5 | 81.2 |

### Example 17: Biacore Kinetic Analysis

Anti-Fc polyclonal antibodies were immobilized onto a CM4 chip (Cytiva #BR100534) using amine coupling chemistry (Human Antibody Capture Kit, Cytiva #BR100839). The immobilization steps were carried out at a flow rate of 25 L/min in 1×HBS-EP+buffer (Cytiva #BR100669). The sensor surfaces were activated for 7 min with a mixture of NHS (0.05 M) and EDC (0.2 M). The Anti-Fc antibodies were injected over all 4 flow cells at a concentration of 25 μg/ml in 10 mM sodium acetate, pH 4.5, for 7 min. Ethanolamine (1 M, pH 8.5) was injected for 7 min to block any remaining activated groups. Approximately 6,000 response units (RU) of capture antibody was immobilized on each flow cell. Off-rate and kinetic binding experiments were performed at 25° C. using 1×HBS-EP+buffer. Antibodies or ADCs were injected over the anti-Fc surface at a concentration of 3 μg/mL for 12 seconds at a flow rate of 10 μL/min on flow cells 2, 3 and 4, followed by a buffer wash for 30 seconds at the same flow rate. Kinetic characterization of antibody or ADC samples was carried out with a range of antigen concentrations from about 1-100 nM and 1 injection of 0 nM antigen (for example, 100, 50, 12.5, 3.125, 0.781 and 0 nM). After capturing ligand (antibody) on the anti-Fc surface, the analyte (human TF, Lake Pharma; cynomolgus TF, Sino Bio #90885-C08H; mouse TF, R&D Systems #3178-PA) was bound for 180 seconds, followed by a 420 second dissociation phase at a flow rate of 50 μL/min. Between each ligand capture and analyte binding cycle, regeneration was carried out using 2 injections of 10 mM Glycine pH 2.0 for 30 seconds at 30 μL/min, followed by a 30 second buffer wash step. The data was fit with the Biacore T200 Evaluation software, using a 1-1 Langmuir binding model. KD (affinity, nM) was determined as a ratio of the kinetic rate constants calculated from the fits of the association and dissociation phases.

TABLE 22
Biacore Kinetics for parent antibody and ADCs.

| | | | | | Biacore cynomolgus TF | | |
|---|---|---|---|---|---|---|---|
| | | Biacore human TF kinetics | | | kinetics | | |
| Conjugate/ | | ka | kd | | ka | kd | |
| mAb # | Description | (1/Ms) | (1/s) | KD (M) | (1/Ms) | (1/s) | KD (M) |
| mAb 41 | 2900-A02 | 4.03E+05 | 1.82E−03 | 4.52E−09 | 4.57E+05 | 8.40E−04 | 1.84E−09 |
| | F241/F404TAG | | | | | | |
| Conjugate | 2900-A02 | 5.08E+05 | 1.89E−03 | 3.71E−09 | 7.47E+05 | 8.92E−04 | 1.19E−09 |
| 2 | F241/F404 | | | | | | |
| | LP1 | | | | | | |
| Conjugate | 2900-A02 | 5.08E+05 | 2.09E−03 | 4.11E−09 | 5.82E+05 | 1.03E−03 | 1.77E−09 |
| 7 | F241/F404 | | | | | | |
| | LP2 | | | | | | |

### Example 18: Conjugates 2 and 7 Developability and Thermostability Testing

Thermal stability assessment for Conjugate 2 (HC 2900-A02 F241/F404TAG SEQ ID NO: 3178; LC SEQ ID NO: 3179; conjugated to LP1; aTF_2900-A02_HC_F241/F404TAG_SerOpt pAzMeF+LP1/Trastuzumab LC SerOpt) was evaluated under various stress conditions in one formulation buffer (10 mM citrate, 9% sucrose, pH 6.0). Thermal stability assessment for Conjugate 7 (HC 2900-A02 F241/F404TAG SEQ ID NO: 3178; LC SEQ ID NO: 3179; conjugated to LP2; aTF_2900-A02_HC_F241/F404TAG_SerOpt pAzMeF+LP2; Trastuzumab LC SerOpt) was evaluated under various stress conditions in several different formulation buffers at 10 mg/mL protein concentration. Study 1: Accelerated short term temperature stability was tested for signs of aggregation/degradation at 4° C., 25° C., and 37° C. and evaluated over a 3-week period with sample collected at 3 days, 7 days, 14 days and 21 days. Study 2: Freeze/thaw stability was evaluated over five freeze/thaw cycles. Samples were evaluated by HPLC-SEC. The formulations used in the study include:
Buffer #1:10 mM citrate, 9% sucrose, pH 6.0
Buffer #2:10 mM citrate, 0.01% PS20, 9% sucrose, pH 6.0
Buffer #3:10 mM histidine, 9% sucrose, pH 7.0

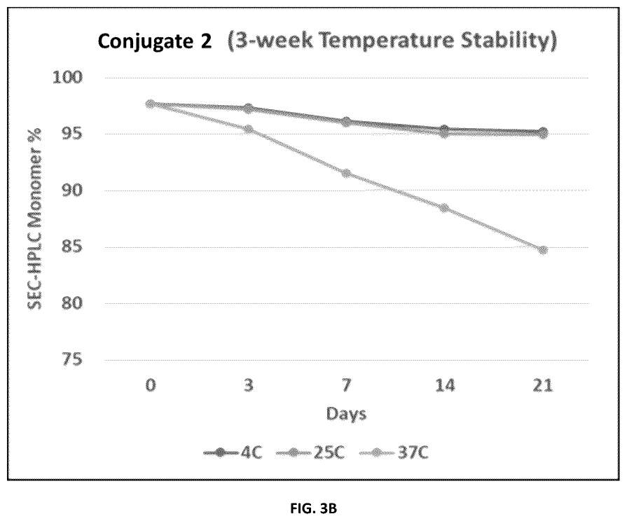

As shown in FIG. 3A, Conjugate 7 at 4° C. and 25° C. did not show significant biophysical changes in any of the formulation buffers, however at 37° C. Conjugate 7 was aggregated which resulted in a decrease of monomer percentage. Buffer #3 which was formulated into histidine did mitigate some monomer % loss at 37° C., while for the other two formulation buffers, there was no impact. Conjugate 2 showed comparable results in FIG. 3B, no significant changes in overall product quality at 4° C. and 25° C., but significantly impacted over the 3-week hold at 37° C.

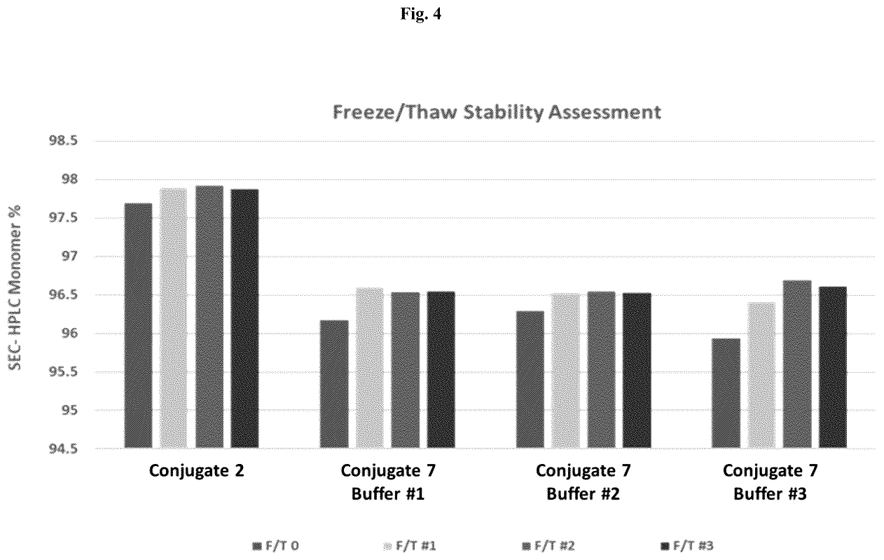

Five freeze/thaw cycles (each aliquot being placed in liquid nitrogen for 10 seconds and then left at ambient temperature until completely thawed) were performed on Conjugate 2 and Conjugate 7. After one, three, and five freeze/thaw cycles, an aliquot from both samples was assayed by HPLC-SEC. HPLC-SEC (FIG. 4) results were consistent across all five freeze/thaw cycles for each sample in all formulation buffers.

### Example 19: Conjugates 2, 3 and 6 Neutrophil Viability Assessment

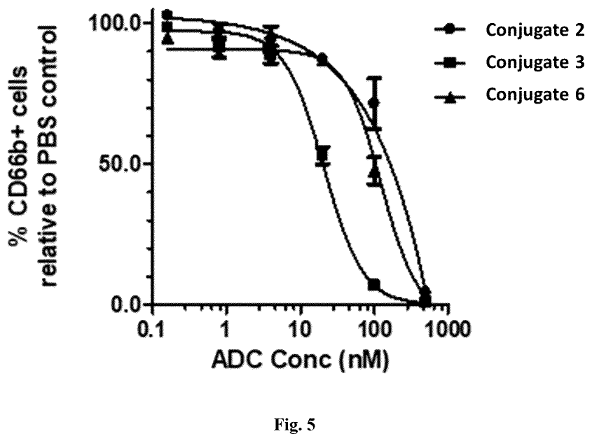

To evaluate the possible toxicity of the ADCs on Neutrophil precursor cells CD34⁺ hematopoietic stem cells were differentiated into Neutrophil precursor cells and treated with the ADCs for 6 days followed by detection with flow cytometry using the CD66b granulocyte marker (Zhao et al., Mol. Cancer Ther. 16, 1866-1876 (2017)). CD34+ cells derived from normal human bone marrow were prepared in media containing X-Vivo-15, rhIL-3 (20 ng/mL), rhIL-6 (20 ng/mL), rhSCF (100 ng/mL) and rhFlt-3L (100 ng/ml). 10,000 cells per well were plated in round-bottom 96-well plates and cultured in a humidified incubator at 37° C., 5% CO2 for 3 days. Cells were then washed twice with X-Vivo medium and incubated in X-Vivo-15 medium supplemented with rhSCF (50 ng/ml), rhFlt-3L (100 ng/ml), rh IL3 (5 ng/ml), rhGM-CSF (5 ng/mL) rhG-CSF (5 ng/mL) for 4 days. The cells were washed again (×2) and incubated in X-Vivo-15 medium supplemented with rhIL-3 (5 ng/ml) and rhG-CSF (30 ng/ml) for another 4 days. On day 11, the cells were washed twice and placed in the final media formulation containing X-Vivo-15 supplemented with just rhG-CSF (30 ng/ml) for the final 6 days. Cells were then treated with ADCs or PBS for a further 6 days. For flow analysis, cells were blocked with 10% FBS and 20 μg/mL human IgG at 4° C. for 10 minutes. Following blocking, the surface markers of the cells were stained with a PE-conjugated anti-CD66b antibody (Biolegend, catalog number 392904) and DAPI (viability) at 4° C. for 20 minutes. The cells were then washed and resuspended in 200 μL per well of PBS+2% FBS, and then analyzed by flow cytometry using a Beckman Coulter cytoFlex cytometer. The viability of Neutrophil precursor cells based on % CD66b+ cell numbers relative to a PBS treated control were tested for Conjugates 2, 3 and 6 (FIG. 5)

### Example 20: In Vitro Cell Killing of Tf Lead Antibodies Conjugated to LP1

Anti-TF antibodies were conjugated to LP1 at Y180/F404 sites to make ADCs of DAR=4 and the cell killing activity of the ADCs was evaluated on TF positive BxPC3, NCI-H292, NCI-H1975, NCI-H441, NCI-H1954, JIMT1, and MDA-MB-468 cells as well as TF negative NCI-H1703 cells.

BxPC3, NCI-H292, NCI-H1975, NCI-H441, NCI-H1954, JIMT1, MDA-MB-468 and NCI-H1703 cells were purchased from ATCC (American Type Culture Collection). All the other cell lines were maintained in DMEM/F12 (1:1), high glucose (Corning) supplemented with 10% heat-inactivated fetal bovine serum (Sigma), 2 mM glutamax (Thermo Scientific), and 1× Penicillin/Streptomycin (Corning). Cytotoxicity effects of the ADC on the cancer cell lines were measured with a cell proliferation assay. A total of 625 cells in a volume of 25 microliters were seeded in a 384-well flat bottom white polystyrene plate the day before the assay. ADC and free drugs were formulated at 2× starting concentration in cell culture medium and filtered through SpinX 0.22 micron cellulose acetate filtered 2 mL centrituge tubes (Corning Costar). Filter sterilized samples were serial diluted (1:3) under sterile conditions and added onto cells in triplicates. Plates were cultured at 37° C. in a CO2 incubator for 120 hours. For cell viability measurement, 30 microliters of Cell Titer-Glo® reagent (Promega Corp, Madison, WI) was added into each well, and plates processed as per product instructions. Relative luminescence was measured on an ENVISION® plate reader (Perkin-Elmer; Waltham, MA). Relative luminescence readings were converted to % viability using untreated cells as controls. Data was fitted with non-linear regression analysis, using log (inhibitor) vs. response, variable slope, 4-parameter fit equation using GraphPad Prism. Data was expressed as % relative cell viability vs. dose of free linker-warhead or ADC in nanomolar with error bars indicating the Standard Deviation (SD) of the triplicates.

The cell killing activity of ADCs for the antibody leads from SRP2799 and SRP2767 on BxPC3 cells were summarized in Table 23. All of the tested ADCs showed similar high potency cell killing on BxPC3 cells.

TABLE 23
Cell killing activity for different TF
antibodies conjugated to LP1 on BxPC3.

| | | | EC50 | Span |
|---|---|---|---|---|
| Conjugate # | Sample | DAR | (nM) | (%) |
| 30 | 2799-A05 Y180/F404-LP1 | 3.9 | 0.025 | 99 |
| 31 | 2799-B03 Y180/F404-LP1 | 3.9 | 0.023 | 99 |
| 32 | 2799-B06 Y180/F404-LP1 | 3.9 | 0.061 | 99 |

The cell killing activity of the ADCs for antibody leads from SRP2900 and SRP2842 were summarized in Table 24. All of the tested ADCs showed potent cell killing on TF positive NCI-H292, NCI-H441, NCI-H1954 and MDA-MB-468, some cell killing on NCI-H1975 and JIMT1 cells, but not much cell killing on TF negative NCI-H1703 cells.

TABLE 24
Cell killing activity of different TF antibodies conjugated to LP1.

| | | NCI-H292 | | NCI-H1975 | | NCI-H441 | | NCI-H703 | |
|---|---|---|---|---|---|---|---|---|---|
| | | (TF++++) | | (TF++) | | (TF+) | | (TF±) | |
| Conjugate | | EC50 | Span | EC50 | Span | EC50 | Span | EC50 | Span |
| # | DAR | (nM) | (%) | (nM) | (%) | (nM) | (%) | (nM) | (%) |
| 1 | 3.8 | 0.06 | 83 | >100 | NC | 0.511 | 53 | >100 | NC |
| 21 | 3.74 | 0.022 | 86 | 0.234 | 66 | 0.116 | 56 | >100 | NC |
| 22 | 3.71 | 0.106 | 82 | >100 | NC | >100 | NC | >100 | NC |
| 23 | 3.71 | 0.048 | 79 | >101 | NC | 1.54 | 42 | >100 | NC |
| 24 | 3.61 | 0.057 | 78 | >102 | NC | 0.803 | 43 | >100 | NC |
| 25 | 3.73 | 0.048 | 78 | >103 | NC | 0.805 | 43 | >100 | NC |
| 26 | 3.71 | 0.177 | 84 | >104 | NC | 0.708 | 55 | >100 | NC |
| 27 | 3.71 | 0.115 | 83 | >105 | NC | 0.738 | 56 | >100 | NC |
| 28 | 3.67 | 0.201 | 81 | >106 | NC | 0.733 | 51 | >100 | NC |
| 29 | 3.73 | 0.066 | 77 | >107 | NC | 0.389 | 45 | >100 | NC |

| | | NCI-H1954 | | JIMT1 | | MDA-MB- | |
|---|---|---|---|---|---|---|---|
| | | (TF+++) | | (TF++) | | 468 (TF++) | |
| Conjugate | | EC50 | Span | EC50 | Span | EC50 | Span |
| # | DAR | (nM) | (%) | (nM) | (%) | (nM) | (%) |
| 1 | 3.8 | 0.133 | 92 | >100 | NC | 0.361 | 88 |
| 21 | 3.74 | 0.022 | 94 | 0.3 | 60 | 0.054 | 87 |
| 22 | 3.71 | 0.966 | 90 | >100 | NC | 3.51 | 93 |
| 23 | 3.71 | 0.186 | 87 | >100 | NC | 0.536 | 84 |
| 24 | 3.61 | 0.165 | 90 | >100 | NC | 0.485 | 85 |
| 25 | 3.73 | 0.149 | 88 | >100 | NC | 0.602 | 81 |
| 26 | 3.71 | 0.172 | 92 | >100 | NC | 0.505 | 85 |
| 27 | 3.71 | 0.151 | 91 | 5.31 | 54 | 0.475 | 86 |
| 28 | 3.67 | 0.222 | 88 | >100 | NC | 0.941 | 86 |
| 29 | 3.73 | 0.175 | 89 | >100 | NC | 1 | 85 |

### Example 21: In Vitro Cell Killing of TF ADCS Conjugated to LP1 at Different Conjugation Sites and DARS

Anti-TF antibody 2900-A02 was conjugated to LP1 at different conjugation sites with different DARs. The cell killing activity of the ADCs was evaluated on TF positive NCI-H292, NCI-H1975, HCC1954, as well as TF negative NCI-H1703 cells.

The cell killing EC50 the ADCs were summarized in Table 25. TF ADCs with different DARs showed similar high potency cell killing on TF positive NCI-H292 and HCC1954, relatively lower cell killing activity on TF low NCI-H1975, and not much cell killing on TF negative NCI-H1703 cells.

TABLE 25
Cell killing activity of TF ADCs conjugated to LP1 at different sites and DARs.

| | | NCI-H292 | | HCC1954 | | NCI- | | NCI-H1703 | |
|---|---|---|---|---|---|---|---|---|---|
| | | (TF++++) | | (TF+++) | | H1975(TF++) | | (TF±) | |
| | | EC50 | Span | EC50 | Span | EC50 | Span | EC50 | Span |
| Conjugate # | DAR | (nM) | (%) | (nM) | (%) | (nM) | (%) | (nM) | (%) |
| 2 | 3.98 | 0.056 | 86 | 0.094 | 93 | 9.1 | 82 | >100 | NC |
| 4 | 5.91 | 0.038 | 89 | 0.083 | 94 | 6.2 | 91 | >100 | NC |
| 1 | 3.96 | 0.064 | 85 | 0.15 | 93 | 13 | 84 | >100 | NC |
| 3 | 5.97 | 0.042 | 87 | 0.13 | 94 | 6.5 | 79 | >100 | NC |

To evaluate if the cell killing observed on these cells are specific to TF expression on the positive cells, unconjugated antibodies were included at 0.5 μM to compete with the cell killing of the ADCs.

The cell killing activity of the ADCs in the presence and absence of the unconjugated antibodies were summarized in Table 26. TF ADCs with different DARs showed similar high potency cell killing on TF positive NCI-H292 and HCC1954, relatively lower cell killing activity on TF low NCI-H1975. The cell killing activity of the ADCs on TF positive cells was greatly reduced in the presence of 0.5 μM of un-conjugated antibodies, which indicated that the cell killing of TF ADCs was depended on the binding of antibodies to the TF on the cell surface. Very weak cell killing activity was observed for TF ADCs on TF negative NCI-H1703 cells and this weak cell killing was not affected by the presence of un-conjugated antibodies, which indicated that the weak killing on TF negative cells might be not specific to TF expression.

TABLE 26
Competition cell killing activity of TF ADCs conjugated
to LP1 at different sites and DARs.

| | | NCI-H292 | | HCC1954 | | NCI- | | NCI-H1703 | |
|---|---|---|---|---|---|---|---|---|---|
| | | (TF++++) | | (TF+++) | | H1975(TF++) | | (TF±) | |
| | | EC50 | Span | EC50 | Span | EC50 | Span | EC50 | Span |
| Conjugate # | DAR | (nM) | (%) | (nM) | (%) | (nM) | (%) | (nM) | (%) |
| 1 | 3.96 | 0.055 | 85 | 0.064 | 95 | ~25 | 62 | >100 | NC |
| 3 | 5.97 | 0.048 | 82 | 0.073 | 93 | ~7 | 68 | >100 | NC |
| 5 | 7.5 | 0.045 | 87 | 0.08 | 91 | ~4.4 | 72 | >100 | NC |
| 6 | 7.94 | 0.044 | 86 | 0.085 | 93 | ~5 | 71 | >100 | NC |
| 1 + 0.5 uM mAb | 3.96 | >100 | NC | >100 | NC | >100 | NC | >100 | NC |
| 36 | | | | | | | | | |
| 3 + 0.5 uM mAb | 5.97 | >100 | NC | >100 | NC | >100 | NC | >100 | NC |
| 39 | | | | | | | | | |
| 5 + 0.5 uM mAb | 7.5 | >100 | NC | >100 | NC | >100 | NC | >100 | NC |
| 38 | | | | | | | | | |
| 6 + 0.5 uM mAb | 7.94 | >100 | NC | >100 | NC | >100 | NC | >100 | NC |
| 40 | | | | | | | | | |

### Example 22: In Vitro Cell Killing of TF ADCS Conjugated to Different Exatecan Linker Payloads

Anti-TF antibody 2900-A02 and 2842-B01 were conjugated to exatecan payload with different linkers and the cell killing activity of the ADCs was evaluated on TF positive NCI-H292, NCI-H1975, HCC1954, as well as TF negative NCI-H1703 cells.

2900-A02 conjugated to exatecan linkers (LP2, LP3, LP4, LP5) showed various cell killing activity on TF positive NCI-H292 and HCC1954, not much cell killing on TF relative low NCI-H1975 and TF negative NCI-H1703 cells. 2842-B01 conjugated to LP3, LP4 and LP5 showed potent cell killing activity on NCI-H292 cells, but no cell killing on other cells tested. The cell killing activity of the ADCs were summarized in Table 27.

TABLE 27
Cell killing activity of TF ADCs conjugated to Exatecan linker payloads.

| | | NCI-H292 | | HCC1954 | | NCI- | | NCI-H1703 | |
|---|---|---|---|---|---|---|---|---|---|
| | | (TF++++) | | (TF+++) | | H1975(TF++) | | (TF±) | |
| Conjugate | | EC50 | Span | EC50 | Span | EC50 | Span | EC50 | Span |
| or mAb # | DAR | (nM) | (%) | (nM) | (%) | (nM) | (%) | (nM) | (%) |
| 9 | 3.63 | 0.3 | 74 | >100 | NC | >100 | NC | >100 | NC |
| 10 | 3.72 | 0.049 | 91 | 0.28 | 40 | >100 | NC | >100 | NC |
| 11 | 3.74 | 0.086 | 90 | 0.49 | 38 | >100 | NC | >100 | NC |
| 12 | 3.81 | 0.16 | 88 | 2.1 | 39 | >100 | NC | >100 | NC |
| 18 | 3.99 | 0.23 | 90 | >100 | NC | >100 | NC | >100 | NC |
| 19 | 3.99 | 0.41 | 89 | >100 | NC | >100 | NC | >100 | NC |
| 20 | 3.99 | 0.67 | 86 | >100 | NC | >100 | NC | >100 | NC |

The TF antibody 2900-A02 was also conjugated to exatecan linker payload LP2 at DAR=4 (Conjugate 7) and DAR=8 (Conjugate 8) and the cell killing activities were compared to LP1 ADC at DAR=4 (Conjugate 2) on TF positive MDA-MB-231, BxPC3, NCI-H292, A431, NCI-H1975 and NCI-H441 cells, as well as TF negative NCI-H1703 cells.

The hemiasterlin ADC at DAR4 (Conjugate 2) showed potent cell killing on TF positive MDA-MB-231, BxPC3, NCI-H292 and A431. Some cell killing was observed on NCI-H441 cells, but not much cell killing on NCI-H1975 and NCI-H1703 cells. The exatecan ADCs showed potent cell killing on BxPC3, NCI-H292 and A431 cells, but not much cell killing on other cells tested. In general, the exatecan ADCs showed lower cell killing activity on TF positive cells compared to Hemiasterlin ADC at DAR=4. Exatecan ADC at DAR8 showed slightly better cell killing than exatecan ADC at DAR4 on TF positive cells. The cell killing activities were summarized in Table 28.

TABLE 28
Cell killing activity of TF ADCs conjugated to LP2 at DAR4 and DAR8.

| | Conjugate #2 | | Conjugate #7 | | Conjugate #8 | |
|---|---|---|---|---|---|---|
| | EC₅₀ | | EC₅₀ | | EC₅₀ | |
| Cell Line Tested | (nM) | Span (%) | (nM) | Span (%) | (nM) | Span (%) |
| MDA-MB-231 | 0.159 | 66 | 6.363 | 33 | 3.808 | 35 |
| BxPC3 | 0.64 | 96 | 0.469 | 58 | 0.735 | 61 |
| NCI-H292 | 0.639 | 82 | 0.509 | 72 | 0.326 | 75 |
| A431 | 0.711 | 97 | 0.697 | 82 | 0.589 | 80 |
| NCI-H1975 | >100 | NC | >100 | NC | >100 | NC |
| NCI-H441 | 1 | 40 | >100 | NC | >100 | NC |
| NCI-H1703 | >100 | NC | >100 | NC | >100 | NC |

### Example 23: In Vitro Cell Binding of TF Antibodies on Primary Keratinocytes

Since TF is expressed in human skin, anti-TF antibody 2900-A02 and 2842-B01-Hc3/Lc2 were tested in cell binding assay on primary adult human keratinocyte cells purchased from Thermo Fisher Scientific. Keratinocytes were cultured in defined keratinocyte culture medium (EpiLife Medium, Thermo Fisher Scientific) supplemented with growth supplement S7 (S0175 from Thermo Fisher Scientific) and Penicillin/Streptomycin on plates coated with keratinocyte coating matrix (Thermo Fisher Scientific).

To evaluate antibody binding activity of the anti-TF antibodies on the adult primary keratinocytes, a total of 100,000 keratinocytes per well were incubated on ice with serial dilutions of anti-TF antibodies in FACS buffer (PBS buffer supplemented with 1% bovine serum albumin, 0.05% sodium azide) for 60 minutes. Cells were washed twice with ice-cold FACS buffer and then incubated with 5 microgram/mL Alexa 647 labeled donkey anti-human Fc antibody (Jackson ImmunoResearch, West Grove, PA) on ice for another 60 minutes. Unstained cells and cells stained with secondary antibodies alone were used as controls. Samples were then washed twice using FACS buffer and analyzed using an Attune flow cytometer (Thermo Fisher). Geometric mean fluorescence intensities were fitted using non-linear regression analysis with one site specific binding equation on GraphPad Prism.

The TF antibody 2900-A02 (mAb 36) bound to human primary keratinocytes with high affinity, while the TF antibody lead 2842-B01-HC3/LC2 (mAb 37) showed reduced affinity to human primary keratinocytes. The cell binding activity of the antibodies on human primary keratinocytes was summarized in Table 29.

TABLE 29
TF antibodies cell binding activity
on primary human keratinocytes.

| | | Keratinocyte Binding | |
|---|---|---|---|
| mAb# | Name | Bmax | Kd (nM) |
| 36 | 2900-A02 Y180/F404 | 164743 | 3.1 |
| 37 | 2842-B01-HC3/LC2 Y180/F404 | 151831 | 24 |

### Example 24: In Vitro Cell Killing of TF ADCS on Primary Keratinocytes

Anti-TF antibody 2900-A02 ADCs conjugated to LP1 at different conjugation sites with different DARs, as well as TF antibody 2842-B01-HC3/Lc2 conjugated to LP1 at DAR4, were tested in cell killing assay on primary adult human keratinocyte cells.

To evaluate the cytotoxicity effects of the ADC on the adult primary keratinocytes, a total of 2000 keratinocytes in a volume of 50 microliters were seeded in a 96-well flat bottom cell culture plate coated with keratinocyte coating matrix (Thermo Fisher Scientific) the day before the actual assay starts. ADCs were formulated at 2× starting concentration in cell culture medium and filtered through SpinX 0.22 micron cellulose acetate filtered 2 mL centrifuge tubes (Corning Costar). Filter sterilized samples were serial diluted (1:3) under sterile conditions and added onto cells in triplicates. Plates were cultured at 37° C. in a CO2 incubator for 120 hours. For cell viability measurement, 10 microliters of WST-8 reagent (VWR) were added into each well and plates were incubated at 37° C. in a CO2 incubator for 4 hours. The absorbance at 450 nm was measured using a microplate reader (M5, Molecular Devices) and readings were converted to % viability using untreated cells as controls. Data was fitted with non-linear regression analysis, using log (inhibitor) vs. response, variable slope, 4-parameter fit equation using GraphPad Prism. Data was expressed as % relative cell viability vs. dose of free linker-warhead or ADC in nanomolar with error bars indicating the Standard Deviation (SD) of the triplicates.

ADCs for the TF antibody 2900-A02 showed similar potent cell killing on human primary keratinocytes, while the ADC for the lower affinity TF antibody 2842-B01-HC3/Lc2 showed reduced cell killing activity compared to 2900-A2 ADCs. The cell killing activity of the ADCs on human primary keratinocytes were summarized in Table 30.

TABLE 30
TF ADCs cell killing and binding activity
on primary human keratinocytes.

| | | | Keratinocyte Killing | |
|---|---|---|---|---|
| Conjugate | | | EC50 | Span |
| or mAb# | Name | DAR | (nM) | (%) |
| 36 | 2900-A02 Y180/F404 | — | >100 | NC |
| 1 | 2900-A02 Y180/F404-LP1 | 4 | 0.0055 | 73 |
| 3 | 2900-A02 Y180/F404/k42-LP1 | 6 | 0.0091 | 74 |
| 5 | 2900-A02 Y180/F404/ | 7.5 | 0.0062 | 72 |
| | k42/E161-LP1 | | | |
| 6 | 2900-A02 Y180/F241/ | 7.9 | 0.0048 | 74 |
| | F404/k42-LP1 | | | |
| 37 | 2842-B01-HC3/LC2 | — | >100 | NC |
| | Y180/F404 | | | |
| 26 | 2842-B01-HC3/LC2 | 3.9 | 0.038 | 73 |
| | Y180/F404-LP1 | | | |
| 7 | 2900-A02_HC_F241/F404-LP2 | 3.96 | 14.2 | 60 |
| 35 | 2900-A02_Y180/F241/ | 7.83 | 0.781 | 74 |
| | Y391/F404-LP2 | | | |

### Example 25: In Vitro Cell Killing of TF ADCS on Primary Human Corneal Epithelial Cells

Anti-TF antibody 2900-A02 ADCs conjugated to LP2 at different DARs (Conjugate 7 and conjugate 35) were tested in cell killing assay on primary human corneal epithelial cells to evaluate potential eye toxicity.

Primary human corneal epithelial cells (HCECs) were purchased from ATCC and maintained in corneal epithelial cell growth medium (corneal epithelial cell basal medium supplied with corneal epithelial cell growth kit from ATCC that contains apo-transferrin, epinephrine, Extract P, hydrocortisone hemisuccinate, L-Glutamine, recombinant human insulin and CE Growth Factor). To evaluate the cytotoxicity effects of the ADCs on HCECs, a total of 625 cells in a volume of 25 microliters of corneal epithelial cell growth medium were seeded in a 384-well white bottom cell culture plate the day before the actual assay starts. ADCs were formulated at 2× starting concentration in cell culture medium and filtered through SpinX 0.22 micron cellulose acetate filtered 2 mL centrifuge tubes (Corning Costar). Filter sterilized samples were serial diluted (1:3) under sterile conditions and 25 microliters were added onto cells in quadroplicates. Plates were cultured at 37° C. in a CO2 incubator for 7 days. For cell viability measurement, 10 microliters of PrestoBlue reagent were added into each well and plates were incubated at 37° C. in a CO2 incubator for 3 hours. The fluorescence was measured using a microplate reader (M5, Molecular Devices) and readings were converted to % viability using untreated cells as controls. Data was fitted with non-linear regression analysis, using log (inhibitor) vs. response, variable slope, 4-parameter fit equation using GraphPad Prism. Data was expressed as % relative cell viability vs. dose of ADC in nanomolar with error bars indicating the Standard Deviation (SD) of the quadroplicates.

ADCs for the TF antibody 2900-A02 showed weak cell killing on human primary corneal epithelial, while DAR8 ADC conjugate 35 showed slightly more activity compared to DAR4 ADC (conjugate 7). The cell killing activity of the ADCs were summarized in Table 31.

TABLE 31
TF ADCs cell killing on primary human corneal epithelial cells.

| HCEC | Conjugate 7 | | Conjugate 35 | |
|---|---|---|---|---|
| Lot# | EC50 (nM) | Span (%) | EC50 (nM) | Span (%) |
| 8120122 | 0.33 | 63.6 | 0.11 | 62.5 |
| 1001654 | 0.79 | 52 | 0.29 | 65 |
| 70036737 | 0.41 | 60.1 | 0.25 | 67.5 |

### Example 26: DAR Stability

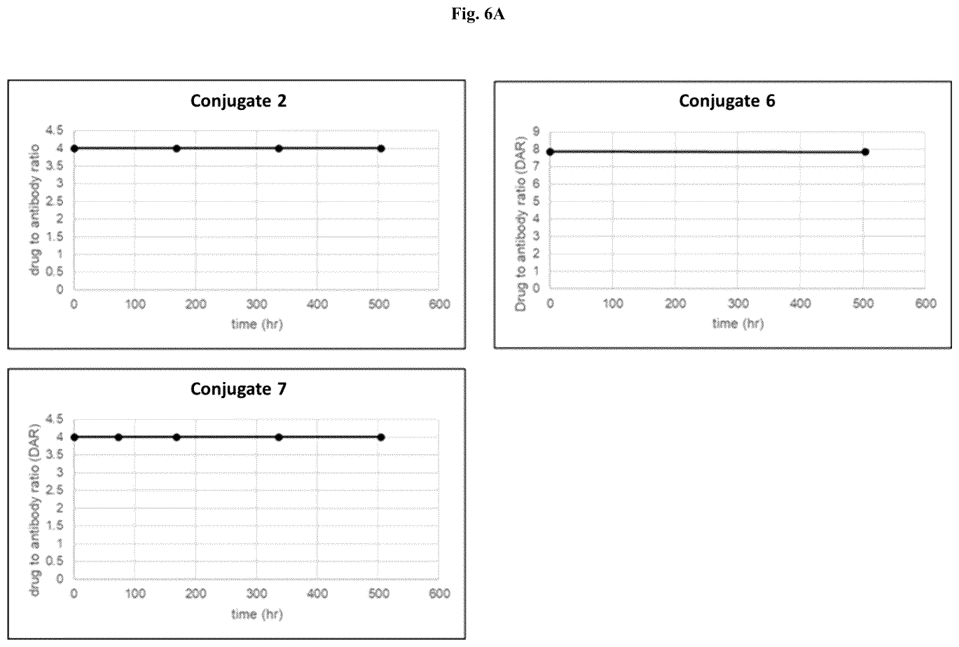

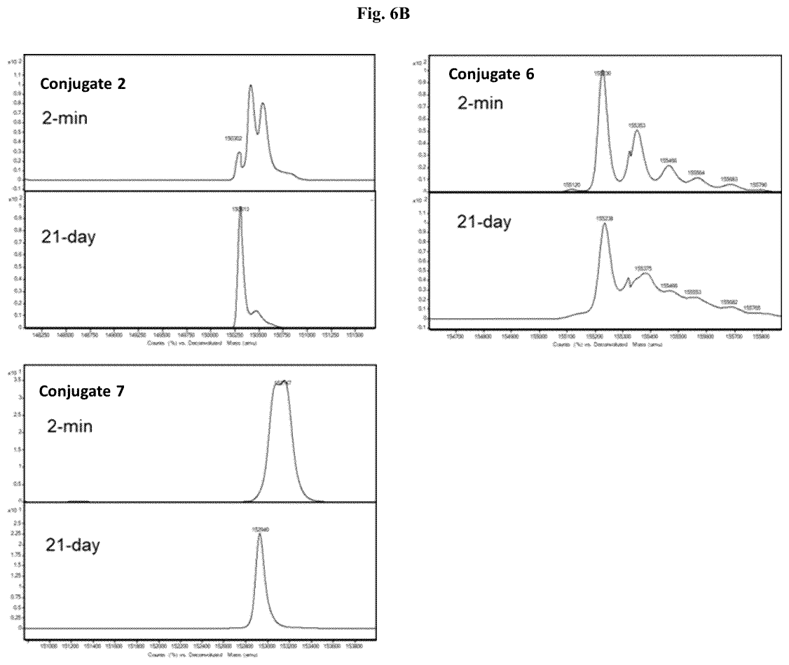

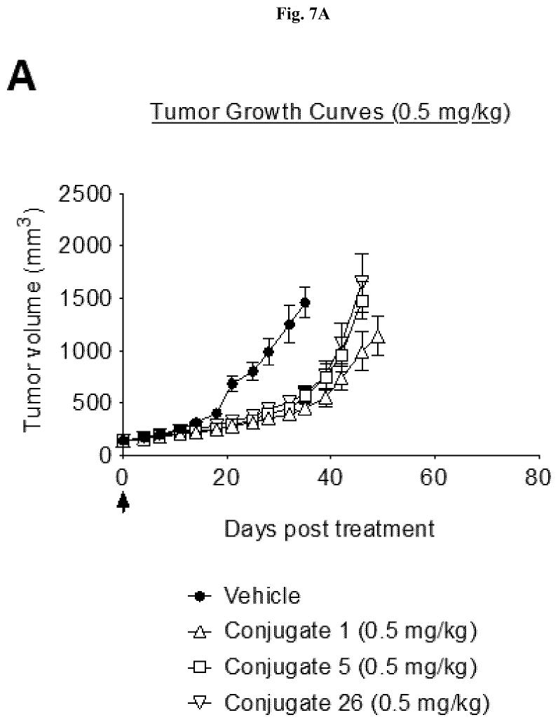

In vivo linker payload stability of Conjugates 2, 6 and 7 was investigated in rats or cynomolgus monkeys. Briefly, Conjugates 2, 6 and 7 were injected through IV. Plasma samples were collected from 2-5 animals at 15 min, 3 days, 7 days, 14 days and 21 days after dosing and were kept frozen until ready to be analyzed. aTF ADC was pulled down from plasma using anti-hFc and then injected on to Agilent QToF for intact mass analysis. DAR was calculated based on deconvoluted peak area. The three ADCs tested were all stable over 21 days (FIG. 6A). Comparison of 15-min sample and day 21 sample demonstrated that there was no degradation products observed over 21 days (FIG. 6B). Multiple peaks detected in 15-min samples were due to HC C-terminal lysine clipping that was common from in vivo samples.

### Example 27: In Vivo Efficacy in the HCC1954 Breast Cancer Xenograft Model

The activity of different TF-targeted ADCs was examined in the HCC1954 breast cancer xenograft model. Briefly, BALB/c nude mice were implanted subcutaneously with 5×10⁶ HCC1954 tumor cells in the right flank and randomized and enrolled into the study 13 days post implant, with tumor sizes around 140 mm³. Tumor-bearing mice were administered a single dose of the test articles i.v. at 0.5 mg/kg and 5 mg/kg. All treatments were well tolerated, and mice exhibited normal body weight gain throughout the course of the study.

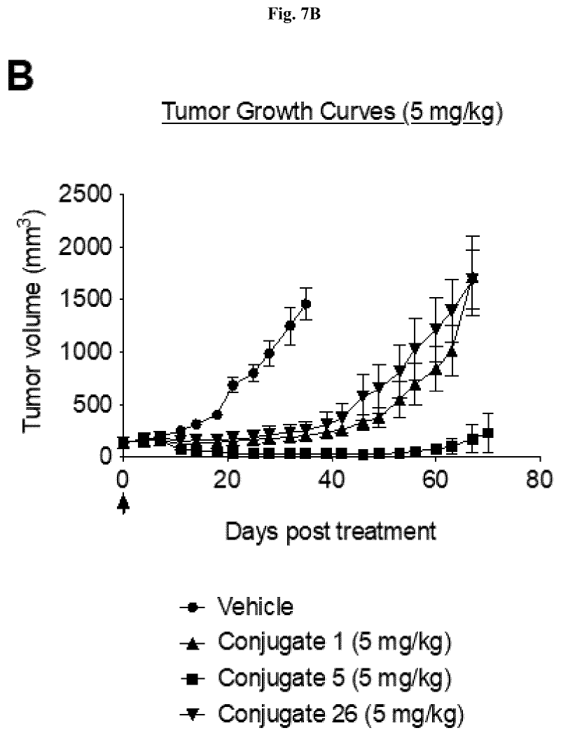

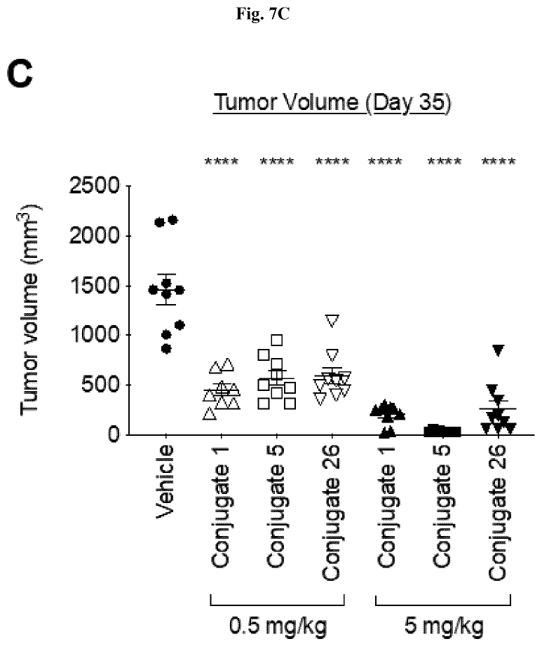

FIGS. 7A and 7B illustrate the effects of the different test articles on HCC1954 tumor growth up until the end of the study at day 70 post treatment. Analysis of tumor sizes on day 35, when the mean of vehicle-treated tumors reached the study endpoint (˜1,500 mm³), revealed that all test articles demonstrated significant anti-tumor activity, with efficacy ranging from approximately 60% to 110% TGI (FIG. 7C). At 0.5 mg/kg, Conjugate 1, Conjugate 5, and Conjugate 26 exhibited moderate and roughly equivalent tumor growth suppression (73%, 62%, and 60% TGI, respectively). At 5 mg/kg, Conjugate 1, Conjugate 5, and Conjugate 26 all demonstrated robust tumor growth inhibition (94%, 110%, and 90% TGI, respectively), with Conjugate 5 exhibiting the greatest potency. Notably, Conjugate 5 was the only test article to achieve sustained tumor regression, with no evidence of tumor outgrowth until day 53 post treatment (FIG. 7B).

### Example 28: In Vivo Efficacy in the H1975 Lung Cancer Xenograft Model

The activity of different TF-targeted ADCs was examined in the H1975 lung cancer xenograft model. Briefly, SCID Beige mice were implanted subcutaneously with 5×10⁶ H1975 tumor cells in the right flank and randomized and enrolled into the study 10 days post implant, with tumor sizes around 130 mm³. Tumor-bearing mice were administered a single dose of the test articles i.v. at 1 mg/kg or 2 mg/kg. All treatments were well tolerated, and mice exhibited normal body weight gain throughout the course of the study.

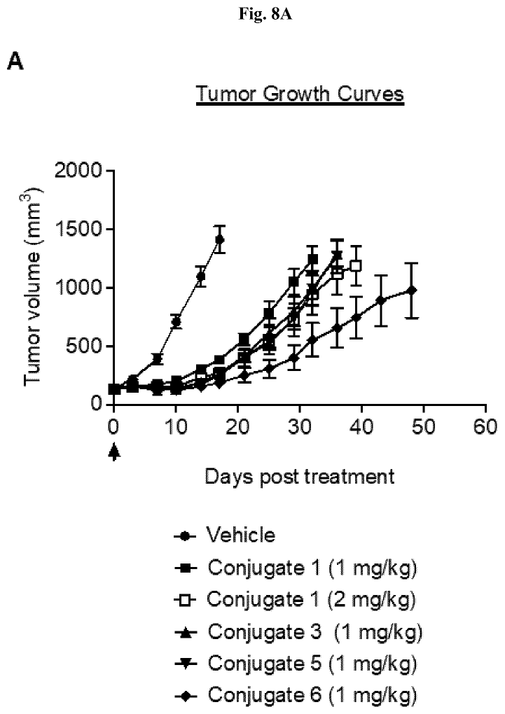

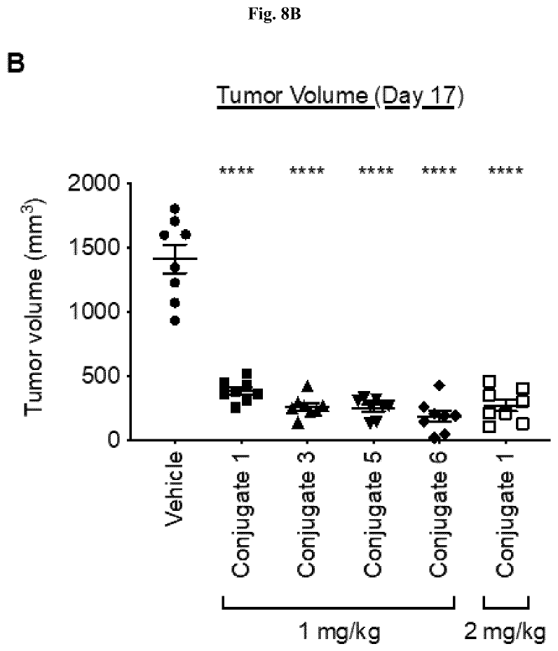

FIG. 8A illustrates the effects of the different test articles on H1975 tumor growth up until the end of the study at day 48 post treatment. Analysis of tumor sizes on day 17, when the mean of vehicle-treated tumors reached the study endpoint (˜1,500 mm³), revealed that all test articles demonstrated significant anti-tumor activity, with efficacy ranging from approximately 80% to 96% TGI (FIG. 8B). At 1 mg/kg, Conjugate 1, Conjugate 3, Conjugate 5, and Conjugate 6 all showed potent and roughly equivalent tumor growth suppression (80%, 90%, 91%, and 96% TGI, respectively).

### Example 29: In Vivo Efficacy in the H1975 Lung Cancer Xenograft Model

The activity of different TF-targeted ADCs was examined in the H1975 lung cancer xenograft model. Briefly, SCID Beige mice were implanted subcutaneously with 5×10⁶ H1975 tumor cells in the right flank and randomized and enrolled into the study 10 days post implant, with tumor sizes around 120 mm³. Tumor-bearing mice were administered a single dose of the test articles i.v. at doses ranging from 0.5 mg/kg to 2 mg/kg. All treatments were well tolerated, and mice exhibited normal body weight gain throughout the course of the study.

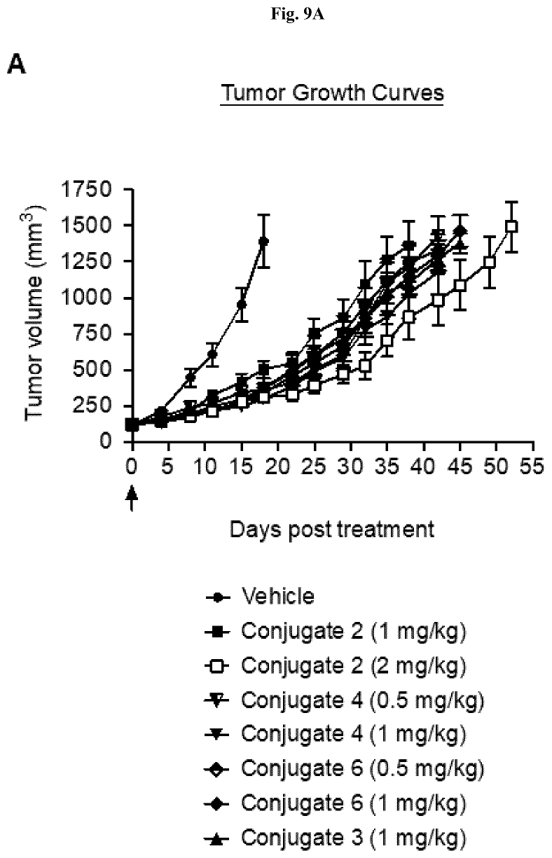

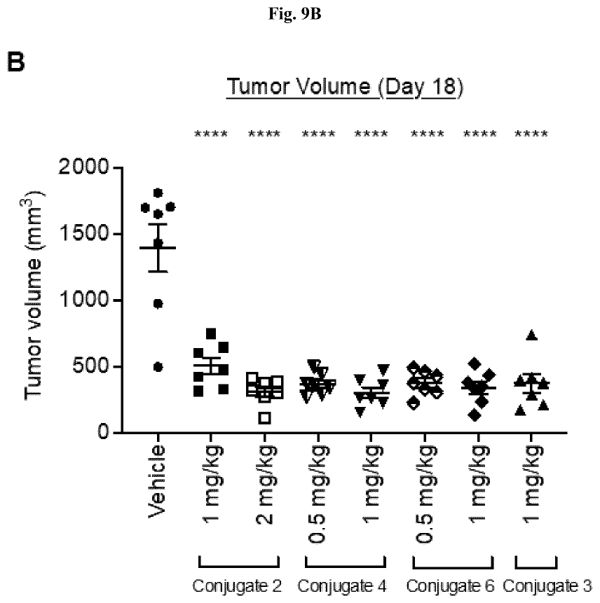

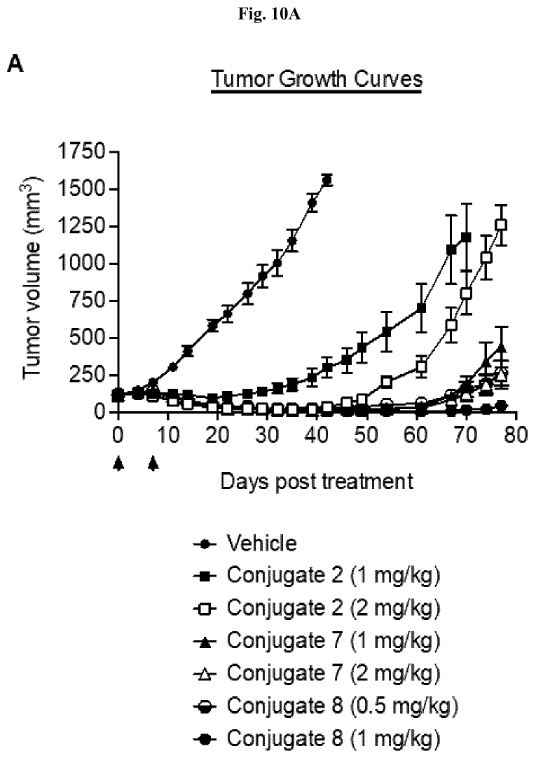

FIG. 9A illustrates the effects of the different test articles on H1975 tumor growth up until the end of the study at day 52 post treatment. Analysis of tumor sizes on day 18, when the mean of vehicle-treated tumors reached the study endpoint (˜1,400 mm³), revealed that all test articles demonstrated significant anti-tumor activity, with efficacy ranging from approximately 70% to 86% TGI (FIG. 9B). At 1 mg/kg, Conjugate 2, Conjugate 4, Conjugate 6, and Conjugate 3 all showed potent and roughly equivalent tumor growth suppression (70%, 80%, 86%, and 83% TGI, respectively). Conjugate 4 and Conjugate 6 dosed at 0.5 mg/kg and Conjugate 2 dosed at 2 mg/kg also exhibited similar levels of tumor growth inhibition (81%, 80%, and 85% TGI, respectively).

### Example 30: In Vivo Efficacy in the MDA-MB-231 TNBC Xenograft Model

The activity of different TF-targeted ADCs was examined in the MDA-MB-231 TNBC xenograft model. Briefly, SCID Beige mice were implanted subcutaneously with 5×10⁶ MDA-MB-231 tumor cells in the mammary fat pad and randomized and enrolled into the study with an average tumor volume of 110-120 mm³. Tumor-bearing mice were administered two weekly doses (qwx2) of the test articles i.v. at doses ranging from 0.25 mg/kg to 2 mg/kg. All treatments were well tolerated, and mice exhibited normal body weight gain throughout the course of the study.

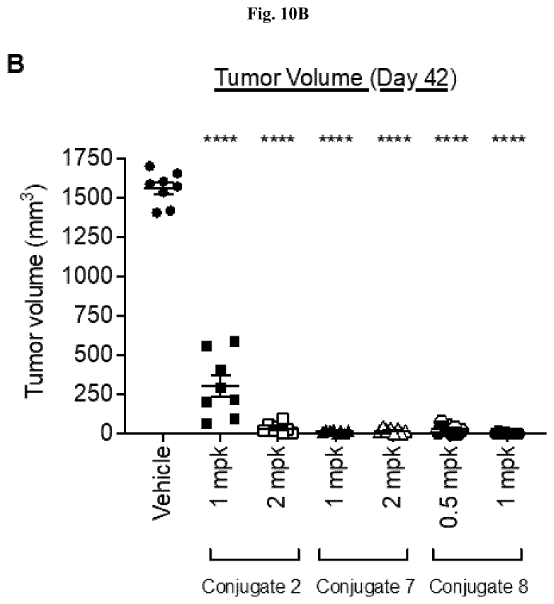

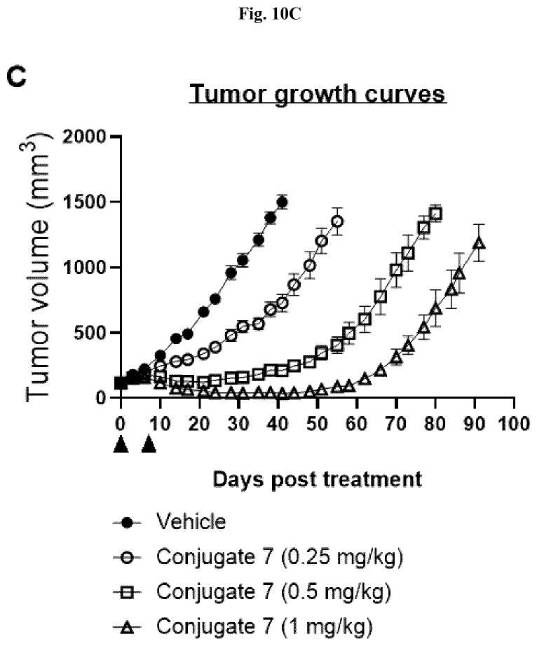

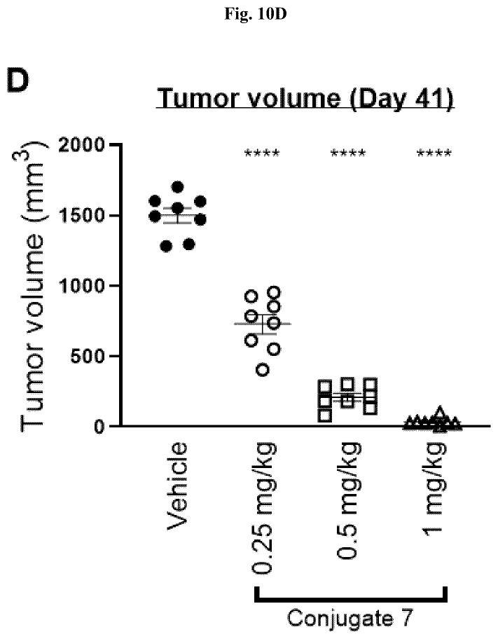

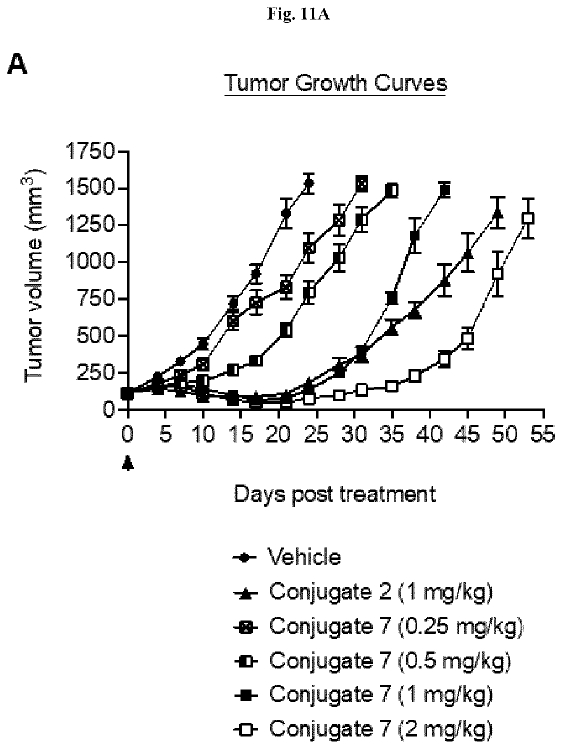

FIG. 10A illustrates the effects of the different test articles on MDA-MB-231 tumor growth up until the end of the study at day 77 post treatment. Analysis of tumor sizes on day 42, when the mean of vehicle-treated tumors reached the study endpoint (˜1,500 mm³), revealed that all test articles demonstrated significant anti-tumor activity, with efficacy ranging from approximately 87% to 108% TGI (FIG. 10B). At 1 mg/kg, Conjugate 2, Conjugate 7, and Conjugate 8 all exhibited potent anti-tumor efficacy (87%, 107%, and 108% TGI, respectively), achieving prolonged tumor stasis or tumor regression following treatment. Dose-dependent activity was observed for Conjugate 2, with 1 mg/kg and 2 mg/kg treatments achieving 87% and 106% TGI, respectively, while Conjugate 7 and Conjugate 8 did not appear to exhibit clear dose-dependent activity at these doses (Conjugate 7 1 mg/kg and 2 mg/kg both achieved 107% TGI, and Conjugate 8 0.5 mg/kg and 1 mg/kg achieved 106% and 108% TGI, respectively). Notably, Conjugate 7 and Conjugate 8 demonstrated similar activity when compared to each other but improved activity compared to Conjugate 2. Furthermore, only Conjugate 7 and Conjugate 8 were able to achieve complete responses. FIG. 10C illustrates the effects of the different test articles on MDA-MB-231 tumor growth up until the end of the study at day 91 post treatment. Analysis of tumor sizes on day 41, when the mean of vehicle-treated tumors reached the study endpoint (˜1,500 mm³), revealed that Conjugate 7 demonstrated significant anti-tumor activity, with efficacy ranging from approximately 56% to 106% TGI (FIG. 10D). Dose-dependent activity was observed for Conjugate 7, with 0.5 mg/kg and 1 mg/kg treatments achieving potent anti-tumor efficacy (93% and 106% TGI, respectively) and prolonged tumor stasis or tumor regression following treatment.

### Example 31: In Vivo Efficacy in the H1975 Lung Cancer Xenograft Model

The activity of different TF-targeted ADCs was examined in the H1975 lung cancer xenograft model. Briefly, SCID Beige mice were implanted subcutaneously with 5×10⁶ H1975 tumor cells in the right flank and randomized and enrolled into the study with tumor sizes around 110 mm³. Tumor-bearing mice were administered a single dose of the test articles i.v. at doses ranging from 0.25 mg/kg to 2 mg/kg. All treatments were well tolerated, and mice exhibited normal body weight gain throughout the course of the study.

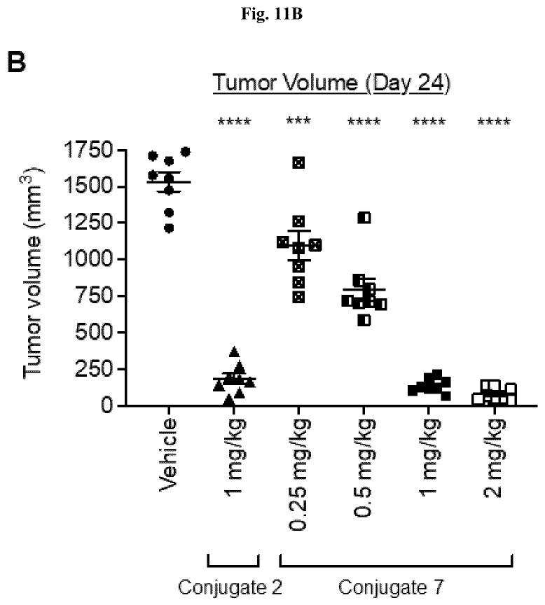

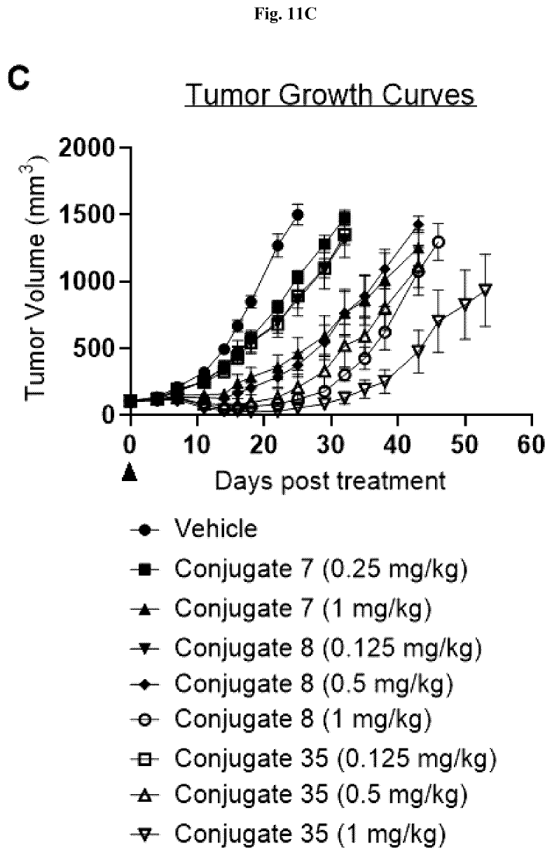

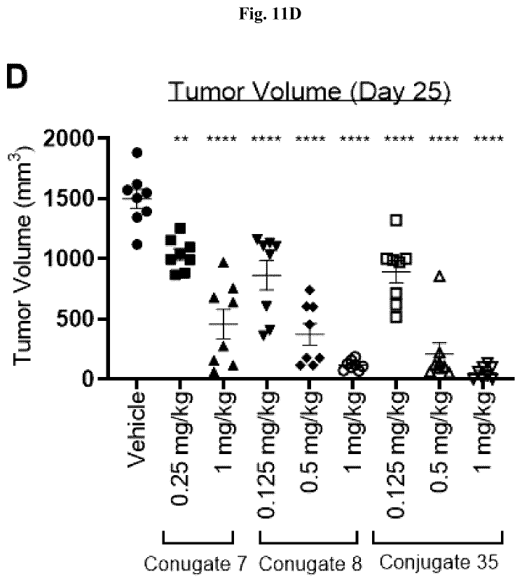

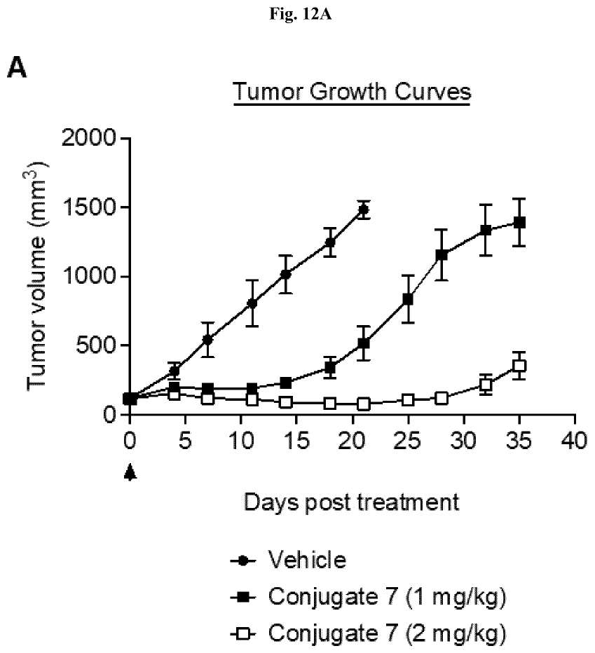

FIG. 11A illustrates the effects of the two test articles on H1975 tumor growth up until the end of the study at day 53 post treatment. Analysis of tumor sizes on day 24, when the mean of vehicle-treated tumors reached the study endpoint (˜1,500 mm³), revealed that both test articles demonstrated significant anti-tumor activity, with efficacy ranging from approximately 31% to 102% TGI (FIG. 11B). Conjugate 7 demonstrated robust dose-dependent activity, with a single 0.25, 0.5, 1, and 2 mg/kg dose achieving 31%, 52%, 98%, and 102% TGI, respectively. Conjugate 7 and Conjugate 2 dosed at 1 mg/kg exhibited similar efficacies at day 24 post treatment (both achieved 98% TGI). FIG. 11C illustrates the effects of the different test articles on H1975 tumor growth up until the end of the study at day 53 post treatment. Analysis of tumor sizes on day 25, when the mean of vehicle-treated tumors reached the study endpoint (˜1,500 mm³), revealed that all test articles demonstrated dose dependent anti-tumor activity, with efficacy ranging from approximately 33% to 104% TGI (FIG. 11D). Conjugate 8 at 1 mg/kg, and Conjugate 35 at 0.5 mg/kg and 1 mg/kg exhibited potent anti-tumor efficacy (99%, 99%, and 104% TGI, respectively), achieving prolonged tumor stasis or tumor regression following treatment.

### Example 32: In Vivo Efficacy in the Detroit562 Pharyngeal Xenograft Model

The activity of different TF-targeted ADCs was examined in the Detroit562 pharyngeal cancer xenograft model. Briefly, SCID Beige mice were implanted subcutaneously with 5×10⁶ Detroit562 tumor cells in the hind flank and randomized and enrolled into the study with an average tumor volume of 115 to 120 mm³. Tumor-bearing mice were administered a single dose of Conjugate 7 i.v. at 1 mg/kg or 2 mg/kg. All treatments were well tolerated, with mice exhibiting normal body weight gain throughout the course of the study.

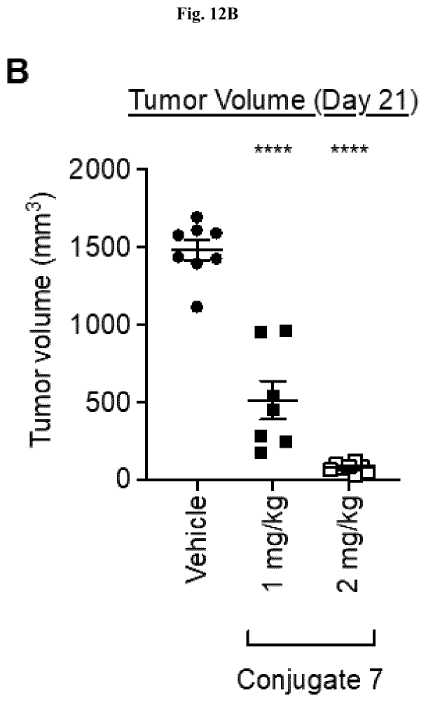

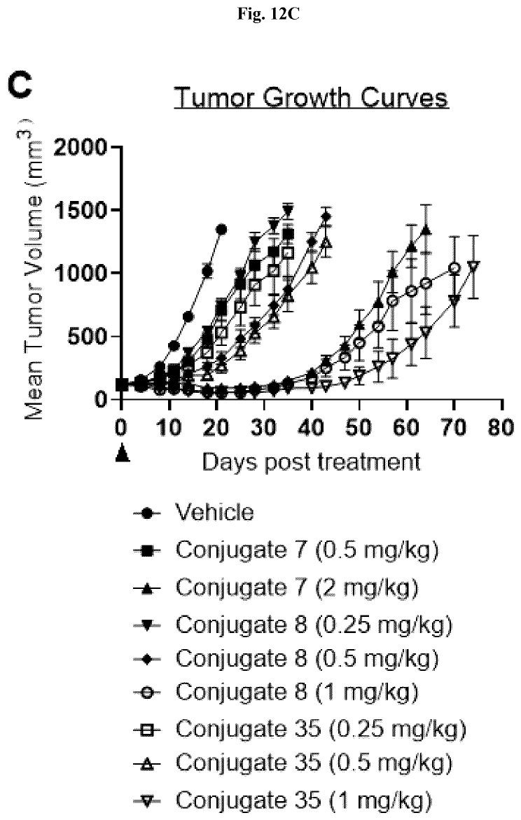

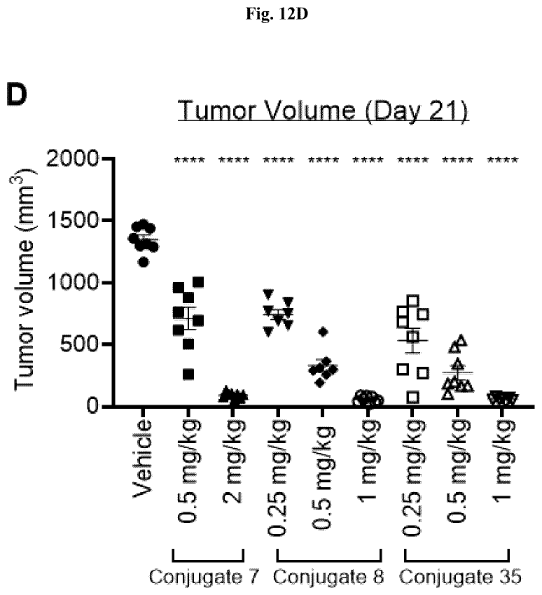

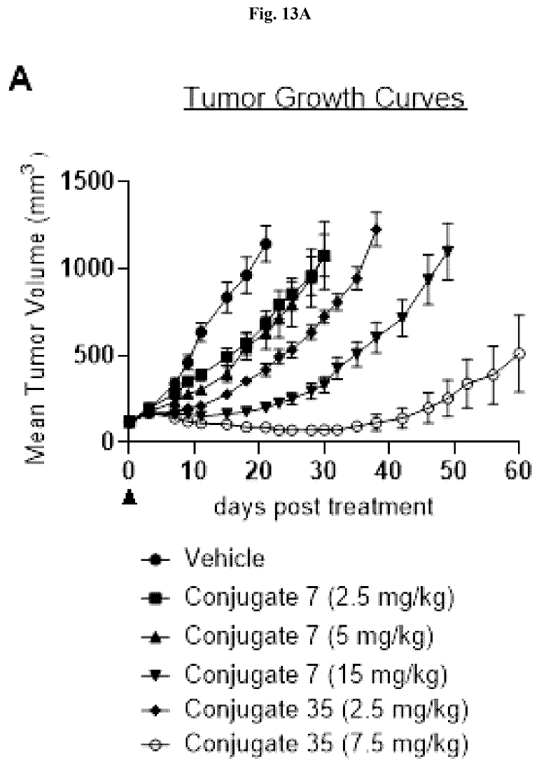

FIG. 12A illustrates the effects of Conjugate 7 on Detroit562 tumor growth up until the end of the study at day 35 post treatment. Analysis of tumor sizes on day 21, when the mean of vehicle-treated tumors reached the study endpoint (˜1,500 mm³), revealed that Conjugate 7 demonstrated significant dose-dependent anti-tumor activity. A single dose of Conjugate 7 at 1 mg/kg and 2 mg/kg achieved 72% and 103% TGI, respectively (FIG. 12B). While Conjugate 7 dosed at 1 mg/kg only slowed tumor growth, Conjugate 7 dosed at 2 mg/kg achieved sustained tumor regression, with no evidence of tumor outgrowth until day 25 post treatment (FIG. 12A). FIG. 12C illustrates the effects of the different test articles on Detroit562 tumor growth up until the end of the study at day 53 post treatment. Analysis of tumor sizes on day 21, when the mean of vehicle-treated tumors reached the study endpoint (˜1,300 mm³), revealed that all test articles demonstrated dose dependent anti-tumor activity, with efficacy ranging from approximately 49% to 105% TGI (FIG. 12D). Conjugate 7 at 2 mg/kg and Conjugate 8 and Conjugate 35 at 1 mg/kg exhibited potent anti-tumor efficacy (102%, 105%, and 105% TGI, respectively), achieving prolonged tumor stasis or tumor regression following treatment.

### Example 33: In Vivo Efficacy in the HCT-116 Colon Xenograft Model

The activity of different TF-targeted ADCs was examined in the HCT-116 colon cancer xenograft model. Briefly, athymic nude mice were implanted subcutaneously with 4×10⁶ HCT-116 tumor cells in the hind flank and randomized and enrolled into the study with tumor sizes around 150 mm³. Tumor-bearing mice were administered a single dose of the test articles i.v. at doses ranging from 2.5 mg/kg to 7.5 mg/kg. All treatments were well tolerated, with mice exhibiting normal body weight gain throughout the course of the study.

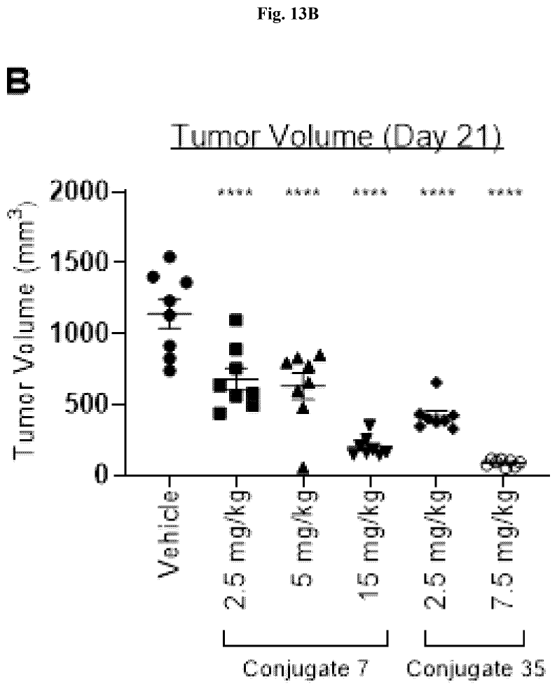

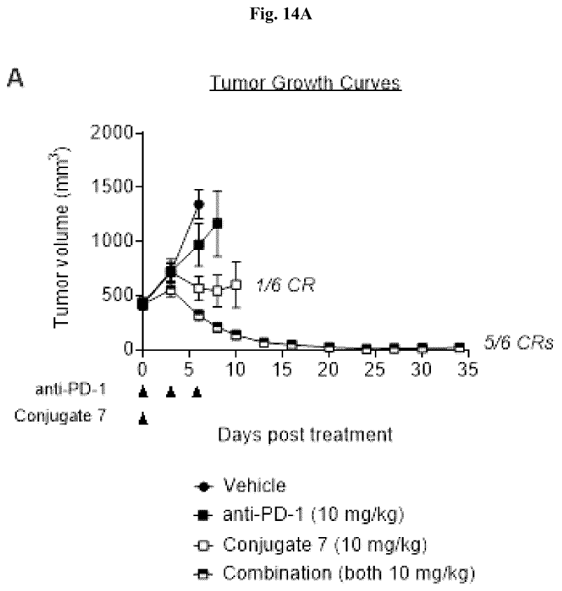

FIG. 13A illustrates the effects of the different test articles on HCT-116 tumor growth up until the end of the study at day 60 post treatment. Analysis of tumor sizes on day 21, when the mean of vehicle-treated tumors reached the study endpoint (˜1,200 mm³), revealed that both test articles demonstrated dose dependent anti-tumor activity, with efficacy ranging from approximately 45% to 103% TGI (FIG. 13B). Conjugate 35 at 7.5 mg/kg exhibited potent anti-tumor efficacy achieving prolonged tumor regression following treatment.

### Example 34: Combination with Immune Checkpoint Blockcade

The activity of TF-targeted conjugates as a single agent or in combination with anti-PD-1 was assessed in a syngeneic model using a mouse colorectal cancer line engineered to express human TF, MC38-hTF. Briefly, C57BL/6 mice were implanted subcutaneously with 1×10⁶ MC38-hTF tumor cells in the hind flank and randomized and enrolled with tumor sizes around 400 mm³. Tumor-bearing mice were administered a single dose of TF-targeted conjugates i.v. at the indicated doses with or without anti-PD-1 (q3d-q4dx3-4) i.p. at 10 mg/kg. All treatments were well tolerated, with mice exhibiting normal body weight gain throughout the course of the study.

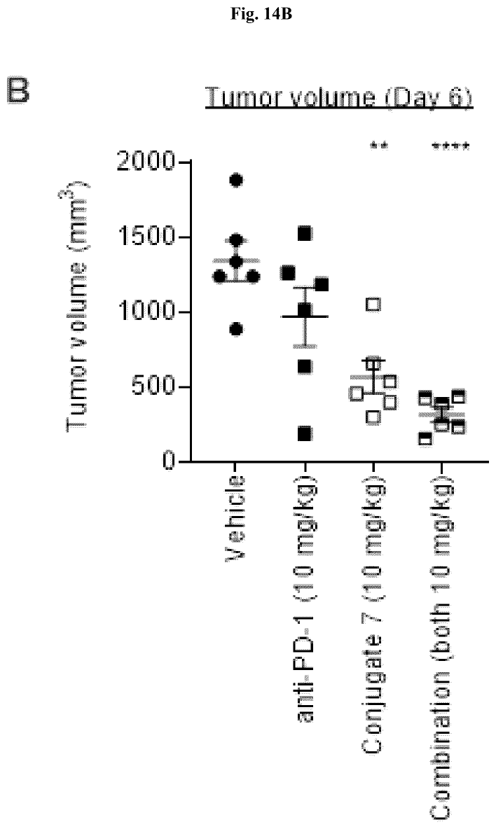

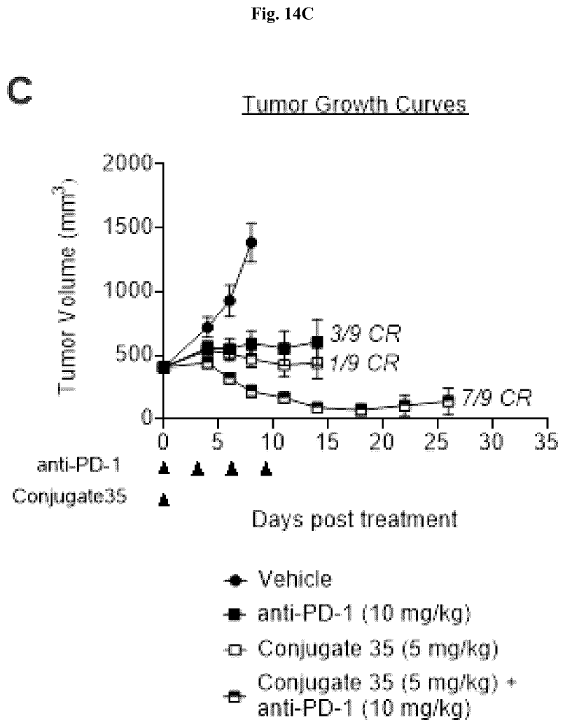

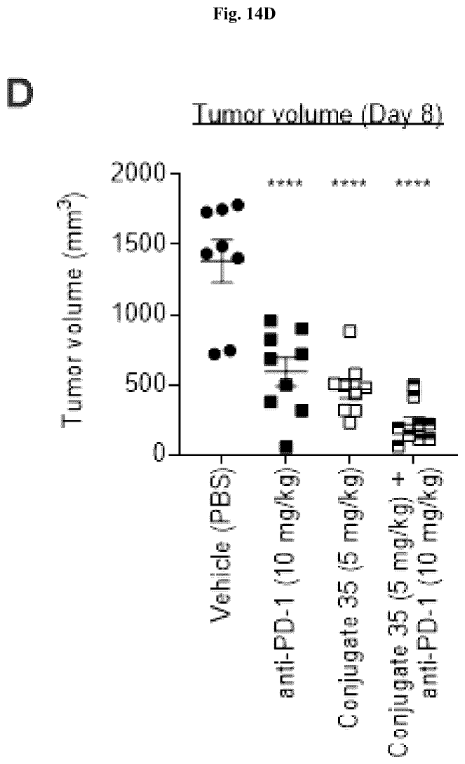

FIG. 14A illustrates the effects of Conjugate 7 monotherapy, anti-PD-1 monotherapy, and combination treatment on MC38-hTF tumor growth up until the end of the study at day 34 post treatment. Analysis of tumor sizes on day 6, when the mean of vehicle-treated tumors reached the study endpoint (>1,300 mm³), revealed that Conjugate 7 and the combination treatment demonstrated significant anti-tumor activity, and treatment with anti-PD-1 monotherapy was not found to be significantly different from the vehicle-treated group (FIG. 14B). Notably, combination treatment demonstrated lasting tumor regression and achieved a greater number of complete responses (CRs) (⅙ and ⅚ mice in the Conjugate 7 monotherapy and combination treatment groups, respectively) (FIG. 14A). FIG. 14C illustrates the effects of Conjugate 35 monotherapy, anti-PD-1 monotherapy, and combination treatment on MC38-hTF tumor growth up until the end of the study at day 26 post treatment. Analysis of tumor sizes on day 8, when the mean of vehicle-treated tumors reached the study endpoint (>1,300 mm³), revealed that Conjugate 35, anti-PD-1 monotherapy, and the combination treatment demonstrated significant anti-tumor activity (FIG. 14D). Notably, combination treatment demonstrated lasting tumor regression and achieved a greater number of complete responses (CRs) (⅙, 3/9, and 5/9 mice in the Conjugate 35, anti-PD-1 monotherapy, and combination treatment groups, respectively) (FIG. 14C).

### Example 35: Efficacy of TF-Targeted Conjugates in PDX Models

The activity of different TF-targeted conjugates was examined in NSCLC, head and neck cancer, and esophageal patient-derived xenograft (PDX) models. Briefly, PDX tumors, spanning varying levels of TF expression, are passaged in athymic nude FoxInu mice before being processed into tumor fragments for subcutaneous implantation into the hind flank of mice for study initiation. When tumors reach a volume of around 150-300 mm³, the mice are randomized and enrolled into the study. Tumor-bearing mice are administered a single dose of the test articles i.v. at the indicated dose levels.

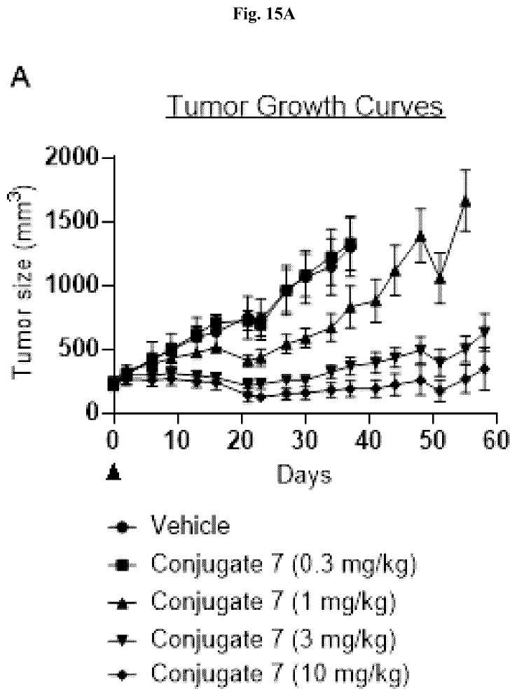

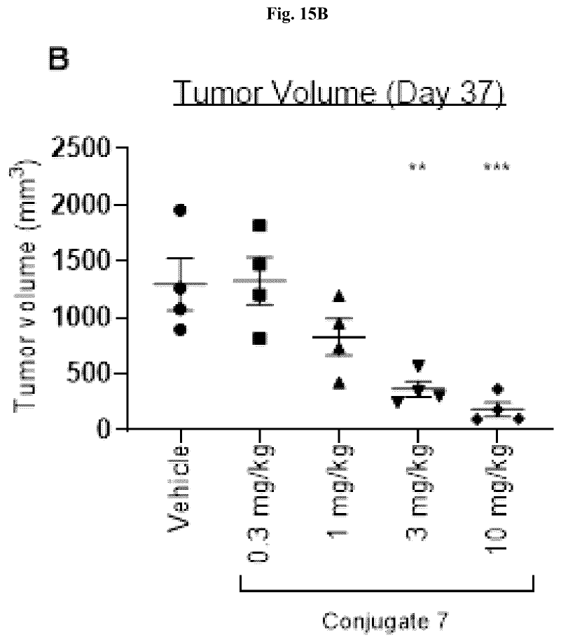

FIG. 15A illustrates the effects of Conjugate 7 on tumor growth of a NSCLC PDX model at doses ranging from 0.1 mg/kg to 10 mg/kg model up until the end of the study at day 58 post treatment. Analysis of tumor sizes on day 37 revealed that Conjugate 7 demonstrated dose-dependent anti-tumor activity. A single dose of Conjugate 7 at 3 mg/kg and 10 mg/kg achieved significant tumor growth inhibition compared to the vehicle group. (FIG. 15B).

### Example 36: Safety Evaluation of Tissue Factor-Targeted Therapies

It is suspected that Tissue Factor-targeted therapies could present the potential for systemic toxicity (myelosuppression) and on-target toxicity potential because TF is widely expressed in the skin, eye, and gastrointestinal tissues with key risks being bleeding, skin toxicity, and eye toxicity.

A study was designed in Non-Human Primate (NHP) using Tissue Factor antibody drug conjugates. TF were characterized by TMDD with a conjugate of the present disclosure administered between 5 and 100 mg/kg q3w ×2 for lower DAR variants or at 25-50 mg/kg q3w x2 for higher DAR variants. Pharmacokinetics was assessed for significant exposure with TMDD.

### Example 37: Preclinical Testing

An antibody conjugate of the present disclosure was assessed for tolerance in NHP up to 100 mg/kg with dose-dependent exposures achieved. Safety was monitored for whether MTD/HNSTD was not reached at 100 mg/kg. Myelosuppression was monitored based on changes in neutrophiles, platelets and WBCs. Liver enzymes were observed and safety concerns were also monitored by histopathology.

The data demonstrates that a potent and specific antibody that does not interfere with the coagulation cascade can provide a potent and clinically validated exatecan payload with broad activity in solid tumors, incorporating a β-glucuronidase linker for improved stability and hydrophilicity. The antibody conjugate can be optimized for the linker-payload for stability. The antibody conjugate demonstrates potent, dose dependent anti-tumor activity in models of lung, breast, head and neck cancer, as well as checkpoint refractory tumors. The antibody conjugate was also well-tolerated in NHPs without reaching MTD at 100 mg/kg. Any toxicities observed were mild to minimal and ADCs were generally well-tolerated with minimal body weight loss. Thus, it is believed, without being bound to theory that the antibody conjugate has a widened therapeutic index.

### Example 38: Sequences

Table 32 provides sequences referred to herein. Note an N-terminal methionine has been introduced to accommodate translation in a bacterially-derived expression system for each distinct protein chain for the illustrative antibodies (e.g. V_{H} and VL sequences). * indicates the position of TAG codons for non-natural amino acids (in certain embodiments of the sequences, the non-natural amino acid is pAzMeF). It should be understood that sequences recited and claimed in this disclosure can be with or without an N-terminal methionine, with or without an N-terminal methionine-alanine, or with or without an N-terminal MSKNK.

TABLE 32
Sequences.

| SEQ ID | | | | |
|---|---|---|---|---|
| NO: | Molecule | Region | Scheme | Sequence |
| 1 | Human Tissue | | | METPAWPRVPRPETAVARTLLLGWVFAQ |
| | Factor | | | VAGASGTTNTVAAYNLTWKSTNFKTILE |
| | | | | WEPKPVNQVYTVQISTKSGDWKSKCFYT |
| | | | | TDTECDLTDEIVKDVKQTYLARVFSYPA |
| | | | | GNVESTGSAGEPLYENSPEFTPYLETNL |
| | | | | GQPTIQSFEQVGTKVNVTVEDERTLVRR |
| | | | | NNTFLSLRDVFGKDLIYTLYYWKSSSSG |
| | | | | KKTAKTNTNEFLIDVDKGENYCFSVQAV |
| | | | | IPSRTVNRKSTDSPVECMGQEKGEFREI |
| | | | | FYIIGAVVFVVIILVIILAISLHKCRKA |
| | | | | GVGQSWKENSPLNVS |
| 2 | Cynomolgus | | | METPAWPRVPRPETAVARTLLLGWVFAQVAG |
| | Tissue Factor | | | ASGTTNTVAAYNLTWKSTNFKTILEWEPKPI |
| | | | | NQVYTVQISTKSGDWKSKCFYTADTECDLTD |
| | | | | EIVKDVKQTYLARVESYPAGHVESTGSTEEP |
| | | | | PYENSPEFTPYLETNLGQPTIQSFEQVGTKV |
| | | | | NVTVQDEWTLVRRNDTFLSLRDVFGKDLIYT |
| | | | | LYYWKSSSSGKKTAKTNTNEFLIDVDKGENY |
| | | | | CFSVQAVIPSRRTANRKSTDSPVECMGHEKG |
| | | | | ESREIFYIIGAVVFVVIILVIILAISLHKCK |
| | | | | KARVGRSWKENSPLNVA |
| 105 | SRP2799-A05 | CDR-H1 | Chothia | GFNISSY |
| 114 | SRP2799-B03 | CDR-H1 | Chothia | GFNISDY |
| 117 | SRP2799-B06 | CDR-H1 | Chothia | GFNISDY |
| 129 | SRP2900-A01 | CDR-H1 | Chothia | GFTISSY |
| 130 | SRP2900-A02 | CDR-H1 | Chothia | GFTISGY |
| 131 | SRP2900-A03 | CDR-H1 | Chothia | GFTIPSY |
| 132 | SRP2900-A04 | CDR-H1 | Chothia | GFTISGY |
| 133 | SRP2900-A05 | CDR-H1 | Chothia | GFTIPDY |
| 134 | SRP2900-A06 | CDR-H1 | Chothia | GFTISSY |
| 135 | SRP2900-A07 | CDR-H1 | Chothia | GFTISSY |
| 136 | SRP2900-A08 | CDR-H1 | Chothia | GFTLTGY |
| 137 | SRP2900-A09 | CDR-H1 | Chothia | GFTIADY |
| 138 | SRP2900-A10 | CDR-H1 | Chothia | GFTISSY |
| 139 | SRP2900-A11 | CDR-H1 | Chothia | GFTITDY |
| 140 | SRP2900-B01 | CDR-H1 | Chothia | GFTISGY |
| 141 | SRP2900-B02 | CDR-H1 | Chothia | GFTISDY |
| 142 | SRP2900-B03 | CDR-H1 | Chothia | GFTIADY |
| 143 | SRP2900-B04 | CDR-H1 | Chothia | GFTISEY |
| 144 | SRP2900-B05 | CDR-H1 | Chothia | GFTISGY |
| 145 | SRP2900-B06 | CDR-H1 | Chothia | GFTIPSY |
| 146 | SRP2900-B07 | CDR-H1 | Chothia | GFTIADY |
| 147 | SRP2900-B08 | CDR-H1 | Chothia | GFTIPSY |
| 148 | SRP2900-B09 | CDR-H1 | Chothia | GFTISQY |
| 149 | SRP2900-B10 | CDR-H1 | Chothia | GFTIPAY |
| 150 | SRP2900-B11 | CDR-H1 | Chothia | GFTIPAY |
| 151 | SRP2900-C01 | CDR-H1 | Chothia | GFTIGSY |
| 152 | SRP2900-C02 | CDR-H1 | Chothia | GFTISSY |
| 153 | SRP2900-C03 | CDR-H1 | Chothia | GFTISGY |
| 154 | SRP2900-C04 | CDR-H1 | Chothia | GFTIVSY |
| 155 | SRP2900-C05 | CDR-H1 | Chothia | GFTISGY |
| 156 | SRP2900-C06 | CDR-H1 | Chothia | GFTISGY |
| 157 | SRP2900-C07 | CDR-H1 | Chothia | GFTISGY |
| 158 | SRP2900-C08 | CDR-H1 | Chothia | GFTIPAY |
| 159 | SRP2900-C09 | CDR-H1 | Chothia | GFTISDY |
| 160 | SRP2900-C10 | CDR-H1 | Chothia | GFTISDY |
| 161 | SRP2900-C11 | CDR-H1 | Chothia | GFTISQY |
| 162 | SRP2900-D01 | CDR-H1 | Chothia | GFTIHEY |
| 163 | SRP2900-D02 | CDR-H1 | Chothia | GFTISDY |
| 164 | SRP2900-D03 | CDR-H1 | Chothia | GFTISQY |
| 165 | SRP2900-D04 | CDR-H1 | Chothia | GFTISDY |
| 166 | SRP2900-D05 | CDR-H1 | Chothia | GFAIANY |
| 167 | SRP2900-D06 | CDR-H1 | Chothia | GFTISDY |
| 168 | SRP2900-D07 | CDR-H1 | Chothia | GFTISDY |
| 169 | SRP2900-D08 | CDR-H1 | Chothia | GFTISDY |
| 170 | SRP2900-D09 | CDR-H1 | Chothia | GFTISGY |
| 171 | SRP2900-D10 | CDR-H1 | Chothia | GFTISDY |
| 172 | SRP2900-D11 | CDR-H1 | Chothia | GFTISDY |
| 173 | SRP2900-E01 | CDR-H1 | Chothia | GFTISDY |
| 174 | SRP2900-E02 | CDR-H1 | Chothia | GFTISDY |
| 175 | SRP2900-E03 | CDR-H1 | Chothia | GFTISDY |
| 176 | SRP2900-E04 | CDR-H1 | Chothia | GFTIPDY |
| 177 | SRP2900-E05 | CDR-H1 | Chothia | GFTISGY |
| 178 | SRP2900-E06 | CDR-H1 | Chothia | GFTISGY |
| 179 | SRP2900-E07 | CDR-H1 | Chothia | GFTISQY |
| 180 | SRP2900-E08 | CDR-H1 | Chothia | GFTITDY |
| 181 | SRP2900-E09 | CDR-H1 | Chothia | GFTISQY |
| 182 | SRP2900-E10 | CDR-H1 | Chothia | GFTISDY |
| 183 | SRP2900-E11 | CDR-H1 | Chothia | GFTISYY |
| 184 | SRP2900-F01 | CDR-H1 | Chothia | GFTISDY |
| 185 | SRP2900-F02 | CDR-H1 | Chothia | GFTIGVY |
| 186 | SRP2900-F03 | CDR-H1 | Chothia | GFTISDY |
| 187 | SRP2900-F04 | CDR-H1 | Chothia | GFTISDY |
| 188 | SRP2900-F05 | CDR-H1 | Chothia | GFTINDY |
| 189 | SRP2900-F06 | CDR-H1 | Chothia | GFTISIY |
| 190 | SRP2900-F07 | CDR-H1 | Chothia | GFTISDY |
| 191 | SRP2900-F08 | CDR-H1 | Chothia | GFTIAYY |
| 192 | SRP2900-F09 | CDR-H1 | Chothia | GFTIHDY |
| 193 | SRP2900-F10 | CDR-H1 | Chothia | GFTISDY |
| 194 | SRP2900-F11 | CDR-H1 | Chothia | GFTISGY |
| 195 | SRP2900-G01 | CDR-H1 | Chothia | GFTIFDY |
| 196 | SRP2900-G02 | CDR-H1 | Chothia | GFTISYY |
| 197 | SRP2900-G03 | CDR-H1 | Chothia | GFTINNY |
| 198 | SRP2900-G04 | CDR-H1 | Chothia | GFTTRDY |
| 199 | SRP2900-G05 | CDR-H1 | Chothia | GFTIAYY |
| 200 | SRP2900-G06 | CDR-H1 | Chothia | GFTISDY |
| 201 | SRP2900-G07 | CDR-H1 | Chothia | GFTISDY |
| 202 | SRP2900-G08 | CDR-H1 | Chothia | GFTIGGY |
| 203 | SRP2900-G09 | CDR-H1 | Chothia | GFTISDY |
| 204 | SRP2900-G10 | CDR-H1 | Chothia | GFTIPDY |
| 205 | SRP2900-G11 | CDR-H1 | Chothia | GFTIYDY |
| 206 | SRP2900-H01 | CDR-H1 | Chothia | GFTIFDY |
| 207 | SRP2900-H02 | CDR-H1 | Chothia | GFTIFDY |
| 208 | SRP2900-H03 | CDR-H1 | Chothia | GFTIYDY |
| 209 | SRP2900-H04 | CDR-H1 | Chothia | GFTISHY |
| 210 | SRP2900-H05 | CDR-H1 | Chothia | GFTISDY |
| 211 | SRP2900-H06 | CDR-H1 | Chothia | GFTIAAY |
| 212 | SRP2900-H07 | CDR-H1 | Chothia | GENIAYY |
| 213 | SRP2900-H08 | CDR-H1 | Chothia | GFTISDY |
| 214 | SRP2900-H09 | CDR-H1 | Chothia | GFTISDY |
| 215 | SRP2900-H10 | CDR-H1 | Chothia | GFTISDY |
| 216 | SRP2900-H11 | CDR-H1 | Chothia | GFTISSY |
| 218 | SRP2842-B01 | CDR-H1 | Chothia | GFSISSY |
| 224 | SRP2842-G04 | CDR-H1 | Chothia | GFSLSYY |
| 229 | SRP2901-B05_ | CDR-H1 | Chothia | GFSLSYY |
| | 2842-G04_HC2 | | | |
| 235 | SRP2901-D03_ | CDR-H1 | Chothia | GFSISSY |
| | 2842-B01_HC-3 | | | |
| 237 | SRP2901-E03_ | CDR-H1 | Chothia | GFSISSY |
| | 2842-B01_HC-3 | | | |
| 238 | SRP2901-F01_ | CDR-H1 | Chothia | GFSISSY |
| | 2842-B01_HC-1 | | | |
| 430 | SRP2799-A05 | CDR-H1 | Kabat | SYWIH |
| 439 | SRP2799-B03 | CDR-H1 | Kabat | DYWIH |
| 442 | SRP2799-B06 | CDR-H1 | Kabat | DYNIH |
| 454 | SRP2900-A01 | CDR-H1 | Kabat | SYWIH |
| 455 | SRP2900-A02 | CDR-H1 | Kabat | GYIIH |
| 456 | SRP2900-A03 | CDR-H1 | Kabat | SYWIH |
| 457 | SRP2900-A04 | CDR-H1 | Kabat | GYWIH |
| 458 | SRP2900-A05 | CDR-H1 | Kabat | DYWIH |
| 459 | SRP2900-A06 | CDR-H1 | Kabat | SYWIH |
| 460 | SRP2900-A07 | CDR-H1 | Kabat | SYWIH |
| 461 | SRP2900-A08 | CDR-H1 | Kabat | GYWIH |
| 462 | SRP2900-A09 | CDR-H1 | Kabat | DYWIH |
| 463 | SRP2900-A10 | CDR-H1 | Kabat | SYWIH |
| 464 | SRP2900-A11 | CDR-H1 | Kabat | DYVIH |
| 465 | SRP2900-B01 | CDR-H1 | Kabat | GYWIH |
| 466 | SRP2900-B02 | CDR-H1 | Kabat | DYWIH |
| 467 | SRP2900-B03 | CDR-H1 | Kabat | DYWIH |
| 468 | SRP2900-B04 | CDR-H1 | Kabat | EYWIH |
| 469 | SRP2900-B05 | CDR-H1 | Kabat | GYWIH |
| 470 | SRP2900-B06 | CDR-H1 | Kabat | SYWIH |
| 471 | SRP2900-B07 | CDR-H1 | Kabat | DYWIH |
| 472 | SRP2900-B08 | CDR-H1 | Kabat | SYWIH |
| 473 | SRP2900-B09 | CDR-H1 | Kabat | QYWIH |
| 474 | SRP2900-B10 | CDR-H1 | Kabat | AYWIH |
| 475 | SRP2900-B11 | CDR-H1 | Kabat | AYWIH |
| 476 | SRP2900-C01 | CDR-H1 | Kabat | SYWIH |
| 477 | SRP2900-C02 | CDR-H1 | Kabat | SYWIH |
| 478 | SRP2900-C03 | CDR-H1 | Kabat | GYWIH |
| 479 | SRP2900-C04 | CDR-H1 | Kabat | SYWIH |
| 480 | SRP2900-C05 | CDR-H1 | Kabat | GYWIH |
| 481 | SRP2900-C06 | CDR-H1 | Kabat | GYWIH |
| 482 | SRP2900-C07 | CDR-H1 | Kabat | GYWIH |
| 483 | SRP2900-C08 | CDR-H1 | Kabat | AYWIH |
| 484 | SRP2900-C09 | CDR-H1 | Kabat | DYIIH |
| 485 | SRP2900-C10 | CDR-H1 | Kabat | DYWIH |
| 486 | SRP2900-C11 | CDR-H1 | Kabat | QYWIH |
| 487 | SRP2900-D01 | CDR-H1 | Kabat | EYWIH |
| 488 | SRP2900-D02 | CDR-H1 | Kabat | DYWIH |
| 489 | SRP2900-D03 | CDR-H1 | Kabat | QYWIH |
| 490 | SRP2900-D04 | CDR-H1 | Kabat | DYWIH |
| 491 | SRP2900-D05 | CDR-H1 | Kabat | NYWIH |
| 492 | SRP2900-D06 | CDR-H1 | Kabat | DYWIH |
| 493 | SRP2900-D07 | CDR-H1 | Kabat | DYWIH |
| 494 | SRP2900-D08 | CDR-H1 | Kabat | DYWIH |
| 495 | SRP2900-D09 | CDR-H1 | Kabat | GYWIH |
| 496 | SRP2900-D10 | CDR-H1 | Kabat | DYWIH |
| 497 | SRP2900-D11 | CDR-H1 | Kabat | DYWIH |
| 498 | SRP2900-E01 | CDR-H1 | Kabat | DYWIH |
| 499 | SRP2900-E02 | CDR-H1 | Kabat | DYVIH |
| 500 | SRP2900-E03 | CDR-H1 | Kabat | DYWIH |
| 501 | SRP2900-E04 | CDR-H1 | Kabat | DYWIH |
| 502 | SRP2900-E05 | CDR-H1 | Kabat | GYWIH |
| 503 | SRP2900-E06 | CDR-H1 | Kabat | GYIIH |
| 504 | SRP2900-E07 | CDR-H1 | Kabat | QYWIH |
| 505 | SRP2900-E08 | CDR-H1 | Kabat | DYWIH |
| 506 | SRP2900-E09 | CDR-H1 | Kabat | QYWIH |
| 507 | SRP2900-E10 | CDR-H1 | Kabat | DYWIH |
| 508 | SRP2900-E11 | CDR-H1 | Kabat | YYIIH |
| 509 | SRP2900-F01 | CDR-H1 | Kabat | DYWIH |
| 510 | SRP2900-F02 | CDR-H1 | Kabat | VYIIH |
| 511 | SRP2900-F03 | CDR-H1 | Kabat | DYWIH |
| 512 | SRP2900-F04 | CDR-H1 | Kabat | DYWIH |
| 513 | SRP2900-F05 | CDR-H1 | Kabat | DYVIH |
| 514 | SRP2900-F06 | CDR-H1 | Kabat | IYTIH |
| 515 | SRP2900-F07 | CDR-H1 | Kabat | DYTIH |
| 516 | SRP2900-F08 | CDR-H1 | Kabat | YYIIH |
| 517 | SRP2900-F09 | CDR-H1 | Kabat | DYVIH |
| 518 | SRP2900-F10 | CDR-H1 | Kabat | DYVIH |
| 519 | SRP2900-F11 | CDR-H1 | Kabat | GYIIH |
| 520 | SRP2900-G01 | CDR-H1 | Kabat | DYTIH |
| 521 | SRP2900-G02 | CDR-H1 | Kabat | YYIIH |
| 522 | SRP2900-G03 | CDR-H1 | Kabat | NYIIH |
| 523 | SRP2900-G04 | CDR-H1 | Kabat | DYVIH |
| 524 | SRP2900-G05 | CDR-H1 | Kabat | YYVIH |
| 525 | SRP2900-G06 | CDR-H1 | Kabat | DYIIH |
| 526 | SRP2900-G07 | CDR-H1 | Kabat | DYVIH |
| 527 | SRP2900-G08 | CDR-H1 | Kabat | GYIIH |
| 528 | SRP2900-G09 | CDR-H1 | Kabat | DYIIH |
| 529 | SRP2900-G10 | CDR-H1 | Kabat | DYIIH |
| 530 | SRP2900-G11 | CDR-H1 | Kabat | DYIIH |
| 531 | SRP2900-H01 | CDR-H1 | Kabat | DYIIH |
| 532 | SRP2900-H02 | CDR-H1 | Kabat | DYIIH |
| 533 | SRP2900-H03 | CDR-H1 | Kabat | DYIIH |
| 534 | SRP2900-H04 | CDR-H1 | Kabat | HYIIH |
| 535 | SRP2900-H05 | CDR-H1 | Kabat | DYTIH |
| 536 | SRP2900-H06 | CDR-H1 | Kabat | AYIIH |
| 537 | SRP2900-H07 | CDR-H1 | Kabat | YYTIH |
| 538 | SRP2900-H08 | CDR-H1 | Kabat | DYVIH |
| 539 | SRP2900-H09 | CDR-H1 | Kabat | DYVIH |
| 540 | SRP2900-H10 | CDR-H1 | Kabat | DYVIH |
| 541 | SRP2900-H11 | CDR-H1 | Kabat | SYIIH |
| 543 | SRP2842-B01 | CDR-H1 | Kabat | SYDMS |
| 549 | SRP2842-G04 | CDR-H1 | Kabat | YYGVS |
| 554 | SRP2901-B05_ | CDR-H1 | Kabat | YYGVS |
| | 2842-G04_HC2 | | | |
| 560 | SRP2901-D03_ | CDR-H1 | Kabat | SYDMS |
| | 2842-B01_HC-3 | | | |
| 562 | SRP2901-E03_ | CDR-H1 | Kabat | SYDMS |
| | 2842-B01_HC-3 | | | |
| 563 | SRP2901-F01_ | CDR-H1 | Kabat | SYDMS |
| | 2842-B01_HC-1 | | | |
| 755 | SRP2799-A05 | CDR-H2 | Chothia | DPYSGS |
| 764 | SRP2799-B03 | CDR-H2 | Chothia | DPYNGY |
| 767 | SRP2799-B06 | CDR-H2 | Chothia | DPSNGY |
| 779 | SRP2900-A01 | CDR-H2 | Chothia | DPYNGA |
| 780 | SRP2900-A02 | CDR-H2 | Chothia | DPSNGY |
| 781 | SRP2900-A03 | CDR-H2 | Chothia | DPANGA |
| 782 | SRP2900-A04 | CDR-H2 | Chothia | DPYNGA |
| 783 | SRP2900-A05 | CDR-H2 | Chothia | DPYNGY |
| 784 | SRP2900-A06 | CDR-H2 | Chothia | DPFNGA |
| 785 | SRP2900-A07 | CDR-H2 | Chothia | DPYNGA |
| 786 | SRP2900-A08 | CDR-H2 | Chothia | DPYNGA |
| 787 | SRP2900-A09 | CDR-H2 | Chothia | DPYNGA |
| 788 | SRP2900-A10 | CDR-H2 | Chothia | DPYNGA |
| 789 | SRP2900-A11 | CDR-H2 | Chothia | DPSNGY |
| 790 | SRP2900-B01 | CDR-H2 | Chothia | DPYNGA |
| 791 | SRP2900-B02 | CDR-H2 | Chothia | DPYNGA |
| 792 | SRP2900-B03 | CDR-H2 | Chothia | DPYNGA |
| 793 | SRP2900-B04 | CDR-H2 | Chothia | DPYNGY |
| 794 | SRP2900-B05 | CDR-H2 | Chothia | DPYNGA |
| 795 | SRP2900-B06 | CDR-H2 | Chothia | DPYNGA |
| 796 | SRP2900-B07 | CDR-H2 | Chothia | DPYNGY |
| 797 | SRP2900-B08 | CDR-H2 | Chothia | DPYNGY |
| 798 | SRP2900-B09 | CDR-H2 | Chothia | DPYNGY |
| 799 | SRP2900-B10 | CDR-H2 | Chothia | DPYNGS |
| 800 | SRP2900-B11 | CDR-H2 | Chothia | DPSNGY |
| 801 | SRP2900-C01 | CDR-H2 | Chothia | DPSNGY |
| 802 | SRP2900-C02 | CDR-H2 | Chothia | DPYNGA |
| 803 | SRP2900-C03 | CDR-H2 | Chothia | DPYNGA |
| 804 | SRP2900-C04 | CDR-H2 | Chothia | DPYNGA |
| 805 | SRP2900-C05 | CDR-H2 | Chothia | DPYNGA |
| 806 | SRP2900-C06 | CDR-H2 | Chothia | DPYNGA |
| 807 | SRP2900-C07 | CDR-H2 | Chothia | DPYNGA |
| 808 | SRP2900-C08 | CDR-H2 | Chothia | DPYNGS |
| 809 | SRP2900-C09 | CDR-H2 | Chothia | DPSNGY |
| 810 | SRP2900-C10 | CDR-H2 | Chothia | DPYNGY |
| 811 | SRP2900-C11 | CDR-H2 | Chothia | DPYNGY |
| 812 | SRP2900-D01 | CDR-H2 | Chothia | DPANGE |
| 813 | SRP2900-D02 | CDR-H2 | Chothia | DPYNGY |
| 814 | SRP2900-D03 | CDR-H2 | Chothia | DPYNGY |
| 815 | SRP2900-D04 | CDR-H2 | Chothia | DPYNGY |
| 816 | SRP2900-D05 | CDR-H2 | Chothia | DPYNGY |
| 817 | SRP2900-D06 | CDR-H2 | Chothia | DPQNGW |
| 818 | SRP2900-D07 | CDR-H2 | Chothia | DPSNGY |
| 819 | SRP2900-D08 | CDR-H2 | Chothia | DPYNGY |
| 820 | SRP2900-D09 | CDR-H2 | Chothia | DPANGE |
| 821 | SRP2900-D10 | CDR-H2 | Chothia | DPKNGY |
| 822 | SRP2900-D11 | CDR-H2 | Chothia | DPYNGY |
| 823 | SRP2900-E01 | CDR-H2 | Chothia | DPSNGY |
| 824 | SRP2900-E02 | CDR-H2 | Chothia | DPSNGY |
| 825 | SRP2900-E03 | CDR-H2 | Chothia | DPQNGW |
| 826 | SRP2900-E04 | CDR-H2 | Chothia | DPYSGS |
| 827 | SRP2900-E05 | CDR-H2 | Chothia | DPANGE |
| 828 | SRP2900-E06 | CDR-H2 | Chothia | DPSNGY |
| 829 | SRP2900-E07 | CDR-H2 | Chothia | DPYNGY |
| 830 | SRP2900-E08 | CDR-H2 | Chothia | DPYNGY |
| 831 | SRP2900-E09 | CDR-H2 | Chothia | DPYNGY |
| 832 | SRP2900-E10 | CDR-H2 | Chothia | DPANGY |
| 833 | SRP2900-E11 | CDR-H2 | Chothia | DPSNGY |
| 834 | SRP2900-F01 | CDR-H2 | Chothia | DPYNGY |
| 835 | SRP2900-F02 | CDR-H2 | Chothia | DPSNGY |
| 836 | SRP2900-F03 | CDR-H2 | Chothia | DPYNGY |
| 837 | SRP2900-F04 | CDR-H2 | Chothia | DPYNGF |
| 838 | SRP2900-F05 | CDR-H2 | Chothia | DPSNGY |
| 839 | SRP2900-F06 | CDR-H2 | Chothia | DPSNGY |
| 840 | SRP2900-F07 | CDR-H2 | Chothia | DPSNGY |
| 841 | SRP2900-F08 | CDR-H2 | Chothia | DPSNGY |
| 842 | SRP2900-F09 | CDR-H2 | Chothia | DPSNGY |
| 843 | SRP2900-F10 | CDR-H2 | Chothia | DPSNGY |
| 844 | SRP2900-F11 | CDR-H2 | Chothia | DPSNGY |
| 845 | SRP2900-G01 | CDR-H2 | Chothia | DPSNGY |
| 846 | SRP2900-G02 | CDR-H2 | Chothia | DPSNGY |
| 847 | SRP2900-G03 | CDR-H2 | Chothia | DPSNGY |
| 848 | SRP2900-G04 | CDR-H2 | Chothia | DPSNGY |
| 849 | SRP2900-G05 | CDR-H2 | Chothia | DPSNGY |
| 850 | SRP2900-G06 | CDR-H2 | Chothia | DPSNGY |
| 851 | SRP2900-G07 | CDR-H2 | Chothia | DPSNGY |
| 852 | SRP2900-G08 | CDR-H2 | Chothia | DPSNGY |
| 853 | SRP2900-G09 | CDR-H2 | Chothia | DPSNGY |
| 854 | SRP2900-G10 | CDR-H2 | Chothia | DPSNGY |
| 855 | SRP2900-G11 | CDR-H2 | Chothia | DPSNGY |
| 856 | SRP2900-H01 | CDR-H2 | Chothia | DPSNGY |
| 857 | SRP2900-H02 | CDR-H2 | Chothia | DPSNGY |
| 858 | SRP2900-H03 | CDR-H2 | Chothia | DPSNGY |
| 859 | SRP2900-H04 | CDR-H2 | Chothia | DPSNGY |
| 860 | SRP2900-H05 | CDR-H2 | Chothia | DPSNGY |
| 861 | SRP2900-H06 | CDR-H2 | Chothia | DPSNGY |
| 862 | SRP2900-H07 | CDR-H2 | Chothia | DPSNGY |
| 863 | SRP2900-H08 | CDR-H2 | Chothia | DPSNGY |
| 864 | SRP2900-H09 | CDR-H2 | Chothia | DPSNGY |
| 865 | SRP2900-H10 | CDR-H2 | Chothia | DPSNGF |
| 866 | SRP2900-H11 | CDR-H2 | Chothia | DPSNGY |
| 868 | SRP2842-B01 | CDR-H2 | Chothia | IGSNGR |
| 874 | SRP2842-G04 | CDR-H2 | Chothia | DSSGR |
| 879 | SRP2901-B05_ | CDR-H2 | Chothia | DSSGR |
| | 2842-G04_HC2 | | | |
| 885 | SRP2901-D03_ | CDR-H2 | Chothia | IGSNGR |
| | 2842-B01_HC-3 | | | |
| 887 | SRP2901-E03_ | CDR-H2 | Chothia | IGSNGR |
| | 2842-B01_HC-3 | | | |
| 888 | SRP2901-F01_ | CDR-H2 | Chothia | IGSNGR |
| | 2842-B01_HC-1 | | | |
| 1080 | SRP2799-A05 | CDR-H2 | Kabat | YIDPYSGSTYYADSVKG |
| 1089 | SRP2799-B03 | CDR-H2 | Kabat | FIDPYNGYTNYADSVKG |
| 1092 | SRP2799-B06 | CDR-H2 | Kabat | YIDPSNGYTYYADSVKG |
| 1104 | SRP2900-A01 | CDR-H2 | Kabat | YIDPYNGATYYADSVKG |
| 1105 | SRP2900-A02 | CDR-H2 | Kabat | YIDPSNGYTYYADSVKG |
| 1106 | SRP2900-A03 | CDR-H2 | Kabat | YIDPANGATYYADSVKG |
| 1107 | SRP2900-A04 | CDR-H2 | Kabat | YIDPYNGATYYADSVKG |
| 1108 | SRP2900-A05 | CDR-H2 | Kabat | FIDPYNGYTNYADPVKS |
| 1109 | SRP2900-A06 | CDR-H2 | Kabat | YIDPFNGATYYADSVKG |
| 1110 | SRP2900-A07 | CDR-H2 | Kabat | YIDPYNGATYYADSVKG |
| 1111 | SRP2900-A08 | CDR-H2 | Kabat | YIDPYNGATYYADSVKG |
| 1112 | SRP2900-A09 | CDR-H2 | Kabat | YIDPYNGATYYADSVKG |
| 1113 | SRP2900-A10 | CDR-H2 | Kabat | YIDPYNGATYYADSVKG |
| 1114 | SRP2900-A11 | CDR-H2 | Kabat | YIDPSNGYTYYADSVKG |
| 1115 | SRP2900-B01 | CDR-H2 | Kabat | YIDPYNGATYYADSVKG |
| 1116 | SRP2900-B02 | CDR-H2 | Kabat | YIDPYNGATYYADSVKG |
| 1117 | SRP2900-B03 | CDR-H2 | Kabat | YIDPYNGATYYADSVKG |
| 1118 | SRP2900-B04 | CDR-H2 | Kabat | FIDPYNGYTGYADSVRG |
| 1119 | SRP2900-B05 | CDR-H2 | Kabat | YIDPYNGATYYADSVKG |
| 1120 | SRP2900-B06 | CDR-H2 | Kabat | YIDPYNGATYYADSVKG |
| 1121 | SRP2900-B07 | CDR-H2 | Kabat | FIDPYNGYTGYADSVKG |
| 1122 | SRP2900-B08 | CDR-H2 | Kabat | YIDPYNGYTYYADSVKG |
| 1123 | SRP2900-B09 | CDR-H2 | Kabat | FIDPYNGYTNYADSVKG |
| 1124 | SRP2900-B10 | CDR-H2 | Kabat | YIDPYNGSTYYADSVKG |
| 1125 | SRP2900-B11 | CDR-H2 | Kabat | YIDPSNGYTYYADSVKG |
| 1126 | SRP2900-C01 | CDR-H2 | Kabat | YIDPSNGYTYYADSVKG |
| 1127 | SRP2900-C02 | CDR-H2 | Kabat | YIDPYNGATYYADSVKG |
| 1128 | SRP2900-C03 | CDR-H2 | Kabat | YIDPYNGATYYADSVKG |
| 1129 | SRP2900-C04 | CDR-H2 | Kabat | YIDPYNGATYYADSVKG |
| 1130 | SRP2900-C05 | CDR-H2 | Kabat | YIDPYNGATYYADSVKG |
| 1131 | SRP2900-C06 | CDR-H2 | Kabat | YIDPYNGATFYADSVKG |
| 1132 | SRP2900-C07 | CDR-H2 | Kabat | YIDPYNGATYYADSVKG |
| 1133 | SRP2900-C08 | CDR-H2 | Kabat | YIDPYNGSTYHADSVKG |
| 1134 | SRP2900-C09 | CDR-H2 | Kabat | YIDPSNGYTYHADSVKG |
| 1135 | SRP2900-C10 | CDR-H2 | Kabat | YIDPYNGYTYYADSVNG |
| 1136 | SRP2900-C11 | CDR-H2 | Kabat | YIDPYNGYTYYADSVKG |
| 1137 | SRP2900-D01 | CDR-H2 | Kabat | YIDPANGFTYYADSVKG |
| 1138 | SRP2900-D02 | CDR-H2 | Kabat | YIDPYNGYTYYADSVKG |
| 1139 | SRP2900-D03 | CDR-H2 | Kabat | YIDPYNGYTYYADSVKG |
| 1140 | SRP2900-D04 | CDR-H2 | Kabat | FIDPYNGYTYHADSVEG |
| 1141 | SRP2900-D05 | CDR-H2 | Kabat | YIDPYNGYTYYADSVKG |
| 1142 | SRP2900-D06 | CDR-H2 | Kabat | YIDPQNGWTYYADSVKG |
| 1143 | SRP2900-D07 | CDR-H2 | Kabat | YIDPSNGYTYYADSVKG |
| 1144 | SRP2900-D08 | CDR-H2 | Kabat | YIDPYNGYTYYADSVKG |
| 1145 | SRP2900-D09 | CDR-H2 | Kabat | YIDPANGFTYYADSVKG |
| 1146 | SRP2900-D10 | CDR-H2 | Kabat | FIDPKNGYTYYADSVKG |
| 1147 | SRP2900-D11 | CDR-H2 | Kabat | YIDPYNGYTYYADSVKG |
| 1148 | SRP2900-E01 | CDR-H2 | Kabat | YIDPSNGYTYYADSVKG |
| 1149 | SRP2900-E02 | CDR-H2 | Kabat | YIDPSNGYTYYADPVKG |
| 1150 | SRP2900-E03 | CDR-H2 | Kabat | YIDPQNGWTYYADSVKG |
| 1151 | SRP2900-E04 | CDR-H2 | Kabat | YIDPYSGSTFYADSVKG |
| 1152 | SRP2900-E05 | CDR-H2 | Kabat | YIDPANGFTYYADSVKG |
| 1153 | SRP2900-E06 | CDR-H2 | Kabat | YIDPSNGYTYHADSVKG |
| 1154 | SRP2900-E07 | CDR-H2 | Kabat | YIDPYNGYTYYADSVKG |
| 1155 | SRP2900-E08 | CDR-H2 | Kabat | YIDPYNGYTYYADSVKG |
| 1156 | SRP2900-E09 | CDR-H2 | Kabat | YIDPYNGYTYYADSVKG |
| 1157 | SRP2900-E10 | CDR-H2 | Kabat | YIDPANGYTYYADSVKG |
| 1158 | SRP2900-E11 | CDR-H2 | Kabat | YIDPSNGYTYYADSVKG |
| 1159 | SRP2900-F01 | CDR-H2 | Kabat | YIDPYNGYTYYADSVRG |
| 1160 | SRP2900-F02 | CDR-H2 | Kabat | YIDPSNGYTYYADSVKG |
| 1161 | SRP2900-F03 | CDR-H2 | Kabat | YIDPYNGYTYYADSVKG |
| 1162 | SRP2900-F04 | CDR-H2 | Kabat | YIDPYNGFTYYADSVKG |
| 1163 | SRP2900-F05 | CDR-H2 | Kabat | YIDPSNGYTYYADSVKG |
| 1164 | SRP2900-F06 | CDR-H2 | Kabat | YIDPSNGYTYYADSVKG |
| 1165 | SRP2900-F07 | CDR-H2 | Kabat | YIDPSNGYTYHADSVKG |
| 1166 | SRP2900-F08 | CDR-H2 | Kabat | YIDPSNGYTYYADSVKG |
| 1167 | SRP2900-F09 | CDR-H2 | Kabat | YIDPSNGYTYYADSVKG |
| 1168 | SRP2900-F10 | CDR-H2 | Kabat | YIDPSNGYTYYADSVKG |
| 1169 | SRP2900-F11 | CDR-H2 | Kabat | YIDPSNGYTFYADSVKG |
| 1170 | SRP2900-G01 | CDR-H2 | Kabat | YIDPSNGYTFYADSVKG |
| 1171 | SRP2900-G02 | CDR-H2 | Kabat | YIDPSNGYTYYADSVKG |
| 1172 | SRP2900-G03 | CDR-H2 | Kabat | YIDPSNGYTYYADSVKG |
| 1173 | SRP2900-G04 | CDR-H2 | Kabat | YIDPSNGYTYYADSVKG |
| 1174 | SRP2900-G05 | CDR-H2 | Kabat | YIDPSNGYTYYADSVKG |
| 1175 | SRP2900-G06 | CDR-H2 | Kabat | YIDPSNGYTYYADSVKG |
| 1176 | SRP2900-G07 | CDR-H2 | Kabat | YIDPSNGYTYYADSVKG |
| 1177 | SRP2900-G08 | CDR-H2 | Kabat | YIDPSNGYTYYADSVKG |
| 1178 | SRP2900-G09 | CDR-H2 | Kabat | YIDPSNGYTYYADSVKG |
| 1179 | SRP2900-G10 | CDR-H2 | Kabat | YIDPSNGYTYYADSVKG |
| 1180 | SRP2900-G11 | CDR-H2 | Kabat | YIDPSNGYTDYADSVKG |
| 1181 | SRP2900-H01 | CDR-H2 | Kabat | YIDPSNGYTYYADSVKG |
| 1182 | SRP2900-H02 | CDR-H2 | Kabat | YIDPSNGYTYYADSVKG |
| 1183 | SRP2900-H03 | CDR-H2 | Kabat | YIDPSNGYTYHADSVKG |
| 1184 | SRP2900-H04 | CDR-H2 | Kabat | YIDPSNGYTYYADSVKG |
| 1185 | SRP2900-H05 | CDR-H2 | Kabat | YIDPSNGYTYYADSVKG |
| 1186 | SRP2900-H06 | CDR-H2 | Kabat | YIDPSNGYTYYADSVKG |
| 1187 | SRP2900-H07 | CDR-H2 | Kabat | YIDPSNGYTYYADSVKG |
| 1188 | SRP2900-H08 | CDR-H2 | Kabat | YIDPSNGYTFYADSVKG |
| 1189 | SRP2900-H09 | CDR-H2 | Kabat | YIDPSNGYTYYADSVKG |
| 1190 | SRP2900-H10 | CDR-H2 | Kabat | YIDPSNGFTYYADSVKG |
| 1191 | SRP2900-H11 | CDR-H2 | Kabat | YIDPSNGYTYYADSVKG |
| 1193 | SRP2842-B01 | CDR-H2 | Kabat | IIGSNGRTYYPNWAKS |
| 1199 | SRP2842-G04 | CDR-H2 | Kabat | TIDSSGRTYYASWANG |
| 1204 | SRP2901-B05_ | CDR-H2 | Kabat | TIDSSGRTYYNPSLKS |
| | 2842-G04_HC2 | | | |
| 1210 | SRP2901-D03_ | CDR-H2 | Kabat | IIGSNGRTYYPDSVKG |
| | 2842-B01_HC-3 | | | |
| 1212 | SRP2901-E03_ | CDR-H2 | Kabat | IIGSNGRTYYPDSVKG |
| | 2842-B01_HC-3 | | | |
| 1213 | SRP2901-F01_ | CDR-H2 | Kabat | IIGSNGRTYYADSVKG |
| | 2842-B01_HC-1 | | | |
| 1405 | SRP2799-A05 | CDR-H3 | Chothia | SDYYYVDEY |
| 1414 | SRP2799-B03 | CDR-H3 | Chothia | GYGSWPDY |
| 1417 | SRP2799-B06 | CDR-H3 | Chothia | DDQSVSSP |
| 1429 | SRP2900-A01 | CDR-H3 | Chothia | ADYYYVDEYFDY |
| 1430 | SRP2900-A02 | CDR-H3 | Chothia | DDQSLSSPFDY |
| 1431 | SRP2900-A03 | CDR-H3 | Chothia | ADYFYVDEYLDY |
| 1432 | SRP2900-A04 | CDR-H3 | Chothia | ADYFYIDEYWDY |
| 1433 | SRP2900-A05 | CDR-H3 | Chothia | ADYYYVDEYWDY |
| 1434 | SRP2900-A06 | CDR-H3 | Chothia | ADYYYVDEYWDY |
| 1435 | SRP2900-A07 | CDR-H3 | Chothia | SDYYYVDEFFDY |
| 1436 | SRP2900-A08 | CDR-H3 | Chothia | ADYYYVDEYWDY |
| 1437 | SRP2900-A09 | CDR-H3 | Chothia | ADYYYVDEYWDY |
| 1438 | SRP2900-A10 | CDR-H3 | Chothia | ADYYYVDEYLDY |
| 1439 | SRP2900-A11 | CDR-H3 | Chothia | DDQSFTSPFDY |
| 1440 | SRP2900-B01 | CDR-H3 | Chothia | ADYYYVDEYLDY |
| 1441 | SRP2900-B02 | CDR-H3 | Chothia | ADYYYVDEYWDY |
| 1442 | SRP2900-B03 | CDR-H3 | Chothia | ADYYYVDEYWDY |
| 1443 | SRP2900-B04 | CDR-H3 | Chothia | ADYYYVDEYWDY |
| 1444 | SRP2900-B05 | CDR-H3 | Chothia | ADYYGVDEFLDY |
| 1445 | SRP2900-B06 | CDR-H3 | Chothia | ADYHGVDEYWDY |
| 1446 | SRP2900-B07 | CDR-H3 | Chothia | ADYHGVDEYWDY |
| 1447 | SRP2900-B08 | CDR-H3 | Chothia | ADYYYVDEYFDY |
| 1448 | SRP2900-B09 | CDR-H3 | Chothia | ADYYYVDEYWDY |
| 1449 | SRP2900-B10 | CDR-H3 | Chothia | ADYYYVDEYWDY |
| 1450 | SRP2900-B11 | CDR-H3 | Chothia | ADYHGVDEYWDY |
| 1451 | SRP2900-C01 | CDR-H3 | Chothia | ADYYYVDEYWDY |
| 1452 | SRP2900-C02 | CDR-H3 | Chothia | ADYYYVDEYWDY |
| 1453 | SRP2900-C03 | CDR-H3 | Chothia | ADYYYVDEYWDY |
| 1454 | SRP2900-C04 | CDR-H3 | Chothia | ADYYYVDEYWDY |
| 1455 | SRP2900-C05 | CDR-H3 | Chothia | ADYHGVDEYWDY |
| 1456 | SRP2900-C06 | CDR-H3 | Chothia | ADYYYVDEYWDY |
| 1457 | SRP2900-C07 | CDR-H3 | Chothia | ADYYYVDEYFDY |
| 1458 | SRP2900-C08 | CDR-H3 | Chothia | ADYYYVDEYWDY |
| 1459 | SRP2900-C09 | CDR-H3 | Chothia | DDQSFSSPFDY |
| 1460 | SRP2900-C10 | CDR-H3 | Chothia | DYGSYLVSLDY |
| 1461 | SRP2900-C11 | CDR-H3 | Chothia | DYGSWRIRLDY |
| 1462 | SRP2900-D01 | CDR-H3 | Chothia | DYGSLQVFLDY |
| 1463 | SRP2900-D02 | CDR-H3 | Chothia | DYGSLQVFIDH |
| 1464 | SRP2900-D03 | CDR-H3 | Chothia | DYGSLQVQLDY |
| 1465 | SRP2900-D04 | CDR-H3 | Chothia | DYGSLQVQLDY |
| 1466 | SRP2900-D05 | CDR-H3 | Chothia | DYGSLEVSLDY |
| 1467 | SRP2900-D06 | CDR-H3 | Chothia | DYGSLTINLDY |
| 1468 | SRP2900-D07 | CDR-H3 | Chothia | DYGSLEVSLDY |
| 1469 | SRP2900-D08 | CDR-H3 | Chothia | DYGSLEVSLDY |
| 1470 | SRP2900-D09 | CDR-H3 | Chothia | DYGSIQVFLDY |
| 1471 | SRP2900-D10 | CDR-H3 | Chothia | DYGSLQVFIDY |
| 1472 | SRP2900-D11 | CDR-H3 | Chothia | DYGSLQVQLDY |
| 1473 | SRP2900-E01 | CDR-H3 | Chothia | DYGSLQVFIDY |
| 1474 | SRP2900-E02 | CDR-H3 | Chothia | DPQSFYIAFDY |
| 1475 | SRP2900-E03 | CDR-H3 | Chothia | DYGSLEVSLDY |
| 1476 | SRP2900-E04 | CDR-H3 | Chothia | ADYYYVDEYLDY |
| 1477 | SRP2900-E05 | CDR-H3 | Chothia | DYGSVRVFLDY |
| 1478 | SRP2900-E06 | CDR-H3 | Chothia | DDQSVSSPFDY |
| 1479 | SRP2900-E07 | CDR-H3 | Chothia | DYGSLRVSLDY |
| 1480 | SRP2900-E08 | CDR-H3 | Chothia | DYGSLQVFIDY |
| 1481 | SRP2900-E09 | CDR-H3 | Chothia | DYGSFRVQLDY |
| 1482 | SRP2900-E10 | CDR-H3 | Chothia | DYGSLHVQLDY |
| 1483 | SRP2900-E11 | CDR-H3 | Chothia | DVQSVSGEFDY |
| 1484 | SRP2900-F01 | CDR-H3 | Chothia | DYGSLQVFIDY |
| 1485 | SRP2900-F02 | CDR-H3 | Chothia | DDQSVSSPFDY |
| 1486 | SRP2900-F03 | CDR-H3 | Chothia | DYGSLQVFIDY |
| 1487 | SRP2900-F04 | CDR-H3 | Chothia | DYGSLQVFIDY |
| 1488 | SRP2900-F05 | CDR-H3 | Chothia | DDQSLSSPFDY |
| 1489 | SRP2900-F06 | CDR-H3 | Chothia | DPQSISSPFDY |
| 1490 | SRP2900-F07 | CDR-H3 | Chothia | DPQSVSSPFDY |
| 1491 | SRP2900-F08 | CDR-H3 | Chothia | DDQSLSSPFDY |
| 1492 | SRP2900-F09 | CDR-H3 | Chothia | DDQSISSPLDY |
| 1493 | SRP2900-F10 | CDR-H3 | Chothia | DPQSLFYPFDY |
| 1494 | SRP2900-F11 | CDR-H3 | Chothia | DDQSLSSPFDY |
| 1495 | SRP2900-G01 | CDR-H3 | Chothia | DIQSVSGEFDY |
| 1496 | SRP2900-G02 | CDR-H3 | Chothia | DDQSVSSPFEY |
| 1497 | SRP2900-G03 | CDR-H3 | Chothia | DPQSLSSPFDY |
| 1498 | SRP2900-G04 | CDR-H3 | Chothia | DPQSLSSPFDY |
| 1499 | SRP2900-G05 | CDR-H3 | Chothia | DDQSLSSPFDY |
| 1500 | SRP2900-G06 | CDR-H3 | Chothia | DDQSLSSPFDY |
| 1501 | SRP2900-G07 | CDR-H3 | Chothia | DPQSLTSPFDY |
| 1502 | SRP2900-G08 | CDR-H3 | Chothia | DIQSVSGEFDY |
| 1503 | SRP2900-G09 | CDR-H3 | Chothia | DPQSLTSPFDY |
| 1504 | SRP2900-G10 | CDR-H3 | Chothia | DIQSVSGEFDY |
| 1505 | SRP2900-G11 | CDR-H3 | Chothia | DIQSVSGEFDY |
| 1506 | SRP2900-H01 | CDR-H3 | Chothia | DPQSVGSEFDY |
| 1507 | SRP2900-H02 | CDR-H3 | Chothia | DIQSVSGEFDY |
| 1508 | SRP2900-H03 | CDR-H3 | Chothia | DPQSLSSPFDY |
| 1509 | SRP2900-H04 | CDR-H3 | Chothia | DIQSVSGEFDY |
| 1510 | SRP2900-H05 | CDR-H3 | Chothia | DIQSVSGEFDY |
| 1511 | SRP2900-H06 | CDR-H3 | Chothia | DDQSFSSPFDY |
| 1512 | SRP2900-H07 | CDR-H3 | Chothia | DPQSFSSPFDY |
| 1513 | SRP2900-H08 | CDR-H3 | Chothia | DPQSLSSPFDY |
| 1514 | SRP2900-H09 | CDR-H3 | Chothia | DDQSISSPLDY |
| 1515 | SRP2900-H10 | CDR-H3 | Chothia | DIQSVSGEFDY |
| 1516 | SRP2900-H11 | CDR-H3 | Chothia | DPQTLTSPFDY |
| 1518 | SRP2842-B01 | CDR-H3 | Chothia | GLYDGTGNI |
| 1524 | SRP2842-G04 | CDR-H3 | Chothia | DFYGWNSGALDI |
| 1529 | SRP2901-B05_ | CDR-H3 | Chothia | DFYGWNSGALDI |
| | 2842-G04_HC2 | | | |
| 1535 | SRP2901-D03_ | CDR-H3 | Chothia | GLYDGTGNI |
| | 2842-B01_HC-3 | | | |
| 1537 | SRP2901-E03_ | CDR-H3 | Chothia | GLYDGTGNI |
| | 2842-B01_HC-3 | | | |
| 1538 | SRP2901-F01_ | CDR-H3 | Chothia | GLYDGTGNI |
| | 2842-B01_HC-1 | | | |
| 1730 | SRP2799-A05 | CDR-H3 | Kabat | SDYYYVDEYMDY |
| 1739 | SRP2799-B03 | CDR-H3 | Kabat | GYGSWPDYLDY |
| 1742 | SRP2799-B06 | CDR-H3 | Kabat | DDQSVSSPFDY |
| 1754 | SRP2900-A01 | CDR-H3 | Kabat | ADYYYVDEYFDY |
| 1755 | SRP2900-A02 | CDR-H3 | Kabat | DDQSLSSPFDY |
| 1756 | SRP2900-A03 | CDR-H3 | Kabat | ADYFYVDEYLDY |
| 1757 | SRP2900-A04 | CDR-H3 | Kabat | ADYFYIDEYWDY |
| 1758 | SRP2900-A05 | CDR-H3 | Kabat | ADYYYVDEYWDY |
| 1759 | SRP2900-A06 | CDR-H3 | Kabat | ADYYYVDEYWDY |
| 1760 | SRP2900-A07 | CDR-H3 | Kabat | SDYYYVDEFFDY |
| 1761 | SRP2900-A08 | CDR-H3 | Kabat | ADYYYVDEYWDY |
| 1762 | SRP2900-A09 | CDR-H3 | Kabat | ADYYYVDEYWDY |
| 1763 | SRP2900-A10 | CDR-H3 | Kabat | ADYYYVDEYLDY |
| 1764 | SRP2900-A11 | CDR-H3 | Kabat | DDQSFTSPFDY |
| 1765 | SRP2900-B01 | CDR-H3 | Kabat | ADYYYVDEYLDY |
| 1766 | SRP2900-B02 | CDR-H3 | Kabat | ADYYYVDEYWDY |
| 1767 | SRP2900-B03 | CDR-H3 | Kabat | ADYYYVDEYWDY |
| 1768 | SRP2900-B04 | CDR-H3 | Kabat | ADYYYVDEYWDY |
| 1769 | SRP2900-B05 | CDR-H3 | Kabat | ADYYGVDEFLDY |
| 1770 | SRP2900-B06 | CDR-H3 | Kabat | ADYHGVDEYWDY |
| 1771 | SRP2900-B07 | CDR-H3 | Kabat | ADYHGVDEYWDY |
| 1772 | SRP2900-B08 | CDR-H3 | Kabat | ADYYYVDEYFDY |
| 1773 | SRP2900-B09 | CDR-H3 | Kabat | ADYYYVDEYWDY |
| 1774 | SRP2900-B10 | CDR-H3 | Kabat | ADYYYVDEYWDY |
| 1775 | SRP2900-B11 | CDR-H3 | Kabat | ADYHGVDEYWDY |
| 1776 | SRP2900-C01 | CDR-H3 | Kabat | ADYYYVDEYWDY |
| 1777 | SRP2900-C02 | CDR-H3 | Kabat | ADYYYVDEYWDY |
| 1778 | SRP2900-C03 | CDR-H3 | Kabat | ADYYYVDEYWDY |
| 1779 | SRP2900-C04 | CDR-H3 | Kabat | ADYYYVDEYWDY |
| 1780 | SRP2900-C05 | CDR-H3 | Kabat | ADYHGVDEYWDY |
| 1781 | SRP2900-C06 | CDR-H3 | Kabat | ADYYYVDEYWDY |
| 1782 | SRP2900-C07 | CDR-H3 | Kabat | ADYYYVDEYFDY |
| 1783 | SRP2900-C08 | CDR-H3 | Kabat | ADYYYVDEYWDY |
| 1784 | SRP2900-C09 | CDR-H3 | Kabat | DDQSFSSPFDY |
| 1785 | SRP2900-C10 | CDR-H3 | Kabat | DYGSYLVSLDY |
| 1786 | SRP2900-C11 | CDR-H3 | Kabat | DYGSWRIRLDY |
| 1787 | SRP2900-D01 | CDR-H3 | Kabat | DYGSLQVFLDY |
| 1788 | SRP2900-D02 | CDR-H3 | Kabat | DYGSLQVFIDH |
| 1789 | SRP2900-D03 | CDR-H3 | Kabat | DYGSLQVQLDY |
| 1790 | SRP2900-D04 | CDR-H3 | Kabat | DYGSLQVQLDY |
| 1791 | SRP2900-D05 | CDR-H3 | Kabat | DYGSLEVSLDY |
| 1792 | SRP2900-D06 | CDR-H3 | Kabat | DYGSLTINLDY |
| 1793 | SRP2900-D07 | CDR-H3 | Kabat | DYGSLEVSLDY |
| 1794 | SRP2900-D08 | CDR-H3 | Kabat | DYGSLEVSLDY |
| 1795 | SRP2900-D09 | CDR-H3 | Kabat | DYGSIQVFLDY |
| 1796 | SRP2900-D10 | CDR-H3 | Kabat | DYGSLQVFIDY |
| 1797 | SRP2900-D11 | CDR-H3 | Kabat | DYGSLQVQLDY |
| 1798 | SRP2900-E01 | CDR-H3 | Kabat | DYGSLQVFIDY |
| 1799 | SRP2900-E02 | CDR-H3 | Kabat | DPQSFYIAFDY |
| 1800 | SRP2900-E03 | CDR-H3 | Kabat | DYGSLEVSLDY |
| 1801 | SRP2900-E04 | CDR-H3 | Kabat | ADYYYVDEYLDY |
| 1802 | SRP2900-E05 | CDR-H3 | Kabat | DYGSVRVFLDY |
| 1803 | SRP2900-E06 | CDR-H3 | Kabat | DDQSVSSPFDY |
| 1804 | SRP2900-E07 | CDR-H3 | Kabat | DYGSLRVSLDY |
| 1805 | SRP2900-E08 | CDR-H3 | Kabat | DYGSLQVFIDY |
| 1806 | SRP2900-E09 | CDR-H3 | Kabat | DYGSFRVQLDY |
| 1807 | SRP2900-E10 | CDR-H3 | Kabat | DYGSLHVQLDY |
| 1808 | SRP2900-E11 | CDR-H3 | Kabat | DVQSVSGEFDY |
| 1809 | SRP2900-F01 | CDR-H3 | Kabat | DYGSLQVFIDY |
| 1810 | SRP2900-F02 | CDR-H3 | Kabat | DDQSVSSPFDY |
| 1811 | SRP2900-F03 | CDR-H3 | Kabat | DYGSLQVFIDY |
| 1812 | SRP2900-F04 | CDR-H3 | Kabat | DYGSLQVFIDY |
| 1813 | SRP2900-F05 | CDR-H3 | Kabat | DDQSLSSPFDY |
| 1814 | SRP2900-F06 | CDR-H3 | Kabat | DPQSISSPFDY |
| 1815 | SRP2900-F07 | CDR-H3 | Kabat | DPQSVSSPFDY |
| 1816 | SRP2900-F08 | CDR-H3 | Kabat | DDQSLSSPFDY |
| 1817 | SRP2900-F09 | CDR-H3 | Kabat | DDQSISSPLDY |
| 1818 | SRP2900-F10 | CDR-H3 | Kabat | DPQSLFYPFDY |
| 1819 | SRP2900-F11 | CDR-H3 | Kabat | DDQSLSSPFDY |
| 1820 | SRP2900-G01 | CDR-H3 | Kabat | DIQSVSGEFDY |
| 1821 | SRP2900-G02 | CDR-H3 | Kabat | DDQSVSSPFEY |
| 1822 | SRP2900-G03 | CDR-H3 | Kabat | DPQSLSSPFDY |
| 1823 | SRP2900-G04 | CDR-H3 | Kabat | DPQSLSSPFDY |
| 1824 | SRP2900-G05 | CDR-H3 | Kabat | DDQSLSSPFDY |
| 1825 | SRP2900-G06 | CDR-H3 | Kabat | DDQSLSSPFDY |
| 1826 | SRP2900-G07 | CDR-H3 | Kabat | DPQSLTSPFDY |
| 1827 | SRP2900-G08 | CDR-H3 | Kabat | DIQSVSGEFDY |
| 1828 | SRP2900-G09 | CDR-H3 | Kabat | DPQSLTSPFDY |
| 1829 | SRP2900-G10 | CDR-H3 | Kabat | DIQSVSGEFDY |
| 1830 | SRP2900-G11 | CDR-H3 | Kabat | DIQSVSGEFDY |
| 1831 | SRP2900-H01 | CDR-H3 | Kabat | DPQSVGSEFDY |
| 1832 | SRP2900-H02 | CDR-H3 | Kabat | DIQSVSGEFDY |
| 1833 | SRP2900-H03 | CDR-H3 | Kabat | DPQSLSSPFDY |
| 1834 | SRP2900-H04 | CDR-H3 | Kabat | DIQSVSGEFDY |
| 1835 | SRP2900-H05 | CDR-H3 | Kabat | DIQSVSGEFDY |
| 1836 | SRP2900-H06 | CDR-H3 | Kabat | DDQSFSSPFDY |
| 1837 | SRP2900-H07 | CDR-H3 | Kabat | DPQSFSSPFDY |
| 1838 | SRP2900-H08 | CDR-H3 | Kabat | DPQSLSSPFDY |
| 1839 | SRP2900-H09 | CDR-H3 | Kabat | DDQSISSPLDY |
| 1840 | SRP2900-H10 | CDR-H3 | Kabat | DIQSVSGEFDY |
| 1841 | SRP2900-H11 | CDR-H3 | Kabat | DPQTLTSPFDY |
| 1843 | SRP2842-B01 | CDR-H3 | Kabat | GLYDGTGNI |
| 1849 | SRP2842-G04 | CDR-H3 | Kabat | DFYG |
| 1854 | SRP2901-B05_ | CDR-H3 | Kabat | DFYGWNSGALDI |
| | 2842-G04_HC2 | | | |
| 1860 | SRP2901-D03_ | CDR-H3 | Kabat | GLYDGTGNI |
| | 2842-B01_HC-3 | | | |
| 1862 | SRP2901-E03_ | CDR-H3 | Kabat | GLYDGTGNI |
| | 2842-B01_HC-3 | | | |
| 1863 | SRP2901-F01_ | CDR-H3 | Kabat | GLYDGTGNI |
| | 2842-B01_HC-1 | | | |
| 1954 | SRP2842-B01 | CDR-L1 | Chothia | QASQSVYGNNWLS |
| 1960 | SRP2842-G04 | CDR-L1 | Chothia | QASQSVYSNKYLS |
| 1965 | SRP2901-B05_ | CDR-L1 | Chothia | RASQSVYSNKYLS |
| | 2842-G04_LC2 | | | |
| 1971 | SRP2901-D03_ | CDR-L1 | Chothia | RASQSVYGNNWLS |
| | 2842-B01_LC3 | | | |
| 1973 | SRP2901-E03_ | CDR-L1 | Chothia | RASQSVYGNNWLS |
| | 2842-B01_LC3 | | | |
| 1974 | SRP2901-F01_ | CDR-L1 | Chothia | QASQSVYGNNWLS |
| | 2842-B01_LC1 | | | |
| 2064 | trastuzumab | CDR-L1 | Chothia | RASQDVNTAVA |
| 2066 | SRP2842-B01 | CDR-L1 | Kabat | QASQSVYGNNWLS |
| 2072 | SRP2842-G04 | CDR-L1 | Kabat | QASQSVYSNKYLS |
| 2077 | SRP2901-B05_ | CDR-L1 | Kabat | RASQSVYSNKYLS |
| | 2842-G04_LC2 | | | |
| 2083 | SRP2901-D03_ | CDR-L1 | Kabat | RASQSVYGNNWLS |
| | 2842-B01_LC3 | | | |
| 2085 | SRP2901-E03_ | CDR-L1 | Kabat | RASQSVYGNNWLS |
| | 2842-B01_LC3 | | | |
| 2086 | SRP2901-F01_ | CDR-L1 | Kabat | QASQSVYGNNWLS |
| | 2842-B01_LC1 | | | |
| 2176 | trastuzumab | CDR-L1 | Kabat | RASQDVNTAVA |
| 2178 | SRP2842-B01 | CDR-L2 | Chothia | GASKLAS |
| 2184 | SRP2842-G04 | CDR-L2 | Chothia | KASTLAS |
| 2189 | SRP2901-B05_ | CDR-L2 | Chothia | KASTLAS |
| | 2842-G04_LC2 | | | |
| 2195 | SRP2901-D03_ | CDR-L2 | Chothia | GASKLAS |
| | 2842-B01_LC3 | | | |
| 2197 | SRP2901-E03_ | CDR-L2 | Chothia | GASKLAT |
| | 2842-B01_LC3 | | | |
| 2198 | SRP2901-F01_ | CDR-L2 | Chothia | GASKLAS |
| | 2842-B01_LC1 | | | |
| 2288 | trastuzumab | CDR-L2 | Chothia | SASFLYS |
| 2290 | SRP2842-B01 | CDR-L2 | Kabat | GASKLAS |
| 2296 | SRP2842-G04 | CDR-L2 | Kabat | KASTLAS |
| 2301 | SRP2901-B05_ | CDR-L2 | Kabat | KASTLAS |
| | 2842-G04_LC2 | | | |
| 2307 | SRP2901-D03_ | CDR-L2 | Kabat | GASKLAS |
| | 2842-B01_LC3 | | | |
| 2309 | SRP2901-E03_ | CDR-L2 | Kabat | GASKLAT |
| | 2842-B01_LC3 | | | |
| 2310 | SRP2901-F01_ | CDR-L2 | Kabat | GASKLAS |
| | 2842-B01_LC1 | | | |
| 2400 | trastuzumab | CDR-L2 | Kabat | SASFLYS |
| 2402 | SRP2842-B01 | CDR-L3 | Chothia | QGTYYSGDWYFA |
| 2408 | SRP2842-G04 | CDR-L3 | Chothia | AAAYSDDSDTA |
| 2413 | SRP2901-B05_ | CDR-L3 | Chothia | AAAYSDDSDTA |
| | 2842-G04_LC2 | | | |
| 2419 | SRP2901-D03_ | CDR-L3 | Chothia | QGTYYSGDWYFA |
| | 2842-B01_LC3 | | | |
| 2421 | SRP2901-E03_ | CDR-L3 | Chothia | QGTYYSGDWYFA |
| | 2842-B01_LC3 | | | |
| 2422 | SRP2901-F01_ | CDR-L3 | Chothia | QGTYYSGDWYFA |
| | 2842-B01_LC1 | | | |
| 2512 | trastuzumab | CDR-L3 | Chothia | QQHYTTPPT |
| 2514 | SRP2842-B01 | CDR-L3 | Kabat | QGTYYSGDWYFA |
| 2520 | SRP2842-G04 | CDR-L3 | Kabat | AAAYSDDSDTA |
| 2525 | SRP2901-B05_ | CDR-L3 | Kabat | AAAYSDDSDTA |
| | 2842-G04_LC2 | | | |
| 2531 | SRP2901-D03_ | CDR-L3 | Kabat | QGTYYSGDWYFA |
| | 2842-B01_LC3 | | | |
| 2533 | SRP2901-E03_ | CDR-L3 | Kabat | QGTYYSGDWYFA |
| | 2842-B01_LC3 | | | |
| 2534 | SRP2901-F01_ | CDR-L3 | Kabat | QGTYYSGDWYFA |
| | 2842-B01_LC1 | | | |
| 2624 | Trastuzumab | CDR-L3 | Kabat | QQHYTTPPT |
| 2727 | SRP2799-A05 | V_{H} | | MAEVQLVESGGGLVQPGGSLRLSCAA |
| | | | | SGFNISSYWIHWVRQAPGKGLEWVGY |
| | | | | IDPYSGSTYYADSVKGRFTISADTSK |
| | | | | NTAYLQMNSLRAEDTAVYYCARSDYY |
| | | | | YVDEYMDYWGQGTLVTVSS |
| 2736 | SRP2799-B03 | V_{H} | | MAEVQLVESGGGLVQPGGSLRLSCAA |
| | | | | SGFNISDYWIHWVRQAPGKGLEWVGF |
| | | | | IDPYNGYTNYADSVKGRFTISADTSK |
| | | | | NTAYLQMNSLRAEDTAVYYCARGYGS |
| | | | | WPDYLDYWGQGTLVTVSS |
| 2739 | SRP2799-B06 | V_{H} | | MAEVQLVESGGGLVQPGGSLRLSCAA |
| | | | | SGFNISDYNIHWVRQAPGKGLEWVGY |
| | | | | IDPSNGYTYYADSVKGRFTISADTSK |
| | | | | NTAYLQMNSLRAEDTAVYYCARDDQS |
| | | | | VSSPFDYWGQGTLVTVSS |
| 2751 | SRP2900-A01 | V_{H} | | MEVQLVESGGGLVQPGGSLRLSCAAS |
| | | | | GFTISSYWIHWVRQAPGKGLKWVGYI |
| | | | | DPYNGATYYADSVKGRFTISADTSKN |
| | | | | TAYLQMNSLRAEDTAVYYCARADYYY |
| | | | | VDEYFDYWGQGTLVTVSS |
| 2752 | SRP2900-A02 | V_{H} | | MEVQLVESGGGLVQPGGSLRLSCAAS |
| | | | | GFTISGYIIHWVRQAPGKGLEWVGYI |
| | | | | DPSNGYTYYADSVKGRFTISADTSKN |
| | | | | TAYLQMNSLRAEDTAVYYCARDDQSL |
| | | | | SSPFDYWGQGTLVTVSS |
| 2753 | SRP2900-A03 | V_{H} | | MEVQLVESGGGLVQPGGSLRLSCAAS |
| | | | | GFTIPSYWIHWVRQAPGKGLEWVGYI |
| | | | | DPANGATYYADSVKGRFTISADTSKN |
| | | | | TAYLQMNSLRAEDTAVYYCARADYFY |
| | | | | VDEYLDYWGQGTLVTVSS |
| 2754 | SRP2900-A04 | V_{H} | | MEVQLVESGGGLVQPGGSLRLSCAAS |
| | | | | GFTISGYWIHWVRQAPGKGLEWVGYI |
| | | | | DPYNGATYYADSVKGRFTISADTSKN |
| | | | | AAYLQMNSLRAEDTAVYYCARADYFY |
| | | | | IDEYWDYWGQGTLVTVSS |
| 2755 | SRP2900-A05 | V_{H} | | MEVQLVESGGGLVQPGGSLRLSCAAS |
| | | | | GFTIPDYWIHWVRQAPGKGLEWVGFI |
| | | | | DPYNGYTNYADPVKSRFTISADTSKN |
| | | | | TAYLQMNSLRAEDTAVYYCARADYYY |
| | | | | VDEYWDYWGQGTLVTVSS |
| 2756 | SRP2900-A06 | V_{H} | | MEVQLVESGGGLVQPGGSLRLSCAAS |
| | | | | GFTISSYWIHWVRQAPGKGLEWVGYI |
| | | | | DPFNGATYYADSVKGRFTISADTSKN |
| | | | | TAYLQMNSLRAEDTAVYYCARADYYY |
| | | | | VDEYWDYWGQGTLVTVSS |
| 2757 | SRP2900-A07 | V_{H} | | MEVQLVESGGGLVQPGGSLRLSCAAS |
| | | | | GFTISSYWIHWVRQAPGKGLEWVGYI |
| | | | | DPYNGATYYADSVKGRFTISADTSKN |
| | | | | TAYLQMNSLRAEDTAVYYCARSDYYY |
| | | | | VDEFFDYWGQGTLVTVSS |
| 2758 | SRP2900-A08 | V_{H} | | MEVQLVESGGGLVQPGGSLRLSCAAS |
| | | | | GFTLIGYWIHWVRQAPGKGLEWVGYI |
| | | | | DPYNGATYYADSVKGRFTISADTSKN |
| | | | | TAYLQMNSLRAEDTAVYYCARADYYY |
| | | | | VDEYWDYWGQGTLVTVSS |
| 2759 | SRP2900-A09 | V_{H} | | MEVQLVESGGGLVQPGGSLRLSCAAS |
| | | | | GFTIADYWIHWVRQAPGKGLEWVGYI |
| | | | | DPYNGATYYADSVKGRFTISADTSKN |
| | | | | TAYLQMNSLRAEDTAVYYCARADYYY |
| | | | | VDEYWDYWGQGTLVTVSS |
| 2760 | SRP2900-A10 | V_{H} | | MEVQLVESGGGLVQPGGALRLSCAAS |
| | | | | GFTISSYWIHWVRQAPGKGLEWVGYI |
| | | | | DPYNGATYYADSVKGRFTISADTSKN |
| | | | | TAYLQMNSLRAEDTAVYYCARADYYY |
| | | | | VDEYLDYWGQGTLVTVSS |
| 2761 | SRP2900-A11 | V_{H} | | MEVQLVESGGGLVQPGGSLRLSCAAS |
| | | | | GFTITDYVIHWVRQAPGKGLEWVGYI |
| | | | | DPSNGYTYYADSVKGRFTISADTSKN |
| | | | | TAYLQMNSLRAEDTAVYYCARDDQSF |
| | | | | TSPFDYWGQGTLVTVSS |
| 2762 | SRP2900-B01 | V_{H} | | MEVQLVESGGGLVQPGGSLRLSCAAS |
| | | | | GFTISGYWIHWVRQAPGKGLEWVGYI |
| | | | | DPYNGATYYADSVKGRFTISADTSKN |
| | | | | TAYLQMNSLRAEDTAVYYCARADYYY |
| | | | | VDEYLDYWGQGTLVTVSS |
| 2763 | SRP2900-B02 | V_{H} | | MEVQLVESGGGSVQPGGSLRLSCAAS |
| | | | | GFTISDYWIHWVRQAPGKGLEWVGYI |
| | | | | DPYNGATYYADSVKGRFTISADTSKN |
| | | | | TAYLQMNSLRAEDTAVYYCARADYYY |
| | | | | VDEYWDYWGQGTLVTVSS |
| 2764 | SRP2900-B03 | V_{H} | | MEVQLVESGGGLVQPGGSLRLSCSAS |
| | | | | GFTIADYWIHWVRQAPGKGLEWVGYI |
| | | | | DPYNGATYYADSVKGRFTISADTSKN |
| | | | | TAYLQMNSLRAEDTAVYYCARADYYY |
| | | | | VDEYWDYWGQGTLVTVSS |
| 2765 | SRP2900-B04 | V_{H} | | MEVQLVESGGGLVQPGGSLRLSCAAS |
| | | | | GFTISEYWIHWVRQAPGKGLEWVGFI |
| | | | | DPYNGYTGYADSVRGRFTISADTSKN |
| | | | | TAYLQMNSLRAEDTAVYYCARADYYY |
| | | | | VDEYWDYWGQGTLVTVSS |
| 2766 | SRP2900-B05 | V_{H} | | MEVQLVESGGGLVQPGGSLRLSCAAS |
| | | | | GFTISGYWIHWVRQAPGKGLEWVGYI |
| | | | | DPYNGATYYADSVKGRFTISADTSKN |
| | | | | TAYLQMNSLRAEDTAVYYCARADYYG |
| | | | | VDEFLDYWGQGTLVTVSS |
| 2767 | SRP2900-B06 | V_{H} | | MEVQLVESGGGLVQPGGSLRLSCAAS |
| | | | | GFTIPSYWIHWVRQAPGKGLEWVGYI |
| | | | | DPYNGATYYADSVKGRFTISADTSKN |
| | | | | TAYLQMNSLRAEDTAVYYCARADYHG |
| | | | | VDEYWDYWGQGTLVTVSS |
| 2768 | SRP2900-B07 | V_{H} | | MEVQLVESGGGLVQPGGSLRLSCAAS |
| | | | | GFTIADYWIHWVRQAPGKGLEWVGFI |
| | | | | DPYNGYTGYADSVKGRFTISADNSKN |
| | | | | TAYLQMNSLRAEDTAVYYCARADYHG |
| | | | | VDEYWDYWGQGTLVTVSS |
| 2769 | SRP2900-B08 | V_{H} | | MEVQLVESGGGLVQPGGSLRLSCAAS |
| | | | | GFTIPSYWIHWVRQAPGKGLEWVGYI |
| | | | | DPYNGYTYYADSVKGRFTISADTSKN |
| | | | | TAYLQMNSLRAEDTAVYYCARADYYY |
| | | | | VDEYFDYWGQGTLVTVSS |
| 2770 | SRP2900-B09 | V_{H} | | MEVQLVESGGGLVQPGGSLRLSCAAS |
| | | | | GFTISQYWIHWVRQAPGKGLEWVGFI |
| | | | | DPYNGYTNYADSVKGRFTISADTSEN |
| | | | | TAYLQMNSLRAEDTAVYYCARADYYY |
| | | | | VDEYWDYWGQGTLVTVSS |
| 2771 | SRP2900-B10 | V_{H} | | MEVQLVESGGGLVQPGGSLRLSCAAS |
| | | | | GFTIPAYWIHWVRQAPGKGLEWVGYI |
| | | | | DPYNGSTYYADSVKGRFTISADTSKN |
| | | | | TAYLQMNSLRAEDTAVYYCARADYYY |
| | | | | VDEYWDYWGQGTLVTVSS |
| 2772 | SRP2900-B11 | V_{H} | | MEVQLVESGGGLVQPGGSLRLSCAAS |
| | | | | GFTIPAYWIHWVRQAPGKGLEWVGYI |
| | | | | DPSNGYTYYADSVKGRFTISADASKN |
| | | | | TAYLQMNSLRAEDTAAYYCARADYHG |
| | | | | VDEYWDYWGQGTLVTVSS |
| 2773 | SRP2900-C01 | V_{H} | | MEVQLVESGGGLVQPGGSLRLSCAAS |
| | | | | GFTIGSYWIHWVRQAPGKGLEWVGYI |
| | | | | DPSNGYTYYADSVKGRFTISADTSKN |
| | | | | TAYLQMNSLRAEDTAVYYCARADYYY |
| | | | | VDEYWDYWGQGTLVTVSS |
| 2774 | SRP2900-C02 | V_{H} | | MEVQLVESGGGLVQPGGSLRLSCAAS |
| | | | | GFTISSYWIHWVRQAPGKGLEWVGYI |
| | | | | DPYNGATYYADSVKGRFTISADTSKN |
| | | | | TAYLQMNSLRAEDTAVYYCARADYYY |
| | | | | VDEYWDYWGQGTLVTVSS |
| 2775 | SRP2900-C03 | V_{H} | | MEVQLVESGGGLVQPGGSLRLSCAAS |
| | | | | GFTISGYWIHWVRQAPGKGLEWVGYI |
| | | | | DPYNGATYYADSVKGRFTISADTSKN |
| | | | | TAYLQMNSLRAEDTAVYYCARADYYY |
| | | | | VDEYWDYWGQGTLVTVSS |
| 2776 | SRP2900-C04 | V_{H} | | MEVQLVESGGGLVQPGGSLRLSCAAS |
| | | | | GFTIVSYWIHWVRQAPGKGLEWVGYI |
| | | | | DPYNGATYYADSVKGRFTISADTSKN |
| | | | | TAYLQMNSLRAEDTAVYYCARADYYY |
| | | | | VDEYWDYWGQGTLVTVSS |
| 2777 | SRP2900-C05 | V_{H} | | MEVQLVESGGGLVQPGGSLRLSCAAS |
| | | | | GFTISGYWIHWVRQAPGKGLEWVGYI |
| | | | | DPYNGATYYADSVKGRFTISADTPKN |
| | | | | TAYLQMNSLRAEDTAVYYCARADYHG |
| | | | | VDEYWDYWGQGTLVTVSS |
| 2778 | SRP2900-C06 | V_{H} | | MEVQLVESGGGLVQPGGSLRLSCAAS |
| | | | | GFTISGYWIHWVRQAPGKGLEWVGYI |
| | | | | DPYNGATFYADSVKGRFTISADTSKN |
| | | | | TAYLQMNSLRAEDTAVYYCARADYYY |
| | | | | VDEYWDYWGQGTLVTVSS |
| 2779 | SRP2900-C07 | V_{H} | | MEVQLVESGGGLVQPGGSLRLSCAAS |
| | | | | GFTISGYWIHWVRQAPGKGLEWVGYI |
| | | | | DPYNGATYYADSVKGRFTISADTSKN |
| | | | | TAYLQMNSLRAEDTAVYYCARADYYY |
| | | | | VDEYFDYWGQGTLVTVSS |
| 2780 | SRP2900-C08 | V_{H} | | MEVQLVESGGGLVQPGGSLRLSCAAS |
| | | | | GFTIPAYWIHWVRQAPGKGLEWVGYI |
| | | | | DPYNGSTYHADSVKGRFTISADTSKN |
| | | | | TAYLQMNSLRAEDTAVYYCARADYYY |
| | | | | VDEYWDYWGQGTLVTVSS |
| 2781 | SRP2900-C09 | V_{H} | | MEVQLVESGGGLVQPGGSLRLSCAAS |
| | | | | GFTISDYIIHWVRQAPGKGLEWVGYI |
| | | | | DPSNGYTYHADSVKGRFTISADTSKN |
| | | | | TAYLQMNSLRAEDTAVYYCARDDQSF |
| | | | | SSPFDYWGQGTLVTVSS |
| 2782 | SRP2900-C10 | V_{H} | | MEVQLVESGGGLVQPGGSLRLSCAAS |
| | | | | GFTISDYWIHWVRQAPGKGLEWVGYI |
| | | | | DPYNGYTYYADSVNGRFTISADTSKN |
| | | | | TAYLQMNSLRAEDTAVYYCARDYGSY |
| | | | | LVSLDYWGQGTLVTVSS |
| 2783 | SRP2900-C11 | V_{H} | | MEVQLVESGGGLVQPGGSLRLSCAAS |
| | | | | GFTISQYWIHWVRQAPGKGLEWVGYI |
| | | | | DPYNGYTYYADSVKGRFTISADTSKN |
| | | | | TAYLQMNSLRAEDTAVYYCARDYGSW |
| | | | | RIRLDYWGQGTLVTVSS |
| 2784 | SRP2900-D01 | V_{H} | | MEVQLVESGGGLVQPGGSLRLSCAAS |
| | | | | GFTIHEYWIHWVRQAPGKGLEWVGYI |
| | | | | DPANGFTYYADSVKGRFTISADTSKN |
| | | | | TAYLQMNSLRAEDTAVYYCARDYGSL |
| | | | | QVFLDYWGQGTLVTVSS |
| 2785 | SRP2900-D02 | V_{H} | | MEVQLVESGGGLVQPGGSLRLSCAAS |
| | | | | GFTISDYWIHWVRQAPGKGLEWVGYI |
| | | | | DPYNGYTYYADSVKGRFTISADTSKN |
| | | | | TAYLQMNSLRAEDTAVYYCARDYGSL |
| | | | | QVFIDHWGQGTLVTVSS |
| 2786 | SRP2900-D03 | V_{H} | | MEVQLVESGGGLVQPGGSLRLSCAAS |
| | | | | GFTISQYWIHWVRQAPGKGLEWVGYI |
| | | | | DPYNGYTYYADSVKGRFTISADTSKN |
| | | | | TAYLQMNSLRAEDTAVYYCARDYGSL |
| | | | | QVQLDYWGQGTLVTVSS |
| 2787 | SRP2900-D04 | V_{H} | | MEVQLVESGGGLVQPGGSLRLSCAAS |
| | | | | GFTISDYWIHWVRQAPGKGLEWVGFI |
| | | | | DPYNGYTYHADSVEGRFTISADTSKN |
| | | | | TAYLQMNSLRAEDTAVYYCARDYGSL |
| | | | | QVQLDYWGQGTLVTVSS |
| 2788 | SRP2900-D05 | V_{H} | | MEVQLVESGGGLVQPGGSLRLSCAAS |
| | | | | GFAIANYWIHWVRQAPGKGLEWVGYI |
| | | | | DPYNGYTYYADSVKGRFTISADTSKN |
| | | | | TAYLQMNSLRAEDTAVYYCARDYGSL |
| | | | | EVSLDYWGQGTLVTVSS |
| 2789 | SRP2900-D06 | V_{H} | | MEVQLVESGGGLVQPGGSLRLSCAAS |
| | | | | GFTISDYWIHWVRQAPGKGLEWVGYI |
| | | | | DPQNGWTYYADSVKGRFTISADTSKN |
| | | | | TAYLQMNSLRAEDTAVYYCARDYGSL |
| | | | | TINLDYWGQGTLVTVSS |
| 2790 | SRP2900-D07 | V_{H} | | MEVQLVESGGGLVQPGGSLRLSCAAS |
| | | | | GFTISDYWIHWVRQAPGKGLEWVGYI |
| | | | | DPSNGYTYYADSVKGRFTISADTSRN |
| | | | | TAYLQMNSLRAEDTAVYYCARDYGSL |
| | | | | EVSLDYWGQGTLVTVSS |
| 2791 | SRP2900-D08 | V_{H} | | MEVQLVESGGGLVQPGGSLRLSCAAS |
| | | | | GFTISDYWIHWVRQAPGKGLEWVGYI |
| | | | | DPYNGYTYYADSVKGRFTISADTSKN |
| | | | | TAYLQMNSLRAEDTAVYYCARDYGSL |
| | | | | EVSLDYWGQGTLVTVSS |
| 2792 | SRP2900-D09 | V_{H} | | MEVQLVESGGGLVQPGGSLRLSCAAS |
| | | | | GFTISGYWIHWVRQAPGKGLEWVGYI |
| | | | | DPANGFTYYADSVKGRFTISADTSKN |
| | | | | TAYLQMNSLRAEDTAVYYCARDYGSI |
| | | | | QVFLDYWGQGTLVTVSS |
| 2793 | SRP2900-D10 | V_{H} | | MEVQLVESGGGLVQPGGSLRLSCAAS |
| | | | | GFTISDYWIHWVRQAPGKGLEWVGFI |
| | | | | DPKNGYTYYADSVKGRFTISAGTSKN |
| | | | | TAYLQMNSLRAEDTAVYYCARDYGSL |
| | | | | QVFIDYWGQGTLVTVSS |
| 2794 | SRP2900-D11 | V_{H} | | MEVQLVESGGGLVQPGGSLRLSCAAS |
| | | | | GFTISDYWIHWVRQAPGKGLEWVGYI |
| | | | | DPYNGYTYYADSVKGRFTISADTSKN |
| | | | | TAYLQMNPLRAEDTAVYYCARDYGSL |
| | | | | QVQLDYWGQGTLVTVSS |
| 2795 | SRP2900-E01 | V_{H} | | MEVQLVESGGGLVQPGGSLRLSCAAS |
| | | | | GFTISDYWIHWVRQAPGKGLEWVGYI |
| | | | | DPSNGYTYYADSVKGRFTISADTSKN |
| | | | | TAYLQMNSLRAEDTAVYYCARDYGSL |
| | | | | QVFIDYWGQGTLVTVSS |
| 2796 | SRP2900-E02 | V_{H} | | MEVQLVESGGGLVQPGGSLRLSCAAS |
| | | | | GFTISDYVIHWVRQAPGKGLEWVGYI |
| | | | | DPSNGYTYYADPVKGRFTISADTSKN |
| | | | | TAYLQMNSLRAEDTAVYYCARDPQSF |
| | | | | YIAFDYWGQGTLVTVSS |
| 2797 | SRP2900-E03 | V_{H} | | MEVQLVESGGGLVQPGGSLRLSCAAS |
| | | | | GFTISDYWIHWVRQAPGKGLEWVGYI |
| | | | | DPQNGWTYYADSVKGRFTISADTSKN |
| | | | | TAYLQMNSLRAEDTAVYYCARDYGSL |
| | | | | EVSLDYWGQGTLVTVSS |
| 2798 | SRP2900-E04 | V_{H} | | MEVQLVESGGGLVQPGGSLRLSCAAS |
| | | | | GFTIPDYWIHWVRQAPGKGLEWVGYI |
| | | | | DPYSGSTFYADSVKGRFTISADTSKN |
| | | | | TAYLQMNSLRAEDTAVYYCARADYYY |
| | | | | VDEYLDYWGQGTLVTVSS |
| 2799 | SRP2900-E05 | V_{H} | | MEVQLVESGGGLVQPGGSLRLSCAAS |
| | | | | GFTISGYWIHWVRQAPGKGLEWVGYI |
| | | | | DPANGFTYYADSVKGRFTISADTSKN |
| | | | | TAYLQMNSLRAEDTAVYYCARDYGSV |
| | | | | RVFLDYWGQGTLVTVSS |
| 2800 | SRP2900-E06 | V_{H} | | MEVQLVESGGGLVQPGGSLRLSCAAS |
| | | | | GFTISGYIIHWVRQAPGKGLEWVGYI |
| | | | | DPSNGYTYHADSVKGRFTISADTSKN |
| | | | | TAYLQMNSLRAEDTAVYYCARDDQSV |
| | | | | SSPFDYWGQGTLVTVSS |
| 2801 | SRP2900-E07 | V_{H} | | MEVQLVESGGGLVQPGGSLRLSCAAS |
| | | | | GFTISQYWIHWVRQAPGKGLEWVGYI |
| | | | | DPYNGYTYYADSVKGRFTISADTSKN |
| | | | | TAYLQMNSLRAEDTAVYYCARDYGSL |
| | | | | RVSLDYWGQGTLVTVSS |
| 2802 | SRP2900-E08 | V_{H} | | MEVQLVESGGGLVQPGGSLRLSCAAS |
| | | | | GFTITDYWIHWVRQAPGKGLEWVGYI |
| | | | | DPYNGYTYYADSVKGRFTISADTSKN |
| | | | | TAYLQMNSLRAEDTAVYYCARDYGSL |
| | | | | QVFIDYWGQGTLVTVSS |
| 2803 | SRP2900-E09 | V_{H} | | MEVQLVESGGGLVQPGGSLRLSCAAS |
| | | | | GFTISQYWIHWVRQAPGKGLEWVGYI |
| | | | | DPYNGYTYYADSVKGRFTISADTSKN |
| | | | | TAHLQMNSLRAEDTAVYYCARDYGSF |
| | | | | RVQLDYWGQGTLVTVSS |
| 2804 | SRP2900-E10 | V_{H} | | MEVQLVESGGGLVQPGGSLRLSCAAS |
| | | | | GFTISDYWIHWVRQAPGKGLEWVGYI |
| | | | | DPANGYTYYADSVKGRFTISADTSKN |
| | | | | TAYLQMNSLRAEDTAVYYCARDYGSL |
| | | | | HVQLDYWGQGTLVTVSS |
| 2805 | SRP2900-E11 | V_{H} | | MEVQLVESGGGLVQPGGSLRLSCAAS |
| | | | | GFTISYYIIHWVRQAPGKGLEWVGYI |
| | | | | DPSNGYTYYADSVKGRFTISADTSKN |
| | | | | TAYLQMNSLRAEDTAVYYCARDVQSV |
| | | | | SGEFDYWGQGTLVTVSS |
| 2806 | SRP2900-F01 | V_{H} | | MEVQLVESGGGLVQPGGSLRLSCAAS |
| | | | | GFTISDYWIHWVRQAPGKGLEWVGYI |
| | | | | DPYNGYTYYADSVRGRFTISADTSKN |
| | | | | TAYLQMNSLRAEDTAVYYCARDYGSL |
| | | | | QVFIDYWGQGTLVTVSS |
| 2807 | SRP2900-F02 | V_{H} | | MEVQLVESGGGLVQPGGSLRLACAAS |
| | | | | GFTIGVYIIHWVRQAPGKGLEWVGYI |
| | | | | DPSNGYTYYADSVKGRFTISADTSKN |
| | | | | TAYLQMNSLRAEDTAVYYCARDDQSV |
| | | | | SSPFDYWGQGTLVTVSS |
| 2808 | SRP2900-F03 | V_{H} | | MEVQLVESGGGLVQPGGSLRLSCAAS |
| | | | | GFTISDYWIHWVRQAPGKGLEWVGYI |
| | | | | DPYNGYTYYADSVKGRFTISAGTSKN |
| | | | | TAYLQMNSLRAEDTAVYYCARDYGSL |
| | | | | QVFIDYWGQGTLVTVSS |
| 2809 | SRP2900-F04 | V_{H} | | MEVQLVESGGGLVQPGGSLRLSCAAS |
| | | | | GFTISDYWIHWVRQAPGKGLEWVGYI |
| | | | | DPYNGFTYYADSVKGRFTISADTSKN |
| | | | | TAYLQMNSLRAEDTAVYYCARDYGSL |
| | | | | QVFIDYWGQGTLVTVSS |
| 2810 | SRP2900-F05 | V_{H} | | MEVQLVESGGGLVQPGGSLRLSCAAS |
| | | | | GFTINDYVIHWVRQAPGKGLEWVGYI |
| | | | | DPSNGYTYYADSVKGRFTISADTSKN |
| | | | | TAYLQMNSLRAEDTAVYYCARDDQSL |
| | | | | SSPFDYWGQGTLVTVSS |
| 2811 | SRP2900-F06 | V_{H} | | MEVQLVESGGGLVQPGGSLRLSCAAS |
| | | | | GFTISIYTIHWVRQAPGKGLEWVGYI |
| | | | | DPSNGYTYYADSVKGRLTISADTSKN |
| | | | | TAYLQMNSLRAEDTAVYYCARDPQSI |
| | | | | SSPFDYWGQGTLVTVSS |
| 2812 | SRP2900-F07 | V_{H} | | MEVQLVESGGGLVQPGGSLRLSCAAS |
| | | | | GFTISDYTIHWVRQAPGKGLEWVGYI |
| | | | | DPSNGYTYHADSVKGRFTISADTSKN |
| | | | | TAYLQMNSLRAEDTAVYYCARDPQSV |
| | | | | SSPFDYWGQGTLVTVSS |
| 2813 | SRP2900-F08 | V_{H} | | MEVQLVESGGGLVQPGGSLRLSCAAS |
| | | | | GFTIAYYIIHWVRQAPGKGLEWVGYI |
| | | | | DPSNGYTYYADSVKGRFTISADTSKN |
| | | | | TAYLQMNSLRAEDTAVYYCARDDQSL |
| | | | | SSPFDYWGQGTLVTVSS |
| 2814 | SRP2900-F09 | V_{H} | | MEVQLVESGGGLVQPGGSLRLSCAAS |
| | | | | GFTIHDYVIHWVRQAPGKGLEWVGYI |
| | | | | DPSNGYTYYADSVKGRFTISADTSKN |
| | | | | TAYLQMNSLRAEDTAVYYCARDDQSI |
| | | | | SSPLDYWGQGTLVTVSS |
| 2815 | SRP2900-F10 | V_{H} | | MEVQLVESGGGLVQPGGSLRLSCAAS |
| | | | | GFTISDYVIHWVRQAPGKGLEWVGYI |
| | | | | DPSNGYTYYADSVKGRFTISADTSKN |
| | | | | TAYLQMNSLRAEDTAVYYCARDPQSL |
| | | | | FYPFDYWGQGTLVTVSS |
| 2816 | SRP2900-F11 | V_{H} | | MEVQLVESGGGLVQPGGSLRLSCAAS |
| | | | | GFTISGYIIHWVRQAPGKGLEWVGYI |
| | | | | DPSNGYTFYADSVKGRFTISADTSKN |
| | | | | TAYLRMNSLRAEDTAVYYCARDDQSL |
| | | | | SSPFDYWGQGTLVTVSS |
| 2817 | SRP2900-G01 | V_{H} | | MEVQLVESGGGLVQPGGSLRLSCAAS |
| | | | | GFTIFDYTIHWVRQAPGKGLEWVGYI |
| | | | | DPSNGYTFYADSVKGRFTISADTSKN |
| | | | | TAYLQVNSLRAEDTAVYYCARDIQSV |
| | | | | SGEFDYWGQGTLVTVSS |
| 2818 | SRP2900-G02 | V_{H} | | MEVQLVESGGDLVRPGGSLRLSCAAS |
| | | | | GFTISYYIIHWVRQAPGKGLEWVGYI |
| | | | | DPSNGYTYYADSVKGRFTISADTSKN |
| | | | | TAYLQMNSLRAEDTAVYYCARDDQSV |
| | | | | SSPFEYWGQGTLVTVSS |
| 2819 | SRP2900-G03 | V_{H} | | MEVQLVESGGGLVQPGGSLRLSCAAS |
| | | | | GFTINNYIIHWVRQAPGKGLEWVGYI |
| | | | | DPSNGYTYYADSVKGRFTISADTSKN |
| | | | | TAYLQMNSLRAEDTAVYYCARDPQSL |
| | | | | SSPFDYWGQGTLVTVSS |
| 2820 | SRP2900-G04 | V_{H} | | MEVQLVESGGGLVQPGGSLRLSCAAS |
| | | | | GFTTRDYVIHWVRQAPGKGLEWVGYI |
| | | | | DPSNGYTYYADSVKGRFTISADTSKN |
| | | | | TAYLQMNSLRAEDTAVYYCARDPQSL |
| | | | | SSPFDYWGQGTLVTVSS |
| 2821 | SRP2900-G05 | V_{H} | | MEVQLVESGGGLVQPGGLLRLSCAAS |
| | | | | GFTIAYYVIHWVRQAPGKGLEWVGYI |
| | | | | DPSNGYTYYADSVKGRFTISADTSKN |
| | | | | TAYLQMNSLRAEDTAVYYCARDDQSL |
| | | | | SSPFDYWGQGTLVTVSS |
| 2822 | SRP2900-G06 | V_{H} | | MEVQLVESGGGLVQPGGSLRLSCAAS |
| | | | | GFTISDYIIHWVRQAPGKGLEWVGYI |
| | | | | DPSNGYTYYADSVKGRFTISADTSKN |
| | | | | TAYLQMNSLRAEDTAVYYCARDDQSL |
| | | | | SSPFDYWGQGTLVTVSS |
| 2823 | SRP2900-G07 | V_{H} | | MEVQLVESGGGLVQPGGSLRLSCAAS |
| | | | | GFTISDYVIHWVRQAPGKGLEWVGYI |
| | | | | DPSNGYTYYADSVKGRFTISADTSKN |
| | | | | TAYLQMNSLRAEDTAVYYCARDPQSL |
| | | | | TSPFDYWGQGTLVTVSS |
| 2824 | SRP2900-G08 | V_{H} | | MEVQLVESGGGLVQLGGSLRLSCAAS |
| | | | | GFTIGGYIIHWVRQAPGKGLEWVGYI |
| | | | | DPSNGYTYYADSVKGRFTISADTSKN |
| | | | | TAYLQMNSLRAEDTAVYYCARDIQSV |
| | | | | SGEFDYWGQGTLVTVSS |
| 2825 | SRP2900-G09 | V_{H} | | MEVQLVESGGGLVQPGGSLRLSCAAS |
| | | | | GFTISDYIIHWVRQAPGKGLEWVGYI |
| | | | | DPSNGYTYYADSVKGRFTISADTSKN |
| | | | | TAYLQMNSLRAEDTAVYYCARDPQSL |
| | | | | TSPFDYWGQGTLVTVSS |
| 2826 | SRP2900-G10 | V_{H} | | MEVQLVESGGGLVQPGGSLRLSCAAS |
| | | | | GFTIPDYIIHWVRQAPGKGLEWVGYI |
| | | | | DPSNGYTYYADSVKGRFTISADTSKN |
| | | | | TAYLQMNSLRAEDTAVYYCARDIQSV |
| | | | | SGEFDYWGQGTLVTVSS |
| 2827 | SRP2900-G11 | V_{H} | | MEVQLVESGGGLVQPGGSSRLSCAAS |
| | | | | GFTIYDYIIHWVRQAPGKGLEWVGYI |
| | | | | DPSNGYTDYADSVKGRFTISADTSKN |
| | | | | TAYLQMNSLRAEDTAVYYCARDIQSV |
| | | | | SGEFDYWGQGTLVTVSS |
| 2828 | SRP2900-H01 | V_{H} | | MEVQLVESGGGLVQPGGSLRLSCAAS |
| | | | | GFTIFDYIIHWVRQAPGKGLEWVGYI |
| | | | | DPSNGYTYYADSVKGRFTISADTSKN |
| | | | | TAYLQMNSLRAEDTAVYYCARDPQSV |
| | | | | GSEFDYWGQGTLVTVSS |
| 2829 | SRP2900-H02 | V_{H} | | MEVQLVESGGGLVQPGGSLRLSCAAS |
| | | | | GFTIFDYIIHWVRQAPGKGLEWVGYI |
| | | | | DPSNGYTYYADSVKGRFTISADTSKN |
| | | | | TAYLQMNSLRAEDTAVYYCARDIQSV |
| | | | | SGEFDYWGQGTLVTVSS |
| 2830 | SRP2900-H03 | V_{H} | | MEVQLVESGGGLVQPGGSLRLSCAAS |
| | | | | GFTIYDYIIHWVRQAPGKGLEWVGYI |
| | | | | DPSNGYTYHADSVKGRFTISADTSKN |
| | | | | TAYLQMNSLRAEDTAVYYCARDPQSL |
| | | | | SSPFDYWGQGTLVTVSS |
| 2831 | SRP2900-H04 | V_{H} | | MEVQLVESGGGLVQPGGSLRLSCAAS |
| | | | | GFTISHYIIHWVRQAPGKGLEWVGYI |
| | | | | DPSNGYTYYADSVKGRFTISADTSKN |
| | | | | TAYLQMNSLRAEDTAVYYCARDIQSV |
| | | | | SGEFDYWGQGTLVTVSS |
| 2832 | SRP2900-H05 | V_{H} | | MEVQLVESGGGLVQPGGSLRLSCAAS |
| | | | | GFTISDYTIHWVRQAPGKGLEWVGYI |
| | | | | DPSNGYTYYADSVKGRFTISADTSKN |
| | | | | TAYLQMNSLRAEDTAVYYCARDIQSV |
| | | | | SGEFDYWGQGTLVTVSS |
| 2833 | SRP2900-H06 | V_{H} | | MEVQLVESGGGLVQPGGSLRLSCAAS |
| | | | | GFTIAAYIIHWVRQAPGKGLEWVGYI |
| | | | | DPSNGYTYYADSVKGRFTISADTSKN |
| | | | | TAYLQMNSLRAEDTAVYYCARDDQSF |
| | | | | SSPFDYWGQGTLVTVSS |
| 2834 | SRP2900-H07 | V_{H} | | MEVQLVESGGGLVQPGGSLRLSCAAS |
| | | | | GFNIAYYTIHWVRQAPGKGLEWVGYI |
| | | | | DPSNGYTYYADSVKGRFTISADTSKN |
| | | | | TAYLQMNSLRAEDTAVYYCARDPQSF |
| | | | | SSPFDYWGQGTLVTVSS |
| 2835 | SRP2900-H08 | V_{H} | | MEVQLVESGGGLVQPGGSLRLSCAAS |
| | | | | GFTISDYVIHWVRQAPGKGLEWVGYI |
| | | | | DPSNGYTFYADSVKGRFTISADTPKN |
| | | | | TAYLQMNSLRAKDTAVYYCARDPQSL |
| | | | | SSPFDYWGQGTLVTVSS |
| 2836 | SRP2900-H09 | V_{H} | | MEVQLVESGGGLVQPGGSLRLSCAAS |
| | | | | GFTISDYVIHWVRQAPGKGLEWVGYI |
| | | | | DPSNGYTYYADSVKGRFTISADTSKN |
| | | | | TAYLQMNSLRAEDTAVYYCARDDQSI |
| | | | | SSPLDYWGQGTLVTVSS |
| 2837 | SRP2900-H10 | V_{H} | | MEVQLVESGGGLVQPGGSLRLSCAAS |
| | | | | GFTISDYVIHWVRQAPGKGLEWVGYI |
| | | | | DPSNGFTYYADSVKGRFTISADTSKN |
| | | | | TAYLQTNSLRAEDTAVYYCARDIQSV |
| | | | | SGEFDYWGQGTLVTVSS |
| 2838 | SRP2900-H11 | V_{H} | | MEVQLVESGGGLVQPGGSLRLSCAAS |
| | | | | GFTISSYIIHWVRQAPGKGPEWVGYI |
| | | | | DPSNGYTYYADSVKGRFTISADTSKN |
| | | | | TAYLQMNSLRAEDTAVYYCARDPQTL |
| | | | | TSPFDYWGQGTLVTVSS |
| 2840 | SRP2842-B01 | V_{H} | | QSVEESEGGLFKPADTLTLTCTASGF |
| | | | | SISSYDMSWVRQAPGNGLEWIGIIGS |
| | | | | NGRTYYPNWAKSRSTITRNTNENTVT |
| | | | | LKMTSLTAADTATYFCARGLYDGTGN |
| | | | | IWGPGTLVTVSS |
| 2846 | SRP2842-G04 | V_{H} | | QSVEESEGGLFKPTDTLTLTCTVSGF |
| | | | | SLSYYGVSWVRQAPGNGLEWIGTIDS |
| | | | | SGRTYYASWANGRFTISNDNAPNTVS |
| | | | | LQVNSLAAADTATYFCARDFYGWNSG |
| | | | | ALDIWGPGTLVTVSS |
| 2851 | SRP2901-B05_ | V_{H} | | MQVQVQESGPGLVKPPGTLSLTCAVS |
| | 2842-G04_HC2 | | | GFSLSYYGVSWVRQPPGKGLEWIGTI |
| | | | | DSSGRTYYNPSLKSRVTISNDNAPNT |
| | | | | VSLKLSSVTAADTAVYYCARDFYGWN |
| | | | | SGALDIWGQGTLVTVSS |
| 2857 | SRP2901-D03_ | V_{H} | | MEVQLVESGGGLVQPGGSLRLSCAAS |
| | 2842-B01_HC-3 | | | GFSISSYDMSWVRQAPGKGLEWIGII |
| | | | | GSNGRTYYPDSVKGRFTISRDNSKNT |
| | | | | VTLQMNSLRAEDTAVYYCARGLYDGT |
| | | | | GNIWGQGTLVTVSS |
| 2859 | SRP2901-E03_ | V_{H} | | MEVQLVESGGGLVQPGGSLRLSCAAS |
| | 2842-B01_HC-3 | | | GFSISSYDMSWVRQAPGKGLEWIGII |
| | | | | GSNGRTYYPDSVKGRFTISRDNSKNT |
| | | | | VTLQMNSLRAEDTAVYYCARGLYDGT |
| | | | | GNIWGQGTLVTVSS |
| 2860 | SRP2901-F01_ | V_{H} | | MQVQVVESGGGLVKPGGSLRLSCAAS |
| | 2842-B01_HC-1 | | | GFSISSYDMSWIRQAPGKGLEWIGII |
| | | | | GSNGRTYYADSVKGRFTISRNTNENT |
| | | | | VYLQMNSLRAEDTAVYYCARGLYDGT |
| | | | | GNIWGQGTLVTVSS |
| 2951 | SRP2842-B01 | V_{L} | | ELVMTQTPSPVSAAVGSTVTISCQAS |
| | | | | QSVYGNNWLSWFQQKPGQPPKLLIYG |
| | | | | ASKLASGVSSRFKGSGSGTQFTLTIS |
| | | | | GVQCDDAATYYCQGTYYSGDWYFAFG |
| | | | | GGTELEIL |
| 2957 | SRP2842-G04 | V_{L} | | ELDLTQTASPVSAAVGGTVTINCQAS |
| | | | | QSVYSNKYLSWYQQKPGQPPKLLIYK |
| | | | | ASTLASGVPSRESGSGFGTEFTLTIS |
| | | | | DVQCDDAATYYCAAAYSDDSDTAFGG |
| | | | | GTKVVVE |
| 2962 | SRP2901-B05_ | V_{L} | | MDLQLTQSPSTLSASVGDRVTITCRA |
| | 2842-G04_LC2 | | | SQSVYSNKYLSWYQQKPGKAPKLLIY |
| | | | | KASTLASGVPSRFSGSGSGTEFTLTI |
| | | | | SSLQPDDFATYYCAAAYSDDSDTAFG |
| | | | | QGTKVEIK |
| 2968 | SRP2901-D03_ | V_{L} | | MDLQMTQSPSSLSASVGDRVTITCRA |
| | 2842-B01_LC1 | | | SQSVYGNNWLSWYQQKPGKAPKLLIY |
| | | | | GASKLASGVPSRFSGSGSGTQFTLTI |
| | | | | SSLQPEDFATYYCQGTYYSGDWYFAF |
| | | | | GQGTKVEIK |
| 2970 | SRP2901-E03_ | V_{L} | | MELVMTQSPPTLSLSPGERVTLSCRA |
| | 2842-B01_LC2 | | | SQSVYGNNWLSWYQQKPGQAPRLLIY |
| | | | | GASKLATSIPARFSGSGSGTQFTLTI |
| | | | | SSLQPEDFAVYYCQGTYYSGDWYFAF |
| | | | | GQGTKVEIK |
| 2971 | SRP2901-F01_ | V_{L} | | MDIQMTQSPSSLSASVGDRVTITCQA |
| | 2842-B01_LC3 | | | SQSVYGNNWLSWYQQKPGKAPKLLIY |
| | | | | GASKLASGVPSRFSGSGSGTDFTLTI |
| | | | | SSLQPEDFATYYCQGTYYSGDWYFAF |
| | | | | GGGTKVEIK |
| 3061 | trastuzumab VL | V_{L} | | DIQMTQSPSSLSASVGDRVTITCRAS |
| | | | | QDVNTAVAWYQQKPGKAPKLLIYSAS |
| | | | | FLYSGVPSRFSGSRSGTDFTLTISSL |
| | | | | QPEDFATYYCQQHYTTPPTFGQGTKV |
| | | | | EIK |
| 3062 | Human IgG1 HC | | | ASTKGPSVFPLAPSSKSTSGGTAALG |
| | Constant | | | CLVKDYFPEPVTVSWNSGALTSGVHT |
| | | | | FPAVLQSSGLYSLSSVVTVPSSSLGT |
| | | | | QTYICNVNHKPSNTKVDKKVEPKSCD |
| | | | | KTHTCPPCPAPELLGGPSVFLFPPKP |
| | | | | KDTLMISRTPEVTCVVVDVSHEDPEV |
| | | | | KFNWYVDGVEVHNAKTKPREEQYNST |
| | | | | YRVVSVLTVLHQDWLNGKEYKCKVSN |
| | | | | KALPAPIEKTISKAKGQPREPQVYTL |
| | | | | PPSREEMTKNQVSLTCLVKGFYPSDI |
| | | | | AVEWESNGQPENNYKTTPPVLDSDGS |
| | | | | FFLYSKLTVDKSRWQQGNVFSCSVMH |
| | | | | EALHNHYTQKSLSLSPGK |
| 3063 | Human IgG LC | | | RTVAAPSVFIFPPSDEQLKSGTASVV |
| | Constant Ckappa | | | CLLNNFYPREAKVQWKVDNALQSGNS |
| | | | | QESVTEQDSKDSTYSLSSTLTLSKAD |
| | | | | YEKHKVYACEVTHQGLSSPVTKSFNR |
| | | | | GEC |
| 3064 | Mouse IgG1 HC | | | AKTTPPSVYPLAPGSAAQTNSMVTLG |
| | Constant | | | CLVKGYFPEPVTVTWNSGSLSSGVHT |
| | | | | FPAVLQSDLYTLSSSVTVPSSTWPSE |
| | | | | TVTCNVAHPASSTKVDKKIVPRDCGC |
| | | | | KPCICTVPEVSSVFIFPPKPKDVLTI |
| | | | | TLTPKVTCVVVDISKDDPEVQFSWFV |
| | | | | DDVEVHTAQTQPREEQFNSTERSVSE |
| | | | | LPIMHQDWLNGKEFKCRVNSAAFPAP |
| | | | | IEKTISKTKGRPKAPQVYTIPPPKEQ |
| | | | | MAKDKVSLTCMITDFFPEDITVEWQW |
| | | | | NGQPAENYKNTQPIMDTDGSYFVYSK |
| | | | | LNVQKSNWEAGNTFTCSVLHEGLHNH |
| | | | | HTEKSLSHSPG |
| 3065 | Mouse IgG LC | | | RADAAPTVSIFPPSSEQLTSGGASVV |
| | Constant Ckappa | | | CFLNNFYPKDINVKWKIDGSERQNGV |
| | | | | LNSWTDQDSKDSTYSMSSTLTLTKDE |
| | | | | YERHNSYTCEATHKTSTSPIVKSFNR |
| | | | | NEC |
| 3066 | Kappa LC | | | HMTVAAPSVFIFPPSDEQLKSGTASV |
| | | | | VCLLNNFYPREAKVQWKVDNALQSGN |
| | | | | SQESVTEQDSKDSTYSLSSTLTLSKA |
| | | | | DYEKHKVYACEVTHQGLSSPVTKSFN |
| | | | | RGEC |
| 3067 | Lambda LD | | | GQPKAAPSVTLFPPSSEELQANKATL |
| | | | | VCLISDFYPGAVTVAWKADSSPVKAG |
| | | | | VETTTPSKQSNNKYAASSYLSLTPEQ |
| | | | | WKSHRSYSCQVTHEGSTVEKTVAPTE |
| | | | | CS |
| 3068 | IgG1 Fc from | | | DKTHTCPPCSAPELLGGSSVFLFPPK |
| | scFv-Fc | | | PKDTLMISRTPEVTCVVVDVSHEDPE |
| | | | | VKFNWYVDGVEVHNAKTKPREEQYNS |
| | | | | TYRVVSVLTVLHQDWLNGKEYKCKVS |
| | | | | NKALPAPIEKTISKAKGQPREPQVYT |
| | | | | LPPSRDELTKNQVSLTCLVKGFYPSD |
| | | | | IAVEWESNGQPENNYKTTPPVLDSDG |
| | | | | SFFLYSKLTVDKSRWQQGNVFSCSVM |
| | | | | HEALHNHYTQKSLSLSPGKGSGDYKD |
| | | | | DDDKGSG |
| 3069 | FlagHis Tag | | | GSGDYKDDDDKGSGHHHHHH |
| 3070 | Linker | | | GGGGSGGGGSGGGGS |
| 3071 | Linker | | | AAGSDQEPKSS |
| 3073 | SRP2842-B01 | | | MSKNKQSVEESEGGLFKPADTLTLTC |
| | (scFv (SKNK- | | | TASGFSISSYDMSWVRQAPGNGLEWI |
| | VH-linker-VL)) | | | GIIGSNGRTYYPNWAKSRSTITRNTN |
| | | | | ENTVTLKMTSLTAADTATYFCARGLY |
| | | | | DGTGNIWGPGTLVTVSSGGGGSGGGG |
| | | | | SGGGGSELVMTQTPSPVSAAVGSTVT |
| | | | | ISCQASQSVYGNNWLSWFQQKPGQPP |
| | | | | KLLIYGASKLASGVSSRFKGSGSGTQ |
| | | | | FTLTISGVQCDDAATYYCQGTYYSGD |
| | | | | WYFAFGGGTELEILA |
| 3079 | SRP2842-G04 | | | MSKNKQSVEESEGGLFKPTDTLTLTC |
| | (scFv (SKNK- | | | TVSGFSLSYYGVSWVRQAPGNGLEWI |
| | VH-linker-VL)) | | | GTIDSSGRTYYASWANGRFTISNDNA |
| | | | | PNTVSLQVNSLAAADTATYFCARDFY |
| | | | | GWNSGALDIWGPGTLVTVSSGGGGSG |
| | | | | GGGSGGGGSELDLTQTASPVSAAVGG |
| | | | | TVTINCQASQSVYSNKYLSWYQQKPG |
| | | | | QPPKLLIYKASTLASGVPSRFSGSGF |
| | | | | GTEFTLTISDVQCDDAATYYCAAAYS |
| | | | | DDSDTAFGGGTKVVVEA |
| 3160 | Murine Tissue | | | MAILVRPRLLAALAPTFLGCLLLQVIAGAGI |
| | Factor | | | PEKAFNLTWISTDFKTILEWQPKPTNYTYTV |
| | | | | QISDRSRNWKNKCFSTTDTECDLTDEIVKDV |
| | | | | TWAYEAKVLSVPRRNSVHGDGDQLVIHGEEP |
| | | | | PFTNAPKFLPYRDTNLGQPVIQQFEQDGRKL |
| | | | | NVVVKDSLTLVRKNGTFLTLRQVFGKDLGYI |
| | | | | ITYRKGSSTGKKTNITNTNEFSIDVEEGVSY |
| | | | | CFFVQAMIFSRKTNQNSPGSSTVCTEQWKSF |
| | | | | LGETLIIVGAVVLLATIFIILLSISLCKRRK |
| | | | | NRAGQKGKNTPSRLA |
| 3161 | Human Tissue | | | METPAWPRVPRPETAVARTLLLGWVFAQVAG |
| | Factor Isoform | | | ASGTTNTVAAYNLTWKSTNFKTILEWEPKPV |
| | 2 Precursor | | | NQVYTVQISTKSGDWKSKCFYTTDTECDLTD |
| | | | | EIVKDVKQTYLARVFSYPAGNVESTGSAGEP |
| | | | | LYENSPEFTPYLETNLGQPTIQSFEQVGTKV |
| | | | | NVTVEDERTLVRRNNTFLSLRDVFGKDLIYT |
| | | | | LYYWKSSSSGKKYSTSLELWYLWSSSLSSSW |
| | | | | LYLYTSVERQEWGRAGRRTPH |
| 3162 | Murine Tissue | | | MAILVRPRLLAALAPTFLGCLLLQVTAGAGI |
| | Factor | | | PEKAFNLTWISTDFKTILEWQPKPTNYTYTV |
| | Precursor | | | QISDRSRNWKNKCFSTTDTECDLTDEIVKDV |
| | | | | TWAYEAKVLSVPRRNSVHGDGDQLVIHGEEP |
| | | | | PFTNAPKFLPYRDTNLGQPVIQQFEQDGRKL |
| | | | | NVVVKDSLTLVRKNGTFLTLRQVFGKDLGYI |
| | | | | ITYRKGSSTGKKTNITNTNEFSIDVEEGVSY |
| | | | | CFFVQAMIFSRKTNQNSPGSSTVCTEQWKSF |
| | | | | LGETLIIVGAVVLLATIFIILLSISLCKRRK |
| | | | | NRAGQKGKNTPSRLA |
| 3163 | aTF 2799-A05 | Heavy | | MEVQLVESGGGLVQPGGSLRLSCAASGF |
| | HC | Chain | | NISSYWIHWVRQAPGKGLEWVGYIDPYS |
| | Y180/F404TAG | | | GSTYYADSVKGRFTISADTSKNTAYLQM |
| | | | | NSLRAEDTAVYYCARSDYYYVDEYMDYW |
| | | | | GQGTLVTVSSASTKGPSVFPLAPSSKST |
| | | | | SGGTAALGCLVKDYFPEPVTVSWNSGAL |
| | | | | TSGVHTFPAVLQSSGL*SLSSVVTVPSS |
| | | | | SLGTQTYICNVNHKPSNTKVDKKVEPKS |
| | | | | CDKTHTCPPCPAPELLGGPSVFLFPPKP |
| | | | | KDTLMISRTPEVTCVVVDVSHEDPEVKF |
| | | | | NWYVDGVEVHNAKTKPREEQYNSTYRVV |
| | | | | SVLTVLHQDWLNGKEYKCKVSNKALPAP |
| | | | | IEKTISKAKGQPREPQVYTLPPSREEMT |
| | | | | KNQVSLTCLVKGFYPSDIAVEWESNGQP |
| | | | | ENNYKTTPPVLDSDGS*FLYSKLTVDKS |
| | | | | RWQQGNVFSCSVMHEALHNHYTQKSLSL |
| | | | | SPGK |
| 3164 | aTF 2799-B03 | Heavy | | MEVQLVESGGGLVQPGGSLRLSCAASGF |
| | HC | Chain | | NISDYWIHWVRQAPGKGLEWVGFIDPYN |
| | Y180/F404TAG | | | GYTNYADSVKGRFTISADTSKNTAYLQM |
| | | | | NSLRAEDTAVYYCARGYGSWPDYLDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3165 | aTF 2799-B06 | Heavy | | MEVQLVESGGGLVQPGGSLRLSCAASGF |
| | HC | Chain | | NISDYNIHWVRQAPGKGLEWVGYIDPSN |
| | Y180/F404TAG | | | GYTYYADSVKGRFTISADTSKNTAYLQM |
| | | | | NSLRAEDTAVYYCARDDQSVSSPFDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3168 | aTF_2900- | Heavy | | MEVQLVESGGGLVQPGGSLRLSCAASGF |
| | A02_HC_Y180/ | Chain | | TISGYIIHWVRQAPGKGLEWVGYIDPSN |
| | F404TAG_SerOpt | | | GYTYYADSVKGRFTISADTSKNTAYLQM |
| | | | | NSLRAEDTAVYYCARDDQSLSSPFDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3169 | aTF_2842- | Heavy | | MEVQLVESGGGLVQPGGSLRLSCAASGF |
| | B01_Hc3_Y180/ | Chain | | SISSYDMSWVRQAPGKGLEWIGIIGSNG |
| | F404TAG | | | RTYYPDSVKGRFTISRDNSKNTVTLQMN |
| | | | | SLRAEDTAVYYCARGLYDGTGNIWGQGT |
| | | | | LVTVSSASTKGPSVFPLAPSSKSTSGGT |
| | | | | AALGCLVKDYFPEPVTVSWNSGALTSGV |
| | | | | HTFPAVLQSSGL*SLSSVVTVPSSSLGT |
| | | | | QTYICNVNHKPSNTKVDKKVEPKSCDKT |
| | | | | HTCPPCPAPELLGGPSVFLFPPKPKDTL |
| | | | | MISRTPEVTCVVVDVSHEDPEVKFNWYV |
| | | | | DGVEVHNAKTKPREEQYNSTYRVVSVLT |
| | | | | VLHQDWLNGKEYKCKVSNKALPAPIEKT |
| | | | | ISKAKGQPREPQVYTLPPSREEMTKNQV |
| | | | | SLTCLVKGFYPSDIAVEWESNGQPENNY |
| | | | | KTTPPVLDSDGS*FLYSKLTVDKSRWQQ |
| | | | | GNVFSCSVMHEALHNHYTQKSLSLSPGK |
| 3170 | aTF_2900- | Heavy | | MEVQLVESGGGLVQPGGSLRLSCAASGF |
| | C11_HC_Y180/ | Chain | | TISQYWIHWVRQAPGKGLEWVGYIDPYN |
| | F404TAG_SerOpt | | | GYTYYADSVKGRFTISADTSKNTAYLQM |
| | | | | NSLRAEDTAVYYCARDYGSWRIRLDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3171 | aTF_2900- | Heavy | | MEVQLVESGGGLVQPGGSLRLSCAASGE |
| | F06_HC_Y180/ | Chain | | TISIYTIHWVRQAPGKGLEWVGYIDPSN |
| | F404TAG_SerOpt | | | GYTYYADSVKGRLTISADTSKNTAYLQM |
| | | | | NSLRAEDTAVYYCARDPQSISSPFDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3172 | aTF_2900- | Heavy | | MEVQLVESGGGLVQPGGSLRLSCAASGF |
| | F09_HC_Y180/ | Chain | | TIHDYVIHWVRQAPGKGLEWVGYIDPSN |
| | F404TAG_SerOpt | | | GYTYYADSVKGRFTISADTSKNTAYLQM |
| | | | | NSLRAEDTAVYYCARDDQSISSPLDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3173 | aTF_2900- | Heavy | | MEVQLVESGGGLVQPGGSLRLSCAASGF |
| | H06_HC_Y180/ | Chain | | TIAAYIIHWVRQAPGKGLEWVGYIDPSN |
| | F404TAG_SerOpt | | | GYTYYADSVKGRFTISADTSKNTAYLQM |
| | | | | NSLRAEDTAVYYCARDDQSFSSPFDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3174 | aTF_2900- | Heavy | | MEVQLVESGGGLVQPGGSLRLSCAASGF |
| | H09_HC_Y180/ | Chain | | TISDYVIHWVRQAPGKGLEWVGYIDPSN |
| | F404TAG_SerOpt | | | GYTYYADSVKGRFTISADTSKNTAYLQM |
| | | | | NSLRAEDTAVYYCARDDQSISSPLDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3175 | aTF_2842- | Heavy | | MQVQVVESGGGLVKPGGSLRLSCAASGE |
| | B01_Hc1_Y180/ | Chain | | SISSYDMSWIRQAPGKGLEWIGIIGSNG |
| | F404TAG | | | RTYYADSVKGRFTISRNTNENTVYLQMN |
| | | | | SLRAEDTAVYYCARGLYDGTGNIWGQGT |
| | | | | LVTVSSASTKGPSVFPLAPSSKSTSGGT |
| | | | | AALGCLVKDYFPEPVTVSWNSGALTSGV |
| | | | | HTFPAVLQSSGL*SLSSVVTVPSSSLGT |
| | | | | QTYICNVNHKPSNTKVDKKVEPKSCDKT |
| | | | | HTCPPCPAPELLGGPSVFLFPPKPKDTL |
| | | | | MISRTPEVTCVVVDVSHEDPEVKFNWYV |
| | | | | DGVEVHNAKTKPREEQYNSTYRVVSVLT |
| | | | | VLHQDWLNGKEYKCKVSNKALPAPIEKT |
| | | | | ISKAKGQPREPQVYTLPPSREEMTKNQV |
| | | | | SLTCLVKGFYPSDIAVEWESNGQPENNY |
| | | | | KTTPPVLDSDGS*FLYSKLTVDKSRWQQ |
| | | | | GNVFSCSVMHEALHNHYTQKSLSLSPGK |
| 3176 | aTF_2842- | Heavy | | MQVQVQESGPGLVKPPGTLSLTCAVSGF |
| | G04_Hc2 | Chain | | SLSYYGVSWVRQPPGKGLEWIGTIDSSG |
| | Y180/F404TAG | | | RTYYNPSLKSRVTISNDNAPNTVSLKLS |
| | | | | SVTAADTAVYYCARDFYGWNSGALDIWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3177 | aTF_2900- | Heavy Chain | | MEVQLVESGGGLVQPGGSLRLSCAASGF |
| | A02_HC_Y180/ | | | TISGYIIHWVRQAPGKGLEWVGYIDPSN |
| | F241/F404TAG_ | | | GYTYYADSVKGRFTISADTSKNTAYLQM |
| | SerOpt | | | NSLRAEDTAVYYCARDDQSLSSPFDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSV*LFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3178 | aTF_2900- | Heavy | | MEVQLVESGGGLVQPGGSLRLSCAASGF |
| | A02_HC_F241/ | Chain | | TISGYIIHWVRQAPGKGLEWVGYIDPSN |
| | F404TAG_SerOpt | | | GYTYYADSVKGRFTISADTSKNTAYLQM |
| | | | | NSLRAEDTAVYYCARDDQSLSSPFDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGLYSLSSVVTVPSSS |
| | | | | LGTQTYICNVNHKPSNTKVDKKVEPKSC |
| | | | | DKTHTCPPCPAPELLGGPSV*LFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3179 | Trastuzumab LC | Light | | MDIQMTQSPSSLSASVGDRVTITCRASQ |
| | SerOpt | Chain | | DVNTAVAWYQQKPGKAPKLLIYSASFLY |
| | | | | SGVPSRFSGSRSGTDFTLTISSLQPEDE |
| | | | | ATYYCQQHYTTPPTFGQGTKVEIKRTVA |
| | | | | APSVFIFPPSDEQLKSGTASVVCLLNNE |
| | | | | YPREAKVQWKVDNALQSGNSQESVTEQD |
| | | | | SKDSTYSLSSTLTLSKADYEKHKVYACE |
| | | | | VTHQGLSSPVTKSFNRGEC |
| 3180 | aTF_2842- | Light | | MELVMTQSPPTLSLSPGERVTLSCRASQ |
| | B01_Lc2 | Chain | | SVYGNNWLSWYQQKPGQAPRLLIYGASK |
| | | | | LATSIPARFSGSGSGTQFTLTISSLQPE |
| | | | | DFAVYYCQGTYYSGDWYFAFGQGTKVEI |
| | | | | KRTVAAPSVFIFPPSDEQLKSGTASVVC |
| | | | | LLNNFYPREAKVQWKVDNALQSGNSQES |
| | | | | VTEQDSKDSTYSLSSTLTLSKADYEKHK |
| | | | | VYACEVTHQGLSSPVTKSFNRGEC |
| 3181 | aTF_2842- | Light | | MDLQMTQSPSSLSASVGDRVTITCRASQ |
| | B01_Lc1 | Chain | | SVYGNNWLSWYQQKPGKAPKLLIYGASK |
| | | | | LASGVPSRFSGSGSGTQFTLTISSLQPE |
| | | | | DEATYYCQGTYYSGDWYFAFGQGTKVEI |
| | | | | KRTVAAPSVFIFPPSDEQLKSGTASVVC |
| | | | | LLNNFYPREAKVQWKVDNALQSGNSQES |
| | | | | VTEQDSKDSTYSLSSTLTLSKADYEKHK |
| | | | | VYACEVTHQGLSSPVTKSFNRGEC |
| 3182 | aTF_2842- | Light | | MDIQMTQSPSSLSASVGDRVTITCQASQ |
| | B01_Lc3 | Chain | | SVYGNNWLSWYQQKPGKAPKLLIYGASK |
| | | | | LASGVPSRFSGSGSGTDFTLTISSLQPE |
| | | | | DFATYYCQGTYYSGDWYFAFGGGTKVEI |
| | | | | KRTVAAPSVFIFPPSDEQLKSGTASVVC |
| | | | | LLNNFYPREAKVQWKVDNALQSGNSQES |
| | | | | VTEQDSKDSTYSLSSTLTLSKADYEKHK |
| | | | | VYACEVTHQGLSSPVTKSFNRGEC |
| 3183 | aTF_2842- | Light | | MDLQLTQSPSTLSASVGDRVTITCRASQ |
| | G04_Lc2 | Chain | | SVYSNKYLSWYQQKPGKAPKLLIYKAST |
| | | | | LASGVPSRFSGSGSGTEFTLTISSLQPD |
| | | | | DFATYYCAAAYSDDSDTAFGQGTKVEIK |
| | | | | RTVAAPSVFIFPPSDEQLKSGTASVVCL |
| | | | | LNNFYPREAKVQWKVDNALQSGNSQESV |
| | | | | TEQDSKDSTYSLSSTLTLSKADYEKHKV |
| | | | | YACEVTHQGLSSPVTKSFNRGEC |
| 3184 | trastuzumab LC | Light | | MDIQMTQSPSSLSASVGDRVTITCRASQ |
| | SerOpt | Chain | | DVNTAVAWYQQKPG*APKLLIYSASFLY |
| | K42TAG/ | | | SGVPSRESGSRSGTDFTLTISSLQPEDE |
| | E161TAG/ | | | ATYYCQQHYTTPPTFGQGTKVEIKRTVA |
| | TCT162AGC | | | APSVEIFPPSDEQLKSGTASVVCLLNNE |
| | | | | YPREAKVQWKVDNALQSGNSQ*SVTEQD |
| | | | | SKDSTYSLSSTLTLSKADYEKHKVYACE |
| | | | | VTHQGLSSPVTKSFNRGEC |
| 3185 | trastuzumab LC | Light | | MDIQMTQSPSSLSASVGDRVTITCRASQ |
| | SerOpt K42TAG | Chain | | DVNTAVAWYQQKPG*APKLLIYSASFLY |
| | | | | SGVPSRESGSRSGTDFTLTISSLQPEDE |
| | | | | ATYYCQQHYTTPPTFGQGTKVEIKRTVA |
| | | | | APSVFIFPPSDEQLKSGTASVVCLLNNE |
| | | | | YPREAKVQWKVDNALQSGNSQESVTEQD |
| | | | | SKDSTYSLSSTLTLSKADYEKHKVYACE |
| | | | | VTHQGLSSPVTKSENRGEC |
| 3186 | aTF_2900- | Heavy | | MEVQLVESGGGLVQPGGSLRLSCAASGF |
| | C11_HC_Y180/ | Chain | | TISQYWIHWVRQAPGKGLEWVGYIDPYN |
| | F404TAG_SerOpt | | | GYTY |
| | | | | YADSVKGRFTISADTSKNTAYLQMNSLR |
| | | | | AEDTAVYYCARDYGSWRIRLDYWGQGTL |
| | | | | VTVS |
| | | | | SASTKGPSVFPLAPSSKSTSGGTAALGC |
| | | | | LVKDYFPEPVTVSWNSGALTSGVHTFPA |
| | | | | VLQSSGL*SLSSVVTVPSSSLGTQTYIC |
| | | | | NVNHKPSNTKVDKKVEPKSCDKTHTCPP |
| | | | | CPAPELLGGPSVFLFPPKPKDTLMISRT |
| | | | | PEVTCVVVDVSHEDPEVKFNWYVDGVEV |
| | | | | HNAKTKPREEQYNSTYRVVSVLTVLHQD |
| | | | | WLNGKEYKCKVSNKALPAPIEKTISKAK |
| | | | | GQPREPQVYTLPPSREEMTKNQVSLTCL |
| | | | | VKGFYPSDIAVEWESNGQPENNYKTTPP |
| | | | | VLDSDGS*FLYSKLTVDKSRWQQGNVES |
| | | | | CSVMHEALHNHYTQKSLSLSPGK |
| 3187 | aTF_2900- | Heavy | | MEVQLVESGGGLVQPGGSLRLSCAASGE |
| | F06_HC_Y180/ | Chain | | TISIYTIHWVRQAPGKGLEWVGYIDPSN |
| | F404TAG_SerOpt | | | GYTYYADSVKGRLTISADTSKNTAYLQM |
| | | | | NSLRAEDTAVYYCARDPQSISSPFDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3188 | aTF_2900- | Heavy | | MEVQLVESGGGLVQPGGSLRLSCAASGE |
| | F09_HC_Y180/ | Chain | | TIHDYVIHWVRQAPGKGLEWVGYIDPSN |
| | F404TAG_SerOpt | | | GYTYYADSVKGRFTISADTSKNTAYLQM |
| | | | | NSLRAEDTAVYYCARDDQSISSPLDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3189 | aTF_2900- | Heavy | | MEVQLVESGGGLVQPGGSLRLSCAASGE |
| | H06_HC_Y180/ | Chain | | TIAAYIIHWVRQAPGKGLEWVGYIDPSN |
| | F404TAG_SerOpt | | | GYTYYADSVKGRFTISADTSKNTAYLQM |
| | | | | NSLRAEDTAVYYCARDDQSFSSPFDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3190 | aTF_2900- | Heavy | | MEVQLVESGGGLVQPGGSLRLSCAASGF |
| | H09_HC_Y180/ | Chain | | TISDYVIHWVRQAPGKGLEWVGYIDPSN |
| | F404TAG_SerOpt | | | GYTYYADSVKGRFTISADTSKNTAYLQM |
| | | | | NSLRAEDTAVYYCARDDQSISSPLDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NNYKTTPPVLDSDGS*FLYSKLTVDKSR |
| | | | | WQQGNVFSCSVMHEALHNHYTQKSLSLS |
| | | | | PGK |
| 3191 | aTF_2900- | Heavy | | MEVQLVESGGGLVQPGGSLRLSCAASGF |
| | A02_HC_Y180/ | Chain | | TISGYIIHWVRQAPGKGLEWVGYIDPSN |
| | F241/Y391/ | | | GYTYYADSVKGRFTISADTSKNTAYLQM |
| | F404TAG | | | NSLRAEDTAVYYCARDDQSLSSPFDYWG |
| | | | | QGTLVTVSSASTKGPSVFPLAPSSKSTS |
| | | | | GGTAALGCLVKDYFPEPVTVSWNSGALT |
| | | | | SGVHTFPAVLQSSGL*SLSSVVTVPSSS |
| | | | | LGTQTYICNVNHKPSNTKVDKKVEPKSC |
| | | | | DKTHTCPPCPAPELLGGPSV*LFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYTLPPSREEMTK |
| | | | | NQVSLTCLVKGFYPSDIAVEWESNGQPE |
| | | | | NN*KTTPPVLDSDGS*FLYSKLTVDKSR |
| | | | | WQQGNVFSCSVMHEALHNHYTQKSLSLS |
| | | | | PGK |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

One or more features from any embodiments described herein or in the figures may be combined with one or more features of any other embodiments described herein or in the figures without departing from the scope of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. An antibody conjugate comprising an antibody that specifically binds to tissue factor (TF) linked site-specifically to at least one payload moiety, wherein the antibody comprises one or more non-natural amino acids, and wherein the antibody comprises three heavy chain CDRs from a V_{H} sequence selected from SEQ ID NOs: 2727, 2736, 2739, 2751-2838, 2840, 2846, 2851, 2857, 2859, and 2860, or variants thereof, and three light chain CDRs from a V_{L} sequence selected from SEQ ID NOs: 2951, 2957, 2962, 2968, 2970, 2971, and 3061, or variants thereof.

2-149. (canceled)

150. A kit comprising the antibody conjugate of claim 1 and instructions for use of the antibody conjugate.

151-152. (canceled)

153. A polynucleotide encoding an antibody of claim 117.

154. A vector comprising the polynucleotide of claim 153.

155. A recombinant host cell comprising the vector of claim 154.

156-158. (canceled)

159. A pharmaceutical composition comprising the antibody conjugate of claim 1 and a pharmaceutically acceptable carrier.

160. A pharmaceutical composition comprising the antibody conjugate of claim 1, (b) one or more additional active agents, and (c) a pharmaceutically acceptable carrier.

161-169. (canceled)

170. A method for reducing cell proliferation in a subject in need thereof, comprising administering to the subject an effective amount of an antibody conjugate of claim 1.

171. A method for treating or preventing a disease or condition in a subject in need thereof, comprising administering to the subject an effective amount of an antibody conjugate of claim 1.

172. A method of diagnosing a disease or condition in a subject in need thereof, comprising administering to the subject an effective amount of an antibody conjugate of claim 1.

173. The method of claim 171, further comprising administering to the subject one or more additional active agents.

174-193. (canceled)

194. A method of diagnosing and treating cancer in subject in need thereof, comprising
(a) detecting the expression of TF in a cell or tissue of the subject to diagnose the cancer; and
(b) administering to the subject an effective amount of a pharmaceutical composition of claim 159.

195. The antibody conjugate of claim 1, wherein the antibody comprises one or more non-natural amino acid selected from the group consisting of: HC—F404, HC—K121, HC—Y180, HC—F241, HC-221, HC—Y391, LC-T22, LC-S7, LC-N152, LC-K42, LC-E161, LC-D170, HC—S136, HC—S25, HC-A40, HC—S119, HC—S190, HC—K222, HC—R19, HC—Y52, and HC—S70, according to the Kabat, Chothia, or EU numbering scheme.

196. The antibody conjugate of claim 1, wherein a residue of the one or more non-natural amino acids is linked to the payload moiety via a linker that is cleavable.

197. The antibody conjugate of claim 1, wherein the one or more non-natural amino acids is selected from the group consisting of p-acetyl-L-phenylalanine, O-methyl-L-tyrosine, an -3-(2-naphthyl) alanine, 3-methyl-phenylalanine, O-4-allyl-L-tyrosine, 4-propyl-L-tyrosine, a tri-O-acetyl-GlcNAcβ-serine, L-Dopa, fluorinated phenylalanine, isopropyl-L-phenylalanine, p-azido-L-phenylalanine, p-azido-methyl-L-phenylalanine, compound 56, p-acyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, and p-propargyloxy-phenylalanine.

198. The antibody conjugate of claim 1, wherein the payload moiety is selected from the group consisting of maytansines, hemiasterlins, amanitins, camptothecins, exatecans, anthracyclines, pyrrolobenzodiazepines, and auristatins.

199. The antibody conjugate of claim 1, wherein the antibody comprises a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512.

200. The antibody conjugate of claim 1, wherein the antibody comprises three heavy chain CDRs and three light chain CDRs from a V_{H}/V_{L} pair, or a variant thereof, selected from the group consisting of SEQ ID NOs: 2727/3061, 2736/3061, 2739/3061, 2751/3061, 2752/3061, 2753/3061, 2754/3061, 2755/3061, 2756/3061, 2757/3061, 2758/3061, 2759/3061, 2760/3061, 2761/3061, 2762/3061, 2763/3061, 2764/3061, 2765/3061, 2766/3061, 2767/3061, 2768/3061, 2769/3061, 2770/3061, 2771/3061, 2772/3061, 2773/3061, 2774/3061, 2775/3061, 2776/3061, 2777/3061, 2778/3061, 2779/3061, 2780/3061, 2781/3061, 2782/3061, 2783/3061, 2784/3061, 2785/3061, 2786/3061, 2787/3061, 2788/3061, 2789/3061, 2790/3061, 2791/3061, 2792/3061, 2793/3061, 2794/3061, 2795/3061, 2796/3061, 2797/3061, 2798/3061, 2799/3061, 2800/3061, 2801/3061, 2802/3061, 2803/3061, 2804/3061, 2805/3061, 2806/3061, 2807/3061, 2808/3061, 2809/3061, 2810/3061, 2811/3061, 2812/3061, 2813/3061, 2814/3061, 2815/3061, 2816/3061, 2817/3061, 2818/3061, 2819/3061, 2820/3061, 2821/3061, 2822/3061, 2823/3061, 2824/3061, 2825/3061, 2826/3061, 2827/3061, 2828/3061, 2829/3061, 2830/3061, 2831/3061, 2832/3061, 2833/3061, 2834/3061, 2835/3061, 2836/3061, 2837/3061, 2838/3061, 2840/2951, 2846/2957, 2851/2962, 2857/2968, 2859/2970, and 2860/2971.

201. The antibody conjugate of claim 1, wherein the antibody comprises a V_{H}/V_{L} pair, or a variant thereof, selected from the group consisting of SEQ ID NOs: 2727/3061, 2736/3061, 2739/3061, 2751/3061, 2752/3061, 2753/3061, 2754/3061, 2755/3061, 2756/3061, 2757/3061, 2758/3061, 2759/3061, 2760/3061, 2761/3061, 2762/3061, 2763/3061, 2764/3061, 2765/3061, 2766/3061, 2767/3061, 2768/3061, 2769/3061, 2770/3061, 2771/3061, 2772/3061, 2773/3061, 2774/3061, 2775/3061, 2776/3061, 2777/3061, 2778/3061, 2779/3061, 2780/3061, 2781/3061, 2782/3061, 2783/3061, 2784/3061, 2785/3061, 2786/3061, 2787/3061, 2788/3061, 2789/3061, 2790/3061, 2791/3061, 2792/3061, 2793/3061, 2794/3061, 2795/3061, 2796/3061, 2797/3061, 2798/3061, 2799/3061, 2800/3061, 2801/3061, 2802/3061, 2803/3061, 2804/3061, 2805/3061, 2806/3061, 2807/3061, 2808/3061, 2809/3061, 2810/3061, 2811/3061, 2812/3061, 2813/3061, 2814/3061, 2815/3061, 2816/3061, 2817/3061, 2818/3061, 2819/3061, 2820/3061, 2821/3061, 2822/3061, 2823/3061, 2824/3061, 2825/3061, 2826/3061, 2827/3061, 2828/3061, 2829/3061, 2830/3061, 2831/3061, 2832/3061, 2833/3061, 2834/3061, 2835/3061, 2836/3061, 2837/3061, 2838/3061, 2840/2951, 2846/2957, 2851/2962, 2857/2968, 2859/2970, and 2860/2971.

202. The antibody conjugate of claim 1, wherein the antibody conjugate comprises:
(a) an antibody comprising a V_{H} comprising: a CDR-H1 comprising at least one of SEQ ID NOs: 130 and 455; a CDR-H2 comprising at least one of SEQ ID NOs: 780 and 1105; and a CDR-H3 comprising at least one of SEQ ID NOs: 1430 and 1755; and a V_{L} comprising: a CDR-L1 comprising SEQ ID NO: 2064; a CDR-L2 comprising SEQ ID NO: 2288; and a CDR-L3 comprising SEQ ID NO: 2512;
(b) para-azidomethylphenylalanine residues at antibody sites HC241, HC404, HC180, and HC391;
(c) the following linker-payload, wherein n2 is 8 and each linker-payload is bonded to one of the para-azidomethylphenylalanine residue side chains: [chemical structure]


SEQUENCE LISTING (3191 sequences)

SEQ ID NO: 1 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 2 - protein, 296 aa, Macaca fascicularis
METPAWPRVPRPETAVARTLLLGWVFAQVAGASGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESREIFYIIGAVVFVVIILVIILAISLHKCKKARVGRSWKENSPLNVA

SEQ ID NO: 3-104 - skipped (102 entries)

SEQ ID NO: 105 - protein, 7 aa, synthetic construct
  REGION 1..7: note = Synthetic
GFNISSY

SEQ ID NO: 106-113 - skipped (8 entries)

SEQ ID NO: 114 - protein, 7 aa, synthetic construct
  REGION 1..7: note = Synthetic
GFNISDY

SEQ ID NO: 115-116 - skipped (2 entries)

SEQ ID NO: 117 - protein, 7 aa, synthetic construct
  REGION 1..7: note = Synthetic
GFNISDY

SEQ ID NO: 118-128 - skipped (11 entries)

SEQ ID NO: 129 - protein, 7 aa, synthetic construct
  REGION 1..7: note = Synthetic
GFTISSY

SEQ ID NO: 130 - protein, 7 aa, synthetic construct
  REGION 1..7: note = Synthetic
GFTISGY

SEQ ID NO: 131 - protein, 7 aa, synthetic construct
  REGION 1..7: note = Synthetic
GFTIPSY

SEQ ID NO: 132 - protein, 7 aa, synthetic construct
  REGION 1..7: note = Synthetic
GFTISGY

SEQ ID NO: 133 - protein, 7 aa, synthetic construct
  REGION 1..7: note = Synthetic
GFTIPDY

SEQ ID NO: 134 - protein, 7 aa, synthetic construct
  REGION 1..7: note = Synthetic
GFTISSY

SEQ ID NO: 135 - protein, 7 aa, synthetic construct
  REGION 1..7: note = Synthetic
GFTISSY

SEQ ID NO: 136 - protein, 7 aa, synthetic construct
  REGION 1..7: note = Synthetic
GFTLTGY

SEQ ID NO: 137 - protein, 7 aa, synthetic construct
  REGION 1..7: note = Synthetic
GFTIADY

SEQ ID NO: 138 - protein, 7 aa, synthetic construct
  REGION 1..7: note = Synthetic
GFTISSY

SEQ ID NO: 139 - protein, 7 aa, synthetic construct
  REGION 1..7: note = Synthetic
GFTITDY

SEQ ID NO: 140 - protein, 7 aa, synthetic construct
  REGION 1..7: note = Synthetic
GFTISGY

SEQ ID NO: 141 - protein, 7 aa, synthetic construct
  REGION 1..7: note = Synthetic
GFTISDY

SEQ ID NO: 142 - protein, 7 aa, synthetic construct
  REGION 1..7: note = Synthetic
GFTIADY

SEQ ID NO: 143 - protein, 7 aa, synthetic construct
  REGION 1..7: note = Synthetic
GFTISEY

SEQ ID NO: 144 - protein, 7 aa, synthetic construct
  REGION 1..7: note = Synthetic
GFTISGY

SEQ ID NO: 145 - protein, 7 aa, synthetic construct
  REGION 1..7: note = Synthetic
GFTIPSY

SEQ ID NO: 146 - protein, 7 aa, synthetic construct
  REGION 1..7: note = Synthetic
GFTIADY

SEQ ID NO: 147 - protein, 7 aa, synthetic construct
  REGION 1..7: note = Synthetic
GFTIPSY

SEQ ID NO: 148 - protein, 7 aa, synthetic construct
  REGION 1..7: note = Synthetic
GFTISQY

SEQ ID NO: 149 - protein, 7 aa, synthetic construct
  REGION 1..7: note = Synthetic
GFTIPAY

SEQ ID NO: 150 - protein, 7 aa, synthetic construct
  REGION 1..7: note = Synthetic
GFTIPAY

SEQ ID NO: 151 - protein, 7 aa, synthetic construct
  REGION 1..7: note = Synthetic
GFTIGSY

SEQ ID NO: 152 - protein, 7 aa, synthetic construct
  REGION 1..7: note = Synthetic
GFTISSY

SEQ ID NO: 153 - protein, 7 aa, synthetic construct
  REGION 1..7: note = Synthetic
GFTISGY

SEQ ID NO: 154 - protein, 7 aa, synthetic construct
  REGION 1..7: note = Synthetic
GFTIVSY

SEQ ID NO: 155 - protein, 7 aa, synthetic construct
  REGION 1..7: note = Synthetic
GFTISGY

SEQ ID NO: 156 - protein, 7 aa, synthetic construct
  REGION 1..7: note = Synthetic
GFTISGY

SEQ ID NO: 157 - protein, 7 aa, synthetic construct
  REGION 1..7: note = Synthetic
GFTISGY

SEQ ID NO: 158 - protein, 7 aa, synthetic construct
  REGION 1..7: note = Synthetic
GFTIPAY

SEQ ID NO: 159 - protein, 7 aa, synthetic construct
  REGION 1..7: note = Synthetic
GFTISDY

SEQ ID NO: 160 - protein, 7 aa, synthetic construct
  REGION 1..7: note = Synthetic
GFTISDY

SEQ ID NO: 161 - protein, 7 aa, synthetic construct
  REGION 1..7: note = Synthetic
GFTISQY

SEQ ID NO: 162 - protein, 7 aa, synthetic construct
  REGION 1..7: note = Synthetic
GFTIHEY

SEQ ID NO: 163 - protein, 7 aa, synthetic construct
  REGION 1..7: note = Synthetic
GFTISDY

SEQ ID NO: 164 - protein, 7 aa, synthetic construct
  REGION 1..7: note = Synthetic
GFTISQY

SEQ ID NO: 165 - protein, 7 aa, synthetic construct
  REGION 1..7: note = Synthetic
GFTISDY

SEQ ID NO: 166 - protein, 7 aa, synthetic construct
  REGION 1..7: note = Synthetic
GFAIANY

SEQ ID NO: 167 - protein, 7 aa, synthetic construct
  REGION 1..7: note = Synthetic
GFTISDY

SEQ ID NO: 168 - protein, 7 aa, synthetic construct
  REGION 1..7: note = Synthetic
GFTISDY

SEQ ID NO: 169 - protein, 7 aa, synthetic construct
  REGION 1..7: note = Synthetic
GFTISDY

SEQ ID NO: 170 - protein, 7 aa, synthetic construct
  REGION 1..7: note = Synthetic
GFTISGY

SEQ ID NO: 171 - protein, 7 aa, synthetic construct
  REGION 1..7: note = Synthetic
GFTISDY

SEQ ID NO: 172 - protein, 7 aa, synthetic construct
  REGION 1..7: note = Synthetic
GFTISDY

SEQ ID NO: 173 - protein, 7 aa, synthetic construct
  REGION 1..7: note = Synthetic
GFTISDY

SEQ ID NO: 174 - protein, 7 aa, synthetic construct
  REGION 1..7: note = Synthetic
GFTISDY

SEQ ID NO: 175 - protein, 7 aa, synthetic construct
  REGION 1..7: note = Synthetic
GFTISDY

SEQ ID NO: 176 - protein, 7 aa, synthetic construct
  REGION 1..7: note = Synthetic
GFTIPDY

SEQ ID NO: 177 - protein, 7 aa, synthetic construct
  REGION 1..7: note = Synthetic
GFTISGY

SEQ ID NO: 178 - protein, 7 aa, synthetic construct
  REGION 1..7: note = Synthetic
GFTISGY

SEQ ID NO: 179 - protein, 7 aa, synthetic construct
  REGION 1..7: note = Synthetic
GFTISQY

SEQ ID NO: 180 - protein, 7 aa, synthetic construct
  REGION 1..7: note = Synthetic
GFTITDY

SEQ ID NO: 181 - protein, 7 aa, synthetic construct
  REGION 1..7: note = Synthetic
GFTISQY

SEQ ID NO: 182 - protein, 7 aa, synthetic construct
  REGION 1..7: note = Synthetic
GFTISDY

SEQ ID NO: 183 - protein, 7 aa, synthetic construct
  REGION 1..7: note = Synthetic
GFTISYY

SEQ ID NO: 184 - protein, 7 aa, synthetic construct
  REGION 1..7: note = Synthetic
GFTISDY

SEQ ID NO: 185 - protein, 7 aa, synthetic construct
  REGION 1..7: note = Synthetic
GFTIGVY

SEQ ID NO: 186 - protein, 7 aa, synthetic construct
  REGION 1..7: note = Synthetic
GFTISDY

SEQ ID NO: 187 - protein, 7 aa, synthetic construct
  REGION 1..7: note = Synthetic
GFTISDY

SEQ ID NO: 188 - protein, 7 aa, synthetic construct
  REGION 1..7: note = Synthetic
GFTINDY

SEQ ID NO: 189 - protein, 7 aa, synthetic construct
  REGION 1..7: note = Synthetic
GFTISIY

SEQ ID NO: 190 - protein, 7 aa, synthetic construct
  REGION 1..7: note = Synthetic
GFTISDY

SEQ ID NO: 191 - protein, 7 aa, synthetic construct
  REGION 1..7: note = Synthetic
GFTIAYY

SEQ ID NO: 192 - protein, 7 aa, synthetic construct
  REGION 1..7: note = Synthetic
GFTIHDY

SEQ ID NO: 193 - protein, 7 aa, synthetic construct
  REGION 1..7: note = Synthetic
GFTISDY

SEQ ID NO: 194 - protein, 7 aa, synthetic construct
  REGION 1..7: note = Synthetic
GFTISGY

SEQ ID NO: 195 - protein, 7 aa, synthetic construct
  REGION 1..7: note = Synthetic
GFTIFDY

SEQ ID NO: 196 - protein, 7 aa, synthetic construct
  REGION 1..7: note = Synthetic
GFTISYY

SEQ ID NO: 197 - protein, 7 aa, synthetic construct
  REGION 1..7: note = Synthetic
GFTINNY

SEQ ID NO: 198 - protein, 7 aa, synthetic construct
  REGION 1..7: note = Synthetic
GFTTRDY

SEQ ID NO: 199 - protein, 7 aa, synthetic construct
  REGION 1..7: note = Synthetic
GFTIAYY

SEQ ID NO: 200 - protein, 7 aa, synthetic construct
  REGION 1..7: note = Synthetic
GFTISDY

SEQ ID NO: 201 - protein, 7 aa, synthetic construct
  REGION 1..7: note = Synthetic
GFTISDY

SEQ ID NO: 202 - protein, 7 aa, synthetic construct
  REGION 1..7: note = Synthetic
GFTIGGY

SEQ ID NO: 203 - protein, 7 aa, synthetic construct
  REGION 1..7: note = Synthetic
GFTISDY

SEQ ID NO: 204 - protein, 7 aa, synthetic construct
  REGION 1..7: note = Synthetic
GFTIPDY

SEQ ID NO: 205 - protein, 7 aa, synthetic construct
  REGION 1..7: note = Synthetic
GFTIYDY

SEQ ID NO: 206 - protein, 7 aa, synthetic construct
  REGION 1..7: note = Synthetic
GFTIFDY

SEQ ID NO: 207 - protein, 7 aa, synthetic construct
  REGION 1..7: note = Synthetic
GFTIFDY

SEQ ID NO: 208 - protein, 7 aa, synthetic construct
  REGION 1..7: note = Synthetic
GFTIYDY

SEQ ID NO: 209 - protein, 7 aa, synthetic construct
  REGION 1..7: note = Synthetic
GFTISHY

SEQ ID NO: 210 - protein, 7 aa, synthetic construct
  REGION 1..7: note = Synthetic
GFTISDY

SEQ ID NO: 211 - protein, 7 aa, synthetic construct
  REGION 1..7: note = Synthetic
GFTIAAY

SEQ ID NO: 212 - protein, 7 aa, synthetic construct
  REGION 1..7: note = Synthetic
GFNIAYY

SEQ ID NO: 213 - protein, 7 aa, synthetic construct
  REGION 1..7: note = Synthetic
GFTISDY

SEQ ID NO: 214 - protein, 7 aa, synthetic construct
  REGION 1..7: note = Synthetic
GFTISDY

SEQ ID NO: 215 - protein, 7 aa, synthetic construct
  REGION 1..7: note = Synthetic
GFTISDY

SEQ ID NO: 216 - protein, 7 aa, synthetic construct
  REGION 1..7: note = Synthetic
GFTISSY

SEQ ID NO: 217 - skipped

SEQ ID NO: 218 - protein, 7 aa, synthetic construct
  REGION 1..7: note = Synthetic
GFSISSY

SEQ ID NO: 219-223 - skipped (5 entries)

SEQ ID NO: 224 - protein, 7 aa, synthetic construct
  REGION 1..7: note = Synthetic
GFSLSYY

SEQ ID NO: 225-228 - skipped (4 entries)

SEQ ID NO: 229 - protein, 7 aa, synthetic construct
  REGION 1..7: note = Synthetic
GFSLSYY

SEQ ID NO: 230-234 - skipped (5 entries)

SEQ ID NO: 235 - protein, 7 aa, synthetic construct
  REGION 1..7: note = Synthetic
GFSISSY

SEQ ID NO: 236 - skipped

SEQ ID NO: 237 - protein, 7 aa, synthetic construct
  REGION 1..7: note = Synthetic
GFSISSY

SEQ ID NO: 238 - protein, 7 aa, synthetic construct
  REGION 1..7: note = Synthetic
GFSISSY

SEQ ID NO: 239-429 - skipped (191 entries)

SEQ ID NO: 430 - protein, 5 aa, synthetic construct
  REGION 1..5: note = Synthetic
SYWIH

SEQ ID NO: 431-438 - skipped (8 entries)

SEQ ID NO: 439 - protein, 5 aa, synthetic construct
  REGION 1..5: note = Synthetic
DYWIH

SEQ ID NO: 440-441 - skipped (2 entries)

SEQ ID NO: 442 - protein, 5 aa, synthetic construct
  REGION 1..5: note = Synthetic
DYNIH

SEQ ID NO: 443-453 - skipped (11 entries)

SEQ ID NO: 454 - protein, 5 aa, synthetic construct
  REGION 1..5: note = Synthetic
SYWIH

SEQ ID NO: 455 - protein, 5 aa, synthetic construct
  REGION 1..5: note = Synthetic
GYIIH

SEQ ID NO: 456 - protein, 5 aa, synthetic construct
  REGION 1..5: note = Synthetic
SYWIH

SEQ ID NO: 457 - protein, 5 aa, synthetic construct
  REGION 1..5: note = Synthetic
GYWIH

SEQ ID NO: 458 - protein, 5 aa, synthetic construct
  REGION 1..5: note = Synthetic
DYWIH

SEQ ID NO: 459 - protein, 5 aa, synthetic construct
  REGION 1..5: note = Synthetic
SYWIH

SEQ ID NO: 460 - protein, 5 aa, synthetic construct
  REGION 1..5: note = Synthetic
SYWIH

SEQ ID NO: 461 - protein, 5 aa, synthetic construct
  REGION 1..5: note = Synthetic
GYWIH

SEQ ID NO: 462 - protein, 5 aa, synthetic construct
  REGION 1..5: note = Synthetic
DYWIH

SEQ ID NO: 463 - protein, 5 aa, synthetic construct
  REGION 1..5: note = Synthetic
SYWIH

SEQ ID NO: 464 - protein, 5 aa, synthetic construct
  REGION 1..5: note = Synthetic
DYVIH

SEQ ID NO: 465 - protein, 5 aa, synthetic construct
  REGION 1..5: note = Synthetic
GYWIH

SEQ ID NO: 466 - protein, 5 aa, synthetic construct
  REGION 1..5: note = Synthetic
DYWIH

SEQ ID NO: 467 - protein, 5 aa, synthetic construct
  REGION 1..5: note = Synthetic
DYWIH

SEQ ID NO: 468 - protein, 5 aa, synthetic construct
  REGION 1..5: note = Synthetic
EYWIH

SEQ ID NO: 469 - protein, 5 aa, synthetic construct
  REGION 1..5: note = Synthetic
GYWIH

SEQ ID NO: 470 - protein, 5 aa, synthetic construct
  REGION 1..5: note = Synthetic
SYWIH

SEQ ID NO: 471 - protein, 5 aa, synthetic construct
  REGION 1..5: note = Synthetic
DYWIH

SEQ ID NO: 472 - protein, 5 aa, synthetic construct
  REGION 1..5: note = Synthetic
SYWIH

SEQ ID NO: 473 - protein, 5 aa, synthetic construct
  REGION 1..5: note = Synthetic
QYWIH

SEQ ID NO: 474 - protein, 5 aa, synthetic construct
  REGION 1..5: note = Synthetic
AYWIH

SEQ ID NO: 475 - protein, 5 aa, synthetic construct
  REGION 1..5: note = Synthetic
AYWIH

SEQ ID NO: 476 - protein, 5 aa, synthetic construct
  REGION 1..5: note = Synthetic
SYWIH

SEQ ID NO: 477 - protein, 5 aa, synthetic construct
  REGION 1..5: note = Synthetic
SYWIH

SEQ ID NO: 478 - protein, 5 aa, synthetic construct
  REGION 1..5: note = Synthetic
GYWIH

SEQ ID NO: 479 - protein, 5 aa, synthetic construct
  REGION 1..5: note = Synthetic
SYWIH

SEQ ID NO: 480 - protein, 5 aa, synthetic construct
  REGION 1..5: note = Synthetic
GYWIH

SEQ ID NO: 481 - protein, 5 aa, synthetic construct
  REGION 1..5: note = Synthetic
GYWIH

SEQ ID NO: 482 - protein, 5 aa, synthetic construct
  REGION 1..5: note = Synthetic
GYWIH

SEQ ID NO: 483 - protein, 5 aa, synthetic construct
  REGION 1..5: note = Synthetic
AYWIH

SEQ ID NO: 484 - protein, 5 aa, synthetic construct
  REGION 1..5: note = Synthetic
DYIIH

SEQ ID NO: 485 - protein, 5 aa, synthetic construct
  REGION 1..5: note = Synthetic
DYWIH

SEQ ID NO: 486 - protein, 5 aa, synthetic construct
  REGION 1..5: note = Synthetic
QYWIH

SEQ ID NO: 487 - protein, 5 aa, synthetic construct
  REGION 1..5: note = Synthetic
EYWIH

SEQ ID NO: 488 - protein, 5 aa, synthetic construct
  REGION 1..5: note = Synthetic
DYWIH

SEQ ID NO: 489 - protein, 5 aa, synthetic construct
  REGION 1..5: note = Synthetic
QYWIH

SEQ ID NO: 490 - protein, 5 aa, synthetic construct
  REGION 1..5: note = Synthetic
DYWIH

SEQ ID NO: 491 - protein, 5 aa, synthetic construct
  REGION 1..5: note = Synthetic
NYWIH

SEQ ID NO: 492 - protein, 5 aa, synthetic construct
  REGION 1..5: note = Synthetic
DYWIH

SEQ ID NO: 493 - protein, 5 aa, synthetic construct
  REGION 1..5: note = Synthetic
DYWIH

SEQ ID NO: 494 - protein, 5 aa, synthetic construct
  REGION 1..5: note = Synthetic
DYWIH

SEQ ID NO: 495 - protein, 5 aa, synthetic construct
  REGION 1..5: note = Synthetic
GYWIH

SEQ ID NO: 496 - protein, 5 aa, synthetic construct
  REGION 1..5: note = Synthetic
DYWIH

SEQ ID NO: 497 - protein, 5 aa, synthetic construct
  REGION 1..5: note = Synthetic
DYWIH

SEQ ID NO: 498 - protein, 5 aa, synthetic construct
  REGION 1..5: note = Synthetic
DYWIH

SEQ ID NO: 499 - protein, 5 aa, synthetic construct
  REGION 1..5: note = Synthetic
DYVIH

SEQ ID NO: 500 - protein, 5 aa, synthetic construct
  REGION 1..5: note = Synthetic
DYWIH

SEQ ID NO: 501 - protein, 5 aa, synthetic construct
  REGION 1..5: note = Synthetic
DYWIH

SEQ ID NO: 502 - protein, 5 aa, synthetic construct
  REGION 1..5: note = Synthetic
GYWIH

SEQ ID NO: 503 - protein, 5 aa, synthetic construct
  REGION 1..5: note = Synthetic
GYIIH

SEQ ID NO: 504 - protein, 5 aa, synthetic construct
  REGION 1..5: note = Synthetic
QYWIH

SEQ ID NO: 505 - protein, 5 aa, synthetic construct
  REGION 1..5: note = Synthetic
DYWIH

SEQ ID NO: 506 - protein, 5 aa, synthetic construct
  REGION 1..5: note = Synthetic
QYWIH

SEQ ID NO: 507 - protein, 5 aa, synthetic construct
  REGION 1..5: note = Synthetic
DYWIH

SEQ ID NO: 508 - protein, 5 aa, synthetic construct
  REGION 1..5: note = Synthetic
YYIIH

SEQ ID NO: 509 - protein, 5 aa, synthetic construct
  REGION 1..5: note = Synthetic
DYWIH

SEQ ID NO: 510 - protein, 5 aa, synthetic construct
  REGION 1..5: note = Synthetic
VYIIH

SEQ ID NO: 511 - protein, 5 aa, synthetic construct
  REGION 1..5: note = Synthetic
DYWIH

SEQ ID NO: 512 - protein, 5 aa, synthetic construct
  REGION 1..5: note = Synthetic
DYWIH

SEQ ID NO: 513 - protein, 5 aa, synthetic construct
  REGION 1..5: note = Synthetic
DYVIH

SEQ ID NO: 514 - protein, 5 aa, synthetic construct
  REGION 1..5: note = Synthetic
IYTIH

SEQ ID NO: 515 - protein, 5 aa, synthetic construct
  REGION 1..5: note = Synthetic
DYTIH

SEQ ID NO: 516 - protein, 5 aa, synthetic construct
  REGION 1..5: note = Synthetic
YYIIH

SEQ ID NO: 517 - protein, 5 aa, synthetic construct
  REGION 1..5: note = Synthetic
DYVIH

SEQ ID NO: 518 - protein, 5 aa, synthetic construct
  REGION 1..5: note = Synthetic
DYVIH

SEQ ID NO: 519 - protein, 5 aa, synthetic construct
  REGION 1..5: note = Synthetic
GYIIH

SEQ ID NO: 520 - protein, 5 aa, synthetic construct
  REGION 1..5: note = Synthetic
DYTIH

SEQ ID NO: 521 - protein, 5 aa, synthetic construct
  REGION 1..5: note = Synthetic
YYIIH

SEQ ID NO: 522 - protein, 5 aa, synthetic construct
  REGION 1..5: note = Synthetic
NYIIH

SEQ ID NO: 523 - protein, 5 aa, synthetic construct
  REGION 1..5: note = Synthetic
DYVIH

SEQ ID NO: 524 - protein, 5 aa, synthetic construct
  REGION 1..5: note = Synthetic
YYVIH

SEQ ID NO: 525 - protein, 5 aa, synthetic construct
  REGION 1..5: note = Synthetic
DYIIH

SEQ ID NO: 526 - protein, 5 aa, synthetic construct
  REGION 1..5: note = Synthetic
DYVIH

SEQ ID NO: 527 - protein, 5 aa, synthetic construct
  REGION 1..5: note = Synthetic
GYIIH

SEQ ID NO: 528 - protein, 5 aa, synthetic construct
  REGION 1..5: note = Synthetic
DYIIH

SEQ ID NO: 529 - protein, 5 aa, synthetic construct
  REGION 1..5: note = Synthetic
DYIIH

SEQ ID NO: 530 - protein, 5 aa, synthetic construct
  REGION 1..5: note = Synthetic
DYIIH

SEQ ID NO: 531 - protein, 5 aa, synthetic construct
  REGION 1..5: note = Synthetic
DYIIH

SEQ ID NO: 532 - protein, 5 aa, synthetic construct
  REGION 1..5: note = Synthetic
DYIIH

SEQ ID NO: 533 - protein, 5 aa, synthetic construct
  REGION 1..5: note = Synthetic
DYIIH

SEQ ID NO: 534 - protein, 5 aa, synthetic construct
  REGION 1..5: note = Synthetic
HYIIH

SEQ ID NO: 535 - protein, 5 aa, synthetic construct
  REGION 1..5: note = Synthetic
DYTIH

SEQ ID NO: 536 - protein, 5 aa, synthetic construct
  REGION 1..5: note = Synthetic
AYIIH

SEQ ID NO: 537 - protein, 5 aa, synthetic construct
  REGION 1..5: note = Synthetic
YYTIH

SEQ ID NO: 538 - protein, 5 aa, synthetic construct
  REGION 1..5: note = Synthetic
DYVIH

SEQ ID NO: 539 - protein, 5 aa, synthetic construct
  REGION 1..5: note = Synthetic
DYVIH

SEQ ID NO: 540 - protein, 5 aa, synthetic construct
  REGION 1..5: note = Synthetic
DYVIH

SEQ ID NO: 541 - protein, 5 aa, synthetic construct
  REGION 1..5: note = Synthetic
SYIIH

SEQ ID NO: 542 - skipped

SEQ ID NO: 543 - protein, 5 aa, synthetic construct
  REGION 1..5: note = Synthetic
SYDMS

SEQ ID NO: 544-548 - skipped (5 entries)

SEQ ID NO: 549 - protein, 5 aa, synthetic construct
  REGION 1..5: note = Synthetic
YYGVS

SEQ ID NO: 550-553 - skipped (4 entries)

SEQ ID NO: 554 - protein, 5 aa, synthetic construct
  REGION 1..5: note = Synthetic
YYGVS

SEQ ID NO: 555-559 - skipped (5 entries)

SEQ ID NO: 560 - protein, 5 aa, synthetic construct
  REGION 1..5: note = Synthetic
SYDMS

SEQ ID NO: 561 - skipped

SEQ ID NO: 562 - protein, 5 aa, synthetic construct
  REGION 1..5: note = Synthetic
SYDMS

SEQ ID NO: 563 - protein, 5 aa, synthetic construct
  REGION 1..5: note = Synthetic
SYDMS

SEQ ID NO: 564-754 - skipped (191 entries)

SEQ ID NO: 755 - protein, 6 aa, synthetic construct
  REGION 1..6: note = Synthetic
DPYSGS

SEQ ID NO: 756-763 - skipped (8 entries)

SEQ ID NO: 764 - protein, 6 aa, synthetic construct
  REGION 1..6: note = Synthetic
DPYNGY

SEQ ID NO: 765-766 - skipped (2 entries)

SEQ ID NO: 767 - protein, 6 aa, synthetic construct
  REGION 1..6: note = Synthetic
DPSNGY

SEQ ID NO: 768-778 - skipped (11 entries)

SEQ ID NO: 779 - protein, 6 aa, synthetic construct
  REGION 1..6: note = Synthetic
DPYNGA

SEQ ID NO: 780 - protein, 6 aa, synthetic construct
  REGION 1..6: note = Synthetic
DPSNGY

SEQ ID NO: 781 - protein, 6 aa, synthetic construct
  REGION 1..6: note = Synthetic
DPANGA

SEQ ID NO: 782 - protein, 6 aa, synthetic construct
  REGION 1..6: note = Synthetic
DPYNGA

SEQ ID NO: 783 - protein, 6 aa, synthetic construct
  REGION 1..6: note = Synthetic
DPYNGY

SEQ ID NO: 784 - protein, 6 aa, synthetic construct
  REGION 1..6: note = Synthetic
DPFNGA

SEQ ID NO: 785 - protein, 6 aa, synthetic construct
  REGION 1..6: note = Synthetic
DPYNGA

SEQ ID NO: 786 - protein, 6 aa, synthetic construct
  REGION 1..6: note = Synthetic
DPYNGA

SEQ ID NO: 787 - protein, 6 aa, synthetic construct
  REGION 1..6: note = Synthetic
DPYNGA

SEQ ID NO: 788 - protein, 6 aa, synthetic construct
  REGION 1..6: note = Synthetic
DPYNGA

SEQ ID NO: 789 - protein, 6 aa, synthetic construct
  REGION 1..6: note = Synthetic
DPSNGY

SEQ ID NO: 790 - protein, 6 aa, synthetic construct
  REGION 1..6: note = Synthetic
DPYNGA

SEQ ID NO: 791 - protein, 6 aa, synthetic construct
  REGION 1..6: note = Synthetic
DPYNGA

SEQ ID NO: 792 - protein, 6 aa, synthetic construct
  REGION 1..6: note = Synthetic
DPYNGA

SEQ ID NO: 793 - protein, 6 aa, synthetic construct
  REGION 1..6: note = Synthetic
DPYNGY

SEQ ID NO: 794 - protein, 6 aa, synthetic construct
  REGION 1..6: note = Synthetic
DPYNGA

SEQ ID NO: 795 - protein, 6 aa, synthetic construct
  REGION 1..6: note = Synthetic
DPYNGA

SEQ ID NO: 796 - protein, 6 aa, synthetic construct
  REGION 1..6: note = Synthetic
DPYNGY

SEQ ID NO: 797 - protein, 6 aa, synthetic construct
  REGION 1..6: note = Synthetic
DPYNGY

SEQ ID NO: 798 - protein, 6 aa, synthetic construct
  REGION 1..6: note = Synthetic
DPYNGY

SEQ ID NO: 799 - protein, 6 aa, synthetic construct
  REGION 1..6: note = Synthetic
DPYNGS

SEQ ID NO: 800 - protein, 6 aa, synthetic construct
  REGION 1..6: note = Synthetic
DPSNGY

SEQ ID NO: 801 - protein, 6 aa, synthetic construct
  REGION 1..6: note = Synthetic
DPSNGY

SEQ ID NO: 802 - protein, 6 aa, synthetic construct
  REGION 1..6: note = Synthetic
DPYNGA

SEQ ID NO: 803 - protein, 6 aa, synthetic construct
  REGION 1..6: note = Synthetic
DPYNGA

SEQ ID NO: 804 - protein, 6 aa, synthetic construct
  REGION 1..6: note = Synthetic
DPYNGA

SEQ ID NO: 805 - protein, 6 aa, synthetic construct
  REGION 1..6: note = Synthetic
DPYNGA

SEQ ID NO: 806 - protein, 6 aa, synthetic construct
  REGION 1..6: note = Synthetic
DPYNGA

SEQ ID NO: 807 - protein, 6 aa, synthetic construct
  REGION 1..6: note = Synthetic
DPYNGA

SEQ ID NO: 808 - protein, 6 aa, synthetic construct
  REGION 1..6: note = Synthetic
DPYNGS

SEQ ID NO: 809 - protein, 6 aa, synthetic construct
  REGION 1..6: note = Synthetic
DPSNGY

SEQ ID NO: 810 - protein, 6 aa, synthetic construct
  REGION 1..6: note = Synthetic
DPYNGY

SEQ ID NO: 811 - protein, 6 aa, synthetic construct
  REGION 1..6: note = Synthetic
DPYNGY

SEQ ID NO: 812 - protein, 6 aa, synthetic construct
  REGION 1..6: note = Synthetic
DPANGF

SEQ ID NO: 813 - protein, 6 aa, synthetic construct
  REGION 1..6: note = Synthetic
DPYNGY

SEQ ID NO: 814 - protein, 6 aa, synthetic construct
  REGION 1..6: note = Synthetic
DPYNGY

SEQ ID NO: 815 - protein, 6 aa, synthetic construct
  REGION 1..6: note = Synthetic
DPYNGY

SEQ ID NO: 816 - protein, 6 aa, synthetic construct
  REGION 1..6: note = Synthetic
DPYNGY

SEQ ID NO: 817 - protein, 6 aa, synthetic construct
  REGION 1..6: note = Synthetic
DPQNGW

SEQ ID NO: 818 - protein, 6 aa, synthetic construct
  REGION 1..6: note = Synthetic
DPSNGY

SEQ ID NO: 819 - protein, 6 aa, synthetic construct
  REGION 1..6: note = Synthetic
DPYNGY

SEQ ID NO: 820 - protein, 6 aa, synthetic construct
  REGION 1..6: note = Synthetic
DPANGF

SEQ ID NO: 821 - protein, 6 aa, synthetic construct
  REGION 1..6: note = Synthetic
DPKNGY

SEQ ID NO: 822 - protein, 6 aa, synthetic construct
  REGION 1..6: note = Synthetic
DPYNGY

SEQ ID NO: 823 - protein, 6 aa, synthetic construct
  REGION 1..6: note = Synthetic
DPSNGY

SEQ ID NO: 824 - protein, 6 aa, synthetic construct
  REGION 1..6: note = Synthetic
DPSNGY

SEQ ID NO: 825 - protein, 6 aa, synthetic construct
  REGION 1..6: note = Synthetic
DPQNGW

SEQ ID NO: 826 - protein, 6 aa, synthetic construct
  REGION 1..6: note = Synthetic
DPYSGS

SEQ ID NO: 827 - protein, 6 aa, synthetic construct
  REGION 1..6: note = Synthetic
DPANGF

SEQ ID NO: 828 - protein, 6 aa, synthetic construct
  REGION 1..6: note = Synthetic
DPSNGY

SEQ ID NO: 829 - protein, 6 aa, synthetic construct
  REGION 1..6: note = Synthetic
DPYNGY

SEQ ID NO: 830 - protein, 6 aa, synthetic construct
  REGION 1..6: note = Synthetic
DPYNGY

SEQ ID NO: 831 - protein, 6 aa, synthetic construct
  REGION 1..6: note = Synthetic
DPYNGY

SEQ ID NO: 832 - protein, 6 aa, synthetic construct
  REGION 1..6: note = Synthetic
DPANGY

SEQ ID NO: 833 - protein, 6 aa, synthetic construct
  REGION 1..6: note = Synthetic
DPSNGY

SEQ ID NO: 834 - protein, 6 aa, synthetic construct
  REGION 1..6: note = Synthetic
DPYNGY

SEQ ID NO: 835 - protein, 6 aa, synthetic construct
  REGION 1..6: note = Synthetic
DPSNGY

SEQ ID NO: 836 - protein, 6 aa, synthetic construct
  REGION 1..6: note = Synthetic
DPYNGY

SEQ ID NO: 837 - protein, 6 aa, synthetic construct
  REGION 1..6: note = Synthetic
DPYNGF

SEQ ID NO: 838 - protein, 6 aa, synthetic construct
  REGION 1..6: note = Synthetic
DPSNGY

SEQ ID NO: 839 - protein, 6 aa, synthetic construct
  REGION 1..6: note = Synthetic
DPSNGY

SEQ ID NO: 840 - protein, 6 aa, synthetic construct
  REGION 1..6: note = Synthetic
DPSNGY

SEQ ID NO: 841 - protein, 6 aa, synthetic construct
  REGION 1..6: note = Synthetic
DPSNGY

SEQ ID NO: 842 - protein, 6 aa, synthetic construct
  REGION 1..6: note = Synthetic
DPSNGY

SEQ ID NO: 843 - protein, 6 aa, synthetic construct
  REGION 1..6: note = Synthetic
DPSNGY

SEQ ID NO: 844 - protein, 6 aa, synthetic construct
  REGION 1..6: note = Synthetic
DPSNGY

SEQ ID NO: 845 - protein, 6 aa, synthetic construct
  REGION 1..6: note = Synthetic
DPSNGY

SEQ ID NO: 846 - protein, 6 aa, synthetic construct
  REGION 1..6: note = Synthetic
DPSNGY

SEQ ID NO: 847 - protein, 6 aa, synthetic construct
  REGION 1..6: note = Synthetic
DPSNGY

SEQ ID NO: 848 - protein, 6 aa, synthetic construct
  REGION 1..6: note = Synthetic
DPSNGY

SEQ ID NO: 849 - protein, 6 aa, synthetic construct
  REGION 1..6: note = Synthetic
DPSNGY

SEQ ID NO: 850 - protein, 6 aa, synthetic construct
  REGION 1..6: note = Synthetic
DPSNGY

SEQ ID NO: 851 - protein, 6 aa, synthetic construct
  REGION 1..6: note = Synthetic
DPSNGY

SEQ ID NO: 852 - protein, 6 aa, synthetic construct
  REGION 1..6: note = Synthetic
DPSNGY

SEQ ID NO: 853 - protein, 6 aa, synthetic construct
  REGION 1..6: note = Synthetic
DPSNGY

SEQ ID NO: 854 - protein, 6 aa, synthetic construct
  REGION 1..6: note = Synthetic
DPSNGY

SEQ ID NO: 855 - protein, 6 aa, synthetic construct
  REGION 1..6: note = Synthetic
DPSNGY

SEQ ID NO: 856 - protein, 6 aa, synthetic construct
  REGION 1..6: note = Synthetic
DPSNGY

SEQ ID NO: 857 - protein, 6 aa, synthetic construct
  REGION 1..6: note = Synthetic
DPSNGY

SEQ ID NO: 858 - protein, 6 aa, synthetic construct
  REGION 1..6: note = Synthetic
DPSNGY

SEQ ID NO: 859 - protein, 6 aa, synthetic construct
  REGION 1..6: note = Synthetic
DPSNGY

SEQ ID NO: 860 - protein, 6 aa, synthetic construct
  REGION 1..6: note = Synthetic
DPSNGY

SEQ ID NO: 861 - protein, 6 aa, synthetic construct
  REGION 1..6: note = Synthetic
DPSNGY

SEQ ID NO: 862 - protein, 6 aa, synthetic construct
  REGION 1..6: note = Synthetic
DPSNGY

SEQ ID NO: 863 - protein, 6 aa, synthetic construct
  REGION 1..6: note = Synthetic
DPSNGY

SEQ ID NO: 864 - protein, 6 aa, synthetic construct
  REGION 1..6: note = Synthetic
DPSNGY

SEQ ID NO: 865 - protein, 6 aa, synthetic construct
  REGION 1..6: note = Synthetic
DPSNGF

SEQ ID NO: 866 - protein, 6 aa, synthetic construct
  REGION 1..6: note = Synthetic
DPSNGY

SEQ ID NO: 867 - skipped

SEQ ID NO: 868 - protein, 6 aa, synthetic construct
  REGION 1..6: note = Synthetic
IGSNGR

SEQ ID NO: 869-873 - skipped (5 entries)

SEQ ID NO: 874 - protein, 5 aa, synthetic construct
  REGION 1..5: note = Synthetic
DSSGR

SEQ ID NO: 875-878 - skipped (4 entries)

SEQ ID NO: 879 - protein, 5 aa, synthetic construct
  REGION 1..5: note = Synthetic
DSSGR

SEQ ID NO: 880-884 - skipped (5 entries)

SEQ ID NO: 885 - protein, 6 aa, synthetic construct
  REGION 1..6: note = Synthetic
IGSNGR

SEQ ID NO: 886 - skipped

SEQ ID NO: 887 - protein, 6 aa, synthetic construct
  REGION 1..6: note = Synthetic
IGSNGR

SEQ ID NO: 888 - protein, 6 aa, synthetic construct
  REGION 1..6: note = Synthetic
IGSNGR

SEQ ID NO: 889-1079 - skipped (191 entries)

SEQ ID NO: 1080 - protein, 17 aa, synthetic construct
  REGION 1..17: note = Synthetic
YIDPYSGSTYYADSVKG

SEQ ID NO: 1081-1088 - skipped (8 entries)

SEQ ID NO: 1089 - protein, 17 aa, synthetic construct
  REGION 1..17: note = Synthetic
FIDPYNGYTNYADSVKG

SEQ ID NO: 1090-1091 - skipped (2 entries)

SEQ ID NO: 1092 - protein, 17 aa, synthetic construct
  REGION 1..17: note = Synthetic
YIDPSNGYTYYADSVKG

SEQ ID NO: 1093-1103 - skipped (11 entries)

SEQ ID NO: 1104 - protein, 17 aa, synthetic construct
  REGION 1..17: note = Synthetic
YIDPYNGATYYADSVKG

SEQ ID NO: 1105 - protein, 17 aa, synthetic construct
  REGION 1..17: note = Synthetic
YIDPSNGYTYYADSVKG

SEQ ID NO: 1106 - protein, 17 aa, synthetic construct
  REGION 1..17: note = Synthetic
YIDPANGATYYADSVKG

SEQ ID NO: 1107 - protein, 17 aa, synthetic construct
  REGION 1..17: note = Synthetic
YIDPYNGATYYADSVKG

SEQ ID NO: 1108 - protein, 17 aa, synthetic construct
  REGION 1..17: note = Synthetic
FIDPYNGYTNYADPVKS

SEQ ID NO: 1109 - protein, 17 aa, synthetic construct
  REGION 1..17: note = Synthetic
YIDPFNGATYYADSVKG

SEQ ID NO: 1110 - protein, 17 aa, synthetic construct
  REGION 1..17: note = Synthetic
YIDPYNGATYYADSVKG

SEQ ID NO: 1111 - protein, 17 aa, synthetic construct
  REGION 1..17: note = Synthetic
YIDPYNGATYYADSVKG

SEQ ID NO: 1112 - protein, 17 aa, synthetic construct
  REGION 1..17: note = Synthetic
YIDPYNGATYYADSVKG

SEQ ID NO: 1113 - protein, 17 aa, synthetic construct
  REGION 1..17: note = Synthetic
YIDPYNGATYYADSVKG

SEQ ID NO: 1114 - protein, 17 aa, synthetic construct
  REGION 1..17: note = Synthetic
YIDPSNGYTYYADSVKG

SEQ ID NO: 1115 - protein, 17 aa, synthetic construct
  REGION 1..17: note = Synthetic
YIDPYNGATYYADSVKG

SEQ ID NO: 1116 - protein, 17 aa, synthetic construct
  REGION 1..17: note = Synthetic
YIDPYNGATYYADSVKG

SEQ ID NO: 1117 - protein, 17 aa, synthetic construct
  REGION 1..17: note = Synthetic
YIDPYNGATYYADSVKG

SEQ ID NO: 1118 - protein, 17 aa, synthetic construct
  REGION 1..17: note = Synthetic
FIDPYNGYTGYADSVRG

SEQ ID NO: 1119 - protein, 17 aa, synthetic construct
  REGION 1..17: note = Synthetic
YIDPYNGATYYADSVKG

SEQ ID NO: 1120 - protein, 17 aa, synthetic construct
  REGION 1..17: note = Synthetic
YIDPYNGATYYADSVKG

SEQ ID NO: 1121 - protein, 17 aa, synthetic construct
  REGION 1..17: note = Synthetic
FIDPYNGYTGYADSVKG

SEQ ID NO: 1122 - protein, 17 aa, synthetic construct
  REGION 1..17: note = Synthetic
YIDPYNGYTYYADSVKG

SEQ ID NO: 1123 - protein, 17 aa, synthetic construct
  REGION 1..17: note = Synthetic
FIDPYNGYTNYADSVKG

SEQ ID NO: 1124 - protein, 17 aa, synthetic construct
  REGION 1..17: note = Synthetic
YIDPYNGSTYYADSVKG

SEQ ID NO: 1125 - protein, 17 aa, synthetic construct
  REGION 1..17: note = Synthetic
YIDPSNGYTYYADSVKG

SEQ ID NO: 1126 - protein, 17 aa, synthetic construct
  REGION 1..17: note = Synthetic
YIDPSNGYTYYADSVKG

SEQ ID NO: 1127 - protein, 17 aa, synthetic construct
  REGION 1..17: note = Synthetic
YIDPYNGATYYADSVKG

SEQ ID NO: 1128 - protein, 17 aa, synthetic construct
  REGION 1..17: note = Synthetic
YIDPYNGATYYADSVKG

SEQ ID NO: 1129 - protein, 17 aa, synthetic construct
  REGION 1..17: note = Synthetic
YIDPYNGATYYADSVKG

SEQ ID NO: 1130 - protein, 17 aa, synthetic construct
  REGION 1..17: note = Synthetic
YIDPYNGATYYADSVKG

SEQ ID NO: 1131 - protein, 17 aa, synthetic construct
  REGION 1..17: note = Synthetic
YIDPYNGATFYADSVKG

SEQ ID NO: 1132 - protein, 17 aa, synthetic construct
  REGION 1..17: note = Synthetic
YIDPYNGATYYADSVKG

SEQ ID NO: 1133 - protein, 17 aa, synthetic construct
  REGION 1..17: note = Synthetic
YIDPYNGSTYHADSVKG

SEQ ID NO: 1134 - protein, 17 aa, synthetic construct
  REGION 1..17: note = Synthetic
YIDPSNGYTYHADSVKG

SEQ ID NO: 1135 - protein, 17 aa, synthetic construct
  REGION 1..17: note = Synthetic
YIDPYNGYTYYADSVNG

SEQ ID NO: 1136 - protein, 17 aa, synthetic construct
  REGION 1..17: note = Synthetic
YIDPYNGYTYYADSVKG

SEQ ID NO: 1137 - protein, 17 aa, synthetic construct
  REGION 1..17: note = Synthetic
YIDPANGFTYYADSVKG

SEQ ID NO: 1138 - protein, 17 aa, synthetic construct
  REGION 1..17: note = Synthetic
YIDPYNGYTYYADSVKG

SEQ ID NO: 1139 - protein, 17 aa, synthetic construct
  REGION 1..17: note = Synthetic
YIDPYNGYTYYADSVKG

SEQ ID NO: 1140 - protein, 17 aa, synthetic construct
  REGION 1..17: note = Synthetic
FIDPYNGYTYHADSVEG

SEQ ID NO: 1141 - protein, 17 aa, synthetic construct
  REGION 1..17: note = Synthetic
YIDPYNGYTYYADSVKG

SEQ ID NO: 1142 - protein, 17 aa, synthetic construct
  REGION 1..17: note = Synthetic
YIDPQNGWTYYADSVKG

SEQ ID NO: 1143 - protein, 17 aa, synthetic construct
  REGION 1..17: note = Synthetic
YIDPSNGYTYYADSVKG

SEQ ID NO: 1144 - protein, 17 aa, synthetic construct
  REGION 1..17: note = Synthetic
YIDPYNGYTYYADSVKG

SEQ ID NO: 1145 - protein, 17 aa, synthetic construct
  REGION 1..17: note = Synthetic
YIDPANGFTYYADSVKG

SEQ ID NO: 1146 - protein, 17 aa, synthetic construct
  REGION 1..17: note = Synthetic
FIDPKNGYTYYADSVKG

SEQ ID NO: 1147 - protein, 17 aa, synthetic construct
  REGION 1..17: note = Synthetic
YIDPYNGYTYYADSVKG

SEQ ID NO: 1148 - protein, 17 aa, synthetic construct
  REGION 1..17: note = Synthetic
YIDPSNGYTYYADSVKG

SEQ ID NO: 1149 - protein, 17 aa, synthetic construct
  REGION 1..17: note = Synthetic
YIDPSNGYTYYADPVKG

SEQ ID NO: 1150 - protein, 17 aa, synthetic construct
  REGION 1..17: note = Synthetic
YIDPQNGWTYYADSVKG

SEQ ID NO: 1151 - protein, 17 aa, synthetic construct
  REGION 1..17: note = Synthetic
YIDPYSGSTFYADSVKG

SEQ ID NO: 1152 - protein, 17 aa, synthetic construct
  REGION 1..17: note = Synthetic
YIDPANGFTYYADSVKG

SEQ ID NO: 1153 - protein, 17 aa, synthetic construct
  REGION 1..17: note = Synthetic
YIDPSNGYTYHADSVKG

SEQ ID NO: 1154 - protein, 17 aa, synthetic construct
  REGION 1..17: note = Synthetic
YIDPYNGYTYYADSVKG

SEQ ID NO: 1155 - protein, 17 aa, synthetic construct
  REGION 1..17: note = Synthetic
YIDPYNGYTYYADSVKG

SEQ ID NO: 1156 - protein, 17 aa, synthetic construct
  REGION 1..17: note = Synthetic
YIDPYNGYTYYADSVKG

SEQ ID NO: 1157 - protein, 17 aa, synthetic construct
  REGION 1..17: note = Synthetic
YIDPANGYTYYADSVKG

SEQ ID NO: 1158 - protein, 17 aa, synthetic construct
  REGION 1..17: note = Synthetic
YIDPSNGYTYYADSVKG

SEQ ID NO: 1159 - protein, 17 aa, synthetic construct
  REGION 1..17: note = Synthetic
YIDPYNGYTYYADSVRG

SEQ ID NO: 1160 - protein, 17 aa, synthetic construct
  REGION 1..17: note = Synthetic
YIDPSNGYTYYADSVKG

SEQ ID NO: 1161 - protein, 17 aa, synthetic construct
  REGION 1..17: note = Synthetic
YIDPYNGYTYYADSVKG

SEQ ID NO: 1162 - protein, 17 aa, synthetic construct
  REGION 1..17: note = Synthetic
YIDPYNGFTYYADSVKG

SEQ ID NO: 1163 - protein, 17 aa, synthetic construct
  REGION 1..17: note = Synthetic
YIDPSNGYTYYADSVKG

SEQ ID NO: 1164 - protein, 17 aa, synthetic construct
  REGION 1..17: note = Synthetic
YIDPSNGYTYYADSVKG

SEQ ID NO: 1165 - protein, 17 aa, synthetic construct
  REGION 1..17: note = Synthetic
YIDPSNGYTYHADSVKG

SEQ ID NO: 1166 - protein, 17 aa, synthetic construct
  REGION 1..17: note = Synthetic
YIDPSNGYTYYADSVKG

SEQ ID NO: 1167 - protein, 17 aa, synthetic construct
  REGION 1..17: note = Synthetic
YIDPSNGYTYYADSVKG

SEQ ID NO: 1168 - protein, 17 aa, synthetic construct
  REGION 1..17: note = Synthetic
YIDPSNGYTYYADSVKG

SEQ ID NO: 1169 - protein, 17 aa, synthetic construct
  REGION 1..17: note = Synthetic
YIDPSNGYTFYADSVKG

SEQ ID NO: 1170 - protein, 17 aa, synthetic construct
  REGION 1..17: note = Synthetic
YIDPSNGYTFYADSVKG

SEQ ID NO: 1171 - protein, 17 aa, synthetic construct
  REGION 1..17: note = Synthetic
YIDPSNGYTYYADSVKG

SEQ ID NO: 1172 - protein, 17 aa, synthetic construct
  REGION 1..17: note = Synthetic
YIDPSNGYTYYADSVKG

SEQ ID NO: 1173 - protein, 17 aa, synthetic construct
  REGION 1..17: note = Synthetic
YIDPSNGYTYYADSVKG

SEQ ID NO: 1174 - protein, 17 aa, synthetic construct
  REGION 1..17: note = Synthetic
YIDPSNGYTYYADSVKG

SEQ ID NO: 1175 - protein, 17 aa, synthetic construct
  REGION 1..17: note = Synthetic
YIDPSNGYTYYADSVKG

SEQ ID NO: 1176 - protein, 17 aa, synthetic construct
  REGION 1..17: note = Synthetic
YIDPSNGYTYYADSVKG

SEQ ID NO: 1177 - protein, 17 aa, synthetic construct
  REGION 1..17: note = Synthetic
YIDPSNGYTYYADSVKG

SEQ ID NO: 1178 - protein, 17 aa, synthetic construct
  REGION 1..17: note = Synthetic
YIDPSNGYTYYADSVKG

SEQ ID NO: 1179 - protein, 17 aa, synthetic construct
  REGION 1..17: note = Synthetic
YIDPSNGYTYYADSVKG

SEQ ID NO: 1180 - protein, 17 aa, synthetic construct
  REGION 1..17: note = Synthetic
YIDPSNGYTDYADSVKG

SEQ ID NO: 1181 - protein, 17 aa, synthetic construct
  REGION 1..17: note = Synthetic
YIDPSNGYTYYADSVKG

SEQ ID NO: 1182 - protein, 17 aa, synthetic construct
  REGION 1..17: note = Synthetic
YIDPSNGYTYYADSVKG

SEQ ID NO: 1183 - protein, 17 aa, synthetic construct
  REGION 1..17: note = Synthetic
YIDPSNGYTYHADSVKG

SEQ ID NO: 1184 - protein, 17 aa, synthetic construct
  REGION 1..17: note = Synthetic
YIDPSNGYTYYADSVKG

SEQ ID NO: 1185 - protein, 17 aa, synthetic construct
  REGION 1..17: note = Synthetic
YIDPSNGYTYYADSVKG

SEQ ID NO: 1186 - protein, 17 aa, synthetic construct
  REGION 1..17: note = Synthetic
YIDPSNGYTYYADSVKG

SEQ ID NO: 1187 - protein, 17 aa, synthetic construct
  REGION 1..17: note = Synthetic
YIDPSNGYTYYADSVKG

SEQ ID NO: 1188 - protein, 17 aa, synthetic construct
  REGION 1..17: note = Synthetic
YIDPSNGYTFYADSVKG

SEQ ID NO: 1189 - protein, 17 aa, synthetic construct
  REGION 1..17: note = Synthetic
YIDPSNGYTYYADSVKG

SEQ ID NO: 1190 - protein, 17 aa, synthetic construct
  REGION 1..17: note = Synthetic
YIDPSNGFTYYADSVKG

SEQ ID NO: 1191 - protein, 17 aa, synthetic construct
  REGION 1..17: note = Synthetic
YIDPSNGYTYYADSVKG

SEQ ID NO: 1192 - skipped

SEQ ID NO: 1193 - protein, 16 aa, synthetic construct
  REGION 1..16: note = Synthetic
IIGSNGRTYYPNWAKS

SEQ ID NO: 1194-1198 - skipped (5 entries)

SEQ ID NO: 1199 - protein, 16 aa, synthetic construct
  REGION 1..16: note = Synthetic
TIDSSGRTYYASWANG

SEQ ID NO: 1200-1203 - skipped (4 entries)

SEQ ID NO: 1204 - protein, 16 aa, synthetic construct
  REGION 1..16: note = Synthetic
TIDSSGRTYYNPSLKS

SEQ ID NO: 1205-1209 - skipped (5 entries)

SEQ ID NO: 1210 - protein, 16 aa, synthetic construct
  REGION 1..16: note = Synthetic
IIGSNGRTYYPDSVKG

SEQ ID NO: 1211 - skipped

SEQ ID NO: 1212 - protein, 16 aa, synthetic construct
  REGION 1..16: note = Synthetic
IIGSNGRTYYPDSVKG

SEQ ID NO: 1213 - protein, 16 aa, synthetic construct
  REGION 1..16: note = Synthetic
IIGSNGRTYYADSVKG

SEQ ID NO: 1214-1404 - skipped (191 entries)

SEQ ID NO: 1405 - protein, 9 aa, synthetic construct
  REGION 1..9: note = Synthetic
SDYYYVDEY

SEQ ID NO: 1406-1413 - skipped (8 entries)

SEQ ID NO: 1414 - protein, 8 aa, synthetic construct
  REGION 1..8: note = Synthetic
GYGSWPDY

SEQ ID NO: 1415-1416 - skipped (2 entries)

SEQ ID NO: 1417 - protein, 8 aa, synthetic construct
  REGION 1..8: note = Synthetic
DDQSVSSP

SEQ ID NO: 1418-1428 - skipped (11 entries)

SEQ ID NO: 1429 - protein, 12 aa, synthetic construct
  REGION 1..12: note = Synthetic
ADYYYVDEYFDY

SEQ ID NO: 1430 - protein, 11 aa, synthetic construct
  REGION 1..11: note = Synthetic
DDQSLSSPFDY

SEQ ID NO: 1431 - protein, 12 aa, synthetic construct
  REGION 1..12: note = Synthetic
ADYFYVDEYLDY

SEQ ID NO: 1432 - protein, 12 aa, synthetic construct
  REGION 1..12: note = Synthetic
ADYFYIDEYWDY

SEQ ID NO: 1433 - protein, 12 aa, synthetic construct
  REGION 1..12: note = Synthetic
ADYYYVDEYWDY

SEQ ID NO: 1434 - protein, 12 aa, synthetic construct
  REGION 1..12: note = Synthetic
ADYYYVDEYWDY

SEQ ID NO: 1435 - protein, 12 aa, synthetic construct
  REGION 1..12: note = Synthetic
SDYYYVDEFFDY

SEQ ID NO: 1436 - protein, 12 aa, synthetic construct
  REGION 1..12: note = Synthetic
ADYYYVDEYWDY

SEQ ID NO: 1437 - protein, 12 aa, synthetic construct
  REGION 1..12: note = Synthetic
ADYYYVDEYWDY

SEQ ID NO: 1438 - protein, 12 aa, synthetic construct
  REGION 1..12: note = Synthetic
ADYYYVDEYLDY

SEQ ID NO: 1439 - protein, 11 aa, synthetic construct
  REGION 1..11: note = Synthetic
DDQSFTSPFDY

SEQ ID NO: 1440 - protein, 12 aa, synthetic construct
  REGION 1..12: note = Synthetic
ADYYYVDEYLDY

SEQ ID NO: 1441 - protein, 12 aa, synthetic construct
  REGION 1..12: note = Synthetic
ADYYYVDEYWDY

SEQ ID NO: 1442 - protein, 12 aa, synthetic construct
  REGION 1..12: note = Synthetic
ADYYYVDEYWDY

SEQ ID NO: 1443 - protein, 12 aa, synthetic construct
  REGION 1..12: note = Synthetic
ADYYYVDEYWDY

SEQ ID NO: 1444 - protein, 12 aa, synthetic construct
  REGION 1..12: note = Synthetic
ADYYGVDEFLDY

SEQ ID NO: 1445 - protein, 12 aa, synthetic construct
  REGION 1..12: note = Synthetic
ADYHGVDEYWDY

SEQ ID NO: 1446 - protein, 12 aa, synthetic construct
  REGION 1..12: note = Synthetic
ADYHGVDEYWDY

SEQ ID NO: 1447 - protein, 12 aa, synthetic construct
  REGION 1..12: note = Synthetic
ADYYYVDEYFDY

SEQ ID NO: 1448 - protein, 12 aa, synthetic construct
  REGION 1..12: note = Synthetic
ADYYYVDEYWDY

SEQ ID NO: 1449 - protein, 12 aa, synthetic construct
  REGION 1..12: note = Synthetic
ADYYYVDEYWDY

SEQ ID NO: 1450 - protein, 12 aa, synthetic construct
  REGION 1..12: note = Synthetic
ADYHGVDEYWDY

SEQ ID NO: 1451 - protein, 12 aa, synthetic construct
  REGION 1..12: note = Synthetic
ADYYYVDEYWDY

SEQ ID NO: 1452 - protein, 12 aa, synthetic construct
  REGION 1..12: note = Synthetic
ADYYYVDEYWDY

SEQ ID NO: 1453 - protein, 12 aa, synthetic construct
  REGION 1..12: note = Synthetic
ADYYYVDEYWDY

SEQ ID NO: 1454 - protein, 12 aa, synthetic construct
  REGION 1..12: note = Synthetic
ADYYYVDEYWDY

SEQ ID NO: 1455 - protein, 12 aa, synthetic construct
  REGION 1..12: note = Synthetic
ADYHGVDEYWDY

SEQ ID NO: 1456 - protein, 12 aa, synthetic construct
  REGION 1..12: note = Synthetic
ADYYYVDEYWDY

SEQ ID NO: 1457 - protein, 12 aa, synthetic construct
  REGION 1..12: note = Synthetic
ADYYYVDEYFDY

SEQ ID NO: 1458 - protein, 12 aa, synthetic construct
  REGION 1..12: note = Synthetic
ADYYYVDEYWDY

SEQ ID NO: 1459 - protein, 11 aa, synthetic construct
  REGION 1..11: note = Synthetic
DDQSFSSPFDY

SEQ ID NO: 1460 - protein, 11 aa, synthetic construct
  REGION 1..11: note = Synthetic
DYGSYLVSLDY

SEQ ID NO: 1461 - protein, 11 aa, synthetic construct
  REGION 1..11: note = Synthetic
DYGSWRIRLDY

SEQ ID NO: 1462 - protein, 11 aa, synthetic construct
  REGION 1..11: note = Synthetic
DYGSLQVFLDY

SEQ ID NO: 1463 - protein, 11 aa, synthetic construct
  REGION 1..11: note = Synthetic
DYGSLQVFIDH

SEQ ID NO: 1464 - protein, 11 aa, synthetic construct
  REGION 1..11: note = Synthetic
DYGSLQVQLDY

SEQ ID NO: 1465 - protein, 11 aa, synthetic construct
  REGION 1..11: note = Synthetic
DYGSLQVQLDY

SEQ ID NO: 1466 - protein, 11 aa, synthetic construct
  REGION 1..11: note = Synthetic
DYGSLEVSLDY

SEQ ID NO: 1467 - protein, 11 aa, synthetic construct
  REGION 1..11: note = Synthetic
DYGSLTINLDY

SEQ ID NO: 1468 - protein, 11 aa, synthetic construct
  REGION 1..11: note = Synthetic
DYGSLEVSLDY

SEQ ID NO: 1469 - protein, 11 aa, synthetic construct
  REGION 1..11: note = Synthetic
DYGSLEVSLDY

SEQ ID NO: 1470 - protein, 11 aa, synthetic construct
  REGION 1..11: note = Synthetic
DYGSIQVFLDY

SEQ ID NO: 1471 - protein, 11 aa, synthetic construct
  REGION 1..11: note = Synthetic
DYGSLQVFIDY

SEQ ID NO: 1472 - protein, 11 aa, synthetic construct
  REGION 1..11: note = Synthetic
DYGSLQVQLDY

SEQ ID NO: 1473 - protein, 11 aa, synthetic construct
  REGION 1..11: note = Synthetic
DYGSLQVFIDY

SEQ ID NO: 1474 - protein, 11 aa, synthetic construct
  REGION 1..11: note = Synthetic
DPQSFYIAFDY

SEQ ID NO: 1475 - protein, 11 aa, synthetic construct
  REGION 1..11: note = Synthetic
DYGSLEVSLDY

SEQ ID NO: 1476 - protein, 12 aa, synthetic construct
  REGION 1..12: note = Synthetic
ADYYYVDEYLDY

SEQ ID NO: 1477 - protein, 11 aa, synthetic construct
  REGION 1..11: note = Synthetic
DYGSVRVFLDY

SEQ ID NO: 1478 - protein, 11 aa, synthetic construct
  REGION 1..11: note = Synthetic
DDQSVSSPFDY

SEQ ID NO: 1479 - protein, 11 aa, synthetic construct
  REGION 1..11: note = Synthetic
DYGSLRVSLDY

SEQ ID NO: 1480 - protein, 11 aa, synthetic construct
  REGION 1..11: note = Synthetic
DYGSLQVFIDY

SEQ ID NO: 1481 - protein, 11 aa, synthetic construct
  REGION 1..11: note = Synthetic
DYGSFRVQLDY

SEQ ID NO: 1482 - protein, 11 aa, synthetic construct
  REGION 1..11: note = Synthetic
DYGSLHVQLDY

SEQ ID NO: 1483 - protein, 11 aa, synthetic construct
  REGION 1..11: note = Synthetic
DVQSVSGEFDY

SEQ ID NO: 1484 - protein, 11 aa, synthetic construct
  REGION 1..11: note = Synthetic
DYGSLQVFIDY

SEQ ID NO: 1485 - protein, 11 aa, synthetic construct
  REGION 1..11: note = Synthetic
DDQSVSSPFDY

SEQ ID NO: 1486 - protein, 11 aa, synthetic construct
  REGION 1..11: note = Synthetic
DYGSLQVFIDY

SEQ ID NO: 1487 - protein, 11 aa, synthetic construct
  REGION 1..11: note = Synthetic
DYGSLQVFIDY

SEQ ID NO: 1488 - protein, 11 aa, synthetic construct
  REGION 1..11: note = Synthetic
DDQSLSSPFDY

SEQ ID NO: 1489 - protein, 11 aa, synthetic construct
  REGION 1..11: note = Synthetic
DPQSISSPFDY

SEQ ID NO: 1490 - protein, 11 aa, synthetic construct
  REGION 1..11: note = Synthetic
DPQSVSSPFDY

SEQ ID NO: 1491 - protein, 11 aa, synthetic construct
  REGION 1..11: note = Synthetic
DDQSLSSPFDY

SEQ ID NO: 1492 - protein, 11 aa, synthetic construct
  REGION 1..11: note = Synthetic
DDQSISSPLDY

SEQ ID NO: 1493 - protein, 11 aa, synthetic construct
  REGION 1..11: note = Synthetic
DPQSLFYPFDY

SEQ ID NO: 1494 - protein, 11 aa, synthetic construct
  REGION 1..11: note = Synthetic
DDQSLSSPFDY

SEQ ID NO: 1495 - protein, 11 aa, synthetic construct
  REGION 1..11: note = Synthetic
DIQSVSGEFDY

SEQ ID NO: 1496 - protein, 11 aa, synthetic construct
  REGION 1..11: note = Synthetic
DDQSVSSPFEY

SEQ ID NO: 1497 - protein, 11 aa, synthetic construct
  REGION 1..11: note = Synthetic
DPQSLSSPFDY

SEQ ID NO: 1498 - protein, 11 aa, synthetic construct
  REGION 1..11: note = Synthetic
DPQSLSSPFDY

SEQ ID NO: 1499 - protein, 11 aa, synthetic construct
  REGION 1..11: note = Synthetic
DDQSLSSPFDY

SEQ ID NO: 1500 - protein, 11 aa, synthetic construct
  REGION 1..11: note = Synthetic
DDQSLSSPFDY

SEQ ID NO: 1501 - protein, 11 aa, synthetic construct
  REGION 1..11: note = Synthetic
DPQSLTSPFDY

SEQ ID NO: 1502 - protein, 11 aa, synthetic construct
  REGION 1..11: note = Synthetic
DIQSVSGEFDY

SEQ ID NO: 1503 - protein, 11 aa, synthetic construct
  REGION 1..11: note = Synthetic
DPQSLTSPFDY

SEQ ID NO: 1504 - protein, 11 aa, synthetic construct
  REGION 1..11: note = Synthetic
DIQSVSGEFDY

SEQ ID NO: 1505 - protein, 11 aa, synthetic construct
  REGION 1..11: note = Synthetic
DIQSVSGEFDY

SEQ ID NO: 1506 - protein, 11 aa, synthetic construct
  REGION 1..11: note = Synthetic
DPQSVGSEFDY

SEQ ID NO: 1507 - protein, 11 aa, synthetic construct
  REGION 1..11: note = Synthetic
DIQSVSGEFDY

SEQ ID NO: 1508 - protein, 11 aa, synthetic construct
  REGION 1..11: note = Synthetic
DPQSLSSPFDY

SEQ ID NO: 1509 - protein, 11 aa, synthetic construct
  REGION 1..11: note = Synthetic
DIQSVSGEFDY

SEQ ID NO: 1510 - protein, 11 aa, synthetic construct
  REGION 1..11: note = Synthetic
DIQSVSGEFDY

SEQ ID NO: 1511 - protein, 11 aa, synthetic construct
  REGION 1..11: note = Synthetic
DDQSFSSPFDY

SEQ ID NO: 1512 - protein, 11 aa, synthetic construct
  REGION 1..11: note = Synthetic
DPQSFSSPFDY

SEQ ID NO: 1513 - protein, 11 aa, synthetic construct
  REGION 1..11: note = Synthetic
DPQSLSSPFDY

SEQ ID NO: 1514 - protein, 11 aa, synthetic construct
  REGION 1..11: note = Synthetic
DDQSISSPLDY

SEQ ID NO: 1515 - protein, 11 aa, synthetic construct
  REGION 1..11: note = Synthetic
DIQSVSGEFDY

SEQ ID NO: 1516 - protein, 11 aa, synthetic construct
  REGION 1..11: note = Synthetic
DPQTLTSPFDY

SEQ ID NO: 1517 - skipped

SEQ ID NO: 1518 - protein, 9 aa, synthetic construct
  REGION 1..9: note = Synthetic
GLYDGTGNI

SEQ ID NO: 1519-1523 - skipped (5 entries)

SEQ ID NO: 1524 - protein, 12 aa, synthetic construct
  REGION 1..12: note = Synthetic
DFYGWNSGALDI

SEQ ID NO: 1525-1528 - skipped (4 entries)

SEQ ID NO: 1529 - protein, 12 aa, synthetic construct
  REGION 1..12: note = Synthetic
DFYGWNSGALDI

SEQ ID NO: 1530-1534 - skipped (5 entries)

SEQ ID NO: 1535 - protein, 9 aa, synthetic construct
  REGION 1..9: note = Synthetic
GLYDGTGNI

SEQ ID NO: 1536 - skipped

SEQ ID NO: 1537 - protein, 9 aa, synthetic construct
  REGION 1..9: note = Synthetic
GLYDGTGNI

SEQ ID NO: 1538 - protein, 9 aa, synthetic construct
  REGION 1..9: note = Synthetic
GLYDGTGNI

SEQ ID NO: 1539-1729 - skipped (191 entries)

SEQ ID NO: 1730 - protein, 12 aa, synthetic construct
  REGION 1..12: note = Synthetic
SDYYYVDEYMDY

SEQ ID NO: 1731-1738 - skipped (8 entries)

SEQ ID NO: 1739 - protein, 11 aa, synthetic construct
  REGION 1..11: note = Synthetic
GYGSWPDYLDY

SEQ ID NO: 1740-1741 - skipped (2 entries)

SEQ ID NO: 1742 - protein, 11 aa, synthetic construct
  REGION 1..11: note = Synthetic
DDQSVSSPFDY

SEQ ID NO: 1743-1753 - skipped (11 entries)

SEQ ID NO: 1754 - protein, 12 aa, synthetic construct
  REGION 1..12: note = Synthetic
ADYYYVDEYFDY

SEQ ID NO: 1755 - protein, 11 aa, synthetic construct
  REGION 1..11: note = Synthetic
DDQSLSSPFDY

SEQ ID NO: 1756 - protein, 12 aa, synthetic construct
  REGION 1..12: note = Synthetic
ADYFYVDEYLDY

SEQ ID NO: 1757 - protein, 12 aa, synthetic construct
  REGION 1..12: note = Synthetic
ADYFYIDEYWDY

SEQ ID NO: 1758 - protein, 12 aa, synthetic construct
  REGION 1..12: note = Synthetic
ADYYYVDEYWDY

SEQ ID NO: 1759 - protein, 12 aa, synthetic construct
  REGION 1..12: note = Synthetic
ADYYYVDEYWDY

SEQ ID NO: 1760 - protein, 12 aa, synthetic construct
  REGION 1..12: note = Synthetic
SDYYYVDEFFDY

SEQ ID NO: 1761 - protein, 12 aa, synthetic construct
  REGION 1..12: note = Synthetic
ADYYYVDEYWDY

SEQ ID NO: 1762 - protein, 12 aa, synthetic construct
  REGION 1..12: note = Synthetic
ADYYYVDEYWDY

SEQ ID NO: 1763 - protein, 12 aa, synthetic construct
  REGION 1..12: note = Synthetic
ADYYYVDEYLDY

SEQ ID NO: 1764 - protein, 11 aa, synthetic construct
  REGION 1..11: note = Synthetic
DDQSFTSPFDY

SEQ ID NO: 1765 - protein, 12 aa, synthetic construct
  REGION 1..12: note = Synthetic
ADYYYVDEYLDY

SEQ ID NO: 1766 - protein, 12 aa, synthetic construct
  REGION 1..12: note = Synthetic
ADYYYVDEYWDY

SEQ ID NO: 1767 - protein, 12 aa, synthetic construct
  REGION 1..12: note = Synthetic
ADYYYVDEYWDY

SEQ ID NO: 1768 - protein, 12 aa, synthetic construct
  REGION 1..12: note = Synthetic
ADYYYVDEYWDY

SEQ ID NO: 1769 - protein, 12 aa, synthetic construct
  REGION 1..12: note = Synthetic
ADYYGVDEFLDY

SEQ ID NO: 1770 - protein, 12 aa, synthetic construct
  REGION 1..12: note = Synthetic
ADYHGVDEYWDY

SEQ ID NO: 1771 - protein, 12 aa, synthetic construct
  REGION 1..12: note = Synthetic
ADYHGVDEYWDY

SEQ ID NO: 1772 - protein, 12 aa, synthetic construct
  REGION 1..12: note = Synthetic
ADYYYVDEYFDY

SEQ ID NO: 1773 - protein, 12 aa, synthetic construct
  REGION 1..12: note = Synthetic
ADYYYVDEYWDY

SEQ ID NO: 1774 - protein, 12 aa, synthetic construct
  REGION 1..12: note = Synthetic
ADYYYVDEYWDY

SEQ ID NO: 1775 - protein, 12 aa, synthetic construct
  REGION 1..12: note = Synthetic
ADYHGVDEYWDY

SEQ ID NO: 1776 - protein, 12 aa, synthetic construct
  REGION 1..12: note = Synthetic
ADYYYVDEYWDY

SEQ ID NO: 1777 - protein, 12 aa, synthetic construct
  REGION 1..12: note = Synthetic
ADYYYVDEYWDY

SEQ ID NO: 1778 - protein, 12 aa, synthetic construct
  REGION 1..12: note = Synthetic
ADYYYVDEYWDY

SEQ ID NO: 1779 - protein, 12 aa, synthetic construct
  REGION 1..12: note = Synthetic
ADYYYVDEYWDY

SEQ ID NO: 1780 - protein, 12 aa, synthetic construct
  REGION 1..12: note = Synthetic
ADYHGVDEYWDY

SEQ ID NO: 1781 - protein, 12 aa, synthetic construct
  REGION 1..12: note = Synthetic
ADYYYVDEYWDY

SEQ ID NO: 1782 - protein, 12 aa, synthetic construct
  REGION 1..12: note = Synthetic
ADYYYVDEYFDY

SEQ ID NO: 1783 - protein, 12 aa, synthetic construct
  REGION 1..12: note = Synthetic
ADYYYVDEYWDY

SEQ ID NO: 1784 - protein, 11 aa, synthetic construct
  REGION 1..11: note = Synthetic
DDQSFSSPFDY

SEQ ID NO: 1785 - protein, 11 aa, synthetic construct
  REGION 1..11: note = Synthetic
DYGSYLVSLDY

SEQ ID NO: 1786 - protein, 11 aa, synthetic construct
  REGION 1..11: note = Synthetic
DYGSWRIRLDY

SEQ ID NO: 1787 - protein, 11 aa, synthetic construct
  REGION 1..11: note = Synthetic
DYGSLQVFLDY

SEQ ID NO: 1788 - protein, 11 aa, synthetic construct
  REGION 1..11: note = Synthetic
DYGSLQVFIDH

SEQ ID NO: 1789 - protein, 11 aa, synthetic construct
  REGION 1..11: note = Synthetic
DYGSLQVQLDY

SEQ ID NO: 1790 - protein, 11 aa, synthetic construct
  REGION 1..11: note = Synthetic
DYGSLQVQLDY

SEQ ID NO: 1791 - protein, 11 aa, synthetic construct
  REGION 1..11: note = Synthetic
DYGSLEVSLDY

SEQ ID NO: 1792 - protein, 11 aa, synthetic construct
  REGION 1..11: note = Synthetic
DYGSLTINLDY

SEQ ID NO: 1793 - protein, 11 aa, synthetic construct
  REGION 1..11: note = Synthetic
DYGSLEVSLDY

SEQ ID NO: 1794 - protein, 11 aa, synthetic construct
  REGION 1..11: note = Synthetic
DYGSLEVSLDY

SEQ ID NO: 1795 - protein, 11 aa, synthetic construct
  REGION 1..11: note = Synthetic
DYGSIQVFLDY

SEQ ID NO: 1796 - protein, 11 aa, synthetic construct
  REGION 1..11: note = Synthetic
DYGSLQVFIDY

SEQ ID NO: 1797 - protein, 11 aa, synthetic construct
  REGION 1..11: note = Synthetic
DYGSLQVQLDY

SEQ ID NO: 1798 - protein, 11 aa, synthetic construct
  REGION 1..11: note = Synthetic
DYGSLQVFIDY

SEQ ID NO: 1799 - protein, 11 aa, synthetic construct
  REGION 1..11: note = Synthetic
DPQSFYIAFDY

SEQ ID NO: 1800 - protein, 11 aa, synthetic construct
  REGION 1..11: note = Synthetic
DYGSLEVSLDY

SEQ ID NO: 1801 - protein, 12 aa, synthetic construct
  REGION 1..12: note = Synthetic
ADYYYVDEYLDY

SEQ ID NO: 1802 - protein, 11 aa, synthetic construct
  REGION 1..11: note = Synthetic
DYGSVRVFLDY

SEQ ID NO: 1803 - protein, 11 aa, synthetic construct
  REGION 1..11: note = Synthetic
DDQSVSSPFDY

SEQ ID NO: 1804 - protein, 11 aa, synthetic construct
  REGION 1..11: note = Synthetic
DYGSLRVSLDY

SEQ ID NO: 1805 - protein, 11 aa, synthetic construct
  REGION 1..11: note = Synthetic
DYGSLQVFIDY

SEQ ID NO: 1806 - protein, 11 aa, synthetic construct
  REGION 1..11: note = Synthetic
DYGSFRVQLDY

SEQ ID NO: 1807 - protein, 11 aa, synthetic construct
  REGION 1..11: note = Synthetic
DYGSLHVQLDY

SEQ ID NO: 1808 - protein, 11 aa, synthetic construct
  REGION 1..11: note = Synthetic
DVQSVSGEFDY

SEQ ID NO: 1809 - protein, 11 aa, synthetic construct
  REGION 1..11: note = Synthetic
DYGSLQVFIDY

SEQ ID NO: 1810 - protein, 11 aa, synthetic construct
  REGION 1..11: note = Synthetic
DDQSVSSPFDY

SEQ ID NO: 1811 - protein, 11 aa, synthetic construct
  REGION 1..11: note = Synthetic
DYGSLQVFIDY

SEQ ID NO: 1812 - protein, 11 aa, synthetic construct
  REGION 1..11: note = Synthetic
DYGSLQVFIDY

SEQ ID NO: 1813 - protein, 11 aa, synthetic construct
  REGION 1..11: note = Synthetic
DDQSLSSPFDY

SEQ ID NO: 1814 - protein, 11 aa, synthetic construct
  REGION 1..11: note = Synthetic
DPQSISSPFDY

SEQ ID NO: 1815 - protein, 11 aa, synthetic construct
  REGION 1..11: note = Synthetic
DPQSVSSPFDY

SEQ ID NO: 1816 - protein, 11 aa, synthetic construct
  REGION 1..11: note = Synthetic
DDQSLSSPFDY

SEQ ID NO: 1817 - protein, 11 aa, synthetic construct
  REGION 1..11: note = Synthetic
DDQSISSPLDY

SEQ ID NO: 1818 - protein, 11 aa, synthetic construct
  REGION 1..11: note = Synthetic
DPQSLFYPFDY

SEQ ID NO: 1819 - protein, 11 aa, synthetic construct
  REGION 1..11: note = Synthetic
DDQSLSSPFDY

SEQ ID NO: 1820 - protein, 11 aa, synthetic construct
  REGION 1..11: note = Synthetic
DIQSVSGEFDY

SEQ ID NO: 1821 - protein, 11 aa, synthetic construct
  REGION 1..11: note = Synthetic
DDQSVSSPFEY

SEQ ID NO: 1822 - protein, 11 aa, synthetic construct
  REGION 1..11: note = Synthetic
DPQSLSSPFDY

SEQ ID NO: 1823 - protein, 11 aa, synthetic construct
  REGION 1..11: note = Synthetic
DPQSLSSPFDY

SEQ ID NO: 1824 - protein, 11 aa, synthetic construct
  REGION 1..11: note = Synthetic
DDQSLSSPFDY

SEQ ID NO: 1825 - protein, 11 aa, synthetic construct
  REGION 1..11: note = Synthetic
DDQSLSSPFDY

SEQ ID NO: 1826 - protein, 11 aa, synthetic construct
  REGION 1..11: note = Synthetic
DPQSLTSPFDY

SEQ ID NO: 1827 - protein, 11 aa, synthetic construct
  REGION 1..11: note = Synthetic
DIQSVSGEFDY

SEQ ID NO: 1828 - protein, 11 aa, synthetic construct
  REGION 1..11: note = Synthetic
DPQSLTSPFDY

SEQ ID NO: 1829 - protein, 11 aa, synthetic construct
  REGION 1..11: note = Synthetic
DIQSVSGEFDY

SEQ ID NO: 1830 - protein, 11 aa, synthetic construct
  REGION 1..11: note = Synthetic
DIQSVSGEFDY

SEQ ID NO: 1831 - protein, 11 aa, synthetic construct
  REGION 1..11: note = Synthetic
DPQSVGSEFDY

SEQ ID NO: 1832 - protein, 11 aa, synthetic construct
  REGION 1..11: note = Synthetic
DIQSVSGEFDY

SEQ ID NO: 1833 - protein, 11 aa, synthetic construct
  REGION 1..11: note = Synthetic
DPQSLSSPFDY

SEQ ID NO: 1834 - protein, 11 aa, synthetic construct
  REGION 1..11: note = Synthetic
DIQSVSGEFDY

SEQ ID NO: 1835 - protein, 11 aa, synthetic construct
  REGION 1..11: note = Synthetic
DIQSVSGEFDY

SEQ ID NO: 1836 - protein, 11 aa, synthetic construct
  REGION 1..11: note = Synthetic
DDQSFSSPFDY

SEQ ID NO: 1837 - protein, 11 aa, synthetic construct
  REGION 1..11: note = Synthetic
DPQSFSSPFDY

SEQ ID NO: 1838 - protein, 11 aa, synthetic construct
  REGION 1..11: note = Synthetic
DPQSLSSPFDY

SEQ ID NO: 1839 - protein, 11 aa, synthetic construct
  REGION 1..11: note = Synthetic
DDQSISSPLDY

SEQ ID NO: 1840 - protein, 11 aa, synthetic construct
  REGION 1..11: note = Synthetic
DIQSVSGEFDY

SEQ ID NO: 1841 - protein, 11 aa, synthetic construct
  REGION 1..11: note = Synthetic
DPQTLTSPFDY

SEQ ID NO: 1842 - skipped

SEQ ID NO: 1843 - protein, 9 aa, synthetic construct
  REGION 1..9: note = Synthetic
GLYDGTGNI

SEQ ID NO: 1844-1848 - skipped (5 entries)

SEQ ID NO: 1849 - protein, 4 aa, synthetic construct
  REGION 1..4: note = Synthetic
DFYG

SEQ ID NO: 1850-1853 - skipped (4 entries)

SEQ ID NO: 1854 - protein, 12 aa, synthetic construct
  REGION 1..12: note = Synthetic
DFYGWNSGALDI

SEQ ID NO: 1855-1859 - skipped (5 entries)

SEQ ID NO: 1860 - protein, 9 aa, synthetic construct
  REGION 1..9: note = Synthetic
GLYDGTGNI

SEQ ID NO: 1861 - skipped

SEQ ID NO: 1862 - protein, 9 aa, synthetic construct
  REGION 1..9: note = Synthetic
GLYDGTGNI

SEQ ID NO: 1863 - protein, 9 aa, synthetic construct
  REGION 1..9: note = Synthetic
GLYDGTGNI

SEQ ID NO: 1864-1953 - skipped (90 entries)

SEQ ID NO: 1954 - protein, 13 aa, synthetic construct
  REGION 1..13: note = Synthetic
QASQSVYGNNWLS

SEQ ID NO: 1955-1959 - skipped (5 entries)

SEQ ID NO: 1960 - protein, 13 aa, synthetic construct
  REGION 1..13: note = Synthetic
QASQSVYSNKYLS

SEQ ID NO: 1961-1964 - skipped (4 entries)

SEQ ID NO: 1965 - protein, 13 aa, synthetic construct
  REGION 1..13: note = Synthetic
RASQSVYSNKYLS

SEQ ID NO: 1966-1970 - skipped (5 entries)

SEQ ID NO: 1971 - protein, 13 aa, synthetic construct
  REGION 1..13: note = Synthetic
RASQSVYGNNWLS

SEQ ID NO: 1972 - skipped

SEQ ID NO: 1973 - protein, 13 aa, synthetic construct
  REGION 1..13: note = Synthetic
RASQSVYGNNWLS

SEQ ID NO: 1974 - protein, 13 aa, synthetic construct
  REGION 1..13: note = Synthetic
QASQSVYGNNWLS

SEQ ID NO: 1975-2063 - skipped (89 entries)

SEQ ID NO: 2064 - protein, 11 aa, synthetic construct
  REGION 1..11: note = Synthetic
RASQDVNTAVA

SEQ ID NO: 2065 - skipped

SEQ ID NO: 2066 - protein, 13 aa, synthetic construct
  REGION 1..13: note = Synthetic
QASQSVYGNNWLS

SEQ ID NO: 2067-2071 - skipped (5 entries)

SEQ ID NO: 2072 - protein, 13 aa, synthetic construct
  REGION 1..13: note = Synthetic
QASQSVYSNKYLS

SEQ ID NO: 2073-2076 - skipped (4 entries)

SEQ ID NO: 2077 - protein, 13 aa, synthetic construct
  REGION 1..13: note = Synthetic
RASQSVYSNKYLS

SEQ ID NO: 2078-2082 - skipped (5 entries)

SEQ ID NO: 2083 - protein, 13 aa, synthetic construct
  REGION 1..13: note = Synthetic
RASQSVYGNNWLS

SEQ ID NO: 2084 - skipped

SEQ ID NO: 2085 - protein, 13 aa, synthetic construct
  REGION 1..13: note = Synthetic
RASQSVYGNNWLS

SEQ ID NO: 2086 - protein, 13 aa, synthetic construct
  REGION 1..13: note = Synthetic
QASQSVYGNNWLS

SEQ ID NO: 2087-2175 - skipped (89 entries)

SEQ ID NO: 2176 - protein, 11 aa, synthetic construct
  REGION 1..11: note = Synthetic
RASQDVNTAVA

SEQ ID NO: 2177 - skipped

SEQ ID NO: 2178 - protein, 7 aa, synthetic construct
  REGION 1..7: note = Synthetic
GASKLAS

SEQ ID NO: 2179-2183 - skipped (5 entries)

SEQ ID NO: 2184 - protein, 7 aa, synthetic construct
  REGION 1..7: note = Synthetic
KASTLAS

SEQ ID NO: 2185-2188 - skipped (4 entries)

SEQ ID NO: 2189 - protein, 7 aa, synthetic construct
  REGION 1..7: note = Synthetic
KASTLAS

SEQ ID NO: 2190-2194 - skipped (5 entries)

SEQ ID NO: 2195 - protein, 7 aa, synthetic construct
  REGION 1..7: note = Synthetic
GASKLAS

SEQ ID NO: 2196 - skipped

SEQ ID NO: 2197 - protein, 7 aa, synthetic construct
  REGION 1..7: note = Synthetic
GASKLAT

SEQ ID NO: 2198 - protein, 7 aa, synthetic construct
  REGION 1..7: note = Synthetic
GASKLAS

SEQ ID NO: 2199-2287 - skipped (89 entries)

SEQ ID NO: 2288 - protein, 7 aa, synthetic construct
  REGION 1..7: note = Synthetic
SASFLYS

SEQ ID NO: 2289 - skipped

SEQ ID NO: 2290 - protein, 7 aa, synthetic construct
  REGION 1..7: note = Synthetic
GASKLAS

SEQ ID NO: 2291-2295 - skipped (5 entries)

SEQ ID NO: 2296 - protein, 7 aa, synthetic construct
  REGION 1..7: note = Synthetic
KASTLAS

SEQ ID NO: 2297-2300 - skipped (4 entries)

SEQ ID NO: 2301 - protein, 7 aa, synthetic construct
  REGION 1..7: note = Synthetic
KASTLAS

SEQ ID NO: 2302-2306 - skipped (5 entries)

SEQ ID NO: 2307 - protein, 7 aa, synthetic construct
  REGION 1..7: note = Synthetic
GASKLAS

SEQ ID NO: 2308 - skipped

SEQ ID NO: 2309 - protein, 7 aa, synthetic construct
  REGION 1..7: note = Synthetic
GASKLAT

SEQ ID NO: 2310 - protein, 7 aa, synthetic construct
  REGION 1..7: note = Synthetic
GASKLAS

SEQ ID NO: 2311-2399 - skipped (89 entries)

SEQ ID NO: 2400 - protein, 7 aa, synthetic construct
  REGION 1..7: note = Synthetic
SASFLYS

SEQ ID NO: 2401 - skipped

SEQ ID NO: 2402 - protein, 12 aa, synthetic construct
  REGION 1..12: note = Synthetic
QGTYYSGDWYFA

SEQ ID NO: 2403-2407 - skipped (5 entries)

SEQ ID NO: 2408 - protein, 11 aa, synthetic construct
  REGION 1..11: note = Synthetic
AAAYSDDSDTA

SEQ ID NO: 2409-2412 - skipped (4 entries)

SEQ ID NO: 2413 - protein, 11 aa, synthetic construct
  REGION 1..11: note = Synthetic
AAAYSDDSDTA

SEQ ID NO: 2414-2418 - skipped (5 entries)

SEQ ID NO: 2419 - protein, 12 aa, synthetic construct
  REGION 1..12: note = Synthetic
QGTYYSGDWYFA

SEQ ID NO: 2420 - skipped

SEQ ID NO: 2421 - protein, 12 aa, synthetic construct
  REGION 1..12: note = Synthetic
QGTYYSGDWYFA

SEQ ID NO: 2422 - protein, 12 aa, synthetic construct
  REGION 1..12: note = Synthetic
QGTYYSGDWYFA

SEQ ID NO: 2423-2511 - skipped (89 entries)

SEQ ID NO: 2512 - protein, 9 aa, synthetic construct
  REGION 1..9: note = Synthetic
QQHYTTPPT

SEQ ID NO: 2513 - skipped

SEQ ID NO: 2514 - protein, 12 aa, synthetic construct
  REGION 1..12: note = Synthetic
QGTYYSGDWYFA

SEQ ID NO: 2515-2519 - skipped (5 entries)

SEQ ID NO: 2520 - protein, 11 aa, synthetic construct
  REGION 1..11: note = Synthetic
AAAYSDDSDTA

SEQ ID NO: 2521-2524 - skipped (4 entries)

SEQ ID NO: 2525 - protein, 11 aa, synthetic construct
  REGION 1..11: note = Synthetic
AAAYSDDSDTA

SEQ ID NO: 2526-2530 - skipped (5 entries)

SEQ ID NO: 2531 - protein, 12 aa, synthetic construct
  REGION 1..12: note = Synthetic
QGTYYSGDWYFA

SEQ ID NO: 2532 - skipped

SEQ ID NO: 2533 - protein, 12 aa, synthetic construct
  REGION 1..12: note = Synthetic
QGTYYSGDWYFA

SEQ ID NO: 2534 - protein, 12 aa, synthetic construct
  REGION 1..12: note = Synthetic
QGTYYSGDWYFA

SEQ ID NO: 2535-2623 - skipped (89 entries)

SEQ ID NO: 2624 - protein, 9 aa, synthetic construct
  REGION 1..9: note = Synthetic
QQHYTTPPT

SEQ ID NO: 2625-2726 - skipped (102 entries)

SEQ ID NO: 2727 - protein, 123 aa, synthetic construct
  REGION 1..123: note = Synthetic
MAEVQLVESGGGLVQPGGSLRLSCAASGFNISSYWIHWVRQAPGKGLEWVGYIDPYSGSTYYADSVKGRFTISADTSKNTAYLQMNSLRAEDTAVYYCARSDYYYVDEYMDYWGQGTLVTVSS

SEQ ID NO: 2728-2735 - skipped (8 entries)

SEQ ID NO: 2736 - protein, 122 aa, synthetic construct
  REGION 1..122: note = Synthetic
MAEVQLVESGGGLVQPGGSLRLSCAASGFNISDYWIHWVRQAPGKGLEWVGFIDPYNGYTNYADSVKGRFTISADTSKNTAYLQMNSLRAEDTAVYYCARGYGSWPDYLDYWGQGTLVTVSS

SEQ ID NO: 2737-2738 - skipped (2 entries)

SEQ ID NO: 2739 - protein, 122 aa, synthetic construct
  REGION 1..122: note = Synthetic
MAEVQLVESGGGLVQPGGSLRLSCAASGFNISDYNIHWVRQAPGKGLEWVGYIDPSNGYTYYADSVKGRFTISADTSKNTAYLQMNSLRAEDTAVYYCARDDQSVSSPFDYWGQGTLVTVSS

SEQ ID NO: 2740-2750 - skipped (11 entries)

SEQ ID NO: 2751 - protein, 122 aa, synthetic construct
  REGION 1..122: note = Synthetic
MEVQLVESGGGLVQPGGSLRLSCAASGFTISSYWIHWVRQAPGKGLKWVGYIDPYNGATYYADSVKGRFTISADTSKNTAYLQMNSLRAEDTAVYYCARADYYYVDEYFDYWGQGTLVTVSS

SEQ ID NO: 2752 - protein, 121 aa, synthetic construct
  REGION 1..121: note = Synthetic
MEVQLVESGGGLVQPGGSLRLSCAASGFTISGYIIHWVRQAPGKGLEWVGYIDPSNGYTYYADSVKGRFTISADTSKNTAYLQMNSLRAEDTAVYYCARDDQSLSSPFDYWGQGTLVTVSS

SEQ ID NO: 2753 - protein, 122 aa, synthetic construct
  REGION 1..122: note = Synthetic
MEVQLVESGGGLVQPGGSLRLSCAASGFTIPSYWIHWVRQAPGKGLEWVGYIDPANGATYYADSVKGRFTISADTSKNTAYLQMNSLRAEDTAVYYCARADYFYVDEYLDYWGQGTLVTVSS

SEQ ID NO: 2754 - protein, 122 aa, synthetic construct
  REGION 1..122: note = Synthetic
MEVQLVESGGGLVQPGGSLRLSCAASGFTISGYWIHWVRQAPGKGLEWVGYIDPYNGATYYADSVKGRFTISADTSKNAAYLQMNSLRAEDTAVYYCARADYFYIDEYWDYWGQGTLVTVSS

SEQ ID NO: 2755 - protein, 122 aa, synthetic construct
  REGION 1..122: note = Synthetic
MEVQLVESGGGLVQPGGSLRLSCAASGFTIPDYWIHWVRQAPGKGLEWVGFIDPYNGYTNYADPVKSRFTISADTSKNTAYLQMNSLRAEDTAVYYCARADYYYVDEYWDYWGQGTLVTVSS

SEQ ID NO: 2756 - protein, 122 aa, synthetic construct
  REGION 1..122: note = Synthetic
MEVQLVESGGGLVQPGGSLRLSCAASGFTISSYWIHWVRQAPGKGLEWVGYIDPFNGATYYADSVKGRFTISADTSKNTAYLQMNSLRAEDTAVYYCARADYYYVDEYWDYWGQGTLVTVSS

SEQ ID NO: 2757 - protein, 122 aa, synthetic construct
  REGION 1..122: note = Synthetic
MEVQLVESGGGLVQPGGSLRLSCAASGFTISSYWIHWVRQAPGKGLEWVGYIDPYNGATYYADSVKGRFTISADTSKNTAYLQMNSLRAEDTAVYYCARSDYYYVDEFFDYWGQGTLVTVSS

SEQ ID NO: 2758 - protein, 122 aa, synthetic construct
  REGION 1..122: note = Synthetic
MEVQLVESGGGLVQPGGSLRLSCAASGFTLTGYWIHWVRQAPGKGLEWVGYIDPYNGATYYADSVKGRFTISADTSKNTAYLQMNSLRAEDTAVYYCARADYYYVDEYWDYWGQGTLVTVSS

SEQ ID NO: 2759 - protein, 122 aa, synthetic construct
  REGION 1..122: note = Synthetic
MEVQLVESGGGLVQPGGSLRLSCAASGFTIADYWIHWVRQAPGKGLEWVGYIDPYNGATYYADSVKGRFTISADTSKNTAYLQMNSLRAEDTAVYYCARADYYYVDEYWDYWGQGTLVTVSS

SEQ ID NO: 2760 - protein, 122 aa, synthetic construct
  REGION 1..122: note = Synthetic
MEVQLVESGGGLVQPGGALRLSCAASGFTISSYWIHWVRQAPGKGLEWVGYIDPYNGATYYADSVKGRFTISADTSKNTAYLQMNSLRAEDTAVYYCARADYYYVDEYLDYWGQGTLVTVSS

SEQ ID NO: 2761 - protein, 121 aa, synthetic construct
  REGION 1..121: note = Synthetic
MEVQLVESGGGLVQPGGSLRLSCAASGFTITDYVIHWVRQAPGKGLEWVGYIDPSNGYTYYADSVKGRFTISADTSKNTAYLQMNSLRAEDTAVYYCARDDQSFTSPFDYWGQGTLVTVSS

SEQ ID NO: 2762 - protein, 122 aa, synthetic construct
  REGION 1..122: note = Synthetic
MEVQLVESGGGLVQPGGSLRLSCAASGFTISGYWIHWVRQAPGKGLEWVGYIDPYNGATYYADSVKGRFTISADTSKNTAYLQMNSLRAEDTAVYYCARADYYYVDEYLDYWGQGTLVTVSS

SEQ ID NO: 2763 - protein, 122 aa, synthetic construct
  REGION 1..122: note = Synthetic
MEVQLVESGGGSVQPGGSLRLSCAASGFTISDYWIHWVRQAPGKGLEWVGYIDPYNGATYYADSVKGRFTISADTSKNTAYLQMNSLRAEDTAVYYCARADYYYVDEYWDYWGQGTLVTVSS

SEQ ID NO: 2764 - protein, 122 aa, synthetic construct
  REGION 1..122: note = Synthetic
MEVQLVESGGGLVQPGGSLRLSCSASGFTIADYWIHWVRQAPGKGLEWVGYIDPYNGATYYADSVKGRFTISADTSKNTAYLQMNSLRAEDTAVYYCARADYYYVDEYWDYWGQGTLVTVSS

SEQ ID NO: 2765 - protein, 122 aa, synthetic construct
  REGION 1..122: note = Synthetic
MEVQLVESGGGLVQPGGSLRLSCAASGFTISEYWIHWVRQAPGKGLEWVGFIDPYNGYTGYADSVRGRFTISADTSKNTAYLQMNSLRAEDTAVYYCARADYYYVDEYWDYWGQGTLVTVSS

SEQ ID NO: 2766 - protein, 122 aa, synthetic construct
  REGION 1..122: note = Synthetic
MEVQLVESGGGLVQPGGSLRLSCAASGFTISGYWIHWVRQAPGKGLEWVGYIDPYNGATYYADSVKGRFTISADTSKNTAYLQMNSLRAEDTAVYYCARADYYGVDEFLDYWGQGTLVTVSS

SEQ ID NO: 2767 - protein, 122 aa, synthetic construct
  REGION 1..122: note = Synthetic
MEVQLVESGGGLVQPGGSLRLSCAASGFTIPSYWIHWVRQAPGKGLEWVGYIDPYNGATYYADSVKGRFTISADTSKNTAYLQMNSLRAEDTAVYYCARADYHGVDEYWDYWGQGTLVTVSS

SEQ ID NO: 2768 - protein, 122 aa, synthetic construct
  REGION 1..122: note = Synthetic
MEVQLVESGGGLVQPGGSLRLSCAASGFTIADYWIHWVRQAPGKGLEWVGFIDPYNGYTGYADSVKGRFTISADNSKNTAYLQMNSLRAEDTAVYYCARADYHGVDEYWDYWGQGTLVTVSS

SEQ ID NO: 2769 - protein, 122 aa, synthetic construct
  REGION 1..122: note = Synthetic
MEVQLVESGGGLVQPGGSLRLSCAASGFTIPSYWIHWVRQAPGKGLEWVGYIDPYNGYTYYADSVKGRFTISADTSKNTAYLQMNSLRAEDTAVYYCARADYYYVDEYFDYWGQGTLVTVSS

SEQ ID NO: 2770 - protein, 122 aa, synthetic construct
  REGION 1..122: note = Synthetic
MEVQLVESGGGLVQPGGSLRLSCAASGFTISQYWIHWVRQAPGKGLEWVGFIDPYNGYTNYADSVKGRFTISADTSENTAYLQMNSLRAEDTAVYYCARADYYYVDEYWDYWGQGTLVTVSS

SEQ ID NO: 2771 - protein, 122 aa, synthetic construct
  REGION 1..122: note = Synthetic
MEVQLVESGGGLVQPGGSLRLSCAASGFTIPAYWIHWVRQAPGKGLEWVGYIDPYNGSTYYADSVKGRFTISADTSKNTAYLQMNSLRAEDTAVYYCARADYYYVDEYWDYWGQGTLVTVSS

SEQ ID NO: 2772 - protein, 122 aa, synthetic construct
  REGION 1..122: note = Synthetic
MEVQLVESGGGLVQPGGSLRLSCAASGFTIPAYWIHWVRQAPGKGLEWVGYIDPSNGYTYYADSVKGRFTISADASKNTAYLQMNSLRAEDTAAYYCARADYHGVDEYWDYWGQGTLVTVSS

SEQ ID NO: 2773 - protein, 122 aa, synthetic construct
  REGION 1..122: note = Synthetic
MEVQLVESGGGLVQPGGSLRLSCAASGFTIGSYWIHWVRQAPGKGLEWVGYIDPSNGYTYYADSVKGRFTISADTSKNTAYLQMNSLRAEDTAVYYCARADYYYVDEYWDYWGQGTLVTVSS

SEQ ID NO: 2774 - protein, 122 aa, synthetic construct
  REGION 1..122: note = Synthetic
MEVQLVESGGGLVQPGGSLRLSCAASGFTISSYWIHWVRQAPGKGLEWVGYIDPYNGATYYADSVKGRFTISADTSKNTAYLQMNSLRAEDTAVYYCARADYYYVDEYWDYWGQGTLVTVSS

SEQ ID NO: 2775 - protein, 122 aa, synthetic construct
  REGION 1..122: note = Synthetic
MEVQLVESGGGLVQPGGSLRLSCAASGFTISGYWIHWVRQAPGKGLEWVGYIDPYNGATYYADSVKGRFTISADTSKNTAYLQMNSLRAEDTAVYYCARADYYYVDEYWDYWGQGTLVTVSS

SEQ ID NO: 2776 - protein, 122 aa, synthetic construct
  REGION 1..122: note = Synthetic
MEVQLVESGGGLVQPGGSLRLSCAASGFTIVSYWIHWVRQAPGKGLEWVGYIDPYNGATYYADSVKGRFTISADTSKNTAYLQMNSLRAEDTAVYYCARADYYYVDEYWDYWGQGTLVTVSS

SEQ ID NO: 2777 - protein, 122 aa, synthetic construct
  REGION 1..122: note = Synthetic
MEVQLVESGGGLVQPGGSLRLSCAASGFTISGYWIHWVRQAPGKGLEWVGYIDPYNGATYYADSVKGRFTISADTPKNTAYLQMNSLRAEDTAVYYCARADYHGVDEYWDYWGQGTLVTVSS

SEQ ID NO: 2778 - protein, 122 aa, synthetic construct
  REGION 1..122: note = Synthetic
MEVQLVESGGGLVQPGGSLRLSCAASGFTISGYWIHWVRQAPGKGLEWVGYIDPYNGATFYADSVKGRFTISADTSKNTAYLQMNSLRAEDTAVYYCARADYYYVDEYWDYWGQGTLVTVSS

SEQ ID NO: 2779 - protein, 122 aa, synthetic construct
  REGION 1..122: note = Synthetic
MEVQLVESGGGLVQPGGSLRLSCAASGFTISGYWIHWVRQAPGKGLEWVGYIDPYNGATYYADSVKGRFTISADTSKNTAYLQMNSLRAEDTAVYYCARADYYYVDEYFDYWGQGTLVTVSS

SEQ ID NO: 2780 - protein, 122 aa, synthetic construct
  REGION 1..122: note = Synthetic
MEVQLVESGGGLVQPGGSLRLSCAASGFTIPAYWIHWVRQAPGKGLEWVGYIDPYNGSTYHADSVKGRFTISADTSKNTAYLQMNSLRAEDTAVYYCARADYYYVDEYWDYWGQGTLVTVSS

SEQ ID NO: 2781 - protein, 121 aa, synthetic construct
  REGION 1..121: note = Synthetic
MEVQLVESGGGLVQPGGSLRLSCAASGFTISDYIIHWVRQAPGKGLEWVGYIDPSNGYTYHADSVKGRFTISADTSKNTAYLQMNSLRAEDTAVYYCARDDQSFSSPFDYWGQGTLVTVSS

SEQ ID NO: 2782 - protein, 121 aa, synthetic construct
  REGION 1..121: note = Synthetic
MEVQLVESGGGLVQPGGSLRLSCAASGFTISDYWIHWVRQAPGKGLEWVGYIDPYNGYTYYADSVNGRFTISADTSKNTAYLQMNSLRAEDTAVYYCARDYGSYLVSLDYWGQGTLVTVSS

SEQ ID NO: 2783 - protein, 121 aa, synthetic construct
  REGION 1..121: note = Synthetic
MEVQLVESGGGLVQPGGSLRLSCAASGFTISQYWIHWVRQAPGKGLEWVGYIDPYNGYTYYADSVKGRFTISADTSKNTAYLQMNSLRAEDTAVYYCARDYGSWRIRLDYWGQGTLVTVSS

SEQ ID NO: 2784 - protein, 121 aa, synthetic construct
  REGION 1..121: note = Synthetic
MEVQLVESGGGLVQPGGSLRLSCAASGFTIHEYWIHWVRQAPGKGLEWVGYIDPANGFTYYADSVKGRFTISADTSKNTAYLQMNSLRAEDTAVYYCARDYGSLQVFLDYWGQGTLVTVSS

SEQ ID NO: 2785 - protein, 121 aa, synthetic construct
  REGION 1..121: note = Synthetic
MEVQLVESGGGLVQPGGSLRLSCAASGFTISDYWIHWVRQAPGKGLEWVGYIDPYNGYTYYADSVKGRFTISADTSKNTAYLQMNSLRAEDTAVYYCARDYGSLQVFIDHWGQGTLVTVSS

SEQ ID NO: 2786 - protein, 121 aa, synthetic construct
  REGION 1..121: note = Synthetic
MEVQLVESGGGLVQPGGSLRLSCAASGFTISQYWIHWVRQAPGKGLEWVGYIDPYNGYTYYADSVKGRFTISADTSKNTAYLQMNSLRAEDTAVYYCARDYGSLQVQLDYWGQGTLVTVSS

SEQ ID NO: 2787 - protein, 121 aa, synthetic construct
  REGION 1..121: note = Synthetic
MEVQLVESGGGLVQPGGSLRLSCAASGFTISDYWIHWVRQAPGKGLEWVGFIDPYNGYTYHADSVEGRFTISADTSKNTAYLQMNSLRAEDTAVYYCARDYGSLQVQLDYWGQGTLVTVSS

SEQ ID NO: 2788 - protein, 121 aa, synthetic construct
  REGION 1..121: note = Synthetic
MEVQLVESGGGLVQPGGSLRLSCAASGFAIANYWIHWVRQAPGKGLEWVGYIDPYNGYTYYADSVKGRFTISADTSKNTAYLQMNSLRAEDTAVYYCARDYGSLEVSLDYWGQGTLVTVSS

SEQ ID NO: 2789 - protein, 121 aa, synthetic construct
  REGION 1..121: note = Synthetic
MEVQLVESGGGLVQPGGSLRLSCAASGFTISDYWIHWVRQAPGKGLEWVGYIDPQNGWTYYADSVKGRFTISADTSKNTAYLQMNSLRAEDTAVYYCARDYGSLTINLDYWGQGTLVTVSS

SEQ ID NO: 2790 - protein, 121 aa, synthetic construct
  REGION 1..121: note = Synthetic
MEVQLVESGGGLVQPGGSLRLSCAASGFTISDYWIHWVRQAPGKGLEWVGYIDPSNGYTYYADSVKGRFTISADTSRNTAYLQMNSLRAEDTAVYYCARDYGSLEVSLDYWGQGTLVTVSS

SEQ ID NO: 2791 - protein, 121 aa, synthetic construct
  REGION 1..121: note = Synthetic
MEVQLVESGGGLVQPGGSLRLSCAASGFTISDYWIHWVRQAPGKGLEWVGYIDPYNGYTYYADSVKGRFTISADTSKNTAYLQMNSLRAEDTAVYYCARDYGSLEVSLDYWGQGTLVTVSS

SEQ ID NO: 2792 - protein, 121 aa, synthetic construct
  REGION 1..121: note = Synthetic
MEVQLVESGGGLVQPGGSLRLSCAASGFTISGYWIHWVRQAPGKGLEWVGYIDPANGFTYYADSVKGRFTISADTSKNTAYLQMNSLRAEDTAVYYCARDYGSIQVFLDYWGQGTLVTVSS

SEQ ID NO: 2793 - protein, 121 aa, synthetic construct
  REGION 1..121: note = Synthetic
MEVQLVESGGGLVQPGGSLRLSCAASGFTISDYWIHWVRQAPGKGLEWVGFIDPKNGYTYYADSVKGRFTISAGTSKNTAYLQMNSLRAEDTAVYYCARDYGSLQVFIDYWGQGTLVTVSS

SEQ ID NO: 2794 - protein, 121 aa, synthetic construct
  REGION 1..121: note = Synthetic
MEVQLVESGGGLVQPGGSLRLSCAASGFTISDYWIHWVRQAPGKGLEWVGYIDPYNGYTYYADSVKGRFTISADTSKNTAYLQMNPLRAEDTAVYYCARDYGSLQVQLDYWGQGTLVTVSS

SEQ ID NO: 2795 - protein, 121 aa, synthetic construct
  REGION 1..121: note = Synthetic
MEVQLVESGGGLVQPGGSLRLSCAASGFTISDYWIHWVRQAPGKGLEWVGYIDPSNGYTYYADSVKGRFTISADTSKNTAYLQMNSLRAEDTAVYYCARDYGSLQVFIDYWGQGTLVTVSS

SEQ ID NO: 2796 - protein, 121 aa, synthetic construct
  REGION 1..121: note = Synthetic
MEVQLVESGGGLVQPGGSLRLSCAASGFTISDYVIHWVRQAPGKGLEWVGYIDPSNGYTYYADPVKGRFTISADTSKNTAYLQMNSLRAEDTAVYYCARDPQSFYIAFDYWGQGTLVTVSS

SEQ ID NO: 2797 - protein, 121 aa, synthetic construct
  REGION 1..121: note = Synthetic
MEVQLVESGGGLVQPGGSLRLSCAASGFTISDYWIHWVRQAPGKGLEWVGYIDPQNGWTYYADSVKGRFTISADTSKNTAYLQMNSLRAEDTAVYYCARDYGSLEVSLDYWGQGTLVTVSS

SEQ ID NO: 2798 - protein, 122 aa, synthetic construct
  REGION 1..122: note = Synthetic
MEVQLVESGGGLVQPGGSLRLSCAASGFTIPDYWIHWVRQAPGKGLEWVGYIDPYSGSTFYADSVKGRFTISADTSKNTAYLQMNSLRAEDTAVYYCARADYYYVDEYLDYWGQGTLVTVSS

SEQ ID NO: 2799 - protein, 121 aa, synthetic construct
  REGION 1..121: note = Synthetic
MEVQLVESGGGLVQPGGSLRLSCAASGFTISGYWIHWVRQAPGKGLEWVGYIDPANGFTYYADSVKGRFTISADTSKNTAYLQMNSLRAEDTAVYYCARDYGSVRVFLDYWGQGTLVTVSS

SEQ ID NO: 2800 - protein, 121 aa, synthetic construct
  REGION 1..121: note = Synthetic
MEVQLVESGGGLVQPGGSLRLSCAASGFTISGYIIHWVRQAPGKGLEWVGYIDPSNGYTYHADSVKGRFTISADTSKNTAYLQMNSLRAEDTAVYYCARDDQSVSSPFDYWGQGTLVTVSS

SEQ ID NO: 2801 - protein, 121 aa, synthetic construct
  REGION 1..121: note = Synthetic
MEVQLVESGGGLVQPGGSLRLSCAASGFTISQYWIHWVRQAPGKGLEWVGYIDPYNGYTYYADSVKGRFTISADTSKNTAYLQMNSLRAEDTAVYYCARDYGSLRVSLDYWGQGTLVTVSS

SEQ ID NO: 2802 - protein, 121 aa, synthetic construct
  REGION 1..121: note = Synthetic
MEVQLVESGGGLVQPGGSLRLSCAASGFTITDYWIHWVRQAPGKGLEWVGYIDPYNGYTYYADSVKGRFTISADTSKNTAYLQMNSLRAEDTAVYYCARDYGSLQVFIDYWGQGTLVTVSS

SEQ ID NO: 2803 - protein, 121 aa, synthetic construct
  REGION 1..121: note = Synthetic
MEVQLVESGGGLVQPGGSLRLSCAASGFTISQYWIHWVRQAPGKGLEWVGYIDPYNGYTYYADSVKGRFTISADTSKNTAHLQMNSLRAEDTAVYYCARDYGSFRVQLDYWGQGTLVTVSS

SEQ ID NO: 2804 - protein, 121 aa, synthetic construct
  REGION 1..121: note = Synthetic
MEVQLVESGGGLVQPGGSLRLSCAASGFTISDYWIHWVRQAPGKGLEWVGYIDPANGYTYYADSVKGRFTISADTSKNTAYLQMNSLRAEDTAVYYCARDYGSLHVQLDYWGQGTLVTVSS

SEQ ID NO: 2805 - protein, 121 aa, synthetic construct
  REGION 1..121: note = Synthetic
MEVQLVESGGGLVQPGGSLRLSCAASGFTISYYIIHWVRQAPGKGLEWVGYIDPSNGYTYYADSVKGRFTISADTSKNTAYLQMNSLRAEDTAVYYCARDVQSVSGEFDYWGQGTLVTVSS

SEQ ID NO: 2806 - protein, 121 aa, synthetic construct
  REGION 1..121: note = Synthetic
MEVQLVESGGGLVQPGGSLRLSCAASGFTISDYWIHWVRQAPGKGLEWVGYIDPYNGYTYYADSVRGRFTISADTSKNTAYLQMNSLRAEDTAVYYCARDYGSLQVFIDYWGQGTLVTVSS

SEQ ID NO: 2807 - protein, 121 aa, synthetic construct
  REGION 1..121: note = Synthetic
MEVQLVESGGGLVQPGGSLRLACAASGFTIGVYIIHWVRQAPGKGLEWVGYIDPSNGYTYYADSVKGRFTISADTSKNTAYLQMNSLRAEDTAVYYCARDDQSVSSPFDYWGQGTLVTVSS

SEQ ID NO: 2808 - protein, 121 aa, synthetic construct
  REGION 1..121: note = Synthetic
MEVQLVESGGGLVQPGGSLRLSCAASGFTISDYWIHWVRQAPGKGLEWVGYIDPYNGYTYYADSVKGRFTISAGTSKNTAYLQMNSLRAEDTAVYYCARDYGSLQVFIDYWGQGTLVTVSS

SEQ ID NO: 2809 - protein, 121 aa, synthetic construct
  REGION 1..121: note = Synthetic
MEVQLVESGGGLVQPGGSLRLSCAASGFTISDYWIHWVRQAPGKGLEWVGYIDPYNGFTYYADSVKGRFTISADTSKNTAYLQMNSLRAEDTAVYYCARDYGSLQVFIDYWGQGTLVTVSS

SEQ ID NO: 2810 - protein, 121 aa, synthetic construct
  REGION 1..121: note = Synthetic
MEVQLVESGGGLVQPGGSLRLSCAASGFTINDYVIHWVRQAPGKGLEWVGYIDPSNGYTYYADSVKGRFTISADTSKNTAYLQMNSLRAEDTAVYYCARDDQSLSSPFDYWGQGTLVTVSS

SEQ ID NO: 2811 - protein, 121 aa, synthetic construct
  REGION 1..121: note = Synthetic
MEVQLVESGGGLVQPGGSLRLSCAASGFTISIYTIHWVRQAPGKGLEWVGYIDPSNGYTYYADSVKGRLTISADTSKNTAYLQMNSLRAEDTAVYYCARDPQSISSPFDYWGQGTLVTVSS

SEQ ID NO: 2812 - protein, 121 aa, synthetic construct
  REGION 1..121: note = Synthetic
MEVQLVESGGGLVQPGGSLRLSCAASGFTISDYTIHWVRQAPGKGLEWVGYIDPSNGYTYHADSVKGRFTISADTSKNTAYLQMNSLRAEDTAVYYCARDPQSVSSPFDYWGQGTLVTVSS

SEQ ID NO: 2813 - protein, 121 aa, synthetic construct
  REGION 1..121: note = Synthetic
MEVQLVESGGGLVQPGGSLRLSCAASGFTIAYYIIHWVRQAPGKGLEWVGYIDPSNGYTYYADSVKGRFTISADTSKNTAYLQMNSLRAEDTAVYYCARDDQSLSSPFDYWGQGTLVTVSS

SEQ ID NO: 2814 - protein, 121 aa, synthetic construct
  REGION 1..121: note = Synthetic
MEVQLVESGGGLVQPGGSLRLSCAASGFTIHDYVIHWVRQAPGKGLEWVGYIDPSNGYTYYADSVKGRFTISADTSKNTAYLQMNSLRAEDTAVYYCARDDQSISSPLDYWGQGTLVTVSS

SEQ ID NO: 2815 - protein, 121 aa, synthetic construct
  REGION 1..121: note = Synthetic
MEVQLVESGGGLVQPGGSLRLSCAASGFTISDYVIHWVRQAPGKGLEWVGYIDPSNGYTYYADSVKGRFTISADTSKNTAYLQMNSLRAEDTAVYYCARDPQSLFYPFDYWGQGTLVTVSS

SEQ ID NO: 2816 - protein, 121 aa, synthetic construct
  REGION 1..121: note = Synthetic
MEVQLVESGGGLVQPGGSLRLSCAASGFTISGYIIHWVRQAPGKGLEWVGYIDPSNGYTFYADSVKGRFTISADTSKNTAYLRMNSLRAEDTAVYYCARDDQSLSSPFDYWGQGTLVTVSS

SEQ ID NO: 2817 - protein, 121 aa, synthetic construct
  REGION 1..121: note = Synthetic
MEVQLVESGGGLVQPGGSLRLSCAASGFTIFDYTIHWVRQAPGKGLEWVGYIDPSNGYTFYADSVKGRFTISADTSKNTAYLQVNSLRAEDTAVYYCARDIQSVSGEFDYWGQGTLVTVSS

SEQ ID NO: 2818 - protein, 121 aa, synthetic construct
  REGION 1..121: note = Synthetic
MEVQLVESGGDLVRPGGSLRLSCAASGFTISYYIIHWVRQAPGKGLEWVGYIDPSNGYTYYADSVKGRFTISADTSKNTAYLQMNSLRAEDTAVYYCARDDQSVSSPFEYWGQGTLVTVSS

SEQ ID NO: 2819 - protein, 121 aa, synthetic construct
  REGION 1..121: note = Synthetic
MEVQLVESGGGLVQPGGSLRLSCAASGFTINNYIIHWVRQAPGKGLEWVGYIDPSNGYTYYADSVKGRFTISADTSKNTAYLQMNSLRAEDTAVYYCARDPQSLSSPFDYWGQGTLVTVSS

SEQ ID NO: 2820 - protein, 121 aa, synthetic construct
  REGION 1..121: note = Synthetic
MEVQLVESGGGLVQPGGSLRLSCAASGFTTRDYVIHWVRQAPGKGLEWVGYIDPSNGYTYYADSVKGRFTISADTSKNTAYLQMNSLRAEDTAVYYCARDPQSLSSPFDYWGQGTLVTVSS

SEQ ID NO: 2821 - protein, 121 aa, synthetic construct
  REGION 1..121: note = Synthetic
MEVQLVESGGGLVQPGGLLRLSCAASGFTIAYYVIHWVRQAPGKGLEWVGYIDPSNGYTYYADSVKGRFTISADTSKNTAYLQMNSLRAEDTAVYYCARDDQSLSSPFDYWGQGTLVTVSS

SEQ ID NO: 2822 - protein, 121 aa, synthetic construct
  REGION 1..121: note = Synthetic
MEVQLVESGGGLVQPGGSLRLSCAASGFTISDYIIHWVRQAPGKGLEWVGYIDPSNGYTYYADSVKGRFTISADTSKNTAYLQMNSLRAEDTAVYYCARDDQSLSSPFDYWGQGTLVTVSS

SEQ ID NO: 2823 - protein, 121 aa, synthetic construct
  REGION 1..121: note = Synthetic
MEVQLVESGGGLVQPGGSLRLSCAASGFTISDYVIHWVRQAPGKGLEWVGYIDPSNGYTYYADSVKGRFTISADTSKNTAYLQMNSLRAEDTAVYYCARDPQSLTSPFDYWGQGTLVTVSS

SEQ ID NO: 2824 - protein, 121 aa, synthetic construct
  REGION 1..121: note = Synthetic
MEVQLVESGGGLVQLGGSLRLSCAASGFTIGGYIIHWVRQAPGKGLEWVGYIDPSNGYTYYADSVKGRFTISADTSKNTAYLQMNSLRAEDTAVYYCARDIQSVSGEFDYWGQGTLVTVSS

SEQ ID NO: 2825 - protein, 121 aa, synthetic construct
  REGION 1..121: note = Synthetic
MEVQLVESGGGLVQPGGSLRLSCAASGFTISDYIIHWVRQAPGKGLEWVGYIDPSNGYTYYADSVKGRFTISADTSKNTAYLQMNSLRAEDTAVYYCARDPQSLTSPFDYWGQGTLVTVSS

SEQ ID NO: 2826 - protein, 121 aa, synthetic construct
  REGION 1..121: note = Synthetic
MEVQLVESGGGLVQPGGSLRLSCAASGFTIPDYIIHWVRQAPGKGLEWVGYIDPSNGYTYYADSVKGRFTISADTSKNTAYLQMNSLRAEDTAVYYCARDIQSVSGEFDYWGQGTLVTVSS

SEQ ID NO: 2827 - protein, 121 aa, synthetic construct
  REGION 1..121: note = Synthetic
MEVQLVESGGGLVQPGGSSRLSCAASGFTIYDYIIHWVRQAPGKGLEWVGYIDPSNGYTDYADSVKGRFTISADTSKNTAYLQMNSLRAEDTAVYYCARDIQSVSGEFDYWGQGTLVTVSS

SEQ ID NO: 2828 - protein, 121 aa, synthetic construct
  REGION 1..121: note = Synthetic
MEVQLVESGGGLVQPGGSLRLSCAASGFTIFDYIIHWVRQAPGKGLEWVGYIDPSNGYTYYADSVKGRFTISADTSKNTAYLQMNSLRAEDTAVYYCARDPQSVGSEFDYWGQGTLVTVSS

SEQ ID NO: 2829 - protein, 121 aa, synthetic construct
  REGION 1..121: note = Synthetic
MEVQLVESGGGLVQPGGSLRLSCAASGFTIFDYIIHWVRQAPGKGLEWVGYIDPSNGYTYYADSVKGRFTISADTSKNTAYLQMNSLRAEDTAVYYCARDIQSVSGEFDYWGQGTLVTVSS

SEQ ID NO: 2830 - protein, 121 aa, synthetic construct
  REGION 1..121: note = Synthetic
MEVQLVESGGGLVQPGGSLRLSCAASGFTIYDYIIHWVRQAPGKGLEWVGYIDPSNGYTYHADSVKGRFTISADTSKNTAYLQMNSLRAEDTAVYYCARDPQSLSSPFDYWGQGTLVTVSS

SEQ ID NO: 2831 - protein, 121 aa, synthetic construct
  REGION 1..121: note = Synthetic
MEVQLVESGGGLVQPGGSLRLSCAASGFTISHYIIHWVRQAPGKGLEWVGYIDPSNGYTYYADSVKGRFTISADTSKNTAYLQMNSLRAEDTAVYYCARDIQSVSGEFDYWGQGTLVTVSS

SEQ ID NO: 2832 - protein, 121 aa, synthetic construct
  REGION 1..121: note = Synthetic
MEVQLVESGGGLVQPGGSLRLSCAASGFTISDYTIHWVRQAPGKGLEWVGYIDPSNGYTYYADSVKGRFTISADTSKNTAYLQMNSLRAEDTAVYYCARDIQSVSGEFDYWGQGTLVTVSS

SEQ ID NO: 2833 - protein, 121 aa, synthetic construct
  REGION 1..121: note = Synthetic
MEVQLVESGGGLVQPGGSLRLSCAASGFTIAAYIIHWVRQAPGKGLEWVGYIDPSNGYTYYADSVKGRFTISADTSKNTAYLQMNSLRAEDTAVYYCARDDQSFSSPFDYWGQGTLVTVSS

SEQ ID NO: 2834 - protein, 121 aa, synthetic construct
  REGION 1..121: note = Synthetic
MEVQLVESGGGLVQPGGSLRLSCAASGFNIAYYTIHWVRQAPGKGLEWVGYIDPSNGYTYYADSVKGRFTISADTSKNTAYLQMNSLRAEDTAVYYCARDPQSFSSPFDYWGQGTLVTVSS

SEQ ID NO: 2835 - protein, 121 aa, synthetic construct
  REGION 1..121: note = Synthetic
MEVQLVESGGGLVQPGGSLRLSCAASGFTISDYVIHWVRQAPGKGLEWVGYIDPSNGYTFYADSVKGRFTISADTPKNTAYLQMNSLRAKDTAVYYCARDPQSLSSPFDYWGQGTLVTVSS

SEQ ID NO: 2836 - protein, 121 aa, synthetic construct
  REGION 1..121: note = Synthetic
MEVQLVESGGGLVQPGGSLRLSCAASGFTISDYVIHWVRQAPGKGLEWVGYIDPSNGYTYYADSVKGRFTISADTSKNTAYLQMNSLRAEDTAVYYCARDDQSISSPLDYWGQGTLVTVSS

SEQ ID NO: 2837 - protein, 121 aa, synthetic construct
  REGION 1..121: note = Synthetic
MEVQLVESGGGLVQPGGSLRLSCAASGFTISDYVIHWVRQAPGKGLEWVGYIDPSNGFTYYADSVKGRFTISADTSKNTAYLQTNSLRAEDTAVYYCARDIQSVSGEFDYWGQGTLVTVSS

SEQ ID NO: 2838 - protein, 121 aa, synthetic construct
  REGION 1..121: note = Synthetic
MEVQLVESGGGLVQPGGSLRLSCAASGFTISSYIIHWVRQAPGKGPEWVGYIDPSNGYTYYADSVKGRFTISADTSKNTAYLQMNSLRAEDTAVYYCARDPQTLTSPFDYWGQGTLVTVSS

SEQ ID NO: 2839 - skipped

SEQ ID NO: 2840 - protein, 116 aa, synthetic construct
  REGION 1..116: note = Synthetic
QSVEESEGGLFKPADTLTLTCTASGFSISSYDMSWVRQAPGNGLEWIGIIGSNGRTYYPNWAKSRSTITRNTNENTVTLKMTSLTAADTATYFCARGLYDGTGNIWGPGTLVTVSS

SEQ ID NO: 2841-2845 - skipped (5 entries)

SEQ ID NO: 2846 - protein, 119 aa, synthetic construct
  REGION 1..119: note = Synthetic
QSVEESEGGLFKPTDTLTLTCTVSGFSLSYYGVSWVRQAPGNGLEWIGTIDSSGRTYYASWANGRFTISNDNAPNTVSLQVNSLAAADTATYFCARDFYGWNSGALDIWGPGTLVTVSS

SEQ ID NO: 2847-2850 - skipped (4 entries)

SEQ ID NO: 2851 - protein, 121 aa, synthetic construct
  REGION 1..121: note = Synthetic
MQVQVQESGPGLVKPPGTLSLTCAVSGFSLSYYGVSWVRQPPGKGLEWIGTIDSSGRTYYNPSLKSRVTISNDNAPNTVSLKLSSVTAADTAVYYCARDFYGWNSGALDIWGQGTLVTVSS

SEQ ID NO: 2852-2856 - skipped (5 entries)

SEQ ID NO: 2857 - protein, 118 aa, synthetic construct
  REGION 1..118: note = Synthetic
MEVQLVESGGGLVQPGGSLRLSCAASGFSISSYDMSWVRQAPGKGLEWIGIIGSNGRTYYPDSVKGRFTISRDNSKNTVTLQMNSLRAEDTAVYYCARGLYDGTGNIWGQGTLVTVSS

SEQ ID NO: 2858 - skipped

SEQ ID NO: 2859 - protein, 118 aa, synthetic construct; identical to SEQ ID NO: 2857
  REGION 1..118: note = Synthetic

SEQ ID NO: 2860 - protein, 118 aa, synthetic construct
  REGION 1..118: note = Synthetic
MQVQVVESGGGLVKPGGSLRLSCAASGFSISSYDMSWIRQAPGKGLEWIGIIGSNGRTYYADSVKGRFTISRNTNENTVYLQMNSLRAEDTAVYYCARGLYDGTGNIWGQGTLVTVSS

SEQ ID NO: 2861-2950 - skipped (90 entries)

SEQ ID NO: 2951 - protein, 112 aa, synthetic construct
  REGION 1..112: note = Synthetic
ELVMTQTPSPVSAAVGSTVTISCQASQSVYGNNWLSWFQQKPGQPPKLLIYGASKLASGVSSRFKGSGSGTQFTLTISGVQCDDAATYYCQGTYYSGDWYFAFGGGTELEIL

SEQ ID NO: 2952-2956 - skipped (5 entries)

SEQ ID NO: 2957 - protein, 111 aa, synthetic construct
  REGION 1..111: note = Synthetic
ELDLTQTASPVSAAVGGTVTINCQASQSVYSNKYLSWYQQKPGQPPKLLIYKASTLASGVPSRFSGSGFGTEFTLTISDVQCDDAATYYCAAAYSDDSDTAFGGGTKVVVE

SEQ ID NO: 2958-2961 - skipped (4 entries)

SEQ ID NO: 2962 - protein, 112 aa, synthetic construct
  REGION 1..112: note = Synthetic
MDLQLTQSPSTLSASVGDRVTITCRASQSVYSNKYLSWYQQKPGKAPKLLIYKASTLASGVPSRFSGSGSGTEFTLTISSLQPDDFATYYCAAAYSDDSDTAFGQGTKVEIK

SEQ ID NO: 2963-2967 - skipped (5 entries)

SEQ ID NO: 2968 - protein, 113 aa, synthetic construct
  REGION 1..113: note = Synthetic
MDLQMTQSPSSLSASVGDRVTITCRASQSVYGNNWLSWYQQKPGKAPKLLIYGASKLASGVPSRFSGSGSGTQFTLTISSLQPEDFATYYCQGTYYSGDWYFAFGQGTKVEIK

SEQ ID NO: 2969 - skipped

SEQ ID NO: 2970 - protein, 113 aa, synthetic construct
  REGION 1..113: note = Synthetic
MELVMTQSPPTLSLSPGERVTLSCRASQSVYGNNWLSWYQQKPGQAPRLLIYGASKLATSIPARFSGSGSGTQFTLTISSLQPEDFAVYYCQGTYYSGDWYFAFGQGTKVEIK

SEQ ID NO: 2971 - protein, 113 aa, synthetic construct
  REGION 1..113: note = Synthetic
MDIQMTQSPSSLSASVGDRVTITCQASQSVYGNNWLSWYQQKPGKAPKLLIYGASKLASGVPSRFSGSGSGTDFTLTISSLQPEDFATYYCQGTYYSGDWYFAFGGGTKVEIK

SEQ ID NO: 2972-3060 - skipped (89 entries)

SEQ ID NO: 3061 - protein, 107 aa, synthetic construct [sequence omitted]

SEQ ID NO: 3062 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3063 - protein, 107 aa, Homo sapiens [sequence omitted]

SEQ ID NO: 3064 - protein, 323 aa, Mus musculus
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 3065 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3066 - protein, 108 aa, synthetic construct
  REGION 1..108: note = Synthetic
HMTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3067 - protein, 106 aa, synthetic construct
  REGION 1..106: note = Synthetic
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3068 - protein, 241 aa, synthetic construct
  REGION 1..241: note = Synthetic
DKTHTCPPCSAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGDYKDDDDKGSG

SEQ ID NO: 3069 - protein, 20 aa, synthetic construct
  REGION 1..20: note = Synthetic
GSGDYKDDDDKGSGHHHHHH

SEQ ID NO: 3070 - protein, 15 aa, synthetic construct
  REGION 1..15: note = Synthetic
GGGGSGGGGSGGGGS

SEQ ID NO: 3071 - protein, 11 aa, synthetic construct
  REGION 1..11: note = Synthetic
AAGSDQEPKSS

SEQ ID NO: 3072 - skipped

SEQ ID NO: 3073 - protein, 249 aa, synthetic construct
  REGION 1..249: note = Synthetic
MSKNKQSVEESEGGLFKPADTLTLTCTASGFSISSYDMSWVRQAPGNGLEWIGIIGSNGRTYYPNWAKSRSTITRNTNENTVTLKMTSLTAADTATYFCARGLYDGTGNIWGPGTLVTVSSGGGGSGGGGSGGGGSELVMTQTPSPVSAAVGSTVTISCQASQSVYGNNWLSWFQQKPGQPPKLLIYGASKLASGVSSRFKGSGSGTQFTLTISGVQCDDAATYYCQGTYYSGDWYFAFGGGTELEILA

SEQ ID NO: 3074-3078 - skipped (5 entries)

SEQ ID NO: 3079 - protein, 251 aa, synthetic construct
  REGION 1..251: note = Synthetic
MSKNKQSVEESEGGLFKPTDTLTLTCTVSGFSLSYYGVSWVRQAPGNGLEWIGTIDSSGRTYYASWANGRFTISNDNAPNTVSLQVNSLAAADTATYFCARDFYGWNSGALDIWGPGTLVTVSSGGGGSGGGGSGGGGSELDLTQTASPVSAAVGGTVTINCQASQSVYSNKYLSWYQQKPGQPPKLLIYKASTLASGVPSRFSGSGFGTEFTLTISDVQCDDAATYYCAAAYSDDSDTAFGGGTKVVVEA

SEQ ID NO: 3080-3159 - skipped (80 entries)

SEQ ID NO: 3160 - protein, 294 aa, Mus musculus
MAILVRPRLLAALAPTFLGCLLLQVIAGAGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGETLIIVGAVVLLATIFIILLSISLCKRRKNRAGQKGKNTPSRLA

SEQ ID NO: 3161 - protein, 238 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKYSTSLELWYLWSSSLSSSWLYLYTSVERQEWGRAGRRTPH

SEQ ID NO: 3162 - protein, 294 aa, Mus musculus
MAILVRPRLLAALAPTFLGCLLLQVTAGAGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGETLIIVGAVVLLATIFIILLSISLCKRRKNRAGQKGKNTPSRLA

SEQ ID NO: 3163 - protein, 450 aa, synthetic construct
  REGION 1..450: note = Synthetic
MEVQLVESGGGLVQPGGSLRLSCAASGFNISSYWIHWVRQAPGKGLEWVGYIDPYSGSTYYADSVKGRFTISADTSKNTAYLQMNSLRAEDTAVYYCARSDYYYVDEYMDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3164 - protein, 449 aa, synthetic construct
  REGION 1..449: note = Synthetic
MEVQLVESGGGLVQPGGSLRLSCAASGFNISDYWIHWVRQAPGKGLEWVGFIDPYNGYTNYADSVKGRFTISADTSKNTAYLQMNSLRAEDTAVYYCARGYGSWPDYLDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3165 - protein, 449 aa, synthetic construct
  REGION 1..449: note = Synthetic
MEVQLVESGGGLVQPGGSLRLSCAASGFNISDYNIHWVRQAPGKGLEWVGYIDPSNGYTYYADSVKGRFTISADTSKNTAYLQMNSLRAEDTAVYYCARDDQSVSSPFDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3166-3167 - skipped (2 entries)

SEQ ID NO: 3168 - protein, 449 aa, synthetic construct
  REGION 1..449: note = Synthetic
MEVQLVESGGGLVQPGGSLRLSCAASGFTISGYIIHWVRQAPGKGLEWVGYIDPSNGYTYYADSVKGRFTISADTSKNTAYLQMNSLRAEDTAVYYCARDDQSLSSPFDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3169 - protein, 446 aa, synthetic construct
  REGION 1..446: note = Synthetic
MEVQLVESGGGLVQPGGSLRLSCAASGFSISSYDMSWVRQAPGKGLEWIGIIGSNGRTYYPDSVKGRFTISRDNSKNTVTLQMNSLRAEDTAVYYCARGLYDGTGNIWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3170 - protein, 449 aa, synthetic construct
  REGION 1..449: note = Synthetic
MEVQLVESGGGLVQPGGSLRLSCAASGFTISQYWIHWVRQAPGKGLEWVGYIDPYNGYTYYADSVKGRFTISADTSKNTAYLQMNSLRAEDTAVYYCARDYGSWRIRLDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3171 - protein, 449 aa, synthetic construct
  REGION 1..449: note = Synthetic
MEVQLVESGGGLVQPGGSLRLSCAASGFTISIYTIHWVRQAPGKGLEWVGYIDPSNGYTYYADSVKGRLTISADTSKNTAYLQMNSLRAEDTAVYYCARDPQSISSPFDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3172 - protein, 449 aa, synthetic construct
  REGION 1..449: note = Synthetic
MEVQLVESGGGLVQPGGSLRLSCAASGFTIHDYVIHWVRQAPGKGLEWVGYIDPSNGYTYYADSVKGRFTISADTSKNTAYLQMNSLRAEDTAVYYCARDDQSISSPLDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3173 - protein, 449 aa, synthetic construct
  REGION 1..449: note = Synthetic
MEVQLVESGGGLVQPGGSLRLSCAASGFTIAAYIIHWVRQAPGKGLEWVGYIDPSNGYTYYADSVKGRFTISADTSKNTAYLQMNSLRAEDTAVYYCARDDQSFSSPFDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3174 - protein, 449 aa, synthetic construct
  REGION 1..449: note = Synthetic
MEVQLVESGGGLVQPGGSLRLSCAASGFTISDYVIHWVRQAPGKGLEWVGYIDPSNGYTYYADSVKGRFTISADTSKNTAYLQMNSLRAEDTAVYYCARDDQSISSPLDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3175 - protein, 446 aa, synthetic construct
  REGION 1..446: note = Synthetic
MQVQVVESGGGLVKPGGSLRLSCAASGFSISSYDMSWIRQAPGKGLEWIGIIGSNGRTYYADSVKGRFTISRNTNENTVYLQMNSLRAEDTAVYYCARGLYDGTGNIWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3176 - protein, 449 aa, synthetic construct
  REGION 1..449: note = Synthetic
MQVQVQESGPGLVKPPGTLSLTCAVSGFSLSYYGVSWVRQPPGKGLEWIGTIDSSGRTYYNPSLKSRVTISNDNAPNTVSLKLSSVTAADTAVYYCARDFYGWNSGALDIWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3177 - protein, 448 aa, synthetic construct
  REGION 1..448: note = Synthetic
MEVQLVESGGGLVQPGGSLRLSCAASGFTISGYIIHWVRQAPGKGLEWVGYIDPSNGYTYYADSVKGRFTISADTSKNTAYLQMNSLRAEDTAVYYCARDDQSLSSPFDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3178 - protein, 449 aa, synthetic construct
  REGION 1..449: note = Synthetic
MEVQLVESGGGLVQPGGSLRLSCAASGFTISGYIIHWVRQAPGKGLEWVGYIDPSNGYTYYADSVKGRFTISADTSKNTAYLQMNSLRAEDTAVYYCARDDQSLSSPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 3179 - protein, 215 aa, synthetic construct
  REGION 1..215: note = Synthetic
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3180 - protein, 220 aa, synthetic construct
  REGION 1..220: note = Synthetic
MELVMTQSPPTLSLSPGERVTLSCRASQSVYGNNWLSWYQQKPGQAPRLLIYGASKLATSIPARFSGSGSGTQFTLTISSLQPEDFAVYYCQGTYYSGDWYFAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3181 - protein, 220 aa, synthetic construct
  REGION 1..220: note = Synthetic
MDLQMTQSPSSLSASVGDRVTITCRASQSVYGNNWLSWYQQKPGKAPKLLIYGASKLASGVPSRFSGSGSGTQFTLTISSLQPEDFATYYCQGTYYSGDWYFAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3182 - protein, 220 aa, synthetic construct
  REGION 1..220: note = Synthetic
MDIQMTQSPSSLSASVGDRVTITCQASQSVYGNNWLSWYQQKPGKAPKLLIYGASKLASGVPSRFSGSGSGTDFTLTISSLQPEDFATYYCQGTYYSGDWYFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3183 - protein, 219 aa, synthetic construct
  REGION 1..219: note = Synthetic
MDLQLTQSPSTLSASVGDRVTITCRASQSVYSNKYLSWYQQKPGKAPKLLIYKASTLASGVPSRFSGSGSGTEFTLTISSLQPDDFATYYCAAAYSDDSDTAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3184 - protein, 213 aa, synthetic construct
  REGION 1..213: note = Synthetic
MDIQMTQSPSSLSASVGDRVTITCRASQDVNTAVAWYQQKPGAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQSVTEQDSKDSTYSLSSTLTLSKADYEKHKVYACEVTHQGLSSPVTKSFNRGEC

SEQ ID NO: 3185 - protein, 214 aa, synthetic construct
  REGION 1..214: note = Synthetic
MDIQMTQSPSSLSASVGDRVTITCRASQDVNTAVAWYQQKPGAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3186 - protein, 449 aa, synthetic construct; identical to SEQ ID NO: 3170
  REGION 1..449: note = Synthetic

SEQ ID NO: 3187 - protein, 449 aa, synthetic construct; identical to SEQ ID NO: 3171
  REGION 1..449: note = Synthetic

SEQ ID NO: 3188 - protein, 449 aa, synthetic construct; identical to SEQ ID NO: 3172
  REGION 1..449: note = Synthetic

SEQ ID NO: 3189 - protein, 449 aa, synthetic construct; identical to SEQ ID NO: 3173
  REGION 1..449: note = Synthetic

SEQ ID NO: 3190 - protein, 449 aa, synthetic construct; identical to SEQ ID NO: 3174
  REGION 1..449: note = Synthetic

SEQ ID NO: 3191 - protein, 447 aa, synthetic construct
  REGION 1..447: note = Synthetic
MEVQLVESGGGLVQPGGSLRLSCAASGFTISGYIIHWVRQAPGKGLEWVGYIDPSNGYTYYADSVKGRFTISADTSKNTAYLQMNSLRAEDTAVYYCARDDQSLSSPFDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNKTTPPVLDSDGSFLYSKLTVDKSRWQQGNVFSCSVMHEALHNHYTQKSLSLSPGK